SEQUENCE LISTING (519 sequences)

SEQ ID NO: 1 - DNA, 59 bp, synthetic construct
  misc_feature 1..59: note = Description of Artificial Sequence: Synthetic primer
  source 1..59: mol_type = other DNA
gacgaattcgctagcaagaggagaagtcgacatgtccaattcactggccgtcgttttac

SEQ ID NO: 2 - DNA, 47 bp, synthetic construct
  misc_feature 1..47: note = Description of Artificial Sequence: Synthetic primer
  source 1..47: mol_type = other DNA
gaccctaggaagctttctagagtcgacctatgcggcatcagagcaga

SEQ ID NO: 3 - DNA, 18 bp, synthetic construct
  misc_feature 1..18: note = Description of Artificial Sequence: Synthetic primer
  source 1..18: mol_type = other DNA
atgtaccgcaagttccgc

SEQ ID NO: 4 - DNA, 18 bp, synthetic construct
  misc_feature 1..18: note = Description of Artificial Sequence: Synthetic primer
  source 1..18: mol_type = other DNA
caatttgccgatgcccag

SEQ ID NO: 5 - DNA, 20 bp, synthetic construct
  misc_feature 1..20: note = Description of Artificial Sequence: Synthetic primer
  source 1..20: mol_type = other DNA
gctgaccactgaagactttg

SEQ ID NO: 6 - DNA, 19 bp, synthetic construct
  misc_feature 1..19: note = Description of Artificial Sequence: Synthetic primer
  source 1..19: mol_type = other DNA
gatcagggcttcggtgtag

SEQ ID NO: 7 - DNA, 27 bp, synthetic construct
  misc_feature 1..27: note = Description of Artificial Sequence: Synthetic primer
  source 1..27: mol_type = other DNA
ttggtgcgggccaagcaggatctgctc

SEQ ID NO: 8 - DNA, 30 bp, synthetic construct
  misc_feature 1..30: note = Description of Artificial Sequence: Synthetic primer
  source 1..30: mol_type = other DNA
tcagccgaacgcctcgtcgaggatctcctg

SEQ ID NO: 9 - DNA, 32 bp, synthetic construct
  misc_feature 1..32: note = Description of Artificial Sequence: Synthetic primer
  source 1..32: mol_type = other DNA
tggccaacataagttcaccattcgggcaaaac

SEQ ID NO: 10 - DNA, 29 bp, synthetic construct
  misc_feature 1..29: note = Description of Artificial Sequence: Synthetic primer
  source 1..29: mol_type = other DNA
tctcttcaaccagccattcgttttgcccg

SEQ ID NO: 11 - DNA, 27 bp, Clostridium acetobutylicum
  source 1..27: mol_type = other DNA
attaaagttaagtggaggaatgttaac

SEQ ID NO: 12 - DNA, 22 bp, synthetic construct
  misc_feature 1..22: note = Description of Artificial Sequence: Synthetic primer
  source 1..22: mol_type = other DNA
acacgcggatccaacgtcccgg

SEQ ID NO: 13 - DNA, 23 bp, synthetic construct
  misc_feature 1..23: note = Description of Artificial Sequence: Synthetic primer
  source 1..23: mol_type = other DNA
agcggctccgctagccgcttatg

SEQ ID NO: 14 - DNA, 24 bp, synthetic construct
  misc_feature 1..24: note = Description of Artificial Sequence: Synthetic primer
  source 1..24: mol_type = other DNA
aagccgttgctgcagctcttgagc

SEQ ID NO: 15 - DNA, 23 bp, synthetic construct
  misc_feature 1..23: note = Description of Artificial Sequence: Synthetic primer
  source 1..23: mol_type = other DNA
atctccggcggtcggatccgtcg

SEQ ID NO: 16 - DNA, 20 bp, synthetic construct
  misc_feature 1..20: note = Description of Artificial Sequence: Synthetic primer
  source 1..20: mol_type = other DNA
aaagcggctagccacgccgc

SEQ ID NO: 17 - DNA, 20 bp, synthetic construct
  misc_feature 1..20: note = Description of Artificial Sequence: Synthetic primer
  source 1..20: mol_type = other DNA
attacacgaggtacccaacg

SEQ ID NO: 18 - DNA, 22 bp, synthetic construct
  misc_feature 1..22: note = Description of Artificial Sequence: Synthetic primer
  source 1..22: mol_type = other DNA
atgctggcgtacaaaggtgtcc

SEQ ID NO: 19 - DNA, 22 bp, synthetic construct
  misc_feature 1..22: note = Description of Artificial Sequence: Synthetic primer
  source 1..22: mol_type = other DNA
ggacacctttgtacgccagcat

SEQ ID NO: 20 - DNA, 25 bp, synthetic construct
  misc_feature 1..25: note = Description of Artificial Sequence: Synthetic primer
  source 1..25: mol_type = other DNA
atcgcctacactaaaccagaagtgg

SEQ ID NO: 21 - DNA, 25 bp, synthetic construct
  misc_feature 1..25: note = Description of Artificial Sequence: Synthetic primer
  source 1..25: mol_type = other DNA
ccacttctggtttagtgtaggcgat

SEQ ID NO: 22 - DNA, 20 bp, synthetic construct
  misc_feature 1..20: note = Description of Artificial Sequence: Synthetic primer
  source 1..20: mol_type = other DNA
aggcagttccataggatggc

SEQ ID NO: 23 - DNA, 55 bp, synthetic construct
  misc_feature 1..55: note = Description of Artificial Sequence: Synthetic primer
  source 1..55: mol_type = other DNA
tgacatgtaacacctaccttctgtgcctgtgccagtggttgctgtgatatagaag

SEQ ID NO: 24 - DNA, 48 bp, synthetic construct
  misc_feature 1..48: note = Description of Artificial Sequence: Synthetic primer
  source 1..48: mol_type = other DNA
ataataatacatatgaaccatgcgagttacgggcctataagccaggcg

SEQ ID NO: 25 - DNA, 42 bp, synthetic construct
  misc_feature 1..42: note = Description of Artificial Sequence: Synthetic primer
  source 1..42: mol_type = other DNA
agtttttcgatatctgcatcagacaccggcacattgaaacgg

SEQ ID NO: 26 - DNA, 60 bp, synthetic construct
  misc_feature 1..60: note = Description of Artificial Sequence: Synthetic primer
  source 1..60: mol_type = other DNA
ctggcacaggcacagaaggtaggtgttacatgtcagaacgtttacacaatgacgtggatc

SEQ ID NO: 27 - DNA, 49 bp, synthetic construct
  misc_feature 1..49: note = Description of Artificial Sequence: Synthetic primer
  source 1..49: mol_type = other DNA
agacaaatcggttgccgtttgttaagccaggcgagatatgatctatatc

SEQ ID NO: 28 - DNA, 54 bp, synthetic construct
  misc_feature 1..54: note = Description of Artificial Sequence: Synthetic primer
  source 1..54: mol_type = other DNA
gagttttgatttcagtactcatcatgtaacacctaccttcttgctgtgatatag

SEQ ID NO: 29 - DNA, 54 bp, synthetic construct
  misc_feature 1..54: note = Description of Artificial Sequence: Synthetic primer
  source 1..54: mol_type = other DNA
ctatatcacagcaagaaggtaggtgttacatgatgagtactgaaatcaaaactc

SEQ ID NO: 30 - DNA, 49 bp, synthetic construct
  misc_feature 1..49: note = Description of Artificial Sequence: Synthetic primer
  source 1..49: mol_type = other DNA
gatatagatcatatctcgcctggcttaacaaacggcaaccgatttgtct

SEQ ID NO: 31 - DNA, 70 bp, synthetic construct
  misc_feature 1..70: note = Description of Artificial Sequence: Synthetic oligonucleotide
  source 1..70: mol_type = other DNA
tattgtgcatacagatgaatttttatgcaaacagtcagccctgaagaagggtgtaggctggagctgcttc

SEQ ID NO: 32 - DNA, 70 bp, synthetic construct
  misc_feature 1..70: note = Description of Artificial Sequence: Synthetic oligonucleotide
  source 1..70: mol_type = other DNA
caaaaaaccggagtctgtgctccggttttttattatccgctaatcaattacatatgaatatcctccttag

SEQ ID NO: 33 - DNA, 51 bp, synthetic construct
  misc_feature 1..51: note = Description of Artificial Sequence: Synthetic primer
  source 1..51: mol_type = other DNA
ataataatagaattcgtttgctacctaaattgccaactaaatcgaaacagg

SEQ ID NO: 34 - DNA, 47 bp, synthetic construct
  misc_feature 1..47: note = Description of Artificial Sequence: Synthetic primer
  source 1..47: mol_type = other DNA
tattattatggtaccaatatcatgcagcaaacggtgcaacattgccg

SEQ ID NO: 35 - DNA, 47 bp, synthetic construct
  misc_feature 1..47: note = Description of Artificial Sequence: Synthetic primer
  source 1..47: mol_type = other DNA
tgatctggaagaattcatcggctttaccaccgtcaaaaaaaacggcg

SEQ ID NO: 36 - DNA, 48 bp, synthetic construct
  misc_feature 1..48: note = Description of Artificial Sequence: Synthetic primer
  source 1..48: mol_type = other DNA
ataaaaccctgcagcggaaacgaagttttatccatttttggttacctg

SEQ ID NO: 37 - DNA, 35 bp, synthetic construct
  misc_feature 1..35: note = Description of Artificial Sequence: Synthetic primer
  source 1..35: mol_type = other DNA
ggaagagaggctggtacccagaagccacagcagga

SEQ ID NO: 38 - DNA, 38 bp, synthetic construct
  misc_feature 1..38: note = Description of Artificial Sequence: Synthetic primer
  source 1..38: mol_type = other DNA
gtaatcactgcgtaagcgccatgccccggcgttaattc

SEQ ID NO: 39 - DNA, 25 bp, synthetic construct
  misc_feature 1..25: note = Description of Artificial Sequence: Synthetic primer
  source 1..25: mol_type = other DNA
attgccgcgttcctcctgctgtcga

SEQ ID NO: 40 - DNA, 24 bp, synthetic construct
  misc_feature 1..24: note = Description of Artificial Sequence: Synthetic primer
  source 1..24: mol_type = other DNA
cgacagcaggaggaacgcggcaat

SEQ ID NO: 41 - DNA, 75 bp, synthetic construct
  misc_feature 1..75: note = Description of Artificial Sequence: Synthetic primer
  source 1..75: mol_type = other DNA
gtttgcacgctatagctgaggttgttgtcttccagcaacgtaccgtatacaataggcgtatcacgaggccctttc

SEQ ID NO: 42 - DNA, 70 bp, synthetic construct
  misc_feature 1..70: note = Description of Artificial Sequence: Synthetic primer
  source 1..70: mol_type = other DNA
gctacagcatgtcacacgatctcaacggtcggatgaccaatctggctggtatgggaattagccatggtcc

SEQ ID NO: 43 - DNA, 73 bp, synthetic construct
  misc_feature 1..73: note = Description of Artificial Sequence: Synthetic oligonucleotide
  source 1..73: mol_type = other DNA
tgtgagtgaaagtcacctgccttaatatctcaaaactcatcttcgggtgacgaaatatggcgtgactcgatac

SEQ ID NO: 44 - DNA, 70 bp, synthetic construct
  misc_feature 1..70: note = Description of Artificial Sequence: Synthetic oligonucleotide
  source 1..70: mol_type = other DNA
tctgtatcaggctgaaaatcttctctcatccgccaaaacagcttcggcgttaagatgcgcgctcaaggac

SEQ ID NO: 45 - protein, 20 aa, Euglena gracilis
MTYKAPVKDVKFLLDKVFKV

SEQ ID NO: 46 - DNA, 2036 bp, Escherichia coli
  source 1..2036: mol_type = other DNA
atgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataatgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataa

SEQ ID NO: 47 - protein, 388 aa, Escherichia coli
MNLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGK

SEQ ID NO: 48 - protein, 289 aa, Escherichia coli
MSILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

SEQ ID NO: 49 - DNA, 3696 bp, Mycobacterium bovis
  source 1..3696: mol_type = other DNA
atggccaacataagttcaccattcgggcaaaacgaatggctggttgaagagatgtaccgcaagttccgcgacgacccctcctcggtcgatcccagctggcacgagttcctggttgactacagccccgaacccacctcccaaccagctgccgaaccaacccgggttacctcgccactcgttgccgagcgggccgctgcggccgccccgcaggcaccccccaagccggccgacaccgcggccgcgggcaacggcgtggtcgccgcactggccgccaaaactgccgttcccccgccagccgaaggtgacgaggtagcggtgctgcgcggcgccgccgcggccgtcgtcaagaacatgtccgcgtcgttggaggtgccgacggcgaccagcgtccgggcggtcccggccaagctactgatcgacaaccggatcgtcatcaacaaccagttgaagcggacccgcggcggcaagatctcgttcacgcatttgctgggctacgccctggtgcaggcggtgaagaaattcccgaacatgaaccggcactacaccgaagtcgacggcaagcccaccgcggtcacgccggcgcacaccaatctcggcctggcgatcgacctgcaaggcaaggacgggaagcgttccctggtggtggccggcatcaagcggtgcgagaccatgcgattcgcgcagttcgtcacggcctacgaagacatcgtacgccgggcccgcgacggcaagctgaccactgaagactttgccggcgtgacgatttcgctgaccaatcccggaaccatcggcaccgtgcattcggtgccgcggctgatgcccggccagggcgccatcatcggcgtgggcgccatggaataccccgccgagtttcaaggcgccagcgaggaacgcatcgccgagctgggcatcggcaaattgatcactttgacctccacctacgaccaccgcatcatccagggcgcggaatcgggcgacttcctgcgcaccatccacgagttgctgctctcggatggcttctgggacgaggtcttccgcgaactgagcatcccatatctgccggtgcgctggagcaccgacaaccccgactcgatcgtcgacaagaacgctcgcgtcatgaacttgatcgcggcctaccgcaaccgcggccatctgatggccgataccgacccgctgcggttggacaaagctcggttccgcagtcaccccgacctcgaagtgctgacccacggcctgacgctgtgggatctcgatcgggtgttcaaggtcgacggctttgccggtgcgcagtacaagaaactgcgcgacgtgctgggcttgctgcgcgatgcctactgccgccacatcggcgtggagtacgcccatatcctcgaccccgaacaaaaggagtggctcgaacaacgggtcgagaccaagcacgtcaaacccactgtggcccaacagaaatacatcctcagcaagctcaacgccgccgaggcctttgaaacgttcctacagaccaagtacgtcggccagaagcggttctcgctggaaggcgccgaaagcgtgatcccgatgatggacgcggcgatcgaccagtgcgctgagcacggcctcgacgaggtggtcatcgggatgccgcaccggggccggctcaacgtgctggccaacatcgtcggcaagccgtactcgcagatcttcaccgagttcgagggcaacctgaatccgtcgcaggcgcacggctccggtgacgtcaagtaccacctgggcgccaccgggctgtacctgcagatgttcggcgacaacgacattcaggtgtcgctgaccgccaacccgtcgcatctggaggccgtcgacccggtgctggagggattggtgcgggccaagcaggatctgctcgaccacggaagcatcgacagcgacggccaacgggcgttctcggtggtgccgctgatgttgcatggcgatgccgcgttcgccggtcagggtgtggtcgccgagacgctgaacctggcgaatctgccgggctaccgcgtcggcggcaccatccacatcatcgtcaacaaccagatcggcttcaccaccgcgcccgagtattccaggtccagcgagtactgcaccgacgtcgcaaagatgatcggggcaccgatctttcacgtcaacggcgacgacccggaggcgtgtgtctgggtggcgcggttggcggtggacttccgacaacggttcaagaaggacgtcgtcatcgacatgctgtgctaccgccgccgcgggcacaacgagggtgacgacccgtcgatgaccaacccctacatgtacgacgtcgtcgacaccaagcgcggggcccgcaaaagctacaccgaagccctgatcggacgtggcgacatctcgatgaaggaggccgaggacgcgctgcgcgactaccagggccagctggaacgggtgttcaacgaagtgcgcgagctggagaagcacggtgtgcagccgagcgagtcggtcgagtccgaccagatgattcccgcggggctggccactgcggtggacaagtcgctgctggcccggatcggcgatgcgttcctcgccttgccgaacggcttcaccgcgcacccgcgagtccaaccggtgctggagaagcgccgggagatggcctatgaaggcaagatcgactgggcctttggcgagctgctggcgctgggctcgctggtggccgaaggcaagctggtgcgcttgtcggggcaggacagccgccgcggcaccttctcccagcggcattcggttctcatcgaccgccacactggcgaggagttcacaccactgcagctgctggcgaccaactccgacggcagcccgaccggcggaaagttcctggtctacgactcgccactgtcggagtacgccgccgtcggcttcgagtacggctacactgtgggcaatccggacgccgtggtgctctgggaggcgcagttcggcgacttcgtcaacggcgcacagtcgatcatcgacgagttcatcagctccggtgaggccaagtggggccaattgtccaacgtcgtgctgctgttaccgcacgggcacgaggggcagggacccgaccacacttctgcccggatcgaacgcttcttgcagttgtgggcggaaggttcgatgaccatcgcgatgccgtcgactccgtcgaactacttccacctgctacgccggcatgccctggacggcatccaacgcccgctgatcgtgttcacgcccaagtcgatgttgcgtcacaaggccgccgtcagcgaaatcaaggacttcaccgagatcaagttccgctcagtgctggaggaacccacctatgaggacggcatcggagaccgcaacaaggtcagccggatcctgctgaccagtggcaagctgtattacgagctggccgcccgcaaggccaaggacaaccgcaatgacctcgcgatcgtgcggcttgaacagctcgccccgctgcccaggcgtcgactgcgtgaaacgctggaccgctacgagaacgtcaaggagttcttctgggtccaagaggaaccggccaaccagggtgcgtggccgcgattcgggctcgaactacccgagctgctgcctgacaagttggccgggatcaagcgaatctcgcgccgggcgatgtcagccccgtcgtcaggctcgtcgaaggtgcacgccgtcgaacagcaggagatcctcgacgaggcgttcggctaa

SEQ ID NO: 50 - protein, 1231 aa, Mycobacterium bovis
MANISSPFGQNEWLVEEMYRKFRDDPSSVDPSWHEFLVDYSPEPTSQPAAEPTRVTSPLVAERAAAAAPQAPPKPADTAAAGNGVVAALAAKTAVPPPAEGDEVAVLRGAAAAVVKNMSASLEVPTATSVRAVPAKLLIDNRIVINNQLKRTRGGKISFTHLLGYALVQAVKKFPNMNRHYTEVDGKPTAVTPAHTNLGLAIDLQGKDGKRSLVVAGIKRCETMRFAQFVTAYEDIVRRARDGKLTTEDFAGVTISLTNPGTIGTVHSVPRLMPGQGAIIGVGAMEYPAEFQGASEERIAELGIGKLITLTSTYDHRIIQGAESGDFLRTIHELLLSDGFWDEVFRELSIPYLPVRWSTDNPDSIVDKNARVMNLIAAYRNRGHLMADTDPLRLDKARFRSHPDLEVLTHGLTLWDLDRVFKVDGFAGAQYKKLRDVLGLLRDAYCRHIGVEYAHILDPEQKEWLEQRVETKHVKPTVAQQKYILSKLNAAEAFETFLQTKYVGQKRFSLEGAESVIPMMDAAIDQCAEHGLDEVVIGMPHRGRLNVLANIVGKPYSQIFTEFEGNLNPSQAHGSGDVKYHLGATGLYLQMFGDNDIQVSLTANPSHLEAVDPVLEGLVRAKQDLLDHGSIDSDGQRAFSVVPLMLHGDAAFAGQGVVAETLNLANLPGYRVGGTIHIIVNNQIGFTTAPEYSRSSEYCTDVAKMIGAPIFHVNGDDPEACVWVARLAVDFRQRFKKDVVIDMLCYRRRGHNEGDDPSMTNPYMYDVVDTKRGARKSYTEALIGRGDISMKEAEDALRDYQGQLERVFNEVRELEKHGVQPSESVESDQMIPAGLATAVDKSLLARIGDAFLALPNGFTAHPRVQPVLEKRREMAYEGKIDWAFGELLALGSLVAEGKLVRLSGQDSRRGTFSQRHSVLIDRHTGEEFTPLQLLATNSDGSPTGGKFLVYDSPLSEYAAVGFEYGYTVGNPDAVVLWEAQFGDFVNGAQSIIDEFISSGEAKWGQLSNVVLLLPHGHEGQGPDHTSARIERFLQLWAEGSMTIAMPSTPSNYFHLLRRHALDGIQRPLIVFTPKSMLRHKAAVSEIKDFTEIKFRSVLEEPTYEDGIGDRNKVSRILLTSGKLYYELAARKAKDNRNDLAIVRLEQLAPLPRRRLRETLDRYENVKEFFWVQEEPANQGAWPRFGLELPELLPDKLAGIKRISRRAMSAPSSGSSKVHAVEQQEILDEAFG

SEQ ID NO: 51 - DNA, 1356 bp, Porphyromonas gingivalis
  source 1..1356: mol_type = other DNA
atggaaatcaaagaaatggtgagccttgcacgcaaggctcagaaggagtatcaagctacccataaccaagaagcagttgacaacatttgccgagctgcagcaaaagttatttatgaaaatgcagctattctggctcgcgaagcagtagacgaaaccggcatgggcgtttacgaacacaaagtggccaagaatcaaggcaaatccaaaggtgtttggtacaacctccacaataaaaaatcgattggtatcctcaatatagacgagcgtaccggtatgatcgagattgcaaagcctatcggagttgtaggagccgtaacgccgacgaccaacccgatcgttactccgatgagcaatatcatctttgctcttaagacctgcaatgccatcattattgccccccaccccagatccaaaaaatgctctgcacacgcagttcgtctgatcaaagaagctatcgctccgttcaacgtaccggaaggtatggttcagatcatcgaagaacccagcatcgagaagacgcaggaactcatgggcgccgtagacgtagtagttgctacgggtggtatgggcatggtgaagtctgcatattcttcaggaaagccttctttcggtgttggagccggtaacgttcaggtgatcgtggatagcaacatcgatttcgaagctgctgcagaaaaaatcatcaccggtcgtgctttcgacaacggtatcatctgctcaggcgaacagagcatcatctacaacgaggctgacaaggaagcagttttcacagcattccgcaaccacggtgcatatttctgtgacgaagccgaaggagatcgggctcgtgcagctatcttcgaaaatggagccatcgcgaaagatgtagtaggtcagagcgttgccttcattgccaagaaagcaaacatcaatatccccgagggtacccgtattctcgttgttgaagctcgcggcgtaggagcagaagacgttatctgtaaggaaaagatgtgtcccgtaatgtgcgccctcagctacaagcacttcgaagaaggtgtagaaatcgcacgtacgaacctcgccaacgaaggtaacggccacacctgtgctatccactccaacaatcaggcacacatcatcctcgcaggatcagagctgacggtatctcgtatcgtagtgaatgctccgagtgccactacagcaggcggtcacatccaaaacggtcttgccgtaaccaatacgctcggatgcggatcatggggtaataactctatctccgagaacttcacttacaagcacctcctcaacatttcacgcatcgcaccgttgaattcaagcattcacatccccgatgacaaagaaatctgggaactctaa

SEQ ID NO: 52 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 53 - DNA, 1116 bp, Porphyromonas gingivalis
  source 1..1116: mol_type = other DNA
atgcaacttttcaaactcaagagtgtaacacatcactttgacacttttgcagaatttgccaaggaattctgtcttggagaacgcgacttggtaattaccaacgagttcatctatgaaccgtatatgaaggcatgccagctcccctgccattttgttatgcaggagaaatatgggcaaggcgagccttctgacgaaatgatgaataacatcttggcagacatccgtaatatccagttcgaccgcgtaatcggtatcggaggaggtacggttattgacatctctaaacttttcgttctgaaaggattaaatgatgtactcgatgcattcgaccgcaaaatacctcttatcaaagagaaagaactgatcattgtgcccacaacatgcggaacgggtagcgaggtgacgaacatttctatcgcagaaatcaaaagccgtcacaccaaaatgggattggctgacgatgccattgttgcagaccatgccatcatcatacctgaacttctgaagagcttgcctttccacttctacgcatgcagtgcaatcgatgctcttatccatgccatcgagtcatacgtatctcctaaagccagtccatattctcgtctgttcagtgaggcggcttgggacattatcctggaagtattcaagaaaatcgccgaacacggccctgaataccgcttcgaaaagctgggagaaatgatcatggccagcaactatgccggtatagccttcggaaatgcaggagtaggagccgtccacgcactatcctacccgttgggaggcaactatcacgtgccgcatggagaagcaaactatcagttcttcacagaggtattcaaagtataccaaaagaagaatcctttcggctatatagtcgaactcaactggaagctctccaagatactgaactgccagcccgaatacgtatatccgaagctggatgaacttctcggatgccttcttaccaagaaacctttgcacgaatacggcatgaaggacgaagaggtaagaggctttgcggaatcagtgcttaagacacagcaaagattgctcgccaacaactacgtagagcttactgtagatgagatcgaaggtatctacagaagactctactaa

SEQ ID NO: 54 - protein, 371 aa, Porphyromonas gingivalis
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 55 - DNA, 1296 bp, Porphyromonas gingivalis
  source 1..1296: mol_type = other DNA
atgaaagacgtattagcggaatatgcctcccgaattgtttcggccgaagaagccgtaaaacatatcaaaaatggagaacgggtagctttgtcacatgctgccggagttcctcagagttgtgttgatgcactggtacaacaggccgaccttttccagaatgtcgaaatttatcacatgctttgtctcggcgaaggaaaatatatggcacctgaaatggcccctcacttccgacacataaccaattttgtaggtggtaattctcgtaaagcagttgaggaaaatagagccgacttcattccggtattcttttatgaagtgccatcaatgattcgcaaagacatccttcacatagatgtcgccatcgttcagctttcaatgcctgatgagaatggttactgtagttttggagtatcttgcgattatagcaaaccggcagcagaaagcgctcatttagttataggggaaatcaaccgtcaaatgccatatgtacatggcgacaacttgattcacatatcgaagttggattacatcgtgatggcagactaccctatctattctcttgcaaagcccaaaatcggagaagtagaagaagctatcgggcgtaattgtgccgagcttattgaagatggtgccacactccaactcggtatcggcgcgattcctgatgcagccctgttattcctcaaggacaaaaaagatctggggatccataccgagatgttctccgatggtgttgtcgaattagttcgcagtggagtaattacaggaaagaaaaagacacttcaccccggaaagatggtcgcaaccttcttaatgggaagcgaagacgtatatcatttcatcgacaaaaatcccgatgtagaactttatccggtagattacgtcaatgatccgcgagtaatcgctcaaaatgataatatggtcagcatcaatagctgtatcgaaatcgatcttatgggacaagtcgtgtccgaatgtataggaagcaagcaattcagcggaaccggcggtcaagtagattatgttcgtggagcagcatggtctaaaaacggcaaaagcatcatggcaattccctcaacagccaaaaacggtactgcatctcgaattgtacctataattgcagagggagctgctgtaacaaccctccgcaacgaagtcgattacgttgtaaccgaatacggtatagcacaactcaaaggaaagagtttgcgccagcgagcagaagctcttattgccatagcccacccggatttcagagaggaactaacgaaacatctccgcaaacgtttcggataa

SEQ ID NO: 56 - protein, 431 aa, Porphyromonas gingivalis
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEIDLMGQVVSECIGSKQFSGTGGQVDYVRGAAWSKNGKSIMAIPSTAKNGTASRIVPIIAEGAAVTTLRNEVDYVVTEYGIAQLKGKSLRQRAEALIAIAHPDFREELTKHLRKRFG

SEQ ID NO: 57 - DNA, 906 bp, Clostridium acetobutylicum
  source 1..906: mol_type = other DNA
atgattaagagttttaatgaaattatcatgaaggtaaagagcaaagaaatgaaaaaagttgctgttgctgtagcacaagacgagccagtacttgaagcagtaagagatgctaagaaaaatggtattgcagatgctattcttgttggagaccatgacgaaatcgtgtcaatcgcgcttaaaataggaatggatgtaaatgattttgaaatagtaaacgagcctaacgttaagaaagctgctttaaaggcagtagagcttgtatcaactggaaaagctgatatggtaatgaagggacttgtaaatacagcaactttcttaagatctgtattaaacaaagaagttggacttagaacaggaaaaactatgtctcacgttgcagtatttgaaactgagaaatttgatagactattatttttaacagatgttgctttcaatacttatcctgaattaaaggaaaaaattgatatagtaaacaattcagttaaggttgcacatgcaataggaattgaaaatccaaaggttgctccaatttgtgcagttgaggttataaaccctaaaatgccatcaacacttgatgcagcaatgctttcaaaaatgagtgacagaggacaaattaaaggttgtgtagttgacggacctttagcacttgatatagctttatcagaagaagcagcacatcataagggagtaacaggagaagttgctggaaaagctgatatcttcttaatgccaaacatagaaacaggaaatgtaatgtataagactttaacatatacaactgattcaaaaaatggaggaatcttagttggaacttctgcaccagttgttttaacttcaagagctgacagccatgaaacaaaaatgaactctatagcacttgcagctttagttgcaggcaataaataa

SEQ ID NO: 58 - protein, 301 aa, Clostridium acetobutylicum
MIKSFNEIIMKVKSKEMKKVAVAVAQDEPVLEAVRDAKKNGIADAILVGDHDEIVSIALKIGMDVNDFEIVNEPNVKKAALKAVELVSTGKADMVMKGLVNTATFLRSVLNKEVGLRTGKTMSHVAVFETEKFDRLLFLTDVAFNTYPELKEKIDIVNNSVKVAHAIGIENPKVAPICAVEVINPKMPSTLDAAMLSKMSDRGQIKGCVVDGPLALDIALSEEAAHHKGVTGEVAGKADIFLMPNIETGNVMYKTLTYTTDSKNGGILVGTSAPVVLTSRADSHETKMNSIALAALVAGNK

SEQ ID NO: 59 - DNA, 1068 bp, Clostridium acetobutylicum
  source 1..1068: mol_type = other DNA
atgtatagattactaataatcaatcctggctcgacctcaactaaaattggtatttatgacgatgaaaaagagatatttgagaagactttaagacattcagctgaagagatagaaaaatataacactatatttgatcaatttcaattcagaaagaatgtaattttagatgcgttaaaagaagcaaacatagaagtaagttctttaaatgctgtagttggaagaggcggactcttaaagccaatagtaagtggaacttatgcagtaaatcaaaaaatgcttgaagaccttaaagtaggagttcaaggtcagcatgcgtcaaatcttggtggaattattgcaaatgaaatagcaaaagaaataaatgttccagcatacatagttgatccagttgttgtggatgagcttgatgaagtttcaagaatatcaggaatggctgacattccaagaaaaagtatattccatgcattaaatcaaaaagcagttgctagaagatatgcaaaagaagttggaaaaaaatacgaagatcttaatttaatcgtagtccacatgggtggaggtacttcagtaggtactcataaagatggtagagtaatagaagttaataatacacttgatggagaaggtccattctcaccagaaagaagtggtggagttccaataggagatcttgtaagattgtgcttcagcaacaaatatacttatgaagaagtaatgaaaaagataaacggcaaaggcggagttgttagttacttaaatactatcgattttaaggctgtagttgataaagctcttgaaggagataagaaatgtgcacttatatatgaagctttcacattccaggtagcaaaagagataggaaaatgttcaaccgttttaaaaggaaatgtagatgcaataatcttaacaggcggaattgcgtacaacgagcatgtatgtaatgccatagaggatagagtaaaattcatagcacctgtagttagatatggtggagaagatgaacttcttgcacttgcagaaggtggacttagagttttaagaggagaagaaaaagctaaggaatacaaataa

SEQ ID NO: 60 - protein, 355 aa, Clostridium acetobutylicum
MYRLLIINPGSTSTKIGIYDDEKEIFEKTLRHSAEEIEKYNTIFDQFQFRKNVILDALKEANIEVSSLNAVVGRGGLLKPIVSGTYAVNQKMLEDLKVGVQGQHASNLGGIIANEIAKEINVPAYIVDPVVVDELDEVSRISGMADIPRKSIFHALNQKAVARRYAKEVGKKYEDLNLIVVHMGGGTSVGTHKDGRVIEVNNTLDGEGPFSPERSGGVPIGDLVRLCFSNKYTYEEVMKKINGKGGVVSYLNTIDFKAVVDKALEGDKKCALIYEAFTFQVAKEIGKCSTVLKGNVDAIILTGGIAYNEHVCNAIEDRVKFIAPVVRYGGEDELLALAEGGLRVLRGEEKAKEYK

SEQ ID NO: 61 - DNA, 906 bp, synthetic construct
  misc_feature 1..906: note = Description of Artificial Sequence: Synthetic polynucleotide
  source 1..906: mol_type = other DNA
atgattaagagttttaatgaaattatcatgaaggtaaagagcaaagaaatgaaaaaagttgctgttgctgtagcacaagacgagccagtacttgaagcagtacgcgatgctaagaaaaatggtattgcagatgctattcttgttggcgaccatgacgaaatcgtgtcaatcgcgcttaaaataggcatggatgtaaatgattttgaaatagtaaacgagcctaacgttaagaaagctgctttaaaggcagtagagctggtatcaactggaaaagctgatatggtaatgaagggacttgtaaatacagcaactttcttacgctctgtattaaacaaagaagttggactgagaacaggaaaaactatgtctcacgttgcagtatttgaaactgagaaatttgatcgtctgttatttttaacagatgttgctttcaatacttatcctgaattaaaggaaaaaattgatatcgtaaacaattcagttaaggttgcacatgcaataggtattgaaaatccaaaggttgctccaatttgtgcagttgaggttataaaccctaaaatgccatcaacacttgatgcagcaatgctttcaaaaatgagtgacagaggacaaattaaaggttgtgtagttgacggaccgttagcacttgatatcgctttatcagaagaagcagcacatcataagggcgtaacaggagaagttgctggaaaagctgatatcttcttaatgccaaacattgaaacaggaaatgtaatgtataagactttaacatatacaactgatagcaaaaatggcggaatcttagttggaacttctgcaccagttgttttaacttcacgcgctgacagccatgaaacaaaaatgaactctattgcacttgcagctttagttgcaggcaataaataa

SEQ ID NO: 62 - DNA, 906 bp, synthetic construct
  misc_feature 1..906: note = Description of Artificial Sequence: Synthetic polynucleotide
  source 1..906: mol_type = other DNA
atgattaagagttttaatgaaattatcatgaaggtaaagagcaaagaaatgaaaaaagttgctgttgctgtagcacaagacgagccagtacttgaagcagtacgcgatgctaagaaaaatggtattgccgatgctattctggttggcgaccatgacgaaatcgtgtctatcgcgctgaaaataggcatggatgtaaatgattttgaaattgttaacgagcctaacgttaagaaagctgcgttaaaggcagtagagctggtatcaactggaaaagctgatatggtaatgaagggactggtaaataccgcaactttcttacgctctgtattaaacaaagaagttggtctgcgtacaggaaaaaccatgtctcacgttgcagtatttgaaactgagaaatttgatcgtctgttatttttaacagatgttgctttcaatacttatcctgaattaaaggaaaaaattgatatcgttaacaatagcgttaaggttgcacatgccattggtattgaaaatccaaaggttgctccaatttgtgcagttgaggttattaacccgaaaatgccatcaacacttgatgcagcaatgctttcaaaaatgagtgaccgcggacaaattaaaggttgtgtagttgacggaccgctggcacttgatatcgctttatcagaagaagcagcacatcataaaggcgtaacaggagaagttgctggaaaagctgatatcttcttaatgccaaacattgaaacaggaaatgtaatgtataagacgttaacctataccactgatagcaaaaatggcggcatcctggttggaacttctgcaccagttgttttaacttcacgcgctgacagccatgaaacaaaaatgaactctattgcactggcagcgctggttgcaggcaataaataa

SEQ ID NO: 63 - DNA, 906 bp, synthetic construct
  misc_feature 1..906: note = Description of Artificial Sequence: Synthetic polynucleotide
  source 1..906: mol_type = other DNA
atgattaagagttttaatgaaattatcatgaaggtaaagagcaaagaaatgaaaaaagttgctgttgctgttgcacaagacgagccggtactggaagcggtacgcgatgctaagaaaaatggtattgccgatgctattctggttggcgaccatgacgaaatcgtctctatcgcgctgaaaattggcatggatgttaatgattttgaaattgttaacgagcctaacgttaagaaagctgcgctgaaggcggtagagctggtttccaccggaaaagctgatatggtaatgaaagggctggtgaataccgcaactttcttacgcagcgtactgaacaaagaagttggtctgcgtaccggaaaaaccatgagtcacgttgcggtatttgaaactgagaaatttgatcgtctgctgtttctgaccgatgttgctttcaatacttatcctgaattaaaagaaaaaattgatatcgttaacaatagcgttaaggttgcgcatgccattggtattgaaaatccaaaggttgctccaatttgtgcagttgaggttattaacccgaaaatgccatcaacacttgatgccgcaatgcttagcaaaatgagtgaccgcggacaaattaaaggttgtgtggttgacggcccgctggcactggatatcgcgttaagcgaagaagcggcacatcataaaggcgtaaccggcgaagttgctggaaaagctgatatcttcctgatgccaaacattgaaacaggcaatgtaatgtataaaacgttaacctataccactgatagcaaaaatggcggcatcctggttggaacttctgcaccagttgttttaacctcacgcgctgacagccatgaaaccaaaatgaacagcattgcactggcagcgctggttgcaggcaataaataa

SEQ ID NO: 64 - DNA, 906 bp, synthetic construct
  misc_feature 1..906: note = Description of Artificial Sequence: Synthetic polynucleotide
  source 1..906: mol_type = other DNA
atgattaaaagttttaacgaaattatcatgaaagtgaaaagcaaagagatgaaaaaagtggcggttgcggttgcgcaggatgaaccggtgctggaagcggtgcgcgatgccaaaaaaaacggtattgccgatgccattctggtgggcgatcacgatgaaattgtctctattgcgctgaaaattggcatggatgttaacgattttgaaattgttaatgaaccgaacgtgaaaaaagcggcgctgaaagcggttgaactggtttccaccggtaaagccgatatggtgatgaaagggctggtgaataccgcaaccttcctgcgcagcgtgctgaataaagaagtgggtctgcgtaccggtaaaaccatgagtcatgttgcggtgtttgaaaccgaaaaatttgaccgtctgctgtttctgaccgatgttgcgtttaatacctatccggaactgaaagagaaaattgatatcgttaataacagcgtgaaagtggcgcatgccattggtattgaaaacccgaaagtggcgccgatttgcgcggttgaagtgattaacccgaaaatgccgtcaacgctggatgccgcgatgctcagcaaaatgagcgatcgcggtcaaatcaaaggctgtgtggttgatggcccgctggcgctggatatcgcgcttagcgaagaagcggcgcatcataaaggcgtgaccggcgaagtggccggtaaagccgatattttcctgatgccgaatattgaaaccggcaacgtgatgtataaaacgctgacctataccaccgacagcaaaaacggcggcattctggtgggtaccagcgcgccggtggtgctgacctcgcgcgccgacagccatgaaaccaaaatgaacagcattgcgctggcggcgctggtggccggtaataaataa

SEQ ID NO: 65 - DNA, 1068 bp, synthetic construct
  misc_feature 1..1068: note = Description of Artificial Sequence: Synthetic polynucleotide
  source 1..1068: mol_type = other DNA
atgtatcgtttactgattatcaatcctggctcgacctcaactaaaattggtatttatgacgatgaaaaagagatatttgagaagactttacgtcattcagctgaagagatagaaaaatataacactatatttgatcaatttcagttcagaaagaatgtaattctcgatgcgttaaaagaagcaaacattgaagtaagttctttaaatgctgtagttggacgcggcggactgttaaagccaatagtaagtggaacttatgcagtaaatcaaaaaatgcttgaagaccttaaagtaggcgttcaaggtcagcatgcgtcaaatcttggtggaattattgcaaatgaaatagcaaaagaaataaatgttccagcatacatcgttgatccagttgttgtggatgagcttgatgaagtttcacgtatatcaggaatggctgacattccacgtaaaagtatattccatgcattaaatcaaaaagcagttgctagacgctatgcaaaagaagttggaaaaaaatacgaagatcttaatttaatcgtggtccacatgggtggcggtacttcagtaggtactcataaagatggtagagtaattgaagttaataatacacttgatggagaaggtccattctcaccagaaagaagtggtggcgttccaataggcgatcttgtacgtttgtgcttcagcaacaaatatacttatgaagaagtaatgaaaaagataaacggcaaaggcggcgttgttagttacttaaatactatcgattttaaggctgtagttgataaagctcttgaaggcgataagaaatgtgcacttatatatgaagctttcacattccaggtagcaaaagagataggaaaatgttcaaccgttttaaaaggaaatgtagatgcaataatcttaacaggcggaattgcgtacaacgagcatgtatgtaatgccatagaggatagagtaaaattcattgcacctgtagttcgttatggtggagaagatgaacttcttgcacttgcagaaggtggactgcgcgttttacgcggagaagaaaaagctaaggaatacaaataa

SEQ ID NO: 66 - DNA, 1068 bp, synthetic construct
  misc_feature 1..1068: note = Description of Artificial Sequence: Synthetic polynucleotide
  source 1..1068: mol_type = other DNA
atgtatcgtttactgattatcaatcctggctcgacctcaactaaaattggtatttatgacgatgaaaaagagatatttgagaagacgttacgtcattcagctgaagagattgaaaaatataacactatatttgatcaatttcagttccgcaagaatgtgattctcgatgcgttaaaagaagcaaacattgaagtcagttctttaaatgctgtagttggacgcggcggactgttaaagccaattgtcagtggaacttatgcagtaaatcaaaaaatgcttgaagaccttaaagtgggcgttcaaggtcagcatgccagcaatcttggtggcattattgccaatgaaatcgcaaaagaaatcaatgttccagcatacatcgttgatccggttgttgtggatgagcttgatgaagttagccgtataagcggaatggctgacattccacgtaaaagtatattccatgcattaaatcaaaaagcagttgctcgtcgctatgcaaaagaagttggtaaaaaatacgaagatcttaatttaatcgtggtccacatgggtggcggtacttcagtaggtactcataaagatggtcgcgtgattgaagttaataatacacttgatggcgaaggtccattctcaccagaacgtagtggtggcgttccaattggcgatctggtacgtttgtgcttcagcaacaaatatacttatgaagaagtgatgaaaaagataaacggcaaaggcggcgttgttagttacctgaatactatcgattttaaggctgtagttgataaagcgcttgaaggcgataagaaatgtgcactgatttatgaagctttcaccttccaggtagcaaaagagattggtaaatgttcaaccgttttaaaaggaaatgttgatgccattatcttaacaggcggcattgcttacaacgagcatgtatgtaatgccattgaggatcgcgtaaaattcattgcacctgtagttcgttatggtggcgaagatgaactgctggcactggcagaaggtggactgcgcgttttacgcggcgaagaaaaagcgaaggaatacaaataa

SEQ ID NO: 67 - DNA, 1068 bp, synthetic construct
  misc_feature 1..1068: note = Description of Artificial Sequence: Synthetic polynucleotide
  source 1..1068: mol_type = other DNA
atgtatcgtctgctgattatcaatcctggctcgacctcaactaaaattggtatttatgacgatgaaaaagagatatttgagaaaacgttacgtcatagcgctgaagagattgaaaaatataacactatttttgatcaatttcagttccgcaagaatgtgattctcgatgcgctgaaagaagcaaacattgaagtcagttcgctgaatgcggtagttggtcgcggcggtctgctgaagccaattgtcagcggcacttatgcggtaaatcaaaaaatgctggaagacctgaaagtgggcgttcaggggcagcatgccagcaatcttggtggcattattgccaatgaaatcgccaaagaaatcaatgttccggcatacatcgttgatccggttgttgtggatgagctggatgaagttagccgtatcagcggaatggctgacattccacgtaaaagtattttccatgcactgaatcaaaaagcggttgcgcgtcgctatgcaaaagaagttggtaaaaaatacgaagatcttaatctgatcgtggtgcatatgggtggcggtactagcgtcggtactcataaagatggtcgcgtgattgaagttaataatacacttgatggcgaaggtccattctcaccagaacgtagcggtggcgttccaattggcgatctggtacgtttgtgcttcagcaacaaatatacctatgaagaagtgatgaaaaagataaacggcaaaggcggcgttgttagttacctgaatactatcgattttaaggcggtagttgataaagcgctggaaggcgataagaaatgtgcactgatttatgaagcgttcaccttccaggtggcaaaagagattggtaaatgttcaaccgttctgaaaggcaatgttgatgccattatcctgaccggcggcattgcttacaacgagcatgtttgtaatgccattgaggatcgcgtaaaattcattgcacctgtggttcgttatggtggcgaagatgaactgctggcactggcagaaggtggtctgcgcgttttacgcggcgaagaaaaagcgaaagaatacaaataa

SEQ ID NO: 68 - DNA, 1068 bp, synthetic construct
  misc_feature 1..1068: note = Description of Artificial Sequence: Synthetic polynucleotide
  source 1..1068: mol_type = other DNA
atgtatcgtctgctgattatcaacccgggcagcacctcaaccaaaattggtatttacgacgatgaaaaagagatttttgaaaaaacgctgcgtcacagcgcagaagagattgaaaaatacaacaccattttcgatcagttccagttccgcaaaaacgtgattctcgatgcgctgaaagaagccaatattgaagtctcctcgctgaatgcggtggtcggtcgcggcggtctgctgaaaccgattgtcagcggcacttatgcggttaatcagaaaatgctggaagatctgaaagtgggcgtgcaggggcagcatgccagcaatctcggcggcattatcgccaatgaaatcgccaaagagatcaacgtgccggcttatatcgtcgatccggtggtggttgatgaactggatgaagtcagccgtatcagcggcatggcggatattccgcgtaaaagcattttccatgcgctgaatcagaaagcggttgcgcgtcgctatgccaaagaagtgggtaaaaaatatgaagatctcaatctgattgtggtgcatatgggcggcggcaccagcgtcggtacgcataaagatggtcgcgtgattgaagtgaataacacgctggatggcgaagggccgttctcgccggaacgtagcggcggcgtgccgattggcgatctggtgcgtctgtgtttcagcaataaatacacctacgaagaagtgatgaaaaaaatcaacggcaaaggcggcgtggttagctatctgaataccatcgattttaaagcggtggttgataaagcgctggaaggcgataaaaaatgcgcgctgatttatgaagcgtttaccttccaggtggcgaaagagattggtaaatgttcaaccgtgctgaaaggcaacgttgatgccattattctgaccggcggcattgcttataacgaacatgtttgtaatgccattgaagatcgcgtgaaatttattgcgccggtggtgcgttacggcggcgaagatgaactgctggcgctggcggaaggcggtctgcgcgtgctgcgcggcgaagaaaaagcgaaagagtacaaataa

SEQ ID NO: 69 - DNA, 1407 bp, Clostridium beijerinckii
  source 1..1407: mol_type = other DNA
atgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaa

SEQ ID NO: 70 - protein, 468 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 71 - DNA, 1407 bp, synthetic construct
  misc_feature 1..1407: note = Description of Artificial Sequence: Synthetic polynucleotide
  source 1..1407: mol_type = other DNA
atgaataaagacacactaatacctacaactaaagatttaaaagtaaaaacaaatggtgaaaacattaatttaaagaactacaaggataattcttcatgtttcggcgtattcgaaaatgttgaaaatgctataagcagcgctgtacacgcacaaaagatattatcccttcattatacaaaagagcaacgtgaaaaaatcataactgagataagaaaggccgcattacaaaataaagaggtcttggctacaatgattctggaagaaacacatatgggacgttatgaggataaaatattaaaacatgaattggtagctaaatatactcctggtacagaagatttaactactactgcctggtcaggtgataatggtctgacagttgtagaaatgtctccatatggtgttattggtgcaataactccttctacgaatccaactgaaactgtaatatgtaatagcataggcatgattgctgctggaaatgctgtagtatttaacggacacccatgcgctaaaaaatgtgttgcctttgctgttgaaatgataaataaggcaattatttcatgtggcggtcctgaaaatctggtaacaactataaaaaatccaaccatggagtctctggatgcaattattaagcatccttcaataaaacttctttgcggaactgggggtccaggaatggtaaaaaccctgttaaattctggtaagaaagctataggtgctggtgctggaaatccaccagttattgtcgatgatactgctgatatagaaaaggctggtcgtagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatcgatttagtattacaaaaaaataatgaaactcaagaatactttataaacaaaaaatgggtaggaaaagatgcaaaattattcctcgatgaaatagatgttgagtctccttcaaatgttaaatgcataatctgcgaagtaaatgcaaatcatccatttgttatgacagaactgatgatgccaatattgccaattgtacgcgttaaagatatcgatgaagctattaaatatgcaaagatagcagaacaaaatagaaaacatagtgcctatatttattctaaaaatatcgacaacctgaatcgctttgaacgtgaaatagatactactatttttgtaaagaatgctaaatcttttgctggtgttggttatgaagcagaaggatttacaactttcactattgctggatctactggtgagggaataacctctgcacgtaattttacacgccaacgtcgctgtgtacttgccggctaa

SEQ ID NO: 72 - DNA, 1407 bp, synthetic construct
  misc_feature 1..1407: note = Description of Artificial Sequence: Synthetic polynucleotide
  source 1..1407: mol_type = other DNA
atgaataaagacacactgatccctacaactaaagatttaaaagtaaaaacaaatggtgaaaacattaatttaaagaactacaaagataatagcagttgtttcggcgtattcgaaaatgttgaaaatgctatcagcagcgctgtacacgcacaaaagatattatcgctgcattatacaaaagagcaacgtgaaaaaatcatcactgagatacgtaaggccgcattacaaaataaagaggtgctggctacaatgattctggaagaaacacatatgggacgttatgaggataaaatattaaaacatgaactggtagctaaatatactcctggtacagaagatttaactactactgcctggagcggtgataatggtctgacagttgtagaaatgtctccatatggtgttattggtgcaataactccttctaccaatccaactgaaactgtaatttgtaatagcattggcatgattgctgctggaaatgctgtagtatttaacggacacccatgcgctaaaaaatgtgttgcctttgctgttgaaatgatcaataaggcaattattagctgtggcggtccggaaaatctggtaacaactataaaaaatccaaccatggagtctctggatgccattattaagcatccttcaataaaactgctttgcggaactggcggtccaggaatggtaaaaaccctgttaaattctggtaagaaagctattggtgctggtgctggaaatccaccagttattgtcgatgatactgctgatattgaaaaggctggtcgtagcatcattgaaggctgttcttttgataataatttaccttgtattgcagaaaaagaagtatttgtttttgagaatgttgcagatgatttaatatctaacatgctgaaaaataatgctgtaattatcaatgaagatcaggtatcaaaattaatcgatttagtattacaaaaaaataatgaaactcaagaatactttatcaacaaaaaatgggtaggtaaagatgcaaaattattcctcgatgaaatcgatgttgagtctccttcaaatgttaaatgcattatctgcgaagtgaatgccaatcatccatttgttatgacagaactgatgatgccaatattgccaattgtgcgcgttaaagatatcgatgaagctattaaatatgcaaagattgcagaacaaaatagaaaacatagtgcctatatttatagcaaaaatatcgacaacctgaatcgctttgaacgtgaaatcgatactactatttttgtaaagaatgctaaatcttttgctggtgttggttatgaagcagaaggatttaccactttcactattgctggatctactggtgagggcataacctctgcacgtaattttacccgccaacgtcgctgtgtactggccggctaa

SEQ ID NO: 73 - DNA, 1407 bp, synthetic construct
  misc_feature 1..1407: note = Description of Artificial Sequence: Synthetic polynucleotide
  source 1..1407: mol_type = other DNA
atgaataaagacacgctgatcccgacaactaaagatctgaaagtaaaaaccaatggtgaaaacattaatctgaagaactacaaagataatagcagttgtttcggcgtattcgaaaatgttgaaaatgctatcagcagcgcggtacacgcacaaaagatactctcgctgcattataccaaagagcaacgtgaaaaaatcatcactgagatccgtaaggccgcattacaaaataaagaggtgctggcaacaatgattctggaagaaacacatatgggacgttatgaggataaaatactgaaacatgaactggtggcgaaatatacgcctggtactgaagatttaaccaccactgcctggagcggtgataatggtctgaccgttgtggaaatgtcgccttatggtgttattggtgcaattacgccttcaaccaatccaactgaaacggtaatttgtaatagcattggcatgattgctgctggaaatgcggtagtatttaacggtcacccctgcgctaaaaaatgtgttgcctttgctgttgaaatgatcaataaagcgattattagctgtggcggtccggaaaatctggtaaccactataaaaaatccaaccatggagtcgctggatgccattattaagcatccttcaatcaaactgctgtgcggcactggcggtccaggaatggtgaaaaccctgctgaatagcggtaagaaagcgattggtgctggtgctggaaatccaccagttattgtcgatgatactgctgatattgaaaaagcgggtcgtagcatcattgaaggctgttcttttgataataatttaccttgtattgcagaaaaagaagtatttgtttttgagaatgttgccgatgatctgatctctaacatgctgaaaaataatgcggtgattatcaatgaagatcaggttagcaaactgatcgatctggtattacaaaaaaataatgaaactcaagaatactttatcaacaaaaaatgggtaggtaaagatgcaaaactgttcctcgatgaaatcgatgttgagtcgccttcaaatgttaaatgcattatctgcgaagtgaatgccaatcatccatttgtgatgaccgaactgatgatgccaattttgccgattgtgcgcgttaaagatatcgatgaagcgattaaatatgcaaagattgcagaacaaaatcgtaaacatagtgcctatatttatagcaaaaatatcgacaacctgaatcgctttgaacgtgaaatcgataccactatttttgtgaagaatgctaaatcttttgctggtgttggttatgaagcagaaggttttaccactttcactattgctggaagcaccggtgaaggcattacctctgcacgtaattttacccgccaacgtcgctgtgtactggccggctaa

SEQ ID NO: 74 - DNA, 1407 bp, synthetic construct
  misc_feature 1..1407: note = Description of Artificial Sequence: Synthetic polynucleotide
  source 1..1407: mol_type = other DNA
atgaataaagatacgctgatcccgaccaccaaagatctgaaagtgaaaaccaacggcgaaaatatcaacctgaaaaactataaagataacagcagttgctttggcgtgtttgaaaacgttgaaaacgccatctccagcgcggtgcatgcgcaaaaaattctctcgctgcattacaccaaagagcagcgtgaaaaaattatcaccgaaatccgtaaagcggcgctgcaaaacaaagaagtgctggcaaccatgatcctggaagaaacgcatatggggcgttatgaagataaaattctgaaacatgaactggtggcgaaatacacgccgggcactgaagatctgaccaccaccgcctggagcggcgataacggcctgaccgtggtggagatgtcgccttatggcgtgattggcgcgattacgccgtcaaccaacccgaccgaaacggtgatttgtaacagcattggcatgattgccgcgggtaatgcggtggtgtttaacggtcatccctgcgcgaaaaaatgtgtggcgtttgccgttgagatgatcaacaaagcgattatcagctgcggcggcccggaaaatctggtgaccaccatcaaaaatccgaccatggaatcgctggatgccattatcaaacatccttccatcaaactgctgtgcggcaccggcggcccgggcatggtgaaaacgctgctgaacagcggtaaaaaagcgattggcgcgggcgcgggtaacccgccggtgattgtcgatgacaccgccgatattgaaaaagcggggcgtagcattattgaaggctgttcttttgataacaacctgccctgcattgccgaaaaagaagtgtttgtctttgaaaacgtcgccgatgatctgatcagcaatatgctgaaaaacaacgcggtgattatcaatgaagatcaggttagcaaactgatcgatctggtgctgcaaaaaaacaacgaaacgcaggaatattttatcaacaaaaaatgggttggtaaagatgccaaactgtttctcgatgaaatcgatgttgaatcgccgtctaacgtgaaatgtattatctgcgaagtgaacgccaaccatccgtttgtgatgaccgaactgatgatgccgattctgccgattgtgcgcgtgaaagatatcgatgaagcgattaaatatgccaaaattgccgaacaaaaccgtaaacacagcgcctatatttacagcaaaaatatcgataacctgaaccgctttgaacgtgaaatcgataccaccatttttgtgaaaaatgccaaaagttttgccggcgttggttatgaagcggaaggttttaccacctttaccattgccggtagcaccggcgaaggcattaccagcgcccgtaattttacccgccagcgtcgctgcgtgctggcgggctaa

SEQ ID NO: 75 - DNA, 1023 bp, Geobacillus thermoglucosidasius
  source 1..1023: mol_type = other DNA
atgaaagctgcagtagtagagcaatttaaggaaccattaaaaattaaagaagtggaaaagccatctatttcatatggcgaagtattagtccgcattaaagcatgcggtgtatgccatacggacttgcacgccgctcatggcgattggccagtaaaaccaaaacttcctttaatccctggccatgaaggagtcggaattgttgaagaagtcggtccgggggtaacccatttaaaagtgggagaccgcgttggaattccttggttatattctgcgtgcggccattgcgaatattgtttaagcggacaagaagcattatgtgaacatcaacaaaacgccggctactcagtcgacgggggttatgcagaatattgcagagctgcgccagattatgtggtgaaaattcctgacaacttatcgtttgaagaagctgctcctattttctgcgccggagttactacttataaagcgttaaaagtcacaggtacaaaaccgggagaatgggtagcgatctatggcatcggcggccttggacatgttgccgtccagtatgcgaaagcgatggggcttcatgttgttgcagtggatatcggcgatgagaaactggaacttgcaaaagagcttggcgccgatcttgttgtaaatcctgcaaaagaaaatgcggcccaatttatgaaagagaaagtcggcggagtacacgcggctgttgtgacagctgtatctaaacctgcttttcaatctgcgtacaattctatccgcagaggcggcacgtgcgtgcttgtcggattaccgccggaagaaatgcctattccaatctttgatacggtattaaacggaattaaaattatcggttccattgtcggcacgcggaaagacttgcaagaagcgcttcagttcgctgcagaaggtaaagtaaaaaccattattgaagtgcaacctcttgaaaaaattaacgaagtatttgacagaatgctaaaaggagaaattaacggacgggttgttttaacgttagaaaataataattaa

SEQ ID NO: 76 - protein, 340 aa, Geobacillus thermoglucosidasius
MKAAVVEQFKEPLKIKEVEKPSISYGEVLVRIKACGVCHTDLHAAHGDWPVKPKLPLIPGHEGVGIVEEVGPGVTHLKVGDRVGIPWLYSACGHCEYCLSGQEALCEHQQNAGYSVDGGYAEYCRAAPDYVVKIPDNLSFEEAAPIFCAGVTTYKALKVTGTKPGEWVAIYGIGGLGHVAVQYAKAMGLHVVAVDIGDEKLELAKELGADLVVNPAKENAAQFMKEKVGGVHAAVVTAVSKPAFQSAYNSIRRGGTCVLVGLPPEEMPIPIFDTVLNGIKIIGSIVGTRKDLQEALQFAAEGKVKTIIEVQPLEKINEVFDRMLKGEINGRVVLTLENNN

SEQ ID NO: 77 - DNA, 4090 bp, synthetic construct
  misc_feature 1..4090: note = Description of Artificial Sequence: Synthetic polynucleotide
  source 1..4090: mol_type = other DNA
atggctatcgaaatcaaagtaccggacatcggggctgatgaagttgaaatcaccgagatcctggtcaaagtgggcgacaaagttgaagccgaacagtcgctgatcaccgtagaaggcgacaaagcctctatggaagttccgtctccgcaggcgggtatcgttaaagagatcaaagtctctgttggcgataaaacccagaccggcgcactgattatgattttcgattccgccgacggtgcagcagacgctgcacctgctcaggcagaagagaagaaagaagcagctccggcagcagcaccagcggctgcggcggcaaaagacgttaacgttccggatatcggcagcgacgaagttgaagtgaccgaaatcctggtgaaagttggcgataaagttgaagctgaacagtcgctgatcaccgtagaaggcgacaaggcttctatggaagttccggctccgtttgctggcaccgtgaaagagatcaaagtgaacgtgggtgacaaagtgtctaccggctcgctgattatggtcttcgaagtcgcgggtgaagcaggcgcggcagctccggccgctaaacaggaagcagctccggcagcggcccctgcaccagcggctggcgtgaaagaagttaacgttccggatatcggcggtgacgaagttgaagtgactgaagtgatggtgaaagtgggcgacaaagttgccgctgaacagtcactgatcaccgtagaaggcgacaaagcttctatggaagttccggcgccgtttgcaggcgtcgtgaaggaactgaaagtcaacgttggcgataaagtgaaaactggctcgctgattatgatcttcgaagttgaaggcgcagcgcctgcggcagctcctgcgaaacaggaagcggcagcgccggcaccggcagcaaaagctgaagccccggcagcagcaccagctgcgaaagcggaaggcaaatctgaatttgctgaaaacgacgcttatgttcacgcgactccgctgatccgccgtctggcacgcgagtttggtgttaaccttgcgaaagtgaagggcactggccgtaaaggtcgtatcctgcgcgaagacgttcaggcttacgtgaaagaagctatcaaacgtgcagaagcagctccggcagcgactggcggtggtatccctggcatgctgccgtggccgaaggtggacttcagcaagtttggtgaaatcgaagaagtggaactgggccgcatccagaaaatctctggtgcgaacctgagccgtaactgggtaatgatcccgcatgttactcacttcgacaaaaccgatatcaccgagttggaagcgttccgtaaacagcagaacgaagaagcggcgaaacgtaagctggatgtgaagatcaccccggttgtcttcatcatgaaagccgttgctgcagctcttgagcagatgcctcgcttcaatagttcgctgtcggaagacggtcagcgtctgaccctgaagaaatacatcaacatcggtgtggcggtggataccccgaacggtctggttgttccggtattcaaagacgtcaacaagaaaggcatcatcgagctgtctcgcgagctgatgactatttctaagaaagcgcgtgacggtaagctgactgcgggcgaaatgcagggcggttgcttcaccatctccagcatcggcggcctgggtactacccacttcgcgccgattgtgaacgcgccggaagtggctatcctcggcgtttccaagtccgcgatggagccggtgtggaatggtaaagagttcgtgccgcgtctgatgctgccgatttctctctccttcgaccaccgcgtgatcgacggtgctgatggtgcccgtttcattaccatcattaacaacacgctgtctgacattcgccgtctggtgatgtaagtaaaagagccggcccaacggccggcttttttctggtaatctcatgaatgtattgaggttattagcgaatagacaaatcggttgccgtttgttgtttaaaaattgttaacaattttgtaaaataccgacggatagaacgacccggtggtggttagggtattacttcacataccctatggatttctgggtgcagcaaggtagcaagcgccagaatccccaggagcttacataagtaagtgactggggtgagggcgtgaagctaacgccgctgcggcctgaaagacgacgggtatgaccgccggagataaatatatagaggtcatgatgagtactgaaatcaaaactcaggtcgtggtacttggggcaggccccgcaggttactccgctgccttccgttgcgctgatttaggtctggaaaccgtaatcgtagaacgttacaacacccttggcggtgtttgtctgaacgtgggttgtatcccttctaaagcgctgctgcacgtggcaaaagttatcgaagaagcgaaagcgctggccgaacacggcatcgttttcggcgaaccgaaaactgacattgacaagatccgcacctggaaagaaaaagtcatcactcagctgaccggtggtctggctggcatggccaaaggtcgtaaagtgaaggtggttaacggtctgggtaaatttaccggcgctaacaccctggaagtggaaggcgaaaacggcaaaaccgtgatcaacttcgacaacgccatcatcgcggcgggttcccgtccgattcagctgccgtttatcccgcatgaagatccgcgcgtatgggactccaccgacgcgctggaactgaaatctgtaccgaaacgcatgctggtgatgggcggcggtatcatcggtctggaaatgggtaccgtataccatgcgctgggttcagagattgacgtggtggaaatgttcgaccaggttatcccggctgccgacaaagacgtggtgaaagtcttcaccaaacgcatcagcaagaaatttaacctgatgctggaagccaaagtgactgccgttgaagcgaaagaagacggtatttacgtttccatggaaggtaaaaaagcaccggcggaagcgcagcgttacgacgcagtgctggtcgctatcggccgcgtaccgaatggtaaaaacctcgatgcaggtaaagctggcgtggaagttgacgatcgcggcttcatccgcgttgacaaacaaatgcgcaccaacgtgccgcacatctttgctatcggcgatatcgtcggtcagccgatgctggcgcacaaaggtgtccatgaaggccacgttgccgcagaagttatctccggtctgaaacactacttcgatccgaaagtgatcccatccatcgcctacactaaaccagaagtggcatgggtcggtctgaccgagaaagaagcgaaagagaaaggcatcagctacgaaaccgccaccttcccgtgggctgcttccggccgtgctatcgcttctgactgcgcagatggtatgaccaaactgatcttcgacaaagagacccaccgtgttatcggcggcgcgattgtcggcaccaacggcggcgagctgctgggtgagatcggcctggctatcgagatgggctgtgacgctgaagacatcgccctgaccatccacgctcacccgactctgcacgagtccgttggcctggcggcggaagtgttcgaaggcagcatcaccgacctgccaaacgccaaagcgaagaaaaagtaactttttctttcaggaaaaaagcataagcggctccgggagccgctttttttatgcctgatgtttagaactatgtcactgttcataaaccgctacacctcatacatactttaagggcgaattctgcagatatccatcacactggcggccgctcgagcatgcatctagcacatccggcaattaaaaaagcggctaaccacgccgctttttttacgtctgcaatttacctttccagtcttcttgctccacgttcagagagacgttcgcatactgctgaccgttgctcgttattcagcctgacagtatggttactgtcgtttagacgttgtgggcggctctcctgaactttctcccgaaaaacctgacgttgttcaggtgatgccgattgaacacgctggcgggcgttatcacgttgctgttgattcagtgggcgctgctgtactttttcctt

SEQ ID NO: 78 - protein, 475 aa, Escherichia coli
MMSTEIKTQVVVLGAGPAGYSAAFRCADLGLETVIVERYNTLGGVCLNVGCIPSKALLHVAKVIEEAKALAEHGIVFGEPKTDIDKIRTWKEKVINQLTGGLAGMAKGRKVKVVNGLGKFTGANTLEVEGENGKTVINFDNAIIAAGSRPIQLPFIPHEDPRIWDSTDALELKEVPERLLVMGGGIIGLEMGTVYHALGSQIDVVEMFDQVIPAADKDIVKVFTKRISKKFNLMLETKVTAVEAKEDGIYVTMEGKKAPAEPQRYDAVLVAIGRVPNGKNLDAGKAGVEVDDRGFIRVDKQLRTNVPHIFAIGDIVGQPMLAHKGVHEGHVAAEVIAGKKHYFDPKVIPSIAYTEPEVAWVGLTEKEAKEKGISYETATFPWAASGRAIASDCADGMTKLIFDKESHRVIGGAIVGTNGGELLGEIGLAIEMGCDAEDIALTIHAHPTLHESVGLAAEVFEGSITDLPNPKAKKK

SEQ ID NO: 79 - protein, 475 aa, Klebsiella pneumoniae
MMSTEIKTQVVVLGAGPAGYSAAFRCADLGLETVIVERYSTLGGVCLNVGCIPSKALLHVAKVIEEAKALAEHGIVFGEPKTDIDKIRTWKEKVITQLTGGLAGMAKGRKVKVVNGLGKFTGANTLEVEGENGKTVINFDNAIIAAGSRPIQLPFIPHEDPRVWDSTDALELKSVPKRMLVMGGGIIGLEMGTVYHALGSEIDVVEMFDQVIPAADKDVVKVFTKRISKKFNLMLEAKVTAVEAKEDGIYVSMEGKKAPAEAQRYDAVLVAIGRVPNGKNLDAGKAGVEVDDRGFIRVDKQMRTNVPHIFAIGDIVGQPMLAHKGVHEGHVAAEVISGLKHYFDPKVIPSIAYTKPEVAWVGLTEKEAKEKGISYETATFPWAASGRAIASDCADGMTKLIFDKETHRVIGGAIVGTNGGELLGEIGLAIEMGCDAEDIALTIHAHPTLHESVGLAAEVFEGSITDLPNAKAKKK

SEQ ID NO: 80 - DNA, 347 bp, synthetic construct
  misc_feature 1..347: note = Description of Artificial Sequence: Synthetic polynucleotide
  source 1..347: mol_type = other DNA
ataataatacatatgaaccatgcgagttacgggcctataagccaggcgagatatgatctatatcaatttctcatctataatgctttgttagtatctcgtcgccgacttaataaagagagagttagtgtgaaagctgacaacccttttgatcttttacttcctgctgcaatggccaaagtggccgaagaggcgggtgtctataaagcaacgaaacatccgcttaagactttctatctggcgattaccgccggtgttttcatctcaatcgcattcaccactggcacaggcacagaaggtaggtgttacatgtcagaacgtttacacaatgacgtggatcctattattat

SEQ ID NO: 81 - DNA, 4678 bp, synthetic construct
  misc_feature 1..4678: note = Description of Artificial Sequence: Synthetic polynucleotide
  source 1..4678: mol_type = other DNA
aagaggtaaaagaataatggctatcgaaatcaaagtaccggacatcggggctgatgaagttgaaatcaccgagatcctggtcaaagtgggcgacaaagttgaagccgaacagtcgctgatcaccgtagaaggcgacaaagcctctatggaagttccgtctccgcaggcgggtatcgttaaagagatcaaagtctctgttggcgataaaacccagaccggcgcactgattatgattttcgattccgccgacggtgcagcagacgctgcacctgctcaggcagaagagaagaaagaagcagctccggcagcagcaccagcggctgcggcggcaaaagacgttaacgttccggatatcggcagcgacgaagttgaagtgaccgaaatcctggtgaaagttggcgataaagttgaagctgaacagtcgctgatcaccgtagaaggcgacaaggcttctatggaagttccggctccgtttgctggcaccgtgaaagagatcaaagtgaacgtgggtgacaaagtgtctaccggctcgctgattatggtcttcgaagtcgcgggtgaagcaggcgcggcagctccggccgctaaacaggaagcagctccggcagcggcccctgcaccagcggctggcgtgaaagaagttaacgttccggatatcggcggtgacgaagttgaagtgactgaagtgatggtgaaagtgggcgacaaagttgccgctgaacagtcactgatcaccgtagaaggcgacaaagcttctatggaagttccggcgccgtttgcaggcgtcgtgaaggaactgaaagtcaacgttggcgataaagtgaaaactggctcgctgattatgatcttcgaagttgaaggcgcagcgcctgcggcagctcctgcgaaacaggaagcggcagcgccggcaccggcagcaaaagctgaagccccggcagcagcaccagctgcgaaagcggaaggcaaatctgaatttgctgaaaacgacgcttatgttcacgcgactccgctgatccgccgtctggcacgcgagtttggtgttaaccttgcgaaagtgaagggcactggccgtaaaggtcgtatcctgcgcgaagacgttcaggcttacgtgaaagaagctatcaaacgtgcagaagcagctccggcagcgactggcggtggtatccctggcatgctgccgtggccgaaggtggacttcagcaagtttggtgaaatcgaagaagtggaactgggccgcatccagaaaatctctggtgcgaacctgagccgtaactgggtaatgatcccgcatgttactcacttcgacaaaaccgatatcaccgagttggaagcgttccgtaaacagcagaacgaagaagcggcgaaacgtaagctggatgtgaagatcaccccggttgtcttcatcatgaaagccgttgctgcagctcttgagcagatgcctcgcttcaatagttcgctgtcggaagacggtcagcgtctgaccctgaagaaatacatcaacatcggtgtggcggtggataccccgaacggtctggttgttccggtattcaaagacgtcaacaagaaaggcatcatcgagctgtctcgcgagctgatgactatttctaagaaagcgcgtgacggtaagctgactgcgggcgaaatgcagggcggttgcttcaccatctccagcatcggcggcctgggtactacccacttcgcgccgattgtgaacgcgccggaagtggctatcctcggcgtttccaagtccgcgatggagccggtgtggaatggtaaagagttcgtgccgcgtctgatgctgccgatttctctctccttcgaccaccgcgtgatcgacggtgctgatggtgcccgtttcattaccatcattaacaacacgctgtctgacattcgccgtctggtgatgtaagtaaaagagccggcccaacggccggcttttttctggtaatctcatgaatgtattgaggttattagcgaatagacaaatcggttgccgtttgttaagccaggcgagatatgatctatatcaatttctcatctataatgctttgttagtatctcgtcgccgacttaataaagagagagttagtcttctatatcacagcaagaaggtaggtgttacatgatgagtactgaaatcaaaactcaggtcgtggtacttggggcaggccccgcaggttactctgcagccttccgttgcgctgatttaggtctggaaaccgtcatcgtagaacgttacagcaccctcggtggtgtttgtctgaacgtgggttgtatcccttctaaagcgctgctgcacgtggcaaaagttatcgaagaagcgaaagcgctggccgaacacggcatcgttttcggcgaaccgaaaactgacattgacaagatccgcacctggaaagaaaaagtcatcactcagctgaccggtggtctggctggcatggccaaaggtcgtaaagtgaaggtggttaacggtctgggtaaatttaccggcgctaacaccctggaagtggaaggcgaaaacggcaaaaccgtgatcaacttcgacaacgccatcatcgcggcgggttcccgtccgattcagctgccgtttatcccgcatgaagatccgcgcgtatgggactccaccgacgcgctggaactgaaatctgtaccgaaacgcatgctggtgatgggcggcggtatcatcggtctggaaatgggtaccgtataccatgcgctgggttcagagattgacgtggtggaaatgttcgaccaggttatcccggctgccgacaaagacgtggtgaaagtcttcaccaaacgcatcagcaagaaatttaacctgatgctggaagccaaagtgactgccgttgaagcgaaagaagacggtatttacgtttccatggaaggtaaaaaagcaccggcggaagcgcagcgttacgacgcagtgctggtcgctatcggccgcgtaccgaatggtaaaaacctcgatgcaggtaaagctggcgtggaagttgacgatcgcggcttcatccgcgttgacaaacaaatgcgcaccaacgtgccgcacatctttgctatcggcgatatcgtcggtcagccgatgctggcgcacaaaggtgtccatgaaggccacgttgccgcagaagttatctccggtctgaaacactacttcgatccgaaagtgatcccatccatcgcctacactaaaccagaagtggcatgggtcggtctgaccgagaaagaagcgaaagagaaaggcatcagctacgaaaccgccaccttcccgtgggctgcttccggccgtgctatcgcttctgactgcgcagatggtatgaccaaactgatcttcgacaaagagacccaccgtgttatcggcggcgcgattgtcggcaccaacggcggcgagctgctgggtgagatcggcctggctatcgagatgggctgtgacgctgaagacatcgccctgaccatccacgctcacccgactctgcacgagtccgttggcctggcggcggaagtgttcgaaggcagcatcaccgacctgccaaacgccaaagcgaagaaaaagtaactttttctttcaggaaaaaagcataagcggctccgggagccgctttttttatgcctgatgtttagaactatgtcactgttcataaaccgctacacctcatacatactttaagggcgaattctgcagatatccatcacactggcggccgctcgagcatgcatctagcacatccggcaattaaaaaagcggctaaccacgccgctttttttacgtctgcaatttacctttccagtcttcttgctccacgttcagagagacgttcgcatactgctgaccgttgctcgttattcagcctgacagtatggttactgtcgtttagacgttgtgggcggctctcctgaactttctcccgaaaaacctgacgttgttcaggtgatgccgattgaacacgctggcgggcgttatcacgttgctgttgattcagtgggcgctgctgtactttttccttaaacacctggcgctgctctggtgatgcggactgaatacgctcacgcgctgcgtctcttcgctgctggttctgcgggttagtctgcattttctcgcgaaccgcctggcgctgctcaggcgaggcggactgaatgcgctcacgcgctgcctctcttcgctgctggatcttcgggttagtctgcattctctcgcgaactgcctggcgctgctcaggcgaggcggactgataacgctgacgagcggcgtccttttgttgctgggtcagtggttggcgacggctgaagtcgtggaagtcgtcatagctcccatagtgttcagcttcattaaaccgctgtgccgctgcctgacgttgggtacctcgtgtaatgactggtgcggcgtgtgttcgttgctgaaactgatttgctgccgcctgacgctggctgtcgcgcgttggggcaggtaattgcgtggcgctcattccgccgttgacatcggtttgatgaaaccgctttgccatatcctgatcatgatagggcacaccattacggtagtttggattgtgccgccatgccatattcttatcagtaagatgctcaccggtgatacggttgaaattgttgacgtcgatattgatgttgtcgccgttgtgttgccagccattaccgtcacgatgaccgccatcgtggtgatgataatcat

SEQ ID NO: 82 - DNA, 1114 bp, synthetic construct
  misc_feature 1..1114: note = Description of Artificial Sequence: Synthetic polynucleotide
  source 1..1114: mol_type = other DNA
  CDS 323..958
caaaaaaccggagtctgtgctccggttttttattatccgctaatcaattacatatgaatatcctccttagttcctattccgaagttcctattctctagaaagtataggaacttcggcgcgcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttcgaagcagctccagcctacacccttcttcagggctgactgtttgcataaaaattcatctgtatgcacaata

SEQ ID NO: 83 - protein, 212 aa, synthetic construct
  REGION 1..212: note = Description of Artificial Sequence: Synthetic polypeptide
MEKKITGYTTVDISQWHRKEHFEAFQSVAQCTYNQTVQLDITAFLKTVKKNKHKFYPAFIHILARLMNAHPELRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRCLMNTTVLR

SEQ ID NO: 84 - DNA, 2521 bp, synthetic construct
  misc_feature 1..2521: note = Description of Artificial Sequence: Synthetic polynucleotide
  source 1..2521: mol_type = other DNA
ttatttggtgatattggtaccaatatcatgcagcaaacggtgcaacattgccgtgtctcgttgctctaaaagccccaggcgttgttgtaaccagtcgaccagttttatgtcatctgccactgccagagtcgtcagcaatgtcatggctcgttcgcgtaaagcttgcagttgatgttggtctgccgttgcatcacttttcgccggttgttgtattaatgttgctaattgatagcaatagaccatcaccgcctgccccagattgagcgaaggataatccgccaccatcggcacaccagtaagaacgtcagccaacgctaactcttcgttagtcaacccggaatcttcgcgaccaaacaccagcgcggcatggctcatccatgaagatttttcctctaacagcggcaccagttcaactggcgtggcgtagtaatgatatttcgcccgactgcgcgcagtggtggcgacagtgaaatcgacatcgtgtaacgattcagccaatgtcgggaaaactttaatattatcaataatatcaccagatccatgtgcgacccagcgggtggctggctccaggtgtgcctgactatcgacaatccgcagatcgctaaaccccatcgttttcattgcccgcgccgctgccccaatattttctgctctggcgggtgcgaccagaataatcgttatacgcatattgccactcttcttgatcaaataaccgcgaaccgggtgatcactgtcaacttattacgcggtgcgaatttacaaattcttaacgtaagtcgcagaaaaagccctttacttagcttaaaaaaggctaaactatttcctgactgtactaacggttgagttgttaaaaaatgctacatatccttctgtttacttaggataattttataaaaaataaatctcgacaattggattcaccacgtttattagttgtatgatgcaactagttggattattaaaataatgtgacgaaagctagcatttagatacgatgatttcatcaaactgttaacgtgctacaattgaacttgatatatgtcaacgaagcgtagttttattgggtgtccggcccctcttagcctgttatgttgctgttaaaatggttaggatgacagccgtttttgacactgtcgggtcctgagggaaagtacccacgaccaagctaatgatgttgttgacgttgatggaaagtgcatcaagaacgcaattacgtactttagtcatgttacgccgatcatgttaatttgcagcatgcatcaggcaggtcagggacttttgtacttcctgtttcgatttagttggcaatttaggtagcaaacgaattcatcggctttaccaccgtcaaaaaaaacggcgctttttagcgccgtttttatttttcaaccttatttccagatacgtaactcatcgtccgttgtaacttctttactggctttcattttcggcagtgaaaacgcataccagtcgatattacgggtcacaaacatcatgccggccagcgccaccaccagcacactggttcccaacaacagcgcgctatcggcagagttgagcagtccccacatcacaccatccagcaacaacagcgcgagggtaaacaacatgctgttgcaccaacctttcaataccgcttgcaaataaataccgttcattatcgccccaatcagactggcgattatccatgccacggtaaaaccggtatgttcagaaagcgccagcaagagcaaataaaacatcaccaatgaaagccccaccagcaaatattgcattgggtgtaaacgttgcgcggtgagcgtttcaaaaacaaagaacgccataaaagtcagtgcaatcagcagaatggcgtacttagtcgcccggtcagttaattggtattgatcggctggcgtcgttactgcgacgctaaacgccgggaagttttcccagccggtatcattgcctgaagcaaaacgctcaccgagattattagcaaaccagctgctttgccagtgcgcctgaaaacctgactcgctaacttcccgtttggctggtagaaaatcacctaaaaaactgggatgcggccagttgctggttaaggtcatttcgctattacgcccgccaggcaccacagaaagatcgccggtaccgcttaaattcagggccatattcagcttcaggttctgcttccgccagtccccttcaggtaaagggatatgcacgccctgcccgccttgctctaacccggtgccgggttcaatggtcagcgccgttccgttaacttcaggcgctttcaccacaccaataccacgcgcatccccgacgctaatcacaataaatggcttgcctaaggtgatatttggcgcgttgagttcgctaagacgcgaaacatcgaaatcggcttttaacgttaaatcactgtgccagacctgaccggtataaatccctatcttgcgttcttccacgttctgattgccatcaaccatcaatgactcaggtaaccaaaaatggataaaacttcgtttccgctgcagggttttat

SEQ ID NO: 85 - DNA, 3010 bp, synthetic construct
  misc_feature 1..3010: note = Description of Artificial Sequence: Synthetic polynucleotide
  source 1..3010: mol_type = other DNA
aagccacagcaggatgcccactgcaacaaaggtgatcacaccggaaacgcgatggagaatggacgctatcgccgtgatggggaaccggatggtctgtaggtccagattaacaggtctttgttttttcacatttcttatcatgaataacgcccacatgctgttcttattattccctggggactacgggcacagaggttaactttctgttacctggagacgtcgggatttccttcctccggtctgcttgcgggtcagacagcgtcctttctataactgcgcgtcatgcaaaacactgcttccagatgcgaaaacgacacgttacaacgctgggtggctcgggattgcagggtgttccggagacctggcggcagtataggctgttcacaaaatcattacaattaacctacatatagtttgtcgggttttatcctgaacagtgatccaggtcacgataacaacatttatttaatttttaatcatctaatttgacaatcattcaacaaagttgttacaaacattaccaggaaaagcatataatgcgtaaaagttatgaagtcggtatttcacctaagattaacttatgtaacagtgtggaagtattgaccaattcattcgggacagttattagtggtagacaagtttaataattcggattgctaagtacttgattcgccatttattcgtcatcaatggatcctttacctgcaagcgcccagagctctgtacccaggttttcccctctttcacagagcggcgagccaaataaaaaacgggtaaagccaggttgatgtgcgaaggcaaatttaagttccggcagtcttacgcaataaggcgctaaggagaccttaaatggctgatacaaaagcaaaactcaccctcaacggggatacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccatgctttccgtcgcgactcgcatccaatggcagtcatgtgtggtattaccggcgcgctggcggcgttctatcacgactcgctggatgttaacaatcctcgtcaccgtgaaattgccgcgttcctcctgctgtcgaaaatgccgaccatggccgcgatgtgttacaagtattccattggtcagccatttgtttacccgcgcaacgatctctcctacgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggcggtgctaacgaagcggcgctgaaaatgctggaagaaatcagctccgttaaacacattccggaatttgttcgtcgtgcgaaagacaaaaatgattctttccgcctgatgggcttcggtcaccgcgtgtacaaaaattacgacccgcgcgccaccgtaatgcgtgaaacctgccatgaagtgctgaaagagctgggcacgaaggatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaatggttgattgctaagttgtaaatattttaacccgccgttcatatggcgggttgatttttatatgcctaaacacaaaaaattgtaaaaataaaatccattaacagacctatatagatatttaaaaagaatagaacagctcaaattatcagcaacccaatactttcaattaaaaacttcatggtagtcgcatttataaccctatgaaaatgacgtctatctatacccccctatattttattcatcatacaacaaattcatgataccaataatttagttttgcatttaataaaactaacaatatttttaagcaaaactaaaaactagcaataatcaaatacgatattctggcgtagctatacccctattctatatccttaaaggactctgttatgtttaaaggacaaaaaacattggccgcactggccgtatctctgctgttcactgcacctgtttatgctgctgatgaaggttctggcgaaattcactttaagggggaggttattgaagcaccttgtgaaattcatccagaagatattgataaaaacatagatcttggacaagtcacgacaacccatataaaccgggagcatcatagcaataaagtggccgtcgacattcgcttgatcaactgtgatctgcctgcttctgacaacggtagcggaatgccggtatccaaagttggcgtaaccttcgatagcacggctaagacaactggtgctacgcctttgttgagcaacaccagtgcaggcgaagcaactggggtcggtgtacgactgatggacaaaaatgacggtaacatcgtattaggttcagccgcgccagatcttgacctggatgcaagctcatcagaacagacgctgaactttttcgcctggat

SEQ ID NO: 86 - DNA, 4180 bp, synthetic construct
  misc_feature 1..4180: note = Description of Artificial Sequence: Synthetic polynucleotide
  source 1..4180: mol_type = other DNA
cgcgatgtcgacgtcacgaaactgaaaaaaccgctctacattctggcgactgctgatgaagaaaccagtatggccggagcgcgttattttgccgaaactaccgccctgcgcccggattgcgccatcattggcgaaccgacgtcactacaaccggtacgcgcacataaaggtcatatctctaacgccatccgtattcagggccagtcggggcactccagcgatccagcacgcggagttaacgctatcgaactaatgcacgacgccatcgggcatattttgcaattgcgcgataacctgaaagaacgttatcactacgaagcgtttaccgtgccataccctacgctcaacctcgggcatattcacggtggcgacgcttctaaccgtatttgcgcttgctgtgagttgcatatggatattcgtccgctgcctggcatgacactcaatgaacttaatggtttgctcaacgatgcattggctccggtgagcgaacgctggccgggtcgtctgacggtcgacgagctgcatccgccgatccctggctatgaatgcccaccgaatcatcaactggttgaagtggttgagaaattgctcggagcaaaaaccgaagtggtgaactactgtaccgaagcgccgtttattcaaacgttatgcccgacgctggtgttggggcctggctcaattaatcaggctcatcaacctgatgaatatctggaaacacggtttatcaagcccacccgcgaactgataacccaggtaattcaccatttttgctggcattaaaacgtaggccggataaggcgctcgcgccgcatccggcgctgttgccaaactccagtgccgcaataatgtcggatgcgatgcttgcgcatcttatccgacctacagtgactcaaacgatgcccaaccgtaggccggataaggcgctcgcgccgcatccggcactgttgccaaactccagtgccgcaataatgtcggatgcgatacttgcgcatcttatccgaccgacagtgactcaaacgatgcccaactgtaggccggataaggcgctcgcgccgcatccggcactgttgccaaactccagtgccgcaataatgtcggatgcgatacttgcgcatcttatccgacctacacctttggtgttacttggggcgattttttaacatttccataagttacgcttatttaaagcgtcgtgaatttaatgacgtaaattcctgctatttattcgtttgctgaagcgatttcgcagcatttgacgtcaccgcttttacgtggctttataaaagacgacgaaaagcaaagcccgagcatattcgcgccaatgctagcaagaggagaagtcgacatgacagacttaaataaagtggtaaaagaacttgaagctcttggtatttatgacgtaaaagaagttgtttacaatccaagctacgagcaattgttcgaagaagaaactaaaccaggtttagaaggctttgaaaaaggtactttaactacgactggtgcagtggcagtagatacaggtatcttcacaggtcgttctccaaaagataaatatatcgtgttagatgaaaaaaccaaagatactgtttggtggacatctgaaacagcaaaaaacgacaacaagccaatgaaccaagctacatggcaaagcttaaaagacttggtaaccaaccagctttctcgtaaacgcttatttgtagttgatggtttctgtggtgcgagcgaacacgaccgtattgcagtacgtattgtcactgaagtagcgtggcaagcacattttgtaaaaaatatgtttattcgcccaactgaagaacaactcaaaaattttgaaccagatttcgttgtaatgaatggttctaaagtaaccaatccaaactggaaagaacaaggtttaaattcagaaaactttgttgctttcaacttgactgaacgcattcaattaatcggtggtacttggtacggcggtgaaatgaaaaaaggtatgttctcaatcatgaactacttcctaccacttaaaggtgttggtgcaatgcactgctcagctaacgttggtaaagatggcgatgtagcaatcttcttcggcttatctggcacaggtaaaacaaccctttcaacggatccaaaacgtgaattaatcggtgacgatgaacacggctgggatgatgtgggtatctttaactttgaaggtggttgctatgcgaaaaccattcacctttcagaagaaaatgaaccagatatttaccgcgctatccgtcgcgacgcattattagaaaacgtggttgttcgtgcagatggttctgttgatttcgatgatggttcaaaaacagaaaatactcgcgtgtcttacccaatttatcacattgataacattgtaaaaccagtttctcgtgcaggtcacgcaactaaagtgattttcttaactgcagatgcatttggcgtattaccaccagtatctaaattgacaccagaacaaactaaatactacttcttatctggtttcacagcaaaattagcaggtactgaacgtggtattactgaaccaactccaactttctcagcatgtttcggtgctgcgttcttaacccttcacccaactcaatatgcagaagtgttagtaaaacgtatgcaagcagtgggtgctgaagcttacttagtaaatactggttggaatggcacaggcaaacgtatctcaatcaaagatactcgcggaatcattgatgcaatcttagatggctcaattgaaaaagctgaaatgggcgaattaccaatctttaacttagccattcctaaagcattaccaggtgtagattctgcaatcttagatcctcgcgatacttacgcagataaagcacaatggcaatcaaaagctgaagacttagcaggtcgttttgtgaaaaactttgttaaatatgcaactaacgaagaaggcaaagctttaattgcagctggtcctaaagcttaatctagaaagcttcctagaggcatcaaataaaacgaaaggctcagtcgaaagactgggcctttcgttttatctgttgtttgtcggtgaacgctctcctgagtaggacgaattcacttctgttctaacaccctcgttttcaatatatttctgtctgcattttattcaaattctgaatataccttcagatatccttaaggaattgtcgttacattcggcgatattttttcaagacaggttcttactatgcattccacagaagtccaggctaaacctctttttagctggaaagccctgggttgggcactgctctacttttggtttttctctactctgctacaggccattatttacatcagtggttatagtggcactaacggcattcgcgactcgctgttattcagttcgctgtggttgatcccggtattcctctttccgaagcggattaaaattattgccgcagtaatcggcgtggtgctatgggcggcctctctggcggcgctgtgctactacgtcatctacggtcaggagttctcgcagagcgttctgtttgtgatgttcgaaaccaacaccaacgaagccagcgagtatttaagccagtatttcagcctgaaaattgtgcttatcgcgctggcctatacggcggtggcagttctgctgtggacacgcctgcgcccggtctatattccaaagccgtggcgttatgttgtctcttttgccctgctttatggcttgattctgcatccgatcgccatgaatacgtttatcaaaaacaagccgtttgagaaaacgttggataacctggcctcgcgtatggagcctgccgcaccgtggcaattcctgaccggctattatcagtatcgtcagcaactaaactcgctaacaaagttactgaatgaaaataatgccttgccgccactggctaatttcaaagatgaatcgggtaacgaaccgcgcactttagtgctggtgattggcgagtcgacccagcgcggacgcatgagtctgtacggttatccgcgtgaaaccacgccggagctggatgcgctgcataaaaccgatccgaatctgaccgtgtttaataacgtagttacgtctcgtccgtacaccattgaaatcctgcaacaggcgctgacctttgccaatgaaaagaacccggatctgtatctgacgcagccgtcgctgatgaacatgatgaaacaggcgggttataaaaccttc

SEQ ID NO: 87 - DNA, 4960 bp, synthetic construct
  misc_feature 1..4960: note = Description of Artificial Sequence: Synthetic polynucleotide
  source 1..4960: mol_type = other DNA
aataggcgtatcacgaggccctttcgtcttcacctcgagaattgtgagcggataacaattgacattgtgagcggataacaagatactgagcacatcagcaggacgcactgaccgaattcaattaagctagcaagaggagaagtcgagatgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataatgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataatctagcaagaggagaagtcgacatggaaatcaaagaaatggtgagccttgcacgcaaggctcagaaggagtatcaagctacccataaccaagaagcagttgacaacatttgccgagctgcagcaaaagttatttatgaaaatgcagctattctggctcgcgaagcagtagacgaaaccggcatgggcgtttacgaacacaaagtggccaagaatcaaggcaaatccaaaggtgtttggtacaacctccacaataaaaaatcgattggtatcctcaatatagacgagcgtaccggtatgatcgagattgcaaagcctatcggagttgtaggagccgtaacgccgacgaccaacccgatcgttactccgatgagcaatatcatctttgctcttaagacctgcaatgccatcattattgccccccaccccagatccaaaaaatgctctgcacacgcagttcgtctgatcaaagaagctatcgctccgttcaacgtaccggaaggtatggttcagatcatcgaagaacccagcatcgagaagacgcaggaactcatgggcgccgtagacgtagtagttgctacgggtggtatgggcatggtgaagtctgcatattcttcaggaaagccttctttcggtgttggagccggtaacgttcaggtgatcgtggatagcaacatcgatttcgaagctgctgcagaaaaaatcatcaccggtcgtgctttcgacaacggtatcatctgctcaggcgaacagagcatcatctacaacgaggctgacaaggaagcagttttcacagcattccgcaaccacggtgcatatttctgtgacgaagccgaaggagatcgggctcgtgcagctatcttcgaaaatggagccatcgcgaaagatgtagtaggtcagagcgttgccttcattgccaagaaagcaaacatcaatatccccgagggtacccgtattctcgttgttgaagctcgcggcgtaggagcagaagacgttatctgtaaggaaaagatgtgtcccgtaatgtgcgccctcagctacaagcacttcgaagaaggtgtagaaatcgcacgtacgaacctcgccaacgaaggtaacggccacacctgtgctatccactccaacaatcaggcacacatcatcctcgcaggatcagagctgacggtatctcgtatcgtagtgaatgctccgagtgccactacagcaggcggtcacatccaaaacggtcttgccgtaaccaatacgctcggatgcggatcatggggtaataactctatctccgagaacttcacttacaagcacctcctcaacatttcacgcatcgcaccgttgaattcaagcattcacatccccgatgacaaagaaatctgggaactctaatctagcaagaggagaagtcgacatgcaacttttcaaactcaagagtgtaacacatcactttgacacttttgcagaatttgccaaggaattctgtcttggagaacgcgacttggtaattaccaacgagttcatctatgaaccgtatatgaaggcatgccagctcccctgccattttgttatgcaggagaaatatgggcaaggcgagccttctgacgaaatgatgaataacatcttggcagacatccgtaatatccagttcgaccgcgtaatcggtatcggaggaggtacggttattgacatctctaaacttttcgttctgaaaggattaaatgatgtactcgatgcattcgaccgcaaaatacctcttatcaaagagaaagaactgatcattgtgcccacaacatgcggaacgggtagcgaggtgacgaacatttctatcgcagaaatcaaaagccgtcacaccaaaatgggattggctgacgatgccattgttgcagaccatgccatcatcatacctgaacttctgaagagcttgcctttccacttctacgcatgcagtgcaatcgatgctcttatccatgccatcgagtcatacgtatctcctaaagccagtccatattctcgtctgttcagtgaggcggcttgggacattatcctggaagtattcaagaaaatcgccgaacacggccctgaataccgcttcgaaaagctgggagaaatgatcatggccagcaactatgccggtatagccttcggaaatgcaggagtaggagccgtccacgcactatcctacccgttgggaggcaactatcacgtgccgcatggagaagcaaactatcagttcttcacagaggtattcaaagtataccaaaagaagaatcctttcggctatatagtcgaactcaactggaagctctccaagatactgaactgccagcccgaatacgtatatccgaagctggatgaacttctcggatgccttcttaccaagaaacctttgcacgaatacggcatgaaggacgaagaggtaagaggctttgcggaatcagtgcttaagacacagcaaagattgctcgccaacaactacgtagagcttactgtagatgagatcgaaggtatctacagaagactctactaatctagaaagcttcctagaggcatcaaataaaacgaaaggctcagtcgaaagactgggcctttcgttttatctgttgtttgtcggtgaacgctctcctgagtaggacaaatccgccgccctagacctaggcgttcggctgcgacacgtcttgagcgattgtgtaggctggagctgcttcgaagttcctatactttctagagaataggaacttcggaataggaactaaggaggatattcatatggaccatggctaattcccat

SEQ ID NO: 88 - DNA, 5083 bp, synthetic construct
  misc_feature 1..5083: note = Description of Artificial Sequence: Synthetic polynucleotide
  source 1..5083: mol_type = other DNA
tcgagaaatttatcaaaaagagtgttgacttgtgagcggataacaatgatacttagattcaattgtgagcggataacaatttcacacagaattcaattaagctagcaagaggagaagtcgacatggccaacataagttcaccattcgggcaaaacgaatggctggttgaagagatgtaccgcaagttccgcgacgacccctcctcggtcgatcccagctggcacgagttcctggttgactacagccccgaacccacctcccaaccagctgccgaaccaacccgggttacctcgccactcgttgccgagcgggccgctgcggccgccccgcaggcaccccccaagccggccgacaccgcggccgcgggcaacggcgtggtcgccgcactggccgccaaaactgccgttcccccgccagccgaaggtgacgaggtagcggtgctgcgcggcgccgccgcggccgtcgtcaagaacatgtccgcgtcgttggaggtgccgacggcgaccagcgtccgggcggtcccggccaagctactgatcgacaaccggatcgtcatcaacaaccagttgaagcggacccgcggcggcaagatctcgttcacgcatttgctgggctacgccctggtgcaggcggtgaagaaattcccgaacatgaaccggcactacaccgaagtcgacggcaagcccaccgcggtcacgccggcgcacaccaatctcggcctggcgatcgacctgcaaggcaaggacgggaagcgttccctggtggtggccggcatcaagcggtgcgagaccatgcgattcgcgcagttcgtcacggcctacgaagacatcgtacgccgggcccgcgacggcaagctgaccactgaagactttgccggcgtgacgatttcgctgaccaatcccggaaccatcggcaccgtgcattcggtgccgcggctgatgcccggccagggcgccatcatcggcgtgggcgccatggaataccccgccgagtttcaaggcgccagcgaggaacgcatcgccgagctgggcatcggcaaattgatcactttgacctccacctacgaccaccgcatcatccagggcgcggaatcgggcgacttcctgcgcaccatccacgagttgctgctctcggatggcttctgggacgaggtcttccgcgaactgagcatcccatatctgccggtgcgctggagcaccgacaaccccgactcgatcgtcgacaagaacgctcgcgtcatgaacttgatcgcggcctaccgcaaccgcggccatctgatggccgataccgacccgctgcggttggacaaagctcggttccgcagtcaccccgacctcgaagtgctgacccacggcctgacgctgtgggatctcgatcgggtgttcaaggtcgacggctttgccggtgcgcagtacaagaaactgcgcgacgtgctgggcttgctgcgcgatgcctactgccgccacatcggcgtggagtacgcccatatcctcgaccccgaacaaaaggagtggctcgaacaacgggtcgagaccaagcacgtcaaacccactgtggcccaacagaaatacatcctcagcaagctcaacgccgccgaggcctttgaaacgttcctacagaccaagtacgtcggccagaagcggttctcgctggaaggcgccgaaagcgtgatcccgatgatggacgcggcgatcgaccagtgcgctgagcacggcctcgacgaggtggtcatcgggatgccgcaccggggccggctcaacgtgctggccaacatcgtcggcaagccgtactcgcagatcttcaccgagttcgagggcaacctgaatccgtcgcaggcgcacggctccggtgacgtcaagtaccacctgggcgccaccgggctgtacctgcagatgttcggcgacaacgacattcaggtgtcgctgaccgccaacccgtcgcatctggaggccgtcgacccggtgctggagggattggtgcgggccaagcaggatctgctcgaccacggaagcatcgacagcgacggccaacgggcgttctcggtggtgccgctgatgttgcatggcgatgccgcgttcgccggtcagggtgtggtcgccgagacgctgaacctggcgaatctgccgggctaccgcgtcggcggcaccatccacatcatcgtcaacaaccagatcggcttcaccaccgcgcccgagtattccaggtccagcgagtactgcaccgacgtcgcaaagatgatcggggcaccgatctttcacgtcaacggcgacgacccggaggcgtgtgtctgggtggcgcggttggcggtggacttccgacaacggttcaagaaggacgtcgtcatcgacatgctgtgctaccgccgccgcgggcacaacgagggtgacgacccgtcgatgaccaacccctacatgtacgacgtcgtcgacaccaagcgcggggcccgcaaaagctacaccgaagccctgatcggacgtggcgacatctcgatgaaggaggccgaggacgcgctgcgcgactaccagggccagctggaacgggtgttcaacgaagtgcgcgagctggagaagcacggtgtgcagccgagcgagtcggtcgagtccgaccagatgattcccgcggggctggccactgcggtggacaagtcgctgctggcccggatcggcgatgcgttcctcgccttgccgaacggcttcaccgcgcacccgcgagtccaaccggtgctggagaagcgccgggagatggcctatgaaggcaagatcgactgggcctttggcgagctgctggcgctgggctcgctggtggccgaaggcaagctggtgcgcttgtcggggcaggacagccgccgcggcaccttctcccagcggcattcggttctcatcgaccgccacactggcgaggagttcacaccactgcagctgctggcgaccaactccgacggcagcccgaccggcggaaagttcctggtctacgactcgccactgtcggagtacgccgccgtcggcttcgagtacggctacactgtgggcaatccggacgccgtggtgctctgggaggcgcagttcggcgacttcgtcaacggcgcacagtcgatcatcgacgagttcatcagctccggtgaggccaagtggggccaattgtccaacgtcgtgctgctgttaccgcacgggcacgaggggcagggacccgaccacacttctgcccggatcgaacgcttcttgcagttgtgggcggaaggttcgatgaccatcgcgatgccgtcgactccgtcgaactacttccacctgctacgccggcatgccctggacggcatccaacgcccgctgatcgtgttcacgcccaagtcgatgttgcgtcacaaggccgccgtcagcgaaatcaaggacttcaccgagatcaagttccgctcagtgctggaggaacccacctatgaggacggcatcggagaccgcaacaaggtcagccggatcctgctgaccagtggcaagctgtattacgagctggccgcccgcaaggccaaggacaaccgcaatgacctcgcgatcgtgcggcttgaacagctcgccccgctgcccaggcgtcgactgcgtgaaacgctggaccgctacgagaacgtcaaggagttcttctgggtccaagaggaaccggccaaccagggtgcgtggccgcgattcgggctcgaactacccgagctgctgcctgacaagttggccgggatcaagcgaatctcgcgccgggcgatgtcagccccgtcgtcaggctcgtcgaaggtgcacgccgtcgaacagcaggagatcctcgacgaggcgttcggctaatctagcaagaggagaagtcgacatgaagttattaaaattggcacctgatgtttataaatttgatactgcagaggagtttatgaaatactttaaggttggaaaaggtgactttatacttactaatgaatttttatataaacctttccttgagaaattcaatgatggtgcagatgctgtatttcaggagaaatatggactcggtgaaccttctgatgaaatgataaacaatataattaaggatattggagataaacaatataatagaattattgctgtagggggaggatctgtaatagatatagccaaaatcctcagtcttaagtatactgatgattcattggatttgtttgagggaaaagtacctcttgtaaaaaacaaagaattaattatagttccaactacatgtggaacaggttcagaagttacaaatgtatcagttgcagaattaaagagaagacatactaaaaaaggaattgcttcagacgaattatatgcaacttatgcagtacttgtaccagaatttataaaaggacttccatataagttttttgtaaccagctccgtagatgccttaatacatgcaacagaagcttatgtatctccaaatgcaaatccttatactgatatgtttagtgtaaaagctatggagttaattttaaatggatacatgcaaatggtagagaaaggaaatgattacagagttgaaataattgaggattttgttataggcagcaattatgcaggtatagcttttggaaatgcaggagtgggagcggttcacgcactctcatatccaataggcggaaattatcatgtgcctcatggagaagcaaattatctgttttttacagaaatatttaaaacttattatgagaaaaatccaaatggcaagattaaagatgtaaataaactattagcaggcatactaaaatgtgatgaaagtgaagcttatgacagtttatcacaacttttagataaattattgtcaagaaaaccattaagagaatatggaatgaaagaggaagaaattgaaacttttgctgattcagtaatagaaggacagcagagactgttggtaaacaattatgaacctttttcaagagaagacatagtaaacacatataaaaagttatattaatctagaaagcttcctagaggcatcaaataaaacgaaaggctcagtcgaaagactgggcctttcgttttatctgttgtttgtcggtgaacgctctcctgagtaggacaaatccgccgccctagaccta

SEQ ID NO: 89 - DNA, 5097 bp, synthetic construct
  misc_feature 1..5097: note = Description of Artificial Sequence: Synthetic polynucleotide
  source 1..5097: mol_type = other DNA
tctgtatcaggctgaaaatcttctctcatccgccaaaacagcttcggcgttaagatgcgcgctcaaggacgtaagccgtcgactctcgccgtgctggcgcaggacacggctaccactcctttctctgttgatattctgcttgccattgagcaaaccgccagcgagttcggctggaatagttttttaatcaatattttttctgaagatgacgctgcccgcgcggcacgtcagctgcttgcccaccgtccggatggcattatctatactacaatggggctgcgacatatcacgctgcctgagtctctgtatggtgaaaatattgtattggcgaactgtgtggcggatgacccagcgttacccagttatatccctgatgattacactgcacaatatgaatcaacacagcatttgctcgcggcgggctatcgtcaaccgttatgcttctggctaccggaaagtgcgttggcaacagggtatcgtcggcagggatttgagcaggcctggcgtgatgctggacgagatctggctgaggtgaaacaatttcacatggcaacaggtgatgatcactacaccgatctcgcaagtttactcaatgcccacttcaaaccgggcaaaccagattttgatgttctgatatgtggtaacgatcgcgcagcctttgtggcttatcaggttcttctggcgaagggggtacgaatcccgcaggatgtcgccgtaatgggctttgataatctggttggcgtcgggcatctgtttttaccgccgctgaccacaattcagcttccacatgacattatcgggcgggaagctgcattgcatattattgaaggtcgtgaagggggaagagtgacgcggatcccttgcccgctgttgatccgttgttccacctgatattatgttaacccagtagccagagtgctccatgttgcagcacagccactccgtgggaggcataaagcgacagttcccgttcttctggctgcggatagattcgactactcatcaccgcttccccgtcgttaataaatacttccacggatgatgtatcgataaatatccttagggcgagcgtgtcacgctgcgggaggggaatactacggtagccgtctaaattctcgtgtgggtaataccgccacaaaacaagtcgctcagattggttatcaatatacagccgcattccagtgccgagctgtaatccgtaatgttcggcatcactgttcttcagcgcccactgcaactgaatctcaactgcttgcgcgttttcctgcaaaacatatttattgctgattgtgcggggagagacagattgatgctgctggcgtaacgactcagcttcgtgtaccgggcgttgtagaagtttgccattgctctctgatagctcgcgcgccagcgtcatgcagcctgcccatccttcacgttttgagggcattggcgattcccacatatccatccagccgataacaatacgccgaccatccttcgctaaaaagctttgtggtgcataaaagtcatgcccgttatcaagttcagtaaaatgcccggattgtgcaaaaagtcgtcctggcgaccacattccgggtattacgccactttgaaagcgatttcggtaactgtatccctcggcattcattccctgcggggaaaacatcagataatgctgatcgccaaggctgaaaaagtccggacattcccacatatagctttcacccgcatcagcgtgggccagtacgcgatcgaaggtccattcacgcaacgaactgccgcgataaagcaggatctgccccgtgttgcctggatctttcgccccgactaccatccaccatgtgtcggcttcacgccacactttaggatcgcggaagtgcatgattccttctggtggagtgaggatcacaccctgtttctcgaaatgaataccatcccgactggtagccagacattgtacttcgcgaattgcatcgtcattacctgcaccatcgagccagacgtgtccggtgtagataagtgagaggacaccattgtcatcgacagcactacctgaaaaacacccgtctttgtcattatcgtctcctggcgctagcgcaataggctcatgctgccagtggatcatatcgtcgctggtggcatgtccccagtgcattggcccccagtgttcgctcatcggatgatgttgataaaacgcgtgataacgatcgttaaaccagatcaggccgtttggatcgttcatccacccggcaggaggcgcgaggtgaaaatggggatagaaagtgttaccccggtgctcatgaagttttgctagggcgttttgcgccgcatgcaatcgagattgcgtcattttaatcatcctggttaagcaaatttggtgaattgttaacgttaacttttataaaaataaagtcccttactttcataaatgcgatgaatatcacaaatgttaacgttaactatgacgttttgtgatcgaatatgcatgttttagtaaatccatgacgattttgcgaaaaagaggtttatcactatgcgtaactcagatgaatttaagggaaaaaaatgtcagccaaagtatgggttttaggggatgcggtcgtagatctcttgccagaatcagacgggcgcctactgccttgtcctggcggcgcgccagctaacgttgcggtgggaatcgccagattaggcggaacaagtgggtttataggtcgggtgggggatgatccttttggtgcgttaatgcaaagaacgctgctaactgagggagtcgatatcacgtatctgaagcaagatgaatggcaccggacatccacggtgcttgtcgatctgaacgatcaaggggaacgttcatttacgtttatggtccgccccagtgccgatctttttttagagacgacagacttgccctgctggcgacatggcgaatggttacatctctgttcaattgcgttgtctgccgagccttcgcgtaccagcgcatttactgcgatgacggcgatccggcatgccggaggttttgtcagcttcgatcctaatattcgtgaagatctatggcaagacgagcatttgctccgcttgtgtttgcggcaggcgctacaactggcggatgtcgtcaagctctcggaagaagaatggcgacttatcagtggaaaaacacagaacgatcaggatatatgcgccctggcaaaagagtatgagatcgccatgctgttggtgactaaaggtgcagaaggggtggtggtctgttatcgaggacaagttcaccattttgctggaatgtctgtgaattgtgtcgatagcacgggggcgggagatgcgttcgttgccgggttactcacaggtctgtcctctacgggattatctacagatgagagagaaatgcgacgaattatcgatctcgctcaacgttgcggagcgcttgcagtaacggcgaaaggggcaatgacagcgctgccatgtcgacaagaactggaatagtgagaagtaaacggcgaagtcgctcttatctctaaataggacgtgaattttttaacgacaggcaggtaattatggcactgaatattccattcagaaatgcgtactatcgttttgcatccagttactcatttctcttttttatttcctggtcgctgtggtggtcgttatacgctatttggctgaaaggacatctagggttgacagggacggaattaggtacactttattcggtcaaccagtttaccagcattctatttatgatgttctacggcatcgttcaggataaactcggtctgaagaaaccgctcatctggtgtatgagtttcatcctggtcttgaccggaccgtttatgatttacgtttatgaaccgttactgcaaagcaatttttctgtaggtctaattctgggggcgctattttttggcttggggtatctggcgggatgcggtttgcttgatagcttcaccgaaaaaatggcgcgaaattttcatttcgaatatggaacagcgcgcgcctggggatcttttggctatgctattggcgcgttctttgccggcatattttttagtatcagtccccatatcaacttctggttggtctcgctatttggcgctgtatttatgatgatcaacatgcgttttaaagataaggatcaccagtgcgtagcggcagatgcgggaggggtaaaaaaagaggattttatcgcagttttcaaggatcgaaacttctgggttttcgtcatatttattgtggggacgtggtctttctataacatttttgatcaacaactttttcctgtcttttattcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggagttgtgattatggcgttgcgtatcctttcctgcgcgctgttcgttaacccctggattatttcattagtgaagttgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcttgagtaaaaaacgcgagcaaatagttatggaaacgcctgtaccttcagcaatatagacgtaaactttttccggttgttgtcgatagctctatatccctcaaccggaaaataataatagtaaaatgcttagccctgctaataatcgcctaatccaaacgcctcattcatgttctggtacagtcgctcaaatgtacttcagatgcgcggttcgctgatttccaggacattgtcgtcattcagtgacctgtcccgtgtatcacggtcctgcgaattcatcaaggaatgcattgcggagtgaagtatcgagtcacgccatatttcgtcacccgaagatgagttttgagatattaaggcaggtgactttcactcaca

SEQ ID NO: 90 - DNA, 3525 bp, Nocardia iowensis
  source 1..3525: mol_type = other DNA
atggcagtggattcaccggatgagcggctacagcgccgcattgcacagttgtttgcagaagatgagcaggtcaaggccgcacgtccgctcgaagcggtgagcgcggcggtgagcgcgcccggtatgcggctggcgcagatcgccgccactgttatggcgggttacgccgaccgcccggccgccgggcagcgtgcgttcgaactgaacaccgacgacgcgacgggccgcacctcgctgcggttacttccccgattcgagaccatcacctatcgcgaactgtggcagcgagtcggcgaggttgccgcggcctggcatcatgatcccgagaaccccttgcgcgcaggtgatttcgtcgccctgctcggcttcaccagcatcgactacgccaccctcgacctggccgatatccacctcggcgcggttaccgtgccgttgcaggccagcgcggcggtgtcccagctgatcgctatcctcaccgagacttcgccgcggctgctcgcctcgaccccggagcacctcgatgcggcggtcgagtgcctactcgcgggcaccacaccggaacgactggtggtcttcgactaccaccccgaggacgacgaccagcgtgcggccttcgaatccgcccgccgccgccttgccgacgcgggcagcttggtgatcgtcgaaacgctcgatgccgtgcgtgcccggggccgcgacttaccggccgcgccactgttcgttcccgacaccgacgacgacccgctggccctgctgatctacacctccggcagcaccggaacgccgaagggcgcgatgtacaccaatcggttggccgccacgatgtggcaggggaactcgatgctgcaggggaactcgcaacgggtcgggatcaatctcaactacatgccgatgagccacatcgccggtcgcatatcgctgttcggcgtgctcgctcgcggtggcaccgcatacttcgcggccaagagcgacatgtcgacactgttcgaagacatcggcttggtacgtcccaccgagatcttcttcgtcccgcgcgtgtgcgacatggtcttccagcgctatcagagcgagctggaccggcgctcggtggcgggcgccgacctggacacgctcgatcgggaagtgaaagccgacctccggcagaactacctcggtgggcgcttcctggtggcggtcgtcggcagcgcgccgctggccgcggagatgaagacgttcatggagtccgtcctcgatctgccactgcacgacgggtacgggtcgaccgaggcgggcgcaagcgtgctgctcgacaaccagatccagcggccgccggtgctcgattacaagctcgtcgacgtgcccgaactgggttacttccgcaccgaccggccgcatccgcgcggtgagctgttgttgaaggcggagaccacgattccgggctactacaagcggcccgaggtcaccgcggagatcttcgacgaggacggcttctacaagaccggcgatatcgtggccgagctcgagcacgatcggctggtctatgtcgaccgtcgcaacaatgtgctcaaactgtcgcagggcgagttcgtgaccgtcgcccatctcgaggccgtgttcgccagcagcccgctgatccggcagatcttcatctacggcagcagcgaacgttcctatctgctcgcggtgatcgtccccaccgacgacgcgctgcgcggccgcgacaccgccaccttgaaatcggcactggccgaatcgattcagcgcatcgccaaggacgcgaacctgcagccctacgagattccgcgcgatttcctgatcgagaccgagccgttcaccatcgccaacggactgctctccggcatcgcgaagctgctgcgccccaatctgaaggaacgctacggcgctcagctggagcagatgtacaccgatctcgcgacaggccaggccgatgagctgctcgccctgcgccgcgaagccgccgacctgccggtgctcgaaaccgtcagccgggcagcgaaagcgatgctcggcgtcgcctccgccgatatgcgtcccgacgcgcacttcaccgacctgggcggcgattccctttccgcgctgtcgttctcgaacctgctgcacgagatcttcggggtcgaggtgccggtgggtgtcgtcgtcagcccggcgaacgagctgcgcgatctggcgaattacattgaggcggaacgcaactcgggcgcgaagcgtcccaccttcacctcggtgcacggcggcggttccgagatccgcgccgccgatctgaccctcgacaagttcatcgatgcccgcaccctggccgccgccgacagcattccgcacgcgccggtgccagcgcagacggtgctgctgaccggcgcgaacggctacctcggccggttcctgtgcctggaatggctggagcggctggacaagacgggtggcacgctgatctgcgtcgtgcgcggtagtgacgcggccgcggcccgtaaacggctggactcggcgttcgacagcggcgatcccggcctgctcgagcactaccagcaactggccgcacggaccctggaagtcctcgccggtgatatcggcgacccgaatctcggtctggacgacgcgacttggcagcggttggccgaaaccgtcgacctgatcgtccatcccgccgcgttggtcaaccacgtccttccctacacccagctgttcggccccaatgtcgtcggcaccgccgaaatcgtccggttggcgatcacggcgcggcgcaagccggtcacctacctgtcgaccgtcggagtggccgaccaggtcgacccggcggagtatcaggaggacagcgacgtccgcgagatgagcgcggtgcgcgtcgtgcgcgagagttacgccaacggctacggcaacagcaagtgggcgggggaggtcctgctgcgcgaagcacacgatctgtgtggcttgccggtcgcggtgttccgttcggacatgatcctggcgcacagccggtacgcgggtcagctcaacgtccaggacgtgttcacccggctgatcctcagcctggtcgccaccggcatcgcgccgtactcgttctaccgaaccgacgcggacggcaaccggcagcgggcccactatgacggcttgccggcggacttcacggcggcggcgatcaccgcgctcggcatccaagccaccgaaggcttccggacctacgacgtgctcaatccgtacgacgatggcatctccctcgatgaattcgtcgactggctcgtcgaatccggccacccgatccagcgcatcaccgactacagcgactggttccaccgtttcgagacggcgatccgcgcgctgccggaaaagcaacgccaggcctcggtgctgccgttgctggacgcctaccgcaacccctgcccggcggtccgcggcgcgatactcccggccaaggagttccaagcggcggtgcaaacagccaaaatcggtccggaacaggacatcccgcatttgtccgcgccactgatcgataagtacgtcagcgatctggaactgcttcagctgctctaa

SEQ ID NO: 91 - protein, 1174 aa, Nocardia iowensis
MAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSLVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 92 - DNA, 669 bp, synthetic construct
  misc_feature 1..669: note = Description of Artificial Sequence: Synthetic polynucleotide
  source 1..669: mol_type = other DNA
atgattgaaaccattctgcctgcaggcgttgaaagcgcagaactgctggaatatccggaagatctgaaagcacatccggcagaagaacatctgattgccaaaagcgttgaaaaacgtcgtcgtgattttattggtgcacgtcattgtgcacgtctggcactggcagaactgggtgaacctccggttgcaattggtaaaggtgaacgtggtgcaccgatttggcctcgtggtgttgttggtagcctgacccattgtgatggttatcgtgcagcagcagttgcacataaaatgcgctttcgcagcattggtattgatgcagaaccgcatgcaaccctgccggaaggtgttctggatagcgttagcctgccgccggaacgtgaatggctgaaaaccaccgatagcgcactgcatctggatcgtctgctgttttgtgcaaaagaagccacctataaagcctggtggccgctgacagcacgttggctgggttttgaagaagcccatattacctttgaaattgaagatggtagcgcagatagcggtaatggcacctttcatagcgaactgctggttccgggtcagaccaatgatggtggtacaccgctgctgagctttgatggtcgttggctgattgcagatggttttattctgaccgcaattgcctatgcctaa

SEQ ID NO: 93 - protein, 222 aa, synthetic construct
  REGION 1..222: note = Description of Artificial Sequence: Synthetic polypeptide
MIETILPAGVESAELLEYPEDLKAHPAEEHLIAKSVEKRRRDFIGARHCARLALAELGEPPVAIGKGERGAPIWPRGVVGSLTHCDGYRAAAVAHKMRFRSIGIDAEPHATLPEGVLDSVSLPPEREWLKTTDSALHLDRLLFCAKEATYKAWWPLTARWLGFEEAHITFEIEDGSADSGNGTFHSELLVPGQTNDGGTPLLSFDGRWLIADGFILTAIAYA

SEQ ID NO: 94 - DNA, 3522 bp, Mycobacterium smegmatis
  source 1..3522: mol_type = other DNA
atgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctaa

SEQ ID NO: 95 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 96 - DNA, 3522 bp, Mycobacterium avium
  source 1..3522: mol_type = other DNA
atgtcgactgccacccatgacgaacgactcgaccgtcgcgtccacgaactcatcgccaccgacccgcaattcgccgccgcccaacccgacccggcgatcaccgccgccctcgaacagcccgggctgcggctgccgcagatcatccgcaccgtgctcgacggctacgccgaccggccggcgctgggacagcgcgtggtggagttcgtcacggacgccaagaccgggcgcacgtcggcgcagctgctcccccgcttcgagaccatcacgtacagcgaagtagcgcagcgtgtttcggcgctgggccgcgccctgtccgacgacgcggtgcaccccggcgaccgggtgtgcgtgctgggcttcaacagcgtcgactacgccaccatcgacatggcgctgggcgccatcggcgccgtctcggtgccgctgcagaccagcgcggcaatcagctcgctgcagccgatcgtggccgagaccgagcccaccctgatcgcgtccagcgtgaaccagctgtccgacgcggtgcagctgatcaccggcgccgagcaggcgcccacccggctggtggtgttcgactaccacccgcaggtcgacgaccagcgcgaggccgtccaggacgccgcggcgcggctgtccagcaccggcgtggccgtccagacgctggccgagctgctggagcgcggcaaggacctgcccgccgtcgcggagccgcccgccgacgaggactcgctggccctgctgatctacacctccgggtccaccggcgcccccaagggcgcgatgtacccacagagcaacgtcggcaagatgtggcgccgcggcagcaagaactggttcggcgagagcgccgcgtcgatcaccctgaacttcatgccgatgagccacgtgatgggccgaagcatcctctacggcacgctgggcaacggcggcaccgcctacttcgccgcccgcagcgacctgtccaccctgcttgaggacctcgagctggtgcggcccaccgagctcaacttcgtcccgcggatctgggagacgctgtacggcgaattccagcgtcaggtcgagcggcggctctccgaggccggggacgccggcgaacgtcgcgccgtcgaggccgaggtgctggccgagcagcgccagtacctgctgggcgggcggttcaccttcgcgatgacgggctcggcgcccatctcgccggagctgcgcaactgggtcgagtcgctgctcgaaatgcacctgatggacggctacggctccaccgaggccggaatggtgttgttcgacggggagattcagcgcccgccggtgatcgactacaagctggtcgacgtgccggacctgggctacttcagcaccgaccggccgcatccgcgcggcgagctgctgctgcgcaccgagaacatgttcccgggctactacaagcgggccgaaaccaccgcgggcgtcttcgacgaggacggctactaccgcaccggcgacgtgttcgccgagatcgccccggaccggctggtctacgtcgaccgccgcaacaacgtgctcaagctggcgcagggcgaattcgtcacgctggccaagctggaggcggtgttcggcaacagcccgctgatccgccagatctacgtctacggcaacagcgcccagccctacctgctggcggtcgtggtgcccaccgaggaggcgctggcctcgggtgaccccgagacgctcaagcccaagatcgccgactcgctgcagcaggtcgccaaggaggccggcctgcagtcctacgaggtgccgcgcgacttcatcatcgagaccaccccgttcagcctggaaaacggtctgctgaccgggatccggaagctggcgtggccgaaactgaagcagcactacggggaacggctggagcagatgtacgccgacctggccgccggacaggccaacgagctggccgagctgcgccgcaacggtgcccaggcgccggtgttgcagaccgtgagccgcgccgcgggcgccatgctgggttcggccgcctccgacctgtcccccgacgcccacttcaccgatctgggcggagactcgttgtcggcgttgacattcggcaacctgctgcgcgagatcttcgacgtcgacgtgccggtaggcgtgatcgtcagcccggccaacgacctggcggccatcgcgagctacatcgaggccgagcggcagggcagcaagcgcccgacgttcgcctcggtgcacggccgggacgcgaccgtggtgcgcgccgccgacctgacgctggacaagttcctcgacgccgagacgctggccgccgcgccgaacctgcccaagccggccaccgaggtgcgcaccgtgctgctgaccggcgccaccggcttcctgggccgctacctggccctggaatggctggagcggatggacatggtggacggcaaggtcatcgccctggtccgggcccgctccgacgaggaggcacgcgcccggctggacaagaccttcgacagcggcgacccgaaactgctcgcgcactaccagcagctggccgccgatcacctggaggtcatcgccggcgacaagggcgaggccaatctgggcctgggccaagacgtttggcaacgactggccgacacggtcgacgtgatcgtcgaccccgccgcgctggtcaaccacgtgttgccgtacagcgagctgttcgggcccaacgccctgggcaccgcggagctgatccggctggcgctgacgtccaagcagaagccgtacacctacgtgtccaccatcggcgtgggcgaccagatcgagccgggcaagttcgtcgagaacgccgacatccggcagatgagcgccacccgggcgatcaacgacagctacgccaacggctatggcaacagcaagtgggccggcgaggtgctgctgcgcgaggcgcacgacctgtgcgggctgcccgtcgcggtgttccgctgcgacatgatcctggccgacaccacgtatgccgggcagctcaacctgccggacatgttcacccggctgatgctgagcctggtggccaccgggatcgcgcccggctcgttctacgagctcgacgccgacggcaaccggcagcgggcgcactacgacggcctgccggtcgagttcatcgccgcggcgatctcgacgctgggttcgcagatcaccgacagcgacaccggcttccagacctaccacgtgatgaacccctacgatgacggcgtcggtctggacgagtacgtcgattggctggtggacgccggctattcgatcgagcggatcgccgactactccgaatggctgcggcggttcgagacctcgctgcgggccctgccggaccggcagcgccagtactcgctgctgccgctgctgcacaactaccgcacgccggagaagccgatcaacgggtcgatagctcccaccgacgtgttccgggcagcggtgcaggaggcgaaaatcggccccgacaaagacattccgcacgtgtcgccgccggtcatcgtcaagtacatcaccgacctgcagctgctcgggctgctctaa

SEQ ID NO: 97 - protein, 1173 aa, Mycobacterium avium
MSTATHDERLDRRVHELIATDPQFAAAQPDPAITAALEQPGLRLPQIIRTVLDGYADRPALGQRVVEFVTDAKTGRTSAQLLPRFETITYSEVAQRVSALGRALSDDAVHPGDRVCVLGFNSVDYATIDMALGAIGAVSVPLQTSAAISSLQPIVAETEPTLIASSVNQLSDAVQLITGAEQAPTRLVVFDYHPQVDDQREAVQDAAARLSSTGVAVQTLAELLERGKDLPAVAEPPADEDSLALLIYTSGSTGAPKGAMYPQSNVGKMWRRGSKNWFGESAASITLNFMPMSHVMGRSILYGTLGNGGTAYFAARSDLSTLLEDLELVRPTELNFVPRIWETLYGEFQRQVERRLSEAGDAGERRAVEAEVLAEQRQYLLGGRFTFAMTGSAPISPELRNWVESLLEMHLMDGYGSTEAGMVLFDGEIQRPPVIDYKLVDVPDLGYFSTDRPHPRGELLLRTENMFPGYYKRAETTAGVFDEDGYYRTGDVFAEIAPDRLVYVDRRNNVLKLAQGEFVTLAKLEAVFGNSPLIRQIYVYGNSAQPYLLAVVVPTEEALASGDPETLKPKIADSLQQVAKEAGLQSYEVPRDFIIETTPFSLENGLLTGIRKLAWPKLKQHYGERLEQMYADLAAGQANELAELRRNGAQAPVLQTVSRAAGAMLGSAASDLSPDAHFTDLGGDSLSALTFGNLLREIFDVDVPVGVIVSPANDLAAIASYIEAERQGSKRPTFASVHGRDATVVRAADLTLDKFLDAETLAAAPNLPKPATEVRTVLLTGATGFLGRYLALEWLERMDMVDGKVIALVRARSDEEARARLDKTFDSGDPKLLAHYQQLAADHLEVIAGDKGEANLGLGQDVWQRLADTVDVIVDPAALVNHVLPYSELFGPNALGTAELIRLALTSKQKPYTYVSTIGVGDQIEPGKFVENADIRQMSATRAINDSYANGYGNSKWAGEVLLREAHDLCGLPVAVFRCDMILADTTYAGQLNLPDMFTRLMLSLVATGIAPGSFYELDADGNRQRAHYDGLPVEFIAAAISTLGSQITDSDTGFQTYHVMNPYDDGVGLDEYVDWLVDAGYSIERIADYSEWLRRFETSLRALPDRQRQYSLLPLLHNYRTPEKPINGSIAPTDVFRAAVQEAKIGPDKDIPHVSPPVIVKYITDLQLLGLL

SEQ ID NO: 98 - DNA, 3525 bp, Mycobacterium marinum
  source 1..3525: mol_type = other DNA
atgtcgccaatcacgcgtgaagagcggctcgagcgccgcatccaggacctctacgccaacgacccgcagttcgccgccgccaaacccgccacggcgatcaccgcagcaatcgagcggccgggtctaccgctaccccagatcatcgagaccgtcatgaccggatacgccgatcggccggctctcgctcagcgctcggtcgaattcgtgaccgacgccggcaccggccacaccacgctgcgactgctcccccacttcgaaaccatcagctacggcgagctttgggaccgcatcagcgcactggccgacgtgctcagcaccgaacagacggtgaaaccgggcgaccgggtctgcttgttgggcttcaacagcgtcgactacgccacgatcgacatgactttggcgcggctgggcgcggtggccgtaccactgcagaccagcgcggcgataacccagctgcagccgatcgtcgccgagacccagcccaccatgatcgcggccagcgtcgacgcactcgctgacgccaccgaattggctctgtccggtcagaccgctacccgagtcctggtgttcgaccaccaccggcaggttgacgcacaccgcgcagcggtcgaatccgcccgggagcgcctggccggctcggcggtcgtcgaaaccctggccgaggccatcgcgcgcggcgacgtgccccgcggtgcgtccgccggctcggcgcccggcaccgatgtgtccgacgactcgctcgcgctactgatctacacctcgggcagcacgggtgcgcccaagggcgcgatgtacccccgacgcaacgttgcgaccttctggcgcaagcgcacctggttcgaaggcggctacgagccgtcgatcacgctgaacttcatgccaatgagccacgtcatgggccgccaaatcctgtacggcacgctgtgcaatggcggcaccgcctacttcgtggcgaaaagcgatctctccaccttgttcgaagacctggcgctggtgcggcccaccgagctgaccttcgtgccgcgcgtgtgggacatggtgttcgacgagtttcagagtgaggtcgaccgccgcctggtcgacggcgccgaccgggtcgcgctcgaagcccaggtcaaggccgagatacgcaacgacgtgctcggtggacggtataccagcgcactgaccggctccgcccctatctccgacgagatgaaggcgtgggtcgaggagctgctcgacatgcatctggtcgagggctacggctccaccgaggccgggatgatcctgatcgacggagccattcggcgcccggcggtactcgactacaagctggtcgatgttcccgacctgggttacttcctgaccgaccggccacatccgcggggcgagttgctggtcaagaccgatagtttgttcccgggctactaccagcgagccgaagtcaccgccgacgtgttcgatgctgacggcttctaccggaccggcgacatcatggccgaggtcggccccgaacagttcgtgtacctcgaccgccgcaacaacgtgttgaagctgtcgcagggcgagttcgtcaccgtctccaaactcgaagcggtgtttggcgacagcccactggtacggcagatctacatctacggcaacagcgcccgtgcctacctgttggcggtgatcgtccccacccaggaggcgctggacgccgtgcctgtcgaggagctcaaggcgcggctgggcgactcgctgcaagaggtcgcaaaggccgccggcctgcagtcctacgagatcccgcgcgacttcatcatcgaaacaacaccatggacgctggagaacggcctgctcaccggcatccgcaagttggccaggccgcagctgaaaaagcattacggcgagcttctcgagcagatctacacggacctggcacacggccaggccgacgaactgcgctcgctgcgccaaagcggtgccgatgcgccggtgctggtgacggtgtgccgtgcggcggccgcgctgttgggcggcagcgcctctgacgtccagcccgatgcgcacttcaccgatttgggcggcgactcgctgtcggcgctgtcgttcaccaacctgctgcacgagatcttcgacatcgaagtgccggtgggcgtcatcgtcagccccgccaacgacttgcaggccctggccgactacgtcgaggcggctcgcaaacccggctcgtcacggccgaccttcgcctcggtccacggcgcctcgaatgggcaggtcaccgaggtgcatgccggtgacctgtccctggacaaattcatcgatgccgcaaccctggccgaagctccccggctgcccgccgcaaacacccaagtgcgcaccgtgctgctgaccggcgccaccggcttcctcgggcgctacctggccctggaatggctggagcggatggacctggtcgacggcaaactgatctgcctggtccgggccaagtccgacaccgaagcacgggcgcggctggacaagacgttcgacagcggcgaccccgaactgctggcccactaccgcgcactggccggcgaccacctcgaggtgctcgccggtgacaagggcgaagccgacctcggactggaccggcagacctggcaacgcctggccgacacggtcgacctgatcgtcgaccccgcggccctggtcaaccacgtactgccatacagccagctgttcgggcccaacgcgctgggcaccgccgagctgctgcggctggcgctcacctccaagatcaagccctacagctacacctcgacaatcggtgtcgccgaccagatcccgccgtcggcgttcaccgaggacgccgacatccgggtcatcagcgccacccgcgcggtcgacgacagctacgccaatggctactcgaacagcaagtgggccggcgaggtgctgttgcgcgaggcgcatgacctgtgtggcctgccggttgcggtgttccgctgcgacatgatcctggccgacaccacatgggcgggacagctcaatgtgccggacatgttcacccggatgatcctgagcctggcggccaccggtatcgcgccgggttcgttctatgagcttgcggccgacggcgcccggcaacgcgcccactatgacggtctgcccgtcgagttcatcgccgaggcgatttcgactttgggtgcgcagagccaggatggtttccacacgtatcacgtgatgaacccctacgacgacggcatcggactcgacgagttcgtcgactggctcaacgagtccggttgccccatccagcgcatcgctgactatggcgactggctgcagcgcttcgaaaccgcactgcgcgcactgcccgatcggcagcggcacagctcactgctgccgctgttgcacaactatcggcagccggagcggcccgtccgcgggtcgatcgcccctaccgatcgcttccgggcagcggtgcaagaggccaagatcggccccgacaaagacattccgcacgtcggcgcgccgatcatcgtgaagtacgtcagcgacctgcgcctactcggcctgctctaa

SEQ ID NO: 99 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 100 - DNA, 40 bp, synthetic construct
  misc_feature 1..40: note = Description of Artificial Sequence: Synthetic primer
  source 1..40: mol_type = other DNA
gaggagaagtcgacatgaacgaacaatattccgcattgcg

SEQ ID NO: 101 - DNA, 39 bp, synthetic construct
  misc_feature 1..39: note = Description of Artificial Sequence: Synthetic primer
  source 1..39: mol_type = other DNA
ggaagctttctagattagccggtattacgcatacctgcc

SEQ ID NO: 102 - DNA, 47 bp, synthetic construct
  misc_feature 1..47: note = Description of Artificial Sequence: Synthetic primer
  source 1..47: mol_type = other DNA
taagctagcaagaggagaagtcgacatgcagaacagcgctttgaaag

SEQ ID NO: 103 - DNA, 43 bp, synthetic construct
  misc_feature 1..43: note = Description of Artificial Sequence: Synthetic primer
  source 1..43: mol_type = other DNA
gcctctaggaagctttctagattactttttcttcgctttggcg

SEQ ID NO: 104 - DNA, 49 bp, synthetic construct
  misc_feature 1..49: note = Description of Artificial Sequence: Synthetic primer
  source 1..49: mol_type = other DNA
ctagcaagaggagaagtcgacatgcctgacgctaaaaaacaggggcggt

SEQ ID NO: 105 - DNA, 48 bp, synthetic construct
  misc_feature 1..48: note = Description of Artificial Sequence: Synthetic primer
  source 1..48: mol_type = other DNA
ctaggaagctttctagagtcgttaatcgtgagcgcctatttcgcgcag

SEQ ID NO: 106 - DNA, 51 bp, synthetic construct
  misc_feature 1..51: note = Description of Artificial Sequence: Synthetic primer
  source 1..51: mol_type = other DNA
ctagcaagaggagaagtcgacatgacaaagtatgcattagtcggtgatgtg

SEQ ID NO: 107 - DNA, 54 bp, synthetic construct
  misc_feature 1..54: note = Description of Artificial Sequence: Synthetic primer
  source 1..54: mol_type = other DNA
ctaggaagctttctagagtcgttacagaatgtgacctaaggtctggcgtaaatg

SEQ ID NO: 108 - DNA, 51 bp, synthetic construct
  misc_feature 1..51: note = Description of Artificial Sequence: Synthetic primer
  source 1..51: mol_type = other DNA
ctagcaagaggagaagtcgacatgagttctgaaagtagtcagggtctagtc

SEQ ID NO: 109 - DNA, 45 bp, synthetic construct
  misc_feature 1..45: note = Description of Artificial Sequence: Synthetic primer
  source 1..45: mol_type = other DNA
ctaggaagctttctagagtcgttacttctgggagcgccacatctc

SEQ ID NO: 110 - DNA, 45 bp, synthetic construct
  misc_feature 1..45: note = Description of Artificial Sequence: Synthetic primer
  source 1..45: mol_type = other DNA
tagcaagaggagaagtcgacatggactggaagaagatctatgaag

SEQ ID NO: 111 - DNA, 39 bp, synthetic construct
  misc_feature 1..39: note = Description of Artificial Sequence: Synthetic primer
  source 1..39: mol_type = other DNA
cctctaggaagctttctagattagaatgccgcgttgaag

SEQ ID NO: 112 - DNA, 42 bp, synthetic construct
  misc_feature 1..42: note = Description of Artificial Sequence: Synthetic primer
  source 1..42: mol_type = other DNA
tagcaagaggagaagtcgacatgagctggcaagaactgtatc

SEQ ID NO: 113 - DNA, 47 bp, synthetic construct
  misc_feature 1..47: note = Description of Artificial Sequence: Synthetic primer
  source 1..47: mol_type = other DNA
cctctaggaagctttctagattaatatttctctttaaagcgcttttc

SEQ ID NO: 114 - DNA, 43 bp, synthetic construct
  misc_feature 1..43: note = Description of Artificial Sequence: Synthetic primer
  source 1..43: mol_type = other DNA
tagcaagaggagaagtcgacatggcacgttttactttaccaag

SEQ ID NO: 115 - DNA, 52 bp, synthetic construct
  misc_feature 1..52: note = Description of Artificial Sequence: Synthetic primer
  source 1..52: mol_type = other DNA
cctctaggaagctttctagattacaaattaactttagttccatagtatgtgc

SEQ ID NO: 116 - DNA, 100 bp, synthetic construct
  misc_feature 1..100: note = Description of Artificial Sequence: Synthetic primer
  source 1..100: mol_type = other DNA
accggtaaacgactcagcagcctgacgcactggcgatattattttgccttctcctcaccacagaatgttctgccacctgacgatatcaaattacgccccg

SEQ ID NO: 117 - DNA, 104 bp, synthetic construct
  misc_feature 1..104: note = Description of Artificial Sequence: Synthetic primer
  source 1..104: mol_type = other DNA
tgcttatccacaacattttgcgcacggttatgtggacaaaatacctggttacccaggccgtgccggcacgttaaccgggccctaggtctagggcggcggatttg

SEQ ID NO: 118 - DNA, 20 bp, synthetic construct
  misc_feature 1..20: note = Description of Artificial Sequence: Synthetic primer
  source 1..20: mol_type = other DNA
aggcagttccataggatggc

SEQ ID NO: 119 - DNA, 40 bp, synthetic construct
  misc_feature 1..40: note = Description of Artificial Sequence: Synthetic primer
  source 1..40: mol_type = other DNA
ataataataggtaccggcggcgctggcgcagcttgctgcg

SEQ ID NO: 120 - DNA, 41 bp, synthetic construct
  misc_feature 1..41: note = Description of Artificial Sequence: Synthetic primer
  source 1..41: mol_type = other DNA
tattattatggatccaacccgataatggtagatctccctct

SEQ ID NO: 121 - DNA, 41 bp, synthetic construct
  misc_feature 1..41: note = Description of Artificial Sequence: Synthetic primer
  source 1..41: mol_type = other DNA
ataataataggatccggagcagaaacaatgtggtatttact

SEQ ID NO: 122 - DNA, 43 bp, synthetic construct
  misc_feature 1..43: note = Description of Artificial Sequence: Synthetic primer
  source 1..43: mol_type = other DNA
tattattatctgcagatgctcttttttatgcattacaaactgc

SEQ ID NO: 123 - DNA, 35 bp, synthetic construct
  misc_feature 1..35: note = Description of Artificial Sequence: Synthetic primer
  source 1..35: mol_type = other DNA
taataataaggtaccaactgggcttgcttcactgg

SEQ ID NO: 124 - DNA, 35 bp, synthetic construct
  misc_feature 1..35: note = Description of Artificial Sequence: Synthetic primer
  source 1..35: mol_type = other DNA
taataataaagatctgcctcgaccgttcaggaagg

SEQ ID NO: 125 - DNA, 38 bp, synthetic construct
  misc_feature 1..38: note = Description of Artificial Sequence: Synthetic primer
  source 1..38: mol_type = other DNA
taataataaagatcttaactctccagtaacaaagctgc

SEQ ID NO: 126 - DNA, 34 bp, synthetic construct
  misc_feature 1..34: note = Description of Artificial Sequence: Synthetic primer
  source 1..34: mol_type = other DNA
taataataactgcaggctatgtcaccacttacgg

SEQ ID NO: 127 - DNA, 37 bp, synthetic construct
  misc_feature 1..37: note = Description of Artificial Sequence: Synthetic primer
  source 1..37: mol_type = other DNA
tattattattcctgcaggcgtctttgttcttccgtcc

SEQ ID NO: 128 - DNA, 38 bp, synthetic construct
  misc_feature 1..38: note = Description of Artificial Sequence: Synthetic primer
  source 1..38: mol_type = other DNA
tattattatcttcggtacctttagcatctgcttcggcc

SEQ ID NO: 129 - DNA, 39 bp, synthetic construct
  misc_feature 1..39: note = Description of Artificial Sequence: Synthetic primer
  source 1..39: mol_type = other DNA
tattattatggatcccggtaatgcaacaaaagttagagc

SEQ ID NO: 130 - DNA, 48 bp, synthetic construct
  misc_feature 1..48: note = Description of Artificial Sequence: Synthetic primer
  source 1..48: mol_type = other DNA
tattattatggatccaaagtgactgacatgaatatccttgatttgttc

SEQ ID NO: 131 - DNA, 39 bp, synthetic construct
  misc_feature 1..39: note = Description of Artificial Sequence: Synthetic primer
  source 1..39: mol_type = other DNA
taataataaggatcctacactggcggatgtggcataaac

SEQ ID NO: 132 - DNA, 39 bp, synthetic construct
  misc_feature 1..39: note = Description of Artificial Sequence: Synthetic primer
  source 1..39: mol_type = other DNA
taataatttggtacccattcacagtcaccaggtacaacg

SEQ ID NO: 133 - DNA, 39 bp, synthetic construct
  misc_feature 1..39: note = Description of Artificial Sequence: Synthetic primer
  source 1..39: mol_type = other DNA
taataataaggatccgagaataacatgaatggtgcattg

SEQ ID NO: 134 - DNA, 39 bp, synthetic construct
  misc_feature 1..39: note = Description of Artificial Sequence: Synthetic primer
  source 1..39: mol_type = other DNA
taataatatctgcagatccacaaaaagccctggcaattg

SEQ ID NO: 135 - DNA, 31 bp, synthetic construct
  misc_feature 1..31: note = Description of Artificial Sequence: Synthetic primer
  source 1..31: mol_type = other DNA
gataatcccccctgcagggagcggtaaatag

SEQ ID NO: 136 - DNA, 34 bp, synthetic construct
  misc_feature 1..34: note = Description of Artificial Sequence: Synthetic primer
  source 1..34: mol_type = other DNA
tattattttggatccttctctggtgttgtttggg

SEQ ID NO: 137 - DNA, 33 bp, synthetic construct
  misc_feature 1..33: note = Description of Artificial Sequence: Synthetic primer
  source 1..33: mol_type = other DNA
tattattatggatccgcgtggtgttgaaagccg

SEQ ID NO: 138 - DNA, 38 bp, synthetic construct
  misc_feature 1..38: note = Description of Artificial Sequence: Synthetic primer
  source 1..38: mol_type = other DNA
tattataaaggtaccgcctcttccaggtcagtgaaggg

SEQ ID NO: 139 - DNA, 36 bp, synthetic construct
  misc_feature 1..36: note = Description of Artificial Sequence: Synthetic primer
  source 1..36: mol_type = other DNA
taataataaggtacccagttttggctatgccttaag

SEQ ID NO: 140 - DNA, 34 bp, synthetic construct
  misc_feature 1..34: note = Description of Artificial Sequence: Synthetic primer
  source 1..34: mol_type = other DNA
taataatcattaagcttgctccctttgctgggcc

SEQ ID NO: 141 - DNA, 35 bp, synthetic construct
  misc_feature 1..35: note = Description of Artificial Sequence: Synthetic primer
  source 1..35: mol_type = other DNA
taataataataagctttttacgcctcaaactttcg

SEQ ID NO: 142 - DNA, 41 bp, synthetic construct
  misc_feature 1..41: note = Description of Artificial Sequence: Synthetic primer
  source 1..41: mol_type = other DNA
taataataaattgccctgcagcgtaagattgtcgttcaggg

SEQ ID NO: 143 - DNA, 31 bp, synthetic construct
  misc_feature 1..31: note = Description of Artificial Sequence: Synthetic primer
  source 1..31: mol_type = other DNA
tattattattgtttggtacctctgtgccgct

SEQ ID NO: 144 - DNA, 33 bp, synthetic construct
  misc_feature 1..33: note = Description of Artificial Sequence: Synthetic primer
  source 1..33: mol_type = other DNA
aatgttccactctgcagggatgataataagggg

SEQ ID NO: 145 - DNA, 29 bp, synthetic construct
  misc_feature 1..29: note = Description of Artificial Sequence: Synthetic primer
  source 1..29: mol_type = other DNA
atcggatccaatcgcacactaacagactg

SEQ ID NO: 146 - DNA, 38 bp, synthetic construct
  misc_feature 1..38: note = Description of Artificial Sequence: Synthetic primer
  source 1..38: mol_type = other DNA
acagctttggatcctcattgtgtttactcctgattagc

SEQ ID NO: 147 - DNA, 42 bp, synthetic construct
  misc_feature 1..42: note = Description of Artificial Sequence: Synthetic primer
  source 1..42: mol_type = other DNA
ataataatatctagagcagcaagccgcgcggcaggtggtcag

SEQ ID NO: 148 - DNA, 42 bp, synthetic construct
  misc_feature 1..42: note = Description of Artificial Sequence: Synthetic primer
  source 1..42: mol_type = other DNA
tattattatctgcagacgccatcctgatccatatgtatatgg

SEQ ID NO: 149 - DNA, 42 bp, synthetic construct
  misc_feature 1..42: note = Description of Artificial Sequence: Synthetic primer
  source 1..42: mol_type = other DNA
tattattatggatccagttcctccttttcggatgatgttctg

SEQ ID NO: 150 - DNA, 42 bp, synthetic construct
  misc_feature 1..42: note = Description of Artificial Sequence: Synthetic primer
  source 1..42: mol_type = other DNA
ataataataggatccgaggcctttgctgcgactgccatgttc

SEQ ID NO: 151 - DNA, 20 bp, synthetic construct
  misc_feature 1..20: note = Description of Artificial Sequence: Synthetic primer
  source 1..20: mol_type = other DNA
cgccacaaccagtgacaccc

SEQ ID NO: 152 - DNA, 24 bp, synthetic construct
  misc_feature 1..24: note = Description of Artificial Sequence: Synthetic primer
  source 1..24: mol_type = other DNA
ggggttttagtcgccctttctggc

SEQ ID NO: 153 - DNA, 24 bp, synthetic construct
  misc_feature 1..24: note = Description of Artificial Sequence: Synthetic primer
  source 1..24: mol_type = other DNA
cgggatctggtggcgtttaaagtg

SEQ ID NO: 154 - DNA, 21 bp, synthetic construct
  misc_feature 1..21: note = Description of Artificial Sequence: Synthetic primer
  source 1..21: mol_type = other DNA
tgacggcgtttgtcttcaccg

SEQ ID NO: 155 - DNA, 53 bp, synthetic construct
  misc_feature 1..53: note = Description of Artificial Sequence: Synthetic primer
  source 1..53: mol_type = other DNA
acgctatggaactgcaggatgagagcatgaaatggaacggatgcgcgccttat

SEQ ID NO: 156 - DNA, 53 bp, synthetic construct
  misc_feature 1..53: note = Description of Artificial Sequence: Synthetic primer
  source 1..53: mol_type = other DNA
acgctatggaactgcaggatgagagcatctgccccagatcgttggctttttgc

SEQ ID NO: 157 - DNA, 40 bp, synthetic construct
  misc_feature 1..40: note = Description of Artificial Sequence: Synthetic primer
  source 1..40: mol_type = other DNA
tttgaaaacaggatgtagcgatgaccgatattcgcttgta

SEQ ID NO: 158 - DNA, 40 bp, synthetic construct
  misc_feature 1..40: note = Description of Artificial Sequence: Synthetic primer
  source 1..40: mol_type = other DNA
tacaagcgaatatcggtcatcgctacatcctgttttcaaa

SEQ ID NO: 159 - DNA, 40 bp, synthetic construct
  misc_feature 1..40: note = Description of Artificial Sequence: Synthetic primer
  source 1..40: mol_type = other DNA
cgcctgaatactacgattgactggagaatgcgaatgaaca

SEQ ID NO: 160 - DNA, 40 bp, synthetic construct
  misc_feature 1..40: note = Description of Artificial Sequence: Synthetic primer
  source 1..40: mol_type = other DNA
tgttcattcgcattctccagtcaatcgtagtattcaggcg

SEQ ID NO: 161 - DNA, 39 bp, synthetic construct
  misc_feature 1..39: note = Description of Artificial Sequence: Synthetic primer
  source 1..39: mol_type = other DNA
tttgaaaacaggatgtagcggcccgtagtctgcaaatcc

SEQ ID NO: 162 - DNA, 39 bp, synthetic construct
  misc_feature 1..39: note = Description of Artificial Sequence: Synthetic primer
  source 1..39: mol_type = other DNA
ggatttgcagactacgggccgctacatcctgttttcaaa

SEQ ID NO: 163 - DNA, 40 bp, synthetic construct
  misc_feature 1..40: note = Description of Artificial Sequence: Synthetic primer
  source 1..40: mol_type = other DNA
gcattacgctgacttgacggctggagaatgcgaatgaaca

SEQ ID NO: 164 - DNA, 40 bp, synthetic construct
  misc_feature 1..40: note = Description of Artificial Sequence: Synthetic primer
  source 1..40: mol_type = other DNA
tgttcattcgcattctccagccgtcaagtcagcgtaatgc

SEQ ID NO: 165 - DNA, 200 bp, synthetic construct
  misc_feature 1..200: note = Description of Artificial Sequence: Synthetic polynucleotide
  source 1..200: mol_type = other DNA
actggagaatgcgaatgaacagcaataaagagttaatgcagcgccgcagtcaggcgattccccgtggcgttgggcaaattcacccgattttcgctgaccgcgcggaaaactgccgggtgtgggacgttgaaggccgtgagtatcttgatttcgcgggcgggattgcggtgctcaataccgggcacctgcatccgaaggtg

SEQ ID NO: 166 - DNA, 21 bp, synthetic construct
  misc_feature 1..21: note = Description of Artificial Sequence: Synthetic oligonucleotide
  source 1..21: mol_type = other DNA
ggcgaaagagtctgctccggc

SEQ ID NO: 167 - DNA, 23 bp, synthetic construct
  misc_feature 1..23: note = Description of Artificial Sequence: Synthetic oligonucleotide
  source 1..23: mol_type = other DNA
cattcagacacaacgcatcgcgg

SEQ ID NO: 168 - DNA, 43 bp, synthetic construct
  misc_feature 1..43: note = Description of Artificial Sequence: Synthetic oligonucleotide
  source 1..43: mol_type = other DNA
ggcttaccagcaccggtgggttatgcccgtagtctgcaaatcc

SEQ ID NO: 169 - DNA, 42 bp, synthetic construct
  misc_feature 1..42: note = Description of Artificial Sequence: Synthetic oligonucleotide
  source 1..42: mol_type = other DNA
gcattacgctgacttgacgggtgaaaaccgttcgcagcctgg

SEQ ID NO: 170 - DNA, 43 bp, synthetic construct; identical to SEQ ID NO: 168
  misc_feature 1..43: note = Description of Artificial Sequence: Synthetic oligonucleotide
  source 1..43: mol_type = other DNA

SEQ ID NO: 171 - DNA, 42 bp, synthetic construct
  misc_feature 1..42: note = Description of Artificial Sequence: Synthetic oligonucleotide
  source 1..42: mol_type = other DNA
ccaggctgcgaacggttttcacccgtcaagtcagcgtaatgc

SEQ ID NO: 172 - DNA, 46 bp, synthetic construct
  misc_feature 1..46: note = Description of Artificial Sequence: Synthetic oligonucleotide
  source 1..46: mol_type = other DNA
ggcttaccagcaccggtgggttatgtgaaaaccgttcgcagcctgg

SEQ ID NO: 173 - DNA, 46 bp, synthetic construct
  misc_feature 1..46: note = Description of Artificial Sequence: Synthetic oligonucleotide
  source 1..46: mol_type = other DNA
ccaggctgcgaacggttttcacataacccaccggtgctggtaagcc

SEQ ID NO: 174 - DNA, 1317 bp, synthetic construct
  misc_feature 1..1317: note = Description of Artificial Sequence: Synthetic polynucleotide
  source 1..1317: mol_type = other DNA
atggactggaagaagatctatgaagacagaacctgcactgcagatgaagcagtaaagagcattaagtcaggtgaccgcgtgctgtttgcgcactgtgttgctgaaccgccagttctggtagaagcaatggttgcgaatgcagctgcatacaagaatgtcacggtttcacacatggttacccttggtaagggtgaatactcaaaaccagaatataaagaaaactttacttttgaaggttggtttaccagcccttcaacacgtggatccattgcagaaggacacggacagtttgtccctgtattcttccacgaggtaccatctttaatccgtaaagacattttccatgttgatgtattcatggtaatggtatccccgccagatcataacggtttctgctgtgtgggtgtatcttctgactataccatgcaggctatcaaatcagcaaaaattgtactggctgaagtgaatgatcaggtacctgtagtttatggcgatacctttgttcacgttagtgaaatcgacaagttcgttgaaacttcacatccactgccagaaatcggtctgccgaagatcggtgaagtagaagctgctattggtaagcactgcgcttcgctgatcgaagatggttccacattacagcttggtatcggcgctattccggatgctgtactttcacagcttaaggacaagaaacaccttggtatccactctgaaatgatttccgacggtgttgttgatctttacgaagcaggcgttattgactgcagccaaaagtctatcgacaaaggcaaaatggcaataacattcttaatgggaacgaagcgtctttatgatttcgctgcaaacaatccaaaggttgaattaaagccggttgactacatcaatcatccatctgtagttgcacagtgcagcaaaatggtttgcatcaatgcttgcttgcaagttgattttatgggtcagattgtctccgatagtattggcacaaagcagttctccggcgtaggcggtcaggttgacttcgtacgcggtgcatccatgtctattgacggcaaaggtaaagcgatcatcgcgatgccttccgttgcaaagaagaaagatggcagtatgatttcgaagatcgttccattcatcgatcacggtgcagctgtaactacatcccgtaacgatgcggactatgtcgtaacggaatatggtattgctgaaatgaagggtaagtcgttacaggaccgcgcacgcgcgttaatcaatattgcccaccctgatttcaaagatgaattaaaggctgaatttgaaaagcgcttcaacgcggcattctaa

SEQ ID NO: 175 - DNA, 1308 bp, synthetic construct
  misc_feature 1..1308: note = Description of Artificial Sequence: Synthetic polynucleotide
  source 1..1308: mol_type = other DNA
atgagctggcaagaactgtatcaaagtaaattatgttcagccacagaagcggtaaaacagattaaaaacggtgataccgtggtatttgcccattgtgtaggtgaaccgcctgcactggtggaggcgatgattgaaaatgctgaacaatataaagatgttgagattaaacatatggttagcctgggtagtggtggttatactgcgaaagggatggaagcgcattttcgcgtaaatccaatgtttgtcagcggcaatgtacgtaaggcgattgaaaatggcgatggtgattttacacctgcattcttccatgaagtaccaaagttgctgcgtgaaaaacgtctgaaatgtgatgttgttctggcacaggtaacgccaccagatgaacatggttattgttcgctgggaacaagcgttgattatacctatgaagccattaaaaccgcgcgcaccgtaattgttcaggtgaatgaccagtttcctcgcacctatggtgaggtggtgcatgtcagcgagtttgactatatcgttgaaaaatcacaaccgctgtttgaactgcaacctgcaaagattggcgaagttgaagaagcgattggtaaaaattgtgcctcgctgattgaagatggtagcacgttacagctggggattggtgggattccggatgcggtgatgttatttctgactgataaaaaagatttagggattcatagcgaaatgattagcgatggcacgctggcgctttatgaaaaaggtgttattaatggtaaatataaaaattttgataaagaaaaaatgacggttaccttcctgatgggtactaaaaaactgtatgactttgccaataataacccggcagtagaggtaaaaccggtagactatgtgaatcatccggcaattatcatgaaacaacataagatggtttctattaatagcgccattcaggttgatttaatggggcaggtggttgcagaggcgatgggactgcgccaattttccggtgttggcggtcaggttgactttattcgtggcgtgtcgatgggtgaagatggcaaggcgattatcgcgatgccttcaatcactacaaaaaaagatggtacggtaattagcaaaatcgtctctattgtcgatgaaggtgcaccgattaccacctcacgtaatgatgttgattatattgtcacagaatacggtattgcagaattaaaaggcaaatcgctgcgtgaacgcgcacgtaatctgattaatattgctcatccatcggtacgtgaatcgctggcagtagaatttgaaaagcgctttaaagagaaatattaa

SEQ ID NO: 176 - DNA, 1539 bp, synthetic construct
  misc_feature 1..1539: note = Description of Artificial Sequence: Synthetic polynucleotide
  source 1..1539: mol_type = other DNA
atggatttgaagctgaccgatgcggacgttgaggcaatcgtagcgcaagtcatggctaacattgagcgccgtctgggtagcgcggaagcgggcagcgcagcatccgctgcgtcgccggcaccggctgcgccggttcgtaccgctccgggtggcagcccggcagccagcccgcgtccggagtacggtgtttttgatcgtgcggaggatgcagtcgccgctgccgccgaagctcaggaagccttcctgcgccagtgtcgtctgcaagaccgtgagcgcattctgcgtgccatccgtgaagagactctggcacgcaaggaagaattggcacgcctgatttgggaggaaacgaagctgggtcgcttggaacacaaaattgcaaagctggaattgacggcgctgaaaaccccgggtacggaggatctgcgcaccgaggcatttagcggcgacaacggcctgaccatcgtcgaacatgcgccgtacggtgtgattggcgcggttaccccggttacgaaccctgcggagactattatcaacaacgcgatcggcatgctggcaagcggtaatgcagtggtgttcaacgtgcaccctagcgctaaacgttgctccgcgtataccgtccagatgatcaataaagcagtgatggcagcgggtggtccgccgaatctggttacgatggttcgcgagccgaccatggaaaccctgaatgcgatcatccgcagcccgagcgtgaagctgctggtgggcaccggcggtccgggtctggttagcaccctgctgcgctctggtaagaaagcgattggtgcgggtgcgggcaatccgccggtcgttgtggatgataccgccgacctggagcatgcggcaaaggaaatcatcaaaggcgcgtcctttgacaacaatattctgtgtattgcggagaaagaggtttttgtggttgataaagcggccgacggtctgatctatcacatgctggataacggcgcatacatgctgggtcgtgacgagctggagaaggtgatgcaattcgcgctgaccgcggacgaaagccagggcggtgcgggttgctctattgatccgcgtcgtgcgtggcatgtgactaaggagtgggtgggcaaagatgcgcgcttgttcctggagaagattggtgtcaagactgaccgtccggttgacttgctgttgtgcgaggttgactttgaccacccgttcgtgcagctggaacaaatgatgccagtactgccgattgttcgtgtccgtgacctggatgaagccatcggtatggccgtccgtgcggagcacggcaatcgtcacaccgcaattatgcatagccgtaacgtggacaatctgacccgttttgcccgtgccattgccacgaccatcttcgtcaaaaacgcaagcagcttggcgggtgttggttatggcggtgaaggttttaccaccatgacgatcgccggtccaacgggcgagggtctgacgtcggctcgtacgttcacgcgcaaagtccgctgtgtcctggcggacggcggtttccgtatcgttggttaa

SEQ ID NO: 177 - DNA, 792 bp, Yersinia intermedia
  source 1..792: mol_type = other DNA
atgaccgatattcgcttgtatatgtttcaaactggctcgctgaagtgcaaagtccacaatatcaaaatgaatcaaggaggtggagcggattatgaaatccctgtcccgttcttcctgctgacccatcctgatggtcacacgctgattgatggcgggaatgctgtcgaaaccgccactgatcccaaagggtattggggcgggattaccgaggtttattggccggtaatgcgtgaagatgagggctgtgtagcgcagcttaaaaaaatggggatcaatcccgaagatatccggtatgtcttgcagtcgcacttgcatcttgatcataccggtgcgattggccgattccccaatgcgacacacatcgttcagcgccgtgagtacgagtatgcctttactcctgactggtttgctgcgggaggctatatcagaaacgactttgaccggccgggtctgaaatgggcgtttctggaaggcgaaaacaatgatttttatgatatctatggcgacggtacgctgaaaacagtatttacacccggccattcgcctgggcatcagtcaatactggtcaccttgcccaactcaggtccgatgctactgactattgatgcagcatacaccacggatcactgggaagaaaaagctctgccaggatttatgtcctcagccgttgagacggtgcgttcggtgcagaagatgcgcatgttggctagccgcacaagtgcgcaagtggtgacaggccatgacccggatgcctggcaaacgttcaggcacgcgcctgaatactacgattga

SEQ ID NO: 178 - DNA, 771 bp, Clostridium beijerinckii
  source 1..771: mol_type = other DNA
atgcttcagtcgggtacgctgaaatgcaaggtacacaacattaagatgaaccaggggaacggtgcagactatgagatccccgttccgtttttcctgattacccatccggccgggcacaccgtgatcgacggcggcaacgcgattgaagttgcaacagatccgcgtggccattggggcggcatctgcgatgtctattggccagtactggacaaggaccagggctgcgttgaccagatcaaggcgcttggtttcgatccggccgatgtcaaatatgttgtgcagtcgcacctgcatctcgatcataccggcgccatcggtcgcttccccaacgcaacccacatcgtgcagcgctctgaatatgaatatgcctttacgcccgactggtttgctggcggcggctatatccgcaaggacttcgacaagccgggtctgaagtggcagttcctcaacggtgcgcaggacgattattacgatgtttacggcgacggcacgctcaccacgatcttcacgcccggtcatgcgcccggccaccagtccttcctggtgcgcctgccaaacagcaaaccgcttctcctgacgatcgatgctgcctacacactggaccactgggaagagaaggctttgcctggcttccttgcctcgaccgttgacacggtccgttctgttcagaagctccgcacctatgccgaaaagcatgatgcgacagtcgtcaccggccatgaccctgacgcctgggcgaacttcaagaaggctcccgaattttacgcgtaa

SEQ ID NO: 179 - protein, 263 aa, Yersinia intermedia
MTDIRLYMFQTGSLKCKVHNIKMNQGGGADYEIPVPFFLLTHPDGHTLIDGGNAVETATDPKGYWGGITEVYWPVMREDEGCVAQLKKMGINPEDIRYVLQSHLHLDHTGAIGRFPNATHIVQRREYEYAFTPDWFAAGGYIRNDFDRPGLKWAFLEGENNDFYDIYGDGTLKTVFTPGHSPGHQSILVTLPNSGPMLLTIDAAYTTDHWEEKALPGFMSSAVETVRSVQKMRMLASRTSAQVVTGHDPDAWQTFRHAPEYYD

SEQ ID NO: 180 - protein, 256 aa, Clostridium beijerinckii
MLQSGTLKCKVHNIKMNQGNGADYEIPVPFFLITHPAGHTVIDGGNAIEVATDPRGHWGGICDVYWPVLDKDQGCVDQIKALGFDPADVKYVVQSHLHLDHTGAIGRFPNATHIVQRSEYEYAFTPDWFAGGGYIRKDFDKPGLKWQFLNGAQDDYYDVYGDGTLTTIFTPGHAPGHQSFLVRLPNSKPLLLTIDAAYTLDHWEEKALPGFLASTVDTVRSVQKLRTYAEKHDATVVTGHDPDAWANFKKAPEFYA

SEQ ID NO: 181 - protein, 8 aa, synthetic construct
  REGION 1..8: note = Description of Artificial Sequence: Synthetic peptide
EGYDVFEA

SEQ ID NO: 182 - DNA, 69 bp, synthetic construct
  misc_feature 1..69: note = Description of Artificial Sequence: Synthetic primer
  source 1..69: mol_type = other DNA
atgatgttttcgaagcggaaggctatgatgttttcgaagcgacagatggcgcggaaatgcatcagatcc

SEQ ID NO: 183 - DNA, 71 bp, synthetic construct
  misc_feature 1..71: note = Description of Artificial Sequence: Synthetic primer
  source 1..71: mol_type = other DNA
tttccgcgccatctgtcgcttcgaaaacatcatagccttccgcttcgaaaacatcatagccttccgcttcg

SEQ ID NO: 184 - DNA, 24 bp, synthetic construct
  misc_feature 1..24: note = Description of Artificial Sequence: Synthetic primer
  source 1..24: mol_type = other DNA
taatgatatttcgcccgactgcgc

SEQ ID NO: 185 - DNA, 26 bp, synthetic construct
  misc_feature 1..26: note = Description of Artificial Sequence: Synthetic primer
  source 1..26: mol_type = other DNA
aatgaacggtatttatttgcaagcgg

SEQ ID NO: 186 - DNA, 1963 bp, synthetic construct
  misc_feature 1..1963: note = Description of Artificial Sequence: Synthetic polynucleotide
  source 1..1963: mol_type = other DNA
taatgatatttcgcccgactgcgcgcagtggtggcgacagtgaaatcgacatcgtgtaacgattcagccaatgtcgggaaaactttaatattatcaataatatcaccagatccatgtgcgacccagcgggtggctggctccaggtgtgcctgactatcgacaatccgcagatcgctaaaccccatcgttttcattgcccgcgccgctgccccaatattttctgctctggcgggtgcgaccagaataatcgttatacgcatattgccactcttcttgatcaaataaccgcgaaccgggtgatcactgtcaacttattacgcggtgcgaatttacaaattcttaacgtaagtcgcagaaaaagccctttacttagcttaaaaaaggctaaactatttcctgactgtactaacggttgagttgttaaaaaatgctacatatccttctgtttacttaggataattttataaaaaataaatctcgacaattggattcaccacgtttattagttgtatgatgcaactagttggattattaaaataatgtgacgaaagctagcatttagatacgatgatttcatcaaactgttaacgtgctacaattgaacttgatatatgtcaacgaagcgtagttttattgggtgtccggcccctcttagcctgttatgttgctgttaaaatggttaggatgacagccgtttttgacactgtcgggtcctgagggaaagtacccacgaccaagctaatgatgttgttgacgttgatggaaagtgcatcaagaacgcaattacgtactttagtcatgttacgccgatcatgttaatttgcagcatgcatcagcaggtcagggacttttgtacttcctgtttcgatttagttggcaatttaggtagcaaacatgcagaccccgcacattcttatcgttgaagacgagttggtaacacgcaacacgttgaaaagtattttcgaagcggaaggctatgatgttttcgaagcggaaggctatgatgttttcgaagcgacagatggcgcggaaatgcatcagatcctctctgaatatgacatcaacctggtgatcatggatatcaatctgccgggtaagaacggtcttctgttagcgcgtgaactgcgcgagcaggcgaatgttgcgttgatgttcctgactggccgtgacaacgaagtcgataaaattctcggcctcgaaatcggtgcagatgactacatcaccaaaccgttcaacccgcgtgaactgacgattcgtgcacgcaacctactgtcccgtaccatgaatctgggtactgtcagcgaagaacgtcgtagcgttgaaagctacaagttcaatggttgggaactggacatcaacagccgttcgttgatcggccctgatggcgagcagtacaagctgccgcgcagcgagttccgcgccatgcttcacttctgtgaaaacccaggcaaaattcagtcccgtgctgaactgctgaagaaaatgaccggccgtgagctgaaaccgcacgaccgtactgtagacgtgacgatccgccgtattcgtaaacatttcgaatctacgccggatacgccggaaatcatcgccaccattcacggtgaaggttatcgcttctgcggtgatctggaagattaatcggctttaccaccgtcaaaaaaaacggcgctttttagcgccgtttttatttttcaaccttatttccagatacgtaactcatcgtccgttgtaacttctttactggctttcattttcggcagtgaaaacgcataccagtcgatattacgggtcacaaacatcatgccggccagcgccaccaccagcacactggttcccaacaacagcgcgctatcggcagagttgagcagtccccacatcacaccatccagcaacaacagcgcgagggtaaacaacatgctgttgcaccaacctttcaataccgcttgcaaataaataccgttcatta

SEQ ID NO: 187 - DNA, 498 bp, synthetic construct
  misc_feature 1..498: note = Description of Artificial Sequence: Synthetic polynucleotide
  source 1..498: mol_type = other DNA
atgggtttgttcgataaactgaaatctctggtttccgacgacaagaaggataccggaactattgagatcattgctccgctctctggcgagatcgtcaatatcgaagacgtgccggatgtcgtttttgcggaaaaaatcgttggtgatggtattgctatcaaaccaacgggtaacaaaatggtcgcgccagtagacggcaccattggtaaaatctttgaaaccaaccacgcattctctatcgaatctgatagcggcgttgaactgttcgtccacttcggtatcgacaccgttgaactgaaaggcgaaggcttcaagcgtattgctgaagaaggtcagcgcgtgaaagttggcgatactgtcattgaatttgatctgccgctgctggaagagaaagccaagtctaccctgactccggttgttatctccaacatggacgaaatcaaagaactgatcaaactgtccggtagcgtaaccgtgggtgaaacccataagcgctaa

SEQ ID NO: 188 - protein, 431 aa, Porphyromonas gingivalis
MQWQELYRQRVCSADEAVVDSLKPGTKVVFGHAAAAPVRFSQAMYRQREKLENITVFHMLYFGDAPHLAPEMRSHVHPTLNFLEGNSRPASRDRRVDFIPCHFHEVPELFRQGFFPLDVAVVQVSTPNEEGYCSFGVSCDYTKAAAECAPVVVAEVNKQMPFIGGENLIHISKLTHIIEVDEPIAEVLPPAISDLELRIGQNCASLIKDGDTLQLGIGGIPDAVLRALEGHKDLGIHTEMFTDGVMRMIRKGIINGKKKTLHPEKVVTSLIFGSKELYDFVNNNPVIECYPVDYINNPDVIGKNDRMVSINSCLEMDLMGQAASESIGYEQFSGSGGQVDFLRGAKRSKGGISIMAFPSTAKKGTESRIVPILKEGACVTTGRNEVDYVVTEYGVARLRGATLRQRAEALTAIAHPDFRPALEEEIRRRFE

SEQ ID NO: 189 - protein, 431 aa, Roseiflexus castenholzii
MPRSLTAGRREMTAEEAVELIESNHRVYIGGGCGVPIPLLDALVARAPELRNVEIIHMLTAGEDPTTAPELAASFRHNALFIGHNTRRAVNEGRADFTPIFLGEIPKLFRQGILPLDVAMIQVSPPDRHGFCSLGVEVGCTLPAARTAKIVIAEVNARMPRTLGDSFIHISRLTALVESDRPLLELPQGETNSVAQAIGRHIAELIPDGATLQLGIGAIPDAVLSNLHGKRHLGIHTELFSDGVIDLVEAGVIDGELKTIHQGKVVAGFILGSQRCFDWAHNNAMVEMHPTDYVNDPFVIAQHKNMVAVNSALQVDLTGQVCADSIGTRLYSGVGGQVDFIRGASRSEGGIPIIAISSTARDGTISRIVPTLDVGAGVVTSRYDVHFVVTEYGVADLYGRTLAQRARALINIAHPAFRDQLTEAAKKLHYI

SEQ ID NO: 190 - protein, 436 aa, Clostridium beijerinckii
MNWQEEYKRRLCSAEEAVQHIHSGDTVFLGHAVAESSLLVDAMVQNATAYQDVRIIHMTNLGSGAYTQPGMESHFHVSPFFLGATVRAAVAEGRGDFIPCFFHEIPQLIRERRVPCDVLMTQVSPSDEKGYCSLGVSADYTYQALKSARTVIAHVNDQYPYTFGTKVHVTELDYIVEQSAPLYESKPPKIGEVEEKIGRCCASLIRDGATLQLGIGAIPDAVMMFLGDKHDLGIHSEMISDGTMELYQKGVITNQRKNLDRGKMTITFAMGTKKLYQFLDKNPDVLVKPVDYVNHPVTIMKEHDMISINSAIQVDLQGQVDAEAIGLRQFSGVGGQVDFIRGAAMGGGTSIIAMPSTTQKKDGTLISKIVPFLDQGAPVTTSRNDVDYIITEYGIAHLKGETLRDRAWALISISHPSVRDSLAEEFERRFHQNYRA

SEQ ID NO: 191 - protein, 436 aa, Fusobacterium necrophorum
MSKNWREVYKDKIVTADEAVKQIKSGDRLIFSHACGEAQEITDALLRNKESYEKVEIIHLVPMGKGEYAQEENQKYFYHNSFFGGGSTRKAINGTYGDFTPSFFFEIPNLFRKNGKLPLDVAIIQVSAPDEHGYCSYGISCDYTKGAAENAKIVIAQVNKYMPRTLGENFTHLSAIDSIVEYDQPILQLNPPKIGEVEKKIGEYCAGLIQDGDTLQLGIGAIPDAVLTFLKEKKHLGIHSEMISDGVVDLILAGVIDNSQKTIHKNKCIVSFLMGSQKLYDYVHNNPGVELYPVDYVNHPFVIAQNDNMVSINSALQVDLMGQVNAESMGAKQFSGTGGQVDFVRGAAMSKGGRSIIAMPSTAAKGTISKIVMNLDVGATVTTSRNDVDYIITEYGIAELKGKTLRERAKALIAIAHPDFREQLTKQALEKFQRLE

SEQ ID NO: 192 - protein, 429 aa, Clostridium kluyveri
MEWEEIYKEKLVTAEKAVSKIENHSRVVFAHAVGEPVDLVNALVKNKDNYIGLEIVHMVAMGKGVYTKEGMQRHFRHNALFVGGSTRDAVNSGRAVYTPCFFYEVPSLFKEKRLPVDVALIQVSEPDKYGYCSFGVSNDYTKPAAESAKLVIAEVNKNMPRTLGDSFIHVSDIDYIVEASHPLLELQPPKLGDVEKAIGENCASLIEDGATLQLGIGAIPDAVLLFLKNKKNLGIHSEMISDGVMELVKAGVINNKKKTLHPGKIVVTFLMGTKKLYDFVNNNPMVETYSVDYVNNPLVIMKNDNMVSINSCVQVDLMGQVCSESIGLKQISGVGGQVDFIRGANLSKGGKAIIAIPSTAGKGKVSRITPLLDTGAAVTTSRNEVDYVVTEYGVAHLKGKTLRNRARALINIAHPKFRESLMNEFKKRF

SEQ ID NO: 193 - protein, 436 aa, Clostridium beijerinckii
MDWKKIYEDRTCTADEAVKSIKSGDRVLFAHCVAEPPVLVEAMVANAAAYKNVTVSQMVTLGKGEYSKPEYKENFTFEGWFTSPSTRGSIAEGHGQFVPVFFHEVPSLIRKDIFHVDVFMMVSPPDHNGFCCVGVSSDYTMQAIKSAKIVLAEVNDQVPVVYGDTFVHVSEIDKFVETSHPLPEIGLPKIGEVEAAIGKHCASLIEDGSTLQLGIGAIPDAVLSQLKDKRHLGIHSEMISDGVVDLYEAGVIDCSQKSIDKGKMAITFLMGTKRLYDFAANNPKVELKPVDYINHPSVVAQCSKMVCINSCLQVDFMGQIVSDSIGTKQYSGVGGQVDFVRGASMSIDGKGKAIIAMPSVAKKKDGSMISKIVPFIDHGAAVTTSRNDADYVVTEYGIAMKGKSLQDRARALINIAHPDFKDELKAEFEKRFNAAF

SEQ ID NO: 194 - protein, 436 aa, Clostridium beijerinckii
MSKISWKDLYKSKVVTADEAVRKIKSNDRVVTGHACGEPKEIIDAMVRNKDLYENVEIVHMVSMGKSEYCKPEMAVNFRHNSIFAGGTTREAIFDGRADFTPCFFSEVPKMFREGTLPVDVALVQLSVPDEHGYCSFGVSNDYTKPAAEAAKIVIAELNEKMPRTLGDSFIHVSDIDYIVETSNDIIELKPPKIGEVEKAIGENCAKLIEDGSTLQLGIGAIPDAVLLFLKGKKDLGIHSEMISDGVVELIEAGVITNKAKTLHPGKSVVTFLMGTKRLYDYVNGNPSVAMYPVDYVNNPCVIAENYKMVSINSCIQVDLMGQVAADTIGLKQFSGVGGQVDFVRGAAMAKGGKSIIAMPSTASKGKLSRIVPILDEGATVTTSRNDIHYVVTEFGIAELKGKTLKERAKALINVAHPDFRDALIKEWEKRFKVKF

SEQ ID NO: 195 - protein, 438 aa, Eubacterium saphenum
MDWKKIYEEKRCTAKEAVQSIKSNDRVVFAHAVAEPTSLVDAMVENKEAYENVTISHMFSFGKGEYSLPENSKHFRYEGWFNSPNTRKSTELGHGDYVPAFFHQVPSLIRRGVLPVDVVMVSLSKPDAHGYCSTGVSSDYTMQAIKSAKVVLAEINDQVPKTFGESFVHISEIDKLVEHSHPLFELGLPKIGEVEEAIGKHCASLIEDGATLQLGIGAIPDAVLAQLKHKKDLGIHSEMISDGAVELFEAGVINNSKKSIDKGKMVVTFLMGTKRLYDFVNENPAVELRPVDYVNHPEVIAKSSNLVCINACLQVDFMGQVVSDTIGTRQFSGVGGQVDFVRGAAMAHDEKAKAIIAMPSVAVKKDGTKISKIVPFIDHGAAVTTSRHDTDYIVTEYGIAEMVGKTMKDRARSLINIAHPDFRDELKAEFEKRFNVKF

SEQ ID NO: 196 - protein, 433 aa, Clostridium sp.
MSWQELYQQKRMTAEQAVSHIKSGDRVVVAHATGEPSALIDAMVKNADAYRNVEIVHMVAMGKGEYCLPEYTENFRHNSLFLGASTRDAIAKGRGDFTPVYFSQIPDLLREELHPNVALLHLSPPDEHGYCSYGVSVDYTKPAAEIADLRIAQINPNMPRTLGDSFIHVSELDCIVEVDDPIIELGQAKIGETECAIGEHCASLIQDGDCLQLGIGAIPDAVLLFLKEKKDLGIHSEMFSDGVVELVEAGVITNRRKTLHKGQCVVTFLMGSKRLYDYVNNNPSVAMYPVDYVNNPAVIAKNDNLVSINSCIQVDLMGQVVSDSIGPRQFSGVGGQVDFVRGANLSRGGKSIMAMPSSLVKKDGTRVSKIAVQIDAGAAVTTSRYDVNYIVTEFGVAKLKGKTLRDRAKALIEIAHPDFREELMEEFHRRFGV

SEQ ID NO: 197 - protein, 438 aa, Clostridium beijerinckii
MDWKKIYEDRTCTADEAVKSIKSGDRVLFAHCVAEPPVLVEAMVANAAAYKNVTVSHMVTLGKGEYSKPEYKENFTFEGWFTSPSTRGSIAEGHGQFVPVFFHEVPSLIRKDIFHVDVFMVMVSPPDHNGFCCVGVSSDYTMQAIKSAKIVLAEVNDQVPVVYGDTFVHVSEIDKFVETSHPLPEIGLPKIGEVEAAIGKHCASLIEDGSTLQLGIGAIPDAVLSQLKDKKHLGIHSEMISDGVVDLYEAGVIDCSQKSIDKGKMAITFLMGTKRLYDFAANNPKVELKPVDYINHPSVVAQCSKMVCINACLQVDFMGQIVSDSIGTKQFSGVGGQVDFVRGASMSIDGKGKAIIAMPSVAKKKDGSMISKIVPFIDHGAAVTTSRNDADYVVTEYGIAEMKGKSLQDRARALINIAHPDFKDELKAEFEKRFNAAF

SEQ ID NO: 198 - protein, 433 aa, Clostridium carboxidivorans
MDWKKLYKSKLVSAKEAVSNIKSNSRVVVSIAVAEPTELIDALVANKENYENVEVVHMVDMGKSEYAQEGMEKYFKYNSIFVGASTKDAVNSGRSDFTPCCFYELPRLFEEGYLPVDVVLIQVSKPDKHGYCSFGVSNDYTKPAADCAKMVIAEVNENMPRVLGDSFIHISDIDYIVETSHPIMELKQPKIGKIEEAIGEYCASLIEDGSTLQLGIGAIPDAVLLFLKDKKDLGIHSEMISDGVVDLVESGVINNKEKTLNPGKIVVTFFMGTKKLYDFIDDNPMVESYPVSYVNDPTVIMKNSKMISINSCVEVDLMGQVCSESIGMNQISGIGGQVDFIRGANMCKDGKAIIAIPSTAAKGKVSRIVPLIEKGTPITTSRTDVDYIITEYGIARLKSKSLKERARALINIAHPDFRAWLIDEYEKRFKTKF

SEQ ID NO: 199 - protein, 431 aa, synthetic construct
  REGION 1..431: note = Description of Artificial Sequence: Synthetic polypeptide
MQWQELYRQRVCSADEAVVDSLKPGTKVVLGGAAAAPVRFSQAMYRQREKLENITVFQMLYFGDAPHLAPEMRSHVHPTLNFLEGNSRPASRDRRVDFIPCHFHEVPYLFRQGFFPLDVAVVQVSTPNEEGYCSFGVSCDYTKAAAECAPVVVAEVNKQMPFIGGENLIHISKLTHIIEVDEPIAEVLPPAISDLELRIGQNCASLIKDGDTLQLGIGGIPDAVLRALEGHRDLGIHTEMFTDGVMRMIRKGIINGKKKTLHPEKVVTSLIFGSKELYDFVNNNPVIECYPVDYINNPDVIGKNDRMVSINSCVEMDLMGQAASESIGYEQYSGSGGQVDFLRGAKRSKGGISIMAFPSTAKKGTESRIVPILKEGACVTTGRNEVDYVVTEYGVARLRGATLRQRAEALTAIAHPDFRPALEEEIRRRFE

SEQ ID NO: 200 - protein, 431 aa, synthetic construct
  REGION 1..431: note = Description of Artificial Sequence: Synthetic polypeptide
MPRSLTAGRREMTAEEAVELIESNHRVYLGHGAGVPIPLLDALVARAPELRNVEIIQMLTAGEDPTTAPELAASFRHNALFIGHNTRRAVNEGRADFTPIFLGEIPYLFRQGILPLDVAMIQVSPPDRHGFCSLGVEVGCTLPAARTAKIVIAEVNARMPRTLGDSFIHISRLTALVESDRPLLELPQGETNSVAQAIGRHIAELIPDGATLQLGIGAIPDAVLSNLHGKRHLGIHTELFSDGVIDLVEAGVIDGELKTIHQGKVVAGFILGSQRCFDWAHNNAMVEMHPTDYVNDPFVIAQHKNMVAVNSAVQVDLTGQVCADSIGTRLYSGVGGQVDFIRGASRSEGGIPIIAISSTARDGTISRIVPTLDVGAGVVTSRYDVHFVVTEYGVADLYGRTLAQRARALINIAHPAFRDQLTEAAKKLHYI

SEQ ID NO: 201 - protein, 436 aa, synthetic construct
  REGION 1..436: note = Description of Artificial Sequence: Synthetic polypeptide
MNWQEEYKRRLCSAEEAVQHIHSGDTVFLGGAAAESSLLVDAMVQNATAYQDVRIIQMTNLGSGAYTQPGMESHFHVSPFFLGATVRAAVAEGRGDFIPCFFHEIPYLIRERRVPCDVLMTQVSPSDEKGYCSLGVSADYTYQALKSARTVIAHVNDQYPYTFGTKVHVTELDYIVEQSAPLYESKPPKIGEVEEKIGRCCASLIRDGATLQLGIGAIPDAVMMFLGDKRDLGIHSEMISDGTMELYQKGVITNQRKNLDRGKMTITFAMGTKKLYQFLDKNPDVLVKPVDYVNHPVTIMKEHDMISINSAVQVDLQGQVDAEAIGLRQYSGVGGQVDFIRGAAMGGGTSIIAMPSTTQKKDGTLISKIVPFLDQGAPVTTSRNDVDYIITEYGIAHLKGETLRDRAWALISISHPSVRDSLAEEFERRFHQNYRA

SEQ ID NO: 202 - protein, 436 aa, synthetic construct
  REGION 1..436: note = Description of Artificial Sequence: Synthetic polypeptide
MSKNWREVYKDKIVTADEAVKQIKSGDRLILSGAAGEAQEITDALLRNKESYEKVEIIQLVPMGKGEYAQEENQKYFYHNSFFGGGSTRKAINGTYGDFTPSFFFEIPYLFRKNGKLPLDVAIIQVSAPDEHGYCSYGISCDYTKGAAENAKIVIAQVNKYMPRTLGENFTHLSAIDSIVEYDQPILQLNPPKIGEVEKKIGEYCAGLIQDGDTLQLGIGAIPDAVLTFLKEKRHLGIHSEMISDGVVDLILAGVIDNSQKTIHKNKCIVSFLMGSQKLYDYVHNNPGVELYPVDYVNHPFVIAQNDNMVSINSAVQVDLMGQVNAESMGAKQYSGTGGQVDFVRGAAMSKGGRSIIAMPSTAAKGTISKIVMNLDVGATVTTSRNDVDYIITEYGIAELKGKTLRERAKALIAIAHPDFREQLTKQALEKFQRLE

SEQ ID NO: 203 - protein, 429 aa, synthetic construct
  REGION 1..429: note = Description of Artificial Sequence: Synthetic polypeptide
MEWEEIYKEKLVTAEKAVSKIENHSRVVLAGAAGEPVDLVNALVKNKDNYIGLEIVQMVAMGKGVYTKEGMQRHFRHNALFVGGSTRDAVNSGRAVYTPCFFYEVPYLFKEKRLPVDVALIQVSEPDKYGYCSFGVSNDYTKPAAESAKLVIAEVNKNMPRTLGDSFIHVSDIDYIVEASHPLLELQPPKLGDVEKAIGENCASLIEDGATLQLGIGAIPDAVLLFLKNKRNLGIHSEMISDGVMELVKAGVINNKKKTLHPGKIVVTFLMGTKKLYDFVNNNPMVETYSVDYVNNPLVIMKNDNMVSINSCVQVDLMGQVCSESIGLKQYSGVGGQVDFIRGANLSKGGKAIIAIPSTAGKGKVSRITPLLDTGAAVTTSRNEVDYVVTEYGVAHLKGKTLRNRARALINIAHPKFRESLMNEFKKRF

SEQ ID NO: 204 - protein, 438 aa, synthetic construct
  REGION 1..438: note = Description of Artificial Sequence: Synthetic polypeptide
MDWKKIYEDRTCTADEAVKSIKSGDRVLLAGCAAEPPVLVEAMVANAAAYKNVTVSQMVTLGKGEYSKPEYKENFTFEGWFTSPSTRGSIAEGHGQFVPVFFHEVPYLIRKDIFHVDVFMVMVSPPDHNGFCCVGVSSDYTMQAIKSAKIVLAEVNDQVPVVYGDTFVHVSEIDKFVETSHPLPEIGLPKIGEVEAAIGKHCASLIEDGSTLQLGIGAIPDAVLSQLKDKRHLGIHSEMISDGVVDLYEAGVIDCSQKSIDKGKMAITFLMGTKRLYDFAANNPKVELKPVDYINHPSVVAQCSKMVCINSCVQVDFMGQIVSDSIGTKQYSGVGGQVDFVRGASMSIDGKGKAIIAMPSVAKKKDGSMISKIVPFIDHGAAVTTSRNDADYVVTEYGIAEMKGKSLQDRARALINIAHPDFKDELKAEFEKRFNAAF

SEQ ID NO: 205 - protein, 436 aa, synthetic construct
  REGION 1..436: note = Description of Artificial Sequence: Synthetic polypeptide
MSKISWKDLYKSKVVTADEAVRKIKSNDRVVLGGAAGEPKEIIDAMVRNKDLYENVEIVQMVSMGKSEYCKPEMAVNFRHNSIFAGGTTREAIFDGRADFTPCFFSEVPYMFREGTLPVDVALVQLSVPDEHGYCSFGVSNDYTKPAAEAAKIVIAELNEKMPRTLGDSFIHVSDIDYIVETSNDIIELKPPKIGEVEKAIGENCAKLIEDGSTLQLGIGAIPDAVLLFLKGKRDLGIHSEMISDGVVELIEAGVITNKAKTLHPGKSVVTFLMGTKRLYDYVNGNPSVAMYPVDYVNNPCVIAENYKMVSINSCVQVDLMGQVAADTIGLKQYSGVGGQVDFVRGAAMAKGGKSIIAMPSTASKGKLSRIVPILDEGATVTTSRNDIHYVVTEFGIAELKGKTLKERAKALINVAHPDFRDALIKEWEKRFKVKF

SEQ ID NO: 206 - protein, 438 aa, synthetic construct
  REGION 1..438: note = Description of Artificial Sequence: Synthetic polypeptide
MDWKKIYEEKRCTAKEAVQSIKSNDRVVLAGAAAEPTSLVDAMVENKEAYENVTISQMFSFGKGEYSLPENSKHFRYEGWFNSPNTRKSTELGHGDYVPAFFHQVPYLIRRGVLPVDVVMVSLSKPDAHGYCSTGVSSDYTMQAIKSAKVVLAEINDQVPKTFGESFVHISEIDKLVEHSHPLFELGLPKIGEVEEAIGKHCASLIEDGATLQLGIGAIPDAVLAQLKHKRDLGIHSEMISDGAVELFEAGVINNSKKSIDKGKMVVTFLMGTKRLYDFVNENPAVELRPVDYVNHPEVIAKSSNLVCINSCLQVDFMGQVVSDTIGTRQYSGVGGQVDFVRGAAMAHDEKAKAIIAMPSVAVKKDGTKISKIVPFIDHGAAVTTSRHDTDYIVTEYGIAEMVGKTMKDRARSLINIAHPDFRDELKAEFEKRFNVKF

SEQ ID NO: 207 - protein, 433 aa, synthetic construct
  REGION 1..433: note = Description of Artificial Sequence: Synthetic polypeptide
MSWQELYQQKRMTAEQAVSHIKSGDRVVLAGAAGEPSALIDAMVKNADAYRNVEIVQMVAMGKGEYCLPEYTENFRHNSLFLGASTRDAIAKGRGDFTPVYFSQIPYLLREELHPNVALLHLSPPDEHGYCSYGVSVDYTKPAAEIADLRIAQINPNMPRTLGDSFIHVSELDCIVEVDDPIIELGQAKIGETECAIGEHCASLIQDGDCLQLGIGAIPDAVLLFLKEKRDLGIHSEMFSDGVVELVEAGVITNRRKTLHKGQCVVTFLMGSKRLYDYVNNNPSVAMYPVDYVNNPAVIAKNDNLVSINSCVQVDLMGQVVSDSIGPRQYSGVGGQVDFVRGANLSRGGKSIMAMPSSLVKKDGTRVSKIAVQIDAGAAVTTSRYDVNYIVTEFGVAKLKGKTLRDRAKALIEIAHPDFREELMEEFHRRFGV

SEQ ID NO: 208 - protein, 438 aa, synthetic construct
  REGION 1..438: note = Description of Artificial Sequence: Synthetic polypeptide
MDWKKIYEDRTCTADEAVKSIKSGDRVLLAGCAAEPPVLVEAMVANAAAYKNVTVSQMVTLGKGEYSKPEYKENFTFEGWFTSPSTRGSIAEGHGQFVPVFFHEVPYLIRKDIFHVDVFMVMVSPPDHNGFCCVGVSSDYTMQAIKSAKIVLAEVNDQVPVVYGDTFVHVSEIDKFVETSHPLPEIGLPKIGEVEAAIGKHCASLIEDGSTLQLGIGAIPDAVLSQLKDKRHLGIHSEMISDGVVDLYEAGVIDCSQKSIDKGKMAITFLMGTKRLYDFAANNPKVELKPVDYINHPSVVAQCSKMVCINSCVQVDFMGQIVSDSIGTKQFSGVGGQVDFVRGASMSIDGKGKAIIAMPSVAKKKDGSMISKIVPFIDHGAAVTTSRNDADYVVTEYGIAEMKGKSLQDRARALINIAHPDFKDELKAEFEKRFNAAF

SEQ ID NO: 209 - protein, 433 aa, synthetic construct
  REGION 1..433: note = Description of Artificial Sequence: Synthetic polypeptide
MDWKKLYKSKLVSAKEAVSNIKSNSRVVVSIAAAEPTELIDALVANKENYENVEVVQMVDMGKSEYAQEGMEKYFKYNSIFVGASTKDAVNSGRSDFTPCCFYELPRLFEEGYLPVDVVLIQVSKPDKHGYCSFGVSNDYTKPAADCAKMVIAEVNENMPRVLGDSFIHISDIDYIVETSHPIMELKQPKIGKIEEAIGEYCASLIEDGSTLQLGIGAIPDAVLLFLKDKRDLGIHSEMISDGVVDLVESGVINNKEKTLNPGKIVVTFFMGTKKLYDFIDDNPMVESYPVSYVNDPTVIMKNSKMISINSCVEVDLMGQVCSESIGMNQISGIGGQVDFIRGANMCKDGKAIIAIPSTAAKGKVSRIVPLIEKGTPITTSRTDVDYIITEYGIARLKSKSLKERARALINIAHPDFRAWLIDEYEKRFKTKF

SEQ ID NO: 210 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 211 - protein, 389 aa, Clostridium tetani
MKRFTIPRDIYYGKGSLEVIKSIKGNKAVIVIGGGSMKRFGFLDKVENYLKEAGIETKLIEGVEPDPSVETVMKGAEIMREFKPDWIISIGGGSPIDAAKAMWLFYEHPDLTFEEAAQKVLCLPELRSKAKFMAIPSTSGTASEVTAFSVITDYKKQIKYPLADFNLTPDIAIVDPELAETMPAKLTAHTGMDALTHAIEAYVATENTSFTDPLALQAILMVKDYLIKSYEEDKEARERMHEAQCLAGMAFSNALLGITHSMAHKIGAVFDITHGCANAIFLPYVIQFNSKVCEERYANIAKYLGLEGESDKDLVNSLINFIRDLNKKLSIDNSLKEYGIDENEFKEKVEYMAHNAVLDACTGANPRKINEEEMKNMYTYAFNGKDIDF

SEQ ID NO: 212 - protein, 405 aa, Butyrivibrio proteoclasticus
MSRFTLPRDIYHGKGALEALKTLEGKRAIVCVGGSSMKKGGFLQKVEDYLKEAGMEVELFEGIEPDPSVETVMKGAEAMQKFQPDWIVAIGGGSPIDAAKAMWIKYEYPETTFEDMCKVFGLPKLRTKAHFCAIPSTSGTATEVTAFSIITDYQKGIKYPIADFEITPDVAIVDPELTHTMPIKLVAHTGMDAMTHAIEAYVSTANCDYTDGLAIHAIEMIQANLVKSYNGDMESRDAMHNAQCLAGMAFSNALLGIVHSMAHKTGAVFADKGAHIIHGAANAMYLPKVIAFNAKDETAKKRYGVIADYMHLGGSNDDEKVKLLIDYLRKMNDDLNIPHSINHYGADGLPADQGFVAEDVFLERLHDIAANAILDACTGSNPRQPSQEEMEKLLKCCYYDTEVDF

SEQ ID NO: 213 - protein, 391 aa, Anaerostipes caccae
MGRFTLPRDLYFGKGSLETLKTLDGKKAVLVLGGGSMKRFGFVDQVVDYLKEAGIETKLIEGVEPDPSVETVFKGAEVMRDFEPDWIIAMGGGSPIDAAKAMWIFYEHPETTFDDVKDPFTVPPLRNKAKFLAIPSTSGTATEVTAFAVITDYSSGIKYPLADFEITPDIAIVDPALAETMPPKLTSYTGMDALTHAIEAYVAGLHSPFSDPLAMEAIEMVFDNLKASYEGDMTARENMHYAQCLAGMSFSNALLGIVHSLAHKTGAVFDTGHITHGCANAIYLPYVIQFNAKVCEERYAAIARRIGLPGNSDKALTLSLIEAIREMNKKLNIVSNLKDFGINEEEFNQKKRMIAERAVADACTGSNPRETSVDDMEKVLECIYYGKDVTF

SEQ ID NO: 214 - protein, 388 aa, Clostridium beijerinckii
MARFTLPRDLYWGKGSLENLKTLQGKKAVLVLGGGSMKRFGFVDKAVSYLKEAGIETRLFENVEPDPSVETVMKGAEFMREFEPDWIIAMGGGSPIDAAKAMWVFYEYPECTFEEILTPFSFPELRKKAKFCAIPSTSGTATEVTAFSVITDYAKGIKYPLADFNITPDVAIVDPELAETMPKHLVAYTGMDALTHAIEAYVSTLHGTFTDPLALKAIQIVNDELTKSYDGDMASREEMHYGQCLAGMAFSNALLGIVHSMAHKTGAAFSTGHITHGLANAMYLPYVIKYNAKEAEAKKRYETIAAYLGLEASVDALCNRIVELNNYMGIPNTLKEFGIKEDEFKEKISEIAKNAVGDACTGSNPRSIDPETMEKLFTYIYDGKEVDF

SEQ ID NO: 215 - protein, 388 aa, Clostridium celatum
MARFTLPRDIYHGNGSLEVLKTLKGKKAVVVVGGGSMKRFGFLEKVENYLKEAGLEVRLIEGVEPDPSVETVMNGAAVMREFEPDWIVAMGGGSPIDAAKAMWIFYEYPDFTFEEAVVPFGLPELRQKAKFVAIPSTSGTATEVTAFSVITDYKAKIKYPLADFNITPDIAIVDPELAQTMPAKLVAHTGMDALTHAIEAYTASLKSNFSDPLALKAIEMINENLVKSFNGDKNARDLMHEAQCLAGMAFSNALLGIVHSMAHKTGAVFHIPHGCANAIFLPYVIQYNRSECEERYADIAKVLKLEGNTNAELTDSLITKINEFNKELNIPATMQEYGVNEEEFLANLKNVAHNAVLDACTGSNPRTIDDETMEKLLSCTFYGNKVDF

SEQ ID NO: 216 - protein, 388 aa, Clostridium carboxidivorans
MSRFTLPRDIYFGENTLETLKTLKGKKAIIVVGGGSMKKFGFLQKVEEYLKEAGMEIKLIEGVEPDPSVETVMKGAEIMRDFEPDWIVSIGGGSPIDAAKAMWIFYEYPEFTFEQAVVPFGIPDLRQKAKFVAIPSTSGTATEVTAFSVITDYKAKIKYPLADFNLTPDVAIIDPALAQTMPAKLTAHTGMDALTHAIEAYVAGLRSYFSDPLAMQAIVMTKDNLIKSYEGDKEARDEMHIAQCLAGMAFSNALLGITHSMAHKTGAVFHIPHGCANAIFLPYVIDFNKKTCKDRYATIAKTLGLAGNTDDELVDALTSMIQEMNKKMDIPLNLKEYGVTEEDFNENLDFIAHNAVLDACTGSNPRPITEEEMKKVFKCTFTGEKVNF

SEQ ID NO: 217 - protein, 388 aa, Clostridium butyricum
MARFTLPRDIYHGEGSLEVLKTLKGKKAFVVVGGGSMKRFGFLQKVENYLKEAGMEVELFEGVEPDPSVETVMKGAEAMRKFQPDWIVAMGGGSPIDAAKAMWIFYEYPDFTFEQAVVPFGLPELRQKAKFVAIPSTSGTATEVTAFSVITNYTEKIKYPLADFNITPDIAIVDPELAQTMPAKLVAHTGMDALTHAIEAYTASLRSNFSDPLAIKAIEMVNENLVKSFEGDKTARNLMHEAQCLAGMAFSNALLGIVHSMAHKVGAVFHIPHGCANAIFLPYVIKYNRKVCEDRYADIARALKLEGNTDAELTDSLIKLINEFNDDLSIPHSMKEYGVEEADFKSNVKFIAHNAILDACTGSSPREIDEETMEKLFECTYYGTDVNF

SEQ ID NO: 218 - protein, 388 aa, Clostridium saccharoperbutylacetonicum
MARFTLPRDLYHGEGALEVLKTLKGKKAFVVVGGGSMKRFGFLQKVENYLKEAGMEVELFEGVEPDPSVETVMKGAEAMRNFEPDWIVSIGGGSPIDAAKAMWIFYEYPDFTFEQAVVPFGLPELRQKAKFVAIPSTSGTATEVTAFSVITNYTERIKYPLADFNITPDIAIIDPELAQTMPKTLTAHTGMDALTHAIEAYTASLRSNFSDPLAIKAIEMVNENLFKSYEGDKEARNLMHEAQCLAGMAFSNALLGIVHSMAHKVGAVFHIPHGCANAIFLPYVIQYNRTKCEERYADIARALKLEGNNDAELTDALIARINKLNEKLSIPSSMKEYGVTEEDFKANLSFIAHNAVLDACTGSNPREIDDATMEKLFECTFYGTKVEL

SEQ ID NO: 219 - protein, 388 aa, Clostridium beijerinckii
MARFTLPRDIYHGEGALEALKTLKGKKAFLVVGGGSMKRFGFLKQVEDYLKEAGMEVELFEGVEPDPSVETVMKGAEAMRNFEPDWIVAMGGGSPIDAAKAMWIFYEYPDFTFEQAVVPFGLPDLRQKAKFVAIPSTSGTATEVTAFSVITNYSEKIKYPLADFNITPDIAIVDPALAQTMPKTLTAHTGMDALTHAIEAYTASLQSNFSDPLAIKAVEMVQENLIKSFEGDKEARNLMHEAQCLAGMAFSNALLGIVHSMAHKVGAVFHIPHGCANAIFLPYVIEYNRTKCENRYGDIARALKLKGNNDAELTDSLIELINGLNDKLEIPHSMKEYGVTEEDFKANLSFIAHNAVLDACTGSNPREIDDATMEKLFECTYYGTKVNL

SEQ ID NO: 220 - protein, 393 aa, Clostridium beijerinckii
MSRFTLPRDIYYGANALEALKTLKGKKAVMVLGGGSMKRFGFVDKAIGYLKEAGIETKLFENVEPDPSVETVMKGAAMMREFEPDWIISMGGGSPIDAAKAMWAFYEYPETTFEDLITPFNFPELRQKAKFCAIPSTSGTATEVTAFSVITDYAKGIKYPLADFNITPDVAIVDPALAETMPPHLVAYTGMDALTHAIEAYVSTLHTVFTDPLAIEAIHIVEKDLIKSFKGDMTCREEMHYGQCLAGMAFSNALLGIVHSMAHKTGAAFSTGHITHGLANAMYLPYVIAYNSKADGVAERYADIARTMGVVGDTTAELVEGLRAKIRSMNDAMGIPNTLKDFGIIEDEFKEKLEAIATNAVGDACTGSNPRPIDPATMAKLFTCIYYGTEVDF

SEQ ID NO: 221 - protein, 394 aa, Clostridium perfringens
MGGIISMARFTLPRDIYHGKDSLEVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIVPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSFKGDMEARGEMHEAQCLAGMAFSNALLGIVHSMAHKVGAVFHIPHGCANAIFLPYVIKYNRKACEDRYAQIARHIGLKGESERELTDALIDLINKFNKELNIPSSMKEYGIDENEFKTNLKFIAHNAVLDPCTGSNPREIDDETMEKLYTCAYYGSDVDF

SEQ ID NO: 222 - protein, 858 aa, synthetic construct
  REGION 1..858: note = Description of Artificial Sequence: Synthetic polypeptide
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVDIPTTAGTGSEATPFAVITNDETGMKYGLTSYELTPNMAIIDTELMLNMPRKLTAAVGIDAFVHAIETYVSVMATDYTDELALRAIKMIFKYLPRAYKNGTNDIEAREKMAHASNIAGMAFANAFLGVCHSMAHQLGPMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQSTTANPRYPLISELKDIYIKSF

SEQ ID NO: 223 - protein, 389 aa, synthetic construct
  REGION 1..389: note = Description of Artificial Sequence: Synthetic polypeptide
MKRFTIPRDIYYGKGSLEVIKSIKGNKAVIVIGHASMKRFGFLDKVENYLKEAGIETKLIEGVEPDPSVETVMKGAEIMREFKPDWIISIGGGSPIDAAKAMWLFYEHPDLTFEEAAQKVLCLPELRHKAKFMDIPSTSGTASEVTAFSVITDYKKQIKYGLADFNLTPDIAIVDPELAETMPAKLTAHVGMDALTHAIETYVATERTSFTDPLALQAILMVHDYLIKSYEEDKEARERMHEAQCLAGMAFSNALLGITHSMAHQIGPVFDITHGCATAIFLPYVIQFNSKVCEERYANIAKYLGLEGESDKDLVNSLINFIRDLNKKLSIDNSLKEYGIDENEFKEKVEYMAHNAVLDACTGANPRKINEEEMKNMYTYAFNGKDIDF

SEQ ID NO: 224 - protein, 405 aa, synthetic construct
  REGION 1..405: note = Description of Artificial Sequence: Synthetic polypeptide
MSRFTLPRDIYHGKGALEALKTLEGKRAIVCVGHASMKKGGFLQKVEDYLKEAGMEVELFEGIEPDPSVETVMKGAEAMQKFQPDWIVAIGGGSPIDAAKAMWIKYEYPETTFEDMCKVFGLPKLRHKAHFCDIPSTSGTATEVTAFSIITDYQKGIKYGIADFEITPDVAIVDPELTHTMPIKLVAHVGMDAFTHAIETYVSTARCDYTDGLAIHAIEMIHANLVKSYNGDMESRDAMHNAQCLAGMAFSNALLGIVHSMAHQTGPVFADKGAHIIHGAATAMYLPKVIAFNAKDETAKKRYGVIADYMHLGGSNDDEKVKLLIDYLRKMNDDLNIPHSLNHYGADGLPADQGFVAEDVFLERLHDIAANAILDACTGSNPRQPSQEEMEKLLKCCYYDTEVDF

SEQ ID NO: 225 - protein, 391 aa, synthetic construct
  REGION 1..391: note = Description of Artificial Sequence: Synthetic polypeptide
MGRFTLPRDLYFGKGSLETLKTLDGKKAVLVLGHASMKRFGFVDQVVDYLKEAGIETKLIEGVEPDPSVETVFKGAEVMRDFEPDWIIAMGGGSPIDAAKAMWIFYEHPETTFDDVKDPFTVPPLRHKAKFLDIPSTSGTATEVTAFAVITDYSSGIKYGLADFEITPDIAIVDPALAETMPPKLTSYVGMDAFTHAIETYVAGLRSPFSDPLAMEAIEMVHDNLKASYEGDMTARENMHYAQCLAGMSFSNALLGIVHSLAHQTGPVFDTGHITHGCATAIYLPYVIQFNAKVCEERYAAIARRIGLPGNSDKALTLSLIEAIREMNKKLNIVSNLKDFGINEEEFNQKKRMIAERAVADASTGSNPRETSVDDMEKVLECIYYGKDVTF

SEQ ID NO: 226 - protein, 388 aa, synthetic construct
  REGION 1..388: note = Description of Artificial Sequence: Synthetic polypeptide
MARFTLPRDLYWGKGSLENLKTLQGKKAVLVLGHASMKRFGFVDKAVSYLKEAGIETRLFENVEPDPSVETVMKGAEFMREFEPDWIIAMGGGSPIDAAKAMWVFYEYPECTFEEILTPFSFPELRHKAKFCDIPSTSGTATEVTAFSVITDYAKGIKYGLADFNITPDVAIVDPELAETMPKHLVAYVGMDALTHAIETYVSTLRGTFTDPLALKAIQIVHDELTKSYDGDMASREEMHYGQCLAGMAFSNALLGIVHSMAHQTGPAFSTGHITHGLANAMYLPYVIKYNAKEAEAKKRYETIAAYLGLEASVDALCNRIVELNNYMGIPNTLKEFGIKEDEFKEKISEIAKNAVGDASTGSNPRSIDPETMEKLFTYIYDGKEVDF

SEQ ID NO: 227 - protein, 388 aa, synthetic construct
  REGION 1..388: note = Description of Artificial Sequence: Synthetic polypeptide
MARFTLPRDIYHGNGSLEVLKTLKGKKAVVVVGHASMKRFGFLEKVENYLKEAGLEVRLIEGVEPDPSVETVMNGAAVMREFEPDWIVAMGGGSPIDAAKAMWIFYEYPDFTFEEAVVPFGLPELRHKAKFVDIPSTSGTATEVTAFSVITDYKAKIKYGLADFNITPDIAIVDPELAQTMPAKLVAHVGMDAFTHAIETYTASLRSNFSDPLALKAIEMIHENLVKSFNGDKNARDLMHEAQCLAGMAFSNALLGIVHSMAHQTGPVFHIPHGCATAIFLPYVIQYNRSECEERYADIAKVLKLEGNTNAELTDSLITKINEFNKELNIPATLQEYGVNEEEFLANLKNVAHNAVLDASTGSNPRTIDDETMEKLLSCTFYGNKVDF

SEQ ID NO: 228 - protein, 388 aa, synthetic construct
  REGION 1..388: note = Description of Artificial Sequence: Synthetic polypeptide
MSRFTLPRDIYFGENTLETLKTLKGKKAIIVVGHASMKKFGFLQKVEEYLKEAGMEIKLIEGVEPDPSVETVMKGAEIMRDFEPDWIVSIGGGSPIDAAKAMWIFYEYPEFTFEQAVVPFGIPDLRHKAKFVDIPSTSGTATEVTAFSVITDYKAKIKYGLADFNLTPDVAIIDPALAQTMPAKLTAHVGMDAFTHAIETYVAGLRSYFSDPLAMQAIVMTHDNLIKSYEGDKEARDEMHIAQCLAGMAFSNALLGITHSMAHQTGPVFHIPHGCATAIFLPYVIDFNKKTCKDRYATIAKTLGLAGNTDDELVDALTSMIQEMNKKMDIPLNLKEYGVTEEDFNENLDFIAHNAVLDASTGSNPRPITEEEMKKVFKCTFTGEKVNF

SEQ ID NO: 229 - protein, 388 aa, synthetic construct
  REGION 1..388: note = Description of Artificial Sequence: Synthetic polypeptide
MARFTLPRDIYHGEGSLEVLKTLKGKKAFVVVGHASMKRFGFLQKVENYLKEAGMEVELFEGVEPDPSVETVMKGAEAMRKFQPDWIVAMGGGSPIDAAKAMWIFYEYPDFTFEQAVVPFGLPELRHKAKFVDIPSTSGTATEVTAFSVITNYTEKIKYGLADFNITPDIAIVDPELAQTMPAKLVAHVGMDAFTHAIETYTASLRSNFSDPLAIKAIEMVHENLVKSFEGDKTARNLMHEAQCLAGMAFSNALLGIVHSMAHQVGPVFHIPHGCATAIFLPYVIKYNRKVCEDRYADIARALKLEGNTDAELTDSLIKLINEFNDDLSIPHSLKEYGVEEADFKSNVKFIAHNAILDASTGSSPREIDEETMEKLFECTYYGTDVNF

SEQ ID NO: 230 - protein, 388 aa, synthetic construct
  REGION 1..388: note = Description of Artificial Sequence: Synthetic polypeptide
MARFTLPRDLYHGEGALEVLKTLKGKKAFVVVGHASMKRFGFLQKVENYLKEAGMEVELFEGVEPDPSVETVMKGAEAMRNFEPDWIVSIGGGSPIDAAKAMWIFYEYPDFTFEQAVVPFGLPELRHKAKFVDIPSTSGTATEVTAFSVITNYTERIKYGLADFNITPDIAIIDPELAQTMPKTLTAHVGMDAFTHAIETYTASLRSNFSDPLAIKAIEMVHENLFKSYEGDKEARNLMHEAQCLAGMAFSNALLGIVHSMAHQVGPVFHIPHGCATAIFLPYVIQYNRTKCEERYADIARALKLEGNNDAELTDALIARINKLNEKLSIPSSLKEYGVTEEDFKANLSFIAHNAVLDASTGSNPREIDDATMEKLFECTFYGTKVEL

SEQ ID NO: 231 - protein, 388 aa, synthetic construct
  REGION 1..388: note = Description of Artificial Sequence: Synthetic polypeptide
MARFTLPRDIYHGEGALEALKTLKGKKAFLVVGHASMKRFGFLKQVEDYLKEAGMEVELFEGVEPDPSVETVMKGAEAMRNFEPDWIVAMGGGSPIDAAKAMWIFYEYPDFTFEQAVVPFGLPDLRHKAKFVDIPSTSGTATEVTAFSVITNYSEKIKYGLADFNITPDIAIVDPALAQTMPKTLTAHVGMDAFTHAIETYTASLRSNFSDPLAIKAVEMVHENLIKSFEGDKEARNLMHEAQCLAGMAFSNALLGIVHSMAHQVGPVFHIPHGCATAIFLPYVIEYNRTKCENRYGDIARALKLKGNNDAELTDSLIELINGLNDKLEIPHSLKEYGVTEEDFKANLSFIAHNAVLDASTGSNPREIDDATMEKLFECTYYGTKVNL

SEQ ID NO: 232 - protein, 393 aa, synthetic construct
  REGION 1..393: note = Description of Artificial Sequence: Synthetic polypeptide
MSRFTLPRDIYYGANALEALKTLKGKKAVMVLGHASMKRFGFVDKAIGYLKEAGIETKLFENVEPDPSVETVMKGAAMMREFEPDWIISMGGGSPIDAAKAMWAFYEYPETTFEDLITPFNFPELRHKAKFCDIPSTSGTATEVTAFSVITDYAKGIKYGLADFNITPDVAIVDPALAETMPPHLVAYVGMDAFTHAIETYVSTLRTVFTDPLAIEAIHIVHKDLIKSFKGDMTCREEMHYGQCLAGMAFSNALLGIVHSMAHQTGPAFSTGHITHGLATAMYLPYVIAYNSKADGVAERYADIARTMGVVGDTTAELVEGLRAKIRSMNDAMGIPNTLKDFGIIEDEFKEKLEAIATNAVGDASTGSNPRPIDPATMAKLFTCIYYGTEVDF

SEQ ID NO: 233 - protein, 394 aa, synthetic construct
  REGION 1..394: note = Description of Artificial Sequence: Synthetic polypeptide
MGGIISMARFTLPRDIYHGKDSLEVLKSLEGKKAFIVIGHASMKRFGFLDKVLSYLKEANMETKVFEGVEPDPSVETVMKGAKEMEEFNPDWIVSIGGGSPIDAAKAMWIFYEYPDFTFEKAIVPFGLPKLRHKAKFVDIPSTSGTATEVTAFSVITDYKAKIKYGLADFEITPDIAIVDPSLAETMPEKLVAHVGMDAFTHAIETYTASLRSNFTDPLALKAIEMVHMHLVNSFKGDMEARGEMHEAQCLAGMAFSNALLGIVHSMAHQVGPVFHIPHGCATAIFLPYVIKYNRKACEDRYAQIARHIGLKGESERELTDALIDLINKFNKELNIPSSMKEYGIDENEFKTNLKFIAHNAVLDPSTGSNPREIDDETMEKLYTCAYYGSDVDF

SEQ ID NO: 234 - protein, 468 aa, Clostridium beijerinckii; identical to SEQ ID NO: 70

SEQ ID NO: 235 - protein, 467 aa, Clostridium botulinum
MERNLSVLSQTNDLKITKRTEGDKSNNKESYLGVFKKVENAITKAIYAQKKLSLYYTKEDRERIIKSIRKATLENKEILAKMIVDETHMGRYEDKILKHELVAKYTPGTEDLITTAWSGDQGLTLVEMSPYGVIGAITPSTNPTETVICNSIGMIAAGDSVVFNGHPGAKKCVAFAVDMINKAVIREGGPENLVTTVENPTMESLNVIMKHPYIKLLCGTGGPGLIKTLLNSGKKAIGAGAGNPPVIVDDSADIDKAAKNIIEGCSFDNNLPCIAEKEVFVFENVANDLIQNMIKNNAVLINENQVSKLLDLVLLERKDETLEYAINKKWVGKDAKLFLDKIGIKASDNVRCIICEVDANHPFVMTELMMPILPIVRVKDVDEAIECAKTAEQRKRHSAYMYSKNIDNLNRFEKEIDTTIFVKNAKSFAGVGFGAEGFTTFTIAGPTGEGITSARNFTRQRRCVLAG

SEQ ID NO: 236 - protein, 468 aa, Clostridium beijerinckii; identical to SEQ ID NO: 70

SEQ ID NO: 237 - protein, 468 aa, Clostridium saccharoperbutylacetonicum
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSASHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 238 - protein, 480 aa, Pelosinus fermentans
MSIDQALIEKITLEILTKMQTGAKAAPAGYGDGIFETVDEAVAAARKAYQELKTLSLEKREVLIKAMRDVAYENATILAQMAVDESGMGRVSDKIIKNQVAALKTPGTEDLTTQAWSGDNGLTLIEMGPYGVIGAITPTTNPTETVICNGIGMIAAGNTVFFSPHPTAKNTSMKIITLLNQAIVKAGGPNNLLTSVANPSIKAANEMMKHPGINMLVATGGPGVVKAVLSSGKKAIGAGAGNPPVIVDETADIEKAARDIVAGCSFDNNLPCIAEKEVIAIGSIADRLITYMQKYGAYLISGSNIDRLLNVIMTVQEEKIAEGCTDKPKRSYGINKDYVGKDAKYLLSKIGIDVPDSVRVVLCETPADHPFVIEELMMPVLPVVQVKDIDEAIEVAVRVEHGNRHTAAMHSKNVDHLTRFARAVETTIFVKNAPSYAGIGVGGEGFTSFTLAGPTGEGITSPRSFTRQRRCVLVDAFSIV

SEQ ID NO: 239 - protein, 468 aa, Clostridium beijerinckii
MNKDTLIPTTKDLKLKTNVENINLKNYKDNSSCFGVFENVENAINSAVHAQKILSLHYTKEQREKIITEIRKAALENKEVLATMILEETHMGRYEDKILKHELVAKYTPGTEDLTTTAWSGDNGLTVVEMSPYGVIGAITPSTNPTETVICNSIGMIAAGNAVVFNGHPGAKKCVAFAIEMINKAIISCGGPENLVTTIKNPTMESLDAIIKHPLIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYFINKKWVGKDAKLFSDEIDVESPSNIKCIVCEVNANHPFVMTELMMPILPIVRVKDIDEAVKYTKIAEQNRKHSAYIYSKNIDNLNRFEREIDTTIFVKNAKSFAGVGYEAEGFTTFTIAGSTGEGITSARNFTRQRRCVLAG

SEQ ID NO: 240 - protein, 527 aa, Caldalkalibacillus thermarum
MNMTEKDIEKIVQSVLHNVESALGKSASASPSVSAVSVASGEGIKPVQFKQVPVFQQETVKSPNRNRNLGGAEEKWGVFNHMEDAIEASYRAQMEFVKHFQLKDREKIITAIREAVLREKEVLARKVYEETKIGRYEDKVAKHELAALKTPGTEDLKTEAFSGDNGLTIVERAPYGLIGAVTPVTNPTETIINNAIGMLAAGNAVVFNVHPSSKRSCAYAVQLINKAITEAGGPHHLVTMVKEPTLDTLQTLIDSPKVKLLVGTGGPGLVQTLLKSGKKAIGAGAGNPPVIVDDTADLEHAARSIIEGAAFDNNLLCIAEKEVFVLESVADDLIFHMLNHGAYMLGQHEVEQVMAFALEEQGNEQNRGCGFNPQRHYQVSKDWIGQDARLFLEHIGVQPPTEVKLLICDVEFDHPFVQLEQMMPVLPIVRVKTLDEAIEKAVMAEHGNRHTAIMHSKNVDHLTKFARAIQTTLFVKNASSLAGVGYGGEGHTTMTIAGPTGEGVTSAKTFTRERRCVLAEGGFRIIG

SEQ ID NO: 241 - protein, 468 aa, synthetic construct
  REGION 1..468: note = Description of Artificial Sequence: Synthetic polypeptide
MNKDTLIPTTKLLKVKTNGENINLKNYKDNSSCKGVFENVEDALSSTVHARKILSGACGKEQREKIFSEIAKAALQNKEVLATMILEETHRGRYEDKILKHELRATVTPGTENLTTTAWSGDNGLTINAMWAMGPAGASTPLTNPTETVICNSIGCIVGGNAVAFNPHPCAGKCVAYGVEMINMAIKGCGGPLNLVTTIMNPTLESLAAICKHPMIQLLCGTGGPRLVAVILQGGKKAIGSSRGNPPTIVDSFADIERTSRSIIISCSLDNNIPCIAETSLFVPENVLKDVISVFLKLNAVIINEDQVSLLIDLVLAKNNETQEYFKNKKWVAKDAKLFLDEIDVESPWNVKNIACEVNENHPFVMTELLMPILPIVRVKDIYEAIKYAKIAEQPHKFSAMIYSKHIDNLNRFEREIDTTIFVKNAKSFAGVGYEAEGFTTFTIAGSTGEGITSARNFTRQRRCVLAG

SEQ ID NO: 242 - protein, 465 aa, synthetic construct
  REGION 1..465: note = Description of Artificial Sequence: Synthetic polypeptide
MERNLSVLSQTNLKPTKRTEGDKSNNKESYKGVFKKVEDALTKTIYARKKLSGACGKEDRERFSKSIAKATLENKEILAKMIVDETHRGRYEDKILKHELRATVTPGTENLITTAWSGDQGLTINAMWAMGPAGASTPLTNPTETVICNSIGCIVGGDSVAFNPHPGAGKCVAYGVDMINMAVKSEGGPLNLVTTVMNPTLESLAVICKHPMIQLLCGTGGPRLIAVILQGKKAIGSSRGNPPTIVDSFADIDRTSKNIIISCSLDNNIPCIAETSLFVPENVLNKVIQVFIKLNAVLINENQVSLLLDLVLLARKDETLEYAKNKKWVAKDAKLFLDKIGIKASWNVRNIACEVDENHPFVMTELLMPILPIVRVKDVYEAIECAKTAEQPKRFSAMMYSKHIDNLNRFEKEIDTTIFVKNAKSFAGVGFGAEGFTTFTIAGPTGEGITSARNFTRQRRCVLAG

SEQ ID NO: 243 - protein, 468 aa, synthetic construct; identical to SEQ ID NO: 241
  REGION 1..468: note = Description of Artificial Sequence: Synthetic polypeptide

SEQ ID NO: 244 - protein, 468 aa, synthetic construct
  REGION 1..468: note = Description of Artificial Sequence: Synthetic polypeptide
MIKDTLVSITKLLKPKTNVENANLKNYKDNSSCKGVFENVEDALSNTVHARKILSGACGKEQREKFSTEIAKAALENKEILATMILEETHRGRYEDKILKHELRATVTPGTENLTTTAWSGDNGLTINAMWAMGPAGASTPLTNPTETVICNSIGCIVGGNTVAFNPHPGAGKCVAYGVEMINMAIKGCGGPLNLVTTIMNPTLDSLAAICKHPMIQLLCGTGGPRLVAVILQGGKKAIGSSRGNPPTIVDSFADIERTGKSIIISCSLDNNIPCIAETSLFVPENVLKDVISVFLKLNAVIINEDQVSLLIDLVLAKNNETQEYSKNKKWVAKDAKLFLDEIDVESPWSVKCIACEVSESHPFVMTELLMPILPIVRVKDIYEAIEYAKIAERPHKFSAMIYSKHIDNLWRFERGADSTIFVKNGLSLAGVGYEAPGFTSFTIAVPTGEGMTSAANFTRQRRTVNAN

SEQ ID NO: 245 - protein, 480 aa, synthetic construct
  REGION 1..480: note = Description of Artificial Sequence: Synthetic polypeptide
MSIDQALIEKITLEILTKMQTGAKAAPAGYGDGIFETVDDALAATRKARQELKGCGLEKREVFSKAMADVAYENATILAQMAVDESGRGRVSDKIIKNQVRATVTPGTENLTTQAWSGDNGLTINAMWAMGPAGASTPLTNPTETVICNGIGCIVGGNTAFFSPHPTAGNTSMYGITLLNMAIKGAGGPLNLLTSVMNPSLKAAAEMCKHPMIQMLVATGGPRLVAVILQGGKKAIGSSRGNPPTIVSEFADIERTSRDIVISCSLDNNIPCIAETSLIAPGSILKRVITVFQKLGAYLISGSNIDRLLNVIMAVQEEKIAEGCTDKPKRSYGKNKDYVAKDAKYLLSKIGIDVPDWVRNVACETPEDHPFVIEELHMPVLPVVQVKYIDEAIEVAVRVEHPHRFTAMMHSKHVDHLTRFARAVETTIFVKNAPSYAGIGVGGEGFTSFTLAGPTGEGITSPRSFTRQRRCVLVDAFSIV

SEQ ID NO: 246 - protein, 468 aa, synthetic construct
  REGION 1..468: note = Description of Artificial Sequence: Synthetic polypeptide
MNKDTLIPTTKLLKPKTNVENINLKNYKDNSSCKGVFENVEDALNSTVHARKILSGACGKEQREKFSTEIAKAALENKEVLATMILEETHRGRYEDKILKHELRATVTPGTENLTTTAWSGDNGLTINAMWAMGPAGASTPLTNPTETVICNSIGCIVGGNAVAFNPHPGAGKCVAYGIEMINMAIKGCGGPLNLVTTIMNPTLESLAAICKHPMIQLLCGTGGPRLVAVILQGGKKAIGSSRGNPPTIVDSFADIERTGKSIIISCSLDNNIPCIAETSLFVPENVLKDVISVFLKLNAVIINEDQVSLLIDLVLAKNNETQEYFKNKKWVAKDAKLFSDEIDVESPWNIKCVACEVNENHPFVMTELLMPILPIVRVKDIYEAVKYTKIAERPHKFSAMIYSKHIDNLWRFERGADSTIFVKNGLSLAGVGYEAPGFTSFTIAVPTGEGMTSAANFTRQRRTVNAN

SEQ ID NO: 247 - protein, 527 aa, synthetic construct
  REGION 1..527: note = Description of Artificial Sequence: Synthetic polypeptide
MNMTEKDIEKIVQSVLHNVESALGKSASASPSVSAVSVASGEGIKPVQFKQVPVFQQETVKSPNRNRNLGGAEEKWGVFNHMEDAIEASYRAQMEFVKHFQLKDREKIITAIREAVLREKEVLARKVYEETKRGRYEDKVAKHELRATVTPGTENLKTEAFSGDNGLTINARWAMGPAGASTPLTNPTETIINNAIGCLVGGNAVAFNPHPSSGRSCAYGVQLINMAIKGAGGPLHLVTMVMEPTRDTLATLCDSPMVQLLVGTGGPRIVAVILQGGKKAIGSSRGNPPTIVDSFADLERTSRSIISGAALDNNILCIAETSLFVPESVLKDVIFVFLNLGAYMLGQHEVELVMAFALAEQGNEQNRGCGFNPQRHYQKSKDWIAQDARLFLEHIGVQPPWEVKLNICDVEEDHPFVQLEQMMPVLPIVRVKTLDEAIEKAVMAEHGNRHTAIMHSKNVDHLTKFARAIQTTLFVKNASSLAGVGYGGEGHTTMTIAGPTGEGVTSAKTFTRERRCVLAEGGFRIIG

SEQ ID NO: 248 - protein, 434 aa, Fusobacterium sp.
MKNWKESYKAKICTPDEAIQKIKDAKRISFGHICSESTVLTEALLRNKKLFKKLEIAHLLSVGKSEYAKEENSEYFRHNALFIGPKTREAANSSYGDYTPTFFFETAKLFGKDGELSLDAMLLQVSPPDEHGYCSYGLSCDYTKSATESAKIVIAQINKFVPRTFGNCFVHIDDIDYIIEEDTPIPEVQPPVVGEIERKIGEFCASLINDGDTLQLGIGAIPVAVLNFLKDKKDLGIHSEMISDGIVDLINLGVITNKKKNLNPNKSIATFLMGSKKLYDYADNNPAIELHPVDYVNNPIIIAQNDNMISINSAIEVDLTGQVNAEYINSKEFSGPGGQVDFVRGATMSKGGKSIIALPSTAANGTISKIVFNFEEGVPVTTTRNDVDYVITEYGIAHLRGKTLRERAKLLIEIAHPNFREELRKKALEKFGKL

SEQ ID NO: 249 - protein, 434 aa, Fusobacterium sp.
MKNWKESYKAKICTPDEAIQKIKDAKRISFGHICSESTVLTEALLRNKKLFKKLEIAHLLSVGKSEYAKEENSEYFRHNALFIGPKTREAANSSYGDYTPTFFFETAKLFGKDGELSLDAMLLQVSPPDEHGYCSYGLSCDYTKSATESAKIVIAQINKFVPRTFGNCFVHIDDIDYIIEEDTPIPEVQPPVVGEIERKIGEFCASLINDGDTLQLGIGAIPVAVLNFLKDKKDLGIHSEMISDGIVDLINLGVITNKKKNLNPNKSIATFLMGSKKLYDYADNNPAIELHPVDYVNNPIIIAQNDNMVSINSAIEVDLTGQVNAEYINSKEFSGPGGQVDFVRGATMSKGGKSIIALPSTTANGTISKIVFNFEEGVPVTTTRNDVDYVITEYGIAHLRGKTLRERAKLLIEIAHPNFREELRKKALEKFGEL

SEQ ID NO: 250 - protein, 434 aa, Fusobacterium sp.
MKNWKESYKAKICTPDEAIQKIKDAKRISFGHICSESTVLTEALLRNKKLFKKLEIAHLLSVGKSEYAKEENSEYFRHNALFIGPKTREAANSSYGDYTPTFFFETAKLFGKDGELSLDAMLLQVSPPDEHGYCSYGLSCDYTKSATESAKIVIAQINKFVPRTFGNCFVHIDDIDYIIEEDTPIPEVQPPVVGEIERKIGEFCASLINDGDTLQLGIGAIPVAVLNFLKDKRDLGIHSEMISDGIVDLINLGVITNKKKNLNPNKSIATFLMGSKKLYDYADNNPAIELHPVDYVNNPIIIAQNDNMISINSAIEVDLTGQVNAEYINSKEFSGPGGQVDFVRGATMSKGGKSIIALPSTAANGTISKIVFNFEEGVPVTTTRNDVDYVITEYGIAHLRGKTLRERAKLLIEIAHPNFREELRKKALEKFGEL

SEQ ID NO: 251 - protein, 434 aa, Fusobacterium sp.
MKNWKKSYKAKICTPDEAIQKIKDAKRISFGHICSESTVLTEALLRNKKLFKKLEIAHLLSVGKSEYAKEENSEYFRHNALFIGPKTREAANSSYGDYTPTFFFETAKLFGKDGELSLDAMLLQVSPPDEHGYCSYGLSCDYTKSATESAKIVIAQINKFVPRTFGNCFVHIDDIDYIIEEDTPIPEVQPPVVGEIERKIGEFCASLINDGDTLQLGIGAIPVAVLNFLKDKRDLGIHSEMISDGIVDLINLGVITNKKKNLNPNKSIATFLMGSKKLYDYADNNPAIELHPVDYVNNPIIIAQNDNMISINSAIEVDLTGQVNAEYINSKEFSGPGGQVDFVRGATMSKGGKSIIALPSTAANGTISKIVFNFEEGVPVTTTRNDVDYVITEYGIAHLRGKTLRERAKLLIEIAHPNFREKLRKKALEKFGKL

SEQ ID NO: 252 - protein, 450 aa, Desulfomonile tiedjei
MNWKSYYNQRTFPAREAVKAIKSGDRVVHAHACGEPACLVNAMVDRADELRDVEIVHMVSMGKAKYCQPEYATSFRHNALFVGNTSRQAVNEGRGDYTCCYFSEIPLLFRNGLLPVDVALIAVSPPDKLGFVCLGISVDYTKQAALSAKTVIAAVNPNMPRIGGDSYLHVTDIDYFVPTEEPLIELQPPALGDLERAIGSNVASLIKDGDCLQLGIGALPDAVLSFLGEKTDLGIHSEMISDGVMKLVEAGVVTCKRKNFRPRKAVITFAMGTRGFYEWLNDNALMESYPVDFTNDPSIIAQNDNMVSINSAISVDLQGQVAADTLGSKQFSGVGGQVDFVRGANRSKGGRSIIAMPSTAAKGKASRIVAALDRGQAVTTPRNDVAYVVTENGVAALKGKTIRQRAEALIAIAAPEFQDRLREEFFEVYGLRGPTIQVSVQVAAGSAGAA

SEQ ID NO: 253 - protein, 428 aa, Porphyromonas uenonis
MSIPFSSITPDEAVSHIQDGDFVVLSHAAAVPQACVAALARNYERFHNVRIFHMVTLGESPYTAPEMEGHFRLVTNFVGANTRPAVDEQRADFVPSYFYEVPSMMQPGGAFHPDVAIVQLSYPNEEGYCSFGTSCDYTKPAAEQARVVIGELNKQMPFVGGDNLIHISQLTHIVEADYPLYALDLPRIGEVEEAIGRNCAELIQDGDTLQLGIGAIPDAVLLFLKDKKDLGIHSEMVSDGVVKLIKANTITGRRKTLHPNKVVATFLMGTQELYDFAHNNPTIEMRPVNYVNDPRIIAQNDQLVSINSCIEVDLMGQVVSEAMGLRQFSGPGGQVDFVRGAAWSKGGRSIIAIPSTARRGTASRIVPLLTEGAAITTSRNDVDYIVTEYGVAHLKGKSLRERALALTAIAHPNFRPELEAECKRRFKL

SEQ ID NO: 254 - protein, 431 aa, Pelosinus fermentans
MNNWRRVYQNKIVSAEQAVQHIKSQDRVVTGHACGEPQALIEALVARSPEIKDVEIVHMVAMGPAKYAQPGQEKSFRHNALFVGGTTRKAVEEKRADFTPCFFSEIPRLFKNNILPVDVALIQITPPDEQGFCSYGVSADYTKTAAECAKIVIAQVNHCLPRTGGASIHLDAVHFIVEQEEPLIELKPPKIGDIEKAIGEHVAALIPDGATLQLGIGAIPDAVLLFLTDKKDLGIHSEMFSDGVVVLAEAGVITNRKKTIHTGKFMATFLMGTKRLYDFVNNNPDVELQSVDYINDPYIIGQHENMISINSALQVNLMGEVNAEMIGPKQFSAVGGQVDFIRGASRSLQGKSIIALPSTASGGTISRIACELDRGAAVTTSRNDVHYVVTEYGMADLRGKCLGERAKSLIAISHPDFRSRLTKEAQEKGLM

SEQ ID NO: 255 - protein, 432 aa, Megasphaera sp.
MQDWKEKYAGKIVSPAEAVSHVKSGDKIVFSHACGESQVLTEALVKQADRLKDVEIIHMVAMGAAKYCLPEMEGHFRHNALFVGATTREAVESGRADYTPCFFHEAPRLFTDGTIPVDVVMLQVSEPDENGICSLGISVDYTQAAASVATLKIAQINKNMPYTYGNGIHLDDLDYLVIKDEPIIELPPPKIGEIEQRIGEHVASLIQDGDTLQLGIGAIPDAVLSFLGDKKDLGIHSEMFSDGVVDLAEKGVITNRKKSLDPGKFLSCFLMGTKKLYNFIDHNPDVIIRPVDYTNDPFVISQIDNIISINSAMQVDLMGQVNAEMIGLRQFSGVGGQVDFVRGAARAKGGKAIIAMPSTTGKTKKISKIVPLLDRGAAVTTSRNDVDYIVTEFGIAALHGKTLRQRAKALIAIAHPDFRASLEEEFNARFGL

SEQ ID NO: 256 - protein, 431 aa, Pelosinus fermentans
MNNWRRVYQNKIVSAEQAVQHIKSQDRVVTGHACGEPQALIEALVARSPEINNVEIVHMVAMGPAKYAQPGQEKSFRHNALFVGGTTRKAVEEKRADFTPCFFSEIPRLFKNNILPVDVALIQITPPDEQGFCSYGVSADYTKTAAECAKIVIAQVNHCLPRTGGASIHLDAVHFIVEQEEPLIELKPPKIGDIEKAIGEHVAALIPDGATLQLGIGAIPDAVLLFLTDKKDLGIHSEMFSDGVVVLAEAGVITNRKKTIHTGKFMATFLMGTKRLYDFVNNNPDVELQSVDYINDPYIIGQHENMISINSALQVNLMGEVNAEMIGPKQFSAVGGQVDFIRGASRSLQGKSIIALPSTASGGTISRIACELDRGAAVTTSRNDVHYVVTEYGMADLRGKCLGERAKSLIAISHPDFRSRLTKEAQEKGLM

SEQ ID NO: 257 - protein, 434 aa, Fusobacterium periodonticum
MKNWQEKYKTKICSPDEAIQKIKSAKRISFGHICSESTVLTEALVRNKQLFKKLEIDHLLSIGKCEYAKEENSEYFHHNALFIGPKTREAANSFYGDYTPIFFYQTAKIFGKDGDLAPDAMLLQVSPPDENGYCSYGLSCDYTKSATESAKIVIAQINKFVPRTLGNCFVHLDDIDYIILEDTPIPEIPAPVIGELEEKIGANCASLINDGDTLQLGIGAIPFAVLNFLKQKKDLGIHSEMVSDGIVDLIQAGVITNKKKNFNPNKVIATFLLGTKKLYDYADNNPAIELHPVDYVNNPMIIAQNDNMISINSAIQVDLMGQVNAEYINTKQFSGPGGQVDFVRGATMSNGGKSIIALPSTTADEKISKIVFTFEEGVPITTTRNDVDYIITEYGIAHLKGKTLRERAKLLIEIAHPKFREDLRKKAIEKFKIL

SEQ ID NO: 258 - protein, 434 aa, Fusobacterium nucleatum
MKNWKESYKAKICTPDEAIQKIKDAKRISFGHICSESTVLTEALLRNKKLFKKLEIAHLLSVGKSEYAKEENSEYFRHNALFIGPKTREAANSSYGDYTPTFFFETAKLFGKDGELSLDAMLLQVSPPDEHGYCSYGLSCDYTKSATESAKIVIAQINKFVPRTFGNCFVHIDDIDYIIEEDTPIPEVQPPVVGEIERKIGEFCASLINDEDTLQLGIGAIPVAVLNFLKDKKDLGIHSEMISDGIVDLINLGVITNKKKNLNPNKSIATFLMGSKKLYNYADNNPAIELHPVDYVNNPIIIAQNDNMISINSAIEVDLTGQVNAEYINSKEFSGPGGQVDFVRGATMSKGGKSIIALPSTAANGTISKIVFNFEEGVPVTTTRNDVDYVITEYGIAHLRGKTLRERAKLLIEIAHPNFREELREKALEKFGTL

SEQ ID NO: 259 - protein, 434 aa, Fusobacterium sp.
MKNWKESYKTKICTPDEAIQKIKDAKRISFGHICSESTVLTEALIRNKKLFKKLEIAHLLSVGKSEYAKEENSEYFRHNALFIGPNTREAANSSYGDYTPTFFFETAKLFGKAGELSLDAMLLQVSPPDEHGYCSYGLSCDYTKSATENAKIVIAQINKFVPKTFGNCFVHIDDIDYIIEEDTPIPEVQPLVVGEIERKIGEFCASLINDGDTLQLGIGAIPVAVLNFLKDKKDLGIHSEMISDGIVDLINLGVITNKKKNLNPNKTIATFLMGSKKLYDYANDNPAIELHPVDYVNNPIIIAQNDNMISINSAIEVDLTGQVNAEYINSKEFSGPGGQVDFVRGSTMSKGGKSIIALPSTAANGTISKIVFNFEEGVPVTTTRNDVDYIITEYGIAHLRGKTLRERARLLIEIAHPNFREELRKKALEKFGKL

SEQ ID NO: 260 - protein, 434 aa, Fusobacterium sp.
MEKWQKKYKAKICSPDEAIQKIKSAKRISFGHICSESSVLTEALIRNKKLFKKLEIDHLLSIGKCEYAKEENSEYFHHNALFIGPKTREAANSSYGDYTPIFFYETAKIFGKDGDLSPDAMLLQVSTPDEHGYCSYGLSCDHTKSATESAKIVVAQINKFVPRTLGNCFIHIDDIDYIILEDTPIPEIPAPVVGELEEKIGANCASLINDGDTLQLGIGAIPFAVLNFLKDKKDLGIHSEMISDGIVDLIQAGVITNKKKNFNPNKVIATFLLGTKKLYDYANNNPAIELHPVDYVNNPMIIAQNNNMISINSAIQVDLMGQVNAEYINTKQFSGPGGQVDFVRGATMSNGGKSIIALPSTTADEKISRIVFTFEEGVPVTTSRNDVDYIITEYGIAHLKGKTLRERAKLLIEIAHPKFREELRKRAIEKFEIL

SEQ ID NO: 261 - protein, 434 aa, Fusobacterium nucleatum
MKKWKESYKLKICTPDEAIQKIKDAKRISFGHICSESSVLTEALVRNKKLFKKLEIAHLLSVGKSEYAKEENSEYFRHNALFIGSKTREAANSSYGDYTPTFFFETAKLFGKDGELALDAMLLQVSTPDEHGYCSYGLSCDYTKSATENAKIVIAQINKFVPRTLGNCFVHIDDIDYIIEEDTPIPEVQPPVVGEIERKIGGFCASLVRDGDTLQLGIGAIPVAVLNFLKEKKDLGIHSEMISDGIVDLINLGVITNKKKNLNPNKAIATFLMGSKKLYDYANDNPAIELHPVDYVNNPIIIAQNDNMVSINSAIQVDLMGQVNAEYIDSKQFSGPGGQVDFVRGATMSKGGKSIIALPSTTAKESISRIVFAFDEGTPVTTSRNDVDYVITEYGIAHLRGKTLRERAKLLIEIAHPKFREELRKKALEKFGEL

SEQ ID NO: 262 - protein, 434 aa, Fusobacterium sp.
MEKWQEKYKAKICSPDEAIQKIKSAKRISFGHICSESSVLTEALIRNKKLFKKLEIDHLLSIGKCEYAKEENSEYFHHNALFIGSKTREAANSSYGDYTPIFFYETAKIFGKDGDLSPDAMLLQVSTPDEHGYCSYGLSCDYTKSATESAKIVVAQINKFVPRTLGNCFIHIDDIDYIILEDTPIPEIPTPVVGELEEKIGANCASLINDGDTLQLGIGAIPFAVLNFLKDKKDLGIHSEMVSDGIVDLIQAGIITNKRKNFNPNKVIATFLLGTKKLYDYANNNPAIELHPVDYVNNPMIIAQNNNMISINSAIQVDLMGQVNAEYINSKQFSGPGGQVDFVRGATMSNGGKSIIALPSTTADEKISRIVFTFEEGVPVTTSRNDVDYIITEYGIAHLKGKTLRERARLLIEIAHPKFREELRRKAIEKFEIL

SEQ ID NO: 263 - protein, 434 aa, Fusobacterium nucleatum
MKNWKESYKTKICTPDEAIQKIKDAKRISFGHICSESTVLTEALIRNKKLFKKLEIAHLLSVGKSEYAKEENSKYFRHNALFIGPKTREAANSSYGDYTPTFFFETAKLFGKDGELALDAMLLQVSTPDEHGYCSYGLSCDYTKSATENAKIVIAQINKFVPRTFGNCFVHIDDIDYIIEEDTPILEVQPPVVGETERKIGEFCASLVRDGDTLQLGIGAIPVAVLNFLKDKKDLGIHSEMISDGIVDLINLGVITNKKKNLNPNKAIATFLMGSKKLYDYADNNPAIELHPVDYVNNPIIIAQNDNMVSINSAIQIDLMGQVNAEYVDSKQFSGPGGQVDFVRGTTMSKGGKSIIALPSTTAKGTISRIVFTFDEGVPVTTSRNDVDYVITEYGIAHLRGKTLRERAKLLIEIAHPNFREELRKKALEKFGEL

SEQ ID NO: 264 - protein, 434 aa, Fusobacterium nucleatum
MKNWKESYKAKICTPDEAIQKIKDAKRISFGHICSESTVLTEALLRNKKLFKKLEIAHLLSVGKSEYAKEENSEYFRHNALFIGPKTREAANSSYGDYTPTFFFETAKLFGKDGELSLDAMLLQVSPPDEHGYCSYGLSCDYTKSATESAKIVIAQINKFVPRTFGNCFVHIDDIDYIIEEDTPIPEVQPPVVGEIERKIGEFCASLINDGDTLQLGIGAIPVAVLNFLKDKRDLGIHSEMISDGIVDLINLGVITNKKKNLNPNKSIATFLMGSKKLYDYADNNPAIELHPVDYVNNPIIIAQNDNMISINSAIEVDLTGQVNAEYINSKEFSGPGGQVDFVRGATMSKGGKSIIALPSTAANGTISKIVFNFEEGVPVTTTRNDVDYVITEYGIAHLRGKTLRERAKLLIEIAYPNFREELRKKALEKFGEL

SEQ ID NO: 265 - protein, 434 aa, Fusobacterium nucleatum
MKNWKESYKSKIGTPDEAIQKIKDAKRISFGHICSESTVLTEALVRNKKLFKKLEIAHLLSVGKSEYAKEENSEYFRHNALFIGPKTREAANSSYGDYTPTFFFETAKLFGKDGELALDAMLLQVSTPDEHGYCSYGLSCDYTKSATENAKIVIAQINKFVPRTLGNCFVHIDDIDYIIEEDTPIPEVQPPVVGEIERKIGEFCASLVRDGDTLQLGIGAIPVAVLNFLKDKKDLGIHSEMISDGIVDLINLGVITNKKKNLNPNKAIATFLMGSKKLYDYANDNPAIELHPVDYVNNPIIIAQNDNMVSINSAIQVDLMGQVNAEYVDSKQFSGPGGQVDFVRGATMSKGGKSIIALPSTTGKGTISRIVFTFDEGVPVTTSRNDVDYVITEYGIAHLRGKTLRERAKLLIEIAHPDFREELRKKALEKFGEL

SEQ ID NO: 266 - protein, 431 aa, Roseiflexus sp.
MPRTLTTGTREMTAEEAVELIESNHRVYIGGGCGVPLPLLQALVARASELRNVEIIHMLTAGEDPTTAPDLAASFRHNALFIGHNTRRAVNEGRADFTPIFLSEIPKLFRKGILPLDVAMIQVSPPDRHGFCSLGVEVGCTLPAARTARIVIAEVNARMPRTLGDSFIHVSRLTAMVESDRPLLELPQGETNGVAQAIGRHIAELIPDGATLQLGIGAIPDAVLSNLRGKRHLGIHTELFSDGVIDLVESGVIDGELKTIHQGKVVAGFILGSQRCFDWAHNNAMVEMHPTDYVNDPFVIAQHKNMVAVNSALQVDLTGQVCADSIGTRLYSGVGGQLDFIRGASRSEGGIPIIAISSTARDGTISRIVPTLDPGAGVVTTRYDVHFVVTEYGVADLYGRTLAQRARALINVAHPAFRDQLTEAAKKLHYI

SEQ ID NO: 267 - protein, 428 aa, Porphyromonas asaccharolytica
MSIPFSSISPDEAVSHIQDGDFVVLSHAAAVPQACVAALARNYERFHDVRIFHMVTLGESPYTAPEMEGHFRLVTNFVGANTRPAVDEQRADFVPSYFYEVPSMMQPGGVFHPDVAIVQLSYPNEEGYCSFGTSCDYSKPAAEQARVVIGELNKQMPFVGGDNLIHISQLTHIVEADYPLYALDLPRIGEVEEAIGRNCAELIQDGDTLQLGIGAIPDAVLLFLKDKKDLGIHSEMVSDGVVKLIKAHTITGRCKTLLPNKVVATFLMGTQELYDFAHNNPAIEMRPVNYVNDPRIIAQNDHLVSINSCIEVDLMGQVVSEAMGLRQFSGPGGQVDFVRGAAWSKGGRSIIAIPSTARRGTASRIVPLLTEGAAITTSRNDVDYIVTEYGVAHLKGKSLRERALALTAIAHPNFRPELEAECKRRFKL

SEQ ID NO: 268 - protein, 434 aa, Fusobacterium nucleatum
MKNWKESYKTKICTPDEAIQKIKDAKRISFGHICSESTVLTEALIRNKKLFKKLEIAHLLSVGKSEYAKEENSEYFRHNALFIGPNTRETANSSYGDYTPTFFFETAKLFGKDGELALDAMLLQVSTPDEHGYCSYGLSCDYTKSATENAKIVIAQINKFVPRTLGNCFVHIDDIDYIIEEDTPIPEVQPPVVGETERKIGEFCASLVRDGDTLQLGIGAIPVAVLNFLKDKKDLGIHSEMISDGIVDLINLGVITNKKKNLNPNKAIATFLMGSKKLYDYADNNPAIELHPVDYVNNPIIIAQNDNMVSINSAIQIDLMGQVNAEYVDSKQFSGPGGQVDFVRGATMSKGGKSIIALPSTTAKGTISRIVFTFDEGVPVTTSRNDVDYVITEYGIAHLRGKTLRERAKLLIEIAHPNFREELRKKALEKFGEL

SEQ ID NO: 269 - protein, 445 aa, Geobacter bemidjiensis
MAATIWKEIKNWREIYNSRVTTADEALKSVNSGDRLYLTGNASVPLQLLDALVRRAPELRDVEVSQVLTICPQDYVSPEMQGHVRVNTMFISPNVRQAVNEGRADFTPVLLSEFPLLFKNGHLPLDVALLNVSLPDENGFCSLGGEAGLTMTAAESARIVIAQVNKKMPRTLGDTFFHVSRMTHIVEVDAPLTERHMAEEGDPEIVEQIAGHISSLIPDGATMQLGIGAIPDAVLKHLFSKKDLGIHSELISDGVIDLVEAGVVNCSRKTLHPGKIVCGFLLGTKRLYDWAHNNPMIELRRTEYVNDPFVVAQNDRMVSVNAAIEVDFTGQICADSIGPRMYSAVGGQMDFVYGASRSKGGVAVITMPSTSTLRDGSVVSKITPLLKHGAGVVTSRNHVRYLVTEYGAADLYAKSVRQRTQALIGVAHPMFREELTEKAKELRYI

SEQ ID NO: 270 - protein, 434 aa, Fusobacterium sp.
MKNWKESYKTKICTPDEAIQKIKDAKRISFGHICSESTVLTEALIRNKKLFKKLEIAHLLSVGKSEYAKEENSEYFRHNALFIGPNTREAANSSYGDYTPTFFFETAKLFGKDGELALDAMLLQVSTPDEHGYCSYGLSCDYTKSATENAKIVIAQINKFVPRTLGNCFVHIDDIDYIIEEDTPIPEVQPPVVGETERKIGEFCASLVRDGDTLQLGIGAIPVAVLNFLKDKKDLGIHSEMISDGIVDLINLGVITNKKKNLNPNKAIATFLMGSKKLYDYADNNPAIELHPVDYVNNPIIIAQNDNMVSINSAIQIDLMGQVNAEYVDSKQFSGPGGQVDFVRGATMSKGGKSIIALPSTTAKGTISRIVFTFDEGVPVTTSRNDVDYVITEYGIAHLRGKTLRERAKLLIEIAHPNFREELRKKALEKFGEL

SEQ ID NO: 271 - protein, 434 aa, Fusobacterium sp.
MKNWKESYKTKICTPDEAIQKIKDAKRISFGHICSESTVLTEALIRNKKLFKKLEIAHLLSVGKSEYAKEENSEYFRHNALFIGPNTREAANSSYGDYTPTFFFETAKLFGKDGELALDAMLLQVSTPDEHGYCSYGLSCDYTKSATENAKIVIAQINKFVPRTLGNCFVHIDDIDYIIEEDTPIPEVQPPVVGETERKIGEFCASLVRDGDTLQLGIGAIPVAVLNFLKDKKDLGIHSEMISDGIVDLINLGVITNKKKNLNPNKAIATFLMGSKKLYDYADNNPAIELHPVDYVNNPIIIAQNDNMISINSAIQIDLMGQVNAEYVDSKQFSGPGGQVDFVRGATMSKGGKSIIALPSTTAKGTISRIVFTFDEGVPVTTSRNDVDYVITEYGIAHLRGKTLRERAKLLIEIAHPNFREELRKKALEKFGEL

SEQ ID NO: 272 - protein, 435 aa, Eubacterium yurii
MDVKKVYEEKKMTAQEAVKKIKSGDRVIVGHACGEPACLLNAMVDNKDDYENVEIVHMVAMGKARYCEEGMEKHFRHNALFVGGTTKKAVNEARADFTPTFFFEVPKLFREKLPVDVVLAQVSPMDDDGYMSLGISVDYTKPACEIAKTVILQVNKNMPRTMGDSLIHISNATCIVECDETIYTLPRPAIGELEKKIGENCAKLIRDGDTLQLGIGAIPDAVLSFLTDKKDLGIHSEMFTDGVVELVKKGVVTNKMKKTHTGKMIATFLMGTRELYDFVDNNHDIAMFPVDYVNHPAIIMKEDNMVSINSCVQVDLMGQVVSESIGKTQISGVGGQVDFVRGASMAKNGRSIIAMPSTVKDKSSKIVVKIDDYAAVTTSRNDVDYVVTEYGIAELKGKTLKQRSENLIKIAHPKFREELIKYHNEKFNDNLKLEN

SEQ ID NO: 273 - protein, 431 aa, Roseiflexus castenholzii; identical to SEQ ID NO: 189

SEQ ID NO: 274 - protein, 434 aa, Fusobacterium nucleatum
MKNWKESYKSKICTPDEAIQKIKGAKRISFGHICSESTVLTEALVRNKKLFKKLEIAHLLSVGKSEYAKEENSEYFRHNALFIGPKTREAANSSYGDYTPTFFFETAKLFQKDEELALDAMLLQVSTPDEHGYCSYGLSCDYTKSATENAKIVIAQINKFVPRTLGNCFVHIDNIDYIIEEDTPIPEVQPPVVGEIERKIGGFCASLVRDGDTLQLGIGAIPVAVLNFLKDKKDLGIHSEMISDGIVDLINLGVITNKKKNLNPNKAIATFLMGSKKLYDYANDNPAIELHPVDYVNNPIIIAQNDNMVSINSAIQVDLMGQVNAEYIDSKQFSGPGGQVDFVRGATMSSGGKSIIALPSTTGKGTISRIVFTFDEGVPVTTSRNDVDYVITEYGIAHLRGKTLRERAKLLIEIAHPDFREELRKKALEKFGEL

SEQ ID NO: 275 - protein, 428 aa, Porphyromonas asaccharolytica
MSIPFSSISPDEAVSHIQDGDFVVLSHAAAVPQACVAALARNYERFHDVRIFHMVTLGESPYTAPEMEGHFRLVTNFVGANTRPAVDEQRADFVPSYFYEVPSMMQPGGVFHPDVAIVQLSYPNEEGYCSFGTSCDYSKPAAEQARVVIGELNKQMPFVGGDNLIHISQLTHIVEADYPLYALDLPRIGEVEEAIGRNCAELIQDGDTLQLGIGAIPDAVLLFLKDKKDLGIHSEMVSDGVVKLIKAHTITGQCKTLLPNKVVATFLMGTQELYDFAHNNPAIEMRPVNYVNDPRIIAQNDHLVSINSCIEVDLMGQVVSEAMGLRQFSGPGGQVDFVRGAAWSKGGRSIIAIPSTARRGTASRIVPLLTEGAAITTSRNDVDYIVTEYGVAHLKGKSLRERALALTAIAHPNFRPELEAECKRRFKL

SEQ ID NO: 276 - protein, 425 aa, Clostridium beijerinckii
MHQFVSADQAVKAIKSGDRIYVQAAAAAPSVLIEALTKRASELQNVEICHLHTEGPAPYADPSLRDSFHVNSFFIGKNVRHTLKAGNGSYTPVFLSELPNVFRRGIVTLDVAFVHVSPPDSHGYCSLGVSVEASIAAIESAKIVIAQVNNQMPRTFGDGIIHVSEINYLVEANVPIFEHGMAEATEVELQIGRNIATLIEDGATLQMGIGSIPNAALLQLTNHKNLGLHTEMFSDGVIELIEKNVINCNYKVVNRGRALATFLLGSKRLYDFVNDNPFIEMRESSYVNDTAVIRQNPKTIAINSAIEVDLTGQVCADSIGSTMYSGVGGQMDFIRGASLSPGGKAIIALPSVTRRGESRIVSYLKTGAGVVTTRSHVQYIITEYGIAELYGKSLQQRAKALINIAHPAHREQLDKDFFSVQRSYV

SEQ ID NO: 277 - protein, 434 aa, Fusobacterium nucleatum
MKNWKESYKSKICTPDEAIQKIKDAKRISFGHICSESTVLTEALVRNKKLFKKLEIAHLLSVGKSEYAKEENSEYFRHNALFIGPKTREAANSSYGDYTPTFFFETAKLFGKDGELALDAMLLQVSTPDEHGYCSYGLSCDYTKSATENAKIVIAQINKFVPRTLGNCFVHIDDIDYIIEEDTPIPEVQPPVVGEIERKIGEFCASLVRDGDTLQLGIGAIPVAVLNFLKDKKDLGIHSEMISDGIVDLINLGVITNKKKNLNPNKAIATFLMGSKKLYDYANDNPAIELHPVDYVNNPIIIAQNDNMVSINSAIQVDLMGQVNAEYIDSKQFSGPGGQVDFVRGATMSKGGKSIIALPSTTGKGTISRIVFTFDEGTPVTTSRNDVDYVITEYGIAHLRGKTLRERAKLLIEIAHPDFREELRKKALEKFGEL

SEQ ID NO: 278 - protein, 435 aa, Eubacterium sp.
MDVKKLYEEKKMTAQEAVKKIKSGDRVIVGHACGEPACLLNAMVDNKDDYENVEIVHMVAMGKARYCEEGMEKHFRHNALFVGGTTKKAVNEARADFTPTFFFEVPKLFREKLPVDVVLAQVSPMDDDGYMSLGISVDYTKPACEIAKTVILQVNKNMPRTMGDSLIHISNATCIVECDETIYTLPRPEIGELEKKIGENCAKLISDGDTLQLGIGAIPDAVLSFLTDKKDLGIHSEMFTDGVVELVKKGVVTNKMKKTHTGKMIATFLMGTRELYDFVDNNHDIAMFPVDYVNHPAIIMKEDNMVSINSCVQVDLMGQVVSESIGKTQISGVGGQVDFVRGASMAKNGRSIIAMPSTVKDKSSKIVVKIDDYAAVTTSRNDVDYVVTEYGIAELKGKTLKQRSENLIKIAHPKFREELIKYHNEKFNDNLKLEN

SEQ ID NO: 279 - protein, 434 aa, Desulfosporosinus acidiphilus
MDWRETYKSRLTTADEAVKVVKSGDFVIPGHAASESELLVNALVKRYLDLENVTIIQGVALGSSPYCKPEMEGHFILKSMFVGANTRKSVWENRGTFFPLMFHEFPRAFREGYLGSDVFITMVSPPDEHGYCSLGMSVDHSKELVRCAKTVIAEVNPNVPRTYGDTFVHVSDLDYIVENDGPLLELNRFAAMDEVTQAIGRNVAGLINDGDTLQMGAGTIPDAILHYLKDKRDLGIHTEMFSDGLIDLIEAGIVNCSKKTINPGKIVVTFAEGTKKVYEYIDRNPMFQFHPVDYVNNPCIIAQHDNMVAINSALEIDLSGQVCAEALGAKQYSGIGGQLDFIRGAAAAKNGRPVIVLQSTAKSATISRISCQLKPGTPVTTTRNDVHWVATEYGAVDLFCKSEKERAAALISIAHPNFRDELKRQYREIYGRVL

SEQ ID NO: 280 - protein, 431 aa, Flavonifractor plautii
MNWKEYYKQHLMSAEDAVNHIQSGNRVVVAHACGEPSYLLDVMVANAKAYRDVEIVHMVAMGKAEYCKPEYAGNFRHNAFFVGGTSRDAIAEGRGDFTPSFFFEVPRQFSSTMPVDVAMVMVTPPDENGMCSLGVSVDYTLEAVKQAKLVIAQVNPQMPWTGPYSLVSVKDLDCIVEHEAPIIELKPPKIGDIEKAIGEHCASLVPDGATLQLGIGAIPDAVLLFLKHKKDLGIHSEMFSDGVVELAEAGVITNAKKTLHPGKFEVAFLMGTRRLYDFVDHNPDVELRPVDYVNNPFVIAQNENMVSINSCVQVDLMGQVASESIGPKQISGVGGQVDFVRGASASKGGVSIMAMPSTVKGKISKIVPLLDEGAAVTTSRNDVDYIVTEYGVAALKGQTLRQRARNLIEIAHPDFRDELKAEYEKRFHCKY

SEQ ID NO: 281 - protein, 430 aa, Clostridium arbusti
MNWKDIYKDRLVSAKEAVSNIKSGSRVVIGHAVGEPLALVDALVENKKYYDNLEIVHMVAMGKSEYVKPGMEKYFRHNALFVGNSTREAVNSGRADYTPGFFYEVPRLFKEGYLPVDVALIQVSVPDKHGYCSFGVSNDYTKPAADNAKLVIAEVNENMPRTLGDSFIHVSDIDYIVEASHPLIELQPSKIGDVEKAIGKYCASLIEDGSTLQLGIGAIPDAVLLFLKDKKDLGIHSEMISDGVVELVNDGVITNKKKAFHNGKIVVTFLMGTKKLYDFVNDNPMVESYPVSFVNDPETIMKNNDMISINSCIQVDLMGQICSESIGIKQISGVGGQVDFIRGTSMAKNGKAIIAMPSTAAKGKVSRIVPILDEGAAVTTSRNDVDYIITEYGIAHLKGKTLRDRAKDLIDIAHPDFREYLSEELKKRFR

SEQ ID NO: 282 - protein, 433 aa, Clostridium carboxidivorans; identical to SEQ ID NO: 198

SEQ ID NO: 283 - protein, 430 aa, Tannerella forsythia
MEWKTTYRNKIVSADEAVRQIRNNELVSLGHAAGVPQMCVDALVRNSTYFKNVGIYHMLVLGEAKYTEPGMEEAFRHVASFIGANTRSAIKEKRVDFLPAFLSEVPRMIRERLIPVDVAIVQLSPPDEDGNCSFGISSDYSKPSTEHARLVIGEINEQMPYIGGDNLIHLSKLDCIVEADYPLYTIAPPRITEVEEAIGRNCATLIEDGATLQLGIGAIPDAVLLFLKDKKNLGIHSEMFSDGVLELVKNGVINGKAKTLHPNKMVATFLMGTQALYDFVNHNPDVEMYPSDYVNDPRVIAQNDRMISINSCLSVDLAGQVSSESIGLQQISGTGGQVDYVRGSMWSRGGKSIMAMPSTAKNGTTSRIVATLTEGTTVTTSRNDVDYVVTEYGVARLRGKSLRERAKALIKIAHPDFREELEAFYQQRFH

SEQ ID NO: 284 - protein, 442 aa, Anaerostipes sp.
MRDKKDKPWKAEYREKLVSADEAVSHIRSGQRIVFSHAAGESLVLSDALVRNRALFENVEIVHMVAMGEAKYCEPGMEKHFRHNSFFLGASTRKAAKEGRADVTPVYFSEVPELFRTTLPVDAVFLNLSPPDEHGYCSFGISVDYSKPAAMEAELVIAQINPSMPRTLGDSFIHISDIDYIVEADTPLIELPPAGISEVERAIGKNCASLIEDGDTLQLGIGAIPDAVLGFLKEKKDLGIHSEMISDGIVELYEGGVITNRRKSLHAGKSIVTFLMGTRRLYDFADNNPAVELHPVDYVNDPYVIAQNERLVSVNSCVQVDLMGQVFSASVGRRQISGVGGQVDFVRGANMSRGGKSIMAMPSTAAEGRISKIVPVIGEGAAVTTSRYDADYIVTEYGAARLKGESLRNRAKKLIRIAHPDFRRMLAEEYEKRFREAWSDDE

SEQ ID NO: 285 - protein, 435 aa, Fusobacterium sp.
MENWKEIYKEKIVTADEAVKYIQSGNRVIFAHACGEAQIITEAMLRNKEEYEKVEIVHLVPMGTGEYAQEENQKYFYHNSFFGGGSTRKALNGTYGDFTPSFFFEIPKLFKKDGKLPVDVAIIQVSSPDEHGYCSYGISCDYTKGAAENAKIVIAQVNKYMPRTLGDSFIHLSEMDYIVAYDEPILQLVPPKIGEIEQKIGEYCASLIQDGDTLQLGIGAIPDAVLTFLKDKRHLGIHSEMISDGVVDLILAGVIDNSKKTIHKNKCVVSFLMGSPKLYDFVNNNPAVELYPVDYVNHPMIIAKNDNMISINSALQVDLMGQVNAESIGAKQFSGTGGQVDFVRGASMSKGGKSIIAMPSTAAKGRISKIVMNLDLGSTVTTSRNDVDYVVTEYGIASLKGKTLRERAKALIAIAHPDFRESLMKQALEKFQILN

SEQ ID NO: 286 - protein, 445 aa, Geobacter sp.
MAATIWKEIKNWREIYNSRVTTADEALKSVNSGDRLYLTGNASVPMQLLEALVKRAPELRDVEVSQVLTICPQDYVSPEMEGHVRVNTMFISPNVRQAVNEGRADFTPVLLSEFPLLFKHGHLPLDVALLNVSLPDENGFCSLGGEAGLTMTAAESARIVIAQVNKKMPRTLGDTFFHVSRMTHIVEVDAPLTERHMAEEGDPQIVEQIAGHISSLIPDGATMQLGIGAIPDAVLKHLFSKKDLGIHSELISDGVIDLVEAGVVNCSRKTLHPGKIVCGFLLGTKRLYDWAHNNPMIELRRTEYVNDPFVVAQNDRMVSVNAAIEVDFTGQICADSIGPRMYSAVGGQMDFVYGASRSKGGVAVITMPSTSTMRDGSVVSKITPLLKHGAGVVTSRNHVRYLVTEYGAADLYAKSVRQRTQALIGVAHPMFRDELTEKAKELRYI

SEQ ID NO: 287 - protein, 432 aa, Desulfosporosinus acidiphilus
MKWQTVYDGRIKEAAEAVRIIKSGQRVVIGHACGEPQALVEAMAERKSELQSVEIVHMVAMGKASYCLPEMEGHFRHNSLFVGGTSRKAVNEGRADFTPVFFSEIPSLFRDGYLPPDVALVQMSPPDQSGFCSLGVSVDYTLQALRSAHTVIAQVNHFMPRTLGDSFVHVSELDIIVPFDEPLIELPKPQISQVEENIGRFVAGLVPDGATLQLGIGAIPDAVLRFLTNKNDLGIHSEMFSDGVVNLYEMGVINNKAKTLHPGKMLATFLMGTRVLYDFVNDNPVVEMHSVDYTNNPYIIGRNERLTSINSALQVDLCGQVCADSIGYRQYSAVGGQVDFVRGARLSCGGKSIIALPSTAANGKISRIAIHLDEGAAVTTSRNDVDYIVTEYGIASLRGKTIRERANALIEIAHPQFHEELKVQAKKNGIPL

SEQ ID NO: 288 - protein, 429 aa, Clostridium kluyveri
MEWEEIYKEKLVTAEKAVSKIENHSRVVFAHAVGEPVDLVNALVKNKDNYIGLEIVHMVAMGKGEYTKEGMQRHFRHNALFVGGCTRDAVNSGRADYTPCFFYEVPSLFKEKRLPVDVALIQVSEPDKYGYCSFGVSNDYTKPAAESAKLVIAEVNKNMPRTLGDSFIHVSDIDYIVEASHPLLELQPPKLGDVEKAIGENCASLIEDGATLQLGIGAIPDAVLLFLKNKKNLGIHSEMISDGVMELVKAGVINNKKKTLHPGKIVVTFLMGTKKLYDFVNNNPMVETYSVDYVNNPLVIMKNDNMVSINSCVQVDLMGQVCSESIGLKQISGVGGQVDFIRGANLSKGGKAIIAIPSTAGKGKVSRITPLLDTGAAVTTSRNEVDYVVTEYGVAHLKGKTLRNRARALINIAHPKFRESLMNEFKKRF

SEQ ID NO: 289 - protein, 432 aa, Thermoanaerobacter siderophilus
MNWHDEYIRKVVSPDEAVKHIKSGDRVVIQHACGEPQLLVDAMVKNADMYENVEIVHMVAMGKAEYTKPGMEKHFWHNSLFLGATTREAINEGRGDYTPCFFFEIPNLFRNNILPVDVALIQVSKPDRHGYCSLGVSCDYTKAAVECAKIVIAEVNEKMPRTGGDCFVHVSDIDYIVEASEPIIELKRAKIGDVEKKIGENCAKLIEDGSTLQLGIGGIPDAVLSFLKDKNDLGIHSEMFSDGVVELVENGNITNKAKTLHKGKFIATFLMGSQKLYDFVNDNPAVGLYPVDYVNNPYIIAQNHRMVSINSAIQIDLMGQVAAESIGIKQFSGTGGQVDFVRGAALAKDGKAIIAMPSTAAGGKVSRIVPYLDEGAAVTTSRNDVQYVVTEYGIADLRGKTLRERARELINIAHPKFRDFLLEKAKERFKNF

SEQ ID NO: 290 - protein, 434 aa, Fusobacterium gonidiaformans
MTHWKGLYQERLCSAEQAVKSIPNNCRVVPSHAAGEPKHLVEAMMANREQYHNVDIFSMVNLGHAAYGKEEEKEHFHVNAAYASASTREVVNAEHGDFTPCFFYQVPELLKKDGPMPADVALIQVSLPDEHGYCSLGVSSDYTKEAAENAKIVIAQVNKYMPRTLGNNFVHVSKMTHIVEYDEPIHILNPPFVGETERKIGEYCASLIQDGDTLQLGIGAIPDAVLSFLTDKKHLGIHSEMISDGVVDLIEAGVIDNSRKNFNPGKSIVSFLMGTEKLYNYVHNNPALEMHPVDYVNHPIIAAQNDNLVSINSALQVDLMGQANSETLGHKQFTGIGGQVDFVRAASMSKGGRTIIAMPSTAAKGKISKIVFLLDEGAAVTTSRTDIDYVITEYGIAKLRGKSLRARAKALIEIAHPDFREGLREQALQKFGRL

SEQ ID NO: 291 - protein, 433 aa, Fusobacterium varium
MDWKEEYKKRVYSPEEAVKKIKSNNRVVICHAAGEPQTLVDAMVKNYKEYENVEIVHMVPLGKCEYMKPEMAGHFRHNGLFLGGGTREAVSAGAGEYTPSFFFEIPKLFRKGGNLPVDVALIQVSLPDEHGYCSLGITCDYTKAAAENAKCVIAQVNKFMPRVLGDNFIHISEIDAIVEKDEPIIVLNPPKIGDVERKIGEYCASLINDGDTLQLGIGGIPDAVLTFLKDKKDLGIHSEMISDGVVDLVKAGVINNSKKTLHRGKSIVSFLMGTRKLYDYVDDNPSVEIYPVDYVNDPRVISKNDNIVSINSAIQVDLMGQVNAETIGTKQFSGTGGQVDFVRGTAFALNGKSIIAIPSTASKGKVSRITAYLDEGAAVTTSRNDVQYIITEYGIADLRGKTLRERAKELIKIAHPDFREELTQKALEKYKTL

SEQ ID NO: 292 - protein, 436 aa, Fusobacterium necrophorum; identical to SEQ ID NO: 191

SEQ ID NO: 293 - protein, 443 aa, Anaerostipes caccae
MMRDKKDKPWKAEYREKLVSADEAVSHIRSGQRIVFSHAAGESLVLSDALVRNRALFENVEIVHMVAMGAAKYCEPGMEKHFRHNSFFLGASTRKAAKEGRADVTPVYFSEVPELFRTTLPVDAVFLNLSPPDEHGYCSFGISVDYSKPAAMEAELVIAQINPSMPRTLGDSFIHISDIDYIVEADTPLIELPPAGISEVERAIGKNCASLIEDGDTLQLGIGAIPDAVLGFLKEKKDLGIHSEMISDGIVELYEAGVITNRRKSLHAGKSIVTFLMGTRKLYDFADNNPAVELHPVDYVNDPYVIAQNERLVSVNSCVQVDLMGQVVSASVGRRQISGVGGQVDFVRGANMSRGGKSIMAMPSTAAEGRISKIVPVIGEGAAVTTSRYDADYIVTEYGTARLKGETLRNRARKLIRIAHPDFRRMLAEEYEKRFREAWSDDE

SEQ ID NO: 294 - protein, 432 aa, Clostridium sp.
MDWKDLYAQRKMTAEEAVARVRSGDRVVTPNAAGAPVHLINALVARKDDLKNVEIAHMVPLAKAPYAAPEMEGHFIHNSIFVGGSTRATIEAGIGDYTPCFFHKVPELFDTTLPVDVALIHVSAPDEHGYCSYGVTVDYEKHAAEVAKLVIAQVNENMPRTLGDCFIHVSEIDCIVECNDPILELGAPKITEIEKKIGGHCAELIKDGDTLQLGIGAIPDAVLLFLKEKKDLGIHTEMFSDGVVELVEAGVINNSKKTLHKGKSVATFLMGTRRLYDYVHNNPAVEMYPVNYVNDPVVIAKNDNLVSINSCVQIDLLGQVVSESIGPVQISGVGGQIDFIRGASMSRGGRSILAMNSTAAGGKVSKIVPFIDPWSAVTTSRNDVQYVVTEYGIAELRGHTMRDRARSLIEIAHPDFRDPLKEEFEKRFHTKY

SEQ ID NO: 295 - protein, 432 aa, Anaerofustis stercorihominis
MDLKQIYNSKVKNADDAVKAIKSGNRVVIAHAVGEPVYVVDKMVEHHKDYKDVEIVQLVPMGHSLYAKEEMKEHFRLNAFFLGGNTRDAVFEGRGDFTPCFFYQIPQLFRSCLPIDVALITVSPPDENGFCSFGTSCDYTKPAAEAAKIVIAQVNKKMPRTLGDGFIHINDIDIIVEHDEQVPELPNPKITDVEMKIGENCAKLINDGDTLQLGIGAIPDAVLKFLTHKKDLGIHSEMISDGVMDLYEKGVITCKKKNLHKGKMVVAFLMGTQKLYEFVHDNPAISMMDIEYVNNPIVIGQNDNMVSVNSALQVDLMGQVCAEAMGLKQFSGIGGQVDFIRGAAFSKGGKSILAFPSTAKKGTISKIVPFLTEGSAVTTSRTDVDYIVTEHGIAHLKGQTLKERARRLINIADPNFRDGLIAEFENRFKENF

SEQ ID NO: 296 - protein, 442 aa, Anaerostipes caccae
MRDKKDKPWKAEYREKLVSADEAVSHIRSGQRIVFSHAAGESLVLSDALVRNRALFENVEIVHMVAMGAAKYCEPGMEKHFRHNSFFLGASTRKAAKEGRADVTPVYFSEVPELFRTTLPVDAVFLNLSPPDEHGYCSFGISVDYSKPAAMEAELVIAQINPSMPRTLGDSFIHISDIDYIVEADTPLIELPPAGISEVERAIGKNCASLIEDGDTLQLGIGAIPDAVLGFLKEKKDLGIHSEMISDGIVELYEAGVITNRRKSLHAGKSIVTFLMGTRKLYDFADNNPAVELHPVDYVNDPYVIAQNERLVSVNSCVQVDLMGQVVSASVGRRQISGVGGQVDFVRGANMSRGGKSIMAMPSTAAEGRISKIVPVIGEGAAVTTSRYDADYIVTEYGTARLKGETLRNRARKLIRIAHPDFRRMLAEEYEKRFREAWSDDE

SEQ ID NO: 297 - protein, 438 aa, Clostridium beijerinckii; identical to SEQ ID NO: 197

SEQ ID NO: 298 - protein, 438 aa, Clostridium beijerinckii; identical to SEQ ID NO: 197

SEQ ID NO: 299 - protein, 448 aa, Clostridium beijerinckii
MDWKKIYEDRTCTADEAVKSIKSGDRVLFAHCVAEPPVLVEAMVANAAAYKNVTVSHMVTLGKGEYSKPEYKENFTFEGWFTSPSTRGSIAEGHGQFVPVFFHEVPSLIRKDIFHVDVFMVMVSPPDHNGFCCVGVSSDYTMQAIKSAKIVLAEVNDQVPVVYGDTFVHVSEIDKFVETSHPLPEIGLPKIGEVEAAIGKHCASLIEDGSTLQLGIGAIPDAVLSQLKDKKHLGIHSEMISDGVVDLYEAGVIDCSQKSIDKGKMAITFLMGTKRLYDFAANNPKVELKPVDYINHPSVVAQCSKMVCINACLQVDFMGQIVSDSIGTKQFSGVGGQVDFVRGASMSIDGKGKAIIAMPSVAKKKDGSMISKIVPFIDHGAAVTTSRNDADYVVTEYGIAEMKGKSLQDRARALINIAHPDFKDELKAEFEKRFNAAFSAWSHPQFEK

SEQ ID NO: 300 - protein, 438 aa, Eubacterium saphenum; identical to SEQ ID NO: 195

SEQ ID NO: 301 - protein, 435 aa, Clostridium difficile
MSWRELYQSKLCSATEAVKQIKNGDTVVFAHCVGEPPALVEAMIENAEQYKDVEIKHMVSLGSGGYTAKGMEAHFRVNPMFVSGNVRKAIENGDGDFTPTFFHEVPKLLREKRLKCDVVLAQVTPPDEHGYCSLGTSVDYTYEAIKTARTVIVQVNDQFPRTYGEVVHVSEFDYIVEKSQPLFELQPAKIGEVEEAIGKNCASLIEDGSTLQLGIGGIPDAVMLFLTDKKDLGIHSEMISDGTLALYEKGVINGKYKNFDKEKMTVTFLMGTKKLYDFANNNPAVEVKPVDYVNHPAIIMKQHKMVSINSAIQVDLMGQVVAEAMGLRQFSGVGGQVDFIRGVSMGEDGKAIIAMPSITTKKDGTVISKIVSIVDEGAPITTSRNDVDYIVTEYGIAELKGKSLRERARNLINIAHPSVRESLAVEFEKRFKEKY

SEQ ID NO: 302 - protein, 435 aa, Clostridium difficile
MSWQELYQSKLCSATEAVKQIKNGDTVVFAHCVGEPPALVEAMIENAEQYKDVEIKHMVSLGSGGYTAKGMEAHFRVNPMFVSGNVRKAIENGDGDFTPAFFHEVPKLLREKRLKCDVVLAQVTPPDEHGYCSLGTSVDYTYEAIKTARTVIVQVNDQFPRTYGEVVHVSEFDYIVEKSQPLFELQPAKIGEVEEAIGKNCASLIEDGSTLQLGIGGIPDAVMLFLTDKKDLGIHSEMISDGTLALYEKGVINGKYKNFDKEKMTVTFLMGTKKLYDFANNNPAVEVKPVDYVNHPAIIMKQHKMVSINSAIQVDLMGQVVAEAMGLRQFSGVGGQVDFIRGVSMGEDGKAIIAMPSITTKKDGTVISKIVSIVDEGAPITTSRNDVDYIVTEYGIAELKGKSLRERARNLINIAHPSVRESLAVEFEKRFKEKY

SEQ ID NO: 303 - protein, 435 aa, Clostridium difficile
MSWQELYQSKLCSATEAVKQIKNGDTVVFAHCVGEPPALVEAMIENAEQYKDVEIKHMVSLGSGGYTAKGMEAHFRVNPMFVSGNVRKAIENGDGDFTPAFFHEVPKLLREKRLKCDVVLAQVTPPDEHGYCSLGTSVDYTYEAIKTARTVIVQVNDQFPRTYGEVVHVSEFDYIVEKSQPLFELQPAKIGEVEEAIGKNCASLIEDGSTLQLGIGGIPDAVMLFLTDKKDLGIHSEMISDGTLALYEKGVINGKYKNFDKEKMTVTFLMGTKKLYDFANNNPAVEVKPVDYVNHPAIIMKQHKMVSINSAIQVDLMGQVVAEAMGLRQFSGVGGQVDFIRGVSMGEDGKAIIAMPSITTKKDGTVISKIVSIVDEGAPITTSRNDVDYIVTEYGIAELKGKSLRERARNLIDIAHPSVRESLAVEFEKRFKEKY

SEQ ID NO: 304 - protein, 435 aa, Clostridium difficile
MSWRELYQSKLCSATEAVKQIKNGDTVVFAHCVGEPPALVEAMIENAEQYKDVEIKHMVSLGSGGYTAKGMEAHFRVNPMFVSGNVRKAIENGDGDFTPTFFHEVPKLLREKRLKCDVVLAQVTPPDEHGYCSLGTSVDYTYEAIKTARTVIVQVNDQFPRTYGEVVHVSDFDYIVEKSQPLYELEPAKIGEVEEAIGKNCASLIEDGSTLQLGIGGIPDAVMLFLTDKKDLGIHSEMISDGTLALYEKGVINGKYKNFDKEKMTVTFLMGTKKLYDFVNNNPAVEVKPVDYVNHPAIIMKQHKMVSINSAIQVDLMGQVVAEAMGLRQFSGVGGQVDFIRGVSMGEDGKAIIAMPSITTKKDGTVISKIVSIVDEGAPITTSRNDVDYIVTEYGIAELKGKSLRERARNLINIAHPSVRESLAIEFEKRFKEKY

SEQ ID NO: 305 - protein, 433 aa, Clostridium sp.; identical to SEQ ID NO: 196

SEQ ID NO: 306 - protein, 433 aa, Clostridium arbusti
MNWKDVYKNRLASAKKAVSKIESNSRIVVSHAVGEPLDLMNALVENRNEYKNLEIVHMLAMGKGEYVKPEMKKFFHHNALFVGTSTMEAVKSGRAEYTPCFFYEVPRLFKEGYLSVDVALIQVSEPDQYGYCSFGVSNDYSKPAAESAKLVIAEVNKNMPRILGDSFIHVSDIDYIVESSHPLIELKPPKIGDVEKAIGKYCASLIEDGSTLQLGIGAIPDAVLLFLKDKKDLGIHSEMISDGVVELVEAGVINNKKKTLHKGKIVVTFLMGTKKLYDFVDNNPMVESYSVDYVNNPMVIMKNYKMVSINSCIQVDLMGQVCSESIGLNQISGVGGQVDFIRGASMAEYGKAIIAIPSTAAHGKVSRIVPFLNEGAAITTSRNEVDYIITEYGIAHLKGKTLSDRAQNLINIAHPDFRESLITEFEKRFNCKF

SEQ ID NO: 307 - protein, 436 aa, Clostridium beijerinckii; identical to SEQ ID NO: 190

SEQ ID NO: 308 - protein, 431 aa, Clostridium beijerinckii
MNWKEYYKQHLMSAEDAVNHIQSGNRVVVAHACGEPSYLLDVMVANAKAYKDVEIVHMVAMGKAEYCKPEYAENFRHNAFFVGGTSRDAIAEGRGDFTPSFFFEVPRQFSSTMPVDVAMVMVTPPDENGMCSLGVSVDYTLEAVKQAKLVIAQVNPQMPWTGPYSLVSVKDLDCIVEHEAPIIELKPPKIGDIEKAIGEHCASLVPDGATLQLGIGAIPDAVLLFLKHKKDLGIHSEMFSDGVVELAEAGVITNAKKTLHPGKFEVAFLMGTRRLYDFVDHNPDVELRPVDYVNNPFVIAQNENMVSINSCVQVDLMGQVASESIGPKQISGVGGQVDFVRGASASKGGVSIMAMPSTVKGKISKIVPLLDEGAAVTTSRNDVDYIVTEYGVAALKGQTLRQRARNLIEIAHPDFRDELKAEYEKRFHCKY

SEQ ID NO: 309 - protein, 436 aa, Clostridium beijerinckii; identical to SEQ ID NO: 194

SEQ ID NO: 310 - protein, 429 aa, Clostridium kluyveri; identical to SEQ ID NO: 192

SEQ ID NO: 311 - protein, 430 aa, Odoribacter splanchnicus
MEWKEIYKNRFATVEEAVRAIEDGDRLVFGHAAGCPQVVPAEMIAQKERLHDVGIYHMLTLNDGSYLTPGTEGHFRHITNFVGGNSRQAVADDRADFLPVFFYEIPQLFDEVYPVDVAVIQVSYPDSEGNCSFGLSCDYTKAAAEKARLVIAEMNENMPYVEGDNKIHISRLDYIIPTNLALPEIPLPKITDVEKAIGRNCASLIEDGSTLQLGIGAIPDAVLLFMGDKKNLGIHTEMFSDGVIDLVESGVVNGSKKTLHPGKLVATFLMGTRRLYDFVDKNACVEMRPVDYVNDPRVIAQNEKMVSINSCIEVDLMGQVASETIGLKQFSGTGGQVDYVRGAAWSAGGKSIMAMPSTAAKGKASRIVPFLAQGAAVTTSRNDVDYVVTEYGIARLKGKTLKQRARALIAVAHPDFRPMLEEEYQKRFKD

SEQ ID NO: 312 - protein, 431 aa, Porphyromonas gingivalis
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEIDLMGQVVSECIGSKQFSGTGGQVDYVRGASWSKNGKSIMAIPSTAKNGTASRIVPIIAEGAAVTTLRNEVDYVVTEYGIAQLKGKSLRQRAEALIAIAHPDFREELTEHLRKRFG

SEQ ID NO: 313 - protein, 433 aa, Fusobacterium ulcerans
MDWREDYKKKVCSPEEAVKKIKSDSRVVICHAAGEPQTVVDAMVQNYKEYKNVEIVHMVALGKCEYMKPEMAGHFRHNGLFLGGGTRDVVNAGNGDYTTSFFFEIPKLFKKGGSMPVDVALIQVSTPDEHGYCSMGITCDYTKAAAENAKCVIAQVNKFMPRVLGDNFIHISEIDVIVERDEPIIVLNPPRIGDIERKIGEYCASLINDGDTLQLGIGAIPDAVLTFLKDKKDLGIHSEMISDGVVELVKAGVINNSRKTLHKGKSIVSFLMGTRKLYDYVDDNPSVEIYPVDYVNDPRVISKNDNIVSINSAIQVDLMGQVNAETMGTKQFSGTGGQVDFVRGTAFALNGKSVIAIPSTASKGKVSRITAYLDEGAAVTTSRNDVQYIITEYGIADLKGKTLRERAKELIKIAHPDFREELAQKALEKYKTL

SEQ ID NO: 314 - protein, 434 aa, Fusobacterium sp.
MENWKERYESKLCTSDEAIKKIAGVKRIIFEHACGESVLLTEALMKNKELFKKTEIIHLVAMGKGEYAKKENSEYFRHNALFAGGTTREAANSSYGDYTPSFFFEMPELFKKGGVLNPDVTIIQVSYPDEHGYCSYGISCDYTKCAAENSNIVIAQVNKFMPRTLGNCFIHIDNINYIVEQATPIIELKIPVIGEIEKRIGEHCASLIDDGATLQLGIGAIPDAVLSFLRHKKDLGIHSEMISDGVVDLVNLGVITNKRKNINTGKSIVSFLMGTRKLYDYIDNNPEIELHPVDYVNNPFIIAQNDNMISINSAIQVDLMGQVNAESIGSKQFSGTGGQVDFVRGAAMSKGGKSIIALPSTAAKGTISKIVFTLDEGAAVTTSRNDVDYIVTEYGIAHLKGKSLRERAKALIEIAHPDFREELKRKAVGKFGTL

SEQ ID NO: 315 - protein, 434 aa, Fusobacterium nucleatum
MGNWKERYESKLCTSDEAIKKIAGVKRIIFEHACGESALLTEALMKNKELFKKTEIIHLVAMGKGEYAKEENSKYFRHNALFAGGTTREAANSSYGDYTPSFFFEMPELFKKGGVLNPDVTIIQVSYPDEHGYCSYGISCDYTKCAAENSNIVIAQVNKFMPRTLGNCFIHIDNIDYIVEQATPIIELKIPVIGEIEKRIGEHCASLIEDGATLQLGIGAIPDAVLSFLRHKKDLGIHSEMISDGVVDLVNLGVITNKKKNINTGKSIVSFLMGTRKLYDYIDNNPEIELHPVDYVNNPFIIAQNDNMISINSAIQVDLMGQVNAESIGSKQFSGTGGQVDFVRGAAMSKGGKSIIALPSTAAKGTISKIVFTLDEGAAVTTSRNDVDYIVTEYGIAHLKGKSLRERAKALIEIAHPDFREELTKKAVGKFGTL

SEQ ID NO: 316 - protein, 434 aa, Fusobacterium nucleatum
MGNWKERYESKLCTSDEAIKKIAGVKRIIFEHACGESALLTEALMKNKELFKKTEIIHLVAMGKGEYAKEENSEYFRHNALFVGGTTREAANSSYGDYTPSFFFEMPKLFKKGGVLNPDVTIIQVSYPDEHGYCSYGISCDYTKCAAENSNIVIAQVNKFMPRTLGNCFIHIDSIDYIVEQATPIIELKIPVIGEIEKRIGEHCASLIDDGATLQLGIGAIPDAVLSFLRHKKDLGIHSEMISDGVVDLVNLGVITNKRKNINVGKSIVSFLMGTRKLYDYIDNNPEIELHPVDYVNNPFIIAQNDNMISINSAIQVDLMGQVNAESIGSKQFSGTGGQVDFVRGAAMSKGGKSIIALPSTAAKGTISKIVFTLDEGAAVTTSRNDVDYIVTEYGIAHLKGKSLRERAKALIEIAHPDFREELTKKAVGKFGTL

SEQ ID NO: 317 - protein, 436 aa, Fusobacterium sp.
MSNNWREVYKDKIVTADEAVKQIKSGDRLIFSHACGEAQEITDALLRNKESYEKVEIIHLVPMGKGEYAQEENQKYFYHNSFFGGGSTRKAINGAYGDFTPSFFFEIPNLFRKNGKLPLDVAIIQVSAPDEHGYCSYGISCDYTKGAAENAKIVIAQVNKYMPRTLGENFIHLSAIDSIVEYDQPILQLNPPKIGEVEKKIGEYCAGLIQDGDTLQLGIGAIPDAVLTFLKEKKHLGIHSEMISDGVVDLILAGVIDNSQKTIHKNKCIVSFLMGSQKLYDYVHNNPGVELYPVDYVNHPFVIAQNDNMVSINSALQVDLMGQVNAESMGAKQFSGTGGQVDFVRGAAMSKGGRSIIAMPSTAAKGTISKIVMNLDVGATVTTSRNDVDYIITEYGIAELKGKTLRERAKALIAIAHPDFREQLTKQALEKFQRLE

SEQ ID NO: 318 - protein, 431 aa, Porphyromonas gingivalis
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEVDLMGQVVSECIGSKQFSGTGGQVDYVRGAAWSKNGKSIMAIPSTAKNGTTSRIVPIIAEGAAVTTLRNEVDYVVTEYGIAQLKGKSLRQRAEALIAIAHPDFREELTEHLRKRFG

SEQ ID NO: 319 - protein, 431 aa, Porphyromonas gingivalis; identical to SEQ ID NO: 56

SEQ ID NO: 320 - protein, 437 aa, Clostridium kluyveri
MVFKNWQDLYKSKIVSADEAVSKVSCGDSIILGNACGASLTLLDALAANKEKYKSVKIHNLILNYKNDIYTDPESEKYIHGNTFFVSGGTKEAVNCNRTDYTPCFFYEIPKLLKQKYINADVAFIQVSKPDSHGYCSFGVSTDYSQAMVQSAKLIIAEVNDQMPRVLGDNFIHISDMDYIVESSRPILELTPPKIGEVEKTIGKYCASLVEDGSTLQLGIGAIPDAVLLFLKDKKDLGIHSEMISDGVVELVEAGVITNKKKSLHPGKIIITFLMGTKKLYDFINDNPMVEGYPVDYVNDPKVIMQNSKMVCINSCVEVDFTGQVCAESVGFKQISGVGGQVDYMRGASMADGGKSILAIPSTAAGGKISRIVPILTEGAGVTTSRYDVQYVVTEYGIALLKGKSIRERAKELIKIAHPKFREELTAQFEKRFSCKL

SEQ ID NO: 321 - protein, 433 aa, Fusobacterium sp.
MDWREDYKRKVCSPEEAVKKIKSDSRVVICHAAGEPQTVVDAMVQNYKEYKNVEIVHMVALGKCEYMKPEMKGHFRHNGLFLGGGTRDVVNAGNGDYTTSFFFEIPKLFKKGGSMPVDVALIQVSTPDEHGYCSMGITCDYTKAAAENAKCVIAQVNKFMPRVLGDNFIHISEIDVIVERDEPIIVLNPPRIGDIERKIGEYCASLINDGDTLQLGIGAIPDAVLTFLKDKKDLGIHSEMISDGVVELVKAGVINNSRKTLHKGKSIVSFLMGTRKLYDYVDDNPSVEIYPVDYVNDPRVISKNDNIVSINSAIQVDLMGQVNAETMGTKQFSGTGGQVDFVRGTAFALNGKSVIAIPSTASKGKVSRITAYLDEGAAVTTSRNDVQYIITEYGIADLKGKTLRERAKELIKIAHPDFREELTQKALEKYKTL

SEQ ID NO: 322 - protein, 434 aa, Fusobacterium sp.
MGNWKERYESKLCTSDEAIKKIAGVKRIIFEHACGESALLTEALMKNKELFKKTEIIHLVAMGKGEYAKEENSEYFRHNALFAGGTTREAANSSYGDYTPSFFFEMPKLFKKGGVLNPDVTIIQVSYPDEHGYCSYGISCDYTKCAAENSNIVIAQVNKFMPRTLGNCFIHIDSIDYIVEQATPIIELKIPVIGEIEKRIGEHCSSLIDDGATLQLGIGAIPDAVLSFLRHKKDLGIHSEMISDGVVDLVNLGVITNKRKNINVGKSIVSFLMGTRKLYDYIDNNPEIELHPVDYVNNPFIIAQNDNMISINSAIQVDLMGQVNAESIGSKQFSGTGGQVDFVRGAAMSKGGKSIIALPSTAAKGTISKIVFTLDEGAAVTTSRNDVDYIVTEYGIAHLKGKSLRERAKALIEIAHPDFREELTKKAVGKFGTL

SEQ ID NO: 323 - protein, 437 aa, Clostridium tetani
MNWEDIYKSKLLSSKEAVLKLKSENHVAVGHAASEPRLLMKALVDNKESFRDLKLYQMIALEKSECAKKGMEKHFSYNSLFVGGGSREAINDGRADYIPCFFSRIPRLFRENYIKLDAALIQVAPPDEHGFCSFGVSSDYIKPAAECAKLVIAEVNDKMPRTLGDNFIHVSDIDYIVENSREIPELKPSNIGDIERAIGENCASLINNGDVLQLGIGAIPDAVLLFLKDKKDLGIHSEMISDGVVDLVKAGVINNKKKTLHQGKIIVSFLMGTKKLYDFVDNNPMVRMYPVDYVNNPVVISKNDNMVSINSCIQIDLMGQIDAETIGFTQISGVGGQIDFIRGAGLSKNGRSIIAIPSTVGNGKISKIVPLLDKGAAVTTTRNEVDYVVTEYGIARLKGKNLRDRARELIKIAHPDFREELKLEYKNRFKEEFKRIG

SEQ ID NO: 324 - protein, 434 aa, Fusobacterium sp.
MGNWKERYESKLCTSDEAIKKIAGVKRIIFEHACGESILLTEALMKNKELFKKTEIIHLVAMGKGEYAKEENSEYFRHNALFAGGTTREAANSSYGDYTPSFFFEMPELFKKGGVLNPDVTIIQVSYPDEHGYCSYGISCDYTKCAAENSNIVIAQVNKFMPRTLGNCFIHIDNIDYIVEQATPIIELKIPVIGEIEKRIGEYCASLIDDGATLQLGIGAIPDAVLTFLRYKKDLGIHSEMISDGVVDLVNLGVITNKRKNINTGKSIVSFLMGTRKLYDYIDNNPEIELHPVEYVNNPFIIAQNDNMISINSAIQVDLMGQVNAESIGSKQFSGTGGQVDFVRGAAMSKGGKSIIALPSTAAKGTISKIVFTLDEGAAVTTSRNDVDYIITEYGIAHLKGKSLRERAKALIEIAHPDFREELRKKAVGKFGAL

SEQ ID NO: 325 - protein, 435 aa, Filifactor alocis
MDWKKIYQERLVSAEEALSHVKSGDHVALGHALAEPTYLLEEMVRLKDNYKNVKFSGLVPGKAPYAAEGLGEHFRTCSMFLGASTRKAVERGDGEFIPRFFYEVPRTYREDWPVDVAMVTLSAPDKHGFCSFGISVDFTKTIAENAKIVIGQVNKYMPRTHGDSFIHISDIDFLVEHDEPLVELPKPHIGEVESKIGEYVASLVNDGDCLQLGIGAIPDAALQFMKDKKDLGIHSEMISDGVMDLMKAGVITNKKKNFHNRRTVVTFLMGTKDLYDYVDDNPFFEMYPVDYVNDPRVVSQNDNMVCINSCIQVDLTGQVVSGSIGPRQFSGVGGQVDFVRGAAWSKGGRNIIAMPSTVKGKMSKIVPVITEGSSVTTSINDVDYIITEYGIAKLGSKSLNERARALINIAHPDFREELAKAYEERFHEKCDYKRD

SEQ ID NO: 326 - protein, 432 aa, Clostridium beijerinckii
MRNREKPFGEKIVTADEAVAAIGNGDCVVFSHAAGVPQEIPQALIRNKERFRDVHIYHMLCLGDGEYLSEGFEKHFRHITNFVGANSRKAIEQGRADFMPCFFFEIPYMFRRKQLPVDVAVIQITPPDVMGNCSFGVSCDYTKPAAEEARVVIAEMNENMPCVGGDNFIHISQIDYIVSCANPLPEILPSHIGSVEKAIGGYCAELIENGSTLQLGIGAIPDAVLMSLTDKKDLGIHTEMFTDGVIDLVEKGVINGSKKTLHPRKHIATFLMGSKRLYDFVNNNNTVELYPVDYVNNPYVIMQNADMVSINSCIEVDLMGQVVSECIGSRQFSGAGGQVDYVRGAMLSQGGKSIMAMPSTAAKGTVSRIVPFLAHGAAVTTSRNDVDYVVTEYGIAHLKAKTLKERAKSLISIAHPDFRAELIEEFNERFAQ

SEQ ID NO: 327 - protein, 433 aa, Clostridium sp.
MSFKELYQSKKMDADQALNLIRSGDRVVVGHACAEPSYLLDVLTEKAEQYENVEIVHMVAMGKAGYCAPGMEKHFRHNSLFVGGSTRKAVAEGRGDFTPCFFSEIPKLFKNDSLPVDAALIQTAPPDENGNLSLGISVDYTLQAAKSAKTVIVQVNPQMPVTFGGTIHISDVTAVVEHDAPLLTLEPPVIGDVELAIGANCAGLIRDGDTLQLGIGAIPDAVLRFLGDKKDLGIHSEMFSDGVVELAEAGVITNRLKKTNPGKYVASFLMGTRKLYDFVDGNPDVAMLPVDYVNNPVEVMKEDNIVSVNSCVQIDLMGQAASESVGLTQISGVGGQVDFVRGAAMAANGRSILAFPSTAAKGKISKIVPLLDEGAAVTTSRNDIDYAVTEYGIAKLKGRTLRQRALALIEIAHPDFRDGLRKVFEERFPGRRS

SEQ ID NO: 328 - protein, 429 aa, Porphyromonas endodontalis
MEEYKSKVVSAQEAIRAIKNGDSVVLSHAAGAPQLISRALADNYQNYQDVKIFHMLVLGDAPYCAPEMEGHFRHVTNFVGGNTRQALADGRADFIPLFFYQVPRMFENGAIPVDVAVVHVSEPNEEGYCSYGVSCDYTKPAAERAKVVIAEMNKQMPFVHGDNLIHVSKLDYIVEADYPLYEIALPTIGDVEKAIGDNVAHLVQDGDTLQLGIGAIPDAVLLFLKDKKDLGIHTEMFSDGVLELVRAGVITGKKKEIDNGKLTATFLMGSRDLYDFVNNNPDVRLAPVNWVNDPVTVMNFDRMVSINSCIEVDLMGQVASETIGYKQFSGTGGQVDYVRGASMSGHGVSIMAMPSTAAKGKVSRIVPLLAEGAAVTTSRNDVDYVVTEFGAAKLKGKSLRERAEALISIAHPDFRPQLLEEFNKRFPSK

SEQ ID NO: 329 - protein, 434 aa, Megasphaera elsdenii
MDWRKEYAEKIVSVEEAVSHVQSGNRIVFTHACGEAQALTDELVRQADRLENVEIVHMVAMGKAPYCQPGMEKHFRHNALFVGGSTRKAVEEGRGDYTPCFFHEIPKLFTDGTLPVDVAFLQLSEPDENGICSYGVSVDYTQPAAEAAKLVIAQINKNMPYTYGNGINLKDIDYIVEKDEPLIELQPPKIGETEHKIGEYVASLIHDGDTLQLGIGAIPDAVLSFLGDKKDLGIHSEMFSDGVVDLARKGVITNKKKTIDQGKFVSCFLMGTKKLYDFVNNNPDVLMERVDYTNDPFVVAKIDNIISINSAMQVDLMGQVNAEMIGYKQFSGVGGQVDFVRGASRAKGGKAIIAMPSTAGHGKISKIVPLLDEGAAVTTSRNDVDYIITEFGIAPLKGRTLRQRAIALIKIAHPDFREGLIAEFEKRFHCKFEG

SEQ ID NO: 330 - protein, 464 aa, Desulfatibacillum alkenivorans
MNWQELYNQKCVSASEAVAQIQSGDRVVVGHACGSPETLLAAMVAGKEQYTGVELVHMVSMGPSEYCSEENAVHFFHNSLFAGATSRTAVNQGRGIFTPCHFSQIPRLFTDNILPVDVTLCMISPPDEHGYCSFGISVDYTKPAAESSRLVIAEVTPHMPRTLGDSFIHVSDIDFIVETNAKPLTLPLPKITPTDEAIGGHIAELIKDGDCLQLGIGAVPDSILGFLKDKKDLGVHTEMFSDGVVDLVESGVITCAKKNLHNGKMAATFFMGTERLYKFVHNNPMVMMFPVDYTNSPAVIAQNDNMVSVNSALQVDFNGQVVSDTIGHKQYSGTGGQVDFVRGAAMSKGGRSILAFHSTAAKGKISRVVPRIDPGACVTTARTDVHYIVTEFGVADLRGKSVSERAKALINIAHPEFREDLRAEFENIYLKENPLLRPLPEEHRKRYWPHEARQDFLKQYAQAS

SEQ ID NO: 331 - protein, 438 aa, Peptoniphilus indolicus
MRRDILNWLEEYNSKLVDVHTAIENIKSGNRVVFEHACGEPKYLIDNLVENYKNLENVEIVHMVPMGSAKYCDEGMEKHFKHNGFFLGSNTKKAVHEGRADFTPSFFFEIPKLFRTKMPIDVVLVQVSKPDDFGYVSLGISVDYTKSACENAKIVIAQVNKYMPRTMGESFIHISDIDYIVEYDEPIYTLPRPNIGDVEMKIGKYCSTLIEDGDTLQLGIGAIPDAVLSFLDDKKDLGIHSEMFADGVIDLVKKGVITNKCKNLYKGKMVATFLMGTRELYDFVNDNPMVQMMPVDYVNNPCIIMQEDNLVSINSCVQVDLMGQVASESIGKNQISGVGGQVDFIRGASMAKNGRSIIAMPSTVKGKTSKIVACLDEGAAVTTSRNDVDYIVTEYGIAELKGKTLRQRSEALIRIAHPDFRDELIKAHNMKFNDELKI

SEQ ID NO: 332 - protein, 431 aa, Desulfitobacterium metallireducens
MNNWRDKYQNKIVTADQAVKNVKSGDRVVFGHACGEPEALVEALVNRGSELQDVEIVHMVAMGPGKYSQPEYAANFHHNGLFMGGPTRKALEEKRADYTPCFFSEIPRLFKDGILTVDVAFIQVTPPDEDGYCSYGISCDYTQSVAESAKIVLAQMNRNMPRTGGSRIHLDEIDFIVEQDGALIELQPPKIGDIEKAIGENVASLVQDGATLQLGIGAIPDAVLLFLNDKKDLGIHSEMFSDGVVALAEAGVVTNKRKKVHPGKFIATFLMGTRKLYDFVDNNPDVELYPVDYVNDPFIVGQNDNVVGINSALQVDLMGQVNAEMIGSRQFSGVGGQLDFVRGASRSKNGISIIALPSTASHGKISRISCELDRGAAVTTSRNDVQYIVTEYGIANLRGKNLRQRAAELIGIAHPDFRTQLFDEAKEKGIL

SEQ ID NO: 333 - protein, 430 aa, Acetonema longum
MNWRNTYRERIISAEQALKNVKSGDRVVTGHACGEPPALIEALAARAPELQNVEIVHMVAMGPAKYAQPGMEASFRHNAFFVGGSTRKAVEEKRADYTPCFFSEIPRLFRNNILPVDVALIQVTPPDEAGFCSFGISADYTVAAAECAKITIAQINNNLPRTGGVRIRLDNIQFIVEKDDPLIELKPPAIGDVEKRIGENIAGLIPDGATLQLGIGAIPDAVLLFLKEKKDLGIHSEMFSDGVVALAEAGVVTNRKKSINSGKFMVAFLMGTRKLYDFVHNNPDVELHPVDYINDPYIIGQHENMISINSALQVDCMGQVNAEMIGSRQFSGIGGQVDFVRGVSRSKGGKSIIALPATASGGRISRITCELDRGAAVSTSRNDVHYIVTEYGVANLRGKSLRERAKALIAIAHPDFRETLREEAQRTGVF

SEQ ID NO: 334 - protein, 434 aa, Porphyromonas gingivalis
  MOD_RES 4: note = Any amino acid
  MOD_RES 62: note = Any amino acid
  MOD_RES 71: note = Any amino acid
  MOD_RES 75: note = Any amino acid
  MOD_RES 112: note = Any amino acid
  MOD_RES 129: note = Any amino acid
  MOD_RES 163: note = Any amino acid
  MOD_RES 182: note = Any amino acid
  MOD_RES 243: note = Any amino acid
  MOD_RES 269: note = Any amino acid
  MOD_RES 275: note = Any amino acid
  MOD_RES 310: note = Any amino acid
  MOD_RES 322: note = Any amino acid
  MOD_RES 358: note = Any amino acid
SNAXKDVLAEYASRIVSAEEAVKHIKNGERVALSHAAGVPQSCVDALVQQADLFQNVEIYHXLCLGEGKYXAPEXAPHFRHITNFVGGNSRKAVEENRADFIPVFFYEVPSXIRKDILHIDVAIVQLSXPDENGYCSFGVSCDYSKPAAESAHLVIGEINRQXPYVHGDNLIHISKLDYIVXADYPIYSLAKPKIGEVEEAIGRNCAELIEDGATLQLGIGAIPDAALLFLKDKKDLGIHTEXFSDGVVELVRSGVITGKKKTLHPGKXVATFLXGSEDVYHFIDKNPDVELYPVDYVNDPRVIAQNDNXVSINSCIEIDLXGQVVSECIGSKQFSGTGGQVDYVRGAAWSKNGKSIXAIPSTAKNGTASRIVPIIAEGAAVTTLRNEVDYVVTEYGIAQLKGKSLRQRAEALIAIAHPDFREELTKHLRKRFG

SEQ ID NO: 335 - protein, 433 aa, Clostridium symbiosum
MSFKELYQSKKMDADQALNLIRSGNRVVVGHACAEPSYLLDVLTEKAEQYENVEIVHMVAMGKAGYCAPGMEKHFRHNSLFVGGSTRKAVAEGRGDFTPCFFSEIPKLFKNGSLPVDAALIQTAPPDENGNLSLGISVDYTLQAAKSAKTVIVQVNPQMPVTFGGTIHISDVTAVVEHDAPLLTLEPPVIGDVELAIGANCAGLIRDGDTLQLGIGAIPDAVLRFLGDKKDLGIHSEMFSDGVVELAEAGVITNRLKKTNPGKYVASFLMGTRKLYDFVDGNPDVAMLPVDYVNNPVEVMKEDNIVSVNSCVQIDLMGQAASESVGLTQISGVGGQVDFVRGAAMAANGRSILAFPSTAAKGKISKIVPLLDEGAAVTTSRNDIDYAVTEYGIAKLKGRTLRQRALALIEIAHPDFRDGLRKVFEERFPGRRS

SEQ ID NO: 336 - protein, 434 aa, Megasphaera sp.
MKDWRTKYASKIVSPAEAVSHVKSGDKIVFSHACGEAQVLTEELVNQAERLHDVEIIHMVAMGPARYCLPEMAGHFRHNALFVGATTRKAVEEGRADYTPCFFHEAPGLFREGIIPVDVVFLQVSEPDENGNCSLGISVDYTQAAAGVAKLKIAQINKNMPYTYGNGIHLDELDYIVEKDEPLIELQPPKIGEIEQCIGEHVASLIKDGDTLQLGIGAIPDAVLSFLGDKKDLGIHSEMFSDGVVDLARKGVITNRRKNIDTGKFLSCFLMGTKKLYDFIDHNPEVWIKPVDYTNDPFVIAQIENIVAINSAMQVDLMGQVNAEMIGRRQFSGVGGQVDFVRGTSRAKGGRAIIAMPSTTGKTKKISKIVAMLDAGAAVTTSRNDVDYIVTEYGIAALKGKTLRQRAEALIAIAHPDFREELKADFAKMFPVSV

SEQ ID NO: 337 - protein, 433 aa, Syntrophothermus lipocalidus
MSWKEYYEKRIVTPEEAAKLIKSNDRVVLGHACGEPKLVVDAMVERAGELENVEVVHMVAMGKARYCQPEYVKSFRHNALFVGAATRNALKEGRGDYTPCFFHEIPGLFRNNVLPVDVALVTVSPPDRHGFVSLGISVDYTRQAVLSAKTVIAEVNPNMPRTGGNSFLEVTDIDYFVLSEEPLYQLSPPEITDVERAIGENVASLIKDGDCLQLGIGAIPDAVLSFLGDKNDIGIHSEMVSDGVMNLVKKGVINCRRKTLHPNKIVITFAMGTAEFYRWLDYNPLIECYTVDYTNDPFVIARNDNMVSINSALAVDLLGQVAADTLGPYQFSGVGGQVDFVRGAARSSGGRSIIAMPATAAQGKASRIVPSLAPGQAVTTSRNDVDYVVTEFGVAKLKGKTVRDRARSLIEIAHPAFRDELREKFSQLMGYSI

SEQ ID NO: 338 - protein, 433 aa, Clostridium symbiosum
MSFKELYQSKKMDADQALNLIRSGDRVVVGHACAEPSYLLDVLTEKAEQYENVEIVHMVAMGKAGYCAPGMEKHFRHNSLFVGGSTRKAVAEGRGDFTPCFFSEIPKLLKNGSLPVDAALIQTAPPDENGNLSLGISVDYTLQAAKSAKTVIVQVNPQMPVTFGGTIHISDVTAVVEHDAPLLTLEPPVIGDVELAIGANCAGLIRDGDTLQLGIGAIPDAVLRFLGDKKDLGIHSEMFSDGVVELAEAGVITNRLKKTNPGKYVASFLMGTRKLYDFVDGNPDVAMLPVDYVNNPVEVMKEDNIVSVNSCVQIDLMGQAASESVGLTQISGVGGQVDFVRGAAMAANGRSILAFPSTAAKGKISKIVPLLDEGAAVTTSRNDIDYAVTEYGIAKLKGRTLRQRALALIEIAHPDFRDGLRKVFEERFPGRRS

SEQ ID NO: 339 - protein, 388 aa, Clostridium novyi
MARFTLPRDIYFGAGCLDVLKTIKGKRAVIVIGGGSIKRSGFLDKIEGYLKEANIETKLIEGVEPDPSVETVMNGAKVMREFNPDLIIGVGGGSPIDAAKAMWIFYEYPEFTFEKAVVPFGLPDLRQKAKFIAIPSTSGTASEVTAFSVITDYKAKIKYPLADFNLTPDIAIVDSDIAQTMPPKLVAHTGMDALTHAIEAYVAGARSAISDPLAMQAIVMVRDNLVKSFEGDKKARDEMHIAQCLAGMAFSNALLGITHSMAHKIGAVFHIPHGCANAIFLPYVIEYNRKACENRYATIARTLGLQGNTDKELVDSLIGLINELNVKLNIPSTIKEWGVSKEDFLKHVDFMAHNAVLDACTGANPREIDDKTMAKLYKCAFYGEKVDF

SEQ ID NO: 340 - protein, 394 aa, Clostridium perfringens
MGGIISMARFTLPRDIYHGKDSLEVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIVPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSYKGDMEARGEMHEAQCLAGMAFSNALLGIVHSMAHKVGAVFHIPHGCANAIFLPYVIKYNRKACENRYANIARHIGLKGESERELTDDLIDLINKFNKELNIPSSMKEYGIDENEFKANLKFIAHNAVLDPCTGSNPREINDETMEKLYTCAYYGSDVDF

SEQ ID NO: 341 - protein, 393 aa, Dorea formicigenerans
MARFTLPRDIYHGEGALEELKNLKGKKAIVVVGGGAMKRQGFLAKVEDYLKEAGMEVALFEGVEPDPSVETVMKGAKAMQEFEPDWIVAMGGGSPIDAAKAMWAFYEYPEITFEDLITPFSFPELRQKAKFAAIPSTSGTATEVTAFSVITDYAKGIKYPLADFNITPDVAIVDPALVAELPVKQVAYTGMDALTHAIEAYVSTLNCPYTDPLALKAIEMVFDYLPASYRCNMEARAKMHDAQCLAGMAFSNALLGIVHSMAHKTGAAFSTGHITHGLANAMYLPYVIKYNAKDPVAAKRYAEIARFVGLGGASEKALINSLCEKINEFNVILNIPATLKDFGIKEDEFKEKIATISENAVGDACTGSNPRTIDPATMERLFTCIYYGTEVDF

SEQ ID NO: 342 - protein, 396 aa, Clostridium hathewayi
MARFTLPRDLYHGKGALEELKNLKGNKAIVVVGGGSMKRFGFLDRVTDYLKEAGLEVKLFEGVEPDPSVDTVMKGAEMMREFGPDWIVAIGGGSPIDAAKAMWAFYEYPDTTFEELCIPFNFPTLRTKARFCAIPSTSGTATEVTAFSVITDYNKGIKYPLADFNITPDVAIVDPDLAETMPAKLTAFTGMDAMTHAIEAYVSTLHCDYTDPLALHAIKMIHNDLADSYHGDMAARDRMHNAQCLAGMAFSNALLGIVHSMAHKTGAAYSGGHIVHGCANAMYLPKVIKFNAKEAEAAKRYAEIARFIGLKGSTDGELVDALIAELRAMNGKLNIPACIREYEGGIIDEKEFNEKLPSVAELAVGDACTGSNPRPITPAEMEKLLTCCYYDKEVDF

SEQ ID NO: 343 - protein, 395 aa, Clostridium beijerinckii
MRFTLPRDLYHGKDAIEALKTLTGKKAFVVVGGGSMKRFGFLDKVVDYLKEAGMEVELFEGVEPDPSVETVMKGAKAMEVFQPDWIVAMGGGSPIDAAKAMWAFYEYPDTSFDDLIIPFNFPTLRTKAKFCAIPSTSGTATEVTAFSVITDYSKGIKYPLADFNITPDVAIVDPALAETMPKKLTAHTGMDAMTHAIEAYVSTLNCEYTDPLALHAIEMINGYLIPSYNGDMEARDKMHDAQCLAGMAFSNALLGIVHSMAHKTGAAYSGGHIVHGCANAMYLPKVIKYNSKEPEAAARYAKIARFINLEGNTDEELTDALIARIREMNKALDIPTCIRDYEGGIIDEKEFMDKLPTVAELAVGDACTGSNPRAITPAEMEKLLKCCFYDEEVTF

SEQ ID NO: 344 - protein, 388 aa, Clostridium celatum; identical to SEQ ID NO: 215

SEQ ID NO: 345 - protein, 388 aa, Clostridium saccharobutylicum
MMRFTLPRDIYYGKGSLEQLKNLKGKKAMLVLGGGSMKRFGFVDKVLGYLKEAGIEVKLIEGVEPDPSVETVFKGAELMRQFEPDWIIAMGGGSPIDAAKAMWIFYEHPEKTFDDIKDPFTVPELRNKAKFLAIPSTSGTATEVTAFSVITDYKTEIKYPLADFNITPDVAVVDSELAETMPPKLTAHTGMDALTHAIEAYVATLHSPFTDPLAMQAIEMINEHLFKSYEGDKEAREQMHYAQCLAGMAFSNALLGICHSMAHKTGAVFHIPHGCANAIYLPYVIKFNSKTSLERYAKIAKQISLAGNTNEELVDSLINLVKELNKKMQIPTTLKEYGIHEQEFKNKVDLISERAIGDACTGSNPRQLNKDEMKKIFECVYYGTEVDF

SEQ ID NO: 346 - protein, 403 aa, Ruminococcus bromii
MYRFTLPRDLYHGEDALSSLKNLSGKKAVVVVGGGSMKRFGFLDKAVDYLKEAGMEVKLIEGVEPDPSVETVMKGAAVMREFEPDWIVAMGGGSPIDAAKAMWVFYEYPNTTFEEIITPFSFPTLRTKAKFCAIPSTSGTATEVTAFSVITDYAKGIKYPLADFNITPDVAIVDPALAETMPAKLTAHTGMDAMTHAIEAYVSTLNCEYTDPLALHAIEMIHDNLKKSYEGDMACRDKMHDAQCLAGMAFSNALLGIVHSMAHKTGAAFTGGHIIHGCANAMYLPKVIKYNSKDPVAAERYAKIAKFINLKGETTEELVDALIAELRSMNDQLNIPQGIKNYGPGGVKADESIIDEKEFLEKLPEVAKNAIADACTGSNPRQPNQEEMEKLLKACYYDTEIDF

SEQ ID NO: 347 - protein, 388 aa, Clostridium botulinum
MERFTLPRDLYFGEGSLEALKTLKGKKAVVVVGGGSMKRFGFLDKVQSYLNEAGIEVKLIEGVEPDPSVETVMNGAAVMREFEPDLIVSIGGGSPIDAAKAMWIFYEYPDFTFEQAVVPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAMQAIVMVKEYLVKSYNGNKEARGQMHLAQCLAGMAFSNALLGITHSMAHKTGAVFHIPHGCANAIFLPYVIQYNSESCGERYATIAKKLGLAGENQDELVKSLIEMIREMNKTMNIPLNLKEYGITEEDFKENVKYISHNAVLDACTGSNPREINDETMEKLFACTYYGEDVTF

SEQ ID NO: 348 - protein, 388 aa, Clostridium butyricum
MARFTLPRDLYHGEGALEALKTLKGKKAFVVVGGGSMKRFGFLQKVEDYLKEAGMEVELFEGVEPDPSVETVMKGAEAMRNFEPDWIVAMGGGSPIDAAKAMWIFYEYPDFTFEQAVVPFGLPDLRQKAKFVAIPSTSGTATEVTAFSVITNYTEKIKYPLADFNLTPDVAIVDPALAQTMPAKLVAHTGMDALTHAIEAYTASLRSNFSDPLAIKAIEMVNENLVKSYEGDKVARDLMHEAQCLAGMSFSNALLGIVHSMAHKVGAVFHIPHGCANAIFLPYVIQYNRKSCEDRYADIARALKLEGNTDAELTDSLIKLINEFNNKLSIPHSMKEYGIEEAEFKANLEFIAHNAVLDACTGSNPREIDDATMAKLFECTYYGTEVNL

SEQ ID NO: 349 - protein, 388 aa, Clostridium sp.
MNRFTLPRDIYYGENSLEVLKTLEGKKAIVVTGGSSMRKNGFLDKIINFLEEAKIEVKLFEGVEPDPSIETVMKGAEMMREFAPEWIVAVGGGSPIDAAKAMWVFYEHPELTFDDIKDPFTIPALRKKAKFVAIPSTSGTATEVTAFSVITDYKKKIKYPLADFNITPDIAILDTELTMTMPKKLVAHTGMDALTHAIEAYVATNKSHFSDPLAMEAIIMIEDNILKSYEGVKEARDEMHNAQCLAGMAFSNALLGITHSMAHKTGAVFHIPHGCANAIFLPYVIQFNKKACGERYAKIARTLGLAGGNEEELINSLIDCIRQLNKAMEIPLTLKEYGVEKEEFDKYIDFISENAVVDACTASNPREISVDEMKKIFECTFDGVDVEF

SEQ ID NO: 350 - protein, 428 aa, Clostridium beijerinckii
  REGION 1..428: note = Butyrate-producing bacterium SSC/2
MMYNVFVAKQSIEIGYSNVGYIRKGSISVIQVKEMLVMARFTLPRDLYFGENALETLKTIKGNKAVLCLGGGSMKRFGFVDKVVEYLKEAGIETKLIEGIEPDPSVETVMKGAKVMQEFEPDLIIAMGGGSPIDAAKAMWVFYEHPETTFDDIKDPFTIPPLRNKATFIAIPSTSGTATEVTAFSVITDYSTGIKYPLADFEITPDVAIVDPSLAETMPQTLTAHTGMDALTHAIEAYVAGLHSPFSDPLAIHAIEMIFDNLKASYEGDMQARGNMHIAQCLAGMAFSNALLGIVHSLAHKTGAVFDTGHIPHGCANAIYLPYVIQYNAKVCEDRFAMIARKLGLPGVTDKALTLSLIKAVEDLRTSLHIVSNLKEFGVKEDEFLEKKHMIAERAVGDACTGSNPRETSVEDMEKILECIYYGKDVTF

SEQ ID NO: 351 - protein, 388 aa, Clostridium botulinum
MERFTLPRDLYFGEGSLEALKTLKGKKAVVVVGGGSMKRFGFLDKVQSYLNEAGIEVKLIEGVEPDPSVETVMNGAAVMREFEPDLIVSIGGGSPIDAAKAMWIFYEYPDFTFEQAVVPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAMQAIVMVKEYLVKSYNGNKEARGQMHLAQCLAGMAFSNALLGITHSMAHKTGAVFHIPHGCANAIFLPYVIQYNYKSCGERYATIAKKLGLAGENQDELVKSLIEMIREMNKTMNIPLNLKEYGITEEDFKENVKYISHNAVLDACTGSNPREINDEAMEKLFACTYYGEDVTF

SEQ ID NO: 352 - protein, 388 aa, Clostridium botulinum
MERFTLPRDLYFGEGSLEALKTLKGKKAVVVVGGGSMKRFGFLDKVQSYLNEAGIEVKLIEGVEPDPSVETVMNGAAVMREFEPDLIVSIGGGSPIDAAKAMWIFYEYPDFTFEQAVVPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAIQAIVMVKEYLVKSYNGNKEARGQMHLAQCLAGMAFSNALLGITHSMAHKTGAVFHIPHGCANAIFLPYVIQYNSKSCGERYATIAKKLGLAGENQDELVKSLIEMIREMNKTMNIPLNLKEYGITEEDFKENVKYISHNAVLDACTGSNPREINDETMEKLFACTYYGEDVTF

SEQ ID NO: 353 - protein, 388 aa, Clostridium kluyveri
MGRFTLPRDIYFGENALENLKNLDGNRAVIVVGGGSMKRFGFLNKVEEYLKKAGIEVKLIEGVEPDPSVDTVINGAKIMRDFKPDWIVAMGGGSPIDAAKAMWIFYEYPDFTFEKAIVPFGLPKLRGKAQFVAIPSTSGTATEVTAFSVITDYKSKIKYPLADFNLTPDAAIIDPALAETMPKKLTAHTGMDALTHAIEAYVASLHSDFSDPLAMHAIIMIHKYLLKSYEADKEARGHMHIAQCLAGMAFSNALLGITHGIAHKTGAVFHIPHGCANAIYLPYVIDFNKKVCSEKYVEIAKRLKLPGNNEDELIDSLTEMIRNMNKKMDIPLTIKEYGISEIDFNENLDFIAHNAIMDACTGSNPRKITEEEMRKLLQYMYNGRKVNF

SEQ ID NO: 354 - protein, 393 aa, Clostridium leptum
MSRFTLPRDIYYGKGSIDELKNLKGKKAIVVVGGGSMKRFGFLDKVVNNLHEAGIETMLFEGVEPDPSVETVMKGAKAMQEFQPDWIVSIGGGSPIDAAKAMWAFYEYPDTKFEDLITPFGFPTLRQKAKFLAIPSTSGTATEVTAFSVITDYHKGIKYPLADFNITPDVAIVDPDLAETMPPKLTAHTGMDALTHAIEAYVSTLNSPFTDPLALKAISMVFEYLPDSYNGNMAAREQMHYAQCLAGMAFSNALLGIVHSMAHKTGAAYSTGHIPHGCANAIYLPYVIQYNAKDATAKKRYAAIAKSVGLAGNTDDELVAALCAKIDDYNVKLNIPKTLKDFGINEAEFLEKLPEVAANAVGDACTGSNPRSITPAQMEQLLKATYYGTKVDF

SEQ ID NO: 355 - protein, 388 aa, Clostridium botulinum
MARFTLPRDLYHGEGSLETLKTLKGKKAFVVVGGGSMKRFGFLQKVEDYLKEAGMEVQLFEGVEPDPSVETVMKGAEAMRNFQPDWIVAMGGGSPIDAAKAMWIFYEYPDFTFEQAVVPFGLPDLRQKAKFVAIPSTSGTATEVTAFSVITDYKAKIKYPLADFNITPDIAIVDPELAQTMPAKLVAHTGMDALTHAIEAYTASLRSNFSDPLAIKAIEMVNENLVKSFEGDKEARNLMHEAQCLAGMAFSNALLGIVHSMAHKVGAVFHIPHGCANAIFLPYVIEYNRVECEDRYADIARALKLEGNTDAELTDSLIKLINDFNDKLNIPHSMKEYGVEEADFKANVEFIAHNAVLDACTGSSPRAIDDKTMEKLLGCTFYGTKVNF

SEQ ID NO: 356 - protein, 394 aa, Clostridium perfringens
MGGIISMARFTLPRDIYHGKDSLEVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIVPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSYKGDMEARGEMHEAQCLAGMAFSNALLGIVHSMAHKVGAVFHIPHGCANAIFLPYVIKYNRKACEDRYANIARHIGLKGESERELTDALIDLINKFNKELNIPSSMKEYGIEEDDFKANLKFIAHNAVLDPCTGSNPREIDDETMEKLYTCAYYGSDVDF

SEQ ID NO: 357 - protein, 388 aa, Clostridium perfringens
MARFTLPRDIYHGKDSLEVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIVPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSYKGNMEARGEMHEAQCLAGMAFSNALLGIVHSMAHKVGAVFHIPHGCANAIFLPYVIKYNRKACEDRYANIARHIGLKGESERELTDALIDLINKFNKELNIPSSMKEYGIEEDDFKANLKFIAHNAVLDPCTGSNPREIYDETMEKLYTCAYYGSDVDF

SEQ ID NO: 358 - protein, 386 aa, Clostridium carboxidivorans
MSRFTLPRDIYFGENTLETLKTLKGKKAIIVVGGGSMKKFGFLQKVEEYLKEAGMEIKLIEGVEPDPSVETVMKGAEIMRDFEPDWIVSIGGGSPIDAAKAMWIFYEYPEFTFEQAVVPFGIPDLRQKAKFVAIPSTSGTATEVTAFSVITDYKAKIKYPLADFNLTPDVAIIDPALAQTMPAKLTAHTGMDALTHAIEAYVAGLRSYFSDPLAMQAIVMTKDNLIKSYEGDKEARDEMHIAQCLAGMAFSNALLGITHSMAHKTGAVFHIPHGCANAIFLPYVIDFNKKTCKDRYATIAKTLGLAGNTDDELVDALTSMIQEMNKKMDIPLNLKEYGVTEEDFNENLDFIAHNAVLDACTGSNPRPITEEEIEKSIQMHIYWRES

SEQ ID NO: 359 - protein, 388 aa, Clostridium ljungdahlii
MGRFTLPRDIYFGENALENLKNLDGNKAVVVVGGGSMKRFGFLAKVEKYLKETGMEVKLIEGVEPDPSVDTVMNGAKIMRDFNPDWIVSIGGGSPIDAAKAMWIFYEYPDFTFEKAVVPFGIPKLRQKAQFVAIPSTSGTATEVTSFSVITDYKAKIKYPLADFNLTPDIAIIDPSLAETMPKKLTAHTGMDALTHAIEAYVASLHSDFSDPLAMHAITMIHKYLLKSYEEDKEARGHMHIAQCLAGMAFSNALLGITHSIAHKTGAVFHIPHGCANAIYLPYVIDFNKKACSERYAKIAKKLHLSGNSEDELIDSLTEMIRTMNKKMDIPLTIKDYGISENDFNENLDFIAHNAMMDACTGSNPRAITEEEMKKLLQYMYNGQKVNF

SEQ ID NO: 360 - protein, 395 aa, Clostridium citroniae
MRFTLPRDLYHGEHALDALKTLTGKKAFVVVGGGSMKRFGFLDKVVDYLKEAGMEVQLFEGVEPDPSVETVMKGAKAMEEFNPDWIVAMGGGSPIDAAKAMWAFYEYPDTSFEDLIIPFNFPTLRTKAKFCAIPSTSGTATEVTAFSVITDYSKGIKYPLADFNITPDVAIVDPALAETMPKKLTAHTGMDAMTHAIEAYVSTLNCEYTDPLALHAIEMINEYLIPSYNGDMEARDKMHNAQCLAGMAFSNALLGIVHSMAHKTGAAYSGGHIVHGCANAMYLPKVIKYNSKEPEAAARYAKIAKFINLPGTTDQELTDALIARIREMNKELDIPTCIRDYEGGIIDEKEFMEKLPAVAELAVGDACTGSNPRSITPAQMEKLLTCCFYDEEVTF

SEQ ID NO: 361 - protein, 388 aa, Clostridium beijerinckii; identical to SEQ ID NO: 214

SEQ ID NO: 362 - protein, 388 aa, Clostridium sporogenes
MERFTLPRDLYFGEGSLEALKTLKGKKAVVVVGGGSMKRFGFLDKVESYLNEAGIEVKLIEGVEPDPSVETVMNGAAVMREFEPDLIVSIGGGSPIDAAKAMWIFYEYPDFTFEQAVVPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAMQAIIMVKEYLVKSYNGNKGARGQMHLAQCLAGMAFSNALLGITHSMAHKTGAVFHIPHGCANAIFLPYVIQYNAKSCGERYATIAKKLGLAGENQDELVKSLIEMIREMNKTMNIPSNLKEYGIAEEEFKENVKYISHNAVLDACTGSNPREIDDETMEKLFACTYYGEDVAF

SEQ ID NO: 363 - protein, 394 aa, Clostridium perfringens; identical to SEQ ID NO: 221

SEQ ID NO: 364 - protein, 388 aa, Clostridium saccharoperbutylacetonicum; identical to SEQ ID NO: 218

SEQ ID NO: 365 - protein, 388 aa, Clostridium perfringens
MARFTLPRDIYHGKDSLEVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIVPFGLPELRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSYKGDMEARGEMHEAQCLAGMAFSNALLGIVHSMAHKVGAVFHIPHGCANAIFLPYVIKYNRKACEDRYANIARHIGLKGESERELTDALIDLINKFNKELNIPSSMKEYGIEEDDFKANLKFIAHNAVLDPCTGSNPREIDDETMEKLYTCAYYGSDVDF

SEQ ID NO: 366 - protein, 393 aa, Ruminococcus gnavus
MSRFCLPRDIYHGKGCLEELKNLKGKKAILVVGGGSMKRQGFLDRAVTYLKEAGMEVQLFEGVEPDPSVETVMKGAEAMRAFEPDWIVAMGGGSPIDAAKAMWAFYEYPDVTFEDLCIPFNFPELRQKAKFAAIPSTSGTATEVTAFSVITNYQTGVKYPLADFNITPDVAIVDPDLVAGLPVKQVAYTGMDALTHAIEAYVSTLNGPFTDPLALQAIEMVLDNLPASYNCDMDAREQMHYAQCLAGMAFSNALLGIVHSMAHKTGAAFSTGHIPHGCANAIYLPYVIKYNAKDPVAAKRYAEIARRMGLPGASEKALINSLVEKIDDFNVKLNIPKTLKDFGIEENEFKEKVAKIAELAVGDACTGSNPRAIDPATMEKLFTCTYYGTEVDF

SEQ ID NO: 367 - protein, 393 aa, Clostridium beijerinckii
MARFTLPRDIYHGKGCLEELKNLKGKKAILVVGGGSMKRQGFLDKAVNYLKEAGMEVQLFEGVEPDPSVETVMKGAEAMRAFGPDWIVAMGGGSPIDAAKAMWAFYEYPDVTFEDLCIPFNFPELRQKAKFAAIPSTSGTATEVTAFSVITDYAKGIKYPLADFNITPDVAIVDSELVEALPAHQVAYTGMDALTHAIEAYVSTLNCAYTDPLAIQAIEMVFDYLPASFKGNMEARAQMHNAQCLAGMAFSNALLGIVHSMAHKTGAAFSTGHITHGLANAMYLPYVIKYNAKDPVAAKRYAEIARRVGLAGASERALINSLCEKINEFNVILGIPATLKEFGIKEDEFKEKISEIAKNAVGDACTGSNPRAIDPETMERLFTCIYYGTEVDF

SEQ ID NO: 368 - protein, 391 aa, Clostridium beijerinckii
MARFTLPRDLYFGENALETLKTIKGNKAVLCLGGGSMKRFGFVDKVVEYLKEAGIETKLIEGIEPDPSVETVMKGAKVMQEFEPDLIIAMGGGSPIDAAKAMWVFYEHPETTFDDIKDPFTIPPLRNKATFIAIPSTSGTATEVTAFSVITDYSTGIKYPLADFEITPDVAIVDSSLAETMPQTLTAHTGMDALTHAIEAYVAGLHSPFSDPLAIHAIEMIFDNLKASYEGDMQARGNMHIAQCLAGMAFSNALLGIVHSLAHKTGAVFDTGHIPHGCANAIYLPYVIQYNAKVCEDRFAMIARKLGLPGVTDKALTLSLIKAVEDLRTSLHIVSNLKEFGVKEDEFLEKKHMIAERAVGDACTGSNPRETSVEDMEKILECIYYGKDVTF

SEQ ID NO: 369 - protein, 388 aa, Clostridium carboxidivorans
MSRFTLPRDIYFGENSLEVLKTLKGKKAVVVVGGGSMKKFGFLQKVEEYLKEAGIKIKLIEGVEPDPSVETVMNGAKVMREFQPDLIVAIGGGSPIDAAKAMWIFYEYPDFTFEKAVIPFGIPDLRQKAKFVAIASTSGTATEVTAFSVITDYKAKIKYPLADFNLTPDIAIVDPALAQTMPPKLTAHTGMDALTHAIEAYVASSRSYISDPLAIQAITMIQEFLIKSYEGDKEARGEMHVAQCLAGMAFSNALLGIAHSMAHKTGAVFHIPHGCANAIFLPYVIDFNKKSCGDRYANIAKALGFNGKTKDELIDDLTDFVKTLNKKMNIPQTLKDYGIKEADFNANLDFIAHNAMGDACTGSNPRAITEAEMKKLFQCTFTGEKVEF

SEQ ID NO: 370 - protein, 388 aa, Clostridium butyricum
MARFTLPRDIYHGEGSLEVLKTLKGKKAFVVVGGGSMKRFGFLQKVEDYLKEAGMEVELFEGVEPDPSVETVMKGAEAMRKFQPDWIVAMGGGSPIDAAKAMWIFYEYPDFTFEQAVVPFGLPELRQKAKFVAIPSTSGTATEVTAFSVITNYTEKIKYPLADFNITPDIAIVDPELAQTMPAKLVAHTGMDALTHAIEAYTASLRSNFSDPLAIKAIEMVNENLVKSFEGDKTARNLMHEAQCLAGMAFSNALLGIVHSMAHKVGAVFHIPHGCANAIFLPYVIKYNRKVCEDRYADIARALKLEGNTDAELTDSLIKLINEFNDDLSIPHSMKEYGVEEADFKSNVKFIAHNAILDACTGSSPREIDEETMEKLFECTYYGTDVNF

SEQ ID NO: 371 - protein, 388 aa, Clostridium perfringens
MARFTLPRDIYHGKDSLEVLKGLEGKKAFIVIGGGSMKRFGFLDKVLSYLKEAGMETKVFEGVEPDPSVETVMKGAKEMEEFNPDWIVSIGGGSPIDAAKAMWIFYEYPDFTFEKAIVPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSFKGDMEARGEMHEAQCLAGMAFSNALLGIVHSMAHKVGAVFHIPHGCANAIFLPYVIKYNRKACEDRYAQIARHIGLKGESERELTDALVDLINKFNKELNIPSSMKEYGIDENEFKANLKFIAHNAVLDPCTGSNPREVDDETMEKLYTCAYYGSDVDF

SEQ ID NO: 372 - protein, 388 aa, Clostridium botulinum
MERFTLPRDLYFGEGSLEALKTLKGKKAVVVVGGGSMKRFGFLDKVQSYLNEADIEVKLIEGVEPDPSVETVMNGAAVMREFEPDLIVSIGGGSPIDAAKAMWIFYEYPDFTFEQAVVPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAMQAIVMVKEYLVKSYNGNKEARGQMHLAQCLAGMAFSNALLGITHSMAHKTGAVFHIPHGCANAIFLPYVIQYNSESCGERYATIAKKLGLAGENQDELVKSLIEMIREMNKTMNIPLNLKEYGITEEDFKENVKYISHNAVLDACTGSNPREINDETMEKLFACTYYGEDVTF

SEQ ID NO: 373 - protein, 388 aa, Clostridium botulinum
MERFTLPRDLYFGKGSLEALKTLKGKKAVVVVGGGSMKRFGFLDKVQSYLNEAGIEVKLIEGVEPDPSVETVMNGAAVMREFEPDLIVSIGGGSPIDAAKAMWIFYEYPDFTFEQAVIPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAMQAIVMVKEYLVKSYNGNKEARGQMHLAQCLAGMAFSNALLGITHSMAHKTGAVFHIPHGCANAIFLPYVIQYNSKSCGERYATIAKKLGLAGENQDELVKSLIEMIREMNKTMNIPLNLKEYGIAEEDFKENVKYISHNAVLDACTGSNPREINDETMEKLFACTYYGEDVTF

SEQ ID NO: 374 - protein, 393 aa, Clostridium beijerinckii
MSRFTLPRDIYHGKGCLEELKNLKGKRAILVVGGGSMKRQGFLDKAVNYLKEAGMEVQLFEGVEPDPSVETVMKGAEAMRAFEPDWIVAMGGGSPIDAAKAMWAFYEYPDVTFEDLCIPFNFPELRQKAKFAAIPSTSGTATEVTAFSVITDYAKGIKYPLADFNITPDVAIVDSELVEGLPAHQVAYTGMDALTHAIEAYVSTLNCAYTDPLAIQAIEMVFDYLPASFRGNMTARAKMHDAQCLAGMAFSNALLGIVHSMAHKTGAAFSTGHITHGLANAMYLPYVIKYNAKDPVAAKRYAEIARRVGLEGSSEQSLINSLCEKINEFNVILGIPATLKEFGIKEDEFKEKVSAVAENAVGDACTGSNPRAIDPATMERLFTCIYYGTEVDF

SEQ ID NO: 375 - protein, 393 aa, Flavonifractor plautii
MSRFTLPRDIYYGANALENLKNLKGSRVVLVLGGGSMKKFGFVDKALGYLKEAGIETRLIENVEPDPSVETVMSGAAVMREFEPDWIISMGGGSPIDAAKAMWAFYEYPETTFEDLITPFSFPELRQKARFCAIPSTSGTATEVTAFSVITDYQKGIKYPLADFNITPDVAIIDPALAETMPPKLVAHTGMDALTHAIEAYVSTLHTVFTDPLALKAIHIVEKDLLRSFNGDMGCREEMHYGQCLAGMAFSNALLGIVHSMAHKTGAAFSTGHITHGLANAMYLPYVIAYNAKADGVAERYADIARTMGVVGDTTAELVEGLRAKIRSMNDAMGIPNTLKDFGIIEDEFKEKLDAIAANAVGDACTGSNPRPIDPATMAKLFTCIYYGTEVDF

SEQ ID NO: 376 - protein, 395 aa, Clostridium bolteae
MRFTLPRDLYHGKDALEALKTLAGKKAIVVVGGGSMKRFGFLDKVVDYLKEAGMEVQLFEGVEPDPSVDTVLKGAAAMQEFEPDWIVAVGGGSPIDAAKAMWAFYEYPDTSFEDLITPFSFPTLRTKAKFCAISSTSGTATEVTAFSVITDYKKGIKYPLADFNITPDVAIVDPALAETMPKKLTAHTGMDAMTHAIEAYVSTLHCEYTDPLALHAIEMINEYLIPSYNGDMEARDKMHDAQCLAGMAFSNALLGIVHSMAHKTGAAYSGGHIVHGCANAMYLPKVIKFNSKEPEAAARYAKIAKFINLPGTTDEELTDALIARLLEMNKALDIPACIKDYEGGIIDEKEFMDKLPKVAELAVGDACTGSNPRTITPEVMEQLLKCCYYDEEVDF

SEQ ID NO: 377 - protein, 397 aa, Clostridium kluyveri
MNSIMRRKVMGRFTLPRDIYFGENALENLKNLDGNRAVIVVGGGSMKRFGFLNKVEEYLKKAGIEVKLIEGVEPDPSVDTVINGAKIMRDFKPDWIVAMGGGSPIDAAKAMWIFYEYPDFTFEKAIVPFGLPKLRGKAQFVAIPSTSGTATEVTAFSVITDYKSKIKYPLADFNLTPDAAIIDPALAETMPKKLTAHTGMDALTHAIEAYVASLHSDFSDPLAMHAIIMIHKYLLKSYEADKEARGHMHIAQCLAGMAFSNALLGITHGIAHKTGAVFHIPHGCANAIYLPYVIDFNKKVCSEKYVEIAKRLKLPGNNEDELIDSLTEMIRNMNKKMDIPLTIKEYGISEIDFNENLDFIAHNAIMDACTGSNPRKITEEEMRKLLQYMYNGRKVNF

SEQ ID NO: 378 - protein, 394 aa, Clostridium perfringens
MGGIISMARFTLPRDIYHGKDSLEVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIVPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSFKGDMEARGEMHEAQCLAGMAFSNALLGIVHSMAHKVGAVFHIPHGCANAIFLPYVIKYNRKACEDRYANIARHIGLKGESERELTDALIDLINKFNKELNIPSSMKEYGIDENEFKTNLKFIAHNAVLDPCTGSNPREVDDETMEKLYTCAYYGSDVDF

SEQ ID NO: 379 - protein, 388 aa, Clostridium sp.
MARFTLPRDLYHGEGALEVLKTLKGKKAFVVVGGGSMKRFGFLKQVEDYLTEAGMEVELFEGVEPDPSVETVMKGAEAMRNFEPDWIVAMGGGSPIDAAKAMWIFYEYPEFTFEQAVVPFGLPELRQKAKFVAIPSTSGTATEVTAFSVITNYTEKIKYPLADFNITPDIAIVDPVLAQTMPKTLTAHTGMDALTHAIEAYTASLRSNFSDPLAIKALQMVQQNLLKSFEGDKEARNLMHEAQCLAGMAFSNALLGIVHSMAHKVGAVFHIPHGCANAIFLPYVIQYNRTKCEDRYADIARALKLEGSTDSELTDSLIGMINKMNSDLNIPHSMKEYGVTEDDFKANLSFIAHNAVLDACTGSNPREIDDATMEKLFECTYYGTKVEL

SEQ ID NO: 380 - protein, 388 aa, Clostridium colicanis
MARFTLPRDLYHGKGSLEVLKSIKGKKAFVVVGGGSMKRFGFLDKVESYLKEAGIEVEIFEGVEPDPSVETVMKGAEAMRKFQPDWIVAMGGGSPIDAAKAMWIFYEYPDFTFEKAVVPFGLPELRQKAKFIAIPSTSGTATEVTAFSVITNYTEKIKYPLADFNLTPDIAIVDPDLAQTMPKKLVAHTGMDALTHGIEAYTASLRSNFSDPLAAKGIKMVVENLLKSFEGGEKAREEMHDAQCLVGMAFSNALLGIVHSMAHKTGAVFHIPHGCANAIFLPYVIKYNRKNCEDRYADIARMLKLEGNTDSELTDSLINLINEMNTNLEIPHTMKEYGVEEEDFKDNLEFISKNAVLDACTGSNPREIDDKTMEKLFTCAYYGTEVDF

SEQ ID NO: 381 - protein, 388 aa, Clostridium botulinum
MERFTLPRDLYFGEGSLEALKTLKGKKAVVVVGGGSMKRFGFLDKVQSYLNEAGIEVKLIEGVEPDPSVETVMNGAAVMREFEPDLIVSIGGGSPIDAAKAMWIFYEYPEFTFEQAVIPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAMQAIVMVKEYLVKSYNGNKEARGQMHLAQCLAGMAFSNALLGITHSMAHKTGAVFHIPHGCANAIFLPYVIQYNSKSCGERYATIAKKLGLAGENQDELVKSLIEMIREMNKTMNIPSNLKEYGITEEDFKENVKYISHNAVLDACTGSNPREINDETMEKLFACTYYGEEVTF

SEQ ID NO: 382 - protein, 388 aa, Clostridium ljungdahlii
MARFTLPRDIYFGENSLETLKDLDGKKAVIVVGGGSMKRFGFLDKVVNYLKEAGIESKLIEGVEPDPSVETVMNGAKLMREYEPDLIVSIGGGSPIDAAKAMWIFYEYPEFTFKEAVVPFGLPKLRQKATFIAIPSTSGTATEVTAFSVITDYKAKIKYPLADFNLTPDIAIIDPALAQTMPPKLTAHTGMDALTHAIEAYVAGLHSVFSDPLAIQAIVMVNQYLIKSYNEDKEARNQMHLAQCLAGMAFSNALLGITHSLAHKTGAVFHIPHGCANAIYLPYVIDFNKKACAPRYAEIARSLKLPGNTDDELVDSLTNMIKDMNKSMDIPLTLKDYGVDEKEFKDSEDFIAHNAVLDACTGSNPRSINDTEMKKLLEYIYYGKKVDF

SEQ ID NO: 383 - protein, 388 aa, Clostridium cellulovorans
MNRFTLPRDLYFGKGSMEQLKNLKGKKAVIVVGGGSMKKFGFLDKAIGYLQEAGIETRLIEGVEPDPSVETVFKGAEAMREFEPDWIISIGGGSPIDAAKAMWVFYEHPELTFDDIKDPFTIPELRNKAKFVAIPSTSGTATEVTAFSVITDYKTEIKYPLADFNITPDIAIVDSELAETMPKTLTAHTGMDALTHAIEAYVAGLHSDFSDPLAIKAIEMIYDELLKSYNEDKDAREKMHYAQCLAGMAFSNALLGITHSMAHKTGAVFHIPHGCANAIYLPYVIEFNKKVAAERYADIAKRIGLKGETTDELVDALVKSIRDLNVKMDIPLTLKDYGIDEALFKEKVALISERAIGDACTGSNPRTITVTEMEKLFNCTYYGIKVDF

SEQ ID NO: 384 - protein, 393 aa, Clostridium beijerinckii
MSRFCLPRDIYHGKGCLEELKNLKGKKAILVVGGGSMKRQGFLDRAVGYLKEAGMEVQLFEGVEPDPSVETVMKGAEAMRAFEPDWIVSMGGGSPIDAAKAMWAFYEYPDTTFEELCTPFNFPELRQKAKFCAIPSTSGTATEVTAFSVITNYQTGVKYPLADFNITPDVAIVDPDLVAGLPVKQVAYTGMDALTHAIEAYVSTLNGPFTDPLALQAIEMVLDYLPASYNCNMDAREQMHYAQCLAGMAFSNALLGIVHSMAHKTGAAFSTGHIPHGCANAIYLPYVIQYNAKDSVAAKRYAEIARRMGLEGNSEKALINSLVAKIDEFNAKLNIPKTLKDFGINEEEFKEKIAKISELAVGDACTGSNPRAIDPAAMEKLFTCTYYGTEVDF

SEQ ID NO: 385 - protein, 393 aa, Clostridium sp.
MSRFTLPRDVYHGKGCLEELKNLRGKKAIIVVGGGSMKRQGFLDRAVGYLKEAGMEVELFEGVEPDPSVETVMKGAEAMRAFEPDWIVSMGGGSPIDAAKAMWAFYEYPDVTFEDLCIPFNFPELRQKAKFCAIPSTSGTATEVTAFSVITNYSTGVKYPLADFNITPDVAIVDPDLVTGLPVKQVAYTGMDALTHAVEAYVSTLNGPFTDPLALQAIEMVLDYLPASYHCNMDAREQMHYAQCLAGMAFSNALLGIVHSMAHKTGAAFSTGHIPHGCANAIYLPYVIKYNAKEPTAASRYAEIARRMGLEGTSEKALINSLCAKIDAFNVELNIPKTLKDFGINEDEFKEKVAKIAELAVGDACTGSNPRAIDPAAMEQLLTCTYYGTEVDF

SEQ ID NO: 386 - protein, 388 aa, Clostridium butyricum
MARFTLPRDIYHGEGSLEVLKTLKGKKAFVVVGGGSMKRFGFLQKVENYLKEAGMEVELFEGVEPDPSVETVMKGAEAMRKFQPDWIVAMGGGSPIDAAKAMWIFYEYPDFTFEQAVVPFGLPELRQKAKFVAIPSTSGTATEVTAFSVITNYTEKIKYPLADFNITPDIAIVDPELAQTMPAKLVAHTGMDALTHAIEAYTASLRSNFSDPLAIKAIEMVSENLVKSFEGDKTARNLMHEAQCLAGMAFSNALLGIVHSMAHKVGAVFHIPHGCANAIFLPYVIKYNRKVCEDRYADIARALKLEGNTDSELTDSLIKLINEFNDDLSIPHSMKEYGVEEADFKSNVKFIAHNAILDACTGSSPREIDEETMEKLFECTYYGTDVNF

SEQ ID NO: 387 - protein, 388 aa, Clostridium botulinum
MARFTLPRDLYHGEGSLETLKTLKGKKAFVVVGGGSMKRFGFLQKVEDYLKEAGMEVQLFEGVEPDPSVETVMKGAEAMRNFEPDWIVAMGGGSPIDAAKAMWIFYEYPDFTFEQAVVPFGLPDLRQKAKFVAIPSTSGTATEVTAFSVITDYKAKIKYPLADFNITPDIAIVDPELAQTMPAKLVAHTGMDALTHAIEAYTASLRSNFSDPLAIKAIEMVNEHLVKSFEGDKEARNLMHEAQCLAGMAFSNALLGIVHSMAHKVGAVFHIPHGCANAIFLPYVIQYNRIECEDRYGDIARALKLEGNTDAELTDSLIKLINDFNDALNIPHSMKEYGVEEADFKANVEFIAHNAVLDACTGSSPRAIDDKTMEKLLGCTFYGTKVNF

SEQ ID NO: 388 - protein, 328 aa, Clostridium botulinum
MERFTLPRDLYFGEGSLEALKTLKGKKAVVVVGGGSMKRFGFLDKVQSYLNEAGIEVKLIEGVEPDPSVETVMNGAAVMREFEPDLIVSIGGGSPIDAAKAMWIFYEYPDFTFEQAVVPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAMQAIVMVKEYLVKSYNGNKEARGQMHLAQCLAGMAFSNALLGITHSMAHKTGAVFHIPHGCANAIFLPYVIQYNSESCGERYATIAKKLGLAGENQDELVKSLIEMIREMNKTM

SEQ ID NO: 389 - protein, 393 aa, Ruminococcus torques
MSRFCLPRDIYHGKGCLEELKNLKGKKAILVVGGGSMKRQGFLDKAVNYLKEGGMEVQLFEGVEPDPSVETVMKGAEAMRAFEPDWIVAMGGGSPIDAAKAMWAFYEYPEISFEDLITPFSFPELRQKAKFAAIPTTSGTATEVTAFSVITNYQTGVKYPLADFNITPDVAIVDPDLVAGLPVKQVAYTGMDALTHAIEAYVSTLNGPFTDPLALQAIEMVLDYLPGSYKCDMVAREQMHYAQCLAGMAFSNALLGIVHSMAHKTGAAFSTGHIPHGCANAIYLPYVIKYNAKDAVAAKRYAEIARRMGLAGTSEQALINSLVEKIDEFNVRLNIPKTLKDFGINEEEFKEKVDKIAELAVGDACTGSNPRAIDPAAMAKLLTCTYYGTEVDF

SEQ ID NO: 390 - protein, 388 aa, Clostridium carboxidivorans; identical to SEQ ID NO: 216

SEQ ID NO: 391 - protein, 388 aa, Clostridium beijerinckii
MARFTLPRDLYHGEGALEVLKTLKGKKAFVVVGGGSMKRFGFLKQVEDYLTEAGMEVELFEGVEPDPSVETVMKGAEAMRNFEPDWIVAMGGGSPIDAAKAMWIFYEYPDFTFEQAVVPFGLPELRQKAKFVAIPSTSGTATEVTAFSVITNYTEKIKYPLADFNITPDIAIVDPVLAQTMPKTLTAHTGMDALTHAIEAYTASLRSNFSDPLAIKALQMVQENLIKSFEGDKEARNLMHEAQCLAGMAFSNALLGIVHSMAHKVGAVFHIPHGCANAIFLPYVIQYNRTKCEDRYGDIARALKLKGNNDAELTDSLIELINGLNDKLEIPHSMKAYGVTEEDFKANLSFIAHNAVLDACTGSNPREIDDATMEKLFECTYYGTKVNL

SEQ ID NO: 392 - protein, 388 aa, Clostridium carboxidivorans
MSRFTLPRDLYFGDNSLAELKNLKGKKAVVVVGGGSMKKFGFLEKVENYLKEAGMEVKLIEGVEPDPSVETVFNGAQVMRDFEPDWIVSIGGGSPIDAAKAMWIFYEYPDFTFEQAVIPFGIPELRQKARFVAIPSTSGTATEVTAFSVITDYKARIKYPLADFNLTPDVAIIDPALAQTMPPKLTAHTGMDALTHAIEAYVASARSHFSDPLAMQAIVMVREHLIKSYSGDKEARNEMHIAQCLAGMAFSNALLGIVHSMAHKSGAVFHIPHGCANAIYLPYVIQFNRKASESRFAVIAKTLGLDGNTDADLTDSLVAMIRDMNKQLDIPLTLKDYGITEEDFNENVDFISKNALTDACTGSNPREIDFDQMKKLFQCTFTGEDCTF

SEQ ID NO: 393 - protein, 388 aa, Clostridium sp.
MMRFTLPRDIYYGKGCLEQLKTLKGQRAVLALGGGSMKKFGFVDKALEYLKEAGIEVKLIEGIEPDPSVETVLKGAEVMRDFKPDWIIAMGGGSPIDAAKAMWVFYEHPEKTFDDIKDPFTIPELRSKAKFLAIPSTSGTATEVTAFSVITDYKTEIKYPLADFNITPDVAIVDSELAETMPAKLTAHTGMDALTHAIEAYVATLHSPFTDPLALQAIEMINEYLLKSYDGDKDAREQMHYAQCLAGMAFSNALLGITHSMAHKTGAVFHIPHGCANAIYLPYVIDFNKKAALDRYAKIAKRLGLSGTSDDELVDSLTSLIKELNKKMAIPSTLKEYGIDEEEFKAKVDLISERAIGDACTGSNPRQINKDEMKKIFECVYYGNKVEF

SEQ ID NO: 394 - protein, 396 aa, Ruminococcus torques
MSEMSRFCLPRDIYHGKGCLEELKNLKGKKAILVVGGGSMKRQGFLDRAVGYLKEAGMEVQLFEGVEPDPSVETVMKGAEAMRAFEPDWIVSMGGGSPIDAAKAMWAFYEYPDTTFEELCTPFNFPELRQKAKFCAIPSTSGTATEVTAFSVITNYQTGVKYPLADFNITPDVAIVDPDLVAGLPVKQVAYTGMDALTHAIEAYVSTLNGPFTDPLALQAIEMVLDYLPASYNCNMDAREQMHYAQCLAGMAFSNALLGIVHSMAHKTGAAFSTGHIPHGCANAIYLPYVIQYNAKDSVAAKRYAEIARRMGLEGNSEKALINSLVAKIDEFNAKLNIPKTLKDFGINEEEFKEKIAKISELAVGDACTGSNPRAIDPAAMEKLFTCTYYGTEVDF

SEQ ID NO: 395 - protein, 396 aa, Clostridium saccharolyticum
MARFTLPRDLYHGKGALEELKNIKGKKAIVVVGGGSMKRFGFLDRVEACLKEAGMEVKLFEGVEPDPSVDTVMKGAEIMREFEPDWIVAIGGGSPIDAAKAMWAFYEYPETSFEDLCIPFNFPTLRTKARFCAIPSTSGTATEVTAFSVITDYKKGIKYPLADFNITPDIAIVDPDLAETMPQKLTAHTGMDAMTHAVEAYVSTLHCDYTDPLALHAIKMIHNDLIDSFNGDKEARARMHNAQCLAGMAFSNALLGIVHSMAHKTGAAYSGGHIVHGCANAMYLPKVIKFNSKAPEAAKRYAEIARFIGLQGGDENELVDALITEIRYMNKKLDIPTCIKEYEGGIIDEAEFNEKLVSVAELAVGDACTGSNPRPITPAEMEKLLTCCYYDKEVDF

SEQ ID NO: 396 - protein, 388 aa, Clostridium ljungdahlii
MERFTLPRDIYFGEDALGALKTLKGKKAVVVVGGGSMKRFGFLDKVEEYLKEANIEVKLIEGVEPDPSVETVMKGAKIMTEFGPDWIVAIGGGSPIDAAKAMWLFYEYPDFTFKQAIVPFGLPELRQKAKFVAIASTSGTATEVTSFSVITDYKAKIKYPLADFNLTPDIAIVDPALAQTMPPKLTAHTGMDALTHALEAYVASARSDISDPLAIHSIIMTRDNLLKSYKGDKDARNKMHISQCLAGMAFSNALLGITHSLAHKTGAVWHIPHGCANAIYLPYVLDFNKKACSDRYANIAKILGLKGTTEDELVDSLVKMVQDMDKELNIPLTLKDYGISKDDFNSNVDFIAKNALLDACTGANPRPIDFDQMKKILQCIYDGKKVTF

SEQ ID NO: 397 - protein, 388 aa, Clostridium butyricum
MARFTLPRDLYHGEGALEALKTLKGKKAFVVVGGGSMKRFGFLQKVEDYLKEAGMEVELFEGVEPDPSVETVMKGAEAMRNFEPDWIVAMGGGSPIDAAKAMWIFYEYPDFTFEQAVVPFGLPDLRQKAKFVAIPSTSGTATEVTAFSVITNYTEKIKYPLADFNLTPDVAIVDPALAQTMPAKLVAHTGMDALTHAIEAYTASLRSNFSDPLAIKAIEMVNENLVKSFEGDKVARDLMHEAQCLAGMSFSNALLGIVHSMAHKVGAVFHIPHGCANAIFLPYVIQYNRKSCEDRYADIARALKLEGNTDAELTDSLIKLINEFNDKLSIPHSMKEYGVEEAEFKANLEFISHNAVVDACTGSNPREIDDATMAKLFECTYYGTEVNL

SEQ ID NO: 398 - protein, 388 aa, Clostridium perfringens
MARFTLPRDIYHGKDSLKVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIVPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSYKGDMEARGEMHEAQCLAGMAFSNALLGIVHSMAHKVGAVFHIPHGCANAIFLPYVIKYNRKACEDRYANIARHIGLKGESERELTDALIDLINKFNKELNIPSSMKEYGIEEDDFKANLKFIAHNAVLDPCTGSNPREIDDETMEKLYTCAYYGSDVDF

SEQ ID NO: 399 - protein, 388 aa, Clostridium perfringens
MARFTLPRDIYHGKDSLEVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIIPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSYKGDMEARGEMHEAQCLAGMAFSNALLGIVHSMAHKVGAVFHIPHGCANAIFLPYVIKYNRKACENRYANIARHIGLKGESERELTDDLIDLINKFNKELNIPSSMKEYGIDENEFKANLKFIAHNAVLDPCTGSNPREINDETMEKLYTCAYYGSDVDF

SEQ ID NO: 400 - protein, 388 aa, Clostridium beijerinckii; identical to SEQ ID NO: 219

SEQ ID NO: 401 - protein, 388 aa, Clostridium saccharoperbutylacetonicum
MMRFTLPRDIYYGKGSLEQLKTLKGKKAVLVLGGGSMKKFGFVDKALEYLKEAGIEVKLIEGVEPDPSVETVFKGAETMREFGPDWIISMGGGSPIDAAKAMWIFYEYPDFTFEKAVVPFGLPELRQKAKFIAIPSTSGTATEVTAFSVITDYKTEIKYPLADFNITPDIAIVDSELAETMPATLTAHTGMDALTHAIEAYVATLHSPFTDPLALQAIEMIRDFLLKSYEGDKDAREQMHYSQCLAGMAFSNALLGITHSMAHKTGAVFHIPHGCANAIYLPYVIDFNKKTALDRYAYIAKRLGLSGTTDDELVDSLTNLIKEFNKKMSIPLTLKEYGIDEEVFKAKVDMISERAIADACTGSNPRQLNKDEMKKIFECIYYGNKVDF

SEQ ID NO: 402 - protein, 393 aa, Clostridium beijerinckii; identical to SEQ ID NO: 220

SEQ ID NO: 403 - protein, 446 aa, Ruminococcus lactaris
MQKEVLCKVTIRQLCFTWNLYKISKNRVAFLRAERYNIPVKKITILHKKERFVMSRFCLPRDIYHGKGCLEELKNLKGKKACLVVGGGSMKRQGFLDRAVNYLKEGGMEVKLIEGVEPDPSVETVMKGAEVMREFEPDWIVAMGGGSPIDAAKAMWVFYEYPEVSFEDIITPFSFPELRQKAKFAAIPSTSGTATEVTAFSVITNYQTGVKYPLADFNITPDVAIVDPDLVSGLPVKQVAYTGMDALTHAIEAYVSTLNGPFTDPLALQAIEMVLDYLPASYNCDMVAREQMHYAQCLAGMAFSNALLGIVHSMAHKTGAAFSTGHIPHGCANAIYLPYVIQYNAKDPVAAKRYAEIARRMGLPGTSEKALINSLIEKIDDFNVRLNIPKTLKDFGINEDEFKEKVDKIAELAVGDACTGSNPRAIDPAAMAKLLTCTYYGTEVDF

SEQ ID NO: 404 - protein, 395 aa, Clostridium clostridioforme
MRFTLPRDLYHGKDALEALKTLTGKKAIVVVGGGSMKRFGFLDKVVDYLKEAGMEVKLFEGVEPDPSVDTVLKGAAAMQEFEPDWIVAVGGGSPIDAAKAMWAFYEYPDTSFEDLITPFSFPTLRTKAKFCAISSTSGTATEVTAFSVITDYKKGIKYPLADFNITPDVAIVDPALAETMPGKLTAHTGMDAMTHAIEAYVSTLHCEYTDPLALHAIEMINEYLIPSYNGDMEARDKMHDAQCLAGMAFSNALLGIVHSMAHKTGAAYSGGHIVHGCANAMYLPKVIKFNSKEPEAAARYAKIAKFISLPGTTEEELTDALIARLLEMNKALDIPACIKDYEGGIIDEKEFMDKLPKVAELAVGDACTGSNPRSITPRVMEQLLKCCYYDEEVDF

SEQ ID NO: 405 - protein, 393 aa, Clostridium nexile
MSRFCLPRDIYHGKGCLEELKNLKGKKAILVVGGGSMKRQGFLDKAVNYLKEAGMEVQLFEGVEPDPSVETVMKGAEAMRAFEPDWIVAMGGGSPIDAAKAMWAFYEYPDVTFEDLCIPFNFPELRQKAKFAAIPSTSGTATEVTAFSVITDYAKGIKYPLADFNITPDVAIVDSELVEGLPAHQVAYTGMDALTHAIEAYVSTLNCAYTDPLAIQAIEMVFDYLPASYKGNMDARAHMHDAQCLAGMAFSNALLGIVHSMAHKTGAAFSTGHITHGLANAMYLPYVIKYNAKDPVAAKRYAEIARRVGLEGASEKALINSLCKKIDDFNVILGIPATLKEFGIKEDEFKEKIASIAELAVGDACTGSNPRAIDPATMERLFTCVYYGTEVDF

SEQ ID NO: 406 - protein, 389 aa, Clostridium tetani; identical to SEQ ID NO: 211

SEQ ID NO: 407 - protein, 388 aa, Clostridium sporogenes
MERFTLPRDLYFGEGSLETLKTLKGKKAVVVVGGGSMKRFGFLDKVESYLNEAGIEVKLIEGVEPDPSVETVMNGAAVMREFEPDLIVSIGGGSPIDAAKAMWIFYEYPDFTFEQAVVPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAMKAIVMVKEYLVKSYNGDKEARGQMHLAQCLAGMAFSNALLGITHSMAHKTGAVFHIPHGCANAIFLPYVIQYNAKSCGERYATIAKNLGLAGENQDELVKALIEMIREMNKTMNIPLNLKEYGITEEEFKENVKYISHNSVLDACTGSNPREIDDETMEKLFACTYYGQDVEF

SEQ ID NO: 408 - protein, 388 aa, Clostridium beijerinckii
MARFTLPRDLYHGEGALEVLKTLKGKKAFVVVGGGSMKRFGFLKQVEDYLTEAGMEVELFEGVEPDPSVETVMKGAEAMRNFEPDWIVAMGGGSPIDAAKAMWIFYEYPDFTFEQAVVPFGLPELRQKAKFVAIPSTSGTATEVTAFSVITNYTEKIKYPLADFNITPDIAIVDPVLAQTMPKTLTAHTGMDALTHAIEAYTASLRSNFSDPLAIKALQMVQENLIKSFEGDKEARNLMHEAQCLAGMAFSNALLGIVHSMAHKVGAVFHIPHGCANAIFLPYVIQYNRTKCEDRYADIARALKLEGNTDSELTDSLIGMINKMNSDLNIPHSMKEYGVTEEDFKANLSFIAHNAVLDACTGSNPREIDDATMEKLFECTYYGTKVEL

SEQ ID NO: 409 - protein, 388 aa, Clostridium botulinum
MARFTLPRDLYHGEGSLETLKTLKGKKAFVVVGGGSMKRFGFLQKVEDYLKEAGMEVQLFEGVEPDPSVETVMKGAEAMRNFEPDWIVAMGGGSPIDAAKAMWIFYEYPDFTFEQAVVPFGLPDLRQKAKFVAIPSTSGTATEVTAFSVITDYKAKIKYPLADFNITPDIAIVDPELAQTMPAKLVAHTGMDALTHAIEAYTASLRSNFSDPLAIKAIEMVNENLVKSFEGDKEARNLMHEAQCLAGMAFSNALLGIVHSMAHKVGAVFHIPHGCANAIFLPYVIEYNRAECEDRYADIARALKLEGNTDAELTDSLIKLINDFNDKLNIPHSMKEYGVEEADFKANVEFIAHNAVLDACTGSSPRAIDYKTMEKLLGCTFYGTKVNF

SEQ ID NO: 410 - protein, 388 aa, Clostridium botulinum
MERFTLPRDLYFGEGSLEALKTLKGKKAVVVVGGGSMKRFGFLDKVQSYLNEAGIEVKLIEGVEPDPSVETVMNGAAVMREFEPDLIVSIGGGSPIDAAKAMWIFYEYPDFTFEQAVVPFGIPELRQKARFVAIPSTSGTATEVTAFSVITDYKKKIKYPLADFNLTPDIAIVDPDLAQTMPAKLTAHTGMDALTHAIEAYVAGLRSVFSDPLAMQAIVMVKEYLVKSYNGNKEARGQMHLAQCLAGMAFSNALLGITHSMAHKTGAVFHIPHGCANAIFLPYVIQYNFKSCGERYATIAKKLGLAGENQDELVKSLIEMIREMNKTMNIPLNLKEYGITEEDFKENVKYISHNAVLDACTGSNPREINDETMEKLFACTYYGEDVTF

SEQ ID NO: 411 - protein, 393 aa, Clostridium beijerinckii
  REGION 1..393: note = Butyrate-producing bacterium SS3/4
MARFTLPRDIYHGKGCLEELKNLKGKKAFLVVGGGSMKRQGFLDKAVHYLKDGGMEVEVFEGVEPDPSVETVMRGAEAMRKFEPDWIVSMGGGSPIDAAKAMWAFYEYPETTFEDLITPFSFPELRQKARFAAIPSTSGTATEVTAFSVITNYQTGVKYPLADFNITPDVAIVDPDLVMGLPVKQVAYTGMDALTHAIEAYVSTLNGPFTDPLALQAIEMVLEYLPASYNGNTHAREEMHYAQCLAGMAFSNALLGIVHSMAHKTGAAFSTGHIPHGCANAIYLPYVIQYNAKDTVAAERYAEIARRMGLEGRSRQALINSLCAKINEFNEKLNIPKTLKEFGINEDEFKEKVDRIAELAVGDACTGSNPREIDPANMAKLLTCTYYGTEVDF

SEQ ID NO: 412 - protein, 391 aa, Anaerostipes caccae; identical to SEQ ID NO: 213

SEQ ID NO: 413 - protein, 427 aa, Clostridium sp.
MKASAQTEACLQWGMRMSGDGPAGRVHPFMKEESEMARFTLPRDIYHGKGCLEELKNLKGKRAILVVGGGSMKRQGFLDKAVEYLKEAGMEVRVFEGVEPDPSVETVMKGAQVMREFEPDWIVSMGGGSPIDAAKAMWAFYEYPEVTFEELCTPFNFPELRTKAKFAAIPSTSGTATEVTAFSVITDYEKGIKYPLADFNITPDVAIVDPELVEGLPAKQVAYTGMDALTHAIEAYVSTLHCTFTDPLAIKAIQIVNSDLQKSYDGDMRSREEMHYGQCLAGMAFSNALLGIVHSMAHKTGAAFSTGHITHGLANAMYLPYVISYNAKEPEAAARYAEIANAIGITGTVEECIEGLRKKIRSMNDYMGIPNTLKEFGIEEKEFKEKLAAISENAVGDACTGSNPRTIDPKTMEKLFTCIYYGTEVDF

SEQ ID NO: 414 - protein, 411 aa, Clostridium sp.
  REGION 1..411: note = [Clostridium] cf. saccharolyticum K10
MSGDGPAGRVHPFMKEESEMARFTLPRDIYHGKGCLEELKNLKGKRAILVVGGGSMKRQGFLDKAVEYLKEAGMEVRVFEGVEPDPSVETVMKGAQVMREFEPDWIVSMGGGSPIDAAKAMWAFYEYPEVTFEELCTPFNFPELRTKAKFAAIPSTSGTATEVTAFSVITDYEKGIKYPLADFNITPDVAIVDPELVEGLPAKQVSYTGMDALTHAIEAYVSTLHCTFTDPLAIKAIQIVNSDLQKSYDGDMRSREEMHYGQCLAGMAFSNALLGIVHSMAHKTGAAFSTGHITHGLANAMYLPYVISYNAKEPEAAARYAEIANAIGITGTVEECIEGLRKKIRSMNDYMGIPNTLKEFGIEEKEFKEKLAAISENAVGDACTGSNPRTIDPKTMEKLFTCIYYGTEVDF

SEQ ID NO: 415 - protein, 438 aa, Clostridium asparagiforme
MYLPTIARPWYDVIVKEITKVTDLERTGDVTANKFCAEKERLTMRFTLPRDLYHGKDALEALKTLTGKKAIVVVGGGSMKRFGFLDKVVDYLKEAGMEVQLFEGVEPDPSVDTVMKGAEAMRQFGPDWIVAIGGGSPIDAAKAMWAFYEYPETTFEDLITPFSFPTLRTKAKFCAIPSTSGTATEVTAFSVITDYNKGIKYPLADFNITPDIAIVDPALAETMPKKLTAYTGMDAMTHAIEAYVSTLHCEYTDPLALHAIEMINEYLIPSYNGDMEARDKMHDAQCLAGMAFSNALLGIVHSMAHKTGAAYSGGHIVHGCANAMYLPKVIKFNAKEPEAAARYAKIARFIGLTGATDEELTTALIGRLLEMNKALEIPTCIKDYEGGIIDEKEFMDKLPKVAELAVGDACTGSNPRTINPDQMAKLLKCCFYDEEVDF

SEQ ID NO: 416 - protein, 388 aa, Clostridium beijerinckii
MARFTLPRDIYHGEGALEALKTLKGKKAFLVVGGGSMKRFGFLKQVEDYLKEAGMEVELFEGVEPDPSVETVMKGAEAMRNFEPDWIVAMGGGSPIDAAKAMWIFYEYPDFTFEQAVVPFGLPDLRQKAKFVAIPSTSGTATEVTAFSVITNYSEKIKYPLADFNITPDIAIVDPALAQTMPKTLTAHTGMDALTHAIEAYTASLQSNFSDPLAIKAVEMVQENLIKSFEGDKEARNLMHEAQCLAGMAFSNALLGIVHSMAHKVGAVFHIPHGCANAIFLPYVIEYNRTKCENRYGDIARALKLKGNNDAELTDSLIELINGLNDKLEIPHSMKAYGVTEEDFKANLSFIAHNAVLDACTGSNPREIDDATMEKLFECTYYGTKVNL

SEQ ID NO: 417 - protein, 393 aa, Clostridium sp.
MSRFTLPRDIYHGKGCLEELKNLKGKKAILVVGGGSMKRQGFLDRAVDYLKEAGMEVQLFEGVEPDPSVETVMKGAEAMRAFEPDWIVAMGGGSPIDAAKAMWAFYEYPEVTFEDLCIPFNFPELRQKAKFAAIPSTSGTATEVTAFSVITNYQTGVKYPLADFNITPDVAIVDSELVEGLPAKQVAYTGMDALTHAIEAYVSTLNCAFTDPLAIQAIEMVFDYLPASYRGNMDARAEMHTAQCLAGMAFSNALLGIVHSMAHKTGAAFSTGHITHGCANAMYLPFVIKYNAKDPVAAKRYAEIARRVGLQGASEKALINSLCEKIDDFNVQLGIPKTLKEFGINEDEFKEKIARISELAVGDACTGSNPRSITPAEMERLFNCIYYGTEVDF

SEQ ID NO: 418 - protein, 405 aa, Butyrivibrio proteoclasticus; identical to SEQ ID NO: 212

SEQ ID NO: 419 - protein, 396 aa, Clostridium hathewayi
MARFTLPRDLYHGKGALEELKNLKGNKAIVVVGGGSMKRFGFLDRVIDYLKEAGLEVKLFEGVEPDPSVDTVMKGAEMMREFGPDWIVAIGGGSPIDAAKAMWAFYEYPDTTFEDLCIPFNFPTLRTKARFCAIPSTSGTATEVTAFSVITDYNKGIKYPLADFNITPDVAIVDPDLAETMPAKLTAFTGMDAMTHAIEAYVSTLHCDYTDPLALHAIKMIHNDLIDSYNGDMAARDRMHNAQCLAGMAFSNALLGIVHSMAHKTGAAYSGGHIVHGCANAMYLPKVIRFNAKEPEAAKRYAEIARFIDLKGSTDEELVAALIAELRGMNEKLNIPTCIKEYEGGIIDETEFNEKLPSVAELAVGDACTGSNPRPITPAEMEKLLTCCYYDKEVDF

SEQ ID NO: 420 - protein, 393 aa, Dorea formicigenerans
MARFTLPRDIYHGEGALEELKNLKGKKAIVVVGGGAMKRQGFLAKVEDYLKEAGMKVALFEGVEPDPSVETVMKGAKAMQEFEPDWIVAMGGGSPIDAAKAMWAFYEYPEITFEDLITPFSFPELRQKAKFAAIPSTSGTATEVTAFSVITDYAKGIKYPLADFNITPDVAIVDPALVAELPVNQVAYTGMDALTHAIEAYVSTLNCPYTDPLALKAIEMVFDYLPASYRCNMEARAKMHDAQCLAGMAFSNALLGIVHSMAHKTGAAFSTGHITHGLANAMYLPYVIKYNAKDPVAAKRYAEIARFVGLGGASEKALINSLCEKINEFNVILNIPATLKDFGIKEDEFKEKIATISENAVGDACTGSNPRTIDPATMERLFTCIYYGTEVDF

SEQ ID NO: 421 - protein, 388 aa, Clostridium sp.
MARFTLPRDLYHGEGSLEALKTLKGKKAFVVVGGGSMKRFGFLDKVETYLKEAGMEVELFEGVEPDPSVETVMSGAEAMRKFEPDWIVAMGGGSPIDAAKAMWIFYEYPDFTFEQAVIPFGLPELRQKAKFVAIPSTSGTATEVTAFSVITDYKAKIKYPLADFNITPDIAIVDPDLAQTMPAKLVAHTGMDALTHAIEAYTASLRSNFSDPLAMKAIEMVRENLVASYNGDKQARNLMHEAQCLAGMAFSNALLGIVHSMAHKTGAVFHIPHGCANAIFLPYVIQYNRTKCEERYADIARALKLKGETDAELTDSLIEMINDLNTELNIPHTVKEYGVTEEDFKANLEFISHNAVLDACTGSNPREIDDATMEKLFECTYYGTKVNF

SEQ ID NO: 422 - protein, 404 aa, Synergistes sp.
MARFTLPRDLYHGKGSLEALKTLKGTKAIVVVGGGSMKRFGFLDRVEQYLKEAGMEVRLFEGVEPDPSVETVMRGAAVMREFQPDWIVSIGGGSPIDAAKAMWAFYEYPDTTFEALCIPFNFPTLRQKAKFCAIPSTSGTATEVTAFSVITDYAKGIKYPLADFNITPDVAIVDPELAETMPKKLTAHTGMDAMTHAIEAYVSTLNCDYTDPLALHAIEMVSENLVKSYNGDMAARDRMHDAQCLAGMAFSNALLGIVHSMAHKTGAAYSGGHIVHGCANAMYLPKVIRFNAAEPAAAERYAKIAKYLHLEGKTTAELVEALVDHINGLNAQLDIPTRIKDYEGGIIDEKEFMEKLPKVAELAVGDACTGSNPRAITPKQMEELLKSCYFDKEAVRAAKSTQAA

SEQ ID NO: 423 - protein, 388 aa, Clostridium butyricum
MARFTLPRDLYHGEGALEALKTLKGKKAFVVVGGGSMKRFGFLQKVEDYLKEAGMEVELFEGVEPDPSVETVMKGAEAMRNFEPDWIVAMGGGSPIDAAKAMWIFYEYPDFTFEQAVVPFGLPDLRQKAKFVAIPSTSGTATEVTAFSVITNYTEKIKYPLADFNLTPDVAIVDPALAQTMPAKLVAHTGMDALTHAIEAYTASLRSNFSDPLAIKAIEMVNENLVKSYKGDKVARDLMHEAQCLAGMSFSNALLGIVHSMAHKVGAVFHIPHGCANAIFLPYVIQYNRKSCEDRYADIARALKLEGNTDAELTDSLIKLINEFNDKLSIPHSMKEYGIEEAEFKANLEFIAHNAVLDACTGSNPREIDDATMAKLFECTYYGTEVNL

SEQ ID NO: 424 - protein, 393 aa, Clostridium beijerinckii
MARFTLPRDIYHGKGCLEELKNLKGKKAILVVGGGSMKRQGFLDKAVNYLKEAGMEVQLFEGVEPDPSVETVMKGAEAMRAFGPDWIVAMGGGSPIDAAKAMWAFYEYPDVTFEDLCIPFNFPELRQKAKFAAIPSTSGTATEVTAFSVITDYAKGIKYPLADFNITPDVAIVDSELVEALPARQVAYTGMDALTHAIEAYVSTLNCAYTDPLAIQAIEMVFDYLPASFKGNMEARAQMHNAQCLAGMAFSNALLGIVHSMAHKTGAAFSTGHITHGLANAMYLPYVIKYNAKDPVAAKRYAEIARRVGLAGASERALINSLCEKINEFNVILGIPATLKEFGIKEDEFKEKISEIAKNAVGDACTGSNPRAIDPETMERLFTCIYYGTEVDF

SEQ ID NO: 425 - protein, 388 aa, Clostridium butyricum; identical to SEQ ID NO: 217

SEQ ID NO: 426 - protein, 388 aa, Clostridium saccharobutylicum
MMRFTLPRDIYYGKGSLEQLKNLKGKKAMLVLGGGSMKRFGFVDKALGYLKEAGIEVKLIEGVEPDPSVETVFKGAELMRQFEPDWIIAMGGGSPIDAAKAMWIFYEHPEKTFDDIKDPFTVPELRNKAKFLAIPSTSGTATEVTAFSVITDYKTEIKYPLADFNITPDVAVVDSELAETMPPKLTAHTGMDALTHAIEAYVATLHSPFSDPLAMQAVEMINEYLLKSYEGDKEAREQMHYAQCLAGMAFSNALLGICHSMAHKTGAVFHIPHGCANAIYLPHVIKFNSKTSLERYAKIAKQISLPGNTNEELVDSLINLVKELNKKMQIPATLKEYGIDEQEFKDKVDLISERAIGDACTGSNPRQLNKDEMKKIFECVYYGTEVDF

SEQ ID NO: 427 - protein, 388 aa, Clostridium perfringens
MARFTLPRDIYHGKDSLEVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIVPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVNMHLVNSYKGDMEARGEMHEAQCLAGMAFSNALLGIVHSMAHKVGAVFHIPHGCANAIFLPYVIKYNRKACENRYANIARHIGLKGESERELTDDLIDLINKFNKELNIPSSMKEYGIDENEFKANLKFIAHNAVLDPCTGSNPREINDETMEKLYTCAYYGSDVDF

SEQ ID NO: 428 - protein, 388 aa, Clostridium botulinum
MARFTLPRDIYFGSGCLDVLKTIKGKKAVIVIGGGSIKRSGFLDKIEGYLKEANIETKLIEGVEPDPSVETVMNGAAIMREFQPDLIIGVGGGSPIDAAKAMWIFYEYPDFTFEKAVIPFGIPELRQKAKFIAIPSTSGTASEVTAFSVITDYKAKIKYPLADFNLTPDIAIVDSDIAQTMPPKLTAHTGMDALTHAIEAYVAGARSAFSDPLAIQAIVMVRDNLVNSFKGDKEARDQMHIAQCLAGMSFSNALLGITHSMAHKIGAVFHIPHGCANAIFLPYVIEYNRKACEDRYATIARRLNLEGNNDSELVDSLINLINELNVKLNIPSTIKEWGVSEEEFKENVEFMAHNAVLDACTGANPRNIDDKTMMKLYSCAFYGEKVNF

SEQ ID NO: 429 - protein, 396 aa, Clostridium beijerinckii
MARFTLPRDLYHGKGSINELKNLTGKKAIVVVGGGSMKRFGFLDQVVDNLKEAGMEVKLFENVEPDPSVETVMKGAEVMREFEPDWIVAIGGGSPIDAAKAMWVFYEYPETTFEAIIQPFSFPTLRQKAKFCAIPSTSGTATEVTAFSVITDYAKGIKYPLADFNITPDIAIVDPNLAETMPAQLTAHTGMDALTHAIEAYVSTCHSCFTDPLALKAIEMIFDDLQASYEGDMEAREQMHYAQCLAGMAFSNALLGIVHSMAHKTGAAYKNGHIVHGCANAMYLPQVIRFNAKDEVAAKRYADIAKFIDLEGDTTEGLVKALCDKIEAYNVALNIPKCIKDYENGMVPEDEFLEKLPEVAALAISDACTGSNPRIPDQKQMEELLKCCYYGTEVDF

SEQ ID NO: 430 - protein, 388 aa, Clostridium botulinum
MARFTLPRDIYFGAGCLDVLKTIKGKRAVIVIGGGSIKRSGFLDKIEGYLKEANIETRLIEGVEPDPSVETVMNGAKVMREFNPDLIIGIGGGSPIDAAKAMWIFYEYPEFTFEKAVVPFGLPDLRQKAKFIAIPSTSGTASEVTAFSVITDYKAKIKYPLADFNLTPDIAIVDSDIAQTMPPKLVAHTGMDALTHAIEAYVAGLRSAISDPLAIQAIVMVRDNLVKSFEGDKKARDEMHIAQCLAGMAFSNALLGITHSMAHKIGAVFHIPHGCANAIFLPYVIDYNRKVCEDRYATIARSLGLKGNTDKELVDSLIGLINELNVKLNIPSTIKEWGVSEEDFKSNVEFMAHNAVLDACTGANPREINDETMVKLYNCTFYGEKVNF

SEQ ID NO: 431 - protein, 388 aa, Clostridium perfringens
MARFTLPRDIYHGKDSLEVLKSLEGKKAFIVIGGGSMKRFGFLDKVLSYLKEANMETKVFEGVEPDPSVETVMKGAKEMEEFNPDWIVSIGGGSPIDAAKAMWIFYEYPDFTFEKAIIPFGLPKLRRKAKFVAIPSTSGTATEVTAFSVITDYKAKIKYPLADFEITPDIAIVDPSLAETMPEKLVAHTGMDALTHAIEAYTASLRSNFTDPLALKAIEMVKMHLVNSYKGDMEARGEMHEAQCLAGMAFSNALLGIVHSMAHKVGAVFHIPHGCANAIFLPYVIKYNRKACENRYANIARHIGLKGESERELTDALIDLINKFNKELNIPSSMKAYGIEEDDFKANLKFIAHNAVLDPCTGSNPREIDDETMEKLYTCAYYGSDVDF

SEQ ID NO: 432 - protein, 428 aa, Clostridium sp.
MMYNVFVAKQSIEIGYSNVGYIRKGSISVIQVKEMLVMARFTLPRDLYFGENALETLKTIKGNKAVLCLGGGSMKRFGFVDKVVEYLKEAGIETKLIEGIEPDPSVETVMKGAKVMQEFEPDLIIAMGGGSPIDAAKAMWVFYEHPETTFDDIKDPFTIPPLRNKATFIAIPSTSGTATEVTAFSVITDYSTGIKYPLADFEITPDVAIVDPSLAETMPQTLTAHTGMDALTHAIEAYVAGLHSPFSDPLAIHAIEMIFDNLKASYEGDMQARGNMHIAQCLAGMAFSNALLGIVHSLAHKTGAVFNTGHIPHGCANAIYLPYVIQYNAKVCEDRFAMIARKLGLPGVTDKALTLSLIKAVEDLRTSLHIVSNLKDFGVNEDEFLEKKHMIAERAVGDACTGSNPRETSVEDMEKILECIYYGKDVTF

SEQ ID NO: 433 - protein, 388 aa, Clostridium sp.
MMRFTLPRDVYYGKGSLEQLKNLKGKKAVLVLGGGSMKKFGFVDKAVSYLNEAGIEVKLIEGVEPDPSVETVFKGAEVMREFNPDWIISMGGGSPIDAAKAMWIFYEYPDFTFEQAVVPFGLPELRQKAKFLAIPSTSGTATEVTAFSVITDYKTEIKYPLADFNITPDIAIVDSELAETMPPTLTAHTGMDALTHAIEAYVATLHSPFTDPLAIQAIEMINEYLAKSYEGDNEAREQMHYAQCLAGMAFSNALLGITHSMAHKTGAVFHIPHGCANAIYLPYVIDFNKKAASDRYAKIAKSLGLDGQTNDELIDSLTSLIQEFNKKMSIPLTLKEYGIDEEEFKAKVDLIAERAVGDACTGSNPRQINKDEMKKIFECVYYGNKVDF

SEQ ID NO: 434 - protein, 393 aa, Clostridium beijerinckii
MGRFTLPRDIYHGKGCLEELKNLKGKKAILVVGGGSMKRQGFLDKAVNYLKEAGMEVELFEGVEPDPSVETVMRGAEAMRKFEPDWIISMGGGSPIDAAKAMWAFYEYPDVTFEELCIPFNFPELRQKAKFCAIPSTSGTATEVTAFSVITNYETGVKYPLADFNITPDVAIVDSELVEGLPAKQVAYTGMDALTHAIEAYVSTLNCAYTDPLAIQAIEMVFDYLPASFRGNMDARAHMHDAQCLAGMAFSNALLGIVHSMAHKTGAAFSTGHITHGCANAMYLPYVIKYNAKDPVAAKRYAEIARKVGLPGASEKALINSLCEKIDDFNVQLNIPKTLKEFGINEDEFKEKIAKISELAVGDACTGSNPRAIEPATMEKLFTCIYYGTEVDF

SEQ ID NO: 435 - protein, 388 aa, Clostridium botulinum
MARFTLPRDIYFGAGCLDVLKTIKGKKAVIVIGGGSIKRSGFLDKIERYLKEANIETRLIEGVEPDPSVETVMNGASVMREFEPDLIIGVGGGSPIDAAKAMWIFYEYPDFTFEKAVIPFGIPELRQKAKFIAIPSTSGTASEVTAFSVITDYKAKIKYPLADFNLTPDIAIVDSDIAQTMPPKLTAHTGMDALTHAIEAYVAGARSAFSDPLAIQAIVMVRDNLVNSFKGDREARDQMHIAQCLAGMSFSNALLGITHSMAHKIGAVFHIPHGCANAIFLPYVIEYNRKACEDRYATIARRLELKGNTDSELVDSLINLINELNVKLNIPSTIKEWGVSEEDFKKNVEFMAHNAVLDACTGANPREIDDETMTKLYNCTFYGEKVDF

SEQ ID NO: 436 - protein, 395 aa, Clostridium clostridioforme
MRFTLPRDLYHGKDALEALKTLAGKKAIVVVGGGSMKRFGFLDKVIDYLKEAGMEVQLFEGVEPDPSVDTVLKGAAAMQEFEPDWIVAVGGGSPIDAAKAMWAFYEYPDTSFEDLITPFSFPTLRTKAKFCAISSTSGTATEVTAFSVITDYKKGIKYPLADFNITPDVAIVDPALAETMPKKLTAHTGMDAMTHAIEAYVSTLHCEYTDPLALHAIEMINEYLIPSYNGDMEARDKMHDAQCLAGMAFSNALLGIVHSMAHKTGAAYSGGHIVHGCANAMYLPKVIKFNSKEPEAAARYAKIAKFINLPGTTDEELTDALIARLLEMNKALDIPACIKDYEGGIIDEKEFMDKLPKVAELAVGDACTGSNPRTITPEVMEQLLKCCYYDEEVDF

SEQ ID NO: 437 - protein, 393 aa, Dorea longicatena
MARFTLPRDVYHGKGCLSELKNLKGKKAVLVVGGGSMKRQGFLDKAVNYLKEGGMEVKLIEGVEPDPSVETVMKGAKVMQEFEPDWIVAMGGGSPIDAAKAMWVFYEYPDETFENIITPFSFPELRQKAKFAAIPSTSGTATEVTAFSVITDYNTGIKYPLADFNITPDVAIVDPELVEGLPQKQVAYTGMDALTHAIEAYVSTLNCPFTDPLALQAIEMVLDYLPASYNLNMEAREQMHYAQCLAGMAFSNALLGIVHSMAHKTGAAFSTGHIPHGCANAIYLPYVIKYNAKDKVAASRYAEIARRMGLPGTSEKALINSLCAKIDEFNAKMNIPKTLKEFGINEEEFKEKLDKIAELAVGDACTGSNPRAIDPATMAKLFTCTYYGTEVDF

SEQ ID NO: 438 - protein, 391 aa, Anaerostipes sp.
MGRFTLPRDLYFGKGSLETLKTLDGKKAVLVLGGGSMKRFGFVDQVVDYLKEAGIETKLIEGVEPDPSVETVFKGAEVMRDFEPDWIIAMGGGSPIDAAKAMWIFYEHPETTFDDVKDPFTVPPLRNKAKFLAIPSTSGTATEVTAFAVITDYSSGIKYPLADFEITPDIAIVDPALAETMPPKLTSYTGMDALTHAIEAYVAGLHSPFSDPLALEAIEMVFDNLKASYEGDMTARENMHYAQCLAGMSFSNALLGIVHSLAHKTGAVFDTGHITHGCANAIYLPYVIQFNAKVCEERYAAIARRIGLPGNSDKALTLSLIEAIREMNKKLNIVSNLKDFGINEEEFNQKKRMIAERAVADACTGSNPRETSVDDMEKVLECIYYGKDVTF

SEQ ID NO: 439 - protein, 468 aa, Clostridium saccharoperbutylacetonicum; identical to SEQ ID NO: 237

SEQ ID NO: 440 - protein, 468 aa, Clostridium saccharoperbutylacetonicum
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 441 - protein, 468 aa, Clostridium beijerinckii; identical to SEQ ID NO: 70

SEQ ID NO: 442 - protein, 468 aa, Clostridium beijerinckii; identical to SEQ ID NO: 239

SEQ ID NO: 443 - protein, 468 aa, Clostridium sp.
MNKDTTISETENLKFKTNIKNADLKNYENSTSYSGVFEDVEVAINKAITAQKEFSLYYTKEQREKILTEIRKATLKNKKILAKMILDETHMGRYEDKILKHELVAKYTPGIEDLTTTAWSGDNGLTVVEMAPYGVIGAITPSTNPTETVICNSIGMIAAGNAVVFNGHPSAKKCVAFAVDMINKAIVSCGGPKNLITAVKNPTMESLDAIIKHPEIKLLCGTGGPGMVKTLLNSGKKAIGAGAGNPPVIVDDTADIEKAGKNIIEGCSFDNNLPCIAEKEVFVFDNVADNLIDNMLKNNAVIINKDKITKLLNLILQKNNETQEYNINKKWVGKDAKLFLNEIDVEAPSSVRCIICEVEPDHPFVMTELMMPILPIVRVKNIDDAIQYAKIAEQSRKHSAYIYSKNIDNLNRFEKEIDTTIFVKNAKSFAGVGYNAEGFTTFTIAGCTGEGITSARNFTRQRRCVLAG

SEQ ID NO: 444 - protein, 469 aa, Clostridium saccharobutylicum
MNNNLFVSPETKDLKLRTNVENLKFKGCEGGSTYIGVFENAETAIDEAVNAQKRLSLYYTKEQREKIITEIRKVTLKNKEILAQMILEETHMGRYEDKILKHELVAKYTPGTEDLATTAWSGDNGLTVVEMSPYGVIGAITPSTNPTETIICNSIGMIASGNAVVFNGHPGAKKCVAFAVDMINRAIISCGGPRNLVTAIKNPTMESLDAIIKHPAIKLLCGTGGPGMVKTLLSSGKKSIGAGAGNPPVIVDDTADIEKAGKSIIEGCSFDNNLPCIAEKEVFVFENVADDLIKNMLKNNAVIINKDQVSRLVNLVLQKNNETSEYTINKKWVGKDAKLFLDEIDVESSSDVRCIICEVDADHPFVMTELMMPILPIVRVKDIDEAIKYAKIAEQNRKHSAYIYSKNIENLNRFEKEIDTTIFVKNAKSFAGVGYGAEGFTTFTIAGCTGEGITSARNFTRQRRCVFVG

SEQ ID NO: 445 - protein, 467 aa, Clostridium botulinum; identical to SEQ ID NO: 235

SEQ ID NO: 446 - protein, 527 aa, Caldalkalibacillus thermarum; identical to SEQ ID NO: 240

SEQ ID NO: 447 - protein, 480 aa, Pelosinus fermentans
MSIDQALIEKITLEILTKMQTGAKAAPAGYGDGIFETVDEAVAAARKAYQELKTLSLEKREVLIKAMRDVAYENATILAQMAVDESGMGRVSDKIIKNQVAALKTPGTEDLTTQAWSGDNGLTLIEMGPYGVIGAITPTTNPTETVICNGIGMIAAGNTVFFSPHPTAKNTSMKIITLLNQAIVKAGGPNNLLTSVANPSIKAANEMMKHPGINMLVATGGPGVVKAVLSSGKKAIGAGAGNPPVIVDETADIEKAARDIVAGCSFDNNLPCIAEKEVIAIGSIADRLITYMQKYGAYLISGSNIDRLLDVIMTVQEEKIAEGCTDKPKRSYGINKDYVGKDAKYLLSKIGIDVPDSVRVVLCETPADHPFVIEELMMPVLPVVQVKDIDEAIEVAVRVEHGNRHTAAMHSKNVDHLTRFARAVETTIFVKNAPSYAGIGVGGEGFTSFTLAGPTGEGITSPRSFTRQRRCVLVDAFSIV

SEQ ID NO: 448 - protein, 479 aa, Thermoanaerobacterium thermosaccharolyticum
MEINDNMISEIIERVLKEVQKKSINDRYQNGIYDRMEDAIEAAYEAQKKLMKMSIEQRERLISAMRKAILDNAKSCAKLSVEETGMGRVDHKYLKLKLVAEKTPGTEVLTTKAYSGDKGLTLVEMAPFGVIGSITPSTNPAETVCCNSIGMIAAGNTVVFSPHPGAIKSSLMAVEFLNKAIIEAGGPENLITSVRKPSIEFTDVMINHPKINLLVATGGPAIVKKVLSSGKKAIGAGAGNPPCVVDETADIKKAARDIILGCTFDNNLPCIAEKEVIAVESIYEELIENMKKNGAYEITDDEAEKLADIVLTKKEELKAEGCSINRPKFEYSVNKKWVGKDAKVLLEQIGINVGDDIVCIIYRCDKQHPFVQEELMMPILPIVKVKNIDEAINVAVEVEHGNHHTAEMHSKNIDNLTRFAKAINTTIFVKNAPSYAGIGFGGEGYTTFTIAGPTGEGLTCAATFTRQRRCVMVDSFRIV

SEQ ID NO: 449 - protein, 480 aa, Pelosinus fermentans; identical to SEQ ID NO: 238

SEQ ID NO: 450 - protein, 479 aa, Desulfosporosinus sp.
MSVDQALIRKITSEILAKMQNRTVSACQDCNGIFTTVDEAVAAARIAYQELRTLSLEKREELIKAMRNVALENATMLAEMAVKESGMGRVEDKIIKHKLVAVKTPGTEDLRTEAWSGDSGLTLVEMGPYGVIGAITPTTNPVATIICNGIGMIAAGNAVFFSPHPTAKNTSIKTITLLNEAIVKAGGPMNLLTSVADPSISAANAMMKHAGINLLVATGGPGVVKAVLSSGKKAIGAGAGNPPVIVDETADIEKAARDIIAGCSFDNNLPCIAEKEVIAVGCIADRLISNMQKYGAYLISGSKIDQMLDVVMTATEEKMAEGCTAKPIKRYGINKDFVGKDAKYILTQIGLDVPDTIKVILCETPADHPFVIEELMMPILPVVQVKDIDAAIELAVKVEHGNRHTAMMHSKNVDNMTRFAKAIETTIFVKNAPSYAGIGVGGEGFCTFTIAGPTGEGLTTARSFTRQRRCVLVDSFSII

SEQ ID NO: 451 - protein, 482 aa, Clostridium methylpentosum
MEITPNQIDQIVANVMAQLGGSAAPAASYDSTQYSGRKYIGIYATMTEAIDAVADAYKVLRSMTVDQREKIIEKIREFTRAEAEIMAKMGVEETGMGKVEHKTLKHHLVADKTPGTEDIQTEAMSGDGGLTLLEMAPFGIIGAISPSTNPSETVLCNSMGMIAGANAVVFNPHPSAICTSNYAVDLVNRASLAAGGPANLCCSVVKPTMQSADDMVKDPRVKMLVCTGGPGVVRAMLSSGKKAIGAGAGNPPVIVDDTADIRKAAKDIIDGCTFDNNLPCIAEKEVFAFSNIADELMYYMQQNGAYFISGEMADRLAKIVLVEKKNEKTGKISYSVSRDWVGRDAKKFLAALDIEVGDDVRCVICETDENHLFVQTELMMPILPIVRVNNIDEAVRMAVRAEHGNRHTAHMHSKNIDNLTKFARAVETTIFVKNAPSYAGIGFGSEGHTTFTIAGPTGEGLTSARSFTRKRRCVMSDSFNIV

SEQ ID NO: 452 - protein, 467 aa, Thermoanaerobacterium saccharolyticum
MKVKEEDIEAIVKKVLSEFNFEKNTKSFRDFGVFQDMNDAIRAAKDAQKKLRNMSMESREKIIQNIRKKIMENKKILAEMGVSETGMGKVEHKIIKHELVALKTPGTEDIVTTAWSGDKGLTLVEMGPFGVIGTITPSTNPSETVLCNSIGMIAAGNSVVFNPHPGAVNVSNYAVKLVNEAVMEAGGPENLVASVEKPTLETGNIMFKSPDVSLLVATGGPGVVTSVLSSGKRAIGAGAGNPPVVVDETADIKKAAKDIVDGATFDNNLPCIAEKEVVSVDKITDELIYYMQQNGCYKIEGREIEKLIELVLDHKGGKITLNRKWVGKDAHLILKAIGIDADESVRCIIFEAEKDNPLVVEELMMPILGIVRAKNVDEAIMIATELEHGNRHSAHMHSKNVDNLTKFGKIIDTAIFVKNAPSYAALGYGGEGYCTFTIASRTGEGLTSARTFTKSRRCVLADGLSIR

SEQ ID NO: 453 - protein, 467 aa, Thermoanaerobacterium xylanolyticum
MKVKEEDIEAIVKKVLSEFNLEKTTSKYGDVGIFQDMNDAISAAKDAQKKLRNMPMESREKIIQNIRKKIMENKKILAEMGVRETGMGRVEHKIVKHELVALKTPGTEDITTTAWSGDKGLTLVEMGPFGVIGAITPSTNPSETVLCNSIGMIAAGNSVVFNPHPGAVNVSNYAVKLVNEAAMEAGGPENLVVSVEKPTLETGNVMFKSSDVSLLVATGGPGVVTAVLSSGKRAIGAGAGNPPVVVDETADIKKAAKDIIDGATFDNNLPCIAEKEVVSVDKITDELIYYMQKNGCYKIEGREIEKLIELVLDHEGGKTTLNRKWVGKDAHLILKAIGIDADESVRCIIFEAEKDNPLVVEELMMPILGIVRAKNVDEAIMIATELEHGNRHSAHMHSKNIDNLTKFGKIIDTAIFVKNAPSYAALGYGGEGYCTFTIASRTGEGLTSARTFTKSRRCVLADGLSIR

SEQ ID NO: 454 - protein, 477 aa, Acetonema longum
MVDQTLIEQITRAVLTQMKAGKDAAVSGDGIFATVDQAVAAARQAYQELRLLTLEKRETLIRAIRDAAFANAAVIAQMAVQESGMGRVEDKILKNQLAARKTPGTEDLTSRAWSGDHGLTLVEMAPYGVIGAITPTTNPSETVICNSIGMIAAGNSIVFSPHPTAQNTSLTTIRLLNEAIVKAGGPDNLLTAVAEPSIEAANAMMRHPGIQMLVATGGPAVVKAVLSSGKKAIGAGAGNPPAVVDETADIAKAAKDIVAGCSFDNNLPCIAEKEIIAVGRIADELISYLQKYGAYLISGRDIERLMEVVLTERTEEMAPGCVGKPRRVYGVNKDYIGKDAKFILSKINIQAPDHIRVILCETPADHPFVLEELMMPVLPLVSVRDIDAAIDLAVKVEHGNRHTAVMHSKNVDYMTRLAKAIETTIFVKNAPSYAGIGVGGEGFTTFTIAGPTGEGLTSARSFTRQRRCALVDAFSIV

SEQ ID NO: 455 - protein, 465 aa, Clostridium beijerinckii
MSVDAQKIEKLVRKILEEMEEKKKPAETECEWGIFDHMNQAIEAAEIAQKELVQLSLGQRGKLIEAIRKAAKENAEKFARMAVDETGMGKYEDKIVKNLLAAEKTPGIEDLRTEVFSGDDGLTLVELSPYGVIGAITPTTNPTETIICNSIGMIAAGNAVVFSPHPRAKNTSLYAIKIFNQAIVEAGGPKNLITTVANPSIEQAEIMMKHKTIKMLVATGGPGVVKAVLSSGKKAIGAGAGNPPVVVDETADIEKAAKDIIAGCSFDNNLPCVAEKEVIAVESIADRLIDYMKKHGAYEITNKEQIQQLTDLVVENGHANKEFVGKDAAYILKHIGINVPPDIRVAIMEVDGKHPLVTVELMMPILPIVRVKNVDQAIELAVEVEHGFRHTAIMHSKNVDHLTKFAKAIQTTIFVKNAPSYAGIGVGGEGYATFTIAGPTGEGLTSAKDFARKRKCVLVDALSIR

SEQ ID NO: 456 - protein, 463 aa, Geobacillus sp.
MDAQKIEKLVRKILEEMEEKKKPAETECEWGIFDHMNQAIEAAEIAQKELVQLSLGQRGKLIEAIRKAAKENAEKFARMAVDETGMGKYEDKIVKNLLAAEKTPGIEDLRTEVFSGDDGLTLVELSPYGVIGAITPTTNPTETIICNSIGMIAAGNAVVFSPHPRAKNTSLYAIKIFNQAIVEAGGPKNLITTVANPSIEQAEIMMKHKTIKMLVATGGPGVVKAVLSSGKKAIGAGAGNPPVVVDETADIEKAAKDIIAGCSFDNNLPCVAEKEVIAVESIADRLIDYMKKHGAYEITNKEQIQQLTDLVVENGHANKEFVGKDAAYILKHIGINVPPDTRVAIMEVDGKHPLVTVELMMPILPIVRVKNVDQAIELAVEVEHGFRHTAIMHSKNVDHLTKFAKAIQTTIFVKNAPSYAGIGVGGEGYATFTIAGPTGEGLTSAKDFARKRKCVLVDALSIR

SEQ ID NO: 457 - protein, 463 aa, Geobacillus thermoglucosidasius
MDAQKIEKLVRKILEEMEEKKKPAETECEWGIFDHMNQAIEAAEIAQKEFVQLSLGQRGKLIEAIRKAAKENAEKFARMAVDETGMGKYEDKIVKNLLAAEKTPGIEDLRTEVFSGDDGLTLVELSPYGVIGAITPTTNPTETIICNSIGMIAAGNAVVFSPHPRAKNTSLYAIKIFNQAIVEAGGPKNLITTVANPSIEQAEIMMKHKTIKMLVATGGPGVVKAVLSSGKKAIGAGAGNPPVVVDETADIEKAAKDIIAGCSFDNNLPCVAEKEVIAVESIADRLIDYMKKHGAYEITNKEQIQQLTDLVVENGHANKEFVGKDAAYILKHIGINVPPDIRVAIMEVDGKHPLVTVELMMPILPIVRVKNVDQAIELAVEVEHGFRHTAIMHSKNVDHLTKFAKAIQTTIFVKNAPSYAGIGVGGEGYATFTIAGPTGEGLTSAKDFARKRKCVLVDALSIR

SEQ ID NO: 458 - protein, 463 aa, Bacillus azotoformans
MAVEAKAIEEIVKKILEEMMIKKDACITGYGIFEDMNEAIEAATIAQKELLKLSLEQRGNLITAIRKAAKDNAETFAQMAVDETGMGNYGDKVIKNLIAAEKTPGIEDLTTEAFSGDHGLTLVELSPYGVIGSITPTTNPTETVICNSIGMIAAGNAVVFSPHPTAKNTSLKAIEVINKAIIKAGGPPNLITSVANPTIDQANIMMKHKKIKLLVATGGPGVVKAVLSSGKKAIGAGAGNPPAVVDETANLEKAARDIIDGCSFDNNLPCTAEKEVIVVDSVADYLVSYMKKHGAFLITDKEQIQKLTELVVDNGHANKELVGKSVAHILQRIGIEVPSDARVAILNVERNHPLVKAELMMPILPVVRVENVDAAIELAVEAEQGFRHTAIMHSTNIDNLTKFSKEIQTTIFVKNGPSYAGIGIGGEGYATFTIAGPTGEGLTSAKDFARRRKCVLVDGLSIR

SEQ ID NO: 459 - protein, 503 aa, Clostridium sticklandii
MKAGDIVQDFITERDVEKIIEQVLSKLEPVIEQVKPKEINMLPNKTNIDFSQNANGIFESIDLAVESALEAHIILTSYKLEDREKMIQSIRKEVLGDIENIARLVYEETKLGKYEDKIAKINLAASKTPGTEDIKTSAISGDYGLTIEEMAPFGVIGAVTPVTNPVETLINNAISMISGGNSVVFNVHPSSKKSSAYTVELINKAVLKAGGPQNLVTMVKEPTIETVNQLSSHPRISMMVGTGGPGLVKSLLKSGKKTIGAGAGNPPVVVDETADMNLAAKGIIEGASFDNNILCIAEKEVFVVNEVADDLIYNMLSSGAYMLNQEELEKVMKLTLVEDEDLGAKSCTLSPKKKYHVHKNWVGKDASKILSEIGITKQDVKLLICEVDSDHPYVTLEQMMPILPLVRCSDVDEAIKLAVKAEGTNKHTASIFSRNVDNMTKFARAINTTIFVKNAPTLAGVGYKGEGNATFTIAGPTGEGITSAKTFTRVRRCVLAEGGFRIV

SEQ ID NO: 460 - protein, 482 aa, Clostridium beijerinckii
MAIEAYQIEKIVEEVMRKMVSGGSGDSFAGKAKGIFESVDEAVKAAKAAQKELVAMRIEKREMLLKAMREAAIAHAEELARLAVEETGMGRVTDKIIKNRVAAEKTPGTENLQPSAVTGDRGLTLIERAPYGVIGAITPSTNPCATVINNSISMVAAGNSVVFSVHPGAKKASLLTVEILNEAIEKAGGPANVLTAVASPSLENTNALMKHPDIKLLVATGGPGLVKAVLSSGKKAIGAGAGNPPALVDETADLERAAKSIVAGASFDNNLPCIAEKEVIVVDYVANQLISYMKQNGAYLANDREIKALMDLVLTKNENLKAEGCTVKPEKLYGGINKEYVGKDAAYIMKKIGVDIPEDTKLIICEVDEDHPFVLEELMMPILPIVRVPNVQKAIEVGVRVEHGNRHTAVMHSQNIDNLSAFARAVQTTIFVKNGPSYAGIGIGGEGYTTFTIAGPTGEGLTAASSFTRQRRCVLVDGFSIV

SEQ ID NO: 461 - protein, 462 aa, Clostridium sp.
MSVNEQMIQDIVSEVMAKMQIASEVSDNHGIFADMNEAIEAAKKAQKIVGRMSMDQREKIISNIRKKTVENAEILARMGVQETGMGNVGHKILKHQLVAEKTPGTEDITTTAWSGDRGLTLIEMGPFGVIGAITPCTNPSETVLCNTIGMLAGGNTVVFNPHPGAIKTSIFAINMINEASLEAGGPDNIACTVEKPTLESSNIMMKHKAIHLIAATGGPGVVTAVLSSGKRGIGAGAGNPPALVDETADIRKAAEDIVNGCTFDNNLPCIAEKEIVAVDSVADELMHYMVSEQGCYLASKEEQEALTAVVLKDGRLNRNCVGRDAKTLLGMIGVSVPDNIRCITFEGPKEHPLIATELMMPILGVVRAKDFDDAVEQSVWLEHGNRHSAHIHSKNVDNITKYAKAIDTAILVKNGPSYAAIGFGGEGFCTFTIASRTGEGLTSASTFTKRRRCVMSDSLCIR

SEQ ID NO: 462 - protein, 470 aa, Fusobacterium sp.
MRGELMEFEVNNIEKIVELIMKKMAESNISTAGNSKNGVFDNVDEAIEEAKKAQAILFSSKLELREKIIASIRDTLKSHVTELAELAVKETGMGRVSDKELKNKIAIEKTPGLEDLKAFAFSGDDGLTVMELSPYGVIGAITPSTNPSETVICNSIGMIAAGNAVIFAPHPGAKRTSIRTVELINEAIRKVGGPDNLVVTIREPSIENTEKIIANPNIKMLVATGGPGVVKTVMSSGKKAIGAGAGNPPVLVDETADIEKAAKDIIAGCSFDNNLPCTAEKEVVAVDSIVNYLIFEMQKNGAYLLKDKKLIEKLLSLVLKNNSPDRKYVGRDAKYLLKQIGIEVGDEIKVIIVETDKNHPFAVEELLMPILPIVKVKDALEGIKVAKELERGLRHTAVIHSKNIDILTKYAREMETTILVKNGPSYAGIGIGGEGHVTFTIAGPTGEGLTSAKSFARNRRCVLVGGFSIK

SEQ ID NO: 463 - protein, 470 aa, Fusobacterium sp.
MRGELMEFEVNNIEEIVELIMKKMAESNISTAGNSKNGVFDNVDEAIEEAKKAQAILFSSKLELREKIIASIRDTLKNHVSELAELAVKETGMGRVADKELKNKIAIEKTPGLEDLKAFAFSGDDGLTVMELSPYGVIGAITPSTNPSETVICNSIGMIAAGNAVIFAPHPGAKRTSIRTVELINEAIRKVGGPDNLVVTIREPSIENTEKIIANPNIKMLVATGGPGVVKTVMSSGKKAIGAGAGNPPVLVDETADIEKAAKDIIAGCSFDNNLPCTAEKEVVAVDSIVNYLIFEMQKNGAYLLKDKELIEKLLSLVLKNNSPDRKYVGRDAKYLLKQIGIEVGDEIKVIIVETDKNHPFAVEELLMPILPIVKVKDALEGIKVAKELERGLRHTAVIHSKNIDILTKYAREMETTILVKNGPSYAGIGIGGEGHVTFTIAGPTGEGLTSAKSFARNRRCVLVGGFSIK

SEQ ID NO: 464 - protein, 462 aa, Ruminococcus sp.
MPISENMVQEIVQEVMAKMQIADAPAGKHGVFKDMNDAIEAAKKAQLVVKTMSMDQREKIITCIRKKIKENAEVLARMGVEETGMGNVGDKILKHHLVADKTPGTEDITTTAWSGDRGLTLIEMGPFGVIGAITPCTNPSETVLCNTMGMLAGGNTVVFNPHPAAVKTSIYAINLLNEASLESGGPDNIAVTVEKPTLETSNIMMKHKDIHLIAATGGPGVVTAVLSSGKRGIGAGAGNPPALVDDTADIRKAAQDIVNGCTFDNNLPCIAEKEVVAVSSVVDELMHYMITENDCYLASKEEQDKLVETVLAGGKLNRKCVGRDAKTLLSMIGVQAPANTRCIIFEGPKEHPLITTELMMPILGIVRAKDFDDAVEQAVWLEHGNRHSAHIHSKNIDNITKYAKAIDTAILVKNGPSYSALGFGGEGFCTFTIASRTGEGLTSASTFTKRRRCVMTDSLCIR

SEQ ID NO: 465 - protein, 465 aa, Fusobacterium nucleatum
MEFEVNNIEEIVELIMKKMAESNISTAGNSKNGVFDNVDEAIEEAKKAQAILFSSKLELREKIIASIRDTLKNHVTELAELAVKETGMGRVADKELKNKIAIEKTPGLEDLKAFAFSGDDGLTVMELSPYGVIGAITPSTNPSETVICNSIGMIAAGNAVIFAPHPGAKRTSIRTVELINEAIRKVGGPDNLVVTIREPSIENTEKIIANPNIKMLVATGGPGVVKTVMSSGKKAIGAGAGNPPVLVDETADIEKAAKDIIAGCSFDNNLPCTAEKEVVAVDSIVNYLIFEMQKNGAYLLKDKELIEKLLSLVLKNNSPDRKYVGRDAKYLLKQIGIEVGDEIKVIIVETDKNHPFAVEELLMPILPIVKVKDALEGIKVAKELERGLRHTAVIHSKNIDILTKYAREMETTILVKNGPSYAGIGIGGEGHVTFTIAGPTGEGLTSAKSFARNRRCVLVGGFSIK

SEQ ID NO: 466 - protein, 465 aa, Fusobacterium nucleatum
MEFEVNNIEEIVELIMKKMTEGGVSTSNNSTNGVFKNVDEAIAEAKKAQTVLFSSKLELRERIIASIRDTLKSHITELSELAVKETGMGRVADKELKNRIAIEKTPGLEDLKAFAFSGDDGLTVMELSPYGVIGAITPSTNPSETVICNSIGMIAAGNAVIFAPHPGAKRTSIRAVELINEAIKKAGGPDNLVVTIAEPSIENTEKIIANPNIKMVVATGGPGVVKTVMSSGKKAIGAGAGNPPVLVDETADIEKAAKDIIAGCSFDNNLPCTAEKEVIAVDSIVNYLIFEMQKNGAYLLKDKELIEKLLSIVLKNNSPDRKYVGKDAKYLLKQIGIEVGDEIKVIIVETDKNHPFAVEELLMPILPIVKVKDALEGIKVAKELEKGLRHTAVIHSKNIDILSKYAREMETTILVKNGPSYAGIGIGGEGHVTFTIAGPTGEGLTSARSFARNRRCVLVGGFSIK

SEQ ID NO: 467 - protein, 470 aa, Fusobacterium sp.
MRGELMEFEVNNIEEIVELIMKKMAESNISTAGNSKNGVFDNVDGAIEEAKKAQAILFSSKLELREKIIASIRDTLKNHVTELAELAVKETGMGRVADKELKNKIAIEKTPGLEDLKAFAFSGDDGLTVMELSPYGVIGAITPSTNPSETVICNSIGMIAAGNAVIFAPHPGAKRTSIRTVELINEAIRKVGGPDNLIVTIREPSIENTEKIIANPNIKMLVATGGPGVVKTVMSSGKKAIGAGAGNPPVLVDETADIEKAAKDIIAGCSFDNNLPCTAEKEVVAVDSIVNYLIFEMQKNGAYLLKDKELIEKLLSLVLKNNSPDRKYVGRDAKYLLKQIGIEVGDEIKVIIVETDKNHPFAVEELLMPILPIVKVKDALEGIKVAKELERGLRHTAVIHSKNIDILTKYAREMETTILVKNGPSYAGIGIGGEGHVTFTIAGPTGEGLTSAKSFARNRRCVLVGGFSIK

SEQ ID NO: 468 - protein, 465 aa, Fusobacterium nucleatum
MEFEVNNLEEIVELIMKKMSESSISTSSNSKNGVFENVDEAIAEAKKAQTILFSSKLELRERIIASIRDTLKPYITELSELAVKETGMGRVSDKEIKNRIAIEKTPGLEDLKAFAFSGDDGLTVMELSPYGVIGAITPSTNPSETVICNSIGMIAAGNAVIFAPHPGAKRTSIRAVELINEAIKKVGGPDNLIVTITEPSIENTEKIIANPNIKMVVATGGPGVVKTVMSSGKKAIGAGAGNPPVLVDETADIEKAAKDIIAGCSFDNNLPCTAEKEVIAVDSIVNYLIFEMQKNGAYLLKDKDLIEKLLSIVLKNNSPDRKYVGKDAKYLLKQIGIEVGDEIRVIIVETSKDHPFAVEELLMPILPIVKVKDALEGIKVAKELEKGLRHTAIIHSKNIDILSKYAREMETTILVKNGPSYAGIGIGGEGHVTFTIAGPTGEGLTSARSFARNRRCVLVGGFSIK

SEQ ID NO: 469 - protein, 465 aa, Fusobacterium nucleatum
MEFEVNNIEEIVELIMKKMSESGVSTSNNSTNGVFENVDEAIAEAKKAQTVLFSSKLELRERIIASIRDTLKTHITELSELAVKETGMGRVADKELKNRIAIEKTPGLEDLKAFAFSGDDGLTVMELSPYGVIGAITPSTNPSETVICNSIGMIAAGNAVIFAPHPGAKRTSIRAVELINEAIKKAGGPDNLVVTIAEPSIENTEKIIANPNIKMVVATGGPGVVKTVMSSGKKAIGAGAGNPPALVDETADIEKAAKDIIAGCSFDNNLPCTAEKEVIAVDSIVNYLIFEMQKNGAYLLKDKELIEKLLSIVLKNNSPDRKYVGKDAKYLLKQIGIEVGDEIKVIIVETDKNHPFAVEELLMPILPIVKVKDALEGIKVAKELEKGLRHTAVIHSKNIDILSKYAREMETTILVKNGPSYAGIGIGGEGHVTFTIAGPTGEGLTSARSFARNRRCVLVGGFSIK

SEQ ID NO: 470 - protein, 470 aa, Fusobacterium sp.
MRGELMEFEVNNIEEIVELIMKKMAESNISTAGNSKNGVFDNVDEAIEEAKKAQAILFSSKLELREKIIASIRDTLKNHVTELAELAVKETGMGRVADKELKNKIAIEKTPGLEDLKAFAFSGDDGLTVMELSPYGVIGAITPSTNPSETVICNSIGMIAAGNAVIFAPHPGAKRTSIRTVELINEAIRKVGGPDNLIVTIREPSIENTEKIIANPNIKMLVATGGPGVVKTVMSSGKKAIGAGAGNPPVLVDETADIEKAAKDIIAGCSFDNNLPCTAEKEVVAVDSIVNYLIFEMQKNGAYLLKDKELIEKLLSLVLKNNSPDRKYVGRDAKYLLKQIGIEVGDEIKVIIVETDKNHPFAVEELLMPILPIVKVKDALEGIKVAKELERGLRHTAVIHSKNIDILTKYAREMETTILVKNGPSYAGIGIGGEGHVTFTIAGPTGEGLTSAKSFARNRRCVLVGGFSIK

SEQ ID NO: 471 - protein, 481 aa, Clostridium asparagiforme
MEIETRDIERIVRQVMAAMEQQGTIAGGAYPPAPGITAPRGDNGVFERVEDAIDAAWAAGRVWAFHYKVEDRRRVIEAIRVMARENARTLAQMVRDETGMGRVEDKVEKHLAVADKTPGVECLTTDAISGDGGLMIEEYAPFGVIGAITPSTNPTETVIHNTISMIAGGNSVVFNVHPGAKKCCAFCLQLLNKTIVENGGPANLITMQRDPTMDAVNKMTSSPKIRLMVGTGGMGMVNALLRSGKKTIGAGAGNPPVIVDDTADVKLAARELYWGASFDNNLFCFAEKEVFVMEASADGLIRGLVEQGAYLLTPAETEAIVKLALIQKDGKYEVNKKWVGKDAGLFLQAIGVSGHENTRLLICDVPKCHPYVMVEQLMPVLPIVRCRTFDECIQCSVEAEQGNRHTSSIFSTNVYNMTKFGKEIETTIYVKNGATLRGLGIGGEGHTTMTIAGPTGEGLTCARSFTRRRRCMLAEGGLRII

SEQ ID NO: 472 - protein, 462 aa, Clostridium phytofermentans
MTVNEQLVQDIIKNVVASMQLTQTNKTELGVFDDMNQAIEAAKEAQLVVKKMSMDQREKIISAIRKKTIEHAETLARMAVEETGMGNVGHKILKHQLVAEKTPGTEDITTTAWSGDRGLTLVEMGPFGVIGAITPCTNPSETIICNTIGMLAGGNTVVFNPHPAAIKTSNFAVQLINEASLSAGGPVNIACSVRKPTLDSSKIMMSHQDIPLIAATGGPGVVTAVLQSGKRGIGAGAGNPPVLVDETADIRKAAEDIINGCTFDNNLPCIAEKEVVAIDAIANELMNYMVKEQGCYAITKEQQEKLTNLVITPKGLNRNCVGKDARTLLGMIGIDVPSNIRCIIFEGEKEHPLISEELMMPILGIVRAKSFDDAVEKAVWLEHGNRHSAHIHSKNVDRITTYAKAIDTAILVKNAPSYAAIGFGGEGFCTFTIASRTGEGLTSASTFTKRRRCVMSDSLCIR

SEQ ID NO: 473 - protein, 470 aa, Fusobacterium sp.
MRGELMELEVKNIEEIVDLIMKKMTESNVAVSYDSKNGVFDDVDVAIAEAKKAQTVLFSSKLELRERIIASIRETMRAHITELSELAVKETGMGRVKDKEQKNRVAIDRTPGLEDLKAFAFSGDDGLTVMEFSPYGVIGAITPSTNPSETVICNSIGMIAAGNAVIFAPHPGAKRTSIRAVELINEAIKKVGGPENLVVTISEPSIENTEKIIANPNIKMLVATGGPGVVKTVMSSGKKAIGAGAGNPPVLVDETADIEKAAKDIIDGCSFDNNLPCTAEKEVIAVDSIVNYLIFEMQKNGAYLLKDKELIEKLVSLVLKNNSPDRKYVGKDAKYILKQLGIEVGDEIRVIIVETDKNHPFAVEELLMPVLPIVKVKDALEGIKVAKELERGLRHTAIIHSKNIDILSKYAREMETTILVKNGPSYAGIGIGGEGHVTFTIAGPTGEGLTSARSFARNRRCVLVGGFSIK

SEQ ID NO: 474 - protein, 462 aa, Clostridium beijerinckii
MSVNEKMVQDIVQEVVAKMQISSDVSGKKGVFSDMNEAIEASKKAQKIVAKMSMDQREAIISKIREKIKENAEILARMGVEETGMGNVGHKILKHQLVAEKTPGTEDITTTAWSGDRGLTLIEMGPFGVIGAITPCTNPSETVLCNTIGMLAGGNTVVFNPHPAAIKTSIYAVNLLNEASVEVGGPENIAVTVEHPTMETSDVMMKHKDIHLIAATGGPGVVTAVLSSGKRGIGAGAGNPPALVDETADIRKAAEDIVNGCTFDNNLPCIAEKEIVAVDSIADELLHYMVNEQGCYMISKEEQDALTEVVLKGGRLNRKCVGRDAKTLLGMIGITVPDNIRCITFEGPKEHPLIAEELMMPILGVVRAKDFDDAVEQAVWLEHGNRHSAHIHSKNVDNITKYAKAIDTAILVKNGPSYAALGFGGEGYCTFTIASRTGEGLTSASTFTKRRRCVMTDSLCIR

SEQ ID NO: 475 - protein, 462 aa, Ruminococcus gnavus
MSVNEKMVQDIVQEVVAKMQISSDVSGKKGVFSDMNEAIEASKKAQKIVAKMSMDQREAIISKIREKIKENAEILARMGVEETGMGNVGHKILKHQLVAEKTPGTEDITTTAWSGDRGLTLIEMGPFGVIGAITPCTNPSETVLCNTIGMLAGGNTVVFNPHPAAIKTSIYAVNLLNEASVEVGGPENIAVTVEHPTMETSDIMMKHKDIHLIAATGGPGVVTAVLSSGKRGIGAGAGNPPALVDETADIRKAAEDIVNGCTFDNNLPCIAEKEIVAVDSIADELLHYMVSEQGCYMISKEEQDALTEVVLKGGRLNRKCVGRDAKTLLGMIGITVPDNIRCITFEGPKEHPLIAEELMMPILGVVRAKDFDDAVEQAVWLEHGNRHSAHIHSKNVDNITKYAKAIDTAILVKNGPSYAALGFGGEGYCTFTIASRTGEGLTSASTFTKRRRCVMTDSLCIR

SEQ ID NO: 476 - protein, 462 aa, Ruminococcus obeum
MPISESMVQDIVQEVMAKMQIADAPTGKHGVFKEMNDAIEAAKKAELIVKKMSMDQREKIITCIRKKIKENAEVMARMGVDETGMGNVGDKILKHHLVADKTPGTEDITTTAWSGDRGLTLIEMGPFGVIGAITPCTNPSETVLCNTIGMLAGGNTVVFNPHPAAIKTSIFAINLVNEASLEAGGPDNIAVTVEKPTLETSNIMMKHKDIPLIAATGGPGVVTAVLSSGKRGIGAGAGNPPAVVDETADIRKAAQDIVNGCTFDNNLPCIAEKEVVAVSSVVDELMHYMLTENDCYLASKEEQDKLTEVVLAGGKLNRKCVGRDAKTLLSMIGVNAPANTRCIIFEGPKEHPLITTELMMPILGVVRARDFDDAVEQAVWLEHGNRHSAHIHSKNIDNITKYAKAIDTAILVKNGPSYSALGFGGEGYCTFTIASRTGEGLTSASTFTKRRRCVMSDSLCIR

SEQ ID NO: 477 - protein, 473 aa, Clostridium saccharolyticum
MEIGAKEIELIVREVLAGIESRGPKLSYIPAQSDNGVFERVEDAIGAAHTAQREWVEHYRVEDRRRIIEAIRMTAKSHAKTLAKLVWEETGMGRFEDKIQKHMAVIEKTPGVECLTTDAISGDEGLMIEEYAPFGVIGAITPSTNPTETIINNTISMIAGGNAVVFNVHPGAKKCCAHCLKLLHQAIVENGGPANLITMQKEPTMEAVTKMTSDPRIRLMVGTGGMPMVNALLRSGKKTIGAGAGNPPVIVDDSADVSLAAREIYRGASFDNNILCLAEKEVFVMEKAADELVNNLVKEGAYLLNPMELNEILKFAMIEKNGSCEVNKKWVGKDAGLFLEAIGVSGHKDVRLLICETDRNHPFVMVEQLMPILPIVRLRTFEECVESAVAAESGNRHTASMFSRNVENMTRFGKVIETTIFTKNGSTLKGVGIGGEGHTTMTIAGPTGEGLTCARSFTRRRRCMLAEGGLRII

SEQ ID NO: 478 - protein, 471 aa, Flavonifractor plautii
MNIDENVVESIVKRVVSQLSTETASAQTCPSGGDWGVFESMNDAVDAAVEAQREYLNRSMHDRACYVQAIRDVVLDQENLEYISRLAVEETGMGGYEYKLIKNRLAAVKTPGIEDLTTDAMSGDDGLTLVEYSPFGVIGSITPTTNPTETIICNSIGMLAAGNAVVFSPHPRAKKVSLHLIQLINKALCKAGAPANLVVTVSAPSIENTNAMMSHPKIRMLVATGGPAIVKTVLSSGKKAIGAGAGNPPVVVDETADIEKAAKDIVDGCSFDNNLPCIAEKEVIAVDSVADYLIFNMKKNGAYEVKDPAVISQLVELVTKEGKSPKTEFVGKSAKYILDKIGITVGDDVKVILMEAKEDHPFVQVELMMPILPLVRVPDVDQAIEMAVRVEHGNRHTAMMHSRNVEKLTKMAKLIQTTIFVKNGPSYAGIGVGGEGYTTFTIAGPTGEGLTSAKSFARRRRCVLVGGMDVR

SEQ ID NO: 479 - protein, 462 aa, Ruminococcus obeum
MPISESMVQDIVQEVMAKMQIADAPTGKHGIFKDMNDAIEAAKKSELIVKKMSMDQREKIITCIRKKIKENAEVMARMGVDETGMGNVGDKILKHHLVADKTPGTEDITTTAWSGDRGLTLIEMGPFGVIGAITPCTNPSETILCNTMGMLAGGNTVVFNPHPAAIKTSIFAINLVNEASLEAGGPDNIAVTVEKPTLETSNIMMKHKDIPLIAATGGPGVVTAVLSSGKRGIGAGAGNPPAVVDETADIRKAAQDIVNGCTFDNNLPCIAEKEVVAVSSVVDELMHYMLTENDCYLASKEEQDKLTEVVLAGGKLNRKCVGRDARTLLSMIGVNAPANIRCIIFEGPKEHPLITTELMMPILGIVRAKDFDDAVEQAVWLEHGNRHSAHIHSKNVDNITKYAKAIDTAILVKNGPSYSALGFGGEGYCTFTIASRTGEGLTSASTFTKRRRCVMSDSLCIR

SEQ ID NO: 480 - protein, 469 aa, Clostridium carboxidivorans
MELQSNELSLIIEKVLKEMNKKELKEEVSDGVFDTMEEAIEAAYEAQKKFSSYTIEQREKLIAAMRKAIIDNAMEIANLCVNESGMGRVDHKYLKLKLTAEKTPGTEVLQTTAFTGDKGLTLVENGAFGVIGSITPSTNPAATVACNGIGMLAGGNTAVFSPHPGAFRSSLAMLRALNKAIKEAGGPDNLLTSVKKPSIESTNSMMKNDKIRMVVATGGPGIVKMVLSSGRKAIGAGAGNPPVVVDETADIKKAARDIIAGCTFDNNLPCIAEKEALVVEAVYEELIKEMKNNRAVYELNDEEAAKVAELVLVHNKEKNTYSINKAFVGKDAKYILQNIGKNDAEGVECLIYRAENSHPFVQEELMMPILPIVKTKDFEEALKLAVQDEHGNRHTAIMHSKNVDNLTKMARAIDTTIFVKNAPSYAGIGFGGEGYTTFTIAGPTGEGLTNAVSFTRKRRCTMAESFRIV

SEQ ID NO: 481 - protein, 469 aa, Fusobacterium ulcerans
MNLEANNMDEIVALIMKELKKTDIKAGCQSCESPKNGVFSSMDEAIAAAKKAQEILFSSRLEMREKIVASIREVMKDYVVELAELGVKETGMGRAADKALKHQVTIEKTPGVEDLRAFAFSGDDGLTVMELSPYGVIGAITPSTNPSETIICNSIGMISAGNSVVFAPHPGAKRTSIKTVEIINEAVRKAGGPENLVVTIAEPSIENTNRMMENPDIKMLVATGGPGVVKSVMSSGKKAIGAGAGNPPVLVDETADIEKAARDIVAGCSFDNNLPCIAEKEVVAVDSITDYLIFEMQKNGAYLIKDKSVIDRLVAMVLKNGSPNRAYVGKDASYILKDLGINVGGEIRVIITEADKDHPFAVEELLMPILPIIRVKNALEGIEVSKKLEHGLRHTAMIHSKNIDILTKYARDMETTILVKNGPSFAGIGVGGEGHTTFTIAGPTGEGLTSAKSFARNRRCVLVGGLSIK

SEQ ID NO: 482 - protein, 469 aa, Fusobacterium sp.
MNLEANNMDEIVALIMKELKKTDIKAGCQSCESLKNGVFSSMDEAIAAAKKAQEILFSSRLEMREKIVASIREVMKDYVVELAELGVKETGMGRAADKALKHQVTIEKTPGVEDLRAFAFSGDDGLTVMELSPYGVIGAITPSTNPSETIICNSIGMISAGNSVVFAPHPGAKRTSIKTVEIINEAVRRAGGPENLVVTIAEPSIENTNRMMENPDIKMLVATGGPGVVKSVMSSGKKAIGAGAGNPPVLVDETADIEKAARDIVAGCSFDNNLPCIAEKEVVAVDSITDYLIFEMQKNGAYLIKDKSVIDRLVAMVLKNGSPNRAYVGKDASYILKDLGINVGDEIRVIITETDKDHPFAVEELLMPILPIIRVKNALEGIEVSKKLEHGLRHTAMIHSKNIDILTKYARDMETTILVKNGPSFAGIGVGGEGHTTFTIAGPTGEGLTSAKSFARNRRCVLVGGLSIK

SEQ ID NO: 483 - protein, 457 aa, Roseburia inulinivorans
MVHDIVQKVMANMQISGSVSGMHGVFKDMNDAINASIEAQKKVCTMTLDQREQIISLIRKKTHENAEILANMGVNETGMGNVGDKILKHHLTADKVPGTEDISTIAWSGDRGLTLVEMGPFGVIGAITPATNPSETVICNCIGMLAGGNTVVFNPHPNAKKTTIYTINMINEASIEAGGPDNIAVTVEAPTLDTSAIMMKHPSIHLLVATGGPGVVTAVLSSGKRAIGAGAGNPPVLVDETADIRKAAQDIVNGCTFDNNLPCIAEKEIVAVDSVADELMNYMISENGCYLASKEIQDKLVQTVFTPKGALNRKCVGRSAQTLLAMVGVNVGPEIRCIVFEGQKEHPLIAEELMMPILGMVRVKSFEEGVETAVWLEHGNRHSAHIHSKNVDHITTYARALDTAILVKNGPSYAALGFGGEGYCTFTIASRTGEGLTAAHSFTKSRRCTMSDSLCIR

SEQ ID NO: 484 - protein, 469 aa, Clostridium carboxidivorans
MELESNELSVIIEKVLKEMNKKEFGKKESDGIFDTMDEAVEASYEAQKKYSSYSLEQREKLIQAMRKAIMDNAMEVANLCVKESGMGRVDHKYLKLKLIVEKTQGTEILRPEVYTGDNGLTLIEHGAFGVIGAITPSTNPAATVACNSICMLAGGNTVVFSPHPGALNSCLTMIRILNKAIKEAGGPENLITSVKAPSIENTNIMINHKRIRLVVATGGPGIVKLVLSSGKKAIGAGAGNPPVVVDETADIPKAARDIIAGCSFDNNLPCIAEKEAIVVESVYEELIKEFKKNRVVYELTDEEAEKLVGKVLNYDEKNKKYSINKKFVGKDAKYLLESIGKDAGTGVECLIYRAENSHPFVQEELMMPILPIVKVKNVDEAIETAVEDEHGNRHTAMMHSKNVVNLTKMARAIDTTIFVKNAPSYAGIGFGGEGHTTFTIAGPTGEGITNAVTFTRQRRCTMVDSFRIV

SEQ ID NO: 485 - protein, 471 aa, Clostridium sp.
  REGION 1..471: note = Clostridium cf. saccharolyticum K10
MEMDMKVIEQLVAQALKEMKAEEPAAFAEKKEENYGVFSTMDEAIEASEKAQKALLFSKIQDRQKYVDIIRAAILKRENLELISRMAVEETEIGKYEHKLIKNRLAAEKTPGTEDLTTEAQTGDHGLTLVEYCPFGVIGAITPTTNPTETIICNSISMIAGGNTVVFSPHPRAKKVSQLLVKMLNKALMEGGAPANLITMVEEPSIENTNKMIEHPGVRLLVATGGPAIVKKVLSSGKKAIGAGAGNPPVVVDETADIEKAARDIVDGCSFDNNVPCIAEKEVFAVDSICDYLIQNMKLNGAYEIRDAETIERLDALVTNEKGGPKTSFVGKSAKYILDKMGIPADDSVKVIIMEVRRDHHLVTEEMMMPILPIVRVSDVDTAIEYAHDAEHGNRHTAMMHSKNVEKLSKMAKLLETTIFVKNAPSYAGIGAGGEGHATFTIAGPTGEGLTSARSFCRKRRCVMSDAFSIR

SEQ ID NO: 486 - protein, 469 aa, Fusobacterium varium
MNLEANNMDEIVALIMKELKKTDIKTVCQSCENPKNGVFSSMDEAITAAKKAQEILFSSRLEMREKIVASIREVMKDYVLELAELGVKETGMGRVADKALKHQVTIEKTPGVEDLKAFAFSGDDGLTVMELSPYGVIGAITPSTNPSETIICNSIGMISAGNSIVFAPHPGAKRTSIKTVEIINEAVRKVGGPENLVVTIAEPSIENTNKMMANPDIKMLVATGGPGVVKSVMSSGKKAIGAGAGNPPVLVDETADIEKAAKDIVAGCSFDNNLPCIAEKEVVAVDSITDYLIFEMQKNGAYLIKDKAVIERLAGMVLKNGSPNRAYVGKDASYILKDLGINVGDEIRVIIAETDKEHPFAVEELLMPILPIIRVKNALEGIEVSKKLEHGLRHTAMIHSKNIDVLTKYARDMETTILVKNGPSFAGIGVGGEGHTTFTIAGPTGEGLTSAKSFARNRRCVLVGGLSIK

SEQ ID NO: 487 - protein, 482 aa, Clostridium celatum
MDDNTKLIQDIVAKVISEIGTKEIEEEACCGNGSCGGSCGCNKEKYVFEDVDSAVAAAKKAYKELKQLTIKDRENIITKIREKCLTYSERLSIMAVDETGMGKVEDKITKHVLVARKTPGTEDLTTTAWSGDGGLTLVERGAFGVIAAITPSTNPTATIFCNSIGMIAAGNSVVFAPHPAAKSCSKFAVKLINEASIEVGGPENIVVTFENPSIEITSALMKHKDIPFISATGGPGVVTQACSSGKRAIGAGAGNPPVLVDETADIKHAAKSIIAGATFDNNLPCIAEKEVVALDSICDELIEDMQKEGAYFLNSTELINRLIDTVLIRKDGKVTLNRNFVGRDAKIILDAIGVYADDSVKCIIFEGCKSNLLIVEELMMPILGIVRVKDFNTAVDVAVELEHGNRHSAHIHSKRIDRLTYFAREIDTAIFVKNAPSYSALGVEAEGYPTFTIASRTGEGLSSAKTFSKSRRCIMKDALSIK

SEQ ID NO: 488 - protein, 462 aa, Clostridium sp.
MSVNERMVQDIVQEVVAKMQIASDVTGNHGVFQDMNAAIEAAKKTQKVVARMSMDQREKIISNIRAKIKEHAEIFARMGVQETGMGNVGHKILKHQLVAEKTPGTEDIQTTAWSGDRGLTLIEMGPFGVIGAITPCTNPSETVLCNTIGMLAGGNTVVFNPHPAAIKTSIYAVNLINEASLEAGGPDNIACTVENPTLESSNIMMKHKDIPLIAATGGPGVVTAVLSSGKRGIGAGAGNPPALVDETADIRKAAEDIVNGCTFDNNLPCIAEKEIVAVDSIADELMHYMISEQGCYLASKEEQDALTEVVLKGGRLNRKCVGRDAKTLLGMIGVTVPDNIRCITFEGPKEHPLIAEELMMPILGVVRAKDFDDAVEQAVWLEHGNRHSAHIHSKNVDNITKYAKAIDTAILVKNGPSYAAIGFGGEGFCTFTIASRTGEGLTSASAFTKRRRCVMCDSLCIR

SEQ ID NO: 489 - protein, 462 aa, Clostridium beijerinckii
MSVNEQMVQDIVQEVMAKMQITSDVSGSHGVFKDMNEAIAAAKKTQKIVGKMSMDQREKIISNIRTKIKENAEIMARMGVQETGMGNVGHKILKHVLVAEKTPGTEDITTTAWSGDRGLTLIEMGPFGVIGAITPCTNPSETVLCNTIGMLAGGNTVVFNPHPAAIKTSIFAINLLNEASLEAGGPDNIACTVEKPTLASSDIMMKHKDIPLIAATGGPGVVTAVLSSGKRGIGAGAGNPPALVDETADIRKAAEDIVNGCTFDNNLPCIAEKEIVAVDSIADELMYYMVSEQGCYKITKEEQDALTAVVLKDGKLNRKCVGRDAKTLLGMIGVTVPDNIRCITFEGPKEHPLIAEELMMPILGVVRAKDFDDAVEQAVWLEHGNRHSAHIHSKNVDNITTYAKAIDTAILVKNGPSYAALGFGGEGYCTFTIASRTGEGLTSASTFTKRRRCVMTDSLCIR

SEQ ID NO: 490 - protein, 463 aa, Clostridium beijerinckii
MPVSESMVQDIVKEVVARMQLSGSAGTAQHGVFTDMNQAIEAAKEAEAKVRCMTMDQREQIVSNIRRKTHENAELLARMGVEETGMGNVGDKILKHHLLADKTPGTEDITTTAWSGDRGLTLIEMGPFGVIGAITPCTNPSETVLCNSMGMIAAGNTVVFNPHPQAIKTSIFAINMVNEASLEAGGPDNVACTVSKPTLETSNIMMKHKDIPLIAATGGPGVVTAVLSSGKRGIGAGAGNPPALVDETADVRKAAADIVNGCTFDNNLPCIAEKEIVAVDSVADELMNYMISEQGCYLISKEEQDKLTATVITPKGLNRKCVGRDARTLLSMIGIQAPENIRCIVFEGEKEHPLIAEELMMPILGLVRAKDFDDAVEKAVWLEHGNRHSAHIHSKNVDNITKYAKAIDTAILVKNAPSYAALGFGGEGFCTFTIASRTGEGLTSASTFTKRRRCVMSDSLCIR

SEQ ID NO: 491 - protein, 462 aa, Ruminococcus sp.
MPINENMVQEIVQEVMAKMQIADAPTGKHGIFKEMNDAIEAAKKSQLIVKKMSMDQREKIITCIRKKIKENAEVMARMGVEETGMGNVGDKILKHHLVADKTPGTEVITTTAWSGDRGLTLIEMGPFGVIGAITPCTNPSETILCNTMGMLAGGNTVVFNPHPAAIKTSIYAINLLNEASLESGGPDNIAVTVEKPTLETSNVMMKHKDIPLIAATGGPGVVTAVLSSGKRGIGAGAGNPPALVDETADIRKAATDIVNGCTFDNNLPCIAEKEIVAVSSIVDELMHYLVTENDCYLASKEEQDKLTEVVLAGGKLNRKCVGRDARTLLSMIGVNAPANIRCIVFEGPKEHPLITTELMMPILGVVRARDFDDAVEQAVWLEHGNRHSAHIHSKNIDNITKYAKAIDTAILVKNAPSYAALGFGGEGYCTFTIASRTGEGLTCASTFTKRRRCVMADSLCIR

SEQ ID NO: 492 - protein, 469 aa, Acetobacterium woodii
MNIDTTGIEYIVKKVMAEIDCAEEGGKPLKDGELGIFNDMENAIDAAFIAQKSFMRASMAFRSKIIAAMRAEMLKKENMEMICQMAVEETGMGNYEHKLLKHELAATKTPGVEDLVADAFTGDDGLTLIEQSPFGVIGAVSPSTNPSETIICNGIGMLAGGNTVVFAPHPSAKKTSALVVKLLNKAILEAGGPENLIVTTVKPTIDSANTMFASPKITMLCATGGPGVVKSVLQSGKKAIGAGAGNPPALVDETADIEKAGKDIIDGCCFDNNLPCIAEKEVVVVEQVADYLIFNMKKNGAYELKDAQKIKELEELVIPGGRLSRDYVGRSAKVILKGIGIEVDDSVRVVIIETSKDHIFAVEELMMPILAIVRVKDVAEGIDLAVSLEHGNRHTAIMHSTNINNLTEMAKRVQTTIFVKNGPSYAGIGVGGEGYTTFTIAGPTGEGLTSAKTFTRKRRCVLVGGFTIK

SEQ ID NO: 493 - protein, 497 aa, Clostridium botulinum
MNDFNMIDIESIVKNIVKELTGNEKGQGAITTATAPKEANPLVDIEKKIMGFMNTPTMPVGEYGVFEDINDAIEQAWLAEQEYRKVGLDKRTEIIEAFKAEVRKNVEEISRRTFEETGMGRYEDKILKNNLALDKTPGVEDLEAGVKTGDGGLTLYEMSPFGVIGAIAPSTNPTETIINNGISMLAGGNTVVFSPHPGAKDVSVFIVQLINKAIERINGPKNLIVTVKNPNIESTNIMLAHPKVNMICATGGPGIVKVALSSGKKAIGAGAGNPPVVVDETADIEKAAVDIIDGCSFDNNLPCICEKEVIVVDKVADYLKTCMSKYCALEITDKNMLAQLEKLVLTENGTINKQFVGKNADYIMSKLGVNIDPSIRVIFAEVEANHPFAVEELMMPILPVIRVRNVDEAIDLGVELEHGNRHTAIMHSKHIDNLSKFAKAVQTTIFVKNAPSYAGIGYGAEGHGTFTIAGPTGEGLTSARTFTRKRRCVMVDNFSIK

SEQ ID NO: 494 - protein, 497 aa, Clostridium botulinum
MNDFNMIDIESIVKNIVKELTGNEKEQGAIITATAPKEVNPLVDIEKKIMGFMNTPTMQAGEYGVFEDINDAIEQAWLAEQEYRKVGLDKRTEIIEVFKAEVRKNVEEISRRTFEETGMGRYEDKILKNNLALDKTPGVEDLEAGVKTGDGGLTLYEMSPFGVIGAIAPSTNPTETIINNGISMLAGGNTVVFSPHPGAKDVSVFIIQLINKAIERVNGPKNLIVTVRNPNIESTNIMLSHPKVNMICATGGPGIVKVALSSGKKAVGAGAGNPPVVVDETADIEKAAVDIIDGCSFDNNLPCICEKEVIVVDKVTDYLKTCMSKYCALEITDKNMLAQLEKLVLTENGTINKKFVGKNADYIMSKLGINIDPSIRVIFAEVGANHPFAVEELMMPILPIIRVRNVDEAIELGVELEHGNRHTAIMHSKHIDNLSKFAKAVQTTIFVKNAPSYAGIGYGAEGHGTFTIAGPTGEGLTSARTFTRKRRCVMVDNFSIK

SEQ ID NO: 495 - protein, 497 aa, Clostridium botulinum
MNDFNMIDIESIVKNIVKELTGNEKEQGTITTAAVPKEVNPLVDIEKKIMGFVNTPTMPIGEHGVFEDINDAIEQAWIAEQEYRKVGLDKRTEIIEAFKAEVRKNVEEISRRTFEETGMGRYEDKILKNNLALDKTPGVEDLEAGVKTGDGGLTLYEMSPFGVIGAIAPSTNPTETIINNGISMLAGGNTVVFSPHPGAKDVSVFIIQLINKAIERVNGPKNLIVTVRNPNIESTNIMLAHPKVNMICATGGPGIVKVALSSGKKAIGAGAGNPPVVVDETADIEKAAVDIIDGCSFDNNLPCICEKEVIVVDKVADYLKTCMSKYCALEITDKNMLAQLEKLVLTENGTINKKFVGKNADYIMSKLGVNIDPSIRVIFAEVEANHPFAVEELMMPILPVIRVRNVDEAIDLGVELEHGNRHTAIMHSKHIDNLSKFAKAVQTTIFVKNAPSYAGIGYGAEGHGTFTIAGPTGEGLTSARTFTRKRRCVMVDNFSIK

SEQ ID NO: 496 - protein, 462 aa, Eubacterium plexicaudatum
MSVNDQMVQDIVRQVLANMRISSDASGSRGVFSDMNEAVEAAKKAQAVIGKMPMDHREKIISSIRAKIMENAEILARMGVKETGMGNVGHKILKHQLVAEKTPGTEDITTKAWSGDRGLTLIEMGPFGVIGAITPCTNPSETILCNTIGMVAGGNTVVFNPHPAAIKTSIFAVNLVNEASVEAGGPDNIACTVEHPTLDTSAIMMKHKDIHLIAATGGPGVVTAVLSSGKRGIGAGAGNPPALVDETADIRKAAEDIVNGCTFDNNLPCIAEKEIVAVDSIADELMHYMISEQGCYLASAKEQEALISVVLKGGQLNRDCVGRDAKTLLGMIGVQAPDNIRCITFEGPKEHPLITEELMMPILGVVRADSFEDAVEKAVWLEHGNRHSAHIHSKNVDHITTYAKAIDTAILVKNGPSYAAIGFGGEGYCTFTIASRTGEGLTSASAFTKRRRCVMCDSLCIR

SEQ ID NO: 497 - protein, 467 aa, Thermosediminibacter oceani
MVDEKVVEAIAKRIIEELNLCESGSSGGESREELGIFDNLDDAVEAASQAQKRFAALDLEKREEIIQAIREACLNNARYLAELTVNETGIGRVEDKIVKNILAAKKTPGTEDLRPSCWTGDHGLTLVEMAPVGVIGSITPVTNPVATVINNSISMLAAGNAVVFNPHPSAKRSSNKAVEIINEAIMKVGGPRHLVNSVAEPTIETAKALMAHPKVNLVSVTGGKAVVSEALRSGKKVIGAGAGNPPVVVDETADIVKAAHDIYCGASFDNNLPCIAEKELIAVEAVADMLLERLAREGAYILRGKDVEKITEVVFDENHRINKKLVGKDASFILEQIGIQVGKDVRLVVVPVNPEHPLVHHEQLMPVLPFVRVPNIQEAVELAVRAEGGNRHTAVMHSKNVDNMTNFARAIQTTIFVKNAPSYAGIGFGGEGYATFTIAGPTGEGLTSARTFTRQRRCVLVDAFRII

SEQ ID NO: 498 - protein, 479 aa, Clostridium clostridioforme
MEISEKEVEAIVRSVLSGLGQKSFQAEALHVKDKMCSDGEDGIFELVEDAIEAASKAQKEWVHRYKLKDRKRIIEAIRVTSRAHAESLARMVHEETGMGRYEDKITKHMAVIDKTPGVECLVTDAISGDEGLMIEEPAPFGVIGAITPSTNPTETMINNTISMIAGGNAVVFNVHPGAKKCCAYCLQILHRAIVENGGPKNLITMQREPDMDAVHKLTSSPHIRLMVGTGGMGMVHALLCSGKRTIGAGAGNPPVVVDDTADLSLAARELYRGASFDNNLLCLAEKEVFVMDNVAEELVDRLVGEGAYLLDDLQLKKITELAMVNKDGKYEVNKKWVGKDAGKFLEAIGIQEHREPRLLICVTDRSHPFVKVEQLMPVLPIVRCGSFEKCVEWAVDTEAGNRHTASIFSKNVEHMTLFGKEIETTIYTKNGATLKGIGIGGEGHTTMTIAGPTGEGLTCARSFTRRRRCMLAEGGLRII

SEQ ID NO: 499 - protein, 471 aa, Clostridium clostridioforme
MDMDIKVIEQMVEQALKEIKAEQPQKFTMPKAELYGVFKTMDEAIAASEEAQKKLLFSKISDRQKYVDVIRRTILKRENLEMISRLSVEETEIGDYEHKLIKNRLAAEKTPGTEDLLTEAMTGDNGLTLVEYCPFGVIGAITPTTNPTETIINNSISMIAGGNTVVFSPHPRAKKVSQMTVKLLNKALTESGAPENLITMVEEPSIENTNKMIENPSVRLLVATGGPSIVKKVLSSGKKAIGAGAGNPPVVVDETADIVKAAKDIVDGCSFDNNVPCIAEKEVFAVDSICDYLIHNMKENGAYQITDPALLEKLVTLVTNEKGGPKTSFVGKSARYILDKLGITADASVRVIIMEVPKEHLLVQEEMMMPILPVVRVCDVDTAIEYARQAEHGNRHTAMMHSRNVEKLSKMAKIMETTIFVKNAPSYAGIGVGGEGYTTFTIAGPTGEGLTSPRAFCRKRKCVMTDAFSIR

SEQ ID NO: 500 - protein, 472 aa, Ilyobacter polytropus
MNLDANNLNNIVSLIMKELDKNNNIDDTGQGCGGEEGKNGIFSSMDTAVSKAKEAQVTLFASKLELRERIIKAIREDVREAAAELAEIAVEETGMGRVDDKTLKHYVTVDKTPGVEDLRAFAYSGDNGLTVMELSPYGVIGSITPSTNPSETIVCNAIGMIAAGNSVVFAPHPGAKKTSLRAVEILNKAVARAGGPNNLVVTIFEPSIENTNKMVKNPDIKMVVATGGPGVVKSVMSSGKKAIGAGAGNPPVLVDETADIEKAAKDIVNGCSFDNNLPCITEKEVVAVDSITDYLIFEMQKNGAYLVQDSKTIKKLCEMVINDGSPNRAYVGKNASYILKDLGIDVGDEIKVIIVETDAGHPLAVLEMLMPVLPIVRVKDALEGIKVCKKLEDGLRHTAMIHSKNIDILTKYARDMETTILVKNGPSYSGIGVGGEGYTTFTIAGPTGEGLTSAKSFARNRRCALVGGLSIK

SEQ ID NO: 501 - protein, 462 aa, Shuttleworthia satelles
MADEQLVQNVVREVVARMQISAPARGMHGVFSDMEEAIEAARTAQQTVRLLPMDQREKIIGAIRRKTRENAEILARMAVNETGMGNVGDKILKHLLVADKVPGTEDISTRAFSGDRGLTLIEMGPFGVIGAITPCTNPSETVLCNTIGMLAGGNTVVFNPHPQAIKTTLFTIQMVNEASLEAGGPDNIACTVDAPTLATSEIMMKSPHIKLLVATGGPGVVTAVLSSGKRAIGAGAGNPPALVDETADIRKAAEDIVNGCTFDNNLPCIAEKEIVAVDSIADELLHYMLTEQGCYQASEEELDRLTKAVMDEKGRLNRKAVGRSARKLLSMIGVEVDANIRCITFFGPKEHPLITTELMMPILGIVRVKDFAEGLETAAWLEHGNKHSAHIHSKNVDRITEYARRLDTTITVKNGPSYAALGFGGESYCTFTIASRTGEGLTSARSFIKSRHCVMTDSLCVR

SEQ ID NO: 502 - protein, 472 aa, Clostridium beijerinckii
MDVDVVLVEKLVRQAIEEVKNKNLLNLDKFESVKNYGIFGTMDAAVEASFVAQKQLLNASMTDKQKYVDTIKATILKKENLELISRMSVEETEIGKYEHKLIKNRVAAEKTPGIEDLTTEAMTGDNGLTLVEYCPFGVIGAITPTTNPTETIICNSISMIAGGNTVVFSPHPRAKNVSIKLVTMLNKALEEAGAPDNLIATVKEPSIENTNIMMEHPKIRMLVATGGPAIVNKVMSTGKKAIGAGAGNPPVVVDETADIEKAAIDIVNGCSFDNNVPCIAEKEVFAVDQICDYLIHYMKLNGAYEIKDRDLIQKLLDLVTNENGGPKVSFVGKSAPYILNKLGISVDENIKVIIMEVEKNHHFVLEEMMMPILPIVRTKDVDEAIECAYVAEHGNRHTAIMHSKNVDKLTKMARLLETTIFVKNAPSYAGIGVGGEGTTTFTIAGPTGEGLTTARSFCRKRRCVMVDAFNIR

SEQ ID NO: 503 - protein, 471 aa, Clostridium clostridioforme
MDMDIKVIEQMVEQALKEIKAEQPQKFTMPKAELYGVFKTMDEAIAASEEAQKKLLFSKISDRQKYVDVIRRTILKRENLEMISRLSVEETEIGDYEHKLIKNRLAAEKTPGTEDLLTEAMTGDNGLTLVEYCPFGVIGAITPTTNPTETIINNSISMIAGGNTVVFSPHPRAKKVSQMTVKLLNKALTESGAPENLITMVEEPSIENTNKMIENPSVRLLVATGGPSIVKKVLSSGKKAIGAGAGNPPVVVDETADIVKAAKDIVDGCSFDNNVPCIAEKEVFAVDSICDYLIHNMKENGAYQITDPALLEKLVTLVTNEKGGPKTSFVGKSARYILDKLGITADASVRVIIMEVPKEHLLVQEEMMMPILPVVRVCDVDTAIEYARQAEHGNRHTAMMHSRNVEKLSKMAKIMETTIFVKNAPSYAGIGVGGEGYTTFTIAGPTGEGLTSPKAFCRKRKCVMTDAFSIR

SEQ ID NO: 504 - protein, 473 aa, Clostridium sp.
MKLDDKLIEQVARLVMEEMKSGSAAACEENGTCGDSYGIFDSMDDAVQASEAAQRKYLFSTMEDRQKYVDVIRQTVLEPEMLQKISRMAVEETGMGNYEHKLIKNRLAAEKSPGTEDLVTEAMTGDRGLTLVEYCPFGVIGAVTPATNPTETIICNSIAMLAGGNTVVFSPHPRAKNVTHVLVTALNQALEKVGAPTNLIVTVREPSVENTNLMIKHPKIRVLVATGGPGIVKMVMSTGKKAIGAGAGNPPVVVDETADIEKAAKDIVDGCSFDNNLPCIAEKEVIAVDTIADCLIWHMKRVGAFELKEESAISRLLQLVTNEKGGPKVEFVGKSAPYILNKLGISGGENARVILMETQKDHPFVMEELMMPILPIVRAADVDEAIEIALVAERGNRHTAMMHSKNVDKLTKMAKLLQTTIFVKNAPSYAGIGVGGEGCTTFTIAGPTGEGLTTARSFCRKRRCVMSDALHIR

SEQ ID NO: 505 - protein, 478 aa, Clostridium bolteae
MKEGVIRLDMDIKVIEQLVEQALKEIKAEQPLKFTAPKLERYGVFKTMDEAIAASEEAQKKLLFSKISDRQKYVDVIRSTIIKRENLELISRLSVEETEIGDYEHKLIKNRLAAEKTPGTEDLLTEAITGDNGLTLVEYCPFGVIGAITPTTNPTETIINNSISMIAGGNTVVFSPHPRAKKVSQMTVKMLNKALIDNGAPPNLITMVEEPSIENTNKMIDNPSVRLLVATGGPSIVKKVLSSGKKAIGAGAGNPPVVVDETADIDKAAKDIVDGCSFDNNVPCIAEKEVFAVDSICDYLIHHMKENGAYQITDPMLLEQLVALVTTEKGGPKTSFVGKSARYILDKLGITVDASVRVIIMEVPKDHLLVQEEMMMPILPVVRVSDVDTAIEYAHQAEHGNRHTAMMHSKNVEKLSKMAKIMETTIFVKNAPSYAGIGVGGEGYTTFTIAGPTGEGLTSPRTFCRKRKCVMTDAFSIR

SEQ ID NO: 506 - protein, 471 aa, Clostridium bolteae
MDMDIKVIEQLVEQALKEIKAEQPLKFTAPKLERYGVFKTMDEAIAASEEAQKKLLFSKISDRQKYVDVIRSTIIKRENLELISRLSVEETEIGDYEHKLIKNRLAAEKTPGTEDLLTEAITGDNGLTLVEYCPFGVIGAITPTTNPTETIINNSISMIAGGNTVVFSPHPRAKKVSQMTVKMLNKALIDNGAPPNLITMVEEPSIENTNKMIDNPSVRLLVATGGPSIVKKVLSSGKKAIGAGAGNPPVVVDETADIDKAAKDIVDGCSFDNNVPCIAEKEVFAVDSICDYLIHHMKENGAYQITDPMLLEQLVALVTTEKGGPKTSFVGKSARYILDKLGITVDASVRVIIMEVPKDHLLVQEEMMMPILPVVRVSDVDTAIEYAHQAEHGNRHTAMMHSKNVEKLSKMAKIMETTIFVKNAPSYAGIGVGGEGYTTFTIAGPTGEGLTSPRTFCRKRKCVMTDAFSIR

SEQ ID NO: 507 - protein, 468 aa, Eubacterium hallii
MNIDVELIEKVVKKVLNDVETGSSESEYGYGIFDTMDEAIEASAKAQKEYMNHSMADRQRYVEGIREVVCTKENLEYMSKLAVEESGMGAYEYKVIKNRLAAVKSPGVEDLTTEALSGDDGLTLVEYCPFGVIGAIAPTTNPTETVICNSIAMLAGGNTVVFSPHPRSKGVSIWLIKKLNAKLEELGAPRNLIVTVKEPSIENTNIMMNHPKVRMLVATGGPGIVKAVMSTGKKAIGAGAGNPPVVVDETADIEKAAKDIVNGCSFDNNLPCIAEKEVIAVDQIADYLIFNMKNNGAYEVKDPEIIEKMVDLVTKDRKKPAVNFVGKSAQYILDKVGIKVGPEVKCIIMEAPKDHPFVQIELMMPILPIVRVPNVDEAIDFAVEVEHGNRHTAMMHSKNVDKLTKMAKEIETTIFVKNGPSYAGIGVGGMGYTTFTIAGPTGEGLTSAKSFCRKRRCVLQDGLHIRMK

SEQ ID NO: 508 - protein, 467 aa, Halanaerobium saccharolyticum
MKIKENELDKIVNQVISSLNNKQNSNDFNTKINYGIFSTMDEAIAEAVKAQACLQLNYSTEAREKIIKSIRKNVSKHVEKISEMAVEETDMGRIEDKIIKNNLAINKTPGTEDLRTEAFSGKKGLTIVEEAPFGVICSIAPVTNPTETIISNAISMIASCNGVVFNSHPGAKKVSKYIIEVLNKVIMEAGGPENLLTAVNEPTLQTVESCMRDDRIAMIVATGGPGVVNAALSSGKKAIGAGAGNPPVLVDDTVDLKRVAKDIINGASFDNNLPCTSEKAIVALESIADSLLNEMTNQNAQLVHDIKALERVILNDDGSINKALVGKDAAFILNKAGLKAKSEDLRLVIVDVDLRHPFVQKEQLMPVIPLVRAKNFNEAMEMGVDIEEGNRHTAIIHSKNVDNLTKFAKKIETTIYVKNAPSYAGIGAGGEGYATFTIAGPTGEGLTSARSFTRKRRCVLVDGFSII

SEQ ID NO: 509 - protein, 469 aa, Eubacterium limosum
MNIDTTGIEYIVKKVMDQIDYAEETGAPVVDGKDGVFQTMDAAIEAAAVAQKEYMKKPLALRRQMIAAMREIMLKKENIETICAMVVEESGMGNYEHKLAKHRLATTGTPGVEDLLTEAWAGDDGCTLLELSPFGVIGAITPTTNPNETIVNNSIGMLAAGNAVVFSPHPKALKTSFLCIKLLNEAIVSVGGPRNLIVTCANPTIEAANEMMVHPKIRMLVATGGPGVVKAVLSSGKKAIGAGAGNPPALVDETADIEKAAKDIIDGCSFDNNLPCIAEKEVVVVDQVADYLIFNMKKNGAYEITDKKAIDALADLVCPEGRLSRDFVGKSAKYIAAAAGLDVPEDTRVLICETSKDHLLAVEELMMPILPIVRVANVDEGIDVAVELEHGNRHTAIMHSKNVDKLTEMAKRIQTTIFVKNGPSYAGIGFGGEGYPTFTIAGPTGEGLTSAKSFARRRRCVLVGGFDIK

SEQ ID NO: 510 - protein, 466 aa, Thermoanaerobacter sp.
MIDENLVVTITKKILNEINLKEAEEKKEKDNPDLGIFNDVNEAVECAKEAQKKFALMDLEKREEIIAAIREACVNNARLLAEIACSETGRGRVEDKVAKNILAAKKTPGTEDLKPTAWTGDRGLTLVEMAPVGVIASITPVTNPTATIINNTISMLAAGNAVVFNPHPSAKKTSNKAVEIINEAILKVGAPNGLVCSINNPTIQTAQKLMEHPEVNMVVVTGGKAVVQTALRCGKKVIGAGAGNPPVVVDETADIVKAAHDIACGASFDNNLPCIAEKEIIAVERIADTLLERMKREGAYVLHGKDIDRMTELIFQGGAINKDLIGRDAHFILSQIGIETGKDIRLVVMPVDVSHPLVYHEQLMPVIPFVTVPTVEEAINLAVKAEGGNRHTAMMHSKNVENMTAFARAIQTTIFVKNAPSYAGIGFGGEGYTTFTIAGPTGEGLTSARTFTRQRRCVLVDAFRIV

SEQ ID NO: 511 - protein, 529 aa, Rhodospirillum rubrum
MNDGQIAAAVAKVLEAYGVPADPSAAAPAPAAPVAPAAPTAGSVSEMIARGIAKASSDDQIAQIVAKVVGDYSAQAAKPAVVPGAAASTEAGDGVFDTMDAAVDAAVLAQQQYLLCSMTDRQRFVDGIREVILQKDTLELISRMAAEETGMGNYEHKLIKNRLAAEKTPGTEDLTTEAFSGDDGLTLVEYSPFGAIGAVAPTTNPTETIICNSIGMLAAGNSVIFSPHPRATKVSLLTVKLINQKLACLGAPANLVVTVSKPSVENTNAMMAHPKIRMLVATGGPGIVKAVMSTGKKAIGAGAGNPPVVVDETADIEKAALDIINGCSFDNNLPCIAEKEIIAVAQIADYLIFSMKKQGAYQITDPAVLRKLQDLVLTAKGGPQTSCVGKSAVWLLNKIGIEVDSSVKVILMEVPKEHPFVQEELMMPILPLVRVSDVDEAIAVAIEVEHGNRHTAIMHSTNVRKLTKMAKLIQTTIFVKNGPSYAGLGVGGEGYTTFTIAGPTGEGLTSAKSFARKRKCVMVEALNIR

SEQ ID NO: 512 - protein, 466 aa, Eubacterium yurii
MNPELLEDVVRQVLSEMKLESSKMVDIYNYGIFDSVDDAINASEIAQRQLFECSVQKRNEYVNAIRQIILKKDNLEMMSRDAVEETGIGRYEDKILKNKLAAEKTPGMEDLITRAVSGQDGLTLEEYCPFGVIGSITPTTNPTETFISNSISMIVGGNTVVFSPHPRAKNTSIKLVKLMNKALEQVGAPRNLISMVKEPSIENTNLMMNHPKIKMLVATGGPAIVKTVLSSGKKAIGAGAGNPPVVVDETADIEKAAKDIVAGSSFDNNVPCIAEKEVFAVESICDQLIYHMKKNGAYEITSYEMIEKLDKLVSQENGKPNTDFVGKSAKYILEKLGISVDDSIRLIICRTNKDHHLVQEEMLMPILPIVSVSDVDVAIEYAYEAEHRNRHTAIMHSRNVEKLSKMAKKLEATIFVKNAPSYAGIGVGGEGYTTFTIAGPTGEGLTSPKSFCRVRRCTMSDSFSIR

SEQ ID NO: 513 - protein, 466 aa, Eubacterium sp.
MNPELLEDVVRQVLSEMKLESSKMVDIYNYGIFDSVDDAINASEIAQRQLFECSVQKRNEYVNAIRQIILKKDNLEMMSRDAVEETGIGRYEDKILKNKLAAEKTPGMEDLITRAVSGQDGLTLEEYCPFGVIGSITPTTNPTETFISNSISMIVGGNTVVFSPHPRAKNTSIKLVKLMNKALEQVGAPRNLISMVKEPSIENTNLMMNHPKIKMLVATGGPAIVKTVLSSGKKAIGAGAGNPPVVVDETADIEKAAKDIVAGSSFDNNVPCIAEKEVFAVESICDQLIYHMKKNGAYEITSYEMIEKLDKLVSQENGKPNTDFVGKSAKYILEKLGINVDDSIRLIICRTNKDHHLVQEEMLMPILPIVSVSDVDVAIEYAYEAEHRNRHTAIMHSRNVEKLSKMAKKLEATIFVKNAPSYAGIGVGGEGYTTFTIAGPTGEGLTSPKSFCRVRRCTMSDSFSIR

SEQ ID NO: 514 - protein, 532 aa, Vibrio sp.
MNEQEIAHAVENVLSKYTNVTAQNAEPVSYSSNASLENIVSQALAGNMVKQPETQTAPDLNSNIENIVSQILAENQAKPQSVQCQSANHGTTEYLGCFASMEEAISAASHAQVQYRHCTMGDRASFVKGIREVFTQDDVLEKISRMAVEETGMGNYADKLTKNRIAATKTPGIEDLTTSALSGDSGLTLTEFSAYGVIGSITPTTNPTETIINNSIGMLAAGNTVVYSPHPRSRNVSLVAVDLINRKLAELGAPANLVVTVLEPSIDNTNAMMNDPRVNMLVATGGPSIVKTVMSTGKKAIGAGAGNPPAVVDETANIEKAAKDIINGCAFDNNLPCIAEKEVIVVNEVADYLIHCMKKSGAYLLCDKQKIQQLQSLVLNEKGTGPNTSFVGKGARYILDKLNIQVSDDIKVILIETERNHPFVVHELMMPILPVVRVENVDEAIDLAIKVEHGNRHTAIMHSTNVEKLSKMARLIQTTIFVKNGPSYSGIGVGGEGHTTFTIAGPTGEGITSARSFARYRRCVMVEALNIR

SEQ ID NO: 515 - protein, 467 aa, Clostridium beijerinckii
MNAELLQDVVRQVLSEMKLESSNILSNEYNYGIFDDMEAAINASETAQRKLFECSVQQRNEFANVIRKEILKKDNLEMISRDAVEETEIGRFEDKILKNKVAAEKTPGMEDLTTRAISGKDGLMIEEYCPFGVIGSITPTTNPTETLINNSISMIVGGNTVVFSPHPRAKNVSIKLVKMMNKALEEHGAPRNMITMVKEPSIENTNLMMSNPKVKLLVATGGPFIVNTVLSSGKKAIGAGAGNPPVVVDETADIEKAAIDIVSGASFDNNVPCIAEKEVFAVDSISDMLIYHMKKNGAYEIVSQDMIEKLDKLVSQENGKPKTEFVGKSAKYILEKLGIYVDDSIRLIICRTSKNHHLVQEEMLMPILPIVSVSDVDIAIEYAYEAEHGNRHTAIMHSKNVEKLSKMAKKLEATIFVKNAPSYSGIGVGGEGHTTFTIAGPTGEGITSAKSFCRIRRCVMHDSFSIR

SEQ ID NO: 516 - protein, 473 aa, Propionibacterium propionicum
MKIDPAQLEATIREVLAAMLPGNDNQTEAPATQQEAPGDGVFADMDSAVEAAHLAQREYLSHPMADRRRYVAAIREAMLAPEALDYMSEQAVAQSGMGDVGHKYLKNKVAAAETPGVEDLVTEAWSGDDGLTTIEYSPYGVIGAITPTTNPTETITCNSIGMLAAGNAVVFSPHPRVAKLSCWQVRRINRALRAAGAPDNLVVTVTAPSLENTNAMMAHPKVRMLVATGGPGIVKAVLSSGKKAIGAGAGNPPAVVDETADIEHAAKCIVDGASFDNNLPCTAEKEIIAVDSIADMLKFCMIKHGAYEATASEVAELEKLLVNGDKPRTEWVGKPAAKILEAIGVTPPPGVRLIVCEASATHPFVVHELMMPVLGLVRVPDVDAAIDLAVELEHGNRHTAVMHSLNVSKLTKMGKLIQTTIFVKNGPSYNGIGIGGEGYPTFTIAGPTGEGLTSARSFTRKRRCVLVGDLNVR

SEQ ID NO: 517 - protein, 467 aa, Clostridium beijerinckii
MNAELLQDVVRQVLSEMKLESSNILSNEYNYGIFDDMEAAINASETAQRKLFECSVQQRNEFANVIRKEILKKDNLEMISRDAVEETEIGRFEDKILKNKVAAEKTPGMEDLTTRALTGKDGLMIEEYCPFGVIGSITPTTNPTETLINNSISMIVGGNTVVFSPHPRAKNVSIKLVKMMNKALEEYGAPRNMITMVKEPSIENTNLMMSNPKVKLLVATGGPFIVNTVLSSGKKAIGAGAGNPPVVVDETADIEKAAIDIVSGASFDNNVPCIAEKEVFAVDSISDMLIYHMKKNGAYEIVSQDMIEKLDKLVSQENGKPKTEFVGKSAKYILEKLGIYVDDSIRLIICRTSKNHHLVQEEMLMPILPIVSVSDVDIAIEYAYEAEHGNRHTAIMHSKNVEKLSKMAKKLEATIFVKNAPSYSGIGVGGEGHTTFTIAGPTGEGITSAKSFCRIRRCVMHDSFSIR

SEQ ID NO: 518 - protein, 467 aa, Clostridium beijerinckii
MNAELLQDVVRQVLSEMKLESSNILSNEYNYGIFDDMEAAINASETAQRKLFECSVQQRNEFANVIRREVLKKDNLEMISRDAVEETEIGRFEDKILKNKVAAEKTPGMEDLTTRALTGKDGLMIEEYCPFGVIGSITPTTNPTETLINNSISMIVGGNTVVFSPHPRAKNVSIKLVKMMNKALEEYGAPRNMITMVKEPSIENTNLMMSNPKVKLLVATGGPFIVNTVLSSGKKAIGAGAGNPPVVVDETADIEKAAIDIVSGASFDNNVPCIAEKEVFAVDSISDMLIYHMKKNGAYEIVSQDMIEKLDKLVSQENGKPKTEFVGKSAKYILEKLGIYVDDSIRLIICRTSKNHHLVQEEMLMPILPIVSVSDVDIAIEYAYEAEHGNRHTAIMHSKNVEKLSKMAKKLEATIFVKNAPSYSGIGVGGEGHTTFTIAGPTGEGITSAKSFCRIRRCVMHDSFSIR

SEQ ID NO: 519 - DNA, 5097 bp, synthetic construct
  misc_feature 1..5097: note = Description of Artificial Sequence: Synthetic polynucleotide
  source 1..5097: mol_type = other DNA
tgtgagtgaaagtcacctgccttaatatctcaaaactcatcttcgggtgacgaaatatggcgtgactcgatacttcactccgcaatgcattccttgatgaattcgcaggaccgtgatacacgggacaggtcactgaatgacgacaatgtcctggaaatcagcgaaccgcgcatctgaagtacatttgagcgactgtaccagaacatgaatgaggcgtttggattaggcgattattagcagggctaagcattttactattattattttccggttgagggatatagagctatcgacaacaaccggaaaaagtttacgtctatattgctgaaggtacaggcgtttccataactatttgctcgcgttttttactcaagaagaaaatgccaaatagcaacatcaggcagacaatacccgaaattgcgaagaaaactgtctggtagcctgcgtggtcaaagagtatcccagtcggcgttgaaagcagcacaatcccaagcgaactggcaatttgaaaaccaatcagaaagatcgtcgacgacaggcgcttatcaaagtttgccacgctgtatttgaagacggatatgacacaaagtggaacctcaatggcatgtaacaacttcactaatgaaataatccaggggttaacgaacagcgcgcaggaaaggatacgcaacgccataatcacaactccgataagtaatgcattttttggccctacccgattcacaaagaaaggaataatcgccatgcacagcgcttcgagtaccacctggaatgagttgagataaccatacaggcgcgttcctacatcgtgtgattcgaataaacctgaataaaagacaggaaaaagttgttgatcaaaaatgttatagaaagaccacgtccccacaataaatatgacgaaaacccagaagtttcgatccttgaaaactgcgataaaatcctctttttttacccctcccgcatctgccgctacgcactggtgatccttatctttaaaacgcatgttgatcatcataaatacagcgccaaatagcgagaccaaccagaagttgatatggggactgatactaaaaaatatgccggcaaagaacgcgccaatagcatagccaaaagatccccaggcgcgcgctgttccatattcgaaatgaaaatttcgcgccattttttcggtgaagctatcaagcaaaccgcatcccgccagataccccaagccaaaaaatagcgcccccagaattagacctacagaaaaattgctttgcagtaacggttcataaacgtaaatcataaacggtccggtcaagaccaggatgaaactcatacaccagatgagcggtttcttcagaccgagtttatcctgaacgatgccgtagaacatcataaatagaatgctggtaaactggttgaccgaataaagtgtacctaattccgtccctgtcaaccctagatgtcctttcagccaaatagcgtataacgaccaccacagcgaccaggaaataaaaaagagaaatgagtaactggatgcaaaacgatagtacgcatttctgaatggaatattcagtgccataattacctgcctgtcgttaaaaaattcacgtcctatttagagataagagcgacttcgccgtttacttctcactattccagttcttgtcgacatggcagcgctgtcattgcccctttcgccgttactgcaagcgctccgcaacgttgagcgagatcgataattcgtcgcatttctctctcatctgtagataatcccgtagaggacagacctgtgagtaacccggcaacgaacgcatctcccgcccccgtgctatcgacacaattcacagacattccagcaaaatggtgaacttgtcctcgataacagaccaccaccccttctgcacctttagtcaccaacagcatggcgatctcatactcttttgccagggcgcatatatcctgatcgttctgtgtttttccactgataagtcgccattcttcttccgagagcttgacgacatccgccagttgtagcgcctgccgcaaacacaagcggagcaaatgctcgtcttgccatagatcttcacgaatattaggatcgaagctgacaaaacctccggcatgccggatcgccgtcatcgcagtaaatgcgctggtacgcgaaggctcggcagacaacgcaattgaacagagatgtaaccattcgccatgtcgccagcagggcaagtctgtcgtctctaaaaaaagatcggcactggggcggaccataaacgtaaatgaacgttccccttgatcgttcagatcgacaagcaccgtggatgtccggtgccattcatcttgcttcagatacgtgatatcgactccctcagttagcagcgttctttgcattaacgcaccaaaaggatcatcccccacccgacctataaacccacttgttccgcctaatctggcgattcccaccgcaacgttagctggcgcgccgccaggacaaggcagtaggcgcccgtctgattctggcaagagatctacgaccgcatcccctaaaacccatactttggctgacatttttttcccttaaattcatctgagttacgcatagtgataaacctctttttcgcaaaatcgtcatggatttactaaaacatgcatattcgatcacaaaacgtcatagttaacgttaacatttgtgatattcatcgcatttatgaaagtaagggactttatttttataaaagttaacgttaacaattcaccaaatttgcttaaccaggatgattaaaatgacgcaatctcgattgcatgcggcgcaaaacgccctagcaaaacttcatgagcaccggggtaacactttctatccccattttcacctcgcgcctcctgccgggtggatgaacgatccaaacggcctgatctggtttaacgatcgttatcacgcgttttatcaacatcatccgatgagcgaacactgggggccaatgcactggggacatgccaccagcgacgatatgatccactggcagcatgagcctattgcgctagcgccaggagacgataatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggaatcatgcacttccgcgatcctaaagtgtggcgtgaagccgacacatggtggatggtagtcggggcgaaagatccaggcaacacggggcagatcctgctttatcgcggcagttcgttgcgtgaatggaccttcgatcgcgtactggcccacgctgatgcgggtgaaagctatatgtgggaatgtccggactttttcagccttggcgatcagcattatctgatgttttccccgcagggaatgaatgccgagggatacagttaccgaaatcgctttcaaagtggcgtaatacccggaatgtggtcgccaggacgactttttgcacaatccgggcattttactgaacttgataacgggcatgacttttatgcaccacaaagctttttagcgaaggatggtcggcgtattgttatcggctggatggatatgtgggaatcgccaatgccctcaaaacgtgaaggatgggcaggctgcatgacgctggcgcgcgagctatcagagagcaatggcaaacttctacaacgcccggtacacgaagctgagtcgttacgccagcagcatcaatctgtctctccccgcacaatcagcaataaatatgttttgcaggaaaacgcgcaagcagttgagattcagttgcagtgggcgctgaagaacagtgatgccgaacattacggattacagctcggcactggaatgcggctgtatattgataaccaatctgagcgacttgttttgtggcggtattacccacacgagaatttagacggctaccgtagtattcccctcccgcagcgtgacacgctcgccctaaggatatttatcgatacatcatccgtggaagtatttattaacgacggggaagcggtgatgagtagtcgaatctatccgcagccagaagaacgggaactgtcgctttatgcctcccacggagtggctgtgctgcaacatggagcactctggctactgggttaacataatatcaggtggaacaacggatcaacagcgggcaagggatccgcgtcactcttcccccttcacgaccttcaataatatgcaatgcagcttcccgcccgataatgtcatgtggaagctgaattgtggtcagcggcggtaaaaacagatgcccgacgccaaccagattatcaaagcccattacggcgacatcctgcgggattcgtacccccttcgccagaagaacctgataagccacaaaggctgcgcgatcgttaccacatatcagaacatcaaaatctggtttgcccggtttgaagtgggcattgagtaaacttgcgagatcggtgtagtgatcatcacctgttgccatgtgaaattgtttcacctcagccagatctcgtccagcatcacgccaggcctgctcaaatccctgccgacgataccctgttgccaacgcactttccggtagccagaagcataacggttgacgatagcccgccgcgagcaaatgctgtgttgattcatattgtgcagtgtaatcatcagggatataactgggtaacgctgggtcatccgccacacagttcgccaatacaatattttcaccatacagagactcaggcagcgtgatatgtcgcagccccattgtagtatagataatgccatccggacggtgggcaagcagctgacgtgccgcgcgggcagcgtcatcttcagaaaaaatattgattaaaaaactattccagccgaactcgctggcggtttgctcaatggcaagcagaatatcaacagagaaaggagtggtagccgtgtcctgcgccagcacggcgagagtcgacggcttacgtccttgagcgcgcatcttaacgccgaagctgttttggcggatgagagaagattttcagcctgatacaga

(19) United States
(12) Patent Application Publication
(10) Pub. No.: US 2025/0361531 A1
(43) Pub. Date: Nov. 27, 2025

(54) MICROORGANISMS AND METHODS FOR PRODUCTION OF 4-HYDROXYBUTYRATE, 1,4-BUTANEDIOL AND RELATED COMPOUNDS

(71) Applicant: Genomatica, Inc., San Diego, CA (US)

(72) Inventors: Priti PHARKYA, San Diego, CA (US); Anthony P. BURGARD, Elizabeth, PA (US); Stephen J. VAN DIEN, Encinitas, CA (US); Robin E. OSTERHOUT, San Diego, CA (US); Mark J. BURK, San Diego, CA (US); John D. TRAWICK, La Mesa, CA (US); Michael P. KUCHINSKAS, Franklin, MA (US); Brian STEER, San Diego, CA (US); Stefan ANDRAE, San Diego, CA (US); Amit SHAH, San Diego, CA (US)

(21) Appl. No.: 19/187,905

(22) Filed: Apr. 23, 2025

Related U.S. Application Data
(62) Division of application No. 15/191,421, filed on Jun. 23, 2016, now Pat. No. 12,312,627.
Application No. 15/191,421 is a continuation of application No. 14/262,461, filed on Apr. 25, 2014, now abandoned.
(60) Provisional application No. 61/854,611, filed on Apr. 26, 2013.

(51) Int. Cl. C12P 7/18; C07C 31/20; C07C 47/19; C07C 211/09; C07D 307/33; C12N 1/20; C12N 9/04; C12N 9/10; C12N 15/52; C12P 7/24; C12P 7/42; C12P 7/62; C12P 13/00; C12P 17/04; C12P 19/32

(52) U.S. Cl. CPC C12P 7/18; C07C 31/207; C07C 47/19; C07C 211/09; C07D 307/33; C12N 1/20; C12N 9/0006; C12N 9/13; C12N 15/52; C12P 7/24; C12P 7/42; C12P 7/62; C12P 13/001; C12P 13/005; C12P 17/04; C12P 19/32; C12Y 208/03

(57) ABSTRACT

The invention provides non-naturally occurring microbial organisms having a 4-hydroxybutyrate, gamma-butyrolactone, 1,4-butanediol, 4-hydroxybutanal, 4-hydroxybutyryl-CoA and/or putrescine pathway and being capable of producing 4-hydroxybutyrate, wherein the microbial organism comprises one or more genetic modifications. The invention additionally provides methods of producing 4-hydroxybutyrate, gamma-butyrolactone, 1,4-butanediol, 4-hydroxybutanal, 4-hydroxybutyryl-CoA and/or putrescine or related products using the microbial organisms.

### BACKGROUND OF THE INVENTION

This application is a continuation of U.S. application Ser. No. 15/191,421, filed Jun. 23, 2016, which is a continuation of U.S. application Ser. No. 14/262,461, filed Apr. 25, 2014, which claims the benefit of priority of U.S. Provisional application Ser. No. 61/854,611, filed Apr. 26, 2013, the entire contents of which are incorporated herein by reference.

The instant application contains a Sequence Listing, which has been submitted via Patent Center. The Sequence Listing titled 199683-144003_US_SL.xml, which was created on Apr. 23, 2025 and is 782,431 bytes in size, is hereby incorporated by reference in its entirety.

This invention relates generally to in silico design of organisms and engineering of organisms, more particularly to organisms having 1,4-butanediol, 4-hydroxybutyryl-CoA, 4-hydroxybutanal or putrescine biosynthesis capability.

The compound 4-hydroxybutanoic acid (4-hydroxybutanoate, 4-hydroxybutyrate, 4-HB) is a 4-carbon carboxylic acid that has industrial potential as a building block for various commodity and specialty chemicals. In particular, 4-HB has the potential to serve as a new entry point into the 1,4-butanediol family of chemicals, which includes solvents, resins, polymer precursors, and specialty chemicals. 1,4-Butanediol (BDO) is a polymer intermediate and industrial solvent with a global market of about 3 billion lb/year. BDO is currently produced from petrochemical precursors, primarily acetylene, maleic anhydride, and propylene oxide.

For example, acetylene is reacted with 2 molecules of formaldehyde in the Reppe synthesis reaction (Kroschwitz and Grant, Encyclopedia of Chem. Tech, John Wiley and Sons, Inc., New York (1999)), followed by catalytic hydrogenation to form 1,4-butanediol. It has been estimated that 90% of the acetylene produced in the U.S. is consumed for butanediol production. Alternatively, it can be formed by esterification and catalytic hydrogenation of maleic anhydride, which is derived from butane. Downstream, butanediol can be further transformed; for example, by oxidation to γ-butyrolactone, which can be further converted to pyrrolidone and N-methyl-pyrrolidone, or hydrogenolysis to tetrahydrofuran. These compounds have varied uses as polymer intermediates, solvents, and additives, and have a combined market of nearly 2 billion lb/year. It is desirable to develop a method for production of these chemicals by alternative means that not only substitute renewable for petroleum-based feedstocks, and also use less energy- and capital-intensive processes.

Thus, there exists a need for alternative means for effectively producing commercial quantities of 1,4-butanediol and its chemical precursors. The present invention satisfies this need and provides related advantages as well.

### SUMMARY OF INVENTION

The invention provides non-naturally occurring microbial organisms having a 4-hydroxybutyrate, 1,4-butanediol, or other product pathway and being capable of producing 4-hydroxybutyrate, 1,4-butanediol, or other product wherein the microbial organism comprises one or more genetic modifications. The invention additionally provides methods of producing 4-hydroxybutyrate, 1,4-butanediol, or other product or related products using the microbial organisms.

The present invention also provides polypeptides and encoding isolated nucleic acid molecules. The polypeptides and nucleic acid molecules can be used in the production of 4-HB, GBL, 4-HBal, 4-HBCoA, BDO and/or putrescine. The invention also provides vectors containing the nucleic acid molecules, host cells containing the vectors and/or expressing the polypeptides of the invention, culture medium containing the host cells of the invention, methods of constructing the host cells of the invention, compositions having the polypeptides of the invention and at least one substrate for the polypeptide upon which to react, and methods of producing 4-HB, GBL, 4-HBal, 4-HBCoA, BDO and/or putrescine using a host cell of the invention.

### BRIEF DESCRIPTION OF THE DRAWINGS

FIG. 1 is a schematic diagram showing biochemical pathways to 4-hydroxybutyrate (4-HB) and to 1,4-butanediol production. The first 5 steps are endogenous to E. coli, while the remainder can be expressed heterologously. Enzymes catalyzing the biosynthetic reactions are: (1) succinyl-CoA synthetase; (2) CoA-independent succinic semialdehyde dehydrogenase; (3) α-ketoglutarate dehydrogenase; (4) glutamate:succinate semialdehyde transaminase; (5) glutamate decarboxylase; (6) CoA-dependent succinic semialdehyde dehydrogenase; (7) 4-hydroxybutanoate dehydrogenase; (8) α-ketoglutarate decarboxylase; (9) 4-hydroxybutyryl CoA:acetyl-CoA transferase; (10) butyrate kinase; (11) phosphotransbutyrylase; (12) aldehyde dehydrogenase; (13) alcohol dehydrogenase.

FIG. 2 is a schematic diagram showing homoserine biosynthesis in E. coli.

FIGS. 3A-3C show the production of 4-HB in glucose minimal medium using E. coli strains harboring plasmids expressing various combinations of 4-HB pathway genes. (FIG. 3A) 4-HB concentration in culture broth; (FIG. 3B) succinate concentration in culture broth; (FIG. 3C) culture OD, measured at 600 nm. Clusters of bars represent the 24 hour, 48 hour, and 72 hour (if measured) timepoints. The codes along the x-axis indicate the strain/plasmid combination used. The first index refers to the host strain: 1, MG1655 lacI^{Q}; 2, MG1655 ΔgabD lac^{Q}; 3, MG1655 ΔgabD ΔaldA lacI^{Q}. The second index refers to the plasmid combination used: 1, pZE13-0004-0035 and pZA33-0036; 2, pZE13-0004-0035 and pZA33-0010n; 3, pZE13-0004-0008 and pZA33-0036; 4, pZE13-0004-0008 and pZA33-0010n; 5, Control vectors pZE13 and pZA33.

FIG. 4 shows the production of 4-HB from glucose in E. coli strains expressing α-ketoglutarate decarboxylase from Mycobacterium tuberculosis. Strains 1-3 contain pZE13-0032 and pZA33-0036. Strain 4 expresses only the empty vectors pZE13 and pZA33. Host strains are as follows: 1 and 4, MG1655 lacI^{Q}; 2, MG1655 ΔgabD lacI^{Q}; 3, MG1655 ΔgabD ΔaldA lacI^{Q}. The bars refer to concentration at 24 and 48 hours.

FIG. 5 shows the production of BDO from 10 mM 4-HB in recombinant E. coli strains. Numbered positions correspond to experiments with MG1655 lacI^{Q} containing pZA33-0024, expressing cat2 from P. gingivalis, and the following genes expressed on pZE13: 1, none (control); 2, 0002; 3, 0003; 4, 0003n; 5, 0011; 6, 0013; 7, 0023; 8, 0025; 9, 0008n; 10, 0035. Gene numbers are defined in Table 6. For each position, the bars refer to aerobic, microaerobic, and anaerobic conditions, respectively. Microaerobic conditions were created by sealing the culture tubes but not evacuating them.

FIGS. 6A-6H show the mass spectrum of 4-HB and BDO produced by MG1655 lacI^{Q} pZE13-0004-0035-0002 pZA33-0034-0036 grown in M9 minimal medium supplemented with 4 g/L unlabeled glucose (FIGS. 6A, 6C, 6E and 6G) uniformly labeled ¹³C-glucose (FIGS. 6B, 6D, 6F and 6H). FIGS. 6A and 6B, mass 116 characteristic fragment of derivatized BDO, containing 2 carbon atoms; FIGS. 6C and 6D, mass 177 characteristic fragment of derivatized BDO, containing 1 carbon atom; FIGS. 6E and 6F, mass 117 characteristic fragment of derivatized 4-HB, containing 2 carbon atoms; FIGS. 6G and 6H, mass 233 characteristic fragment of derivatized 4-HB, containing 4 carbon atoms.

FIG. 7 is a schematic process flow diagram of bioprocesses for the production of γ-butyrolactone. Upper panel illustrates fed-batch fermentation with batch separation and lower panel illustrates fed-batch fermentation with continuous separation.

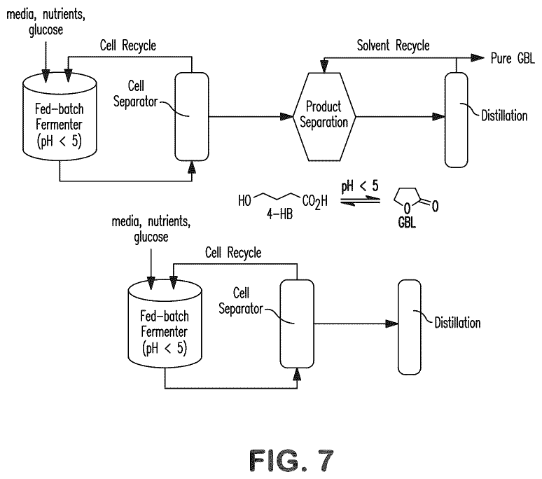

FIGS. 8A and 8B show exemplary 1,4-butanediol (BDO) pathways. FIG. 8A shows BDO pathways from succinyl-CoA. FIG. 8B shows BDO pathways from alpha-ketoglutarate.

FIGS. 9A-9C show exemplary BDO pathways. FIGS. 9A and 9B show pathways from 4-aminobutyrate. FIG. 9C shows a pathway from acetoactyl-CoA to 4-aminobutyrate.

FIG. 10 shows exemplary BDO pathways from alpha-ketoglutarate.

FIG. 11 shows exemplary BDO pathways from glutamate.

FIG. 12 shows exemplary BDO pathways from acetoacetyl-CoA.

FIG. 13 shows exemplary BDO pathways from homoserine.

FIGS. 14A-14C show the nucleotide and amino acid sequences of E. coli succinyl-CoA synthetase. FIG. 14A shows the nucleotide sequence (SEQ ID NO:46) of the E. coli sucCD operon. FIGS. 14B (SEQ ID NO:47) and 14C (SEQ ID NO:48) show the amino acid sequences of the succinyl-CoA synthetase subunits encoded by the sucCD operon.

FIGS. 15A and 15B show the nucleotide and amino acid sequences of Mycobacterium bovis alpha-ketoglutarate decarboxylase. FIG. 15A shows the nucleotide sequence (SEQ ID NO:49) of Mycobacterium bovis sucA gene. FIG. 15B shows the amino acid sequence (SEQ ID NO:50) of M. bovis alpha-ketoglutarate decarboxylase.

FIG. 16 shows biosynthesis in E. coli of 4-hydroxybutyrate from glucose in minimal medium via alpha-ketoglutarate under anaerobic (microaerobic) conditions. The host strain is ECKh-401. The experiments are labeled based on the upstream pathway genes present on the plasmid pZA33 as follows: 1) 4hbd-sucA; 2) sucCD-sucD-4hbd, 3) sucCD-sucD-4hbd-sucA.

FIG. 17 shows biosynthesis in E. coli of 4-hydroxybutyrate from glucose in minimal medium via succinate and alpha-ketoglutarate. The host strain is wild-type MG1655. The experiments are labeled based on the genes present on the plasmids pZE13 and pZA33 as follows: 1) empty control vectors 2) empty pZE13, pZA33-4hbd; 3) pZE13-sucA, pZA33-4hbd.

FIG. 18 A shows the nucleotide sequence (SEQ ID NO:51) of CoA-dependent succinate semialdehyde dehydrogenase (sucD) from Porphyromonas gingivalis, and FIG. 18B shows the encoded amino acid sequence (SEQ ID NO:52).

FIG. 19A shows the nucleotide sequence (SEQ ID NO:53) of 4-hydroxybutyrate dehydrogenase (4hbd) from Porphyromonas gingivalis, and FIG. 19B shows the encoded amino acid sequence (SEQ ID NO:54).

FIG. 20A shows the nucleotide sequence (SEQ ID NO:55) of 4-hydroxybutyrate CoA transferase (cat2) from Porphyromonas gingivalis, and FIG. 20B shows the encoded amino acid sequence (SEQ ID NO:56).

FIG. 21A shows the nucleotide sequence (SEQ ID NO:57) of phosphotransbutyrylase (ptb) from Clostridium acetobutylicum, and FIG. 21B shows the encoded amino acid sequence (SEQ ID NO:58).

FIG. 22A shows the nucleotide sequence (SEQ ID NO:59) of butyrate kinase (buk1) from Clostridium acetobutylicum, and FIG. 22B shows the encoded amino acid sequence (SEQ ID NO:60).

FIGS. 23A-23D show alternative nucleotide sequences for C. acetobutylicum 020 (phosphtransbutryrlase) with altered codons for more prevalent E. coli codons relative to the C. acetobutylicum native sequence. FIGS. 23A-23D (020A-020D, SEQ ID NOS:61-64, respectively) contain sequences with increasing numbers of rare E. coli codons replaced by more prevalent codons (A<B<C<D).

FIGS. 24A-24D show alternative nucleotide sequences for C. acetobutylicum 021 (butyrate kinase) with altered codons for more prevalent E. coli codons relative to the C. acetobutylicum native sequence. FIGS. 24A-24D (021A-021B, SEQ ID NOS:65-68, respectively) contain sequences with increasing numbers of rare E. coli codons replaced by more prevalent codons (A<B<C<D).

FIGS. 25A and 25B show improved expression of butyrate kinase (BK) and phosphotransbutyrylase (PTB) with optimized codons for expression in E. coli. FIG. 25A shows sodium dodecyl sulfate polyacrylamide gel electrophoresis (SDS-PAGE) stained for proteins with Coomassie blue; lane 1, control vector with no insert; lane 2, expression of C. acetobutylicum native sequences in E. coli; lane 3, expression of 020B-021B codon optimized PTB-BK; lane 4, expression of 020C-021C codon optimized PTB-BK. The positions of BK and PTB are shown. FIG. 25B shows the BK and PTB activities of native C. acetobutylicum sequence (2021n) compared to codon optimized 020B-021B (2021B) and 020C-021C (2021C).

FIG. 26 shows production of BDO and gamma-butyrolactone (GBL) in various strains expressing BDO producing enzymes: Cat2 (034); 2021n; 2021B; 2021C.

FIG. 27A shows the nucleotide sequence (SEQ ID NO:69) of the native Clostridium beijerinckii ald gene (025n), and FIG. 27B shows the encoded amino acid sequence (SEQ ID NO:70).

FIGS. 28A-28D show alternative gene sequences for the Clostridium beijerinckii ald gene (025A-025D, SEQ ID NOs:71-74, respectively), in which increasing numbers of rare codons are replaced by more prevalent codons (A<B<C<D).

FIG. 29 shows expression of native C. beijerinckii ald gene and codon optimized variants; no ins (control with no insert), 025n, 025A, 025B, 025C, 025D.

FIGS. 30A-30B show BDO or BDO and ethanol production in various strains. FIG. 30A shows BDO production in strains containing the native C. beijerinckii ald gene (025n) or variants with optimized codons for expression in E. coli (025A-025D). FIG. 30B shows production of ethanol and BDO in strains expressing the C. acetobutylicum AdhE2 enzyme (002C) compared to the codon optimized variant 025B. The third set shows expression of P. gingivalis sucD (035). In all cases, P. gingivalis Cat2 (034) is also expressed.

FIG. 31A shows the nucleotide sequence (SEQ ID NO:75) of the adh1 gene from Geobacillus thermoglucosidasius, and FIG. 31B shows the encoded amino acid sequence (SEQ ID NO:76).

FIG. 32A shows the expression of the Geobacillus thermoglucosidasius adh1 gene in E. coli. Either whole cell lysates or supernatants were analyzed by SDS-PAGE and stained with Coomassie blue for plasmid with no insert, plasmid with 083 (Geotrichum capitatum N-benzyl-3-pyrrolidinol dehydrogenase) and plasmid with 084 (Geobacillus thermoglucosidasius adh1) inserts. FIG. 32B shows the activity of 084 with butyraldehyde (diamonds) or 4-hydroxybutyraldehyde (squares) as substrates.

FIG. 33 shows the production of BDO in various strains: plasmid with no insert; 025B, 025B-026n; 025B-026A; 025B-026B; 025B-026C; 025B-050; 025B-052; 025B-053; 025B-055; 025B-057; 025B-058; 025B-071; 025B-083; 025B-084; PTSlacO-025B; PTSlacO-025B-026n.

FIG. 34 shows a plasmid map for the vector pRE118-V2.

FIG. 35 shows the sequence (SEQ ID NO:77) of the ECKh-138 region encompassing the aceF and lpdA genes. The K. pneumonia lpdA gene is underlined, and the codon changed in the Glu354Lys mutant shaded.

FIG. 36 shows the protein sequence comparison of the native E. coli lpdA (SEQ ID NO: 78) and the mutant K. pneumonia lpdA (SEQ ID NO:79).

FIG. 37 shows 4-hydroxybutyrate (left bars) and BDO (right bars) production in the strains AB3, MG1655 ΔldhA and ECKh-138. All strains expressed E. coli sucCD, P. gingivalis sucD, P. gingivalis 4hbd on the medium copy plasmid pZA33, and P. gingivalis Cat2, C. acetobutylicum AdhE2 on the high copy plasmid pZE13.

FIG. 38 shows the nucleotide sequence (SEQ ID NO:80) of the 5′ end of the aceE gene fused to the pflB-p6 promoter and ribosome binding site (RBS). The 5′ italicized sequence shows the start of the aroP gene, which is transcribed in the opposite direction from the pdh operon. The 3′ italicized sequence shows the start of the aceE gene. In upper case: pflB RBS. Underlined: FNR binding site. In bold: pflB-p6 promoter sequence.

FIG. 39 shows the nucleotide sequence (SEQ ID NO:81) in the aceF-lpdA region in the strain ECKh-456.

FIG. 40 shows the production of 4-hydroxybutyrate, BDO and pyruvate (left to right bars, respectively) for each of strains ECKh-439, ECKh-455 and ECKh-456.

FIG. 41A shows a schematic of the recombination sites for deletion of the mdh gene. FIG. 41B shows the nucleotide sequence of the PCR product (SEQ ID NO:82) and the encoded amino acid sequence (SEQ ID NO:83) of the amplification of chloramphenicol resistance gene (CAT) flanked by FRT sites and homology regions from the mdh gene from the plasmid pKD3.

FIG. 42 shows the sequence (SEQ ID NO:84) of the arcA deleted region in strain ECKh-401.

FIG. 43 shows the sequence (SEQ ID NO:85) of the region encompassing a mutated gltA gene of strain ECKh-422.

FIGS. 44A-44B show the citrate synthase activity of wild type gltA gene product (FIG. 44A) and the R163L mutant (FIG. 44B). The assay was performed in the absence (diamonds) or presence of 0.4 mM NADH (squares).

FIG. 45 shows the 4-hydroxybutyrate (left bars) and BDO (right bars) production in strains ECKh-401 and ECKh-422, both expressing genes for the complete BDO pathway on plasmids.

FIG. 46 shows central metabolic fluxes and associated 95% confidence intervals from metabolic labeling experiments. Values are molar fluxes normalized to a glucose uptake rate of 1 mmol/hr. The result indicates that carbon flux is routed through citrate synthase in the oxidative direction and that most of the carbon enters the BDO pathway rather than completing the TCA cycle.

FIGS. 47A-47B show extracellular product formation for strains ECKh-138 (FIG. 47A) and ECKh-422 (FIG. 47B), both expressing the entire BDO pathway on plasmids. The products measured were acetate (Ace), pyruvate (Pyr), 4-hydroxybutyrate (4HB), 1,4-butanediol (BDO), ethanol (EtOH), and other products, which include gamma-butyrolactone (GBL), succinate, and lactate.

FIG. 48 shows the sequence (SEQ ID NO:86) of the region following replacement of PEP carboxylase (ppc) by H. influenzae phosphoenolpyruvate carboxykinase (pepck). The pepck coding region is underlined.

FIG. 49 shows growth of evolved pepCK strains grown in minimal medium containing 50 mM NaHCO₃.

FIG. 50 shows product formation in strain ECKh-453 expressing P. gingivalis Cat2 and C. beijerinckii Ald on the plasmid pZS*13. The products measured were 1,4-butanediol (BDO), pyruvate, 4-hydroxybutyrate (4HB), acetate, γ-butyrolactone (GBL) and ethanol.

FIG. 51 shows BDO production of two strains, ECKh-453 and ECKh-432. Both contain the plasmid pZS*13 expressing P. gingivalis Cat2 and C. beijerinckii Ald. The cultures were grown under microaerobic conditions, with the vessels punctured with 27 or 18 gauge needles, as indicated.

FIG. 52 shows the nucleotide sequence (SEQ ID NO:87) of the genomic DNA of strain ECKh-426 in the region of insertion of a polycistronic DNA fragment containing a promoter, sucCD gene, sucD gene, 4hbd gene and a terminator sequence.

FIG. 53 shows the nucleotide sequence (SEQ ID NO:88) of the chromosomal region of strain ECKh-432 in the region of insertion of a polycistronic sequence containing a promoter, sucA gene, C. kluyveri 4hbd gene and a terminator sequence.

FIG. 54 shows BDO synthesis from glucose in minimal medium in the ECKh-432 strain having upstream BDO pathway encoding genes intergrated into the chromosome and containing a plasmid harboring downstream BDO pathway genes.

FIG. 55 shows a PCR product (SEQ ID NO:89, top strand, and SEQ ID NO:519, bottom strand) containing the non-phosphotransferase (non-PTS) sucrose utilization genes flanked by regions of homology to the rrnC region.

FIG. 56 shows a schematic diagram of the integrations site in the rrnC operon.

FIG. 57 shows average product concentration, normalized to culture OD600, after 48 hours of growth of strain ECKh-432 grown on glucose and strain ECKh-463 grown on sucrose. Both contain the plasmid pZS*13 expressing P. gingivalis Cat2 and C. beijerinckii Ald. The data is for 6 replicate cultures of each strain. The products measured were 1,4-butanediol (BDO), 4-hydroxybutyrate (4HB), γ-butyrolactone (GBL), pyruvate (PYR) and acetate (ACE)(left to right bars, respectively).

FIG. 58 shows exemplary pathways to 1,4-butanediol from succinyl-CoA and alpha-ketoglutarate. Abbreviations: A) Succinyl-CoA reductase (aldehyde forming), B) Alpha-ketoglutarate decarboxylase, C) 4-Hydroxybutyrate dehydrogenase, D) 4-Hydroxybutyrate reductase, E) 1,4-Butanediol dehydrogenase.

FIG. 59A shows the nucleotide sequence (SEQ ID NO:90) of carboxylic acid reductase from Nocardia iowensis (GNM_720), and FIG. 59B shows the encoded amino acid sequence (SEQ ID NO:91).

FIG. 60A shows the nucleotide sequence (SEQ ID NO:92) of phosphopantetheine transferase, which was codon optimized, and FIG. 60B shows the encoded amino acid sequence (SEQ ID NO:93).

FIG. 61 shows a plasmid map of plasmid pZS*-13S-720 72lopt.

FIGS. 62A and 62B show pathways to 1,4-butanediol from succinate, succcinyl-CoA, and alpha-ketoglutarate. Abbreviations: A) Succinyl-CoA reductase (aldehyde forming), B) Alpha-ketoglutarate decarboxylase, C) 4-Hydroxybutyrate dehydrogenase, D) 4-Hydroxybutyrate reductase, E) 1,4-Butanediol dehydrogenase, F) Succinate reductase, G) Succinyl-CoA transferase, H) Succinyl-CoA hydrolase, I) Succinyl-CoA synthetase (or Succinyl-CoA ligase), J) Glutamate dehydrogenase, K) Glutamate transaminase, L) Glutamate decarboxylase, M) 4-aminobutyrate dehydrogenase, N) 4-aminobutyrate transaminase, O) 4-Hydroxybutyrate kinase, P) Phosphotrans-4-hydroxybutyrylase, Q) 4-Hydroxybutyryl-CoA reductase (aldehyde forming), R) 4-hydroxybutyryl-phosphate reductase, S) Succinyl-CoA reductase (alcohol forming), T) 4-Hydroxybutyryl-CoA transferase, U) 4-Hydroxybutyryl-CoA hydrolase, V) 4-Hydroxybutyryl-CoA synthetase (or 4-Hydroxybutyryl-CoA ligase), W) 4-Hydroxybutyryl-CoA reductase (alcohol forming), X) Alpha-ketoglutarate reductase, Y) 5-Hydroxy-2-oxopentanoate dehydrogenase, Z) 5-Hydroxy-2-oxopentanoate decarboxylase, AA) 5-hydroxy-2-oxopentanoate dehydrogenase (decarboxylation).

FIG. 63 shows pathways to putrescine from succinate, succcinyl-CoA, and alpha-ketoglutarate. Abbreviations: A) Succinyl-CoA reductase (aldehyde forming), B) Alpha-ketoglutarate decarboxylase, C) 4-Aminobutyrate reductase, D) Putrescine dehydrogenase, E) Putrescine transaminase, F) Succinate reductase, G) Succinyl-CoA transferase, H) Succinyl-CoA hydrolase, I) Succinyl-CoA synthetase (or Succinyl-CoA ligase), J) Glutamate dehydrogenase, K) Glutamate transaminase, L) Glutamate decarboxylase, M) 4-Aminobutyrate dehydrogenase, N) 4-Aminobutyrate transaminase, O) Alpha-ketoglutarate reductase, P) 5-Amino-2-oxopentanoate dehydrogenase, Q) 5-Amino-2-oxopentanoate transaminase, R) 5-Amino-2-oxopentanoate decarboxylase, S) Omithine dehydrogenase, T) Ornithine transaminase, U) Ornithine decarboxylase.

FIG. 64A shows the nucleotide sequence (SEQ ID NO:94) of carboxylic acid reductase from Mycobacterium smegmatis mc(2)155 (designated 890), and FIG. 64B shows the encoded amino acid sequence (SEQ ID NO:95).

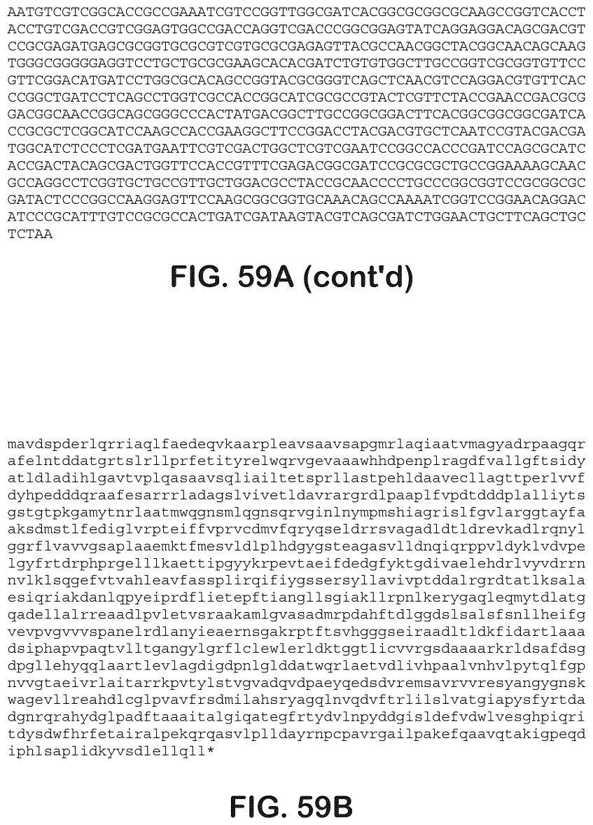

FIG. 65A shows the nucleotide sequence (SEQ ID NO:96) of carboxylic acid reductase from Mycobacterium avium subspecies paratuberculosis K-10 (designated 891), and FIG. 65B shows the encoded amino acid sequence (SEQ ID NO:97).

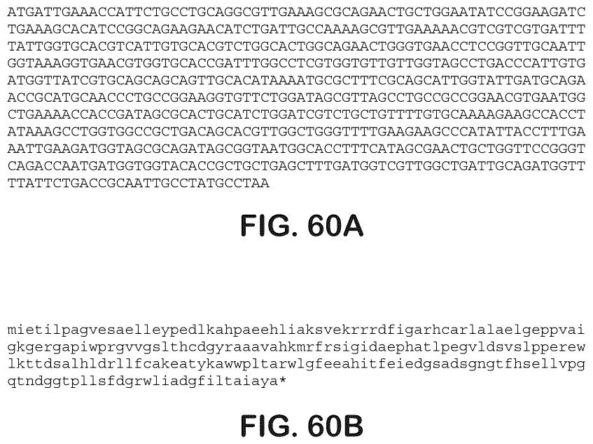

FIG. 66A shows the nucleotide sequence (SEQ ID NO:98) of carboxylic acid reductase from Mycobacterium marinum M (designated 892), and FIG. 66B shows the encoded amino acid sequence (SEQ ID NO:99).

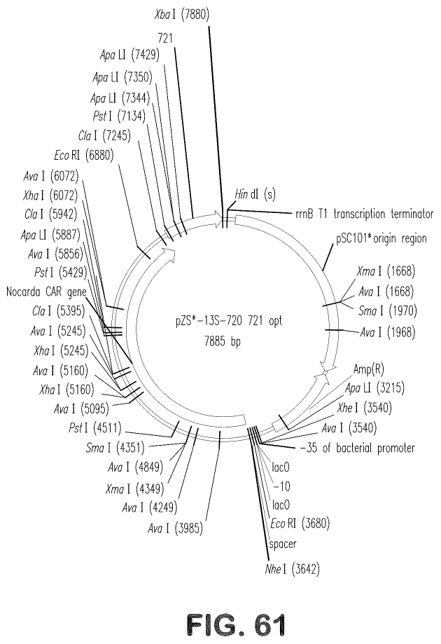

FIG. 67 shows a schematic of the E. coli MG1655 chromosome between nucleotides 760928 and 765930.

FIGS. 68A-68E show a schematic of the sequences and oligonucleotides used in the construction of the sucCD deletion.

FIG. 69 shows an exemplary BDO pathway. Enzyme names for each numbered step are as follows: (1) alpha-ketoglutarate decarboxylase; (2) CoA-dependent succinate semialdehyde dehydrogenase; (3) 4-hydroxybutyrate dehydrogenase; (4) 4-hydroxybutyryl-CoA transferase; (5) 4-hydroxybutyryl-CoA reductase; (6) 4-hydroxybutyraldehyde reductase.

FIG. 70 shows an exemplary pathway for the formation of BDO from glucose. Abbreviations: G6P-glucose-6-phosphate, PEP—phosphoenolpyruvate, PYR—pyruvate, OA—oxaloacetate, ACCOA—acetyl-CoA, CIT—citrate, ICI—isocitrate, AKG—alpha-ketoglutarate, SUCCOA—succinyl-CoA, SUCSAL—succinate semialdehyde, 4HB—4-hydroxybutyrate, 4HBCoA—4-hydroxybutyryl-CoA, 4HBALD—4hydroxybutyraldehyde, BDO—1,4-butanediol, GBL—gamma-butyrolactone. Genes of interest: ppc—PEP carboxylase, sucCD—succinyl-CoA synthetase, sucAB-lpdA—subunits of the AKG dehydrogenase complex, ybgC, tesB-acyl-CoenzymeA thioesterases

FIG. 71 shows the average BDO and 4HB numbers from four replicates of a 4HB producing host strain that had the gene ppc overexpressed compared with the corresponding averages from four replicates of the control strain.

FIG. 72 shows the reduction in the average ethanol numbers from the four replicates of the strain that had ppc overexpressed and compared with those from the control.

FIG. 73 shows the average BDO and 4HB numbers from four replicates of a 4HB producing host strain that had the genes sucAB and mutant lpdA overexpressed on pZS* compared with the corresponding averages from four replicates of the control strain. ALD and ADH were expressed on the plasmid pPZSX23R.

FIG. 74 shows the reduction in the average glutamate numbers from the four replicates of the strain that had the genes sucAB and mutant lpdA overexpressed on pZS* and compared with those from the control.

FIG. 75 shows apparent equilibrium constant for lactonization of 4-HB to GBL as a function of pH (at 22° C.)(from Efe et al., Biotechnol. Bioeng. 99:1392-1406 (2008)).

FIG. 76 shows the average BDO and 4HB numbers from four replicates of a host strain capable of producing 4-hydrobutyryl-CoA that had the esterase integrated (2387) compared with the corresponding averages from four replicates of the control strain (2237).

FIG. 77 shows the average GBL numbers from four replicates of a host strain that had the esterase integrated (2387) compared with the corresponding averages from four replicates of the control strain (2237). Lower GBL was observed with the strains that had the esterase integrated.

FIG. 78 shows the average BDO and 4HB numbers from four replicates of a host strain that had the genes sucCD deleted (4269) compared with the corresponding averages from four replicates of the control strain (4070).

FIG. 79 shows the average BDO and 4HB numbers from three replicates of a host strain that had the genes ybgC and tesB deleted (1197) compared with the corresponding averages from three replicates of the control strain (1136).

FIG. 80 shows the average GBL numbers from three replicates of a host strain that had the genes ybgC and tesB deleted (1197) compared with the corresponding averages from three replicates of the control strain (1136).

FIG. 81 shows the profile for the anion exchange elution, backflux activity, as well as the SDS page for the fractions.

FIG. 82 shows the profile from the size exclusion chromatography, the backflux activity, as well as the SDS page for the fractions.

FIG. 83 shows backflux activity from the size exclusion column and the SDS page.

FIG. 84 shows the yjgB gene was cloned with a streptavidin tag, expressed and purified in order to characterize its properties. The results of the purification are via SDS-PAGE.

FIG. 85 shows yjgB activity in 100 mM BDO.

FIG. 86 shows the average BDO, 4HB and GBL numbers for the strain 1872 versus strain 1889.

FIG. 87 shows the average BDO, 4HB and GBL numbers for the strain 956 versus strain 879.

FIG. 88 shows the average BDO, 4HB and GBL numbers of the strains, 1889, 2424, 2611 and 2471.

FIG. 89A shows the nucleotide sequence of the Yersinia gene (locus yinte0001_13710)(SEQ ID NO:177), and FIG. 89B shows the encoded amino acid sequence (SEQ ID NO:179).

FIG. 90A shows the nucleotide sequence of the gene from Agrobacterium tumefaciens (SEQ ID NO:178), and FIG. 90B shows the encoded amino acid sequence (SEQ ID NO:180).

### DETAILED DESCRIPTION OF THE INVENTION

The present invention is directed to the design and production of cells and organisms having biosynthetic production capabilities for 4-hydroxybutanoic acid (4-hydroxybutyrate, 4-HB), γ-butyrolactone, 1,4-butanediol (BDO), 4-hydroxybutanal (4-hydroxybutyraldehyde, 4-HBal), 4-hydroxybutyryl-CoA(4-HBCoA) and/or putrescine. The invention, in particular, relates to the design of microbial organisms capable of producing BDO, 4-HBal, 4-HBCoA and/or putrescine by introducing one or more nucleic acids encoding a BDO, 4-HBal, 4-HBCoA and/or putrescine pathway enzyme.

In one embodiment, the invention utilizes in silico stoichiometric models of Escherichia coli metabolism that identify metabolic designs for biosynthetic production of 4-hydroxybutanoic acid (4-HB), 1,4-butanediol (BDO), 4-HBal, 4-HBCoA and/or putrescine. The results described herein indicate that metabolic pathways can be designed and recombinantly engineered to achieve the biosynthesis of 4-HBal, 4-HBCoA or 4-HB and downstream products such as 1,4-butanediol or putrescine in Escherichia coli and other cells or organisms. Biosynthetic production of 4-HB, 4-HBal, 4-HBCoA, BDO and/or putrescine, for example, for the in silico designs can be confirmed by construction of strains having the designed metabolic genotype. These metabolically engineered cells or organisms also can be subjected to adaptive evolution to further augment 4-HB, 4-HBal, 4-HBCoA, BDO and/or putrescine biosynthesis, including under conditions approaching theoretical maximum growth.

In certain embodiments, the 4-HB, 4-HBal, 4-HBCoA, BDO and/or putrescine biosynthesis characteristics of the designed strains make them genetically stable and particularly useful in continuous bioprocesses. Separate strain design strategies were identified with incorporation of different non-native or heterologous reaction capabilities into E. coli or other host organisms leading to 4-HB and 1,4-butanediol producing metabolic pathways from either CoA-independent succinic semialdehyde dehydrogenase, succinyl-CoA synthetase and CoA-dependent succinic semialdehyde dehydrogenase, or glutamate:succinic semialdehyde transaminase. In silico metabolic designs were identified that resulted in the biosynthesis of 4-HB in both E. coli and yeast species from each of these metabolic pathways. The 1,4-butanediol intermediate γ-butyrolactone can be generated in culture by spontaneous cyclization under conditions at pH<7.5, particularly under acidic conditions, such as below pH 5.5, for example, pH<7, pH<6.5, pH<6, and particularly at pH<5.5 or lower.

Strains identified via the computational component of the platform can be put into actual production by genetically engineering any of the predicted metabolic alterations which lead to the biosynthetic production of 4-HB, 1,4-butanediol or other intermediate and/or downstream products. In yet a further embodiment, strains exhibiting biosynthetic production of these compounds can be further subjected to adaptive evolution to further augment product biosynthesis. The levels of product biosynthesis yield following adaptive evolution also can be predicted by the computational component of the system.

In other specific embodiments, microbial organisms were constructed to express a 4-HB biosynthetic pathway encoding the enzymatic steps from succinate to 4-HB and to 4-HB-CoA. Co-expression of succinate coenzyme A transferase, CoA-dependent succinic semialdehyde dehydrogenase, NAD-dependent 4-hydroxybutyrate dehydrogenase and 4-hydroxybutyrate coenzyme A transferase in a host microbial organism resulted in significant production of 4-HB compared to host microbial organisms lacking a 4-HB biosynthetic pathway. In a further specific embodiment, 4-HB-producing microbial organisms were generated that utilized α-ketoglutarate as a substrate by introducing nucleic acids encoding α-ketoglutarate decarboxylase and NAD-dependent 4-hydroxybutyrate dehydrogenase.

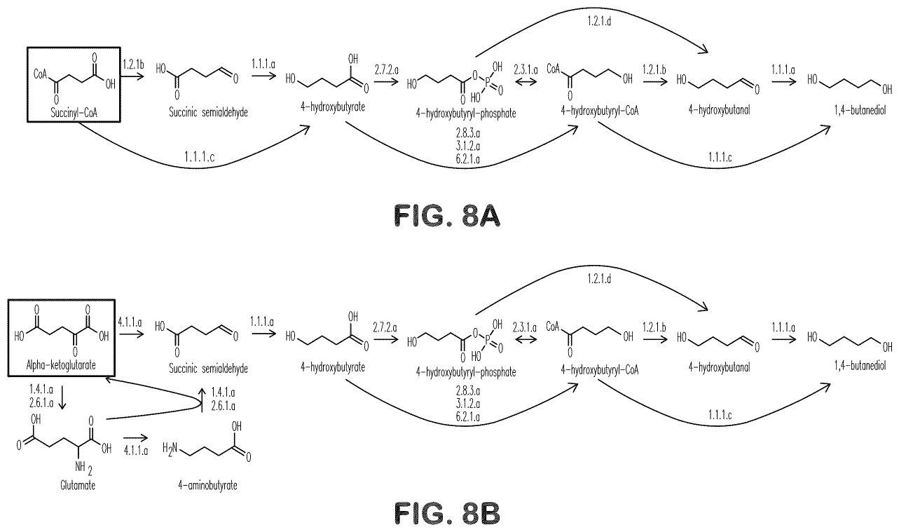

In another specific embodiment, microbial organisms containing a 1,4-butanediol (BDO) biosynthetic pathway were constructed that biosynthesized BDO when cultured in the presence of 4-HB. The BDO biosynthetic pathway consisted of a nucleic acid encoding either a multifunctional aldehyde/alcohol dehydrogenase or nucleic acids encoding an aldehyde dehydrogenase and an alcohol dehydrogenase. To support growth on 4-HB substrates, these BDO-producing microbial organisms also expressed 4-hydroxybutyrate CoA transferase or 4-butyrate kinase in conjunction with phosphotranshydroxybutyrlase. In yet a further specific embodiment, microbial organisms were generated that synthesized BDO through exogenous expression of nucleic acids encoding a functional 4-HB biosynthetic pathway and a functional BDO biosynthetic pathway. The 4-HB biosynthetic pathway consisted of succinate coenzyme A transferase, CoA-dependent succinic semialdehyde dehydrogenase, NAD-dependent 4-hydroxybutyrate dehydrogenase and 4-hydroxybutyrate coenzyme A transferase. The BDO pathway consisted of a multifunctional aldehyde/alcohol dehydrogenase. Further described herein are additional pathways for production of BDO (see FIGS. 8-13).

In a further embodiment, described herein is the cloning and expression of a carboxylic acid reductase enzyme that functions in a 4-hydroxybutanal, 4-hydroxybutyryl-CoA or 1,4-butanediol metabolic pathway. Advantages of employing a carboxylic acid reductase as opposed to an acyl-CoA reductase to form 4-hydroxybutyraldehyde (4-hydroxybutanal) include lower ethanol and GBL byproduct formation accompanying the production of BDO. Also disclosed herein is the application of carboxylic acid reductase as part of additional numerous pathways to produce 1,4-butanediol and putrescine from the tricarboxylic acid (TCA) cycle metabolites, for example, succinate, succinyl-CoA, and/or alpha-ketoglutarate.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a biosynthetic pathway for a 4-HB, 4-HBal, 4-HBCoA, BDO or putrescine family of compounds.

Ametabolic modification refers to a biochemical reaction that is altered from its naturally occurring state. Therefore, non-naturally occurring microorganisms can have genetic modifications to nucleic acids encoding metabolic polypeptides or, functional fragments thereof. Exemplary metabolic modifications are disclosed herein.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “4-hydroxybutanoic acid” is intended to mean a 4-hydroxy derivative of butyric acid having the chemical formula C₄H₈O₃ and a molecular mass of 104.11 g/mol (126.09 g/mol for its sodium salt). The chemical compound 4-hydroxybutanoic acid also is known in the art as 4-HB, 4-hydroxybutyrate, gamma-hydroxybutyric acid or GHB. The term as it is used herein is intended to include any of the compound's various salt forms and include, for example, 4-hydroxybutanoate and 4-hydroxybutyrate. Specific examples of salt forms for 4-HB include sodium 4-HB and potassium 4-HB. Therefore, the terms 4-hydroxybutanoic acid, 4-HB, 4-hydroxybutyrate, 4-hydroxybutanoate, gamma-hydroxybutyric acid and GHB as well as other art recognized names are used synonymously herein.

As used herein, the term “monomeric” when used in reference to 4-HB is intended to mean 4-HB in a non-polymeric or underivatized form. Specific examples of polymeric 4-HB include poly-4-hydroxybutanoic acid and copolymers of, for example, 4-HB and 3-HB. A specific example of a derivatized form of 4-HB is 4-HB-CoA. Other polymeric 4-HB forms and other derivatized forms of 4-HB also are known in the art.

As used herein, the term “γ-butyrolactone” is intended to mean a lactone having the chemical formula C₄H₆O₂ and a molecular mass of 86.089 g/mol. The chemical compound γ-butyrolactone also is know in the art as GBL, butyrolactone, 1,4-lactone, 4-butyrolactone, 4-hydroxybutyric acid lactone, and gamma-hydroxybutyric acid lactone. The term as it is used herein is intended to include any of the compound's various salt forms.

As used herein, the term “1,4-butanediol” is intended to mean an alcohol derivative of the alkane butane, carrying two hydroxyl groups which has the chemical formula C₄H₁₀O₂ and a molecular mass of 90.12 g/mol. The chemical compound 1,4-butanediol also is known in the art as BDO and is a chemical intermediate or precursor for a family of compounds referred to herein as BDO family of compounds.

As used herein, the term “4-hydroxybutanal” is intended to mean an aledehyde having the chemical formula C₄H₈O₂ and a molecular mass of 88.10512 g/mol. The chemical compound 4-hydroxybutanal (4-HBal) is also known in the art as 4-hydroxybutyraldehyde.

As used herein, the term “putrescine” is intended to mean a diamine having the chemical formula C₄H₁₂N₂ and a molecular mass of 88.15148 g/mol. The chemical compound putrescine is also known in the art as 1,4-butanediamine, 1,4-diaminobutane, butylenediamine, tetramethylenediamine, tetramethyldiamine, and 1,4-butylenediamine.

As used herein, the term “tetrahydrofuran” is intended to mean a heterocyclic organic compound corresponding to the fully hydrogenated analog of the aromatic compound furan which has the chemical formula C₄H₈O and a molecular mass of 72.11 g/mol. The chemical compound tetrahydrofuran also is known in the art as THF, tetrahydrofuran, 1,4-epoxybutane, butylene oxide, cyclotetramethylene oxide, oxacyclopentane, diethylene oxide, oxolane, furanidine, hydrofuran, tetra-methylene oxide. The term as it is used herein is intended to include any of the compound's various salt forms.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

The non-naturally occurring microbal organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

In the case of gene disruptions, a particularly useful stable genetic alteration is a gene deletion. The use of a gene deletion to introduce a stable genetic alteration is particularly useful to reduce the likelihood of a reversion to a phenotype prior to the genetic alteration. For example, if desired, stable growth-coupled production of a biochemical can be achieved, for example, by deletion of a gene encoding an enzyme catalyzing one or more reactions within a set of metabolic modifications. The stability of growth-coupled production of a biochemical can be further enhanced through multiple deletions, significantly reducing the likelihood of multiple compensatory reversions occurring for each disrupted activity.

Those skilled in the art will understand that the genetic alterations, including metabolic modifications exemplified herein are described with reference to a suitable source or host organism such as E. coli, yeast, or other organisms disclosed herein and their corresponding metabolic reactions or a suitable source organism for desired genetic material such as genes encoding enzymes for their corresponding metabolic reactions for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 1000% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production, including growth-coupled production, of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having 4-HB, GBL, 4-HBal, 4-HBCoA, BDO and/or putrescine biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes. Similarly for a gene disruption, evolutionally related genes can also be disrupted or deleted in a host microbial organism to reduce or eliminate functional redundancy of enzymatic activities targeted for disruption.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

“Conservative amino acid substitution” or, simply, “conservative variations” of a particular sequence refers to the replacement of one amino acid, or series of amino acids, with essentially identical amino acid sequences. One of skill will recognize that individual substitutions, deletions or additions that alter, add or delete a single amino acid or a percentage of amino acids in an encoded sequence result in “conservative variations” where the alterations result in the deletion of an amino acid, addition of an amino acid, or substitution of an amino acid with a functionally similar amino acid. One of skill in the art will also recognize that an individual substitution, deletion or addition disclosed herein that refers to a specific variant, e.g. substitution of a Glycine a residue 34 with a Histidine (G34H), can include a conservative substitution made at the same residue as disclosed herein, e.g. instead of a Histindine being substituted at residue 34, an Arginine (R) or a Lysine (K) can be substituted.

Conservative substitution tables providing functionally similar amino acids are well known in the art. Conservative amino acid substitutions includes replacement of one amino acid with another having the same type of functional group or side chain polarity, size, shape or charge (e.g., aliphatic, aromatic, positively charged, negatively charged, polar, non-polar, positive polar, negative polar, uncharged polar, non-polar hydrophobic, ionizable acidic, ionizable basic, or sulfur containing residues).

As a non-limiting example, the following six groups each contain amino acids that can be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K); Histidine (H);
5) Isoleucine (I), Leucine (L), Methionine (M), Valine (V); and
6) Phenylalanine (F), Tyrosine (Y), Tryptophan (W).

Thus, conservative amino acid substitutions of a polypeptide sequence disclosed herein can include substitutions of a percentage, such as less than 1%, 5% or 10%, of the amino acids of the polypeptide sequence, with a conservatively selected amino acid of the same conservative substitution group. Additionally, a conservatively substituted variation of a polypeptide sequence disclosed herein can contain no more than 1, 2, 3, 4, 5, 10, 20, 30, 50, or 100 substitutions with a conservatively substituted variation of the same conservative substitution group.

It is also understood that the addition or substitution of nucleic acid sequences which do not alter the encoded polypeptide activity of a nucleic acid molecule, such as the addition of a non-functional or non-coding sequence, is a conservative variation of the nucleic acid sequence. One of skill in the art will appreciate that many conservative variations of the nucleic acid molecules disclosed yield a functionally identical polypeptide. For example, owing to the degeneracy of the genetic code, silent substitutions (i.e., substitutions in a nucleic acid sequence which do not result in an alteration in an encoded polypeptide) are an implied feature of every nucleic acid sequence which encodes an amino acid. Similarly, conservative amino acid substitutions, in one or more amino acids in an amino acid sequence can be substituted with different amino acids with highly similar properties, are also readily identified as being highly similar to a disclosed construct. Such conservative variations of each disclosed sequence are a feature of the polypeptides disclosed herein.

Non-conservative modifications of a particular polypeptide are those which substitute any amino acid not characterized as a conservative substitution. For example, any substitution which crosses the bounds of the six groups set forth above. These include substitutions of basic or acidic amino acids for neutral amino acids (e.g., Aspartic acid (D), Glutamic acid (E), Asparagine (N), or Glutamine (Q) for Valine (V), Isoleucine (I), Leucine (L) or Methionine (M)), aromatic amino acid for basic or acidic amino acids (e.g., Phenylalanine (F), Tyrosine (Y) or Tryptophan (W) for Aspartic acid (D), Asparagine (N), Glutamic acid (E) or Glutamine (Q)), or any other substitution not replacing an amino acid with alike amino acid.

Disclosed herein are non-naturally occurring microbial biocatalyst or microbial organisms including a microbial organism having a 4-hydroxybutanoic acid (4-HB) biosynthetic pathway that includes at least one exogenous nucleic acid encoding 4-hydroxybutanoate dehydrogenase, CoA-independent succinic semialdehyde dehydrogenase, succinyl-CoA synthetase, CoA-dependent succinic semialdehyde dehydrogenase, glutamate:succinic semialdehyde transaminase, alpha-ketoglutarate decarboxylase, or glutamate decarboxylase, wherein the exogenous nucleic acid is expressed in sufficient amounts to produce monomeric 4-hydroxybutanoic acid (4-HB). 4-hydroxybutanoate dehydrogenase is also referred to as 4-hydroxybutyrate dehydrogenase or 4-HB dehydrogenase. Succinyl-CoA synthetase is also referred to as succinyl-CoA synthase or succinyl-CoA ligase.

Also disclosed herein is a non-naturally occurring microbial biocatalyst or microbial organism including a microbial organism having a 4-hydroxybutanoic acid (4-HB) biosynthetic pathway having at least one exogenous nucleic acid encoding 4-hydroxybutanoate dehydrogenase, succinyl-CoA synthetase, CoA-dependent succinic semialdehyde dehydrogenase, or α-ketoglutarate decarboxylase, wherein the exogenous nucleic acid is expressed in sufficient amounts to produce monomeric 4-hydroxybutanoic acid (4-HB).

The non-naturally occurring microbial biocatalysts or microbial organisms can include microbial organisms that employ combinations of metabolic reactions for biosynthetically producing the compounds of the invention. The biosynthesized compounds can be produced intracellularly and/or secreted into the culture medium. Exemplary compounds produced by the non-naturally occurring microorganisms include, for example, 4-hydroxybutanoic acid, 1,4-butanediol and γ-butyrolactone.

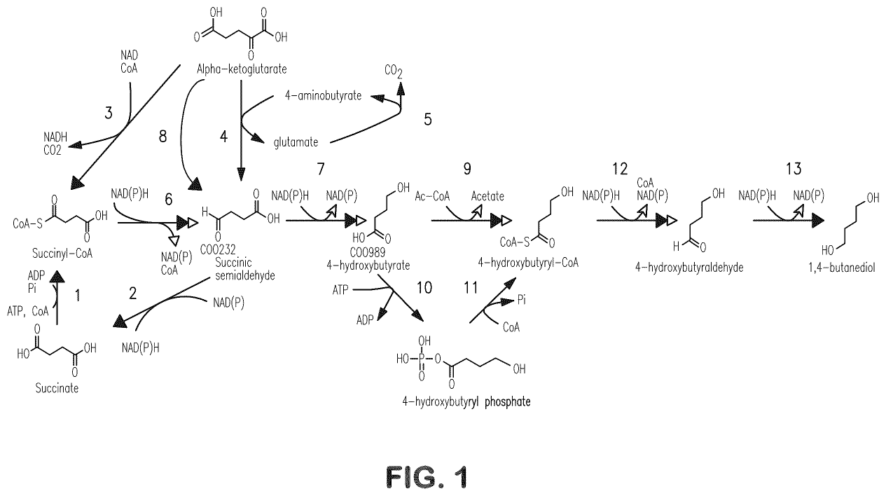

In one embodiment, a non-naturally occurring microbial organism is engineered to produce 4-HB. This compound is one useful entry point into the 1,4-butanediol family of compounds. The biochemical reactions for formation of 4-HB from succinate, from succinate through succinyl-CoA or from α-ketoglutarate are shown in steps 1-8 of FIG. 1.

It is understood that any combination of appropriate enzymes of a BDO, 4-HB, 4-HBal, 4-HBCoA and/or putrescine pathway can be used so long as conversion from a staffing component to the BDO, 4-HBal, 4-HBCoA and/or putrescine product is achieved. Thus, it is understood that any of the metabolic pathways disclosed herein can be utilized and that it is well understood to those skilled in the art how to select appropriate enzymes to achieve a desired pathway, as disclosed herein.

In another embodiment, disclosed herein is a non-naturally occurring microbial organism, comprising a microbial organism having a 1,4-butanediol (BDO) pathway comprising at least one exogenous nucleic acid encoding a BDO pathway enzyme expressed in a sufficient amount to produce BDO, the BDO pathway comprising 4-aminobutyrate CoA transferase, 4-aminobutyryl-CoA hydrolase, 4-aminobutyrate-CoA ligase, 4-aminobutyryl-CoA oxidoreductase (deaminating), 4-aminobutyryl-CoA transaminase, or 4-hydroxybutyryl-CoA dehydrogenase (see Example VII; see corresponding Example and Table 17 in WO2013/184602). The BDO pathway further can comprise 4-hydroxybutyryl-CoA reductase (alcohol forming), 4-hydroxybutyryl-CoA reductase, or 1,4-butanediol dehydrogenase.

It is understood by those skilled in the art that various combinations of the pathways can be utilized, as disclosed herein. For example, in a non-naturally occurring microbial organism, the nucleic acids can encode 4-aminobutyrate CoA transferase, 4-aminobutyryl-CoA hydrolase, or 4-aminobutyrate-CoA ligase; 4-aminobutyryl-CoA oxidoreductase (deaminating) or 4-aminobutyryl-CoA transaminase; and 4-hydroxybutyryl-CoA dehydrogenase. Other exemplary combinations are specifically describe below and further can be found in FIGS. 8-13. For example, the BDO pathway can further comprise 4-hydroxybutyryl-CoA reductase (alcohol forming), 4-hydroxybutyryl-CoA reductase, or 1,4-butanediol dehydrogenase.

Additionally disclosed herein is a non-naturally occurring microbial organism, comprising a microbial organism having a BDO pathway comprising at least one exogenous nucleic acid encoding a BDO pathway enzyme expressed in a sufficient amount to produce BDO, the BDO pathway comprising 4-aminobutyrate CoA transferase, 4-aminobutyryl-CoA hydrolase, 4-aminobutyrate-CoA ligase, 4-aminobutyryl-CoA reductase (alcohol forming), 4-aminobutyryl-CoA reductase, 4-aminobutan-1-ol dehydrogenase, 4-aminobutan-1-ol oxidoreductase (deaminating) or 4-aminobutan-1-ol transaminase (see Example VII; see corresponding Example and Table 18 in WO2013/184602), and can further comprise 1,4-butanediol dehydrogenase. For example, the exogenous nucleic acids can encode 4-aminobutyrate CoA transferase, 4-aminobutyryl-CoA hydrolase, or 4-aminobutyrate-CoA ligase; 4-aminobutyryl-CoA reductase (alcohol forming); and 4-aminobutan-1-ol oxidoreductase (deaminating) or 4-aminobutan-1-ol transaminase. In addition, the nucleic acids can encode. 4-aminobutyrate CoA transferase, 4-aminobutyryl-CoA hydrolase, or 4-aminobutyrate-CoA ligase; 4-aminobutyryl-CoA reductase; 4-aminobutan-1-ol dehydrogenase; and 4-aminobutan-1-ol oxidoreductase (deaminating) or 4-aminobutan-1-ol transaminase.

Also disclosed herein is a non-naturally occurring microbial organism, comprising a microbial organism having a BDO pathway comprising at least one exogenous nucleic acid encoding a BDO pathway enzyme expressed in a sufficient amount to produce BDO, the BDO pathway comprising 4-aminobutyrate kinase, 4-aminobutyraldehyde dehydrogenase (phosphorylating), 4-aminobutan-1-ol dehydrogenase, 4-aminobutan-1-ol oxidoreductase (deaminating), 4-aminobutan-1-ol transaminase, [(4-aminobutanolyl)oxy]phosphonic acid oxidoreductase (deaminating), [(4-aminobutanolyl)oxy]phosphonic acid transaminase, 4-hydroxybutyryl-phosphate dehydrogenase, or 4-hydroxybutyraldehyde dehydrogenase (phosphorylating)(see Example VII; see corresponding Example and Table 19 in WO2013/184602). For example, the exogenous nucleic acids can encode 4-aminobutyrate kinase; 4-aminobutyraldehyde dehydrogenase (phosphorylating); 4-aminobutan-1-ol dehydrogenase; and 4-aminobutan-1-ol oxidoreductase (deaminating) or 4-aminobutan-1-ol transaminase. Alternatively, the exogenous nucleic acids can encode 4-aminobutyrate kinase; [(4-aminobutanolyl)oxy]phosphonic acid oxidoreductase (deaminating) or [(4-aminobutanolyl)oxy]phosphonic acid transaminase; 4-hydroxybutyryl-phosphate dehydrogenase; and 4-hydroxybutyraldehyde dehydrogenase (phosphorylating).

Additionally disclosed herein is a non-naturally occurring microbial organism, comprising a microbial organism having a BDO pathway comprising at least one exogenous nucleic acid encoding a BDO pathway enzyme expressed in a sufficient amount to produce BDO, the BDO pathway comprising alpha-ketoglutarate 5-kinase, 2,5-dioxopentanoic semialdehyde dehydrogenase (phosphorylating), 2,5-dioxopentanoic acid reductase, alpha-ketoglutarate CoA transferase, alpha-ketoglutaryl-CoA hydrolase, alpha-ketoglutaryl-CoA ligase, alpha-ketoglutaryl-CoA reductase, 5-hydroxy-2-oxopentanoic acid dehydrogenase, alpha-ketoglutaryl-CoA reductase (alcohol forming), 5-hydroxy-2-oxopentanoic acid decarboxylase, or 5-hydroxy-2-oxopentanoic acid dehydrogenase (decarboxylation)(see Example VIII; see corresponding Example and Table 20 in WO2013/184602). The BDO pathway can further comprise 4-hydroxybutyryl-CoA reductase (alcohol forming), 4-hydroxybutyryl-CoA reductase, or 1,4-butanediol dehydrogenase.

Further disclosed herein is a non-naturally occurring microbial organism, comprising a microbial organism having a BDO pathway comprising at least one exogenous nucleic acid encoding a BDO pathway enzyme expressed in a sufficient amount to produce BDO, the BDO pathway comprising glutamate CoA transferase, glutamyl-CoA hydrolase, glutamyl-CoA ligase, glutamate 5-kinase, glutamate-5-semialdehyde dehydrogenase (phosphorylating), glutamyl-CoA reductase, glutamate-5-semialdehyde reductase, glutamyl-CoA reductase (alcohol forming), 2-amino-5-hydroxypentanoic acid oxidoreductase (deaminating), 2-amino-5-hydroxypentanoic acid transaminase, 5-hydroxy-2-oxopentanoic acid decarboxylase, 5-hydroxy-2-oxopentanoic acid dehydrogenase (decarboxylation)(see Example IX; see corresponding Example and Table 21 in WO2013/184602).

Also disclosed herein is a non-naturally occurring microbial organism, comprising a microbial organism having a BDO pathway comprising at least one exogenous nucleic acid encoding a BDO pathway enzyme expressed in a sufficient amount to produce BDO, the BDO pathway comprising 3-hydroxybutyryl-CoA dehydrogenase, 3-hydroxybutyryl-CoAdehydratase, vinylacetyl-CoA Δ-isomerase, or 4-hydroxybutyryl-CoA dehydratase (see Example X; see corresponding Example and Table 22 in WO2013/184602). For example, the exogenous nucleic acids can encode 3-hydroxybutyryl-CoA dehydrogenase; 3-hydroxybutyryl-CoA dehydratase; vinylacetyl-CoA Δ-isomerase; and 4-hydroxybutyryl-CoA dehydratase.

Further disclosed herein is a non-naturally occurring microbial organism, comprising a microbial organism having a BDO pathway comprising at least one exogenous nucleic acid encoding a BDO pathway enzyme expressed in a sufficient amount to produce BDO, the BDO pathway comprising homoserine deaminase, homoserine CoA transferase, homoserine-CoA hydrolase, homoserine-CoA ligase, homoserine-CoA deaminase, 4-hydroxybut-2-enoyl-CoA transferase, 4-hydroxybut-2-enoyl-CoA hydrolase, 4-hydroxybut-2-enoyl-CoA ligase, 4-hydroxybut-2-enoate reductase, 4-hydroxybutyryl-CoA transferase, 4-hydroxybutyryl-CoA hydrolase, 4-hydroxybutyryl-CoA ligase, or 4-hydroxybut-2-enoyl-CoA reductase (see Example XI; see corresponding Example and Table 23 in WO2013/184602).

Further disclosed herein is a non-naturally occurring microbial organism, comprising a microbial organism having a BDO pathway comprising at least one exogenous nucleic acid encoding a BDO pathway enzyme expressed in a sufficient amount to produce BOD, the BDO pathway comprising succinyl-CoA reductase (alcohol forming), 4-hydroxybutyryl-CoA hydrolase, 4-hydroxybutyryl-CoA ligase, 4-hydroxybutanal dehydrogenase (phosphorylating) (see Table 15). Such a BDO pathway can further comprise succinyl-CoA reductase, 4-hydroxybutyrate dehydrogenase, 4-hydroxybutyryl-CoA transferase, 4-hydroxybutyrate kinase, phosphotrans-4-hydroxybutyrylase, 4-hydroxybutyryl-CoA reductase, 4-hydroxybutyryl-CoA reductase (alcohol forming), or 1,4-butanediol dehydrogenase.

Additionally disclosed herein is a non-naturally occurring microbial organism, comprising a microbial organism having a BDO pathway comprising at least one exogenous nucleic acid encoding a BDO pathway enzyme expressed in a sufficient amount to produce BDO, the BDO pathway comprising glutamate dehydrogenase, 4-aminobutyrate oxidoreductase (deaminating), 4-aminobutyrate transaminase, glutamate decarboxylase, 4-hydroxybutyryl-CoA hydrolase, 4-hydroxybutyryl-CoA ligase, 4-hydroxybutanal dehydrogenase (phosphorylating)(see Table 16). Such a BDO pathway can further comprise alpha-ketoglutarate decarboxylase, 4-hydroxybutyrate dehydrogenase, 4-hydroxybutyryl-CoA transferase, 4-hydroxybutyrate kinase, phosphotrans-4-hydroxybutyrylase, 4-hydroxybutyryl-CoA reductase, 4-hydroxybutyryl-CoA reductase (alcohol forming), or 1,4-butanediol dehydrogenase.

The pathways described above are merely exemplary. One skilled in the art can readily select appropriate pathways from those disclosed herein to obtain a suitable BDO pathway or other metabolic pathway, as desired.

The invention provides genetically modified organisms that allow improved production of a desired product such as BDO by increasing the product or decreasing undesirable byproducts. As disclosed herein, the invention provides a non-naturally occurring microbial organism, comprising a microbial organism having a 1,4-butanediol (BDO) pathway comprising at least one exogenous nucleic acid encoding a BDO pathway enzyme expressed in a sufficient amount to produce BDO. In one embodiment, the microbial organism is genetically modified to express exogenous succinyl-CoA synthetase (see Example XII). For example, the succinyl-CoA synthetase can be encoded by an Escherichia coli sucCD genes.

In another embodiment, the microbial organism is genetically modified to express exogenous alpha-ketoglutarate decarboxylase (see Example XIII). For example, the alpha-ketoglutarate decarboxylase can be encoded by the Mycobacterium bovis sucA gene. In still another embodiment, the microbial organism is genetically modified to express exogenous succinate semialdehyde dehydrogenase and 4-hydroxybutyrate dehydrogenase and optionally 4-hydroxybutyryl-CoA/acetyl-CoA transferase (see Example XIII). For example, the succinate semialdehyde dehydrogenase (CoA-dependent), 4-hydroxybutyrate dehydrogenase and 4-hydroxybutyryl-CoA/acetyl-CoA transferase can be encoded by Porphyromonas gingivalis W83 genes. In an additional embodiment, the microbial organism is genetically modified to express exogenous butyrate kinase and phosphotransbutyrylase (see Example XIII). For example, the butyrate kinase and phosphotransbutyrylase can be encoded by Clostridium acetobutilicum buk1 and ptb genes.

In yet another embodiment, the microbial organism is genetically modified to express exogenous 4-hydroxybutyryl-CoA reductase (see Example XIII). For example, the 4-hydroxybutyryl-CoA reductase can be encoded by Clostridium beijerinckii ald gene. Additionally, in an embodiment of the invention, the microbial organism is genetically modified to express exogenous 4-hydroxybutanal reductase (see Example XIII). For example, the 4-hydroxybutanal reductase can be encoded by Geobacillus thermoglucosidasius adh1 gene. In another embodiment, the microbial organism is genetically modified to express exogenous pyruvate dehydrogenase subunits (see Example XIV). For example, the exogenous pyruvate dehydrogenase can be NADH insensitive. The pyruvate dehydrogenase subunit can be encoded by the Klebsiella pneumonia lpdA gene. In a particular embodiment, the pyruvate dehydrogenase subunit genes of the microbial organism can be under the control of a pyruvate formate lyase promoter.

In still another embodiment, the microbial organism is genetically modified to disrupt a gene encoding an aerobic respiratory control regulatory system (see Example XV). For example, the disruption can be of the arcA gene. Such an organism can further comprise disruption of a gene encoding malate dehydrogenase. In a further embodiment, the microbial organism is genetically modified to express an exogenous NADH insensitive citrate synthase (see Example XV). For example, the NADH insensitive citrate synthase can be encoded by gltA, such as an R163L mutant of gltA. In still another embodiment, the microbial organism is genetically modified to express exogenous phosphoenolpyruvate carboxykinase (see Example XVI). For example, the phosphoenolpyruvate carboxykinase can be encoded by an Haemophilus influenza phosphoenolpyruvate carboxykinase gene.

In an additional embodiment, the invention provides a non-naturally occurring microbial organism having a 4-hydroxybutyrate pathway and being capable of producing 4-hydroxybutyrate, wherein the microbial organism comprises a genetic modification that increases expression of phosphoenolpyruvate carboxylase (see Example XXXI). In such an embodiment in which phosphoenolpyruvate expression is increased, the microbial organism can exhibit decreased production of ethanol, acetate, pyruvate or alanine, or a combination thereof, relative to a parent microbial organism in the absence of the genetic modification. As disclosed herein, the overexpression of phosphoenolpyruvate (PEP) carboxylase leads to conversion of more phosphoenolpyruvate into oxaloacetate, thereby reducing flux from PEP into pyruvate and subsequently into acetyl-CoA (Example XXXI). Reducing flux from PEP to pyruvate and acetyl-CoA increases flux into the TCA cycle and consequently a 4HB or BDO pathway.

In yet another embodiment, the invention provides a non-naturally occurring microbial organism having a 4-hydroxybutyrate pathway and being capable of producing 4-hydroxybutyrate, wherein the microbial organism comprises a genetic modification that increases expression of alpha-ketoglutarate dehydrogenase (see Example XXXII). The increased expression of alpha-ketoglutarate dehydrogenase can result in decreased production of glutamate relative to a parent microbial organism in the absence of the genetic modification. Such a microbial organism can also exhibit decreased production of ethanol, acetate, pyruvate or alanine, or a combination thereof, relative to a parent microbial organism in the absence of the genetic modification. As disclosed herein, the formation of glutamate can lead to a carbon loss and reduction in yield. Therefore, increased expression of alpha-ketoglutarate dehydrogenase can reduce glutamate as well as other by-products such as ethanol, acetate, alanine and/or pyruvate.

In a further embodiment, the invention provides a non-naturally occurring microbial organism having a 4-hydroxybutyrate pathway and being capable of producing 4-hydroxybutyrate, wherein the microbial organism comprises a genetic modification that increases expression of a non-phosphotransferase (PTS) glucose uptake system (see Example XXXIII). In such an embodiment, the genetic modification can comprise increased expression of a permease, glucokinase, or a glucose facilitator, or a combination thereof. In addition, such a microbial organism can exhibit decreased production of ethanol, acetate, pyruvate, or alanine, or a combination thereof, relative to a parent microbial organism in the absence of the genetic modification. The introduction of a non-PTS sucrose uptake system has been previously described in U.S. publication 2011/0045575 as a way to reduce pyruvate formation in a microbial organism utilizing sucrose as a carbon source. In contrast, the utilization of a non-PTS glucose uptake system, as described herein, is to provide a better balance between the available oxaloacetate in comparison to acetyl-CoA (see Example XXXIII).

In another embodiment, the invention provides a non-naturally occurring microbial organism having a 4-hydroxybutyrate pathway and being capable of producing 4-hydroxybutyrate, wherein the microbial organism comprises a genetic modification that increases expression of a gamma-butyrolactone esterase (Example XXXIV). Such a microbial organism can exhibit decreased production of gamma-butyrolactone relative to a parent microbial organism in the absence of the genetic modification. As disclosed herein, gamma-butyrolactone is a byproduct formed during the fermentation of sugars to 1,4-butanediol. Gamma-butyrolactone can form from the unstable pathway intermediate 4-hydroxybutyryl-CoA as well as spontaneous lactonization of 4-hydroxybutyrate. As disclosed herein, the expression of a gamma-butyrolactone can accelerate the hydrolysis of gamma-butyrolactone to BDO, thereby improving BDO product yield and eliminating a byproduct (see Example XXXIV).

In additional embodiments disclosed herein, a genetic modification can include gene disruptions or deletions to decrease expression of an enzyme. For example, in a further embodiment, the invention provides a non-naturally occurring microbial organism having a 4-hydroxybutyrate pathway and being capable of producing 4-hydroxybutyrate, wherein the microbial organism comprises a genetic modification that decreases expression of succinyl-CoA synthetase (Example XXXV). In such an embodiment, the microbial organism can exhibit increased production of 4-hydroxybutyrate relative to a parent microbial organism in the absence of the genetic modification. As described herein, repeated rounds of flux through the TCA cycle results in carbon loss as CO₂. The deletion of succinyl-CoA synthetase blocks the TCA cycle downstream of succinyl-CoA, thereby reducing CO₂ loss (see Example XXXV).

In a further embodiment, the invention provides a non-naturally occurring microbial organism having a 4-hydroxybutyrate pathway and being capable of producing 4-hydroxybutyrate, wherein the microbial organism comprises a genetic modification that decreases expression of an acyl coenzyme A thioesterase (see Example XXXVI). In such an embodiment the microbial organism can exhibit decreased production of gamma-butyrolactone relative to a parent microbial organism in the absence of the genetic modification. In yet a further embodiment, such a microbial organism can comprise at least two genetic modifications that decrease expression of at least two acyl coenzyme A thioesterases. As disclosed herein, the BDO pathway intermediate 4-hydroxybutyryl-CoA spontaneously and enzymatically cyclizes to form gamma-butyrolactone (GBL). By deleting acyl coenzyme-A thioesterases, the formation of the byproduct GBL was reduced (see Example XXXVI).

In another embodiment, the invention provides a non-naturally occurring microbial organism having a 4-hydroxybutyrate pathway and being capable of producing 4-hydroxybutyrate, wherein the microbial organism comprises a genetic modification that decreases expression of an alcohol dehydrogenase (see Example XXXVII). In such an embodiment, the microbial organism can exhibit decreased backflux from a downstream product of the 4-hydroxybutyrate pathway relative to a parent microbial organism in the absence of said genetic modification. At high titers of BDO, a downstream product from 4-hydroxybutyrate, the high concentrations of product can result in backflux within the pathway or through side reactions, thereby resulting in a decreased product yield and/or an increase in toxic byproduct formation such as aldehydes. As described herein, several endogenous alcohol dehydrogenases were found to contribute to backflux, whereas deletion of one or more of the endogenous alcohol dehydrogenases decreased backflux without reducing production of 4-hydroxybutyrate or BDO, and in fact increased BDO formation (see Example XXXVII).

In still another embodiment, the invention provides a non-naturally occurring microbial organism having a 4-hydroxybutyrate pathway and being capable of producing 4-hydroxybutyrate, wherein the microbial organism comprises a genetic modification that decreases expression of a non-energy-producing NADH dehydrogenase (see Example XXXVIII). The decreased expression of a non-energy-producing NADH dehydrogenase suppresses depletion of the NADH pool resulting from the activity of the non-energy-producing NADH dehydrogenase. Additionally, the microbial organisms exhibit increased energy efficiency in the microbial organism relative to a parent microbial organism in the absence of the genetic modification. As described herein, the electron transport chain has multiple NADH dehydrogenases and cytochrome oxidases with varying ability to translocate protons. Some NADH dehydrogenases consume NADH without linking the consumption to proton translocation and energy production, and such NADH dehydrogenases are considered to be non-energy-producing NADH dehydrogenases. One exemplary non-energy-producing NADH dehydrogenase is NADH II of E. coli (see Example XXXVIII). Additional genes encoding enzymes with non-energy producing NAD(P)H dehydrogenase activities include wrbA, yieF, and kefF. One skilled in the art will readily understand the meaning of a non-energy-producing NADH dehydrogenase as one that does not couple NADH oxidation to electron transport and proton translocation and the formation of ATP By decreasing expression of a non-energy-producing NADH dehydrogenase, the depletion of the NADH pool within the cell is suppressed, thereby making the cell more energy efficient and/or allowing NADH to be utilized in desired synthetic pathways.

In a further embodiment, the invention provides a non-naturally occurring microbial organism having a 4-hydroxybutyrate pathway and being capable of producing 4-hydroxybutyrate, wherein the microbial organism comprises a genetic modification that decreases expression of a cytochrome oxidase (see Example XXXIX). In such an embodiment, the microbial organism can exhibit increased energy efficiency relative to a parent microbial organism in the absence of the genetic modification. As disclosed herein, cytochrome oxidases involved in the electron transport chain have different energy-conserving efficiencies. By decreasing expression of one or more cytochrome oxidases, the energy efficiency of the cell can be increased (see Example XXXIX). Even in a case where an increase in product yield is not observed under certain conditions (see Example XXXIX), such genetic modifications can be advantageous in providing a greater tolerance for a range of oxygen concentrations, in particular producing a product more efficiently in a large fermentor, where the available oxygen varies within the fermentor. By improving the energy efficiency of the microbial organism, the organism can tolerate lower oxygen conditions since need for energy production from the electron transport chain is reduced. Thus, in a particular embodiment of the invention, the microbial organism can exhibit an increased tolerance to a range of oxygen concentrations relative to a parent microbial organism in the absence of said genetic modification.

The invention thus provides a non-naturally occurring microbial organism having a 4-hydroxybutyrate pathway and being capable of producing 4-hydroxybutyrate, wherein the microbial organism comprises a genetic modification, the genetic modification selected from: (A) a genetic modification that increases expression of acetate kinase and phosphotransacetylase; (B) a genetic modification that inactivates expression of an aerobic respiratory control regulatory system; (C) a genetic modification that truncates a glucose phosphotransferase system (PTS) permease; (D) a genetic modification that increases expression of phosphoenolpyruvate carboxylase; (E) a genetic modification that increases expression of alpha-ketoglutarate dehydrogenase; (F) a genetic modification that increases expression of a non-phosphotransferase (PTS) glucose uptake system; (G) a genetic modification that increases expression of a gamma-butyrolactone esterase; (H) a genetic modification that decreases expression of succinyl-CoA synthetase; (I) a genetic modification that decreases expression of an acyl coenzyme A thioesterase; (J) a genetic modification that decreases expression of an alcohol dehydrogenase; (K) a genetic modification that decreases expression of a non-energy-producing NADH dehydrogenase; (L) a genetic modification that decreases expression of a cytochrome oxidase; and (M) a combination of two or more, for example, three or more, four or more, five or more, six or more, seven or more, eight or more, or nine or more, ten or more, eleven or more, or all of the genetic modifications of parts (A)-(L).

In another embodiment, the invention provides a microbial organism comprising a genetic modification, where the genetic modification is selected from: (A) a genetic modification that increases expression of acetate kinase and phosphotransacetylase; (B) a genetic modification that inactivates expression of an aerobic respiratory control regulatory system; (C) a genetic modification that truncates a glucose phosphotransferase system (PTS) permease; and (N) a combination of two or more, or all of the genetic modifications of parts (A)-(C). In a further embodiment, such a microbial organism can further comprise a genetic modification is selected from: (D) a genetic modification that increases expression of phosphoenolpyruvate carboxylase; (E) a genetic modification that increases expression of alpha-ketoglutarate dehydrogenase; (F) a genetic modification that increases expression of a non-phosphotransferase (PTS) glucose uptake system; (G) a genetic modification that increases expression of a gamma-butyrolactone esterase; (H) a genetic modification that decreases expression of succinyl-CoA synthetase; (I) a genetic modification that decreases expression of an acyl coenzyme A thioesterase; (J) a genetic modification that decreases expression of an alcohol dehydrogenase; (K) a genetic modification that decreases expression of a non-energy-producing NADH dehydrogenase; (L) a genetic modification that decreases expression of a cytochrome oxidase; and (M) a combination of two or more, three or more, four or more, five or more, six or more, seven or more, eight or more, nine or more, ten or more, eleven or more, or all of the genetic modifications of parts (A)-(L).

In further particular embodiments, (a) the microbial organism of part (A) has increased conversion of acetate to acetyl-CoA and/or reduced secretion of acetate; (b) the microbial organism of part (B) has an inactivation mutation in arcA by an insertion in the coding sequence; (c) the microbial organism of part (C) has reduced glucose uptake and/or decreased production of acetate, pyruvate and/or glutamate; (d) the microbial organism of part (D), (E), or (F) has decreased production of ethanol, acetate, pyruvate or alanine, or a combination thereof, relative to a parent microbial organism in the absence of said genetic modification; (e) the microbial organism of part (E) has decreased production of glutamate relative to a parent microbial organism in the absence of said genetic modification; (f) the microbial organism of part (F) has a genetic modification comprising increased expression of a permease, glucokinase, or a glucose facilitator, or a combination thereof, (g) the microbial organism of part (G) has decreased production of gamma-butyrolactone relative to a parent microbial organism in the absence of said genetic modification; (h) the microbial organism of part (I) has increased production of 4-hydroxybutyrate relative to a parent microbial organism in the absence of said genetic modification; (i) the microbial organism of part (I) has decreased production of gamma-butyrolactone relative to a parent microbial organism in the absence of said genetic modification; (j) the microbial organism of part (I) has a genetic modification comprising at least two genetic modifications that decrease expression of at least two acyl coenzyme A thioesterases; (k) the microbial organism of part (J) has decreased backflux from a downstream product of the 4-hydroxybutyrate pathway relative to a parent microbial organism in the absence of said genetic modification; (l) the microbial organism of part (K) has suppressed depletion of the NADH pool or increased energy efficiency in the microbial organism, or a combination thereof, relative to a parent microbial organism in the absence of said genetic modification; (m) the microbial organism of part (L) has increased energy efficiency relative to a parent microbial organism in the absence of said genetic modification; or (n) the microbial organism of part (L) has increased tolerance to a range of oxygen concentrations relative to a parent microbial organism in the absence of said genetic modification.

In a further embodiment, the invention provides a microbial organism, where the microbial organism of parts (G) or (I) further comprises a 4-hydroxybutyryl-CoA pathway. Additional metabolic modifications include, but are not limited to, a genetic modification that increases expression of acetate kinase and/or phosphotransacetylase (see Example XXXX), a genetic modification that decreases expression of arcA, in particular an insertion mutation (see Example XXXXI), or a genetic modification in which a glucose permease is a modified variant such as a truncation (see Example XXXXII). The invention further provides a method for producing 4-hydroxybutyrate utilizing such microbial organisms. It is understood that such genetic modifications include, but are not limited to, the specifically described gene additions and disruptions described in Examples XXIV-XXXXII. It is understood that the genetic modifications described herein can be used alone or in combination with other genetic modifications, particularly those disclosed herein, as desired to provide desirable characteristics of a production strain. In the case of gene disruptions, it is understood that such disruptions involve disruption of an endogenous gene encoding an activity of a corresponding gene product to be decreased.

In still a further embodiment, a microbial organism of the invention can further comprise a metabolic modification selected from: (i) a genetic modification that increases expression of pyruvate dehydrogenase; (ii) a genetic modification that decreases expression of an aerobic respiratory control regulatory system; (iii) a genetic modification that increases expression of an NADH insensitive citrate synthase; (iv) a genetic modification that decreases expression of malate dehydrogenase; (v) a genetic modification that decreases expression of lactate dehydrogenase; (vi) a genetic modification that decreases expression of alcohol dehydrogenase; (vii) a genetic modification that decreases expression of pyruvate formate lyase; (viii) a genetic modification that increases expression of phosphoenolpyruvate carboxykinase; and (ix) a combination of two or more, three or more, four or more, five or more, six or more, seven or more, or all of the genetic modifications of parts (i)-(viii).

Although the pathways immediately above are directed to 4-HB pathways, it is understood, as disclosed herein, the 4-HB is a precursor to downstream products such as 1,4-butanediol (BDO). Further, as disclosed herein, a microbial organism having a 4-HB pathway can further include enzymes that convert 4-HB to a downstream product such as BDO, as desired. In addition, any of the pathways described herein that produce 4-HB are understood to provide a 4-HB pathway, even if additional steps in a pathway are also disclosed since such a pathway produces 4-HB. Thus, the invention provides microbial organisms comprising a 4-HB pathway or a BDO pathway, wherein the organism comprises one or more of the genetic modifications disclosed herein and produces 4-HB, 4-HB-CoA, or BDO, as well as methods of producing products such as 4-HB, 4-HB-CoA and/or BDO using such an organism. It is further understood that the genetic modifications can be used alone or in various combinations of one or more of the genetic modifications, as disclosed herein (see Examples). Additionally, it is understood that a genetic modification can result in a desired effect, including but not limited to increased product yield, decreased byproduct production, in particular toxic or growth suppressive byproducts, for example, aldehydes, improved strain culture growth or fermentation characteristics, improved ability to isolate or purify a desired product, and the like. Furthermore, it is understood that increased production of a downstream product, for example, BDO, can be indicative of increased production of an upstream product, for example, a pathway intermediate such as 4HB, where increased production of the downstream product indicates increased flux through an upstream product. One skilled in the art can readily understand these or other desirable characteristics of a microbial organism of the invention for producing a desired pathway product or intermediate. Thus, using methods disclosed herein and those well known in the art, one skilled in the art can incorporate various metabolic modifications, incorporate appropriate expression control elements and/or methods, and/or variant enzymes to provide a microbial organism having optimized production properties. It is further understood that such optimized production properties can be readily determined by those skilled in the art, including increasing or decreasing the expression level of a particular molecule, including metabolic modifications and/or enzymes or proteins of a 4-HB, 4-HBal, 4-HBCoA, BDO or putrescine pathway.

As used herein, the term “parent microbial organism,” when used in the context of a genetic modification, is understood to mean a parent organism or strain in which the particular genetic modification has not been made but otherwise has the same genetic makeup. For example, if a strain of microbial organism is used to make a genetic modification that increases expression of a gene product, the parent strain would be the starting strain into which the heterologous gene is introduced. Similarly, a parent microbial organism of an organism in which a genetic modification has been made to decrease expression, such as a gene disruption or gene deletion, the parent microbial organism would have the same genetic background except for the gene disruption or deletion. However, it is understood that a parent microbial organism can differ by more than one genetic modification, either gene addition and/or disruption, depending on whether a single or multiple genetic modifications are being considered. One skilled in the art will readily understand the meaning of such a parent microbial organism in that the microbial organism is considered to be an appropriate control, as understood in the art, for observing the effect of one or more genetic modifications.

In addition, it is understood by those skilled in the art that a genetic modification such as those disclosed herein can contribute to an increase in product yield, a decrease in by-product formation, and/or improved characteristics of the microbial organism. Such improved characteristics include, but are not limited to, improved cell growth, increased energy efficiency, increased tolerance of a range of oxygen concentrations, particularly when manifested in a scaled up production process such as in large fermentors. A useful genetic modification may not alone, when compared to a parent microbial organism, exhibit an effect that can readily measured under a given set of conditions. However, a useful genetic modification can be advantageous if the modification on its on under a given set of conditions and/or in combination with one or more other genetic modifications is beneficial to the growth and/or production characteristics of the microbial organism.

It is understood that a genetic modification that increases expression of a desired enzyme is generally carried out by introducing into the microbial organism a heterologous nucleic acid encoding the desired enzyme. However, it is understood, as described herein, that a metabolic modification to increase expression can include modification of a regulatory region of an endogenous gene, such as a promoter and/or enhancer. Additionally, a genetic modification that decreases expression of a desired enzyme will generally involve a gene disruption or deletion, although modification of a regulatory to decrease expression can also be utilized, as disclosed herein.

It is further understood that any of a number of genetic modifications, as disclosed herein, can be used alone or in various combinations of one or more of the genetic modifications disclosed herein to increase the production of BDO in a BDO producing microbial organism. In a particular embodiment, the microbial organism can be genetically modified to incorporate any and up to all of the genetic modifications that lead to increased production of BDO. In a particular embodiment, the microbial organism containing a BDO pathway can be genetically modified to express exogenous succinyl-CoA synthetase; to express exogenous alpha-ketoglutarate decarboxylase; to express exogenous succinate semialdehyde dehydrogenase and 4-hydroxybutyrate dehydrogenase and optionally 4-hydroxybutyryl-CoA/acetyl-CoA transferase; to express exogenous butyrate kinase and phosphotransbutyrylase; to express exogenous 4-hydroxybutyryl-CoA reductase; and to express exogenous 4-hydroxybutanal reductase; to express exogenous pyruvate dehydrogenase; to disrupt a gene encoding an aerobic respiratory control regulatory system; to express an exogenous NADH insensitive citrate synthase; and to express exogenous phosphoenolpyruvate carboxykinase. Such strains for improved production are described in Examples XII-XIX. It is thus understood that, in addition to the modifications described above, such strains can additionally include other modifications disclosed herein. Such modifications include, but are not limited to, deletion of endogenous lactate dehydrogenase (ldhA), alcohol dehydrogenase (adhE), and/or pyruvate formate lyase (pflB) (see Examples XII-XIX and Table 18). Additional genetic modifications that can be introduced into a microbial organism include those described in Examples XXIV-XXXX, alone or in combination, including in combination with other genetic modifications disclosed herein.

The invention additionally provides a microbial organism that, in addition to one or more of the metabolic modifications of (A)-(J) described above, optionally further comprises a metabolic modification selected from (i) a genetic modification that increases expression of pyruvate dehydrogenase; (ii) a genetic modification that decreases expression of an aerobic respiratory control regulatory system; (iii) a genetic modification that increases expression of an NADH insensitive citrate synthase; (iv) a genetic modification that decreases expression of malate dehydrogenase; (v) a genetic modification that decreases expression of lactate dehydrogenase; (vi) a genetic modification that decreases expression of alcohol dehydrogenase; (vii) a genetic modification that decreases expression of pyruvate formate lyase; and (viii) a combination of two or more, three or more, four or more, five or more, six or more, or all of the genetic modifications of parts (i)-(vii). It is understood that any of the various combinations of metabolic modifications disclosed herein can be combined with each other, or with individual metabolic modifications A-P, to generate further combinations.

Additionally provided is a microbial organism in which one or more genes encoding the exogenously expressed enzymes are integrated into the fimD locus of the host organism (see Example XVII). For example, one or more genes encoding a BDO, 4-HB, 4-HBal, 4-HBCoA and/or putrescine pathway enzyme can be integrated into the fimD locus for increased production of BDO, 4-HB, 4-HBal, 4-HBCoA and/or putrescine. Further provided is a microbial organism expressing a non-phosphotransferase sucrose uptake system that increases production of BDO.

Although the genetically modified microbial organisms disclosed herein are exemplified with microbial organisms containing particular BDO, 4-HB, 4-HBal, 4-HBCoA and/or putrescine pathway enzymes, it is understood that such modifications can be incorporated into any microbial organism having a BDO, 4-HB, 4-HBal, 4-HBCoA and/or putrescine pathway suitable for enhanced production in the presence of the genetic modifications of any of the pathways disclosed herein. The microbial organisms of the invention can thus have any of the BDO, 4-HB, 4-HBal, 4-HBCoA and/or putrescine pathways disclosed herein (see, for example, FIGS. 1, 8-13, 58, 62, 63, 69 and 70). For example, if a depicted pathway shows a BDO pathway, whereas 4-hydroxybutyrate is an intermediate of the BDO pathway, it is understood, as disclosed herein, that a pathway to 4-hydroxybutyrate is also provided, if desired. Thus, the pathways described herein can produce an end product of a depicted pathway or a desired intermediate of a depicted pathway, as described herein. It is further understood that a pathway enzyme can be a variant enzyme, including but not limited to the variant enzymes disclosed herein (see Example XXXXII) so long as the pathway produces a desired pathway end product or pathway intermediate.

In an embodiment of the invention, a microbial organism is provided comprising a metabolic modification and a pathway to a desired product such as BDO, 4-HB, 4-HBal, 4-HBCoA and/or putrescine. Exemplary pathways are disclosed herein and include the pathways depicted in FIGS. 1, 8-13, 58, 62, 63, 69 and 70. In one embodiment, the microbial organism comprises a 4-hydroxybutanoic acid (4-HB) and/or 1,4-butanediol (1,4-BDO) biosynthetic pathway comprising an α-ketoglutarate decarboxylase, or an α-ketoglutarate dehydrogenase and a CoA-dependent succinic semialdehyde dehydrogenase, or a glutamate:succinate semialdehyde transaminase and a glutamate decarboxylase; a 4-hydroxybutanoate dehydrogenase; a 4-hydroxybutyryl-CoA:acetyl-CoA transferase, or a butyrate kinase and a phosphotransbutyrylase; an aldehyde dehydrogenase; and an alcohol dehydrogenase.

In another embodiment, the microbial organism can comprise a 4-HB and/or BDO pathway comprising alpha-ketoglutarate decarboxylase, or alpha-ketoglutarate dehydrogenase and CoA-dependent succinate semialdehyde dehydrogenase, or glutamate:succinate semialdehyde transaminase and glutamate decarboxylase; 4-hydroxybutyrate dehydrogenase; 4-hydroxybutyryl-CoA transferase, or 4-hydroxybutyrate kinase and phosphotrans-4-hydroxybutyrylase; 4-hydroxybutyryl-CoA reductase; and 4-hydroxybutyraldehyde reductase, or aldehyde/alcohol dehydrogenase. In still another embodiment, the microbial organisms can comprise a 4-HB and/or BDO pathway comprising alpha-ketoglutarate decarboxylase, or succinyl-CoA synthetase and CoA-dependent succinate semialdehyde dehydrogenase; 4-hydroxybutyrate dehydrogenase; 4-hydroxybutyryl-CoA transferase, or 4-hydroxybutyrate kinase and phosphotrans-4-hydroxybutyrylase; and aldehyde dehydrogenase; and alcohol dehydrogenase, or aldehyde/alcohol dehydrogenase. In another embodiment, the microbial organism can comprise a 4-HB and/or BDO pathway comprising alpha-ketoglutarate decarboxylase, or glutamate dehydrogenase and glutamate decarboxylase, and deaminating 4-aminobutyrate oxidoreductase or 4-aminobutyrate transaminase, or alpha-ketoglutarate dehydrogenase and CoA-dependent succinate semialdehyde dehydrogenase; 4-hydroxybutyrate dehydrogenase; and 4-hydroxybutyrate kinase and phosphotrans-4-hydroxybutyrylase and 4-hydroxybutyryl-CoA reductase and 4-hydroxybutyraldehyde reductase, or 4-hydroxybutyrate kinase and phosphorylating 4-hydroxybutanal dehydrogenase and 4-hydroxybutyraldehyde reductase, or 4-hydroxybutyrate kinase and phosphotrans-4-hydroxybutyrylase and alcohol forming 4-hydroxybutyryl-CoA reductase, or 4-hydroxybutyryl-CoA transferase or 4-hydroxybutyryl-CoA hydrolase or 4-hydroxybutyryl-CoA ligase and 4-hydroxybutyryl-CoA reductase and 4-hydroxybutyraldehyde reductase, or 4-hydroxybutyryl-CoA transferase or 4-hydroxybutyryl-CoA hydrolase or 4-hydroxybutyryl-CoA ligase and alcohol forming 4-hydroxybutyryl-CoA reductase. In yet another embodiment, the microbial organism can comprise a BDO pathway comprising glutamate CoA transferase or glutamyl-CoA hydrolase or glutamyl-CoA ligase and glutamyl-CoA reductase and glutamate-5-semialdehyde reductase, or glutamate CoA transferase or glutamyl-CoA hydrolase or glutamyl-CoA ligase and alcohol forming glutamyl-CoA reductase, or glutamate 5-kinase and phosphorylating glutamate-5-semialdehyde dehydrogenase and glutamate-5-semialdehyde reductase; deaminating 2-amino-5-hydroxypentanoic acid oxidoreductase or 2-amino-5-hydroxypentanoic acid transaminase; and 5-hydroxy-2-oxopentanoic acid decarboxylase and 4-hydroxybutyraldehyde reductase, or decarboxylating 5-hydroxy-2-oxopentanoic acid dehydrogenase and 4-hydroxybutyryl-CoA reductase and 4-hydroxybutyraldehyde reductase; or decarboxylating 5-hydroxy-2-oxopentanoic acid dehydrogenase and alcohol forming 4-hydroxybutyryl-CoA reductase.

In a further embodiment of the invention, the microbial organism can comprise a 4-HB and/or BDO pathway comprising 3-hydroxybutyryl-CoA dehydrogenase; 3-hydroxybutyryl-CoA dehydratase; vinylacetyl-CoA Δ-isomerase; 4-hydroxybutyryl-CoAdehydratase; and 4-hydroxybutyryl-CoA reductase (alcohol forming); or 4-hydroxybutyryl-CoA reductase and 1,4-butanediol dehydrogenase.

In still a further embodiment of the invention, the microbial organism can comprise a 4-HB and/or BDO pathway comprising glutamate CoA transferase; glutamyl-CoA hydrolase; glutamyl-CoA ligase; glutamate 5-kinase; glutamate-5-semialdehyde dehydrogenase (phosphorylating); glutamyl-CoA reductase; glutamate-5-semialdehyde reductase; glutamyl-CoA reductase (alcohol forming); 2-amino-5-hydroxypentanoic acid oxidoreductase (deaminating); 2-amino-5-hydroxypentanoic acid transaminase; 5-hydroxy-2-oxopentanoic acid decarboxylase; and 5-hydroxy-2-oxopentanoic acid dehydrogenase (decarboxylation); and optionally 4-hydroxybutyryl-CoA reductase (alcohol forming), 4-hydroxybutyryl-CoA reductase, or 1,4-butanediol dehydrogenase. In another embodiment, the microbial organism can comprise a 4-HB and/or BDO pathway comprising glutamate CoA transferase; glutamyl-CoA hydrolase; glutamyl-CoA ligase; glutamate 5-kinase; glutamate-5-semialdehyde dehydrogenase (phosphorylating); glutamyl-CoA reductase; glutamate-5-semialdehyde reductase; glutamyl-CoA reductase (alcohol forming); 2-amino-5-hydroxypentanoic acid oxidoreductase (deaminating); 2-amino-5-hydroxypentanoic acid transaminase; 5-hydroxy-2-oxopentanoic acid decarboxylase; and 5-hydroxy-2-oxopentanoic acid dehydrogenase (decarboxylation); and optionally 4-hydroxybutyryl-CoA reductase (alcohol forming), 4-hydroxybutyryl-CoA reductase, or 1,4-butanediol dehydrogenase.

In another embodiment of the invention, the microbial organism can comprise a 4-HB and/or BDO pathway comprising glutamate dehydrogenase; glutamate decarboxylase; 4-aminobutyrate oxidoreductase (deaminating); and 4-hydroxybutyrate dehydrogenase; and further comprising (a) 4-hydroxybutyrate kinase; phosphotrans-4-hydroxybutyrylase; 4-hydroxybutyryl-CoA reductase; and 1,4-butanediol dehydrogenase; (b) 4-hydroxybutyryl-CoA transferase, 4-hydroxybutyryl-CoA hydrolase, or 4-hydroxybutyryl-CoA ligase; 4-hydroxybutyryl-CoA reductase; and 1,4-butanediol dehydrogenase; (c) 4-hydroxybutyryl-CoA transferase, 4-hydroxybutyryl-CoA hydrolase, or 4-hydroxybutyryl-CoA ligase; and 4-hydroxybutyryl-CoA reductase (alcohol forming); (d) 4-hydroxybutyrate kinase; 4-hydroxybutanal dehydrogenase (phosphorylating); and 1,4-butanediol; or (e) 4-hydroxybutyrate kinase; phosphotrans-4-hydroxybutyrylase; and 4-hydroxybutyryl-CoA reductase (alcohol forming). In still another embodiment, the microbial organism can comprise a 4-HB and/or BDO pathway comprising alpha-ketoglutarate decarboxylase, or glutamate dehydrogenase and glutamate decarboxylase and 4-aminobutyrate transaminase; and 4-hydroxybutyrate dehydrogenase; and further comprising (a) 4-hydroxybutyryl-CoA ligase; 4-hydroxybutyryl-CoA reductase; and 1,4-butanediol dehydrogenase; (b) 4-hydroxybutyryl-CoA transferase, 4-hydroxybutyryl-CoA hydrolase, or 4-hydroxybutyryl-CoA ligase; and 4-hydroxybutyryl-CoA reductase (alcohol forming); (c) 4-hydroxybutyrate kinase; 4-hydroxybutanal dehydrogenase (phosphorylating); and 1,4-butanediol dehydrogenase; or (d) 4-hydroxybutyrate kinase; phosphotrans-4-hydroxybutyrylase; and 4-hydroxybutyryl-CoA reductase (alcohol forming). In yet another embodiment, the microbial organism can comprise a 4-HB and/or BDO pathway comprising alpha-ketoglutarate dehydrogenase; CoA-dependent succinic semialdehyde dehydrogenase; and 4-hydroxybutyrate dehydrogenase; and further comprising (a) 4-hydroxybutyryl-CoA ligase; 4-hydroxybutyryl-CoA reductase; and 1,4-butanediol dehydrogenase; (b) 4-hydroxybutyryl-CoA transferase, 4-hydroxybutyryl-CoA hydrolase, or 4-hydroxybutyryl-CoA ligase; and 4-hydroxybutyryl-CoA reductase (alcohol forming); (c) 4-hydroxybutyrate kinase; 4-hydroxybutanal dehydrogenase (phosphorylating); and 1,4-butanediol dehydrogenase; or (d) 4-hydroxybutyrate kinase; phosphotrans-4-hydroxybutyrylase; and 4-hydroxybutyryl-CoA reductase (alcohol forming).

In a further embodiment, the microbial organism can comprise a 4-HB and/or 4-hydroxybutanal and/or BDO pathway comprising succinyl-CoA reductase (aldehyde forming); 4-hydroxybutyrate dehydrogenase; and 4-hydroxybutyrate reductase, also referred to herein as carboxylic acid reductase. In another embodiment, the microbial organism can comprise a 4-HB and/or 4-hydroxybutanal and/or BDO pathway comprising alpha-ketoglutarate decarboxylase; 4-hydroxybutyrate dehydrogenase; and 4-hydroxybutyrate reductase, also referred to herein as carboxylic acid reductase.

For example, the BDO pathway can comprise 4-hydroxybutanoate dehydrogenase, succinyl-CoA synthetase, CoA-dependent succinic semialdehyde dehydrogenase, 4-hydroxybutyrate:CoA transferase, 4-butyrate kinase, phosphotransbutyrylase, alpha-ketoglutarate decarboxylase, aldehyde dehydrogenase, alcohol dehydrogenase or an aldehyde/alcohol dehydrogenase (see FIG. 1). Alternatively, the BDO pathway can comprise 4-aminobutyrate CoA transferase, 4-aminobutyryl-CoA hydrolase, 4-aminobutyrate-CoA ligase, 4-aminobutyryl-CoA oxidoreductase (deaminating), 4-aminobutyryl-CoA transaminase, or 4-hydroxybutyryl-CoA dehydrogenase (see Table 17 in WO2013/184602). Such a BDO pathway can further comprise 4-hydroxybutyryl-CoA reductase (alcohol forming), 4-hydroxybutyryl-CoA reductase, or 1,4-butanediol dehydrogenase.

Additionally, the BDO pathway can comprise 4-aminobutyrate CoA transferase, 4-aminobutyryl-CoA hydrolase, 4-aminobutyrate-CoA ligase, 4-aminobutyryl-CoA reductase (alcohol forming), 4-aminobutyryl-CoA reductase, 4-aminobutan-1-ol dehydrogenase, 4-aminobutan-1-ol oxidoreductase (deaminating) or 4-aminobutan-1-ol transaminase (see Table 18 in WO2013/184602). Also, the BDO pathway can comprise 4-aminobutyrate kinase, 4-aminobutyraldehyde dehydrogenase (phosphorylating), 4-aminobutan-1-ol dehydrogenase, 4-aminobutan-1-ol oxidoreductase (deaminating), 4-aminobutan-1-ol transaminase, [(4-aminobutanolyl)oxy]phosphonic acid oxidoreductase (deaminating), [(4-aminobutanolyl)oxy]phosphonic acid transaminase, 4-hydroxybutyryl-phosphate dehydrogenase, or 4-hydroxybutyraldehyde dehydrogenase (phosphorylating) (see Table 19 in WO2013/184602). Such a pathway can further comprise 1,4-butanediol dehydrogenase.

The BDO pathway can also comprise alpha-ketoglutarate 5-kinase, 2,5-dioxopentanoic semialdehyde dehydrogenase (phosphorylating), 2,5-dioxopentanoic acid reductase, alpha-ketoglutarate CoA transferase, alpha-ketoglutaryl-CoA hydrolase, alpha-ketoglutaryl-CoA ligase, alpha-ketoglutaryl-CoA reductase, 5-hydroxy-2-oxopentanoic acid dehydrogenase, alpha-ketoglutaryl-CoA reductase (alcohol forming), 5-hydroxy-2-oxopentanoic acid decarboxylase, or 5-hydroxy-2-oxopentanoic acid dehydrogenase (decarboxylation)(see Table 20 in WO2013/184602). Such a BDO pathway can further comprise 4-hydroxybutyryl-CoA reductase (alcohol forming), 4-hydroxybutyryl-CoA reductase, or 1,4-butanediol dehydrogenase. Additionally, the BDO pathway can comprise glutamate CoA transferase, glutamyl-CoA hydrolase, glutamyl-CoA ligase, glutamate 5-kinase, glutamate-5-semialdehyde dehydrogenase (phosphorylating), glutamyl-CoA reductase, glutamate-5-semialdehyde reductase, glutamyl-CoA reductase (alcohol forming), 2-amino-5-hydroxypentanoic acid oxidoreductase (deaminating), 2-amino-5-hydroxypentanoic acid transaminase, 5-hydroxy-2-oxopentanoic acid decarboxylase, 5-hydroxy-2-oxopentanoic acid dehydrogenase (decarboxylation)(see Table 21 in WO2013/184602). Such a BDO pathway can further comprise 4-hydroxybutyryl-CoA reductase (alcohol forming), 4-hydroxybutyryl-CoA reductase, or 1,4-butanediol dehydrogenase.

Additionally, the BDO pathway can comprise 3-hydroxybutyryl-CoA dehydrogenase, 3-hydroxybutyryl-CoA dehydratase, vinylacetyl-CoA Δ-isomerase, or 4-hydroxybutyryl-CoA dehydratase (see Table 22 in WO2013/184602). Also, the BDO pathway can comprise homoserine deaminase, homoserine CoA transferase, homoserine-CoA hydrolase, homoserine-CoA ligase, homoserine-CoA deaminase, 4-hydroxybut-2-enoyl-CoA transferase, 4-hydroxybut-2-enoyl-CoA hydrolase, 4-hydroxybut-2-enoyl-CoA ligase, 4-hydroxybut-2-enoate reductase, 4-hydroxybutyryl-CoA transferase, 4-hydroxybutyryl-CoA hydrolase, 4-hydroxybutyryl-CoA ligase, or 4-hydroxybut-2-enoyl-CoA reductase (see Table 23 in WO2013/184602). Such a BDO pathway can further comprise 4-hydroxybutyryl-CoA reductase (alcohol forming), 4-hydroxybutyryl-CoA reductase, or 1,4-butanediol dehydrogenase.

The BDO pathway can additionally comprise succinyl-CoA reductase (alcohol forming), 4-hydroxybutyryl-CoA hydrolase, 4-hydroxybutyryl-CoA ligase, or 4-hydroxybutanal dehydrogenase (phosphorylating)(see Table 15 in WO2013/184602). Such a pathway can further comprise succinyl-CoA reductase, 4-hydroxybutyrate dehydrogenase, 4-hydroxybutyryl-CoA transferase, 4-hydroxybutyrate kinase, phosphotrans-4-hydroxybutyrylase, 4-hydroxybutyryl-CoA reductase, 4-hydroxybutyryl-CoA reductase (alcohol forming), or 1,4-butanediol dehydrogenase. Also, the BDO pathway can comprise glutamate dehydrogenase, 4-aminobutyrate oxidoreductase (deaminating), 4-aminobutyrate transaminase, glutamate decarboxylase, 4-hydroxybutyryl-CoA hydrolase, 4-hydroxybutyryl-CoA ligase, or 4-hydroxybutanal dehydrogenase (phosphorylating)(see Table 16 in WO2013/184602). Such a BDO pathway can further comprise alpha-ketoglutarate decarboxylase, 4-hydroxybutyrate dehydrogenase, 4-hydroxybutyryl-CoA transferase, 4-hydroxybutyrate kinase, phosphotrans-4-hydroxybutyrylase, 4-hydroxybutyryl-CoA reductase, 4-hydroxybutyryl-CoA reductase (alcohol forming), or 1,4-butanediol dehydrogenase.

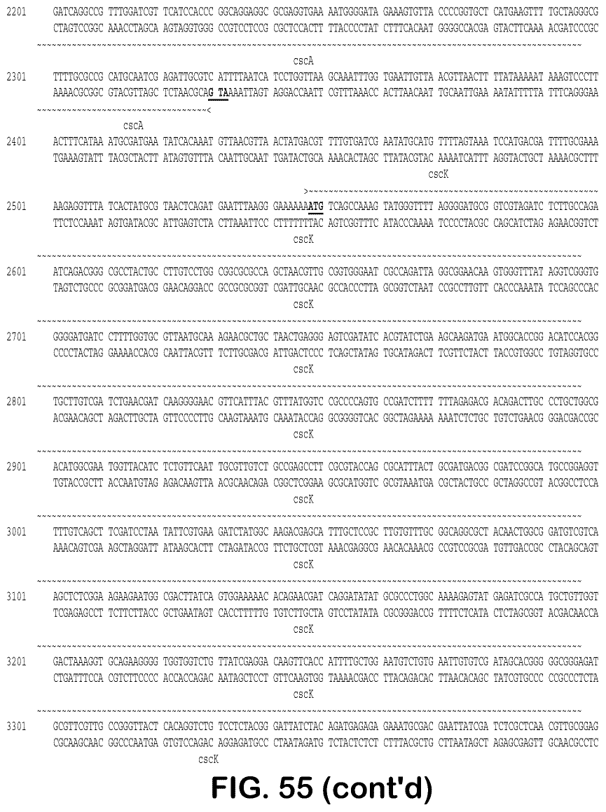

The invention additionally provides a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising succinyl-CoA reductase (aldehyde forming); 4-hydroxybutyrate dehydrogenase; and 4-hydroxybutyrate reductase (see FIG. 58, steps A-C-D). The invention also provides a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising alpha-ketoglutarate decarboxylase; 4-hydroxybutyrate dehydrogenase; and 4-hydroxybutyrate reductase (FIG. 58, steps B-C-D).

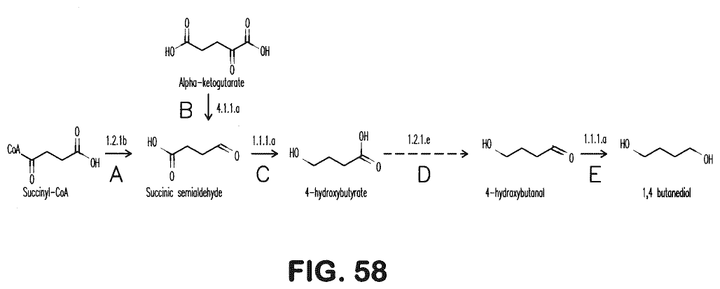

The invention further provides a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising succinate reductase; 4-hydroxybutyrate dehydrogenase, and 4-hydroxybutyrate reductase (see FIG. 62, steps F-C-D). In yet another embodiment, the invention provides a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising alpha-ketoglutarate decarboxylase, or glutamate dehydrogenase or glutamate transaminase and glutamate decarboxylase and 4-aminobutyrate dehydrogenase or 4-aminobutyrate transaminase; 4-hydroxybutyrate dehydrogenase; and 4-hydroxybutyrate reductase (see FIG. 62, steps B or ((J or K)-L-(M or N))-C-D).

The invention also provides a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising alpha-ketoglutarate reductase; 5-hydroxy-2-oxopentanoate dehydrogenase; and 5-hydroxy-2-oxopentanoate decarboxylase (see FIG. 62, steps X-Y-Z). In yet another embodiment, the invention provides a non-naturally occurring microbial organism, comprising a 4-hydroxybutyryl-CoA pathway comprising at least one exogenous nucleic acid encoding a 4-hydroxybutyryl-CoA pathway enzyme expressed in a sufficient amount to produce 4-hydroxybutyryl-CoA, the 4-hydroxybutyryl-CoA pathway comprising alpha-ketoglutarate reductase; 5-hydroxy-2-oxopentanoate dehydrogenase; and 5-hydroxy-2-oxopentanoate dehydrogenase (decarboxylation)(see FIG. 62, steps X-Y-AA).

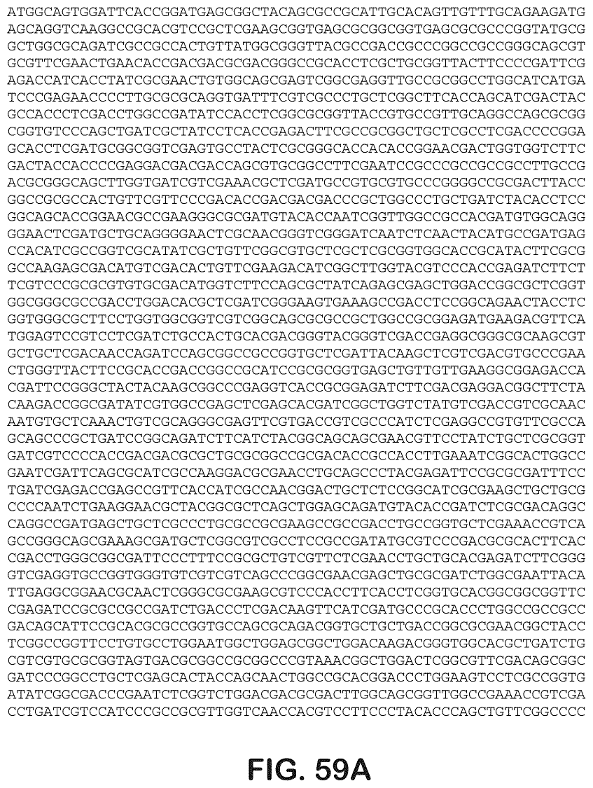

The invention additionally provides a non-naturally occurring microbial organism, comprising a putrescine pathway comprising at least one exogenous nucleic acid encoding a putrescine pathway enzyme expressed in a sufficient amount to produce putrescine, the putrescine pathway comprising succinate reductase; 4-aminobutyrate dehydrogenase or 4-aminobutyrate transaminase; 4-aminobutyrate reductase; and putrescine dehydrogenase or putrescine transaminase (see FIG. 63, steps F-M/N-C-D/E). In still another embodiment, the invention provides a non-naturally occurring microbial organism, comprising a putrescine pathway comprising at least one exogenous nucleic acid encoding a putrescine pathway enzyme expressed in a sufficient amount to produce putrescine, the putrescine pathway comprising alpha-ketoglutarate decarboxylase; 4-aminobutyrate dehydrogenase or 4-aminobutyrate transaminase; 4-aminobutyrate reductase; and putrescine dehydrogenase or putrescine transaminase (see FIG. 63, steps B-M/N-C-D/E). The invention additionally provides a non-naturally occurring microbial organism, comprising a putrescine pathway comprising at least one exogenous nucleic acid encoding a putrescine pathway enzyme expressed in a sufficient amount to produce putrescine, the putrescine pathway comprising glutamate dehydrogenase or glutamate transaminase; glutamate decarboxylase; 4-aminobutyrate reductase; and putrescine dehydrogenase or putrescine transaminase (see FIG. 63, steps J/K-L-C-D/E).

The invention provides in another embodiment a non-naturally occurring microbial organism, comprising a putrescine pathway comprising at least one exogenous nucleic acid encoding a putrescine pathway enzyme expressed in a sufficient amount to produce putrescine, the putrescine pathway comprising alpha-ketoglutarate reductase; 5-amino-2-oxopentanoate dehydrogenase or 5-amino-2-oxopentanoate transaminase; 5-amino-2-oxopentanoate decarboxylase; and putrescine dehydrogenase or putrescine transaminase (see FIG. 63, steps O-P/Q-R-D/E). Also provided is a non-naturally occurring microbial organism, comprising a putrescine pathway comprising at least one exogenous nucleic acid encoding a putrescine pathway enzyme expressed in a sufficient amount to produce putrescine, the putrescine pathway comprising alpha-ketoglutarate reductase; 5-amino-2-oxopentanoate dehydrogenase or 5-amino-2-oxopentanoate transaminase; ornithine dehydrogenase or ornithine transaminase; and ornithine decarboxylase (see FIG. 63, steps O-P/Q-S/T-U).

In an additional embodiment, the invention provides a non-naturally occurring microbial organism having a 4-HB, 4-HBal, 4-HBCoA, BDO or putrescine pathway, wherein the non-naturally occurring microbial organism comprises at least one exogenous nucleic acid encoding an enzyme or protein that converts a substrate of any of the pathways disclosed herein (see, for example, the Examples and FIGS. 1, 8-13, 58, 62, 63, 69 and 70). In an exemplary embodiment for producing BDO, the microbial organism can convert a substrate to a product selected from the group consisting of succinate to succinyl-CoA; succinyl-CoA to succinic semialdehyde; succinic semialdehyde to 4-hydroxybutrate; 4-hydroxybutyrate to 4-hydroxybutyryl-phosphate; 4-hydroxybutyryl-phosphate to 4-hydroxybutyryl-CoA; 4-hydroxybutyryl-CoA to 4-hydroxybutanal; and 4-hydroxybutanal to 1,4-butanediol. In a pathway for producing 4-HBal, a microbial organism can convert, for example, succinate to succinic semialdehyde; succinic semialdehyde to 4-hydroxybutyrate; and 4-hydroxybutyrate to 4-hydroxybutanal. Such an organism can additionally include activity to convert 4-hydroxybutanal to 1,4-butanediol in order to produce BDO. Yet another pathway for producing 4-HBal can be, for example, alpha-ketoglutarate to succinic semialdehyde; succinic semialdehyde to 4-hydroxybutyrate; and 4-hydroxybutyrate to 4-hydroxybutanal. An alternative pathway for producing 4-HBal can be, for example, alpha-ketoglutarate to 2,5-dioxopentanoic acid; 2,5-dioxopentanoic acid to 5-hydroxy-2-oxopentanoic acid; and 5-hydroxy-2-oxopentanoic acid to 4-hydroxybutanal. An exemplary 4-hydroxybutyryl-CoA pathway can be, for example, alpha-ketoglutarate to 2,5-dioxopentanoic acid; 2,5-dioxopentanoic acid to 5-hydroxy-2-oxopentanoic acid; and 5-hydroxy-2-oxopentanoic acid to 4-hydroxybutyryl-CoA. An exemplary putrescine pathway can be, for example, succinate to succinyl-CoA; succinyl-CoA to succinic semialdehyde; succinic semialdehyde to 4-aminobutyrate; 4-aminobutyrate to 4-aminobutanal; and 4-aminobutanal to putrescine. An alternative putrescine pathway can be, for example, succinate to succinic semialdehyde; succinic semialdehyde to 4-aminobutyrate; 4-aminobutyrate to 4-aminobutanal; and 4-aminobutanal to putrescine. One skilled in the art will understand that these are merely exemplary and that any of the substrate-product pairs disclosed herein suitable to produce a desired product and for which an appropriate activity is available for the conversion can be readily determined by one skilled in the art based on the teachings herein. Thus, the invention provides a non-naturally occurring microbial organism containing at least one exogenous nucleic acid encoding an enzyme or protein, where the enzyme or protein converts the substrates and products of a pathway (see FIGS. 1, 8-13, 58, 62, 63, 69 and 70).

While generally described herein as a microbial organism that contains a 4-HB, 4-HBal, 4-HBCoA, BDO or putrescine pathway, it is understood that the invention additionally provides a non-naturally occurring microbial organism comprising at least one exogenous nucleic acid encoding a 4-HB, 4-HBal, 4-HBCoA, BDO or putrescine pathway enzyme or protein expressed in a sufficient amount to produce an intermediate of a 4-HB, 4-HBal, 4-HBCoA, BDO or putrescine pathway. For example, as disclosed herein, 4-HB, 4-HBal, 4-HBCoA, BDO and putrescine pathways are exemplified in FIGS. 1, 8-13, 58, 62, 63, 69 and 70. Therefore, in addition to a microbial organism containing, for example, a BDO pathway that produces BDO, the invention additionally provides a non-naturally occurring microbial organism comprising at least one exogenous nucleic acid encoding a BDO pathway enzyme, where the microbial organism produces a BDO pathway intermediate as a product rather than an intermediate of the pathway. In one exemplary embodiment as shown in FIG. 62, for example, the invention provides a microbial organism that produces succinyl-CoA, succinic semialdehyde, 4-hydroxybutyrate, 4-hydroxybutyryl-phosphate, 4-hydroxybutyryl-CoA, or 4-hydroxybutanal as a product rather than an intermediate. Another exemplary embodiment includes, for example, a microbial organism that produces alpha-ketoglutarate, 2,5-dioxopentanoic acid, 5-hydroxy-2-oxopentanoic acid, or 4-hydroxybutanal as a product rather than an intermediate. An exemplary embodiment in a putrescine pathway includes, for example, a microbial organism that produces glutamate, 4-aminobutyrate, or 4-aminobutanal as a product rather than an intermediate. An alternative embodiment in a putrescine pathway can be, for example, a microbial organism that produces 2,5-dioxopentanoate, 5-amino-2-oxopentanoate, or ornithine as a product rather than an intermediate.

It is understood that any of the pathways disclosed herein, as described in the Examples and exemplified in the Figures, including the pathways of FIGS. 1, 8-13, 58, 62, 63, 69 and 70, can be utilized to generate a non-naturally occurring microbial organism that produces any pathway intermediate or product, as desired. As disclosed herein, such a microbial organism that produces an intermediate can be used in combination with another microbial organism expressing downstream pathway enzymes to produce a desired product. However, it is understood that a non-naturally occurring microbial organism that produces a 4-HB, 4-HBal, 4-HBCoA, BDO or putrescine pathway intermediate can be utilized to produce the intermediate as a desired product.

The invention is described herein with general reference to the metabolic reaction, reactant or product thereof, or with specific reference to one or more nucleic acids or genes encoding an enzyme associated with or catalyzing the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction and that reference to any of these metabolic constituents also references the gene or genes encoding the enzymes that catalyze the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes as well as the reactants and products of the reaction.

As disclosed herein, the product 4-hydroxybutyrate, as well as other intermediates, are carboxylic acids, which can occur in various ionized forms, including fully protonated, partially protonated, and fully deprotonated forms. Accordingly, the suffix “-ate,” or the acid form, can be used interchangeably to describe both the free acid form as well as any deprotonated form, in particular since the ionized form is known to depend on the pH in which the compound is found. It is understood that carboxylate products or intermediates includes ester forms of carboxylate products or pathway intermediates, such as O-carboxylate and S-carboxylate esters. O- and S-carboxylates can include lower alkyl, that is C1 to C6, branched or straight chain carboxylates. Some such O- or S-carboxylates include, without limitation, methyl, ethyl, n-propyl, n-butyl, i-propyl, sec-butyl, and tert-butyl, pentyl, hexyl O- or S-carboxylates, any of which can further possess an unsaturation, providing for example, propenyl, butenyl, pentyl, and hexenyl O- or S-carboxylates. O-carboxylates can be the product of a biosynthetic pathway. Exemplary O-carboxylates accessed via biosynthetic pathways can include, without limitation, methyl 4-hydroxybutyrate, ethyl 4-hydroxybutyrate, and n-propyl 4-hydroxybutyrate. Other biosynthetically accessible O-carboxylates can include medium to long chain groups, that is C7-C22, O-carboxylate esters derived from fatty alcohols, such heptyl, octyl, nonyl, decyl, undecyl, lauryl, tridecyl, myristyl, pentadecyl, cetyl, palmitolyl, heptadecyl, stearyl, nonadecyl, arachidyl, heneicosyl, and behenyl alcohols, any one of which can be optionally branched and/or contain unsaturations. O-carboxylate esters can also be accessed via a biochemical or chemical process, such as esterification of a free carboxylic acid product or transesterification of an O- or S-carboxylate. S-carboxylates are exemplified by CoA S-esters, cysteinyl S-esters, alkylthioesters, and various aryl and heteroaryl thioesters.

The production of 4-HB via biosynthetic modes using the microbial organisms of the invention is particularly useful because it can produce monomeric 4-HB. The non-naturally occurring microbial organisms of the invention and their biosynthesis of 4-HB and BDO family compounds also is particularly useful because the 4-HB product can be (1) secreted; (2) can be devoid of any derivatizations such as Coenzyme A; (3) avoids thermodynamic changes during biosynthesis; (4) allows direct biosynthesis of BDO, and (5) allows for the spontaneous chemical conversion of 4-HB to γ-butyrolactone (GBL) in acidic pH medium. This latter characteristic also is particularly useful for efficient chemical synthesis or biosynthesis of BDO family compounds such as 1,4-butanediol and/or tetrahydrofuran (THF), for example.

Microbial organisms generally lack the capacity to synthesize 4-HB and therefore any of the compounds disclosed herein to be within the 1,4-butanediol family of compounds or known by those in the art to be within the 1,4-butanediol family of compounds. Moreover, organisms having all of the requisite metabolic enzymatic capabilities are not known to produce 4-HB from the enzymes described and biochemical pathways exemplified herein. Rather, with the possible exception of a few anaerobic microorganisms described further below, the microorganisms having the enzymatic capability to use 4-HB as a substrate to produce, for example, succinate. In contrast, the non-naturally occurring microbial organisms of the invention can generate 4-HB, 4-HBal, 4-HBCoA, BDO and/or putrescine as a product. As described above, the biosynthesis of 4-HB in its monomeric form is not only particularly useful in chemical synthesis of BDO family of compounds, it also allows for the further biosynthesis of BDO family compounds and avoids altogether chemical synthesis procedures.

The non-naturally occurring microbial organisms of the invention that can produce 4-HB, 4-HBal, 4-HBCoA, BDO and/or putrescine are produced by ensuring that a host microbial organism includes functional capabilities for the complete biochemical synthesis of at least one 4-HB, 4-HBal, 4-HBCoA, BDO and/or putrscine biosynthetic pathway of the invention. Ensuring at least one requisite 4-HB, 4-HBal, 4-HBCoA or BDO biosynthetic pathway confers 4-HB biosynthesis capability onto the host microbial organism.

Several 4-HB biosynthetic pathways are exemplified herein and shown for purposes of illustration in FIG. 1. Additional 4-HB and BDO pathways are described in FIGS. 8-13. One 4-HB biosynthetic pathway includes the biosynthesis of 4-HB from succinate (the succinate pathway). The enzymes participating in this 4-HB pathway include CoA-independent succinic semialdehyde dehydrogenase and 4-hydroxybutanoate dehydrogenase. In this pathway, CoA-independent succinic semialdehyde dehydrogenase catalyzes the reverse reaction to the arrow shown in FIG. 1. Another 4-HB biosynthetic pathway includes the biosynthesis from succinate through succinyl-CoA (the succinyl-CoA pathway). The enzymes participating in this 4-HB pathway include succinyl-CoA synthetase, CoA-dependent succinic semialdehyde dehydrogenase and 4-hydroxybutanoate dehydrogenase. Three other 4-HB biosynthetic pathways include the biosynthesis of 4-HB from α-ketoglutarate (the α-ketoglutarate pathways). Hence, a third 4-HB biosynthetic pathway is the biosynthesis of succinic semialdehyde through glutamate:succinic semialdehyde transaminase, glutamate decarboxylase and 4-hydroxybutanoate dehydrogenase. A fourth 4-HB biosynthetic pathway also includes the biosynthesis of 4-HB from α-ketoglutarate, but utilizes α-ketoglutarate decarboxylase to catalyze succinic semialdehyde synthesis. 4-hydroxybutanoate dehydrogenase catalyzes the conversion of succinic semialdehyde to 4-HB. A fifth 4-HB biosynthetic pathway includes the biosynthesis from α-ketoglutarate through succinyl-CoA and utilizes α-ketoglutarate dehydrogenase to produce succinyl-CoA, which funnels into the succinyl-CoA pathway described above. Each of these 4-HB biosynthetic pathways, their substrates, reactants and products are described further below in the Examples. As described herein, 4-HB can further be biosynthetically converted to BDO by inclusion of appropriate enzymes to produce BDO (see Example). Thus, it is understood that a 4-HB pathway can be used with enzymes for converting 4-HB to BDO to generate a BDO pathway.

The non-naturally occurring microbial organisms of the invention can be produced by introducing expressible nucleic acids encoding one or more of the enzymes participating in one or more 4-HB, 4-HBal, 4-HBCoA, BDO or putrescine biosynthetic pathways. Depending on the host microbial organism chosen for biosynthesis, nucleic acids for some or all of a particular 4-HB, 4-HBal, 4-HBCoA, BDO or putrescine biosynthetic pathway can be expressed. For example, if a chosen host is deficient in one or more enzymes in a desired biosynthetic pathway, for example, the succinate to 4-HB pathway, then expressible nucleic acids for the deficient enzyme(s), for example, both CoA-independent succinic semialdehyde dehydrogenase and 4-hydroxybutanoate dehydrogenase in this example, are introduced into the host for subsequent exogenous expression. Alternatively, if the chosen host exhibits endogenous expression of some pathway enzymes, but is deficient in others, then an encoding nucleic acid is needed for the deficient enzyme(s) to achieve 4-HB, 4-HBal, 4-HBCoA, BDO or putrescine biosynthesis. For example, if the chosen host exhibits endogenous CoA-independent succinic semialdehyde dehydrogenase, but is deficient in 4-hydroxybutanoate dehydrogenase, then an encoding nucleic acid is needed for this enzyme to achieve 4-HB biosynthesis. Thus, a non-naturally occurring microbial organism of the invention can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as 4-HB, 4-HBal, 4-HBCoA, BDO and/or putrescine.

In like fashion, where 4-HB biosynthesis is selected to occur through the succinate to succinyl-CoA pathway (the succinyl-CoA pathway), encoding nucleic acids for host deficiencies in the enzymes succinyl-CoA synthetase, CoA-dependent succinic semialdehyde dehydrogenase and/or 4-hydroxybutanoate dehydrogenase are to be exogenously expressed in the recipient host. Selection of 4-B biosynthesis through the α-ketoglutarate to succinic semialdehyde pathway (the α-ketoglutarate pathway) can utilize exogenous expression for host deficiencies in one or more of the enzymes for glutamate:succinic semialdehyde transaminase, glutamate decarboxylase and/or 4-hydroxybutanoate dehydrogenase, or α-ketoglutarate decarboxylase and 4-hydroxybutanoate dehydrogenase. One skilled in the art can readily determine pathway enzymes for production of 4-HB or BDO, as disclosed herein.

Depending on the 4-HB, 4-HBal, 4-HBCoA, BDO or putrescine biosynthetic pathway constituents of a selected host microbial organism, the non-naturally occurring microbial organisms of the invention will include at least one exogenously expressed 4-HB, 4-HB, 4-HBCoA, BDO or putrescine pathway-encoding nucleic acid and up to all encoding nucleic acids for one or more 4-HB or BDO biosynthetic pathways. For example, 4-HB, 4-HBal, 4-HBCoA, BDO or putrescine biosynthesis can be established in a host deficient in a pathway enzyme or protein through exogenous expression of the corresponding encoding nucleic acid. In a host deficient in all enzymes or proteins of a 4-B, 4-HB, 4-HBCoA, BDO or putrescine pathway, exogenous expression of all enzyme or proteins in the pathway can be included, although it is understood that all enzymes or proteins of a pathway can be expressed even if the host contains at least one of the pathway enzymes or proteins. If desired, exogenous expression of all enzymes or proteins in a pathway for production of 4-HB, 4-HB, 4-BCoA, BDO or putrescine can be included. For example, 4-HB biosynthesis can be established from all five pathways in a host deficient in 4-hydroxybutanoate dehydrogenase through exogenous expression of a 4-hydroxybutanoate dehydrogenase encoding nucleic acid. In contrast, 4-HB biosynthesis can be established from all five pathways in a host deficient in all eight enzymes through exogenous expression of all eight of CoA-independent succinic semialdehyde dehydrogenase, succinyl-CoA synthetase, CoA-dependent succinic semialdehyde dehydrogenase, glutamate:succinic semialdehyde transaminase, glutamate decarboxylase, α-ketoglutarate decarboxylase, α-ketoglutarate dehydrogenase and 4-hydroxybutanoate dehydrogenase.

Given the teachings and guidance provided herein, those skilled in the art will understand that the number of encoding nucleic acids to introduce in an expressible form will, at least, parallel 4-HB, 4-HBal, 4-HBCoA, BDO or putrescine pathway deficiencies of the selected host microbial organism. Therefore, a non-naturally occurring microbial organism of the invention can have one, two, three, four, five, six, seven, eight or up to all nucleic acids encoding the enzymes disclosed herein constituting one or more 4-HB, 4-HBal, 4-HBCoA, BDO or putrescine biosynthetic pathways. In some embodiments, the non-naturally occurring microbial organisms also can include other genetic modifications that facilitate or optimize 4-HB, 4-HBal, 4-HBCoA, BDO or putrescine biosynthesis or that confer other useful functions onto the host microbial organism. One such other functionality can include, for example, augmentation of the synthesis of one or more of 4-HB pathway precursors such as succinate, succinyl-CoA, α-ketoglutarate, 4-aminobutyrate, glutamate, acetoacetyl-CoA, and/or homoserine.

Generally, a host microbial organism is selected such that it produces the precursor of a 4-HB, 4-HBal, 4-HBCoA, BDO or putrescine pathway, either as a naturally produced molecule or as an engineered product that either provides de novo production of a desired precursor or increased production of a precursor naturally produced by the host microbial organism. For example, succinyl-CoA, α-ketoglutarate, 4-aminobutyrate, glutamate, acetoacetyl-CoA, and homoserine are produced naturally in a host organism such as E. coli. A host organism can be engineered to increase production of a precursor, as disclosed herein. In addition, a microbial organism that has been engineered to produce a desired precursor can be used as a host organism and further engineered to express enzymes or proteins of a 4-B, 4-HBal, 4-HBCoA, BDO or putrescine pathway.

In some embodiments, a non-naturally occurring microbial organism of the invention is generated from a host that contains the enzymatic capability to synthesize 4-HB, 4-HBal, 4-HBCoA, BDO or putrescine. In this specific embodiment it can be useful to increase the synthesis or accumulation of a 4-B, 4-HBal, 4-HBCoA, BDO or putrescine pathway product to, for example, drive 4-HB, 4-HBal, 4-HBCoA, BDO or putrescine pathway reactions toward 4-HB, 4-HBal, 4-HBCoA, BDO or putrescine production. Increased synthesis or accumulation can be accomplished by, for example, overexpression of nucleic acids encoding one or more of the 4-HB, 4-HBal, 4-HBCoA, BDO or putrescine pathway enzymes disclosed herein. Over expression of the 4-HB, 4-HBal, 4-HBCoA, BDO or putrescine pathway enzyme or enzymes can occur, for example, through exogenous expression of the endogenous gene or genes, or through exogenous expression of the heterologous gene or genes. Therefore, naturally occurring organisms can be readily generated to be non-naturally 4-HB, 4-HBal, 4-HBCoA, BDO or putrescine producing microbial organisms of the invention through overexpression of one, two, three, four, five, six and so forth up to all nucleic acids encoding 4-HB, 4-HBal, 4-HBCoA, BDO or putrescine biosynthetic pathway enzymes. In addition, a non-naturally occurring organism can be generated by mutagenesis of an endogenous gene that results in an increase in activity of an enzyme in the 4-HB, 4-HBal, 4-HBCoA, BDO or putrescine biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism (see Examples).

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is further understood, as disclosed herein, that such more than one exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

As used herein, the term “gene disruption,” or grammatical equivalents thereof, is intended to mean a genetic alteration that renders the encoded gene product inactive or attenuated. The genetic alteration can be, for example, deletion of the entire gene, deletion of a regulatory sequence required for transcription or translation, deletion of a portion of the gene which results in a truncated gene product, or by any of various mutation strategies that inactivate or attenuate the encoded gene product. One particularly useful method of gene disruption is complete gene deletion because it reduces or eliminates the occurrence of genetic reversions in the non-naturally occurring microorganisms of the invention. Agene disruption also includes a null mutation, which refers to a mutation within a gene or a region containing a gene that results in the gene not being transcribed into RNA and/or translated into a functional gene product. Such a null mutation can arise from many types of mutations including, for example, inactivating point mutations, deletion of a portion of a gene, entire gene deletions, or deletion of chromosomal segments.

As used herein, the term “attenuate,” or grammatical equivalents thereof, is intended to mean to weaken, reduce or diminish the activity or amount of an enzyme or protein. Attenuation of the activity or amount of an enzyme or protein can mimic complete disruption if the attenuation causes the activity or amount to fall below a critical level required for a given pathway to function. However, the attenuation of the activity or amount of an enzyme or protein that mimics complete disruption for one pathway, can still be sufficient for a separate pathway to continue to function. For example, attenuation of an endogenous enzyme or protein can be sufficient to mimic the complete disruption of the same enzyme or protein for production of a 4-HB, 4-HBal, 4-HBCoA, BDO or putrescine of the invention, but the remaining activity or amount of enzyme or protein can still be sufficient to maintain other pathways, such as a pathway that is critical for the host microbial organism to survive, reproduce or grow. Attenuation of an enzyme or protein can also be weakening reducing or diminishing the activity or amount of the enzyme or protein in an amount that is sufficient to increase yield of a 4-HB, 4-HBal, 4-HBCoA, BDO or putrescine of the invention, but does not necessarily mimic complete disruption of the enzyme or protein.

As used herein, the term “growth-coupled” when used in reference to the production of a biochemical product is intended to mean that the biosynthesis of the referenced biochemical product is produced during the growth phase of a microorganism. In a particular embodiment, the growth-coupled production can optionally be obligatory, meaning that the biosynthesis of the referenced biochemical is an obligatory product produced during the growth phase of a microorganism.

Sources of encoding nucleic acids for a 4-HB, 4-HBal, 4-HBCoA, BDO or putrescine pathway enzyme can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Saccharomyces cerevisiae, Saccharomyces kluyveri, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandi, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter; Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Khuyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, and others disclosed herein (see Examples). For example, microbial organisms having 4-HB, 4-HBal, 4-HBCoA, BDO or putrescine biosynthetic production are exemplified herein with reference to E. coli and yeast hosts. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite 4-HB, 4-HBal, 4-HBCoA, BDO or putrescine biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of 4-HB, 4-HBal, 4-HBCoA, BDO or putrescine and other compounds of the invention described herein with reference to a particular organism such as E. coli or yeast can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative 4-HB, 4-HBal, BDO or putrescine biosynthetic pathway exists in an unrelated species, 4-HB, 4-HBal, BDO or putrescine biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize 4-HB, such as monomeric 4-HB, 4-HBal, BDO or putrescine.

Host microbial organisms can be selected from, and the non-naturally occurring microbial organisms generated in, for example, bacteria, yeast, fungus or any of a variety of other microorganisms applicable to fermentation processes. Exemplary bacteria include any species selected from the order Enterobacteriales, family Enterobacteriaceae, including the genera Escherichia and Klebsiella; the order Aeromonadales, family Succinivibrionaceae, including the genus Anaerobiospirillum; the order Pasteurellales, family Pasteurellaceae, including the genera Actinobacillus and Mannheimia; the order Rhizobiales, family Bradyrhizobiaceae, including the genus Rhizobium; the order Bacillales, family Bacillaceae, including the genus Bacillus; the order Actinomycetales, families Corynebacteriaceae and Streptomycetaceae, including the genus Corynebacterium and the genus Streptomyces, respectively; order Rhodospirillales, family Acetobacteraceae, including the genus Gluconobacter; the order Sphingomonadales, family Sphingomonadaceae, including the genus Zymomonas; the order Lactobacillales, families Lactobacillaceae and Streptococcaceae, including the genus Lactobacillus and the genus Lactococcus, respectively; the order Clostridiales, family Clostridiaceae, genus Clostridium; and the order Pseudomonadales, family Pseudomonadaceae, including the genus Pseudomonas. Non-limiting species of host bacteria include Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida.

Similarly, exemplary species of yeast or fungi species include any species selected from the order Saccharomycetales, family Saccaromycetaceae, including the genera Saccharomyces, Kluyveromyces and Pichia; the order Saccharomycetales, family Dipodascaceae, including the genus Yarrowia; the order Schizosaccharomycetales, family Schizosaccaromycetaceae, including the genus Schizosaccharomyces; the order Eurotiales, family Trichocomaceae, including the genus Aspergillus; and the order Mucorales, family Mucoraceae, including the genus Rhizopus. Non-limiting species of host yeast or fungi include Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. E. coli is a particularly useful host organism since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Methods for constructing and testing the expression levels of a non-naturally occurring 4-HB-, 4-HBal-, 4-HBCoA-, BDO-, or putrescine-producing host can be performed, for example, by recombinant and detection methods well known in the art. Such methods can be found described in, for example, Sambrook et al., Molecular Cloning: A Laboratory Manual, Third Ed, Cold Spring Harbor Laboratory, New York (2001); Ausubel et al., Current Protocols in Molecular Biology, John Wiley and Sons, Baltimore, MD (1999). 4-HB and GBL can be separated by, for example, HPLC using a Spherisorb 5 ODS1 column and a mobile phase of 70% 10 mM phosphate buffer (pH=7) and 30% methanol, and detected using a UV detector at 215 nm (Hennessy et al. 2004, J. Forensic Sci. 46(6):1-9). BDO is detected by gas chromatography or by HPLC and refractive index detector using an Aminex HPX-87H column and a mobile phase of 0.5 mM sulfuric acid (Gonzalez-Pajuelo et al., Met. Eng. 7:329-336 (2005)).

Exogenous nucleic acid sequences involved in a pathway for production of 4-HB, 4-HBal, 4-HBCoA, BDO or putrescine can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to harbor one or more 4-HB, 4-HBal, 4-HBCoA, BDO or putrescine biosynthetic pathway and/or one or more biosynthetic encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The non-naturally occurring microbial organisms of the invention are constructed using methods well known in the art as exemplified herein to exogenously express at least one nucleic acid encoding a 4-HB, 4-HBal, 4-HBCoA, BDO or putrescine pathway enzyme in sufficient amounts to produce 4-HB, such as monomeric 4-HB, 4-HBal, 4-HBCoA, BDO or putrescine. It is understood that the microbial organisms of the invention are cultured under conditions sufficient to produce 4-HB, 4-HBal, 4-HBCoA, BDO or putrescine. Exemplary levels of expression for 4-HB, 4-HBal, 4-HBCoA, BDO or putrescine enzymes in each pathway are described further below in the Examples. Following the teachings and guidance provided herein, the non-naturally occurring microbial organisms of the invention can achieve biosynthesis of 4-HB, such as monomeric 4-HB, 4-HBal, 4-HBCoA, BDO or putrescine resulting in intracellular concentrations between about 0.1-200 mM or more, for example, 0.1-25 mM or more. Generally, the intracellular concentration of 4-HB, such as monomeric 4-HB, 4-HBal, 4-HBCoA, BDO or putrescine is between about 3-150 mM or more, particularly about 5-125 mM or more, and more particularly between about 8-100 mv, for example, about 3-20 mV, particularly between about 5-15 mM and more particularly between about 8-12 mv, including about 10 mM, 20 mM, 50 mM, 80 mM or more. Intracellular concentrations between and above each of these exemplary ranges also can be achieved from the non-naturally occurring microbial organisms of the invention. In particular embodiments, the microbial organisms of the invention, particularly strains such as those disclosed herein (see Examples XII-XIX and Table 18), can provide improved production of a desired product such as 4-HB, 4-HBal, 4-HBCoA, BDO or putrescine by increasing the production of 4-HB, 4-HBal, 4-HBCoA, BDO or putrescine and/or decreasing undesirable byproducts. Such production levels include, but are not limited to, those disclosed herein and including from about 1 gram to about 25 grams per liter, for example about 2, 3, 4, 5, 6, 7, 8, 9, 10, 11, 12, 13, 14, 15, 16, 17, 18, 19, 20, 21, 22, 23, 24, or even higher amounts of product per liter.

In addition to the culturing and fermentation conditions disclosed herein, growth condition for achieving biosynthesis of BDO, 4-HB, 4-HBCoA, 4-HBal and/or putrescine can include the addition of an osmoprotectant to the culturing conditions. In certain embodiments, the non-naturally occurring microbial organisms of the invention can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethylsulfonio-2-methylproprionate, pipecolic acid, dimethylsulfonioacetate, choline, L-camitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mV, no more than about 1.0 mM, no more than about 1.5 mV, no more than about 2.0 mM, no more than about 2.5 mM, no more than about 3.0 mv, no more than about 5.0 mM, no more than about 7.0 mM, no more than about 10 mM, no more than about 50 mM, no more than about 100 mM or no more than about 500 mM.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic conditions or substantially anaerobic, the 4-HB, 4-HBal, 4-HBCoA, BDO or putrescine producers can synthesize 4-HB, 4-HBal, 4-HBCoA, BDO or putrescine at intracellular concentrations of 5-10 mM or more as well as all other concentrations exemplified herein. It is understood that, even though the above description refers to intracellular concentrations, 4-HB, 4-HBal, 4-HBCoA, BDO or putrescine producing microbial organisms can produce 4-HB, 4-HBal, 4-HBCoA, BDO or putrescine intracellularly and/or secrete the product into the culture medium.

Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g, toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

In some embodiments, the carbon feedstock and other cellular uptake sources such as phosphate, ammonia, sulfate, chloride and other halogens can be chosen to alter the isotopic distribution of the atoms present in 4-HB, 4-HBal, 4-HBCoA, BDO or putrescine or any 4-HB, 4-HBal, 4-HBCoA, BDO or putrescine pathway intermediate. The various carbon feedstock and other uptake sources enumerated above will be referred to herein, collectively, as “uptake sources.” Uptake sources can provide isotopic enrichment for any atom present in the product 4-HB, 4-HBal, 4-HBCoA, BDO or putrescine or 4-HB, 4-HBal, 4-HBCoA, BDO or putrescine pathway intermediate including any 4-HB, 4-HBal, 4-HBCoA, BDO or putrescine impurities generated in diverging away from the pathway at any point. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm=(S−B)/(M−B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil. Olsson, The use of Oxalic acid as a Standard. in, Radiocarbon Variations and Absolute Chronology, Nobel Symposium, 12th Proc., John Wiley & Sons, New York (1970). Mass spectrometry results, for example, measured by ASM are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950)¹⁴C/¹²C ratio of 1.176±0.010×10⁻¹² (Karlen et al., Arkiv Geofysik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³.

An oxalic acid standard (SRM 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx His −17.8 per mille. ASTM D6866-11 suggests use of the available Oxalic Acid II standard SRM 4990 C (Hox2) for the modern standard (see discussion of original vs. currently available oxalic acid standards in Mann, Radiocarbon, 25(2):519-527 (1983)). A Fm=00% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well known methods and known standards for determining the biobased content of a compound or material, one skilled in the art can readily determine the biobased content and/or prepared downstream products that utilize of the invention having a desired biobased content.

Applications of carbon-14 dating techniques to quantify bio-based content of materials are known in the art (Currie et al., Nuclear Instruments and Methods in Physics Research B, 172:281-287 (2000)). For example, carbon-14 dating has been used to quantify bio-based content in terephthalate-containing materials (Colonna et al., Green Chemistry, 13:2543-2548 (2011)). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, the present invention provides 4-HB, 4-HBal, 4-HBCoA, BDO or putrescine or a 4-HB, 4-HBal, 4-HBCoA, BDO or putrescine intermediate that has a carbon-12, carbon-13, and carbon-14 ratio that reflects an atmospheric carbon uptake source. For example, in some aspects the 4-HB, 4-HBal, 4-HBCoA, BDO or putrescine or a 4-HB, 4-HBal, 4-HBCoA, BDO or putrescine intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, the present invention provides 4-HB, 4-HBal, 4-HBCoA, BDO or putrescine or a 4-HB, 4-HBal, 4-HBCoA, BDO or putrescine intermediate that has a carbon-12, carbon-13, and carbon-14 ratio that reflects petroleum-based carbon uptake source. In this aspect, the 4-HB, 4-HBal, 4-HBCoA, BDO or putrescine or a 4-HB, 4-HBal, 4-HBCoA, BDO or putrescine intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, the present invention provides 4-HB, 4-HBal, 4-HBCoA, BDO or putrescine or a 4-HB, 4-HBal, 4-HBCoA, BDO or putrescine intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the present invention relates to the biologically produced 4-HB, 4-HBal, 4-HBCoA, BDO or putrescine or 4-HB, 4-Bal, 4-HBCoA, BDO or putrescine intermediate as disclosed herein, and to the products derived therefrom, wherein the 4-HB, 4-HBal, 4-HBCoA, BDO or putrescine or a 4-HB, 4-Bal, 4-HBCoA, BDO or putrescine intermediate has a carbon-12, carbon-13, and carbon-14 isotope ratio of about the same value as the CO₂ that occurs in the environment. For example, in some aspects the invention provides: bioderived 4-HB, 4-HBal, 4-HBCoA, BDO or putrescine or a bioderived 4-HB, 4-HBal, 4-HBCoA, BDO or putrescine intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived 4-HB, 4-HBal, 4-HBCoA, BDO or putrescine or a bioderived 4-HB, 4-HBal, 4-HBCoA, BDO or putrescine intermediate as disclosed herein, wherein the bioderived product is chemically modified to generate a final product. Methods of chemically modifying a bioderived product of 4-HB, 4-HBal, 4-HBCoA, BDO or putrescine, or an intermediate thereof, to generate a desired product are well known to those skilled in the art, as described herein. The invention further provides plastics, elastic fibers, polyurethanes, polyesters, including polyhydroxyalkanoates such as poly-4-hydroxybutyrate (P4HB) or co-polymers thereof, poly(tetramethylene ether) glycol (PTMEG) (also referred to as PTMO, polytetramethylene oxide), polybutylene terephthalate (PBT), and polyurethane-polyurea copolymers, referred to as spandex, elastane or Lycra™, nylons, and the like, having a carbon-12 versus carbon-13 versus carbon-14 isotope ratio of about the same value as the CO₂ that occurs in the environment, wherein the plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide)polybutylene terephthalate (PBT), and polyurethane-polyurea copolymers, referred to as spandex, elastane or Lycra™, and nylons are generated directly from or in combination with bioderived 4-HB, 4-HBal, 4-HBCoA, BDO or putrescine or a bioderived 4-HB, 4-HBal, 4-HBCoA, BDO or putrescine intermediate as disclosed herein.

The products 4-HB, 4-HBal, 4-HBCoA, BDO or putrescine are chemicals commonly used in many commercial and industrial applications. Non-limiting examples of such applications include production of plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, and nylons. Moreover, 4-HB, 4-HBal, 4-HBCoA, BDO or putrescine are also used as a raw material in the production of a wide range of products including plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons. Accordingly, in some embodiments, the invention provides biobased plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons comprising one or more bioderived 4-HB, 4-HBal, 4-HBCoA, BDO or putrescine or bioderived 4-HB, 4-HBal, 4-HBCoA, BDO or putrescine intermediate produced by a non-naturally occurring microorganism of the invention or produced using a method disclosed herein.

As used herein, the term “bioderived” means derived from or synthesized by a biological organism and can be considered a renewable resource since it can be generated by a biological organism. Such a biological organism, in particular the microbial organisms of the invention disclosed herein, can utilize feedstock or biomass, such as, sugars or carbohydrates obtained from an agricultural, plant, bacterial, or animal source. Alternatively, the biological organism can utilize atmospheric carbon. As used herein, the term “biobased” means a product as described above that is composed, in whole or in part, of a bioderived compound of the invention. A biobased or bioderived product is in contrast to a petroleum derived product, wherein such a product is derived from or synthesized from petroleum or a petrochemical feedstock.

In some embodiments, the invention provides plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons comprising bioderived 4-HB, 4-HBal, 4-HBCoA, BDO or putrescine or bioderived 4-HB, 4-HBal, 4-HBCoA, BDO or putrescine intermediate, wherein the bioderived 4-HB, 4-HBal, 4-HBCoA, BDO or putrescine or bioderived 4-HB, 4-HBal, 4-HBCoA, BDO or putrescine intermediate includes all or part of the 4-HB, 4-HBal, 4-HBCoA, BDO or putrescine or 4-HB, 4-HBal, 4-HBCoA, BDO or putrescine intermediate used in the production of plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, and nylons. Thus, in some aspects, the invention provides a biobased plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, and nylons comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived 4-HB, 4-HBal, 4-HBCoA, BDO or putrescine or bioderived 4-HB, 4-HBal, 4-HBCoA, BDO or putrescine intermediate as disclosed herein. Additionally, in some aspects, the invention provides a biobased plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, and nylons wherein the 4-HB, 4-HBal, 4-HBCoA, BDO or putrescine or 4-HB, 4-HBal, 4-HBCoA, BDO or putrescine intermediate used in its production is a combination of bioderived and petroleum derived 4-HB, 4-HBal, 4-HBCoA, BDO or putrescine or 4-HB, 4-HBal, 4-HBCoA, BDO or putrescine intermediate. For example, a biobased plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, and nylons can be produced using 50% bioderived the 4-HB, 4-HBal, 4-HBCoA, BDO or putrescine and 50% petroleum derived 4-HB, 4-HBal, 4-HBCoA, BDO or putrescine or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the microbial organisms disclosed herein. It is understood that methods for producing plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, and nylons using the bioderived 4-HB, 4-HBal, 4-HBCoA, BDO or putrescine or bioderived 4-HB, 4-HBal, 4-HBCoA, BDO or putrescine intermediate of the invention are well known in the art.

The invention additionally provides culture medium, which can be fermentation broth, comprising bioderived 4-hydroxybutyrate or 1,4-butanediol, or other products disclosed herein, for example, 4-HB, 4-HBal, 4-HBCoA, BDO or putrescine, wherein the bioderived product has a carbon-12, carbon-13 and carbon-14 isotope ratio that reflects an atmospheric carbon dioxide uptake source. Such culture medium can comprise bioderived products such as 4-HB, 4-HBal, 4-HBCoA, BDO or putrescine produced by a microbial organism of the invention as disclosed herein. In a particular embodiment, the culture medium can be separated from a non-naturally occurring microbial organism having a 4-HB, 4-HBal, 4-HBCoA, BDO or putrescine pathway, for example, a 4-hydroxybutyrate or 1,4-butanediol pathway. In another embodiment, the invention provides bioderived a 4-HB, 4-HBal, 4-HBCoA, BDO or putrescine, for example, 4-hydroxybutyrate or 1,4-butanediol, having a carbon-12, carbon-13 and carbon-14 isotope ratio that reflects an atmospheric carbon dioxide uptake source, for example, produced by a microbial organism of the invention. In a particular embodiment, the bioderived a 4-HB, 4-HBal, 4-HBCoA, BDO or putrescine, for example, 4-hydroxybutyrate or 1,4-butanediol, can have an Fm value of at least 80%, at least 85%, at least 90%, at least 95% or at least 98%. Such bioderived products of the invention can be produced by the microbial organisms or methods of the invention, as disclosed herein.

The invention further provides a composition comprising bioderived 4-HB, 4-HBal, 4-HBCoA, BDO or putrescine, for example, 4-hydroxybutyrate or 1,4-butanediol, and a compound other than the bioderived 4-HB, 4-HBal, 4-HBCoA, BDO or putrescine, for example, 4-hydroxybutyrate or 1,4-butanediol. The compound other than the bioderived product can be a cellular portion, for example, a trace amount of a cellular portion of, or can be fermentation broth or culture medium or a purified or partially purified fraction thereof produced in the presence of, a non-naturally occurring microbial organism of the invention having a 4-HB, 4-HBal, 4-HBCoA, BDO or putrescine, for example, for example, a 4-hydroxybutyrate or 1,4-butanediol pathway. The composition can comprise, for example, a reduced level of a byproduct when produced by an organism having reduced byproduct formation, as disclosed herein. The composition can comprise, for example, bioderived 4-HB, 4-HBal, 4-HBCoA, BDO or putrescine, for example, 4-hydroxybutyrate or 1,4-butanediol, or a cell lysate or culture supernatant of a microbial organism of the invention.

The invention additionally provides a biobased product comprising biobased 4-HB, 4-HBal, 4-HBCoA, BDO or putrescine, where the biobased product is a plastic, elastic fiber, polyurethane, polyester, polyhydroxyalkanoate, poly-4-hydroxybutyrate (P4HB), or a co-polymer thereof, poly(tetramethylene ether) glycol (PTMEG), polybutylene terephthalate (PBT), polyurethane-polyurea copolymer, or nylon. In one embodiment, the biobased product can comprise at least 5%, at least 10%, at least 20%, at least 30%, at least 40% or at least 50% bioderived 4-HB, 4-HBal, 4-HBCoA, BDO or putrescine, for example, 4-hydroxybutyrate or 1,4-butanediol. In another embodiment, a portion of the biobased product can comprise a portion of the bioderived 4-HB, 4-HBal, 4-HBCoA, BDO or putrescine, for example, 4-hydroxybutyrate or 1,4-butanediol, as a repeating unit, alone or in combination with other monomeric units to form a polymer. In another embodiment, the invention provides a molded product obtained by molding a biobased product such as a plastic, elastic fiber, polyurethane, polyester, polyhydroxyalkanoate, poly-4-hydroxybutyrate (P4HB), or a co-polymer thereof, poly(tetramethylene ether) glycol (PTMEG), polybutylene terephthalate (PBT), polyurethane-polyurea copolymer, or nylon, or other product as disclosed herein. The invention further provides a process for producing a biobased product by chemically reacting bioderived 4-HB, 4-HBal, 4-HBCoA, BDO or putrescine, for example, 4-hydroxybutyrate or 1,4-butanediol, with itself or another compound in a reaction that produces the biobased product. Such bioderived products of the invention can be produced by the microbial organisms or methods of the invention, as disclosed herein.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions.

As described herein, one exemplary growth condition for achieving biosynthesis of 4-HB, 4-HBal, 4-HBCoA, BDO or putrescine includes anaerobic culture or fermentation conditions. In certain embodiments, the non-naturally occurring microbial organisms of the invention can be sustained, cultured or fermented under anaerobic or substantially anaerobic conditions. Briefly, anaerobic conditions refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The invention also provides a non-naturally occurring microbial biocatalyst including a microbial organism having 4-hydroxybutanoic acid (4-HB) and 1,4-butanediol (BDO) biosynthetic pathways that include at least one exogenous nucleic acid encoding 4-hydroxybutanoate dehydrogenase, CoA-independent succinic semialdehyde dehydrogenase, succinyl-CoA synthetase, CoA-dependent succinic semialdehyde dehydrogenase, 4-hydroxybutyrate:CoA transferase, glutamate:succinic semialdehyde transaminase, glutamate decarboxylase, CoA-independent aldehyde dehydrogenase, CoA-dependent aldehyde dehydrogenase or alcohol dehydrogenase, wherein the exogenous nucleic acid is expressed in sufficient amounts to produce 1,4-butanediol (BDO). 4-Hydroxybutyrate:CoA transferase also is known as 4-hydroxybutyryl CoA:acetyl-CoA transferase. Additional 4-HB or BDO pathway enzymes are also disclosed herein (see Examples and FIGS. 8-13).

The invention further provides non-naturally occurring microbial biocatalyst including a microbial organism having 4-hydroxybutanoic acid (4-HB) and 1,4-butanediol (BDO) biosynthetic pathways, the pathways include at least one exogenous nucleic acid encoding 4-hydroxybutanoate dehydrogenase, succinyl-CoA synthetase, CoA-dependent succinic semialdehyde dehydrogenase, 4-hydroxybutyrate:CoA transferase, 4-butyrate kinase, phosphotransbutyrylase, α-ketoglutarate decarboxylase, aldehyde dehydrogenase, alcohol dehydrogenase or an aldehyde/alcohol dehydrogenase, wherein the exogenous nucleic acid is expressed in sufficient amounts to produce 1,4-butanediol (BDO).

Non-naturally occurring microbial organisms also can be generated which biosynthesize BDO. As with the 4-HB producing microbial organisms of the invention, the BDO producing microbial organisms also can produce intracellularly or secret the BDO into the culture medium. Following the teachings and guidance provided previously for the construction of microbial organisms that synthesize 4-HB, additional BDO pathways can be incorporated into the 4-HB producing microbial organisms to generate organisms that also synthesize BDO and other BDO family compounds. The chemical synthesis of BDO and its downstream products are known. The non-naturally occurring microbial organisms of the invention capable of BDO biosynthesis circumvent these chemical synthesis using 4-HB as an entry point as illustrated in FIG. 1. As described further below, the 4-HB producers also can be used to chemically convert 4-B to GBL and then to BDO or THF, for example. Alternatively, the 4-HB producers can be further modified to include biosynthetic capabilities for conversion of 4-HB and/or GBL to BDO.

The additional BDO pathways to introduce into 4-HB producers include, for example, the exogenous expression in a host deficient background or the overexpression of one or more of the enzymes exemplified in FIG. 1 as steps 9-13. One such pathway includes, for example, the enzyme activities necessary to carryout the reactions shown as steps 9, 12 and 13 in FIG. 1, where the aldehyde and alcohol dehydrogenases can be separate enzymes or a multifunctional enzyme having both aldehyde and alcohol dehydrogenase activity. Another such pathway includes, for example, the enzyme activities necessary to carry out the reactions shown as steps 10, 11, 12 and 13 in FIG. 1, also where the aldehyde and alcohol dehydrogenases can be separate enzymes or a multifunctional enzyme having both aldehyde and alcohol dehydrogenase activity. Accordingly, the additional BDO pathways to introduce into 4-HB producers include, for example, the exogenous expression in a host deficient background or the overexpression of one or more of a 4-hydroxybutyrate:CoA transferase, butyrate kinase, phosphotransbutyrylase, CoA-independent aldehyde dehydrogenase, CoA-dependent aldehyde dehydrogenase or an alcohol dehydrogenase. In the absence of endogenous acyl-CoA synthetase capable of modifying 4-HB, the non-naturally occurring BDO producing microbial organisms can further include an exogenous acyl-CoA synthetase selective for 4-HB, or the combination of multiple enzymes that have as a net reaction conversion of 4-HB into 4-HB-CoA. As exemplified further below in the Examples, butyrate kinase and phosphotransbutyrylase exhibit BDO pathway activity and catalyze the conversions illustrated in FIG. 1 with a 4-HB substrate. Therefore, these enzymes also can be referred to herein as 4-hydroxybutyrate kinase and phosphotranshydroxybutyrylase respectively.

Exemplary alcohol and aldehyde dehydrogenases that can be used for these in vivo conversions from 4-HB to BDO are listed below in Table 1.

TABLE 1
Alcohol and Dehydrogenases for Conversion of 4-HB to BDO.
ALCOHOL DEHYDROGENASES

| | |
|---|---|
| ec: 1.1.1.1 | alcohol dehydrogenase |
| ec: 1.1.1.2 | alcohol dehydrogenase (NADP+) |
| ec: 1.1.1.4 | (R,R)-butanediol dehydrogenase |
| ec: 1.1.1.5 | acetoin dehydrogenase |
| ec: 1.1.1.6 | glycerol dehydrogenase |
| ec: 1.1.1.7 | propanediol-phosphate dehydrogenase |
| ec: 1.1.1.8 | glycerol-3-phosphate dehydrogenase |
| | (NAD+) |
| ec: 1.1.1.11 | D-arabinitol 4-dehydrogenase |
| ec: 1.1.1.12 | L-arabinitol 4-dehydrogenase |
| ec: 1.1.1.13 | L-arabinitol 2-dehydrogenase |
| ec: 1.1.1.14 | L-iditol 2-dehydrogenase |
| ec: 1.1.1.15 | D-iditol 2-dehydrogenase |
| ec: 1.1.1.16 | galactitol 2-dehydrogenase |
| ec: 1.1.1.17 | mannitol-1-phosphate 5- |
| | dehydrogenase |
| ec: 1.1.1.18 | inositol 2-dehydrogenase |
| ec: 1.1.1.21 | reductase |
| ec: 1.1.1.23 | histidinol dehydrogenase |
| ec: 1.1.1.26 | glyoxylate reductase |
| ec: 1.1.1.27 | L-lactate dehydrogenase |
| ec: 1.1.1.28 | D-lactate dehydrogenase |
| ec: 1.1.1.29 | glycerate dehydrogenase |
| ec: 1.1.1.30 | 3-hydroxybutyrate dehydrogenase |
| ec: 1.1.1.31 | 3-hydroxyisobutyrate dehydrogenase |
| ec: 1.1.1.35 | 3-hydroxyacyl-CoA dehydrogenase |
| ec: 1.1.1.36 | acetoacetyl-CoA reductase |
| ec: 1.1.1.37 | malate dehydrogenase |
| ec: 1.1.1.38 | malate dehydrogenase (oxaloacetate- |
| | decarboxylating) |
| ec: 1.1.1.39 | malate dehydrogenase |
| | (decarboxylating) |
| ec: 1.1.1.40 | malate dehydrogenase (oxaloacetate- |
| | decarboxylating) (NADP+) |
| ec: 1.1.1.41 | isocitrate dehydrogenase (NAD+) |
| ec: 1.1.1.42 | isocitrate dehydrogenase (NADP+) |
| ec: 1.1.1.54 | allyl-alcohol dehydrogenase |
| ec: 1.1.1.55 | lactreductase (NADPH) |
| ec: 1.1.1.56 | ribitol 2-dehydrogenase |
| ec: 1.1.1.59 | 3-hydroxypropionate dehydrogenase |
| ec: 1.1.1.60 | 2-hydroxy-3-oxopropionate reductase |
| ec: 1.1.1.61 | 4-hydroxybutyrate dehydrogenase |
| ec: 1.1.1.66 | omega-hydroxydecanoate |
| | dehydrogenase |
| ec: 1.1.1.67 | mannitol 2-dehydrogenase |
| ec: 1.1.1.71 | alcohol dehydrogenase [NAD(P)+] |
| ec: 1.1.1.72 | glycerol dehydrogenase (NADP+) |
| ec: 1.1.1.73 | octanol dehydrogenase |
| ec: 1.1.1.75 | (R)-aminopropanol dehydrogenase |
| ec: 1.1.1.76 | (S,S)-butanediol dehydrogenase |
| ec: 1.1.1.77 | lactreductase |
| ec: 1.1.1.78 | methylglyoxal reductase (NADH- |
| | dependent) |
| ec: 1.1.1.79 | glyoxylate reductase (NADP+) |
| ec: 1.1.1.80 | isopropanol dehydrogenase (NADP+) |
| ec: 1.1.1.81 | hydroxypyruvate reductase |
| ec: 1.1.1.82 | malate dehydrogenase (NADP+) |
| ec: 1.1.1.83 | D-malate dehydrogenase |
| | (decarboxylating) |
| ec: 1.1.1.84 | dimethylmalate dehydrogenase |
| ec: 1.1.1.85 | 3-isopropylmalate dehydrogenase |
| ec: 1.1.1.86 | ketol-acid reductoisomerase |
| ec: 1.1.1.87 | homoisocitrate dehydrogenase |
| ec: 1.1.1.88 | hydroxymethylglutaryl-CoA reductase |
| ec: 1.1.1.90 | aryl-alcohol dehydrogenase |
| ec: 1.1.1.91 | aryl-alcohol dehydrogenase (NADP+) |
| ec: 1.1.1.92 | oxaloglycolate reductase |
| | (decarboxylating) |
| ec: 1.1.1.94 | glycerol-3-phosphate dehydrogenase |
| | [NAD(P)+] |
| ec: 1.1.1.95 | phosphoglycerate dehydrogenase |
| ec: 1.1.1.97 | 3-hydroxybenzyl-alcohol |
| | dehydrogenase |
| ec: 1.1.1.101 | acylglycerone-phosphate reductase |
| ec: 1.1.1.103 | L-threonine 3-dehydrogenase |
| ec: 1.1.1.104 | 4-oxoproline reductase |
| ec: 1.1.1.105 | retinol dehydrogenase |
| ec: 1.1.1.110 | indolelactate dehydrogenase |
| ec: 1.1.1.112 | indanol dehydrogenase |
| ec: 1.1.1.113 | L-xylose 1-dehydrogenase |
| ec: 1.1.1.129 | L-threonate 3-dehydrogenase |
| ec: 1.1.1.137 | ribitol-5-phosphate 2-dehydrogenase |
| ec: 1.1.1.138 | mannitol 2-dehydrogenase (NADP+) |
| ec: 1.1.1.140 | sorbitol-6-phosphate 2-dehydrogenase |
| ec: 1.1.1.142 | D-pinitol dehydrogenase |
| ec: 1.1.1.143 | sequoyitol dehydrogenase |
| ec: 1.1.1.144 | perillyl-alcohol dehydrogenase |
| ec: 1.1.1.156 | glycerol 2-dehydrogenase (NADP+) |
| ec: 1.1.1.157 | 3-hydroxybutyryl-CoA dehydrogenase |
| ec: 1.1.1.163 | cyclopentanol dehydrogenase |
| ec: 1.1.1.164 | hexadecanol dehydrogenase |
| ec: 1.1.1.165 | 2-alkyn-1-ol dehydrogenase |
| ec: 1.1.1.166 | hydroxycyclohexanecarboxylate |
| | dehydrogenase |
| ec: 1.1.1.167 | hydroxymalonate dehydrogenase |
| ec: 1.1.1.174 | cyclohexane-1,2-diol dehydrogenase |
| ec: 1.1.1.177 | glycerol-3-phosphate 1- |
| | dehydrogenase (NADP+) |
| ec: 1.1.1.178 | 3-hydroxy-2-methylbutyryl-CoA |
| | dehydrogenase |
| ec: 1.1.1.185 | L-glycol dehydrogenase |
| ec: 1.1.1.190 | indole-3-acetreductase |
| | (NADH) |
| ec: 1.1.1.191 | indole-3-acetreductase |
| | (NADPH) |
| ec: 1.1.1.192 | long-chain-alcohol dehydrogenase |
| ec: 1.1.1.194 | coniferyl-alcohol dehydrogenase |
| ec: 1.1.1.195 | cinnamyl-alcohol dehydrogenase |
| ec: 1.1.1.198 | (+)-borneol dehydrogenase |
| ec: 1.1.1.202 | 1,3-propanediol dehydrogenase |
| ec: 1.1.1.207 | (−)-menthol dehydrogenase |
| ec: 1.1.1.208 | (+)-neomenthol dehydrogenase |
| ec: 1.1.1.216 | farnesol dehydrogenase |
| ec: 1.1.1.217 | benzyl-2-methyl-hydroxybutyrate |
| | dehydrogenase |
| ec: 1.1.1.222 | (R)-4-hydroxyphenyllactate |
| | dehydrogenase |
| ec: 1.1.1.223 | isopiperitenol dehydrogenase |
| ec: 1.1.1.226 | 4-hydroxycyclohexanecarboxylate |
| | dehydrogenase |
| ec: 1.1.1.229 | diethyl 2-methyl-3-oxosuccinate |
| | reductase |
| ec: 1.1.1.237 | hydroxyphenylpyruvate reductase |
| ec: 1.1.1.244 | methanol dehydrogenase |
| ec: 1.1.1.245 | cyclohexanol dehydrogenase |
| ec: 1.1.1.250 | D-arabinitol 2-dehydrogenase |
| ec: 1.1.1.251 | galactitol 1-phosphate 5- |
| | dehydrogenase |
| ec: 1.1.1.255 | mannitol dehydrogenase |
| ec: 1.1.1.256 | fluoren-9-ol dehydrogenase |
| ec: 1.1.1.257 | 4-(hydroxymethyl)benzenesulfonate |
| | dehydrogenase |
| ec: 1.1.1.258 | 6-hydroxyhexanoate dehydrogenase |
| ec: 1.1.1.259 | 3-hydroxypimeloyl-CoA |
| | dehydrogenase |
| ec: 1.1.1.261 | glycerol-1-phosphate dehydrogenase |
| | [NAD(P)+] |
| ec: 1.1.1.265 | 3-methylbutanal reductase |
| ec: 1.1.1.283 | methylglyoxal reductase (NADPH- |
| | dependent) |
| ec: 1.1.1.286 | isocitrate-homoisocitrate |
| | dehydrogenase |
| ec: 1.1.1.287 | D-arabinitol dehydrogenase (NADP+) |
| | butanol dehydrogenase |

ALDEHYDE DEHYDROGENASES

| | |
|---|---|
| ec: 1.2.1.2 | formate dehydrogenase |
| ec: 1.2.1.3 | aldehyde dehydrogenase (NAD+) |
| ec: 1.2.1.4 | aldehyde dehydrogenase (NADP+) |
| ec: 1.2.1.5 | aldehyde dehydrogenase [NAD(P)+] |
| ec: 1.2.1.7 | benzaldehyde dehydrogenase |
| | (NADP+) |
| ec: 1.2.1.8 | betaine-aldehyde dehydrogenase |
| ec: 1.2.1.9 | glyceraldehyde-3-phosphate |
| | dehydrogenase (NADP+) |
| ec: 1.2.1.10 | acetaldehyde dehydrogenase |
| | (acetylating) |
| ec: 1.2.1.11 | aspartate-semialdehyde |
| | dehydrogenase |
| ec: 1.2.1.12 | glyceraldehyde-3-phosphate |
| | dehydrogenase (phosphorylating) |
| ec: 1.2.1.13 | glyceraldehyde-3-phosphate |
| | dehydrogenase (NADP+) (phosphorylating) |
| ec: 1.2.1.15 | malonate-semialdehyde |
| | dehydrogenase |
| ec: 1.2.1.16 | succinate-semialdehyde |
| | dehydrogenase [NAD(P)+] |
| ec: 1.2.1.17 | glyoxylate dehydrogenase (acylating) |
| ec: 1.2.1.18 | malonate-semialdehyde |
| | dehydrogenase (acetylating) |
| ec: 1.2.1.19 | aminobutyraldehyde dehydrogenase |
| ec: 1.2.1.20 | glutarate-semialdehyde |
| | dehydrogenase |
| ec: 1.2.1.21 | glycolaldehyde dehydrogenase |
| ec: 1.2.1.22 | lactaldehyde dehydrogenase |
| ec: 1.2.1.23 | 2-oxoaldehyde dehydrogenase |
| | (NAD+) |
| ec: 1.2.1.24 | succinate-semialdehyde |
| | dehydrogenase |
| ec: 1.2.1.25 | 2-oxoisovalerate dehydrogenase |
| | (acylating) |
| ec: 1.2.1.26 | 2,5-dioxovalerate dehydrogenase |
| ec: 1.2.1.27 | methylmalonate-semialdehyde |
| | dehydrogenase (acylating) |
| ec: 1.2.1.28 | benzaldehyde dehydrogenase (NAD+) |
| ec: 1.2.1.29 | aryl-aldehyde dehydrogenase |
| ec: 1.2.1.30 | aryl-aldehyde dehydrogenase |
| | (NADP+) |
| ec: 1.2.1.31 | L-aminoadipate-semialdehyde |
| | dehydrogenase |
| ec: 1.2.1.32 | aminomuconate-semialdehyde |
| | dehydrogenase |
| ec: 1.2.1.36 | retinal dehydrogenase |
| ec: 1.2.1.39 | phenylacetaldehyde dehydrogenase |
| ec: 1.2.1.41 | glutamate-5-semialdehyde |
| | dehydrogenase |
| ec: 1.2.1.42 | hexadecanal dehydrogenase |
| | (acylating) |
| ec: 1.2.1.43 | formate dehydrogenase (NADP+) |
| ec: 1.2.1.45 | 4-carboxy-2-hydroxymuconate-6- |
| | semialdehyde dehydrogenase |
| ec: 1.2.1.46 | formaldehyde dehydrogenase |
| ec: 1.2.1.47 | 4-trimethylammoniobutyraldehyde |
| | dehydrogenase |
| ec: 1.2.1.48 | long-chain-aldehyde dehydrogenase |
| ec: 1.2.1.49 | 2-oxoaldehyde dehydrogenase |
| | (NADP+) |
| ec: 1.2.1.51 | pyruvate dehydrogenase (NADP+) |
| ec: 1.2.1.52 | oxoglutarate dehydrogenase (NADP+) |
| ec: 1.2.1.53 | 4-hydroxyphenylacetaldehyde |
| | dehydrogenase |
| ec: 1.2.1.57 | butanal dehydrogenase |
| ec: 1.2.1.58 | phenylglyoxylate dehydrogenase |
| | (acylating) |
| ec: 1.2.1.59 | glyceraldehyde-3-phosphate |
| | dehydrogenase (NAD(P)+) (phosphorylating) |
| ec: 1.2.1.62 | 4-formylbenzenesulfonate |
| | dehydrogenase |
| ec: 1.2.1.63 | 6-oxohexanoate dehydrogenase |
| ec: 1.2.1.64 | 4-hydroxybenzaldehyde |
| | dehydrogenase |
| ec: 1.2.1.65 | salicylaldehyde dehydrogenase |
| ec: 1.2.1.66 | mycothiol-dependent formaldehyde |
| | dehydrogenase |
| ec: 1.2.1.67 | vanillin dehydrogenase |
| ec: 1.2.1.68 | coniferyl-aldehyde dehydrogenase |
| ec: 1.2.1.69 | fluoroacetaldehyde dehydrogenase |
| ec: 1.2.1.71 | succinylglutamate-semialdehyde |
| | dehydrogenase |

Other exemplary enzymes and pathways are disclosed herein (see Examples). Furthermore, it is understood that enzymes can be utilized for cany out reactions for which the substrate is not the natural substrate. While the activity for the non-natural substrate may be lower than the natural substrate, it is understood that such enzymes can be utilized, either as naturally occurring or modified using the directed evolution or adaptive evolution, as disclosed herein (see also Examples).

BDO production through any of the pathways disclosed herein are based, in part, on the identification of the appropriate enzymes for conversion of precursors to BDO. A number of specific enzymes for several of the reaction steps have been identified. For those transformations where enzymes specific to the reaction precursors have not been identified, enzyme candidates have been identified that are best suited for catalyzing the reaction steps. Enzymes have been shown to operate on abroad range of substrates, as discussed below. In addition, advances in the field of protein engineering also make it feasible to alter enzymes to act efficiently on substrates, even if not a natural substrate. Described below are several examples of broad-specificity enzymes from diverse classes suitable for a BDO pathway as well as methods that have been used for evolving enzymes to act on non-natural substrates.

A key class of enzymes in BDO pathways is the oxidoreductases that interconvert ketones or aldehydes to alcohols (1.1.1). Numerous exemplary enzymes in this class can operate on a wide range of substrates. An alcohol dehydrogenase (1.1.1.1) purified from the soil bacterium Brevibacterium sp KU 1309 (Hirano et al., J. Biosc. Bioeng. 100:318-322 (2005)) was shown to operate on a plethora of aliphatic as well as aromatic alcohols with high activities. Table 2 shows the activity of the enzyme and its Kₘ on different alcohols. The enzyme is reversible and has very high activity on several aldehydes also (Table 3).

TABLE 2
Relative activities of an alcohol dehydrogenase from
Brevibacterium sp KU to oxidize various alcohols.

| | Relative Activity | Kₘ |
|---|---|---|
| Substrate | (0%) | (mM) |
| 2-Phenylethanol | 100* | 0.025 |
| (S)-2-Phenylpropanol | 156 | 0.157 |
| (R)-2-Phenylpropanol | 63 | 0.020 |
| Bynzyl alcohol | 199 | 0.012 |
| 3-Phenylpropanol | 135 | 0.033 |
| Ethanol | 76 | |
| 1-Butanol | 111 | |
| 1-Octanol | 101 | |
| 1-Dodecanol | 68 | |
| 1-Phenylethanol | 46 | |
| 2-Propanol | 54 | |

*The activity of 2-phenylethanol, corresponding to 19.2 U/mg, was taken as 100%.

TABLE 3
Relative activities of an alcohol dehydrogenase from Brevibacterium
sp KU 1309 to reduce various carbonyl compounds.

| | Relative Activity | Kₘ |
|---|---|---|
| Substrate | (%) | (mM) |
| Phenylacetaldehyde | 100 | 0.261 |
| 2-Phenylpropionaldehyde | 188 | 0.864 |
| 1-Octylaldehyde | 87 | |
| Acetophenone | 0 | |

Lactate dehydrogenase (1.1.1.27) from Ralstonia eutropha is another enzyme that has been demonstrated to have high activities on several 2-oxoacids such as 2-oxobutyrate, 2-oxopentanoate and 2-oxoglutarate (a C5 compound analogous to 2-oxoadipate) (Steinbuchel and Schlegel, Eur. J. Biochem. 130:329-334 (1983)). Column 2 in Table 4 demonstrates the activities of ldhA from R. eutropha (formerly A. eutrophus) on different substrates (Steinbuchel and Schlegel, supra, 1983).

TABLE 4
The in vitro activity of R. eutropha ldhA (Steinbuchel
and Schlegel, supra, 1983) on different
substrates and compared with that on pyruvate.

| | Activity (%) of | | |
|---|---|---|---|
| | L(+)-lactate | L(+)-lactate | D(−)-lactate |
| | dehydrogenase | dehydrogenase | dehydrogenase |
| | from | from | from |
| Substrate | A. eutrophus | rabbit muscle | L. leichmanii |
| Glyoxylate | 8.7 | 23.9 | 5.0 |
| Pyruvate | 100.0 | 100.0 | 100.0 |
| 2-Oxobutyrate | 107.0 | 18.6 | 1.1 |
| 2-Oxovalerate | 125.0 | 0.7 | 0.0 |
| 3-Methyl-2- | 28.5 | 0.0 | 0.0 |
| oxobutyrate | | | |
| 3-Methyl-2- | 5.3 | 0.0 | 0.0 |
| oxovalerate | | | |
| 4-Methyl-2- | 39.0 | 1.4 | 1.1 |
| oxopentanoate | | | |
| Oxaloacetate | 0.0 | 33.1 | 23.1 |
| 2-Oxoglutarate | 79.6 | 0.0 | 0.0 |
| 3-Fluoropyruvate | 33.6 | 74.3 | 40.0 |

Oxidoreductases that can convert 2-oxoacids to their acyl-CoA counterparts (1.2.1) have been shown to accept multiple substrates as well. For example, branched-chain 2-keto-acid dehydrogenase complex (BCKAD), also known as 2-oxoisovalerate dehydrogenase (1.2.1.25), participates in branched-chain amino acid degradation pathways, converting 2-keto acids derivatives of valine, leucine and isoleucine to their acyl-CoA derivatives and CO₂. In some organisms including Rattus norvegicus (Paxton et al., Biochem. J. 234:295-303 (1986)) and Saccharomyces cerevisiae (Sinclair et al., Biochem. Mol Biol. Int. 32:911-922(1993), this complex has been shown to have a broad substrate range that includes linear oxo-acids such as 2-oxobutanoate and alpha-ketoglutarate, in addition to the branched-chain amino acid precursors.

Members of yet another class of enzymes, namely aminotransferases (2.6.1), have been reported to act on multiple substrates. Aspartate aminotransferase (aspAT) from Pyrococcus fursious has been identified, expressed in E. coli and the recombinant protein characterized to demonstrate that the enzyme has the highest activities towards aspartate and alpha-ketoglutarate but lower, yet significant activities towards alanine, glutamate and the aromatic amino acids (Ward et al., Archaea 133-141 (2002)). In another instance, an aminotransferase identified from Leishmania mexicana and expressed in E. coli (Vernal et al., FEMS Microbiol. Lett. 229:217-222(2003)) was reported to have a broad substrate specificity towards tyrosine (activity considered 100% on tyrosine), phenylalanine (90%), tryptophan (85%), aspartate (30%), leucine (25%) and methionine (25%), respectively (Vernal et al., Mol. Biochem. Parasitol 96:83-92(1998)). Similar broad specificity has been reported for a tyrosine aminotransferase from Trypanosoma cruzi, even though both of these enzymes have a sequence homology of only 6%. The latter enzyme can accept leucine, methionine as well as tyrosine, phenylalanine, tryptophan and alanine as efficient amino donors (Nowicki et al., Biochim. Biophys. Acta 1546: 268-281(2001)).

CoA transferases (2.8.3) have been demonstrated to have the ability to act on more than one substrate. Specifically, a CoA transferase was purified from Clostridium acetobutylicum and was reported to have the highest activities on acetate, propionate, and butyrate. It also had significant activities with valerate, isobutyrate, and crotonate (Wiesenborn et al., Appl. Environ. Microbiol. 55:323-329 (1989)). In another study, the E. coli enzyme acyl-CoA:acetate-CoA transferase, also known as acetate-CoA transferase (EC 2.8.3.8), has been shown to transfer the CoA moiety to acetate from a variety of branched and linear acyl-CoA substrates, including isobutyrate (Matthies and Schink, App. Environm. Microbiol. 58:1435-1439 (1992)), valerate (Vanderwinkel et al., Biochem. Biophys. Res Commun. 33:902-908 (1968b)) and butanoate (Vanderwinkel et al., Biochem. Biophys. Res Commun. 33:902-908(1968a).

Other enzyme classes additionally support broad substrate specificity for enzymes. Some isomerases (5.3.3) have also been proven to operate on multiple substrates. For example, L-rhamnose isomerase from Pseudomonas stutzeri catalyzes the isomerization between various aldoalses and ketoses (Yoshida et al., J. Mol. Biol. 365:1505-1516 (2007)). These include isomerization between L-rhamnose and L-rhamnulose, L-mannose and L-fructose, L-xylose and L-xylulose, D-ribose and D-ribulose, and D-allose and D-psicose.

In yet another class of enzymes, the phosphotransferases (2.7.1), the homoserine kinase (2.7.1.39) from E. coli that converts L-homoserine to L-homoserine phosphate, was found to phosphorylate numerous homoserine analogs. In these substrates, the carboxyl functional group at the R-position had been replaced by an ester or by a hydroxymethyl group (Huo and Viola, Biochemistry 35:16180-16185 (1996)). Table 5 demonstrates the broad substrate specificity of this kinase.

TABLE 5
The substrate specificity of homoserine kinase.

| Substrate | k_{cat} | % k_{cat} | Kₘ (mM) | k_{cat}/Kₘ |
|---|---|---|---|---|
| L-homoserine | 18.3 ± 0.1 | 100 | 0.14 ± 0.04 | 184 ± 17 |
| D-homoserine | 8.3 ± 1.1 | 32 | 31.8 ± 7.2 | 0.26 ± 0.03 |
| L-aspartate | 2.1 ± 0.1 | 8.2 | 0.28 ± 0.02 | 7.5 ± 0.3 |
| β-semialdehyde | | | | |
| L-2-amino-1,4- | 2.0 ± 0.5 | 7.9 | 11.6 ± 6.5 | 0.17 ± 0.06 |
| butanediol | | | | |
| L-2-amino-5- | 2.5 ± 0.4 | 9.9 | 1.1 ± 0.5 | 2.3 ± 0.3 |
| hydroxyvalerate | | | | |
| L-homoserine | 14.7 ± 2.6 | 80 | 4.9 ± 2.0 | 3.0 ± 0.6 |
| methyl ester | | | | |
| L-homoserine | 13.6 ± 0.8 | 74 | 1.9 ± 0.5 | 7.2 ± 1.7 |
| ethyl ester | | | | |
| L-homoserine | 13.6 ± 1.4 | 74 | 1.2 ± 0.5 | 11.3 ± 1.1 |
| isopropyl ester | | | | |
| L-homoserine | 14.0 ± 0.4 | 76 | 3.5 ± 0.4 | 4.0 ± 1.2 |
| n-propyl ester | | | | |
| L-homoserine | 16.4 ± 0.8 | 84 | 6.9 ± 1.1 | 2.4 ± 0.3 |
| isobutyl ester | | | | |
| L-homserine | 29.1 ± 1.2 | 160 | 5.8 ± 0.8 | 5.0 ± 0.5 |
| n-butyl ester | | | | |

Another class of enzymes useful in BDO pathways is the acid-thiol ligases (6.2.1). Like enzymes in other classes, certain enzymes in this class have been determined to have broad substrate specificity. For example, acyl CoA ligase from Pseudomonas putida has been demonstrated to work on several aliphatic substrates including acetic, propionic, butyric, valeric, hexanoic, heptanoic, and octanoic acids and on aromatic compounds such as phenylacetic and phenoxyacetic acids (Fernandez-Valverde et al., Appl. Environ. Microbiol. 59:1149-1154 (1993)). A related enzyme, malonyl CoA synthetase (6.3.4.9) from Rhizobium trifolii could convert several diacids, namely, ethyl-, propyl-, allyl-, isopropyl-, dimethyl-, cyclopropyl-, cyclopropylmethylene-, cyclobutyl-, and benzyl-malonate into their corresponding monothioesters (Pohl et al., J. Am. Chem. Soc. 123:5822-5823 (2001)). Similarly, decarboxylases (4.1.1) have also been found with broad substrate ranges. Pyruvate decarboxylase (PDC), also termed keto-acid decarboxylase, is a key enzyme in alcoholic fermentation, catalyzing the decarboxylation of pyruvate to acetaldehyde. The enzyme isolated from Saccharomyces cerevisiae has a broad substrate range for aliphatic 2-keto acids including 2-ketobutyrate, 2-ketovalerate, and 2-phenylpyruvate (Li and Jordan, Biochemistry 38:10004-10012 (1999)). Similarly, benzoylformate decarboxylase has abroad substrate range and has been the target of enzyme engineering studies. The enzyme from Pseudomonas putida has been extensively studied and crystal structures of this enzyme are available (Polovnikova et al., Biochemistry 42:1820-1830 (2003); Hasson et al., Biochemistry 37:9918-9930 (1998)). Branched chain alpha-ketoacid decarboxylase (BCKA) has been shown to act on a variety of compounds varying in chain length from 3 to 6 carbons (Oku and Kaneda, J. Biol. Chem. 263:18386-18396 (1998); Smit et al., Appl. Environ. Microbiol. 71:303-311 (2005b)). The enzyme in Lactococcus lactis has been characterized on a variety of branched and linear substrates including 2-oxobutanoate, 2-oxohexanoate, 2-oxopentanoate, 3-methyl-2-oxobutanoate, 4-methyl-2-oxobutanoate and isocaproate (Smit et al., Appl. Environ. Microbiol. 71:303-311 (2005a).

Interestingly, enzymes known to have one dominant activity have also been reported to catalyze a very different function. For example, the cofactor-dependent phosphoglycerate mutase (5.4.2.1) from Bacillus stearothermophilus and Bacillus subtilis is known to function as a phosphatase as well (Rigden et al., Protein Sci. 10:1835-1846 (2001)). The enzyme from B. stearothermophilus is known to have activity on several substrates, including 3-phosphoglycerate, alpha-napthylphosphate, p-nitrophenylphosphate, AMP, fructose-6-phosphate, ribose-5-phosphate and CMP

In contrast to these examples where the enzymes naturally have broad substrate specificities, numerous enzymes have been modified using directed evolution to broaden their specificity towards their non-natural substrates. Alternatively, the substrate preference of an enzyme has also been changed using directed evolution. Therefore, it is feasible to engineer a given enzyme for efficient function on a natural, for example, improved efficiency, or a non-natural substrate, for example, increased efficiency. For example, it has been reported that the enantioselectivity of a lipase from Pseudomonas aeruginosa was improved significantly (Reetz et al., Agnew. Chem. Int. Ed Engl. 36:2830-2832 (1997)). This enzyme hydrolyzed p-nitrophenyl 2-methyldecanoate with only 2% enantiomeric excess (ee) in favor of the (S)-acid. However, after four successive rounds of error-prone mutagenesis and screening, a variant was produced that catalyzed the requisite reaction with 81% ee (Reetz et al., Agnew. Chem. Int. Ed Engl. 36:2830-2832(1997)).

Directed evolution methods have been used to modify an enzyme to function on an array of non-natural substrates. The substrate specificity of the lipase in P. aeruginosa was broadened by randomization of amino acid residues near the active site. This allowed for the acceptance of alpha-substituted carboxylic acid esters by this enzyme (Reetz et al., Agnew. Chem. Int. Ed Engl. 44:4192-4196 (2005)). In another successful modification of an enzyme, DNA shuffling was employed to create an Escherichia coli aminotransferase that accepted β-branched substrates, which were poorly accepted by the wild-type enzyme (Yano et al., Proc. Nat. Acad. Sci. U.S.A. 95:5511-5515 (1998)). Specifically, at the end of four rounds of shuffling, the activity of aspartate aminotransferase for valine and 2-oxovaline increased by up to five orders of magnitude, while decreasing the activity towards the natural substrate, aspartate, by up to 30-fold. Recently, an algorithm was used to design a retro-aldolase that could be used to catalyze the carbon-carbon bond cleavage in a non-natural and non-biological substrate, 4-hydroxy-4-(6-methoxy-2-naphthyl)-2-butanone (Jiang et al., Science 319:1387-1391 (2008)). These algorithms used different combinations of four different catalytic motifs to design new enzyme, and 20 of the selected designs for experimental characterization had four-fold improved rates over the uncatalyzed reaction (Jiang et al., Science 319:1387-1391(2008)). Thus, not only are these engineering approaches capable of expanding the array of substrates on which an enzyme can act, but they allow the design and construction of very efficient enzymes. For example, a method of DNA shuffling (random chimeragenesis on transient templates or RACHITT) was reported to lead to an engineered monooxygenase that had an improved rate of desulfurization on complex substrates as well as 20-fold faster conversion of a non-natural substrate (Coco et al., Nat. Biotechnol. 19:354-359 (2001)). Similarly, the specific activity of a sluggish mutant triosephosphate isomerase enzyme was improved up to 19-fold from 1.3 fold (Hermes et al., Proc. Nat. Acad Sci. U.S.A. 87:696-700 1990)). This enhancement in specific activity was accomplished by using random mutagenesis over the whole length of the protein and the improvement could be traced back to mutations in six amino acid residues.

The effectiveness of protein engineering approaches to alter the substrate specificity of an enzyme for a desired substrate has also been demonstrated in several studies. Isopropylmalate dehydrogenase from Thermus thermophilus was modified by changing residues close to the active site so that it could now act on malate and D-lactate as substrates (Fujita et al., Biosci. Biotechnol. Biochem. 65:2695-2700 (2001)). In this study as well as in others, it was pointed out that one or a few residues could be modified to alter the substrate specificity. For example, the dihydroflavonol 4-reductase for which a single amino acid was changed in the presumed substrate-binding region could preferentially reduce dihydrokaempferol (Johnson et al., Plant. J. 25:325-333 (2001)). The substrate specificity of a very specific isocitrate dehydrogenase from Escherichia coli was changed form isocitrate to isopropylmalate by changing one residue in the active site (Doyle et al., Biochemistry 40:4234-4241 (2001)). Similarly, the cofactor specificity of a NAD⁺-dependent 1,5-hydroxyprostaglandin dehydrogenase was altered to NADP⁺ by changing a few residues near the N-terminal end (Cho et al., Arch. Biochem. Biophys. 419:139-146 (2003)). Sequence analysis and molecular modeling analysis were used to identify the key residues for modification, which were further studied by site-directed mutagenesis.

Numerous examples exist spanning diverse classes of enzymes where the function of enzyme was changed to favor one non-natural substrate over the natural substrate of the enzyme. A fucosidase was evolved from a galactosidase in E. coli by DNA shuffling and screening (Zhang et al., Proc. Natl Acad. Sci. U.S.A. 94:4504-4509 (1997)). Similarly, aspartate aminotransferase from E. coli was converted into a tyrosine aminotransferase using homology modeling and site-directed mutagenesis (Onuffer and Kirsch, Protein Sci., 4:1750-1757 (1995)). Site-directed mutagenesis of two residues in the active site of benzoylformate decarboxylase from P. putida reportedly altered the affinity (Kₘ) towards natural and non-natural substrates (Siegert et al., Protein Eng Des Sel 18:345-357 (2005)). Cytochrome c peroxidase (CCP) from Saccharomyces cerevisiae was subjected to directed molecular evolution to generate mutants with increased activity against the classical peroxidase substrate guaiacol, thus changing the substrate specificity of CCP from the protein cytochrome c to a small organic molecule. After three rounds of DNA shuffling and screening mutants were isolated which possessed a 300-fold increased activity against guaiacol and up to 1000-fold increased specificity for this substrate relative to that for the natural substrate (Iffland et al., Biochemistry 39:10790-10798 (2000)).

In some cases, enzymes with different substrate preferences than either of the parent enzymes have been obtained. For example, biphenyl-dioxygenase-mediated degradation of polychlorinated biphenyls was improved by shuffling genes from two bacteria, Pseudomonas pseudoalcaligens and Burkholderia cepacia (Kumamaru et al., Nat. Biotechnol. 16:663-666 (1998)). The resulting chimeric biphenyl oxygenases showed different substrate preferences than both the parental enzymes and enhanced the degradation activity towards related biphenyl compounds and single aromatic ring hydrocarbons such as toluene and benzene which were originally poor substrates for the enzyme.

In addition to changing enzyme specificity, it is also possible to enhance the activities on substrates for which the enzymes naturally have low activities. One study demonstrated that amino acid racemase from P. putida that had broad substrate specificity (on lysine, arginine, alanine, serine, methionine, cysteine, leucine and histidine among others) but low activity towards tryptophan could be improved significantly by random mutagenesis (Kino et al., Appl. Microbiol. Biotechnol. 73:1299-1305 (2007)). Similarly, the active site of the bovine BCKAD was engineered to favor alternate substrate acetyl-CoA (Meng and Chuang, Biochemistry 33:12879-12885 (1994)). An interesting aspect of these approaches is that even if random methods have been applied to generate these mutated enzymes with efficacious activities, the exact mutations or structural changes that confer the improvement in activity can be identified. For example, in the aforementioned study, the mutations that facilitated improved activity on tryptophan was traced back to two different positions.

Directed evolution has also been used to express proteins that are difficult to express. For example, by subjecting horseradish peroxidase to random mutagenesis and gene recombination, mutants were identified that had more than 14-fold higher activity than the wild type (Lin et al., Biotechnol. Prog. 15:467471 (1999)).

Another example of directed evolution shows the extensive modifications to which an enzyme can be subjected to achieve a range of desired functions. The enzyme lactate dehydrogenase from Bacillus stearothermophilus was subjected to site-directed mutagenesis, and three amino acid substitutions were made at sites that were believed to determine the specificity towards different hydroxyacids (Clarke et al., Biochem. Biophys. Res. Commun 148:15-23 (1987)). After these mutations, the specificity for oxaloacetate over pyruvate was increased to 500 in contrast to the wild type enzyme that had a catalytic specificity for pyruvate over oxaloacetate of 1000. This enzyme was further engineered using site-directed mutagenesis to have activity towards branched-chain substituted pyruvates (Wilks et al., Biochemistry 29:8587-8591 (1990)). Specifically, the enzyme had a 55-fold improvement in K_{cat} for alpha-ketoisocaproate. Three structural modifications were made in the same enzyme to change its substrate specificity from lactate to malate. The enzyme was highly active and specific towards malate (Wilks et al., Science 242:1541-1544 (1988)). The same enzyme from B. stearothermophilus was subsequently engineered to have high catalytic activity towards alpha-keto acids with positively charged side chains, such as those containing ammonium groups (Hogan et al., Biochemistry 34:42254230 (1995)). Mutants with acidic amino acids introduced at position 102 of the enzyme favored binding of such side chain ammonium groups. The results obtained proved that the mutants showed up to 25-fold improvements in k_{cat}/Kₘ values for omega-amino-alpha-keto acid substrates. Interestingly, this enzyme was also structurally modified to function as a phenyllactate dehydrogenase instead of a lactate dehydrogenase (Wilks et al., Biochemistry 31:7802-7806 1992). Restriction sites were introduced into the gene for the enzyme which allowed a region of the gene to be excised. This region coded for a mobile surface loop of the polypeptide (residues 98-110) which normally seals the active site from bulk solvent and is a major determinant of substrate specificity. The variable length and sequence loops were inserted so that hydroxyacid dehydrogenases with altered substrate specificities were generated. With one longer loop construction, activity with pyruvate was reduced one-million-fold but activity with phenylpyruvate was largely unaltered. A switch in specificity (k_{cat}/Kₘ) of 390,000-fold was achieved. The 1700:1 selectivity of this enzyme for phenylpyruvate over pyruvate is that required in a phenyllactate dehydrogenase. The studies described above indicate that various approaches of enzyme engineering can be used to obtain enzymes for the BDO pathways as disclosed herein.

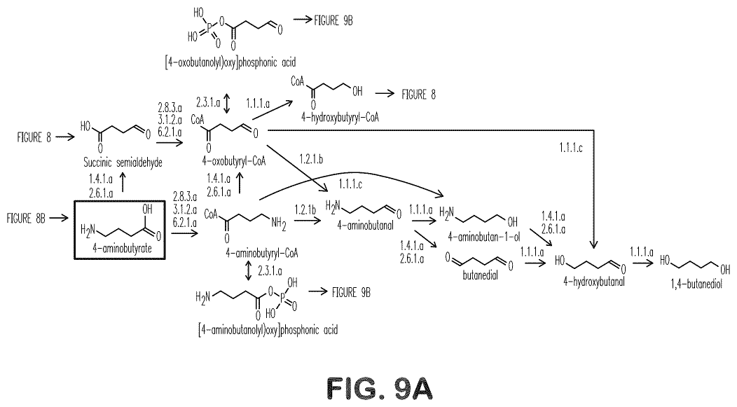

As disclosed herein, biosynthetic pathways to 1,4-butanediol from a number of central metabolic intermediates are can be utilized, including acetyl-CoA, succinyl-CoA, alpha-ketoglutarate, glutamate, 4-aminobutyrate, and homoserine. Acetyl-CoA, succinyl-CoA and alpha-ketoglutarate are common intermediates of the tricarboxylic acid (TCA) cycle, a series of reactions that is present in its entirety in nearly all living cells that utilize oxygen for cellular respiration and is present in truncated forms in a number of anaerobic organisms. Glutamate is an amino acid that is derived from alpha-ketoglutarate via glutamate dehydrogenase or any of a number of transamination reactions (see FIG. 8B). 4-aminobutyrate can be formed by the decarboxylation of glutamate (see FIG. 8B) or from acetoacetyl-CoA via the pathway disclosed in FIG. 9C. Acetoacetyl-CoA is derived from the condensation of two acetyl-CoA molecules by way of the enzyme, acetyl-coenzyme A acetyltransferase, or equivalently, acetoacetyl-coenzyme A thiolase. Homoserine is an intermediate in threonine and methionine metabolism, formed from oxaloacetate via aspartate. The conversion of oxaloacetate to homoserine requires one NADH, two NADPH, and one ATP

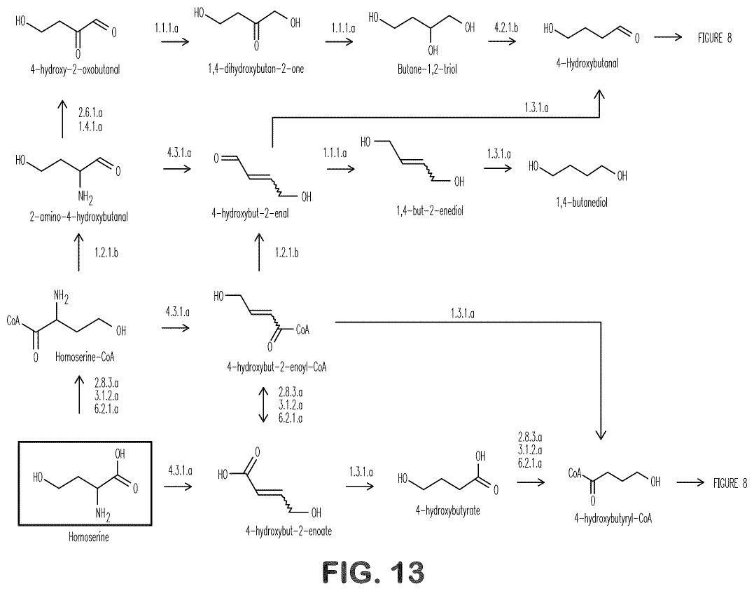

Pathways other than those exemplified above also can be employed to generate the biosynthesis of BDO in non-naturally occurring microbial organisms. In one embodiment, biosynthesis can be achieved using a L-homoserine to BDO pathway (see FIG. 13). This pathway has a molar yield of 0.90 mol/mol glucose, which appears restricted by the availability of reducing equivalents. A second pathway synthesizes BDO from acetoacetyl-CoA and is capable of achieving the maximum theoretical yield of 1.091 mol/mol glucose (see FIG. 9). Implementation of either pathway can be achieved by introduction of two exogenous enzymes into a host organism such as E. coli, and both pathways can additionally complement BDO production via succinyl-CoA. Pathway enzymes, thermodynamics, theoretical yields and overall feasibility are described further below.

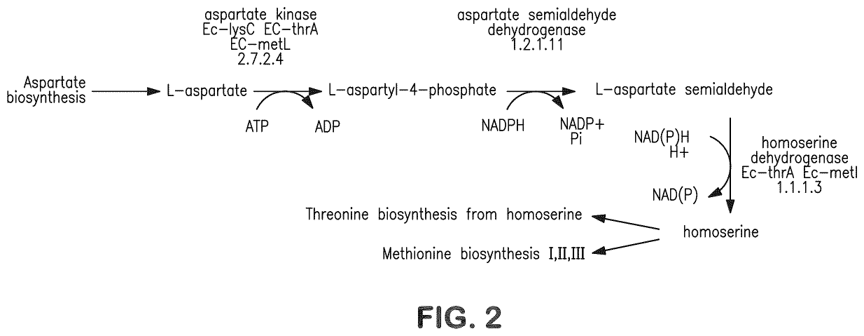

A homoserine pathway also can be engineered to generate BDO-producing microbial organisms. Homoserine is an intermediate in threonine and methionine metabolism, formed from oxaloacetate via aspartate. The conversion of oxaloacetate to homoserine requires one NADH, two NADPH, and one ATP (FIG. 2). Once formed, homoserine feeds into biosynthetic pathways for both threonine and methionine. In most organisms, high levels of threonine or methionine feedback to repress the homoserine biosynthesis pathway (Caspi et al., Nucleic Acids Res. 34:D511-D516 (1990)).

The transformation of homoserine to 4-hydroxybutyrate (4-HB) can be accomplished in two enzymatic steps as described herein. The first step of this pathway is deamination of homoserine by a putative ammonia lyase. In step 2, the product alkene, 4-hydroxybut-2-enoate is reduced to 4-HB by a putative reductase at the cost of one NADH. 4-HB can then be converted to BDO.

Enzymes available for catalyzing the above transformations are disclosed herein. For example, the ammonia lyase in step 1 of the pathway closely resembles the chemistry of aspartate ammonia-lyase (aspartase). Aspartase is a widespread enzyme in microorganisms, and has been characterized extensively (Viola, R. E., Mol. Biol. 74:295-341 (2008)). The crystal structure of the E. coli aspartase has been solved (Shi et al., Biochemistry 36:9136-9144 (1997)), so it is therefore possible to directly engineer mutations in the enzyme's active site that would alter its substrate specificity to include homoserine. The oxidoreductase in step 2 has chemistry similar to several well-characterized enzymes including fumarate reductase in the E. coli TCA cycle. Since the thermodynamics of this reaction are highly favorable, an endogenous reductase with broad substrate specificity will likely be able to reduce 4-hydroxybut-2-enoate. The yield of this pathway under anaerobic conditions is 0.9 mol BDO per mol glucose.

The succinyl-CoA pathway was found to have a higher yield due to the fact that it is more energetically efficient. The conversion of one oxaloacetate molecule to BDO via the homoserine pathway will require the expenditure of 2 ATP equivalents. Because the conversion of glucose to two oxaloacetate molecules can generate a maximum of 3 ATP molecules assuming PEP carboxykinase to be reversible, the overall conversion of glucose to BDO via homoserine has a negative energetic yield. As expected, if it is assumed that energy can be generated via respiration, the maximum yield of the homoserine pathway increases to 1.05 mol/mol glucose which is 960 of the succinyl-CoA pathway yield. The succinyl-CoA pathway can channel some of the carbon flux through pyruvate dehydrogenase and the oxidative branch of the TCA cycle to generate both reducing equivalents and succinyl-CoA without an energetic expenditure. Thus, it does not encounter the same energetic difficulties as the homoserine pathway because not all of the flux is channeled through oxaloacetate to succinyl-CoA to BDO. Overall, the homoserine pathway demonstrates a high-yielding route to BDO.

An acetoacetate pathway also can be engineered to generate BDO-producing microbial organisms. Acetoacetate can be formed from acetyl-CoA by enzymes involved in fatty acid metabolism, including acetyl-CoA acetyltransferase and acetoacetyl-CoA transferase. Biosynthetic routes through acetoacetate are also particularly useful in microbial organisms that can metabolize single carbon compounds such as carbon monoxide, carbon dioxide or methanol to form acetyl-CoA.

A three step route from acetoacetyl-CoA to 4-aminobutyrate (see FIG. 9C) can be used to synthesize BDO through acetoacetyl-CoA. 4-Aminobutyrate can be converted to succinic semialdehyde as shown in FIG. 8B. Succinic semialdehyde, which is one reduction step removed from succinyl-CoA or one decarboxylation step removed from α-ketoglutarate, can be converted to BDO following three reductions steps (FIG. 1). Briefly, step 1 of this pathway involves the conversion of acetoacetyl-CoA to acetoacetate by, for example, the E. coli acetoacetyl-CoA transferase encoded by the atoA and atoD genes (Hanai et al., Appl. Environ. Microbiol. 73: 7814-7818 (2007)). Step 2 of the acetoacetyl-CoA biopathway entails conversion of acetoacetate to 3-aminobutanoate by an ω-aminotransferase. The ω-amino acid:pyruvate aminotransferase (ω-APT) from Alcaligens denitrificans was overexpressed in E. coli and shown to have a high activity toward 3-aminobutanoate in vitro (Yun et al., Appl. Environ. Microbiol. 70:2529-2534 (2004)).

In step 2, a putative aminomutase shifts the amine group from the 3- to the 4-position of the carbon backbone. An aminomutase performing this function on 3-aminobutanoate has not been characterized, but an enzyme from Clostridium sticklandii has a very similar mechanism. The enzyme, D-lysine-5,6-aminomutase, is involved in lysine biosynthesis.

The synthetic route to BDO from acetoacetyl-CoA passes through 4-aminobutanoate, a metabolite in E. coli that is normally formed from decarboxylation of glutamate. Once formed, 4-aminobutanoate can be converted to succinic semialdehyde by 4-aminobutanoate transaminase (2.6.1.19), an enzyme which has been biochemically characterized.

One consideration for selecting candidate enzymes in this pathway is the stereoselectivity of the enzymes involved in steps 2 and 3. The ω-ABT in Alcaligens denitrificans is specific to the L-stereoisomer of 3-aminobutanoate, while D-lysine-5,6-aminomutase likely requires the D-stereoisomer. If enzymes with complementary stereoselectivity are not initially found or engineered, a third enzyme can be added to the pathway with racemase activity that can convert L-3-aminobutanoate to D-3-aminobutanoate. While amino acid racemases are widespread, whether these enzymes can function on ω-amino acids is not known.

The maximum theoretical molar yield of this pathway under anaerobic conditions is 1.091 mol/mol glucose. In order to generate flux from acetoacetyl-CoA to BDO it was necessary to assume that acetyl-CoA:acetoacetyl-CoA transferase is reversible. The function of this enzyme in E. coli is to metabolize short-chain fatty acids by first converting them into thioesters.

While the operation of acetyl-CoA:acetoacetyl-CoA transferase in the acetate-consuming direction has not been demonstrated experimentally in E. coli, studies on similar enzymes in other organisms support the assumption that this reaction is reversible. The enzyme butyryl-CoA:acetate:CoA transferase in gut microbes Roseburia sp. and F. prasnitzii operates in the acetate utilizing direction to produce butyrate (Duncan et al., Appl. Environ. Microbiol. 68:5186-5190 (2002)). Another very similar enzyme, acetyl:succinate CoA-transferase in Trypanosoma brucei, also operates in the acetate utilizing direction. This reaction has a ΔᵣₓₙG close to equilibrium, so high concentrations of acetate can likely drive the reaction in the direction of interest. At the maximum theoretical BDO production rate of 1.09 mol/mol glucose simulations predict that E. coli can generate 1.098 mol ATP per mol glucose with no fermentation byproducts. This ATP yield should be sufficient for cell growth, maintenance, and production. The acetoacetatyl-CoA biopathway is a high-yielding route to BDO from acetyl-CoA.

Therefore, in addition to any of the various modifications exemplified previously for establishing 4-HB biosynthesis in a selected host, the BDO producing microbial organisms can include any of the previous combinations and permutations of 4-HB pathway metabolic modifications as well as any combination of expression for CoA-independent aldehyde dehydrogenase, CoA-dependent aldehyde dehydrogenase or an alcohol dehydrogenase or other enzymes disclosed herein to generate biosynthetic pathways for GBL and/or BDO. Therefore, the BDO producers of the invention can have exogenous expression of, for example, one, two, three, four, five, six, seven, eight, nine, or up to all enzymes corresponding to any of the 4-HB pathway and/or any of the BDO pathway enzymes disclosed herein.

Design and construction of the genetically modified microbial organisms is carried out using methods well known in the art to achieve sufficient amounts of expression to produce BDO. In particular, the non-naturally occurring microbial organisms of the invention can achieve biosynthesis of BDO resulting in intracellular concentrations between about 0.1-200 mM or more, such as about 0.1-25 mM or more, as discussed above. For example, the intracellular concentration of BDO is between about 3-20 mM, particularly between about 5-15 mM and more particularly between about 8-12 mM, including about 10 mM or more. Intracellular concentrations between and above each of these exemplary ranges also can be achieved from the non-naturally occurring microbial organisms of the invention. As with the 4-HB producers, the BDO producers also can be sustained, cultured or fermented under anaerobic conditions.

The invention further provides a method for the production of 4-HB. The method includes culturing a non-naturally occurring microbial organism having a 4-hydroxybutanoic acid (4-HB) biosynthetic pathway comprising at least one exogenous nucleic acid encoding 4-hydroxybutanoate dehydrogenase, CoA-independent succinic semialdehyde dehydrogenase, succinyl-CoA synthetase, CoA-dependent succinic semialdehyde dehydrogenase, glutamate:succinic semialdehyde transaminase, α-ketoglutarate decarboxylase, or glutamate decarboxylase under substantially anaerobic conditions for a sufficient period of time to produce monomeric 4-hydroxybutanoic acid (4-HB). The method can additionally include chemical conversion of 4-HB to GBL and to BDO or THF, for example.

Additionally provided is a method for the production of 4-HB. The method includes culturing a non-naturally occurring microbial organism having a 4-hydroxybutanoic acid (4-HB) biosynthetic pathway including at least one exogenous nucleic acid encoding 4-hydroxybutanoate dehydrogenase, succinyl-CoA synthetase, CoA-dependent succinic semialdehyde dehydrogenase or α-ketoglutarate decarboxylase under substantially anaerobic conditions for a sufficient period of time to produce monomeric 4-hydroxybutanoic acid (4-HB). The 4-HB product can be secreted into the culture medium.

Further provided is a method for the production of BDO. The method includes culturing a non-naturally occurring microbial biocatalyst or microbial organism, comprising a microbial organism having 4-hydroxybutanoic acid (4-HB) and 1,4-butanediol (BDO) biosynthetic pathways, the pathways including at least one exogenous nucleic acid encoding 4-hydroxybutanoate dehydrogenase, succinyl-CoA synthetase, CoA-dependent succinic semialdehyde dehydrogenase, 4-hydroxybutyrate:CoA transferase, 4-hydroxybutyrate kinase, phosphotranshydroxybutyrylase, α-ketoglutarate decarboxylase, aldehyde dehydrogenase, alcohol dehydrogenase or an aldehyde/alcohol dehydrogenase for a sufficient period of time to produce 1,4-butanediol (BDO). The BDO product can be secreted into the culture medium.

Additionally provided are methods for producing BDO by culturing a non-naturally occurring microbial organism having a BDO pathway of the invention. The BDO pathway can comprise at least one exogenous nucleic acid encoding a BDO pathway enzyme expressed in a sufficient amount to produce BDO, under conditions and for a sufficient period of time to produce BDO, the BDO pathway comprising 4-aminobutyrate CoA transferase, 4-aminobutyryl-CoA hydrolase, 4-aminobutyrate-CoA ligase, 4-aminobutyryl-CoA oxidoreductase (deaminating), 4-aminobutyryl-CoA transaminase, or 4-hydroxybutyryl-CoA dehydrogenase (see Example VII; see corresponding Example and Table 17 in WO2013/184602).

Alternatively, the BDO pathway can compre at least one exogenous nucleic acid encoding a BDO pathway enzyme expressed in a sufficient amount to produce BDO, under conditions and for a sufficient period of time to produce BDO, the BDO pathway comprising 4-aminobutyrate CoA transferase, 4-aminobutyryl-CoA hydrolase, 4-aminobutyrate-CoA ligase, 4-aminobutyryl-CoA reductase (alcohol forming), 4-aminobutyryl-CoA reductase, 4-aminobutan-1-ol dehydrogenase, 4-aminobutan-1-ol oxidoreductase (deaminating) or 4-aminobutan-1-ol transaminase (see Example VII; see corresponding Example and Table 18 in WO2013/184602).

In addition, the invention provides a method for producing BDO, comprising culturing a non-naturally occurring microbial organism having a BDO pathway, the pathway comprising at least one exogenous nucleic acid encoding a BDO pathway enzyme expressed in a sufficient amount to produce BDO, under conditions and for a sufficient period of time to produce BDO, the BDO pathway comprising 4-aminobutyrate kinase, 4-aminobutyraldehyde dehydrogenase (phosphorylating), 4-aminobutan-1-ol dehydrogenase, 4-aminobutan-1-ol oxidoreductase (deaminating), 4-aminobutan-1-ol transaminase, [(4-aminobutanolyl)oxy]phosphonic acid oxidoreductase (deaminating), [(4-aminobutanolyl)oxy]phosphonic acid transaminase, 4-hydroxybutyryl-phosphate dehydrogenase, or 4-hydroxybutyraldehyde dehydrogenase (phosphorylating)(see Example VII; see corresponding Example and Table 19 in WO2013/184602).

The invention further provides a method for producing BDO, comprising culturing a non-naturally occurring microbial organism having a BDO pathway, the pathway comprising at least one exogenous nucleic acid encoding a BDO pathway enzyme expressed in a sufficient amount to produce BDO, under conditions and for a sufficient period of time to produce BDO, the BDO pathway comprising alpha-ketoglutarate 5-kinase, 2,5-dioxopentanoic semialdehyde dehydrogenase (phosphorylating), 2,5-dioxopentanoic acid reductase, alpha-ketoglutarate CoA transferase, alpha-ketoglutaryl-CoA hydrolase, alpha-ketoglutaryl-CoA ligase, alpha-ketoglutaryl-CoA reductase, 5-hydroxy-2-oxopentanoic acid dehydrogenase, alpha-ketoglutaryl-CoA reductase (alcohol forming), 5-hydroxy-2-oxopentanoic acid decarboxylase, or 5-hydroxy-2-oxopentanoic acid dehydrogenase (decarboxylation)(see Example VIII; see corresponding Example and Table 20 in WO2013/184602).

The invention additionally provides a method for producing BDO, comprising culturing a non-naturally occurring microbial organism having a BDO pathway, the pathway comprising at least one exogenous nucleic acid encoding a BDO pathway enzyme expressed in a sufficient amount to produce BDO, under conditions and for a sufficient period of time to produce BDO, the BDO pathway comprising glutamate CoA transferase, glutamyl-CoA hydrolase, glutamyl-CoA ligase, glutamate 5-kinase, glutamate-5-semialdehyde dehydrogenase (phosphorylating), glutamyl-CoA reductase, glutamate-5-semialdehyde reductase, glutamyl-CoA reductase (alcohol forming), 2-amino-5-hydroxypentanoic acid oxidoreductase (deaminating), 2-amino-5-hydroxypentanoic acid transaminase, 5-hydroxy-2-oxopentanoic acid decarboxylase, 5-hydroxy-2-oxopentanoic acid dehydrogenase (decarboxylation)(see Example IX; see corresponding Example and Table 21 in WO2013/184602).

The invention additionally includes a method for producing BDO, comprising culturing a non-naturally occurring microbial organism having a BDO pathway, the pathway comprising at least one exogenous nucleic acid encoding a BDO pathway enzyme expressed in a sufficient amount to produce BDO, under conditions and for a sufficient period of time to produce BDO, the BDO pathway comprising 3-hydroxybutyryl-CoA dehydrogenase, 3-hydroxybutyryl-CoA dehydratase, vinylacetyl-CoA Δ-isomerase, or 4-hydroxybutyryl-CoA dehydratase (see Example X; see corresponding Example and Table 22 in WO2013/184602).

Also provided is a method for producing BDO, comprising culturing a non-naturally occurring microbial organism having a BDO pathway, the pathway comprising at least one exogenous nucleic acid encoding a BDO pathway enzyme expressed in a sufficient amount to produce BDO, under conditions and for a sufficient period of time to produce BDO, the BDO pathway comprising homoserine deaminase, homoserine CoA transferase, homoserine-CoA hydrolase, homoserine-CoA ligase, homoserine-CoA deaminase, 4-hydroxybut-2-enoyl-CoA transferase, 4-hydroxybut-2-enoyl-CoA hydrolase, 4-hydroxybut-2-enoyl-CoA ligase, 4-hydroxybut-2-enoate reductase, 4-hydroxybutyryl-CoA transferase, 4-hydroxybutyryl-CoA hydrolase, 4-hydroxybutyryl-CoA ligase, or 4-hydroxybut-2-enoyl-CoA reductase (see Example XI; see corresponding Example and Table 23 in WO2013/184602).

The invention additionally provides a method for producing BDO, comprising culturing a non-naturally occurring microbial organism having a BDO pathway, the pathway comprising at least one exogenous nucleic acid encoding a BDO pathway enzyme expressed in a sufficient amount to produce BDO, under conditions and for a sufficient period of time to produce BDO, the BDO pathway comprising succinyl-CoA reductase (alcohol forming), 4-hydroxybutyryl-CoA hydrolase, 4-hydroxybutyryl-CoA ligase, 4-hydroxybutanal dehydrogenase (phosphorylating). Such a BDO pathway can further comprise succinyl-CoA reductase, 4-hydroxybutyrate dehydrogenase, 4-hydroxybutyryl-CoA transferase, 4-hydroxybutyrate kinase, phosphotrans-4-hydroxybutyrylase, 4-hydroxybutyryl-CoA reductase, 4-hydroxybutyryl-CoA reductase (alcohol forming), or 1,4-butanediol dehydrogenase.

Also provided is a method for producing BDO, comprising culturing a non-naturally occurring microbial organism having a BDO pathway, the pathway comprising at least one exogenous nucleic acid encoding a BDO pathway enzyme expressed in a sufficient amount to produce BDO, under conditions and for a sufficient period of time to produce BDO, the BDO pathway comprising glutamate dehydrogenase, 4-aminobutyrate oxidoreductase (deaminating), 4-aminobutyrate transaminase, glutamate decarboxylase, 4-hydroxybutyryl-CoA hydrolase, 4-hydroxybutyryl-CoA ligase, 4-hydroxybutanal dehydrogenase (phosphorylating).

The invention additionally provides methods of producing a desired product using the genetically modified organisms disclosed herein that allow improved production of a desired product such as BDO by increasing the product or decreasing undesirable byproducts. Thus, the invention provides a method for producing 1,4-butanediol (BDO), comprising culturing the non-naturally occurring microbial organisms disclosed herein under conditions and for a sufficient period of time to produce BDO. In one embodiment, the invention provides a method of producing BDO using a non-naturally occurring microbial organism, comprising a microbial organism having a 1,4-butanediol (BDO) pathway comprising at least one exogenous nucleic acid encoding a BDO pathway enzyme expressed in a sufficient amount to produce BDO. In one embodiment, the microbial organism is genetically modified to express exogenous succinyl-CoA synthetase (see Example XII). For example, the succinyl-CoA synthetase can be encoded by an Escherichia coli sucCD genes.

In another embodiment, the microbial organism is genetically modified to express exogenous alpha-ketoglutarate decarboxylase (see Example XIII). For example, the alpha-ketoglutarate decarboxylase can be encoded by the Mycobacterium bovis sucA gene. In still another embodiment, the microbial organism is genetically modified to express exogenous succinate semialdehyde dehydrogenase and 4-hydroxybutyrate dehydrogenase and optionally 4-hydroxybutyryl-CoA/acetyl-CoA transferase (see Example XIII). For example, the succinate semialdehyde dehydrogenase (CoA-dependent), 4-hydroxybutyrate dehydrogenase and 4-hydroxybutyryl-CoA/acetyl-CoA transferase can be encoded by Porphyromonas gingivalis W83 genes. In an additional embodiment, the microbial organism is genetically modified to express exogenous butyrate kinase and phosphotransbutyrylase (see Example XIII). For example, the butyrate kinase and phosphotransbutyrylase can be encoded by Clostridium acetobutilicum buk1 and ptb genes.

In yet another embodiment, the microbial organism is genetically modified to express exogenous 4-hydroxybutyryl-CoA reductase (see Example XIII). For example, the 4-hydroxybutyryl-CoA reductase can be encoded by Clostridium beijerinckii ald gene. Additionally, in an embodiment of the invention, the microbial organism is genetically modified to express exogenous 4-hydroxybutanal reductase (see Example XIII). For example, the 4-hydroxybutanal reductase can be encoded by Geobacillus thermoglucosidasius adh1 gene. In another embodiment, the microbial organism is genetically modified to express exogenous pyruvate dehydrogenase subunits (see Example XIV). For example, the exogenous pyruvate dehydrogenase can be NADH insensitive. The pyruvate dehydrogenase subunit can be encoded by the Klebsiella pneumonia lpdA gene. In a particular embodiment, the pyruvate dehydrogenase subunit genes of the microbial organism can be under the control of a pyruvate formate lyase promoter.

In still another embodiment, the microbial organism is genetically modified to disrupt a gene encoding an aerobic respiratory control regulatory system (see Example XV). For example, the disruption can be of the arcA gene. Such an organism can further comprise disruption of a gene encoding malate dehydrogenase. In a further embodiment, the microbial organism is genetically modified to express an exogenous NADH insensitive citrate synthase (see Example XV). For example, the NADH insensitive citrate synthase can be encoded by gltA, such as an R163L mutant of gltA. In still another embodiment, the microbial organism is genetically modified to express exogenous phosphoenolpyruvate carboxykinase (see Example XVI). For example, the phosphoenolpyruvate carboxykinase can be encoded by an Haemophilus influenza phosphoenolpyruvate carboxykinase gene. It is understood that strains exemplified herein for improved production of BDO can similarly be used, with appropriate modifications, to produce other desired products, for example, 4-hydroxybutyrate or other desired products disclosed herein.

The invention additionally provides a method for producing 4-hydroxybutanal by culturing a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising succinyl-CoA reductase (aldehyde forming); 4-hydroxybutyrate dehydrogenase; and 4-hydroxybutyrate reductase (see FIG. 58, steps A-C-D). The invention also provides a method for producing 4-hydroxybutanal by culturing a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising alpha-ketoglutarate decarboxylase; 4-hydroxybutyrate dehydrogenase; and 4-hydroxybutyrate reductase (FIG. 58, steps B-C-D).

The invention further provides a method for producing 4-hydroxybutanal by culturing a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising succinate reductase; 4-hydroxybutyrate dehydrogenase, and 4-hydroxybutyrate reductase (see FIG. 62, steps F-C-D). In yet another embodiment, the invention provides a method for producing 4-hydroxybutanal by culturing a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising alpha-ketoglutarate decarboxylase, or glutamate dehydrogenase or glutamate transaminase and glutamate decarboxylase and 4-aminobutyrate dehydrogenase or 4-aminobutyrate transaminase; 4-hydroxybutyrate dehydrogenase; and 4-hydroxybutyrate reductase (see FIG. 62, steps B or ((J or K)-L-(M or N))-C-D).

The invention also provides a method for producing 4-hydroxybutanal by culturing a non-naturally occurring microbial organism, comprising a 4-hydroxybutanal pathway comprising at least one exogenous nucleic acid encoding a 4-hydroxybutanal pathway enzyme expressed in a sufficient amount to produce 4-hydroxybutanal, the 4-hydroxybutanal pathway comprising alpha-ketoglutarate reductase; 5-hydroxy-2-oxopentanoate dehydrogenase; and 5-hydroxy-2-oxopentanoate decarboxylase (see FIG. 62, steps X-Y-Z). The invention further provides a method for producing 4-hydroxybutyryl-CoA by culturing a non-naturally occurring microbial organism, comprising a 4-hydroxybutyryl-CoA pathway comprising at least one exogenous nucleic acid encoding a 4-hydroxybutyryl-CoA pathway enzyme expressed in a sufficient amount to produce 4-hydroxybutyryl-CoA, the 4-hydroxybutyryl-CoA pathway comprising alpha-ketoglutarate reductase; 5-hydroxy-2-oxopentanoate dehydrogenase; and 5-hydroxy-2-oxopentanoate dehydrogenase (decarboxylation)(see FIG. 62, steps X-Y-AA).

The invention additionally provides a method for producing putrescine by culturing a non-naturally occurring microbial organism, comprising a putrescine pathway comprising at least one exogenous nucleic acid encoding a putrescine pathway enzyme expressed in a sufficient amount to produce putrescine, the putrescine pathway comprising succinate reductase; 4-aminobutyrate dehydrogenase or 4-aminobutyrate transaminase; 4-aminobutyrate reductase; and putrescine dehydrogenase or putrescine transaminase (see FIG. 63, steps F-M/N-C-D/E). In still another embodiment, the invention provides a method for producing putrescine by culturing a non-naturally occurring microbial organism, comprising a putrescine pathway comprising at least one exogenous nucleic acid encoding a putrescine pathway enzyme expressed in a sufficient amount to produce putrescine, the putrescine pathway comprising alpha-ketoglutarate decarboxylase; 4-aminobutyrate dehydrogenase or 4-aminobutyrate transaminase; 4-aminobutyrate reductase; and putrescine dehydrogenase or putrescine transaminase (see FIG. 63, steps B-M/N-C-D/E). The invention additionally provides a method for producing putrescine by culturing a non-naturally occurring microbial organism, comprising a putrescine pathway comprising at least one exogenous nucleic acid encoding a putrescine pathway enzyme expressed in a sufficient amount to produce putrescine, the putrescine pathway comprising glutamate dehydrogenase or glutamate transaminase; glutamate decarboxylase; 4-aminobutyrate reductase; and putrescine dehydrogenase or putrescine transaminase (see FIG. 63, steps J/K-L-C-D/E).

The invention provides in another embodiment a method for producing putrescine by culturing a non-naturally occurring microbial organism, comprising a putrescine pathway comprising at least one exogenous nucleic acid encoding a putrescine pathway enzyme expressed in a sufficient amount to produce putrescine, the putrescine pathway comprising alpha-ketoglutarate reductase; 5-amino-2-oxopentanoate dehydrogenase or 5-amino-2-oxopentanoate transaminase; 5-amino-2-oxopentanoate decarboxylase; and putrescine dehydrogenase or putrescine transaminase (see FIG. 63, steps O-P/Q-R-D/E). Also provided is a method for producing putrescine by culturing a non-naturally occurring microbial organism, comprising a putrescine pathway comprising at least one exogenous nucleic acid encoding a putrescine pathway enzyme expressed in a sufficient amount to produce putrescine, the putrescine pathway comprising alpha-ketoglutarate reductase; 5-amino-2-oxopentanoate dehydrogenase or 5-amino-2-oxopentanoate transaminase; ornithine dehydrogenase or ornithine transaminase; and ornithine decarboxylase (see FIG. 63, steps O-P/Q-S/T-U). It is understood that a microbial organism comprising any of the pathways disclosed herein can be used to produce a desired product or intermediate, including 4-HB, 4-HBal, BDO or putrescine.

It is understood that, in methods of the invention, any of the one or more exogenous nucleic acids can be introduced into a microbial organism to produce a non-naturally occurring microbial organism of the invention. The nucleic acids can be introduced so as to confer, for example, a 4-HB, BDO, THF or GBL biosynthetic pathway onto the microbial organism. Alternatively, encoding nucleic acids can be introduced to produce an intermediate microbial organism having the biosynthetic capability to catalyze some of the required reactions to confer 4-HB, BDO, THF or GBL biosynthetic capability. For example, a non-naturally occurring microbial organism having a 4-HB biosynthetic pathway can comprise at least two exogenous nucleic acids encoding desired enzymes, such as the combination of 4-hydroxybutanoate dehydrogenase and α-ketoglutarate decarboxylase; 4-hydroxybutanoate dehydrogenase and CoA-independent succinic semialdehyde dehydrogenase; 4-hydroxybutanoate dehydrogenase and CoA-dependent succinic semialdehyde dehydrogenase; CoA-dependent succinic semialdehyde dehydrogenase and succinyl-CoA synthetase; succinyl-CoA synthetase and glutamate decarboxylase, and the like. Thus, it is understood that any combination of two or more enzymes of a biosynthetic pathway can be included in a non-naturally occurring microbial organism of the invention. Similarly, it is understood that any combination of three or more enzymes of a biosynthetic pathway can be included in a non-naturally occurring microbial organism of the invention, for example, 4-hydroxybutanoate dehydrogenase, α-ketoglutarate decarboxylase and CoA-dependent succinic semialdehyde dehydrogenase; CoA-independent succinic semialdehyde dehydrogenase and succinyl-CoA synthetase; 4-hydroxybutanoate dehydrogenase, CoA-dependent succinic semialdehyde dehydrogenase and glutamate:succinic semialdehyde transaminase, and so forth, as desired, so long as the combination of enzymes of the desired biosynthetic pathway results in production of the corresponding desired product.

Similarly, for example, with respect to any one or more exogenous nucleic acids introduced to confer BDO production, a non-naturally occurring microbial organism having a BDO biosynthetic pathway can comprise at least two exogenous nucleic acids encoding desired enzymes, such as the combination of 4-hydroxybutanoate dehydrogenase and α-ketoglutarate decarboxylase; 4-hydroxybutanoate dehydrogenase and 4-hydroxybutyryl CoA:acetyl-CoA transferase; 4-hydroxybutanoate dehydrogenase and butyrate kinase; 4-hydroxybutanoate dehydrogenase and phosphotransbutyrylase; 4-hydroxybutyryl CoA:acetyl-CoA transferase and aldehyde dehydrogenase; 4-hydroxybutyryl CoA:acetyl-CoA transferase and alcohol dehydrogenase; 4-hydroxybutyryl CoA:acetyl-CoA transferase and an aldehyde/alcohol dehydrogenase, 4-aminobutyrate-CoA transferase and 4-aminobutyryl-CoA transaminase; 4-aminobutyrate kinase and 4-aminobutan-1-ol oxidoreductase (deaminating), and the like. Thus, it is understood that any combination of two or more enzymes of a biosynthetic pathway can be included in a non-naturally occurring microbial organism of the invention. Similarly, it is understood that any combination of three or more enzymes of a biosynthetic pathway can be included in a non-naturally occurring microbial organism of the invention, for example, 4-hydroxybutanoate dehydrogenase, α-ketoglutarate decarboxylase and 4-hydroxybutyryl CoA:acetyl-CoA transferase; 4-hydroxybutanoate dehydrogenase, butyrate kinase and phosphotransbutyrylase; 4-hydroxybutanoate dehydrogenase, 4-hydroxybutyryl CoA:acetyl-CoA transferase and aldehyde dehydrogenase; 4-hydroxybutyryl CoA:acetyl-CoA transferase, aldehyde dehydrogenase and alcohol dehydrogenase; butyrate kinase, phosphotransbutyrylase and an aldehyde/alcohol dehydrogenase; 4-aminobutyryl-CoA hydrolase, 4-aminobutyryl-CoA reductase and 4-amino butan-1-ol transaminase; 3-hydroxybutyryl-CoA dehydrogenase, 3-hydroxybutyryl-CoA dehydratase and 4-hydroxybutyryl-CoA dehydratase, and the like. Similarly, any combination of four, five or more enzymes of a biosynthetic pathway as disclosed herein can be included in a non-naturally occurring microbial organism of the invention, as desired, so long as the combination of enzymes of the desired biosynthetic pathway results in production of the corresponding desired product.

Any of the non-naturally occurring microbial organisms described herein can be cultured to produce and/or secrete the biosynthetic products of the invention. For example, the 4-HB producers can be cultured for the biosynthetic production of 4-HB. The 4-HB can be isolated or be treated as described below to generate GBL, THF and/or BDO. Similarly, the BDO producers can be cultured for the biosynthetic production of BDO. The BDO can be isolated or subjected to further treatments for the chemical synthesis of BDO family compounds, as disclosed herein. Thus, the invention provides a method of producing a desired product such as 4-HB, 4-Bal, 4-HBCoA, BDO or putrescine, for example, 4-HB or BDO using a microbial organism of the invention. Optionally, a purification or isolation step can be applied, for example, distillation, to purify the product such as 4-HB, 4-HBal, 4-HBCoA, BDO or putrescine, for example, 4-hydroxybutyrate or 1,4-butanediol. Thus, the invention also provides a method for producing isolated or purified product such as 4-HB, 4-HBal, 4-HBCoA, BDO or putrescine, for example, 4-hydroxybutyrate or 1,4-butanediol, by culturing the non-naturally occurring microbial organism of the invention under conditions and for a sufficient period of time to produce the product such as 4-HB, 4-HBal, 4-HBCoA, BDO or putrescine, for example, 4-hydroxybutyrate or 1,4-butanediol, and isolating or purifying the product. The isolating or purifying can comprise a step such as distillation.

The growth medium can include, for example, any carbohydrate source which can supply a source of carbon to the non-naturally occurring microorganism. Such sources include, for example, sugars such as glucose, sucrose, xylose, arabinose, galactose, mannose, fructose and starch; or glycerol, and it is understood that a carbon source can be used alone as the sole source of carbon or in combination with other carbon sources described herein or known in the art. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, sucrose, xylose, arabinose, galactose, mannose, fructose and starch. Given the teachings and guidance provided herein, those skilled in the art will understand that renewable feedstocks and biomass other than those exemplified above also can be used for culturing the microbial organisms of the invention for the production of 4-HB, 4-HBal, 4-HBCoA, BDO or putrescine and other compounds of the invention.

Accordingly, given the teachings and guidance provided herein, those skilled in the art will understand that a non-naturally occurring microbial organism can be produced that secretes the biosynthesized compounds of the invention when grown on a carbon source such as a carbohydrate. Such compounds include, for example, 4-HB, 4-HBal, 4-HBCoA, BDO or putrescine and any of the intermediates metabolites in the 4-HB, 4-HBal, 4-HBCoA, BDO or putrescine pathways and/or the combined 4-HB, 4-HBal, 4-HBCoA, BDO or putrescine pathways. All that is required is to engineer in one or more of the enzyme activities shown in FIG. 1 to achieve biosynthesis of the desired compound or intermediate including, for example, inclusion of some or all of the 4-HB, 4-HBal, 4-HBCoA, BDO or putrescine biosynthetic pathways. Accordingly, the invention provides a non-naturally occurring microbial organism that secretes 4-HB when grown on a carbohydrate, secretes BDO when grown on a carbohydrate and/or secretes any of the intermediate metabolites shown in FIGS. 1, 8-13, 58, 62, 63, 69 and 70 when grown on a carbohydrate. ABDO producing microbial organisms of the invention can initiate synthesis from, for example, succinate, succinyl-CoA, α-ketogluterate, succinic semialdehyde, 4-HB, 4-hydroxybutyrylphosphate, 4-hydroxybutyryl-CoA (4-HB-CoA) and/or 4-hydroxybutyraldehyde.

In some embodiments, culture conditions include anaerobic or substantially anaerobic growth or maintenance conditions. Exemplary anaerobic conditions have been described previously and are well known in the art. Exemplary anaerobic conditions for fermentation processes are described below in the Examples. Any of these conditions can be employed with the non-naturally occurring microbial organisms as well as other anaerobic conditions well known in the art. Under such anaerobic conditions, the 4-HB, 4-HBal, 4-HBCoA, BDO or putrescine producers can synthesize monomeric 4-HB, 4-HBal, 4-HBCoA, BDO or putrescine, respectively, at intracellular concentrations of 5-10 mM or more as well as all other concentrations exemplified previously.

A number of downstream compounds also can be generated for the 4-HB, 4-HBal, 4-HBCoA, BDO or putrescine producing non-naturally occurring microbial organisms of the invention. With respect to the 4-HB producing microbial organisms of the invention, monomeric 4-HB and GBL exist in equilibrium in the culture medium. The conversion of 4-HB to GBL can be efficiently accomplished by, for example, culturing the microbial organisms in acid pH medium. A pH less than or equal to 7.5, in particular at or below pH 5.5, spontaneously converts 4-HB to GBL.

The resultant GBL can be separated from 4-HB and other components in the culture using a variety of methods well known in the art. Such separation methods include, for example, the extraction procedures exemplified in the Examples as well as methods which include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art. Separated GBL can be further purified by, for example, distillation.

Another down stream compound that can be produced from the 4-HB producing non-naturally occurring microbial organisms of the invention includes, for example, BDO. This compound can be synthesized by, for example, chemical hydrogenation of GBL. Chemical hydrogenation reactions are well known in the art. One exemplary procedure includes the chemical reduction of 4-HB and/or GBL or a mixture of these two components deriving from the culture using a heterogeneous or homogeneous hydrogenation catalyst together with hydrogen, or a hydride-based reducing agent used stoichiometrically or catalytically, to produce 1,4-butanediol.

Other procedures well known in the art are equally applicable for the above chemical reaction and include, for example, WO No. 82/03854 (Bradley, et al.), which describes the hydrogenolysis of gamma-butyrolactone in the vapor phase over a copper oxide and zinc oxide catalyst. British Pat. No. 1,230,276, which describes the hydrogenation of gamma-butyrolactone using a copper oxide-chromium oxide catalyst. The hydrogenation is carried out in the liquid phase. Batch reactions also are exemplified having high total reactor pressures. Reactant and product partial pressures in the reactors are well above the respective dew points. British Pat. No. 1,314,126, which describes the hydrogenation of gamma-butyrolactone in the liquid phase over a nickel-cobalt-thorium oxide catalyst. Batch reactions are exemplified as having high total pressures and component partial pressures well above respective component dew points. British Pat. No. 1,344,557, which describes the hydrogenation of gamma-butyrolactone in the liquid phase over a copper oxide-chromium oxide catalyst. A vapor phase or vapor-containing mixed phase is indicated as suitable in some instances. A continuous flow tubular reactor is exemplified using high total reactor pressures. British Pat. No. 1,512,751, which describes the hydrogenation of gamma-butyrolactone to 1,4-butanediol in the liquid phase over a copper oxide-chromium oxide catalyst. Batch reactions are exemplified with high total reactor pressures and, where determinable, reactant and product partial pressures well above the respective dew points. U.S. Pat. No. 4,301,077, which describes the hydrogenation to 1,4-butanediol of gamma-butyrolactone over a Ru—Ni—Co—Zn catalyst. The reaction can be conducted in the liquid or gas phase or in a mixed liquid-gas phase. Exemplified are continuous flow liquid phase reactions at high total reactor pressures and relatively low reactor productivities. U.S. Pat. No. 4,048,196, which describes the production of 1,4-butanediol by the liquid phase hydrogenation of gamma-butyrolactone over a copper oxide-zinc oxide catalyst. Further exemplified is a continuous flow tubular reactor operating at high total reactor pressures and high reactant and product partial pressures. And U.S. Pat. No. 4,652,685, which describes the hydrogenation of lactones to glycols.

A further downstream compound that can be produced form the 4-HB producing microbial organisms of the invention includes, for example, THF. This compound can be synthesized by, for example, chemical hydrogenation of GBL. One exemplary procedure well known in the art applicable for the conversion of GBL to THF includes, for example, chemical reduction of 4-HB and/or GBL or a mixture of these two components deriving from the culture using a heterogeneous or homogeneous hydrogenation catalyst together with hydrogen, or a hydride-based reducing agent used stoichiometrically or catalytically, to produce tetrahydrofuran. Other procedures well know in the art are equally applicable for the above chemical reaction and include, for example, U.S. Pat. No. 6,686,310, which describes high surface area sol-gel route prepared hydrogenation catalysts. Processes for the reduction of maleic acid to tetrahydrofuran (THF) and 1,4-butanediol (BDO) and for the reduction of gamma butyrolactone to tetrahydrofuran and 1,4-butanediol also are described.

The culture conditions can include, for example, liquid culture procedures as well as fermentation and other large scale culture procedures. As described further below in the Examples, particularly useful yields of the biosynthetic products of the invention can be obtained under anaerobic or substantially anaerobic culture conditions.

Suitable purification and/or assays to test for the production of 4-HB, 4-HBal, 4-HBCoA, BDO or putrescine can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art. In one embodiment, the invention provides a method that is an assay, wherein a first enzyme is used to generate a substrate for a second test enzyme. In particular, a carboxylic acid transferase 2 (cat2), also referred to herein as a 4-hydroxybutyrate coenzyme A transferase, is used to generate 4-hydroxybutyrl-CoA, which serves as a substrate for aldehyde dehydrogenase (see Example XXXXII). Such an assay can be conveniently used to generate a substrate, in particular for an enzyme-coupled in vitro assay. The method includes providing a carboxylic acid transferase 2, with an appropriate substrate such as 4-hydroxybutyrate, and a test enzyme such as an aldehyde dehydrogenase. It is understood that such a method can utilize well known methods of testing for product formation (see Example XXXXIII).

The 4-HB, 4-HBal, 4-HBCoA, BDO or putrescine product can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

The invention further provides a method of manufacturing 4-HB. The method includes fermenting a non-naturally occurring microbial organism having a 4-hydroxybutanoic acid (4-HB) biosynthetic pathway comprising at least one exogenous nucleic acid encoding 4-hydroxybutanoate dehydrogenase, CoA-independent succinic semialdehyde dehydrogenase, succinyl-CoA synthetase, CoA-dependent succinic semialdehyde dehydrogenase, glutamate:succinic semialdehyde transaminase, α-ketoglutarate decarboxylase, or glutamate decarboxylase under substantially anaerobic conditions for a sufficient period of time to produce monomeric 4-hydroxybutanoic acid (4-HB), the process comprising fed-batch fermentation and batch separation; fed-batch fermentation and continuous separation, or continuous fermentation and continuous separation.

The culture and chemical hydrogenations described above also can be scaled up and grown continuously for manufacturing of 4-HB, 4-HBal, 4-HBCoA, GBL, BDO and/or THF or putrescine. Exemplary growth procedures include, for example, fed-batch fermentation and batch separation; fed-batch fermentation and continuous separation, or continuous fermentation and continuous separation. All of these processes are well known in the art. Employing the 4-HB producers allows for simultaneous 4-HB biosynthesis and chemical conversion to GBL, BDO and/or THF by employing the above hydrogenation procedures simultaneous with continuous cultures methods such as fermentation. Other hydrogenation procedures also are well known in the art and can be equally applied to the methods of the invention.

Fermentation procedures are particularly useful for the biosynthetic production of commercial quantities of 4-HB, 4-HBal, 4-HBCoA, BDO or putrescine. Generally, and as with non-continuous culture procedures, the continuous and/or near-continuous production of 4-HB, 4-HBal, 4-HBCoA, BDO or putrescine will include culturing a non-naturally occurring 4-HB, 4-HBal, 4-HBCoA, BDO or putrescine producing organism of the invention in sufficient nutrients and medium to sustain and/or nearly sustain growth in an exponential phase. Continuous culture under such conditions can be include, for example, growth or culturing 1 day, 2, 3, 4, 5, 6 or 7 days or more. Additionally, continuous culture can include longer time periods 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of 4-HB, 4-HBal, 4-HBCoA, BDO or putrescine or other 4-HB, 4-HBal, 4-HBCoA, BDO or putrescine derived products, including intermediates, of the invention can be utilized in, for example, fed-batch fermentation and batch separation; fed-batch fermentation and continuous separation, or continuous fermentation and continuous separation. Examples of batch and continuous fermentation procedures well known in the art are exemplified further below in the Examples.

In addition to the above fermentation procedures using the 4-HB, 4-HBal, 4-HBCoA, BDO or putrescine producers of the invention for continuous production of substantial quantities of 4-HB, 4-HBal, 4-HBCoA, BDO or putrescine, including monomeric 4-HB, respectively, the 4-HB producers also can be, for example, simultaneously subjected to chemical synthesis procedures to convert the product to other compounds or the product as described previously for the chemical conversion of monomeric 4-HB to, for example, GBL, BDO and/or THF. The BDO producers can similarly be, for example, simultaneously subjected to chemical synthesis procedures as described previously for the chemical conversion of BDO to, for example, THF, GBL, pyrrolidones and/or other BDO family compounds. In addition, the products of the 4-HB, 4-HBal, 4-HBCoA, BDO or putrescine producers can be separated from the fermentation culture and sequentially subjected to chemical or enzymatic conversion to convert the product to other compounds, if desired, as disclosed herein.

Briefly, hydrogenation of GBL in the fermentation broth can be performed as described by Frost et al., Biotechnology Progress 18: 201-211 (2002). Another procedure for hydrogenation during fermentation include, for example, the methods described in, for example, U.S. Pat. No. 5,478,952. This method is further exemplified in the Examples below.

Therefore, the invention additionally provides a method of manufacturing γ-butyrolactone (GBL), tetrahydrofuran (THF) or 1,4-butanediol (BDO). The method includes fermenting a non-naturally occurring microbial organism having 4-hydroxybutanoic acid (4-HB) and/or 1,4-butanediol (BDO) biosynthetic pathways, the pathways comprise at least one exogenous nucleic acid encoding 4-hydroxybutanoate dehydrogenase, CoA-independent succinic semialdehyde dehydrogenase, succinyl-CoA synthetase, CoA-dependent succinic semialdehyde dehydrogenase, 4-hydroxybutyrate:CoA transferase, glutamate:succinic semialdehyde transaminase, α-ketoglutarate decarboxylase, glutamate decarboxylase, 4-hydroxybutanoate kinase, phosphotransbutyrylase, CoA-independent 1,4-butanediol semialdehyde dehydrogenase, CoA-dependent 1,4-butanediol semialdehyde dehydrogenase, CoA-independent 1,4-butanediol alcohol dehydrogenase or CoA-dependent 1,4-butanediol alcohol dehydrogenase, under substantially anaerobic conditions for a sufficient period of time to produce 1,4-butanediol (BDO), GBL or THF, the fermenting comprising fed-batch fermentation and batch separation; fed-batch fermentation and continuous separation, or continuous fermentation and continuous separation.

In addition to the biosynthesis of 4-HB, 4-HBal, 4-HBCoA, BDO or putrescine and other products of the invention as described herein, the non-naturally occurring microbial organisms and methods of the invention also can be utilized in various combinations with each other and/or with other microbial organisms and methods well known in the art to achieve product biosynthesis by other routes. For example, one alternative to produce BDO other than use of the 4-HB producers and chemical steps or other than use of the BDO producer directly is through addition of another microbial organism capable of converting 4-HB or a 4-HB product exemplified herein to BDO.

One such procedure includes, for example, the fermentation of a 4-B producing microbial organism of the invention to produce 4-HB, as described above and below. The 4-HB can then be used as a substrate for a second microbial organism that converts 4-HB to, for example, BDO, GBL and/or THF. The 4-HB can be added directly to another culture of the second organism or the original culture of 4-HB producers can be depleted of these microbial organisms by, for example, cell separation, and then subsequent addition of the second organism to the fermentation broth can utilized to produce the final product without intermediate purification steps. One exemplary second organism having the capacity to biochemically utilize 4-HB as a substrate for conversion to BDO, for example, is Clostridium acetobutylicum (see, for example, Jewell et al., Current Microbiology, 13:215-19 (1986)).

Thus, such a procedure includes, for example, the fermentation of a microbial organism that produces a 4-HB, 4-HBal, 4-HBCoA, BDO or putrescine pathway intermediate. The 4-HB, 4-HBal, 4-HBCoA, BDO or putrescine pathway intermediate can then be used as a substrate for a second microbial organism that converts the 4-HB, 4-HBal, 4-HBCoA, BDO or putrescine pathway intermediate to 4-HB, 4-HBal, 4-HBCoA, BDO or putrescine. The 4-HB, 4-HBal, 4-HBCoA, BDO or putrescine pathway intermediate can be added directly to another culture of the second organism or the original culture of the 4-HB, 4-HBal, 4-HBCoA BDO or putrescine pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods of the invention can be assembled in a wide variety of subpathways to achieve biosynthesis of, for example, 4-HB and/or BDO and/or other pathway intermediates as described. In these embodiments, biosynthetic pathways for a desired product of the invention can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of BDO can be accomplished as described herein by constructing a microbial organism that contains biosynthetic pathways for conversion of one pathway intermediate to another pathway intermediate or the product, for example, a substrate such as endogenous succinate through 4-HB to the final product BDO. Alternatively, BDO also can be biosynthetically produced from microbial organisms through co-culture or co-fermentation using two organisms in the same vessel. A first microbial organism being a 4-HB producer with genes to produce 4-HB from succinic acid, and a second microbial organism being a BDO producer with genes to convert 4-HB to BDO. For example, the biosynthesis of 4-HB, 4-HBal, 4-HBCoA, BDO or putrescine can be accomplished by constructing a microbial organism that contains biosynthetic pathways for conversion of one pathway intermediate to another pathway intermediate or the product. Alternatively, 4-HB, 4-HBal, 4-HBCoA, BDO or putrescine also can be biosynthetically produced from microbial organisms through co-culture or co-fermentation using two organisms in the same vessel, where the first microbial organism produces a 4-HB, 4-HBal, 4-HBCoA, BDO or putrescine intermediate and the second microbial organism converts the intermediate to 4-HB, 4-Bal, 4-HBCoA, BDO or putrescine.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce 4-HB, 4-HBal, BDO, GBL, THF and putrescine products of the invention.

Similarly, it is understood by those skilled in the art that a host organism can be selected based on desired characteristics for introduction of one or more gene disruptions to increase production of 4-HB, 4-HBal, 4-HBCoA, BDO or putrescine. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods of the invention can be applied to any suitable host microorganism to identify the cognate metabolic alterations needed to construct an organism in a species of interest that will increase 4-HB, 4-HBal, 4-HBCoA, BDO or putrescine biosynthesis. In a particular embodiment, the increased production couples biosynthesis of 4-HB, 4-HBal, 4-HBCoA, BDO or putrescine to growth of the organism, and can obligatorily couple production of 4-HB, 4-HBal, 4-HBCoA, BDO or putrescine to growth of the organism if desired and as disclosed herein.

It is understood that, in methods of the invention, any of the one or more exogenous nucleic acids can be introduced into a microbial organism to produce a non-naturally occurring microbial organism of the invention. The nucleic acids can be introduced so as to confer, for example, a 4-HB, 4-HBal, 4-HBCoA, BDO or putrescine biosynthetic pathway onto the microbial organism. Alternatively, encoding nucleic acids can be introduced to produce an intermediate microbial organism having the biosynthetic capability to catalyze some of the required reactions to confer 4-HB, 4-HBal, 4-HBCoA, BDO or putrescine biosynthetic capability. For example, a non-naturally occurring microbial organism having a 4-HB, 4-HBal, 4-HBCoA, BDO or putrescine biosynthetic pathway can comprise at least two exogenous nucleic acids encoding desired enzymes or proteins, such as the combination of enzymes as disclosed herein (see Examples and FIGS. 1, 8-13, 58, 62, 63, 69 and 70), and the like. Thus, it is understood that any combination of two or more enzymes or proteins of a biosynthetic pathway can be included in a non-naturally occurring microbial organism of the invention. Similarly, it is understood that any combination of three or more enzymes or proteins of a biosynthetic pathway can be included in a non-naturally occurring microbial organism of the invention, for example,], and so forth, as desired and disclosed herein, so long as the combination of enzymes and/or proteins of the desired biosynthetic pathway results in production of the corresponding desired product. Similarly, any combination of four or more enzymes or proteins of a biosynthetic pathway as disclosed herein can be included in a non-naturally occurring microbial organism of the invention, as desired, so long as the combination of enzymes and/or proteins of the desired biosynthetic pathway results in production of the corresponding desired product.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of 4-HB, 4-HBal, 4-HBCoA, BDO or putrescine.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion or disruption strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

The methods exemplified above and further illustrated in the Examples below allow the construction of cells and organisms that biosynthetically produce, including obligatory couple production of a target biochemical product to growth of the cell or organism engineered to harbor the identified genetic alterations. In this regard, metabolic alterations have been identified that result in the biosynthesis of 4-HB and 1,4-butanediol. Microorganism strains constructed with the identified metabolic alterations produce elevated levels of 4-HB, 4-HBal, 4-HBCoA, BDO or putrescine compared to unmodified microbial organisms. These strains can be beneficially used for the commercial production of 4-B, BDO, THF, GBL, 4-HBal, 4-HBCoA or putrescine, for example, in continuous fermentation process without being subjected to the negative selective pressures.

Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

Any of the non-naturally occurring microbial organisms described herein can be cultured to produce and/or secrete the biosynthetic products of the invention. For example, the 4-HB, 4-HBal, 4-HBCoA, BDO or putrescine producers can be cultured for the biosynthetic production of 4-HB, 4-HBal, 4-HBCoA, BDO or putrescine. Accordingly, in some embodiments, the invention provides culture medium having 4-HB, 4-HBal, 4-HBCoA, BDO or putrescine or a 4-HB, 4-Bal, 4-HBCoA, BDO or putrescine pathway intermediate described herein. In some aspects, the culture medium can also be separated from the non-naturally occurring microbial organisms of the invention that produced the 4-HB, 4-HBal, 4-HBCoA, BDO or putrescine or 4-HB, 4-HBal, 4-HBCoA, BDO or putrescine pathway intermediate. Methods for separating a microbial organism from culture medium are well known in the art. Exemplary methods include filtration, flocculation, precipitation, centrifugation, sedimentation, and the like.

For the production of 4-HB, 4-HBal, 4-HBCoA, BDO or putrescine, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in U.S. publication 2009/0047719, filed Aug. 10, 2007. Fermentations can be performed in a batch, fed-batch or continuous manner, as disclosed herein. Fermentations can also be conducted in two phases, if desired. The first phase can be aerobic to allow for high growth and therefore high productivity, followed by an anaerobic phase of high 4-HB, 4-HBal, 4-HBCoA, BDO or putrescine yields.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

In addition to renewable feedstocks such as those exemplified above, the 4-HB, 4-HBal, 4-HBCoA, BDO or putrescine producing microbial organisms of the invention also can be modified for growth on syngas as its source of carbon. In this specific embodiment, one or more proteins or enzymes are expressed in the 4-HB, 4-HBal, 4-HBCoA, BDO or putrescine producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

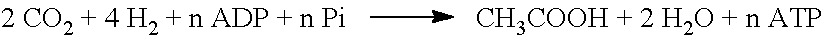

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a 4-HB, 4-HBal, 4-HBCoA, BDO or putrescine pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

Additionally, the reductive (reverse)tricarboxylic acid cycle coupled with carbon monoxide dehydrogenase and/or hydrogenase activities can also be used for the conversion of CO, CO₂ and/or H₂ to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, NAD(P)H:ferredoxin oxidoreductase, carbon monoxide dehydrogenase, and hydrogenase. Specifically, the reducing equivalents extracted from CO and/or H₂ by carbon monoxide dehydrogenase and hydrogenase are utilized to fix CO₂ via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to the 4-HB, 4-HBal, 4-HBCoA, BDO or putrescine precursors, glyceraldehyde-3-phosphate, phosphoenolpyruvate, and pyruvate, by pyruvate:ferredoxin oxidoreductase and the enzymes of gluconeogenesis. Following the teachings and guidance provided herein for introducing a sufficient number of encoding nucleic acids to generate a 4-HB, 4-HBal, 4-HBCoA, BDO or putrescine pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms of the invention such that the modified organism contains the complete reductive TCA pathway will confer syngas utilization ability.

Accordingly, given the teachings and guidance provided herein, those skilled in the art will understand that a non-naturally occurring microbial organism can be produced that secretes the biosynthesized compounds of the invention when grown on a carbon source such as a carbohydrate. Such compounds include, for example, 4-HB, 4-HBal, 4-HBCoA, BDO or putrescine and any of the intermediate metabolites in the 4-HB, 4-HBal, 4-HBCoA, BDO or putrescine pathway. All that is required is to engineer in one or more of the required enzyme or protein activities to achieve biosynthesis of the desired compound or intermediate including, for example, inclusion of some or all of the 4-HB, 4-HBal, 4-HBCoA, BDO or putrescine biosynthetic pathways. Accordingly, the invention provides a non-naturally occurring microbial organism that produces and/or secretes 4-HB, 4-HBal, 4-HBCoA, BDO or putrescine when grown on a carbohydrate or other carbon source and produces and/or secretes any of the intermediate metabolites shown in the 4-HB, 4-HBal, 4-HBCoA, BDO or putrescine pathway when grown on a carbohydrate or other carbon source. The 4-HB, 4-HBal, 4-HBCoA, BDO or putrescine producing microbial organisms of the invention can initiate synthesis from an intermediate in a 4-HB, 4-HBal, 4-HBCoA, BDO or putrescine pathway, as disclosed herein.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of 4-HB, 4-HBal, 4-HBCoA, BDO or putrescine.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003 (WO/2003/106998). SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

Employing the methods exemplified above and herein, the methods of the invention allow the construction of cells and organisms that increase production of a desired product, for example, by coupling the production of a desired product to growth of the cell or organism engineered to harbor the identified genetic alterations. As disclosed herein, metabolic alterations have been identified that couple the production of 4-HB, 4-HBal, 4-HBCoA, BDO or putrescine to growth of the organism. Microbial organism strains constructed with the identified metabolic alterations produce elevated levels, relative to the absence of the metabolic alterations, of 4-HB, 4-HBal, 4-HBCoA, BDO or putrescine during the exponential growth phase. These strains can be beneficially used for the commercial production of 4-HB, 4-HBal, 4-HBCoA, BDO or putrescine in continuous fermentation process without being subjected to the negative selective pressures described previously. Although exemplified herein as metabolic alterations, in particular one or more gene disruptions, that confer growth coupled production of 4-HB, 4-HBal, 4-HBCoA, BDO or putrescine, it is understood that any gene disruption that increases the production of 4-HB, 4-HBal, 4-HBCoA, BDO or putrescine can be introduced into a host microbial organism, as desired.

Therefore, the methods of the invention provide a set of metabolic modifications that are identified by an in silico method such as OptKnock. The set of metabolic modifications can include functional disruption of one or more metabolic reactions including, for example, disruption by gene deletion. For 4-HB, 4-HBal, 4-HBCoA, BDO or putrescine production, metabolic modifications can be selected from the set of metabolic modifications described herein, including the Examples.

Also provided is a method of producing a non-naturally occurring microbial organisms having stable growth-coupled production of 4-HB, 4-HBal, 4-HBCoA, BDO or putrescine. The method can include identifying in silico a set of metabolic modifications that increase production of 4-HB, 4-HBal, 4-HBCoA, BDO or putrescine, for example, increase production during exponential growth; genetically modifying an organism to contain the set of metabolic modifications that increase production of 4-HB, 4-HBal, 4-HBCoA, BDO or putrescine, and culturing the genetically modified organism. If desired, culturing can include adaptively evolving the genetically modified organism under conditions requiring production of 4-B, 4-HBal, 4-HBCoA, BDO or putrescine. The methods of the invention are applicable to bacterium, yeast and fungus as well as a variety of other cells and microorganism, as disclosed herein.

Thus, the invention provides a non-naturally occurring microbial organism comprising one or more gene disruptions that confer increased production of 4-HB, 4-HBal, 4-HBCoA, BDO or putrescine. In one embodiment, the one or more gene disruptions confer growth-coupled production of 4-HB, 4-HBal, 4-HBCoA, BDO or putrescine, and can, for example, confer stable growth-coupled production of 4-HB, 4-HBal, 4-HBCoA, BDO or putrescine. In another embodiment, the one or more gene disruptions can confer obligatory coupling of 4-B, 4-HBal, 4-HBCoA, BDO or putrescine production to growth of the microbial organism. Such one or more gene disruptions reduce the activity of the respective one or more encoded enzymes.

The non-naturally occurring microbial organism can have one or more gene disruptions included in a metabolic modification as described herein. As disclosed herein, the one or more gene disruptions can be a deletion. Such non-naturally occurring microbial organisms of the invention include bacteria, yeast, fungus, or any of a variety of other microorganisms applicable to fermentation processes, as disclosed herein.

Thus, the invention provides a non-naturally occurring microbial organism, comprising one or more gene disruptions, where the one or more gene disruptions occur in genes encoding proteins or enzymes where the one or more gene disruptions confer increased production of 4-HB, 4-HBal, 4-HBCoA, BDO or putrescine in the organism. The production of 4-B, 4-HBal, 4-HBCoA, BDO or putrescine can be growth-coupled or not growth-coupled. In a particular embodiment, the production of 4-HB, 4-HBal, 4-HBCoA, BDO or putrescine can be obligatorily coupled to growth of the organism, as disclosed herein.

The invention provides non naturally occurring microbial organisms having genetic alterations such as gene disruptions that increase production of 4-HB, 4-HBal, 4-HBCoA, BDO or putrescine, for example, growth-coupled production of 4-HB, 4-HBal, 4-HBCoA, BDO or putrescine. Product production can be, for example, obligatorily linked to the exponential growth phase of the microorganism by genetically altering the metabolic pathways of the cell, as disclosed herein. The genetic alterations can increase the production of the desired product or even make the desired product an obligatory product during the growth phase. Sets of metabolic alterations or transformations that result in increased production and elevated levels of 4-HB, 4-HBal, 4-HBCoA, BDO or putrescine biosynthesis are described herein. Each alteration within a set corresponds to the requisite metabolic reaction that should be functionally disrupted. Functional disruption of all reactions within each set can result in the increased production of 4-HB, 4-HBal, 4-HBCoA, BDO or putrescine by the engineered strain during the growth phase.

A number of metabolic modifications that include gene disruptions are described herein. It is understood by those skilled in the art that one or more of the metabolic modifications, including gene disruptions, can be combined to increase 4-HB, 4-HBal, 4-HBCoA, BDO or putrescine or otherwise improve characteristics of the microorganisms of the invention. Each of these non-naturally occurring alterations can result in increased production and an enhanced level of 4-HB, 4-HBal, 4-HBCoA, BDO or putrescine production, for example, during the exponential growth phase of the microbial organism, or otherwise improve the growth or production characteristics of the microorganisms producing 4-HB, 4-HBal, 4-HBCoA, BDO or putrescine compared to a strain that does not contain such metabolic alterations, under appropriate culture conditions. Appropriate conditions include, for example, those disclosed herein, including conditions such as particular carbon sources or reactant availabilities and/or adaptive evolution.

Given the teachings and guidance provided herein, those skilled in the art will understand that to introduce a metabolic alteration such as disruption or attenuation of an enzyme or enzymatic reaction, it can be necessary to disrupt the catalytic activity of the one or more enzymes involved in the reaction. Alternatively, a metabolic alteration can include disruption of expression of a regulatory protein or cofactor necessary for enzyme activity or maximal activity. Furthermore, genetic loss of a cofactor necessary for an enzymatic reaction can also have the same effect as a disruption of the gene encoding the enzyme. Disruption can occur by a variety of methods including, for example, deletion of an encoding gene or incorporation of a genetic alteration in one or more of the encoding gene sequences. The encoding genes targeted for disruption can be one, some, or all of the genes encoding enzymes involved in the catalytic activity. For example, where a single enzyme is involved in a targeted catalytic activity, disruption can occur by a genetic alteration that reduces or eliminates the catalytic activity of the encoded gene product. Similarly, where the single enzyme is multimeric, including heteromeric, disruption can occur by a genetic alteration that reduces or destroys the function of one or all subunits of the encoded gene products. Destruction of activity can be accomplished by loss of the binding activity of one or more subunits required to form an active complex, by destruction of the catalytic subunit of the multimeric complex or by both. Other functions of multimeric protein association and activity also can be targeted in order to disrupt a metabolic reaction of the invention. Such other functions are well known to those skilled in the art. Similarly, a target enzyme activity can be reduced or eliminated by disrupting expression of a protein or enzyme that modifies and/or activates the target enzyme, for example, a molecule required to convert an apoenzyme to a holoenzyme. Further, some or all of the functions of a single polypeptide or multimeric complex can be disrupted according to the invention in order to reduce or abolish the catalytic activity of one or more enzymes involved in a reaction or metabolic modification of the invention. Similarly, some or all of enzymes involved in a reaction or metabolic modification of the invention can be disrupted so long as the targeted reaction is reduced or eliminated.

Given the teachings and guidance provided herein, those skilled in the art also will understand that an enzymatic reaction can be disrupted by reducing or eliminating reactions encoded by a common gene and/or by one or more orthologs of that gene exhibiting similar or substantially the same activity. Reduction of both the common gene and all orthologs can lead to complete abolishment of any catalytic activity of a targeted reaction. However, disruption of either the common gene or one or more orthologs can lead to a reduction in the catalytic activity of the targeted reaction sufficient to promote coupling of growth to product biosynthesis. Exemplified herein are both the common genes encoding catalytic activities for a variety of metabolic modifications as well as their orthologs. Those skilled in the art will understand that disruption of some or all of the genes encoding a enzyme of a targeted metabolic reaction can be practiced in the methods of the invention and incorporated into the non-naturally occurring microbial organisms of the invention in order to achieve the increased production of 4-HB, 4-HBal, 4-HBCoA, BDO or putrescine or growth-coupled product production.

Given the teachings and guidance provided herein, those skilled in the art also will understand that enzymatic activity or expression can be attenuated using well known methods. Reduction of the activity or amount of an enzyme can mimic complete disruption of a gene if the reduction causes activity of the enzyme to fall below a critical level that is normally required for a pathway to function. Reduction of enzymatic activity by various techniques rather than use of gene disruption can be important for an organism's viability. Methods of reducing enzymatic activity that result in similar or identical effects of a gene disruption include, but are not limited to: reducing gene transcription or translation; destabilizing mRNA, protein or catalytic RNA; and mutating a gene that affects enzyme activity or kinetics (see Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999). Natural or imposed regulatory controls can also accomplish enzyme attenuation including: promoter replacement (see Wang et al., Mol. Biotechnol. 52(2):300-308 (2012)); loss or alteration of transcription factors (Dietrick et al., Annu. Rev Biochem. 79:563-590 (2010); and Simicevic et al., Mol. Biosyst. 6(3):462-468 (2010)); introduction of inhibitory RNAs or peptides such as siRNA, antisense RNA, RNA or peptide/small-molecule binding aptamers, ribozymes, aptazymes and riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); and addition of drugs or other chemicals that reduce or disrupt enzymatic activity such as splicing an enzyme inhibitor, an antibiotic or a target-specific drug.

One skilled in the art will also understand and recognize that attenuation of an enzyme can be done at various levels. For example, at the gene level, a mutation causing a partial or complete null phenotype, such as a gene disruption or a mutation causing epistatic genetic effects that mask the activity of a gene product (Miko, Nature Education 1(1)(2008)) can be used to attenuate an enzyme. At the gene expression level, methods for attenuation include: coupling transcription to an endogenous or exogenous inducer such as isopropylthio-β-galactoside (IPTG), then adding low amounts of inducer or no inducer during the production phase (Donovan et al., J. Ind. Microbiol. 16(3):145-154 (1996); and Hansen et al., Curr. Microbiol. 36(6):341-347 (1998)); introducing or modifying a positive or a negative regulator of a gene; modify histone acetylation/deacetylation in a eukaryotic chromosomal region where a gene is integrated (Yang et al., Curr. Opin. Genet. Dev. 13(2):143-153 (2003) and Kurdistani et al., Nat. Rev. Mol. Cell Biol. 4(4):276-284 (2003)); introducing a transposition to disrupt a promoter or a regulatory gene (Bleykasten-Brosshans et al., C. R. Biol. 33(8-9):679-686 (2011); and McCue et al., PLoS Genet. 8(2):e1002474 (2012)); flipping the orientation of a transposable element or promoter region so as to modulate gene expression of an adjacent gene (Wang et al., Genetics 120(4):875-885 (1988); Hayes, Annu. Rev. Genet. 37:3-29 (2003); in a diploid organism, deleting one allele resulting in loss of heterozygosity (Daigaku et al., Mutation Research/Fundamental and Molecular Mechanisms of Mutagenesis 600(1-2)177-183 (2006)); introducing nucleic acids that increase RNA degradation (Houseley et al., Cell 136(4):763-776 (2009)); or in bacteria, for example, introduction of a transfer-messenger RNA (tmRNA) tag, which can lead to RNA degradation and ribosomal stalling (Sunohara et al., RNA 10(3):378-386 (2004); and Sunohara et al., J. Biol. Chem. 279:15368-15375 (2004)). At the translational level, attenuation can include: introducing rare codons to limit translation (Angov, Biotechnol. J. 6(6):650-659 (2011)); introducing RNA interference molecules that block translation (Castel et al., Nat. Rev. Genet. 14(2):100-112 (2013); and Kawasaki et al., Curr. Opin. Mol. Ther. 7(2):125-131 (2005)); modifying regions outside the coding sequence, such as introducing secondary structure into an untranslated region (UTR) to block translation or reduce efficiency of translation (Ringner et al., PLoS Comput. Biol. 1(7):e72 (2005)); adding RNAase sites for rapid transcript degradation (Pasquinelli, Nat. Rev. Genet. 13(4):271-282 (2012); and Arraiano et al., FEMS Microbiol. Rev. 34(5):883-932(2010)); introducing antisense RNA oligomers or antisense transcripts (Nashizawa et al., Front. Biosci. 17:938-958 (2012)); introducing RNA or peptide aptamers, ribozymes, aptazymes, riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); or introducing translational regulatory elements involving RNA structure that can prevent or reduce translation that can be controlled by the presence or absence of small molecules (Araujo et al., Comparative and Functional Genomics, Article ID 475731, 8 pages (2012)). At the level of enzyme localization and/or longevity, enzyme attenuation can include: adding a degradation tag for faster protein turnover (Hochstrasser, Annual Rev. Genet. 30:405-439 (1996); and Yuan et al., PLoS One 8(4):e62529 (2013)); or adding a localization tag that results in the enzyme being secreted or localized to a subcellular compartment in a eukaryotic cell where the enzyme would not be able to react with its normal substrate (Nakai et al. Genomics 14(4):897-911 (1992); and Russell et al., J. Bact. 189(21)7581-7585 (2007)). At the level of post-translational regulation, enzyme attenuation can include: increasing intracellular concentration of known inhibitors; or modifying post-translational modified sites (Mann et al., Nature Biotech. 21:255-261(2003)). At the level of enzyme activity, enzyme attenuation can include: adding an endogenous or an exogenous inhibitor, such as an enzyme inhibitor, an antibiotic, or a target-specific drug, to reduce enzyme activity; limiting availability of essential cofactors, such as vitamin B12, for an enzyme that requires the cofactor; chelating a metal ion that is required for enzyme activity; or introducing a dominant negative mutation. The applicability of a technique for attenuation described above can depend upon whether a given host microbial organism is prokaryotic or eukaryotic, and it is understand that a determination of what is the appropriate technique for a given host can be readily made by one skilled in the art.

In some embodiments, microaerobic designs can be used based on the growth-coupled formation of the desired product. To examine this, production cones can be constructed for each strategy by first maximizing and, subsequently minimizing the product yields at different rates of biomass formation feasible in the network. If the rightmost boundary of all possible phenotypes of the mutant network is a single point, it implies that there is a unique optimum yield of the product at the maximum biomass formation rate possible in the network. In other cases, the rightmost boundary of the feasible phenotypes is a vertical line, indicating that at the point of maximum biomass the network can make any amount of the product in the calculated range, including the lowest amount at the bottommost point of the vertical line. Such designs are given a low priority.

The 4-HB, 4-HBal, 4-HBCoA, BDO or putrescine production strategies identified by the methods disclosed herein such as the OptKnock framework are generally ranked on the basis of their (i) theoretical yields, and optionally (ii) growth-coupled 4-HB, 4-HBal, 4-HBCoA, BDO or putrescine formation characteristics.

Accordingly, the invention also provides a non-naturally occurring microbial organism having a set of metabolic modifications that increase the yield of 4-HB, 4-HBal, 4-HBCoA, BDO or putrescine, optionally coupling 4-HB, 4-HBal, 4-HBCoA, BDO or putrescine production to growth of the organism, or otherwise improve characteristics of the microorganisms producing 4-HB, 4-HBal, 4-HBCoA, BDO or putrescine, where the set of metabolic modifications includes disruption of one or more genes, as described herein.

Each of the strains can be supplemented with additional deletions if it is determined that the strain designs do not sufficiently increase the production of 4-HB, 4-HBal, 4-HBCoA, BDO or putrescine and/or couple the formation of the product with biomass formation. Alternatively, some other enzymes not known to possess significant activity under the growth conditions can become active due to adaptive evolution or random mutagenesis. Such activities can also be knocked out. However, the list of gene deletion sets disclosed herein allows the construction of strains exhibiting high-yield production of 4-HB, 4-HBal, 4-HBCoA, BDO or putrescine, including growth-coupled production of 4-HB, 4-HBal, 4-HBCoA, BDO or putrescine. [00386]4-HB, 4-HBal, 4-HBCoA, BDO or putrescine can be harvested or isolated at any time point during the culturing of the microbial organism, for example, in a continuous and/or near-continuous culture period, as disclosed herein. Generally, the longer the microorganisms are maintained in a continuous and/or near-continuous growth phase, the proportionally greater amount of 4-HB, 4-HBal, 4-HBCoA, BDO or putrescine can be produced.

Therefore, the invention additionally provides a method for producing 4-HB, 4-HBal, 4-HBCoA, BDO or putrescine that includes culturing a non-naturally occurring microbial organism having one or more gene disruptions, as disclosed herein. The disruptions can occur in one or more genes encoding an enzyme that increases production of 4-HB, 4-HBal, 4-HBCoA, BDO or putrescine, including optionally coupling 4-HB, 4-HBal, 4-HBCoA, BDO or putrescine production to growth of the microorganism when the gene disruption reduces or eliminates an activity of the enzyme. For example, the disruptions can confer stable growth-coupled production of 4-HB, 4-HBal, 4-HBCoA, BDO or putrescine onto the non-naturally occurring microbial organism.

In some embodiments, the gene disruption can include a complete gene deletion. In some embodiments other methods to disrupt a gene include, for example, frameshifting by omission or addition of oligonucleotides or by mutations that render the gene inoperable. One skilled in the art will recognize the advantages of gene deletions, however, because of the stability it confers to the non-naturally occurring organism from reverting to a parental phenotype in which the gene disruption has not occurred. In particular, the gene disruptions are selected from the gene sets as disclosed herein.

Once computational or other predictions are made of one or more genes sets disruption to increase production of 4-HB, 4-HBal, 4-HBCoA, BDO or putrescine, the strains can be constructed, evolved, and tested. Gene disruptions, including gene deletions, are introduced into host organism by methods well known in the art. A particularly useful method for gene disruption is by homologous recombination, as disclosed herein.

The engineered strains can be characterized by measuring the growth rate, the substrate uptake rate, and/or the product/byproduct secretion rate. Cultures can be grown and used as inoculum for a fresh batch culture for which measurements are taken during exponential growth. The growth rate can be determined by measuring optical density using a spectrophotometer (A600). Concentrations of glucose and other organic acid byproducts in the culture supernatant can be determined by well known methods such as HPLC, GC-MS or other well known analytical methods suitable for the analysis of the desired product, as disclosed herein, and used to calculate uptake and secretion rates.

Strains containing gene disruptions can exhibit suboptimal growth rates until their metabolic networks have adjusted to their missing functionalities. To assist in this adjustment, the strains can be adaptively evolved. By subjecting the strains to adaptive evolution, cellular growth rate becomes the primary selection pressure and the mutant cells are compelled to reallocate their metabolic fluxes in order to enhance their rates of growth. This reprogramming of metabolism has been recently demonstrated for several E. coli mutants that had been adaptively evolved on various substrates to reach the growth rates predicted apriori by an in silico model (Fong and Palsson, Nat. Genet. 36:1056-1058 (2004)). The growth improvements brought about by adaptive evolution can be accompanied by enhanced rates of 4-HB, 4-HBal, 4-HBCoA, BDO or putrescine production. The strains can optionally be adaptively evolved in replicate, running in parallel, to account for differences in the evolutionary patterns that can be exhibited by a host organism (Fong and Palsson, Nat. Genet. 36:1056-1058 (2004); Fong et al., J. Bacteriol. 185:6400-6408 (2003); Ibarra et al., Nature 420:186-189 (2002)) that could potentially result in one strain having superior production qualities over the others. Evolutions can be run for a period of time, typically 2-6 weeks, depending upon the rate of growth improvement attained. In general, evolutions are stopped once a stable phenotype is obtained.

Following the adaptive evolution process, the new strains are characterized again by measuring the growth rate, the substrate uptake rate, and the product/byproduct secretion rate. These results are compared to the theoretical predictions by plotting actual growth and production yields along side the production envelopes from metabolic modeling. The most successful design/evolution combinations are chosen to pursue further, and are characterized in lab-scale batch and continuous fermentations. The growth-coupled biochemical production concept behind the methods disclosed herein such as OptKnock approach should also result in the generation of genetically stable overproducers. Thus, the cultures are maintained in continuous mode for an extended period of time, for example, one month or more, to evaluate long-term stability. Periodic samples can be taken to ensure that yield and productivity are maintained.

Adaptive evolution is a powerful technique that can be used to increase growth rates of mutant or engineered microbial strains, or of wild-type strains growing under unnatural environmental conditions. It is especially useful for strains designed via methods such as OptKnock, which results in growth-coupled product formation. Therefore, evolution toward optimal growing strains will indirectly optimize production as well. Unique strains of E. coli K-12 MG1655 were created through gene knockouts and adaptive evolution. (Fong and Palsson, Nat. Genet. 36:1056-1058 (2004)). In this work, all adaptive evolutionary cultures were maintained in prolonged exponential growth by serial passage of batch cultures into fresh medium before the stationary phase was reached, thus rendering growth rate as the primary selection pressure. Knockout strains were constructed and evolved on minimal medium supplemented with different carbon substrates (four for each knockout strain). Evolution cultures were carried out in duplicate or triplicate, giving a total of 50 evolution knockout strains. The evolution cultures were maintained in exponential growth until a stable growth rate was reached. The computational predictions were accurate (within 10%) at predicting the post-evolution growth rate of the knockout strains in 38 out of the 50 cases examined. Furthermore, a combination of OptKnock design with adaptive evolution has led to improved lactic acid production strains. (Fong et al., Biotechnol. Bioeng. 91:643-648 (2005)). Similar methods can be applied to the strains disclosed herein and applied to various host strains.

There are a number of developed technologies for carrying out adaptive evolution. Exemplary methods are disclosed herein. In some embodiments, optimization of a non-naturally occurring organism of the present invention includes utilizing adaptive evolution techniques to increase 4-HB, 4-HBal, 4-HBCoA, BDO or putrescine production and/or stability of the producing strain.

Serial culture involves repetitive transfer of a small volume of grown culture to a much larger vessel containing fresh growth medium. When the cultured organisms have grown to saturation in the new vessel, the process is repeated. This method has been used to achieve the longest demonstrations of sustained culture in the literature (Lenski and Travisano, Proc. Natl. Acad. Sci. USA 91:6808-6814 (1994)) in experiments which clearly demonstrated consistent improvement in reproductive rate over a period of years. Typically, transfer of cultures is usually performed during exponential phase, so each day the transfer volume is precisely calculated to maintain exponential growth through the next 24 hour period. Manual serial dilution is inexpensive and easy to parallelize.

In continuous culture the growth of cells in a chemostat represents an extreme case of dilution in which a very high fraction of the cell population remains. As a culture grows and becomes saturated, a small proportion of the grown culture is replaced with fresh media, allowing the culture to continually grow at close to its maximum population size. Chemostats have been used to demonstrate short periods of rapid improvement in reproductive rate (Dykhuizen, Methods Enzymol. 613-631(1993)). The potential usefulness of these devices was recognized, but traditional chemostats were unable to sustain long periods of selection for increased reproduction rate, due to the unintended selection of dilution-resistant (static) variants. These variants are able to resist dilution by adhering to the surface of the chemostat, and by doing so, outcompete less adherent individuals, including those that have higher reproductive rates, thus obviating the intended purpose of the device (Chao and Ramsdell, J. Gen. Microbiol 20:132-138 (1985)). One possible way to overcome this drawback is the implementation of a device with two growth chambers, which periodically undergo transient phases of sterilization, as described previously (Marliere and Mutzel, U.S. Pat. No. 6,686,194).

Evolugator™ is a continuous culture device developed by Evolugate, LLC (Gainesville, FL) and exhibits significant time and effort savings over traditional evolution techniques (de Crecy et al., Appl. Microbiol. Biotechnol. 77:489496 (2007)). The cells are maintained in prolonged exponential growth by the serial passage of batch cultures into fresh medium before the stationary phase is attained. By automating optical density measurement and liquid handling, the Evolugator™ can perform serial transfer at high rates using large culture volumes, thus approaching the efficiency of a chemostat in evolution of cell fitness. For example, a mutant of Acinetobacter sp ADP1 deficient in a component of the translation apparatus, and having severely hampered growth, was evolved in 200 generations to 80% of the wild-type growth rate. However, in contrast to the chemostat which maintains cells in a single vessel, the machine operates by moving from one “reactor” to the next in subdivided regions of a spool of tubing, thus eliminating any selection for wall-growth. The transfer volume is adjustable, and normally set to about 50%. A drawback to this device is that it is large and costly, thus running large numbers of evolutions in parallel is not practical. Furthermore, gas addition is not well regulated, and strict anaerobic conditions are not maintained with the current device configuration. Nevertheless, this is an alternative method to adaptively evolve a production strain.

As disclosed herein, a nucleic acid encoding a desired activity of a 4-HB, 4-HBal, 4-HBCoA, BDO or putrescine pathway can be introduced into a host organism. In some cases, it can be desirable to modify an activity of a 4-HB, 4-HBal, 4-HBCoA BDO or putrescine pathway enzyme or protein to increase production of 4-HB, 4-HBal, 4-HBCoA BDO or putrescine. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742(2007), Patel (ed.), CRC Press; Otten and Quax. Biomol. Eng 22:1-9 (2005).; and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of a 4-HB, 4-HBal, 4-HBCoA, BDO or putrescine pathway enzyme or protein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751(1994); and Stemmer, Nature 370:389-391 (1994)); Staggered Extension (StEP), which entails template priming followed by repeated cycles of 2 step PCR with denaturation and very short duration of annealing/extension (as short as 5 sec)(Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681-683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456463 (2000)); Random Chimeragenesis on Transient Templates (RACHITT), which employs Dnase I fragmentation and size fractionation of single stranded DNA (ssDNA)(Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117(2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591(2001)); the Mutator Strains technique, in which conditional ts mutator plasmids, utilizing the mutD5 gene, which encodes a mutant subunit of DNA polymerase I, to allow increases of 20 to 4000-× in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931(2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego CA), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

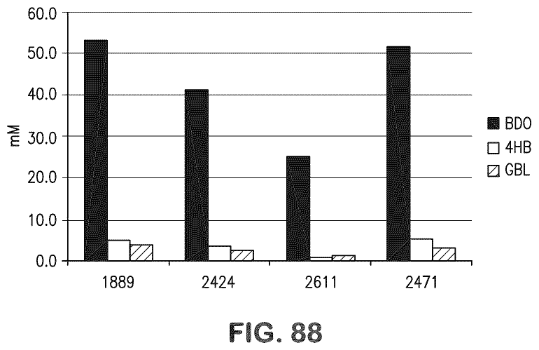

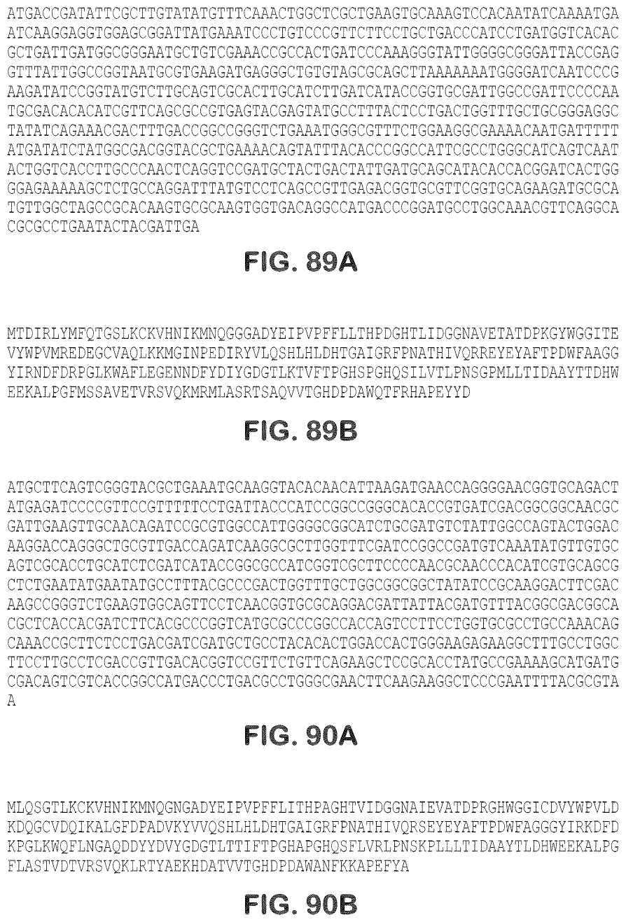

As disclosed herein, esterases were utilized to convert gamma-butyrolactone to 4-hydroxybutyrate. Exemplary esterases include, but are not limited to, esterases from Yersinia intermedia 29909 (locus yinte0001_13710) and Agrobacterium tumefaciens str. C58 (Carlier et al., Mol. Plant Microbe Interact. 17(9):951-957 (2004))(see Example XXXIV). The nucleotide sequence and amino acid sequence of the Yersinia gene (locus yinte0001_13710) is shown in FIG. 89 (see also GenBank ZP_04636075.1; GI:238792441). The nucleotide and amino acid sequence of the gene from Agrobacterium tumefaciens is shown in FIG. 90. Additionally exemplary esterases include AttM of Escherichia coli KTE10 (GenBank ZP_19612688.1, GI:432369596) and attM of Photorhabdus asymbiotica (GenBank YP_003039319.1 GI:253987963).

The invention additionally provides a method for reducing gamma-butyrolactone production in a cell or cell culture by expressing in the cell a gamma-butyrolactone esterase. The gamma-butyrolactone esterase can be, for example, a gamma-butyrolactone esterase of Yersinia or a polypeptide having at least 90% identity to a gamma-butyrolactone esterase of Yersinia. In a particular embodiment, the gamma-butyrolactone esterase is encoded by a nucleic acid encoding the amino acid sequence of SEQ ID NO:, or a nucleic acid encoding a polypeptide having at least 90% identity to SEQ ID NO:. The invention further provides utilizing a gamma-butyrolactone esterase to decrease the amount of gamma-butyrolactone in a sample in vitro.

In some embodiments, the invention provides an isolated nucleic acid molecule selected from: (a) a nucleic acid molecule encoding an amino acid sequence disclosed herein, such as those referenced in Tables 53, 55 or 57 or Example XXXXI, wherein the amino acid sequence comprises one or more variant amino acid positions disclosed herein, such as those set forth in Tables 54, 56 or 58 or Example XXXXIII; (b) a nucleic acid molecule that hybridizes to the nucleic acid of (a) under highly stringent hybridization conditions and comprises a nucleic acid sequence that encodes one or more variant amino acid positions disclosed herein, such as those set forth in Tables 54, 56 or 58 or Example XXXXIII; and a nucleic acid molecule that is complementary to (a) or (b). In some aspects, the isolated nucleic acid molecule can have at least 65%, 70%, 75%, 80%, 85%, 90%, 91%, 92%, 93%, 94%, 95%, 96%, 97%, 98%, or 99% sequence identity, or is identical (100% identity), to an amino acids sequence disclosed herein, such as those referenced in Tables 53, 55 or 57 or Example XXXXIII.

It is understood that a nucleic acid of the invention can exclude a wild type parental sequence, for example a parental sequence as disclosed in any of Tables 53-58. One skilled in the art will readily understand the meaning of a parental wild type sequence based on what is well known in the art. It is further understood that such a nucleic acid can exclude a naturally occurring amino acid sequence as found in nature. Thus, in a particular embodiment, the nucleic acid of the invention is as set forth above and herein, with the proviso that the encoded amino acid sequence is not the wild type parental sequence or a naturally occurring amino acid sequence and/or that the nucleic acid sequence is not a wild type or naturally occurring nucleic acid sequence. A naturally occurring amino acid or nucleic acid sequence is understood by those skilled in the art as relating to a sequence that is found in a naturally occurring organism. Thus, a nucleic acid or amino acid sequence that is not found in the same state or having the same nucleotide or encoded amino acid sequence as in a naturally occurring organism is included within the meaning of a nucleic acid and/or amino acid sequence of the invention. For example, a nucleic acid or amino acid sequence that has been altered at one or more nucleotide or amino acid positions from a parent sequence, including variants as described herein (Tables 53-58 and Example XXXXII), are included within the meaning of a nucleic acid or amino acid sequence of the invention that is not naturally occurring. An isolated nucleic acid molecule of the invention excludes a naturally occurring chromosome that contains the nucleic acid sequence, and can further exclude other molecules as found in a naturally occurring cell such as DNA binding proteins, for example, proteins such as histones that bind to chromosomes with a eukaryotic cell.

Thus, an isolated nucleic acid sequence of the invention has physical and chemical differences compared to a naturally occurring nucleic acid sequence. An isolated or non-naturally occurring nucleic acid of the invention does not contain or does not necessarily have some or all of the chemical bonds, either covalent or non-covalent bonds, of a naturally occurring nucleic acid sequence as found in nature. An isolated nucleic acid of the invention thus differs from a naturally occurring nucleic acid, for example, by having a different chemical structure than a naturally occurring nucleic acid sequence as found in a chromosome. A different chemical structure can occur, for example, by cleavage of phosphodiester bonds that release an isolated nucleic acid sequence from a naturally occurring chromosome. An isolated nucleic acid of the invention can also differ from a naturally occurring nucleic acid by isolating or separating the nucleic acid from proteins that bind to chromosomal DNA in either prokaryotic or eukaryotic cells, thereby differing from a naturally occurring nucleic acid by different non-covalent bonds. With respect to nucleic acids of prokaryotic origin, a non-naturally occurring nucleic acid of the invention does not necessarily have some or all of the naturally occurring chemical bonds of a chromosome, for example, binding to DNA binding proteins such as polymerases or chromosome structural proteins, or is not in a higher order structure such as being supercoiled. With respect to nucleic acids of eukaryotic origin, a non-naturally occurring nucleic acid of the invention also does not contain the same internal nucleic acid chemical bonds or chemical bonds with structural proteins as found in chromatin. For example, a non-naturally occurring nucleic acid of the invention is not chemically bonded to histones or scaffold proteins and is not contained in a centromere or telomere. Thus, the non-naturally occurring nucleic acids of the invention are chemically distinct from a naturally occurring nucleic acid because they either lack or contain different van der Waals interactions, hydrogen bonds, ionic or electrostatic bonds, and/or covalent bonds from a nucleic acid as found in nature. Such differences in bonds can occur either internally within separate regions of the nucleic acid (that is cis) or such difference in bonds can occur in trans, for example, interactions with chromosomal proteins. In the case of a nucleic acid of eukaryotic origin, a cDNA is considered to be an isolated or non-naturally occurring nucleic acid since the chemical bonds within a cDNA differ from the covalent bonds, that is the sequence, of a gene on chromosomal DNA. Thus, it is understood by those skilled in the art that an isolated or non-naturally occurring nucleic acid is distinct from a naturally occurring nucleic acid.

A nucleic acid molecule encoding a 4-HB, GBL, 4-HBal, 4-HBCoA, BDO and/or putrescine pathway enzyme or protein of the invention can also include a nucleic acid molecule that hybridizes to a nucleic acid disclosed herein by SEQ ID NO, GenBank and/or GI number or a nucleic acid molecule that hybridizes to a nucleic acid molecule that encodes an amino acid sequence disclosed herein by SEQ ID NO, GenBank and/or GI number. Hybridization conditions can include highly stringent, moderately stringent, or low stringency hybridization conditions that are well known to one of skill in the art such as those described herein. Similarly, a nucleic acid molecule that can be used in the invention can be described as having a certain percent sequence identity to a nucleic acid disclosed herein by SEQ ID NO, GenBank and/or GI number or a nucleic acid molecule that hybridizes to a nucleic acid molecule that encodes an amino acid sequence disclosed herein by SEQ ID NO, GenBank and/or GI number. For example, the nucleic acid molecule can have at least 65%, 70%, 75%, 80%, 85%, 90%, 91%, 92%, 93%, 94%, 95%, 96%, 97%, 98% or 99% sequence identity, or is identical, to a nucleic acid described herein.

The phrase “stringent hybridization” is used herein to refer to conditions under which hybridized polynucleotides are stable. As known to those of skill in the art, the stability of hybridized polynucleotides is reflected in the melting temperature (Tₘ) of the hybrids. In general, the stability of a polynucleotide hybrid is a function of the salt concentration, for example, sodium ion concentration and temperature. A hybridization reaction can be performed under conditions of lower stringency, followed by washes of varying, but higher, stringency. Reference to hybridization stringency relates to such washing conditions.

The phrase “highly stringent hybridization” refers to conditions that permit hybridization of only those nucleic acid sequences that form stable hybridized polynucleotides in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5×Denhart's solution, 5×SSPE, 0.2% SDS at 42° C., followed by washing in 0.1×SSPE, and 0.1% SDS at 65° C.

Hybridization conditions other than highly stringent hybridization conditions can be used to describe the nucleic acid sequences disclosed herein. For example, the phrase moderately stringent hybridization refers to conditions equivalent to hybridization in 50% formamide, 5×Denhart's solution, 5×SSPE, 0.2% SDS at 42° C., followed by washing in 0.2×SSPE, 0.2% SDS, at 42° C. The phrase low stringency hybridization refers to conditions equivalent to hybridization in 100 formamide, 5×Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA).

Other suitable low, moderate and high stringency hybridization buffers and conditions are well known to those of skill in the art and are described, for example, in Sambrook et al., Molecular Cloning: A Laboratory Manual, Cold Spring Harbor Laboratories, Cold Spring Harbor, N.Y., 1989; and Ausubel et al., Current Protocols in Molecular Biology, John Wiley and Sons, Baltimore, MD (1999)). Nucleic acids encoding polypeptides of the invention that hybridize under moderately stringent or high stringency conditions to substantially the entire sequence, or a substantial portion, for example, can be at least 15-30 nucleotides of the nucleic acid sequence set forth herein.

In certain embodiments, the isolated nucleic acid molecule of the invention encodes an amino acid sequence that includes at least two, three, four, five, six, seven, eight, nine, ten, eleven, twelve, thirteen, fourteen, fifteen, sixteen, seventeen, eighteen, nineteen, twenty or more variants in any combination disclosed herein. The variants can include any combination of those set forth in Tables 54, 56 or 58 or Example XXXXIII. The variants alone or in combination can produce an enzyme that retains or improves the activity of the wild-type (native) enzyme encoded by the nucleic acid molecule. Methods of generating and assaying such variant are well known to one of skill in the art.

In certain aspects, the invention provides a nucleic acid molecule that encodes an amino acid sequence having one or more variant disclosed herein, wherein the nucleic acid molecule has at least a certain sequence identity to a nucleotide sequence disclosed herein, for example, a nucleic acid molecule encoding a 4-HB, GBL, 4-HBal, 4-HBCoA, BDO and/or putrescine pathway enzyme or protein of the invention, can have at least a certain identity to a nucleotide sequence disclosed herein. Accordingly, in some embodiments, the invention provides a nucleic acid molecule having a nucleotide sequence of at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleotide sequence disclosed herein, for example, by sequence, SEQ ID NO, GenBank and/or GI number, or a nucleic acid molecule that hybridizes to a nucleic acid molecule that encodes an amino acid sequence disclosed herein by sequence, SEQ ID NO, GenBank and/or GI number. The invention still further provides a nucleic acid molecule that encodes an amino acid sequence having one or more variant amino acid positions as disclosed herein, wherein the amino acid sequence has at least 65% identity, at least 70% identity, at least 75% identity, at least 80% identity, at least 85% identity, at least 90% identity, at least 91%, at least 92%, at least 93%, at least 94%, at least 95% identity, at least 96%, at least 97%, at least 98% identity, or at least 990 identity, or is identical, to an amino acid sequence disclosed herein. It is understood that the nucleic acids of the invention, including those with recited % identity, that encode variant polypeptides such as cat2, aldehyde dehydrogenase and alcohol dehydrogenase encode variant polypeptides that can cany out a similar enzymatic reaction as the parent polypeptide, for example, converting 4-hydroxybutyrate to 4-hydroxybutyryl-CoA, converting 4-hydroxybutyryl-CoA to 4-hydroxybutyraldehyde, or converting 4-hydroxybutyraldehyde to 1,4-butanediol, as disclosed herein (see Example XXXXII).

Accordingly, in some aspects of the invention, a nucleic acid molecule encoding a 4-HB, GBL, 4-HBal, 4-HBCoA, BDO and/or putrescine pathway enzyme or protein has a nucleotide sequence of at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleic acid disclosed herein by SEQ ID NO, GenBank and/or GI number or a nucleic acid molecule that hybridizes to a nucleic acid molecule that encodes an amino acid sequence disclosed herein by SEQ ID NO, GenBank and/or GI number.

In some embodiments, the invention provides a vector containing a nucleic acid molecule disclosed herein. In some aspects, the vector can be an expression vector as disclosed herein.

In some embodiments, the invention provides a host cell containing a vector disclosed herein. In some aspects, the host cell can be a non-naturally occurring microbial organism having a pathway that produces 4-HB, GBL, 4-HBal, 4-HBCoA, BDO and/or putrescine. The nucleic acid molecule contained in the host cell can be expressed using methods well known to one of skill in the art, such as using a promoter and/or enhancer that allows expression of the nucleic acid molecule in a desired host cell. Any of a variety of inducible promoters or enhancers can also be included in the vector for regulated expression of a nucleic acid molecule.

In certain embodiments, the invention provides a host cell disclosed herein, wherein the nucleic acid molecule of the invention is integrated into the host chromosome. Integration of the nucleic acid molecule can be done using methods well known to one of skill in the art, such as those disclosed herein. For example, the methods used to integrate nucleic acids into a host chromosome described herein is not limited to the precise nucleic acid molecule integrated. It is understood that one of skill in the art would be able to apply these same methods for integration of desired nucleic acids at the same or alternative sites within the genome of a host cell. In some aspects, integration of the nucleic acid molecule of the invention can be site-specific. In some aspects, the site specific integration will be of a minimal amount of the nucleic acid molecule such as the coding region of the desired polypeptide, which can be expressed by an endogenous host promoter. Methods of site-specific integration are well known to those of skill in the art, any number of which can be used to generate the host cells of the invention. Alternatively, the nucleic acid molecule can be expressed in a cell where the nucleic acid molecule is not integrated into a host chromosome, for example, expressed in a cell from a plasmid or other vector, as disclosed herein.

In some embodiments, the host cell of the invention is a species of microorganism that is capable of fermentation. Exemplary microorganisms that are capable of fermentation are described herein. Furthermore, in some embodiments, the invention provides culture medium containing one or more host cells of the invention. In some aspect, the culture medium can be purified or substantially purified from a host cell of the invention following culturing of the host cell for production of 4-HB, GBL, 4-HBal, 4-HBCoA, BDO and/or putrescine. Methods of purifying or substantially purifying culture medium are well known to one skilled in the art and any one of which can be used to generate the culture medium of the invention, including those methods disclosed herein.

In some embodiments, the invention provides a host strain, a method of constructing a host strain, or a method of fermentation to produce 4-HB, GBL, 4-HBal, 4-HBCoA, BDO and/or putrescine with the host strain, wherein the host strain includes a deletion and/or modification that attenuates expression or activity of one or more, up to all, native host genes encoding a polypeptide (enzyme) that catalyzes the same reaction in a 4-HB, GBL, 4-HBal, 4-HBCoA, BDO and/or putrescine pathway disclosed herein. In some aspects, the host strain provides an improved yield of 4-HB, GBL, 4-HBal, 4-HBCoA, BDO and/or putrescine in the fermentation compared to the parent strain before the deletion and/or modification.

In some embodiments, the invention provides an isolated polypeptide having an amino acid sequence disclosed herein, such as those referenced in Tables 53, 55 or 57 or Example XXXXII, wherein the amino acid sequence includes one or more variant amino acid positions as set forth in Tables 54, 56 or 58 or Example XXXXIII. In some aspects, the isolated polypeptide of the invention includes an amino acid sequence, other than the one or more variant amino acid positions as set forth in Tables 54, 56 or 58 or Example XXXXII, with at least 65%, 70%, 75%, 80%, 85%, 90%, 91%, 92%, 93%, 94%, 95%, 96%, 97%, 98% or 99% sequence identity, or is identical, to an amino acids sequence referenced in Tables 54, 56 or 58 or Example XXXXIII. It is understood that a variant amino acid position can include any one of the 20 naturally occurring amino acids, a conservative substitution of a wild type or parental sequence at the corresponding position of the variant amino acid position, or a specific amino acid at the variant amino acid position such as those disclosed herein in Example XXXXIII. It is further understood that any of the variant amino acid positions can be combined to generate further variants. Variants with combinations of two or more variant amino acid positions exhibited activities greater than wild type (see Example XXXXIII). Thus, as exemplified herein, generating enzyme variants by combining active variant amino acid positions resulted in enzyme variants with improved properties (see Example XXXXII). One skilled in the art can readily generate polypeptides with single variant positions or combinations of variant positions using methods well known to those skilled in the art to generate polypeptides with desired properties, including increased enzyme activity and/or stability.

“Homology” or “identity” or “similarity” refers to sequence similarity between two polypeptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

A polypeptide or polypeptide region (or a polynucleotide or polynucleotide region) has a certain percentage (for example, 65%, 70%, 75%, 80%, 85%, 90%, 95%, 98% or 99%) of “sequence identity” to another sequence means that, when aligned, that percentage of amino acids (or nucleotide bases) are the same in comparing the two sequences. Sequence identity (also known as homology or similarity) refers to sequence similarity between two nucleic acid molecules or between two polypeptides. Identity can be determined by comparing a position in each sequence, which may be aligned for purposes of comparison. When a position in the compared sequence is occupied by the same base or amino acid, then the molecules are identical at that position. A degree of identity between sequences is a function of the number of matching or homologous positions shared by the sequences. This alignment and the percent homology or sequence identity can be determined using software programs known in the art, for example those described in Ausubel et al., supra. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information (NCBI).

In certain embodiments, the invention provides an isolated polypeptide having an amino acid sequence that includes at least two, three, four, five, six, seven, eight, nine, ten, eleven, twelve, thirteen, fourteen, fifteen, sixteen, seventeen, eighteen, nineteen, twenty or more variants in any combination disclosed herein. The variants can include any combination of the variants set forth in Tables 54, 56 or 58 or Example XXXXIII. The variants alone or in combination can produce an enzyme that retains or improves the activity of the wild-type (native) enzyme encoded by the nucleic acid molecule. In some aspects, the polypeptide of the invention having any combination of variants set forth in Table 54 or Example XXXXIII (4-hydroxybutyrate coenzyme A transferase; cat 2) can convert 4-hydroxybutyrate to 4-hydroxybutyryl-CoA. In some aspects, the polypeptide of the invention having any combination of variants set forth in Table 58 or Example XXXXIII aldehyde dehydrogenase) can convert 4-hydroxybutyryl-CoA to 4-hydroxybutyraldehyde. In some aspects, the polypeptide of the invention having any combination of variants set forth in Table 56 or Example XXXXIII (alcohol dehydrogenase) can convert 4-hydroxybutyraldehyde to 1,4-butanediol. Methods of generating and assaying such polypeptides are well known to one of skill in the art.

In some embodiments, the isolated polypeptide of the invention can further include a conservative amino acid substitution in from 1 to 100 amino acid positions, or alternatively from 2 to 100 amino acid positions, or alternatively from 3 to 100 amino acid positions, or alternatively from 4 to 100 amino acid positions, or alternatively from 5 to 100 amino acid positions, or alternatively from 6 to 100 amino acid positions, or alternatively from 7 to 100 amino acid positions, or alternatively from 8 to 100 amino acid positions, or alternatively from 9 to 100 amino acid positions, or alternatively from 10 to 100 amino acid positions, or alternatively from 15 to 100 amino acid positions, or alternatively from 20 to 100 amino acid positions, or alternatively from 30 to 100 amino acid positions, or alternatively from 40 to 100 amino acid positions, or alternatively from 50 to 100 amino acid positions, or any integer therein, wherein the positions are other than the variant amino acid positions set forth in Tables 54, 56 or 58 or Example XXXXIII. In some aspects, the conservative amino acid sequence is a chemically conservative or an evolutionary conservative amino acid substitution. Methods of identifying conservative amino acids are well known to one of skill in the art, any one of which can be used to generate the isolated polypeptides of the invention.

In some embodiments, the isolated polypeptide of the invention can include no modification at from 2 to 300 amino acid positions, or alternatively 2 to 300 amino acid positions, or alternatively 3 to 300 amino acid positions, or alternatively 4 to 300 amino acid positions, or alternatively 5 to 300 amino acid positions, or alternatively 10 to 300 amino acid positions, or alternatively 20 to 300 amino acid positions, or alternatively 30 to 300 amino acid positions, or alternatively 40 to 300 amino acid positions, or alternatively 50 to 300 amino acid positions, or alternatively 60 to 300 amino acid positions, or alternatively 80 to 300 amino acid positions, or alternatively 100 to 300 amino acid positions, or alternatively 150 to 300 amino acid positions, or alternatively 200 to 300 amino acid positions, or alternatively 250 to 300 amino acid positions, or any integer therein, compared to the parent (wild-type) sequence, wherein the positions are selected from those that are identical to between 2, 3, 4, or 5 of the amino acid sequences referenced in Tables 53, 55 or 57. In a particular embodiment, a variant polypeptide of the invention that is a variant aldehyde dehydrogenase includes a variant aldehyde dehydrogenase as disclosed herein, with the proviso that the variant aldehyde dehydrogenase excludes the substitution Ile139Leu, Met204Asp, and/or Arg396Lys (numbered according to the sequence described in US 2014/0045232).

It is understood that the variant polypeptides such as polypeptide variants of cat2, aldehyde dehydrogenase and alcohol dehydrogenase can cany out a similar enzymatic reaction as the parent polypeptide, for example, converting 4-hydroxybutyrate to 4-hydroxybutyryl-CoA, converting 4-hydroxybutyryl-CoA to 4-hydroxybutyraldehyde, or converting 4-hydroxybutyraldehyde to 1,4-butanediol, as disclosed herein (see Example XXXXII). It is further understood that the polypeptide variants can include variants that provide a beneficial characteristic to the polypeptide, including but not limited to, improved catalytic activity, increased catalytic, turnover, increased substrate affinity, decreased product inhibition, and/or protein or enzyme stability. In a particular embodiment, the variants can have improved characteristics of stability while exhibiting similar activity to a wild type or parent polypeptide. In another particular embodiment, the enzyme variant can exhibit an activity that is at least the same or higher than a wild type or parent polypeptide, for example, 1.2, 1.5, 2, 2.5, 3, 3.5, 4, 4.5, 5, 5.5, 6, 6.5, 7, 7.5, 8, 8.5, 9, 9.5, 10, or even higher fold activity of the variant polypeptide over a wild type or parent polypeptide (see Example XXXXII).

The polypeptides of the invention can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present invention can be obtained using well-known recombinant methods (see, for example, Sambrook et al., supra, 1989; Ausubel et al., supra, 1999). The methods and conditions for biochemical purification of a polypeptide of the invention can be chosen by those skilled in the art, and purification monitored, for example, by a functional assay.

One non-limiting example of a method for preparing the invention polypeptide is to express nucleic acids encoding the polypeptide in a suitable host cell, such as a bacterial cell, a yeast cell, or other suitable cell, using methods well known in the art, and recovering the expressed polypeptide, again using well-known purification methods, as described herein. Invention polypeptides can be isolated directly from cells that have been transformed with expression vectors as described herein. Recombinantly expressed polypeptides of the invention can also be expressed as fusion proteins with appropriate affinity tags, such as glutathione S transferase (GST), poly His, streptavidin, and the like, and affinity purified, if desired. A polypeptide of the invention can retain the affinity tag, if desired, or optionally the affinity tag can be removed from the polypeptide using well known methods to remove an affinity tag, for example, using appropriate enzymatic or chemical cleavage. Thus, the invention provides polypeptides of the invention without or optionally with an affinity tag. Accordingly, in some embodiments, the invention provides a host cell expressing a polypeptide of the invention disclosed herein. An invention polypeptide can also be produced by chemical synthesis using a method of polypeptide synthesis well know to one of skill in the art.

In some embodiments, the invention provides using a polypeptide disclosed herein as a biocatalyst. A “biocatalyst,” as used herein, refers to a biological substance that initiates or modifies the rate of a chemical reaction. A biocatalyst can be an enzyme. A polypeptide of the invention can be used to increase the rate of conversion of a substrate to a product as disclosed herein. In the context of an industrial reaction, a polypeptide of the invention can be used, absent a host cell, to improve reactions generating 4-HB, GBL, 4-HBal, 4-HBCoA, BDO and/or putrescine, for example, using in vitro methods.

In some embodiments, the invention provides a composition having a polypeptide disclosed herein and at least one substrate for the polypeptide. Substrate for each of the polypeptides disclosed herein are described herein and are exemplified in the Figures. The polypeptide within the composition of the invention can react with a substrate under in vitro conditions. In this context, an in vitro condition refers to a reaction in the absence of or outside of a microorganism of the invention.

In some embodiments, the invention provides a method of constructing a host strain that can include, among other steps, introducing a vector disclosed herein into a host cell that is capable of fermentation. Vectors of the invention can be introduced stably or transiently into a host cell using techniques well known in the art including, but not limited to, conjugation, electroporation, chemical transformation, transduction, transfection, and ultrasound transformation. Additional methods are disclosed herein, any one of which can be used in the method of the invention.

In some embodiments, the invention provides a method for producing 4-HB, GBL, 4-HBal, 4-HBCoA, BDO and/or putrescine by culturing a host cell disclosed herein under conditions and for a sufficient period of time to produce 4-HB, GBL, 4-HBal, 4-HBCoA, BDO and/or putrescine. Exemplary culturing conditions, including various fermentation conditions, are disclosed herein, any one of which can be used in the method of the invention.

It is understood that modifications which do not substantially affect the activity of the various embodiments of this invention are also provided within the definition of the invention provided herein. Accordingly, the following examples are intended to illustrate but not limit the present invention.

### Example I

### Biosynthesis of 4-Hydroxybutanoic Acid

This example describes exemplary biochemical pathways for 4-HB production.

Previous reports of 4-HB synthesis in microbes have focused on this compound as an intermediate in production of the biodegradable plastic poly-hydroxyalkanoate (PHA)(U.S. Pat. No. 6,117,658). The use of 4-HB/3-HB copolymers over poly-3-hydroxybutyrate polymer (PHB) can result in plastic that is less brittle (Saito and Doi, Intl. J. Biol. Macromol. 16:99-104 (1994)). The production of monomeric 4-HB described herein is a fundamentally distinct process for several reasons: (1) the product is secreted, as opposed to PHA which is produced intracellularly and remains in the cell; (2) for organisms that produce hydroxybutanoate polymers, free 4-HB is not produced, but rather the Coenzyme A derivative is used by the polyhydroxyalkanoate synthase; (3) in the case of the polymer, formation of the granular product changes thermodynamics; and (4) extracellular pH is not an issue for production of the polymer, whereas it will affect whether 4-HB is present in the free acid or conjugate base state, and also the equilibrium between 4-HB and GBL.

4-HB can be produced in two enzymatic reduction steps from succinate, a central metabolite of the TCA cycle, with succinic semialdehyde as the intermediate (FIG. 1). The first of these enzymes, succinic semialdehyde dehydrogenase, is native to many organisms including E. coli, in which both NADH- and NADPH-dependent enzymes have been found (Donnelly and Cooper, Eur. J. Biochem. 113:555-561 (1981); Donnelly and Cooper, J. Bacteriol. 145:1425-1427 (1981); Marek and Henson, J. Bacteriol. 170:991-994 (1988)). There is also evidence supporting succinic semialdehyde dehydrogenase activity in S. cerevisiae (Ramos et al., Eur. J. Biochem. 149:401404 (1985)), and a putative gene has been identified by sequence homology. However, most reports indicate that this enzyme proceeds in the direction of succinate synthesis, as shown in FIG. 1 (Donnelly and Cooper, supra; Lutke-Eversloh and Steinbuchel, FEMS Microbiol. Lett. 181:63-71(1999)), participating in the degradation pathway of 4-HB and gamma-aminobutyrate. Succinic semialdehyde also is natively produced by certain microbial organisms such as E. coli through the TCA cycle intermediate α-ketogluterate via the action of two enzymes: glutamate:succinic semialdehyde transaminase and glutamate decarboxylase. An alternative pathway, used by the obligate anaerobe Clostridium kluyveri to degrade succinate, activates succinate to succinyl-CoA, then converts succinyl-CoA to succinic semialdehyde using an alternative succinic semialdehyde dehydrogenase which is known to function in this direction (Sohling and Gottschalk, Eur. J. Biochem. 212:121-127 (1993)). However, this route has the energetic cost of ATP required to convert succinate to succinyl-CoA.

The second enzyme of the pathway, 4-hydroxybutanoate dehydrogenase, is not native to E. coli or yeast but is found in various bacteria such as C. kluyveri and Ralstonia eutropha (Lutke-Eversloh and Steinbuchel, supra; Sohling and Gottschalk, J. Bacteriol. 178:871-880 (1996); Valentin et al., Eur. J. Biochem. 227:43-60 (1995); Wolff and Kenealy, Protein Expr. Purif. 6:206-212 (1995)). These enzymes are known to be NADH-dependent, though NADPH-dependent forms also exist. An additional pathway to 4-HB from alpha-ketoglutarate was demonstrated in E. coli resulting in the accumulation of poly(4-hydroxybutyric acid) (Song et al., Wei Sheng Wu Xue. Bao. 45:382-386 (2005)). The recombinant strain required the overexpression of three heterologous genes, PHA synthase (R. eutropha), 4-hydroxybutyrate dehydrogenase (R. eutropha) and 4-hydroxybutyrate:CoA transferase (C. kluyveri), along with two native E. coli genes: glutamate:succinic semialdehyde transaminase and glutamate decarboxylase. Steps 4 and 5 in FIG. 1 can alternatively be carried out by an alpha-ketoglutarate decarboxylase such as the one identified in Euglena gracilis (Shigeoka et al., Biochem. J. 282(Pt2):319-323 (1992); Shigeoka and Nakano, Arch. Biochem. Biophys. 288:22-28 (1991); Shigeoka and Nakano, Biochem J. 292(Pt 2):463-467 (1993)). However, this enzyme has not previously been applied to impact the production of 4-HB or related polymers in any organism.

The microbial production capabilities of 4-hydroxybutyrate were explored in two microbes, Escherichia coli and Saccharomyces cerevisiae, using in silico metabolic models of each organism. Potential pathways to 4-HB proceed via a succinate, succinyl-CoA, or alpha-ketoglutarate intermediate as shown in FIG. 1.

A first step in the 4-HB production pathway from succinate involves the conversion of succinate to succinic semialdehyde via an NADH- or NADPH-dependant succinic semialdehyde dehydrogenase. In E. coli, gabD is an NADP-dependant succinic semialdehyde dehydrogenase and is part of a gene cluster involved in 4-aminobutyrate uptake and degradation (Niegemann et al., Arch. Microbiol. 160:454460 (1993); Schneider et al., J. Bacteriol. 184:6976-6986(2002)). sad is believed to encode the enzyme for NAD-dependant succinic semialdehyde dehydrogenase activity (Marek and Henson, supra). S. cerevisiae contains only the NADPH-dependant succinic semialdehyde dehydrogenase, putatively assigned to UGA2, which localizes to the cytosol (Huh et al., Nature 425:686-691 (2003)). The maximum yield calculations assuming the succinate pathway to 4-HB in both E. coli and S. cerevisiae require only the assumption that a non-native 4-HB dehydrogenase has been added to their metabolic networks.

The pathway from succinyl-CoA to 4-hydroxybutyrate was described in U.S. Pat. No. 6,117,658 as part of a process for making polyhydroxyalkanoates comprising 4-hydroxybutyrate monomer units. Clostridium kluyveri is one example organism known to possess CoA-dependant succinic semialdehyde dehydrogenase activity (Sohling and Gottschalk, supra; Sohling and Gottschalk, supra). In this study, it is assumed that this enzyme, from C. kluyveri or another organism, is expressed in E. coli or S. cerevisiae along with a non-native or heterologous 4-HB dehydrogenase to complete the pathway from succinyl-CoA to 4-HB. The pathway from alpha-ketoglutarate to 4-HB was demonstrated in E. coli resulting in the accumulation of poly(4-hydroxybutyric acid) to 30% of dry cell weight (Song et al., supra). As E. coli and S. cerevisiae natively or endogenously possess both glutamate:succinic semialdehyde transaminase and glutamate decarboxylase (Coleman et al., J. Biol. Chem. 276:244-250 (2001)), the pathway from AKG to 4-HB can be completed in both organisms by assuming only that a non-native 4-HB dehydrogenase is present.

### Example II

### Biosynthesis of 1,4-Butanediol from Succinate and Alpha-ketoglutarate

This example illustrates the construction and biosynthetic production of 4-HB and BDO from microbial organisms. Pathways for 4-HB and BDO are disclosed herein.

There are several alternative enzymes that can be utilized in the pathway described above. The native or endogenous enzyme for conversion of succinate to succinyl-CoA (Step 1 in FIG. 1) can be replaced by a CoA transferase such as that encoded by the cat1 gene C. kluyveri (Sohling and Gottschalk, Eur. J Biochem. 212:121-127 (1993)), which functions in a similar manner to Step 9. However, the production of acetate by this enzyme may not be optimal, as it might be secreted rather than being converted back to acetyl-CoA. In this respect, it also can be beneficial to eliminate acetate formation in Step 9. As one alternative to this CoA transferase, a mechanism can be employed in which the 4-HB is first phosphorylated by ATP and then converted to the CoA derivative, similar to the acetate kinase/phosphotransacetylase pathway in E. coli for the conversion of acetate to acetyl-CoA. The net cost of this route is one ATP, which is the same as is required to regenerate acetyl-CoA from acetate. The enzymes phosphotransbutyrylase (ptb) and butyrate kinase (bk) are known to cany out these steps on the non-hydroxylated molecules for butyrate production in C. acetobutylicum (Cary et al., Appl Environ Microbiol 56:1576-1583 (1990); Valentine, R. C. and R. S. Wolfe, J Biol Chem. 235:1948-1952 (1960)). These enzymes are reversible, allowing synthesis to proceed in the direction of 4-HB.

BDO also can be produced via α-ketoglutarate in addition to or instead of through succinate. A described previously, and exemplified further below, one pathway to accomplish product biosynthesis is with the production of succinic semialdehyde via α-ketoglutarate using the endogenous enzymes (FIG. 1, Steps 4-5). An alternative is to use an α-ketoglutarate decarboxylase that can perform this conversion in one step (FIG. 1, Step 8; Tian et al., Proc Natl Acad Sci USA 102:10670-10675 (2005)).

For the construction of different strains of BDO-producing microbial organisms, a list of applicable genes was assembled for corroboration. Briefly, one or more genes within the 4-HB and/or BDO biosynthetic pathways were identified for each step of the complete BDO-producing pathway shown in FIG. 1, using available literature resources, the NCBI genetic database, and homology searches. The genes cloned and assessed in this study are presented below in in Table 6, along with the appropriate references and URL citations to the polypeptide sequence. As discussed further below, some genes were synthesized for codon optimization while others were cloned via PCR from the genomic DNA of the native or wild-type organism. For some genes both approaches were used, and in this case the native genes are indicated by an “n” suffix to the gene identification number when used in an experiment. Note that only the DNA sequences differ; the proteins are identical.

TABLE 6
Genes expressed in host BDO-producting microbial organisms.

| | Reaction | | | | | |
|---|---|---|---|---|---|---|
| Gene ID | number | Gene | | | Link to protein sequence | |
| number | (FIG. 1) | name | Source organism | Enzyme name | (path following www) | Reference |
| 0001 | 9 | Cat2 | Clostridium kluyveri | 4-hydroxybutyrate | ncbi.nlm.nih.gov/entrez/ | 1 |
| | | | DSM 555 | coenzyme A | viewer.fcgi?db=nuccore&id= | |
| | | | | transferase | 1228100 | |
| 0002 | 12/13 | adhE | Clostridium acetobutylicum | Aldehyde/alcohol | ncbi.nlm.nih.gov/entrez/ | 2 |
| | | | ATCC 824 | dehydrogenase | viewer.fcgi?db=protein&val= | |
| | | | | | 15004739 | |
| 0003 | 12/13 | adhE2 | Clostridium acetobutylicum | Aldehyde/alcohol | ncbi.nlm.nih.gov/entrez/ | 2 |
| | | | ATCC 824 | dehydrogenase | viewer.fcgi?val=NP_149325.1 | |
| 0004 | 1 | Cat1 | Clostridium kluyveri | Succinate | ncbi.nlm.nih.gov/entrez/ | 1 |
| | | | DSM 555 | coenzyme A | viewer.fcgi?db=nuccore&id= | |
| | | | | transferase | 1228100 | |
| 0008 | 6 | sucD | Clostridium kluyveri | Succinic | ncbi.nlm.nih.gov/entrez/ | 1 |
| | | | DSM 555 | semialdehyde | viewer.fcgi?db=nuccore&id= | |
| | | | | dehydrogenase | 1228100 | |
| | | | | (CoA-dependent) | | |
| 0009 | 7 | 4-HBd | Ralstonia eutropha | 4-hydroxybutyrate | ncbi.nlm.nih.gov/entrez/ | 2 |
| | | | H16 | dehydrogenase | viewer.fcgi?val=YP_726053.1 | |
| | | | | (NAD-dependent) | | |
| 0010 | 7 | 4-HBd | Clostridium kluyveri | 4-hydroxybutyrate | ncbi.nlm.nih.gov/entrez/ | 1 |
| | | | DSM 555 | dehydrogenase | viewer.fcgi?db=nuccore&id= | |
| | | | | (NAD-dependent) | 1228100 | |
| 0011 | 12/13 | adhE | E. coli | Aldehyde/alcohol | shigen.nig.ac.jp/ecoli/pec/ | |
| | | | | dehydrogenase | genes.List.DetailAction.do? | |
| | | | | | fromListFlag=true&featureType= | |
| | | | | | 1&orfId=1219 | |
| 0012 | 12/13 | yqhD | E. coli | Aldehyde/alcohol | shigen.nig.ac.jp/ecoli/pec/ | |
| | | | | dehydrogenase | genes.List.DetailAction.do | |
| 0013 | 13 | bdhB | Clostridium acetobutylicum | Butanol | ncbi.nlm.nih.gov/entrez/ | 2 |
| | | | ATCC 824 | dehydrogenase II | viewer.fcgi?val=NP_349891.1 | |
| 0020 | 11 | ptb | Clostridium acetobutylicum | Phospho- | ncbi.nlm.nih.gov/entrez/ | 2 |
| | | | ATCC 824 | transbutyrylase | viewer.fcgi?db=protein&id= | |
| | | | | | 15896327 | |
| 0021 | 10 | buk1 | Clostridium acetobutylicum | Butyrate kinase I | ncbi.nlm.nih.gov/entrez/ | 2 |
| | | | ATCC 824 | | viewer.fcgi?db=protein&id= | |
| | | | | | 20137334 | |
| 0022 | 10 | buk2 | Clostridium acetobutylicum | Butyrate kinase II | ncbi.nlm.nih.gov/entrez/ | 2 |
| | | | ATCC 824 | | viewer.fcgi?db=protein&id= | |
| | | | | | 20137415 | |
| 0023 | 13 | adhEm | isolated from metalibrary | Alcohol | | (37)d} |
| | | | of anaerobic sewage digester | dehydrogenase | | |
| | | | microbial consortia | | | |
| 0024 | 13 | adhE | Clostridium thermocellum | Alcohol | genome.jp/dbget- | |
| | | | | dehydrogenase | bin/www_bget?cth:Cthe_0423 | |
| 0025 | 13 | ald | Clostridium beijerinckii | Coenzyme A- | ncbi.nlm.nih.gov/entrez/ | (31)d} |
| | | | | acylating aldehyde | viewer.fcgi?db=protein&id= | |
| | | | | dehydrogenase | 49036681 | |
| 0026 | 13 | bdhA | Clostridium acetobutylicum | Butanol | ncbi.nlm.nih.gov/entrez/ | 2 |
| | | | ATCC 824 | dehydrogenase | viewer.fcgi?val=NP_349892.1 | |
| 0027 | 12 | bld | Clostridium | Butyraldehyde | ncbi.nlm.nih.gov/entrez/ | 4 |
| | | | saccharoperbutylacetonicum | dehydrogenase | viewer.fcgi?db=protein&id= | |
| | | | | | 31075383 | |
| 0028 | 13 | bdh | Clostridium | Butanol | ncbi.nlm.nih.gov/entrez/ | 4 |
| | | | saccharoperbutylacetonicum | dehydrogenase | viewer.fcgi?db=protein&id= | |
| | | | | | 124221917 | |
| 0029 | 12/13 | adhE | Clostridium tetani | Aldehyde/alcohol | genome.jp/dbget- | |
| | | | | dehydrogenase | bin/www_bget?ctc:CTC01366 | |
| 0030 | 12/13 | adhE | Clostridium perfringens | Aldehyde/alcohol | genome.jp/dbget- | |
| | | | | dehydrogenase | bin/www_bget?cpe:CPE2531 | |
| 0031 | 12/13 | adhE | Clostridium difficile | Aldehyde/alcohol | genome.jp/dbget- | |
| | | | | dehydrogenase | bin/www_bget?cdf:CD2966 | |
| 0032 | 8 | sucA | Mycobacterium bovis | α-ketoglutarate | ncbi.nlm.nih.gov/entrez/ | 5 |
| | | | BCG, Pasteur | decarboxylase | viewer.fcgi?val=YP_977400.1 | |
| 0033 | 9 | cat2 | Clostridium aminobutyricum | 4-hydroxybutyrate | ncbi.nlm.nih.gov/entrez/ | |
| | | | | coenzyme A | viewer.fcgi?db=protein&val= | |
| | | | | transferase | 6249316 | |
| 0034 | 9 | cat2 | Porphyromonas gingivalis | 4-hydroxybutyrate | ncbi.nlm.nih.gov/entrez/ | |
| | | | W83 | coenzyme A | viewer.fcgi?db=protein&val= | |
| | | | | transferase | 34541558 | |
| 0035 | 6 | sucD | Porphyromonas gingivalis | Succinic | ncbi.nlm.nih.gov/entrez/ | |
| | | | W83 | semialdehyde | viewer.fcgi?val=NP_904963.1 | |
| | | | | dehydrogenase | | |
| | | | | (CoA-dependent) | | |
| 0036 | 7 | 4-HBd | Porphyromonas gingivalis | NAD-dependent | ncbi.nlm.nih.gov/entrez/ | |
| | | | W83 | 4-hydroxybutyrate | viewer.fcgi?val=NP_904964.1 | |
| | | | | dehydrogenase | | |
| 0037 | 7 | gbd | Uncultured bacterium | 4-hydroxybutyrate | ncbi.nlm.nih.gov/entrez/ | 6 |
| | | | | dehydrogenase | viewer.fcgi?db=nuccore&id= | |
| | | | | | 5916168 | |
| 0038 | 1 | sucCD | E. coli | Succinyl-CoA | shigen.nig.ac.jp/ecoli/pec/ | |
| | | | | synthetase | genes.List.DetailAction.do | |

1 Sohling and Gottschalk, Eur. J. Biochem. 212: 121-127 (1993); Sohling and Gottschalk, J. Bacteriol. 178: 871-880 (1996)
2 Nolling et al., J., J. Bacteriol. 183: 4823-4838 (2001)
3 Pohlmann et al., Nat. Biotechnol. 24: 1257-1262 (2006)
4 Kosaka et al., Biosci. Biotechnol. Biochem. 71: 58-68 (2007)
5 Brosch et al., Proc. Natl. Acad. Sci. U.S.A. 104: 5596-5601 (2007)
6 Henne et al., Appl. Environ. Microbiol. 65: 3901-3907 (1999)

Expression Vector Construction for BDO pathway. Vector backbones and some strains were obtained from Dr. Rolf Lutz of Expressys (expressVs.de/). The vectors and strains are based on the pZ Expression System developed by Dr. Rolf Lutz and Prof. Hermann Bujard (Lutz, R. and H. Bujard, Nucleic Acids Res 25:1203-1210 (1997)). Vectors obtained were pZE13luc, pZA33luc, pZS*13luc and pZE22luc and contained the luciferase gene as a stuffer fragment. To replace the luciferase stuffer fragment with a lacZ-alpha fragment flanked by appropriate restriction enzyme sites, the luciferase stuffer fragment was first removed from each vector by digestion with EcoRI and XbaI. The lacZ-alpha fragment was PCR amplified from pUC19 with the following primers:

lacZalpha-RI
(SEQ ID NO: 1)
5′GACGAATTCGCTAGCAAGAGGAGAAGTCGACATGTCCAATTCACTGG
CCGTCGTTTTAC3′
lacZalpha 3′BB
(SEQ ID NO: 2)
5′-GACCCTAGGAAGCTTTCTAGAGTCGACCTATGCGGCATCAGAGCA
GA-3′.

This generated a fragment with a 5′ end of EcoRI site, NheI site, a Ribosomal Binding Site, a SalI site and the start codon. On the 3′ end of the fragment contained the stop codon, XbaI, HindIII, and AvrII sites. The PCR product was digested with EcoRI and AvrII and ligated into the base vectors digested with EcoRI and XbaI (XbaI and AvrII have compatible ends and generate a non-site). Because NheI and XbaI restriction enzyme sites generate compatible ends that can be ligated together (but generate a NheI/XbaI non-site that is not digested by either enzyme), the genes cloned into the vectors could be “Biobricked” together (openwetware.org/wiki/Synthetic_Biology:BioBricks). Briefly, this method allows joining an unlimited number of genes into the vector using the same 2 restriction sites (as long as the sites do not appear internal to the genes), because the sites between the genes are destroyed after each addition.

All vectors have the pZ designation followed by letters and numbers indication the origin of replication, antibiotic resistance marker and promoter/regulatory unit. The origin of replication is the second letter and is denoted by E for ColE1, A for p15A and S for pSC101-based origins. The first number represents the antibiotic resistance marker (1 for Ampicillin, 2 for Kanamycin, 3 for Chloramphenicol, 4 for Spectinomycin and 5 for Tetracycline). The final number defines the promoter that regulated the gene of interest (1 for P_{LtetO-1}, 2 for P_{LlacO-1}, 3 for P_{A1lacO-1}, and 4 for P_{lac/ara-1}). The MCS and the gene of interest follows immediately after. For the work discussed here we employed two base vectors, pZA33 and pZE13, modified for the biobricks insertions as discussed above. Once the gene(s) of interest have been cloned into them, resulting plasmids are indicated using the four digit gene codes given in Table 6; e.g., pZA33-XXXX-YYYY-.

Host Strain Construction. The parent strain in all studies described here is E. coli K-12 strain MG1655. Markerless deletion strains in adhE, gabD, and aldA were constructed under service contract by a third party using the redET method (Datsenko, K. A. and B. L. Wanner, Proc Natl Acad Sci USA 97:6640-6645 (2000)). Subsequent strains were constructed via bacteriophage P1 mediated transduction (Miller, J. Experiments in Molecular Genetics, Cold Spring Harbor Laboratories, New York (1973)). Strain C600Z1 (laci^{q}, PN25-tetR, Sp^{R}, lacY1, leuB6, mcrB+, supE44, thi-1, thr-1, tonA21) was obtained from Expressys and was used as a source of a lacI^{q} allele for P1 transduction. Bacteriophage P1vir was grown on the C600Z1 E. coli strain, which has the spectinomycin resistance gene linked to the lacI^{q}. The P1 lysate grown on C600Z1 was used to infect MG1655 with selection for spectinomycin resistance. The spectinomycin resistant colonies were then screened for the linked lacI^{q} by determining the ability of the transductants to repress expression of a gene linked to a P_{A1lacO-1} promoter. The resulting strain was designated MG1655 lacI^{q}. A similar procedure was used to introduce lacI^{Q} into the deletion strains.

Production of 4-HB From Succinate. For construction of a 4-HB producer from succinate, genes encoding steps from succinate to 4-HB and 4-HB-CoA (1, 6, 7, and 9 in FIG. 1) were assembled onto the pZA33 and pZE13 vectors as described below. Various combinations of genes were assessed, as well as constructs bearing incomplete pathways as controls (Tables 7 and 8). The plasmids were then transformed into host strains containing lacI^{Q}, which allow inducible expression by addition of isopropyl β-D-1-thiogalactopyranoside (IPTG). Both wild-type and hosts with deletions in genes encoding the native succinic semialdehyde dehydrogenase (step 2 in FIG. 1) were tested.

Activity of the heterologous enzymes were first tested in in vitro assays, using strain MG1655 lacI^{Q} as the host for the plasmid constructs containing the pathway genes. Cells were grown aerobically in LB media (Difco) containing the appropriate antibiotics for each construct, and induced by addition of IPTG at 1 mM when the optical density (OD600) reached approximately 0.5. Cells were harvested after 6 hours, and enzyme assays conducted as discussed below.

In Vitro Enzyme Assays. To obtain crude extracts for activity assays, cells were harvested by centrifugation at 4,500 rpm (Beckman-Coulter, Allegera X-15R) for 10 min. The pellets were resuspended in 0.3 mL BugBuster (Novagen) reagent with benzonase and lysozyme, and lysis proceeded for 15 minutes at room temperature with gentle shaking. Cell-free lysate was obtained by centrifugation at 14,000 rpm (Eppendorf centrifuge 5402) for 30 min at 4° C. Cell protein in the sample was determined using the method of Bradford et al., Anal. Biochem. 72:248-254 (1976), and specific enzyme assays conducted as described below. Activities are reported in Units/mg protein, where a unit of activity is defined as the amount of enzyme required to convert 1 μmol of substrate in 1 min. at room temperature. In general, reported values are averages of at least 3 replicate assays.

Succinyl-CoA transferase (Cat1) activity was determined by monitoring the formation of acetyl-CoA from succinyl-CoA and acetate, following a previously described procedure Sohling and Gottschalk, J. Bacteriol. 178:871-880 (1996). Succinyl-CoA synthetase (SucCD) activity was determined by following the formation of succinyl-CoA from succinate and CoA in the presence of ATP The experiment followed a procedure described by Cha and Parks, J. Biol. Chem. 239:1961-1967 (1964). CoA-dependent succinate semialdehyde dehydrogenase (SucD) activity was determined by following the conversion of NAD to NADH at 340 nm in the presence of succinate semialdehyde and CoA (Sohling and Gottschalk, Eur. J. Biochem. 212:121-127 (1993)). 4-HB dehydrogenase (4-HBd) enzyme activity was determined by monitoring the oxidation of NADH to NAD at 340 nm in the presence of succinate semialdehyde. The experiment followed a published procedure Gerhardt et al. Arch. Microbiol. 174:189-199 (2000). 4-HB CoA transferase (Cat2) activity was determined using a modified procedure from Scherf and Buckel, Appl. Environ. Microbiol. 57:2699-2702(1991). The formation of 4-HB-CoA or butyryl-CoA formation from acetyl-CoA and 4-HB or butyrate was determined using HPLC.

Alcohol (ADH) and aldehyde (ALD) dehydrogenase was assayed in the reductive direction using a procedure adapted from several literature sources (Durre et al., FEMS Microbiol. Rev. 17:251-262(1995); Palosaari and Rogers, J. Bacteriol. 170:2971-2976 (1988) and Welch et al., Arch. Biochem. Biophys. 273:309-318 (1989). The oxidation of NADH is followed by reading absorbance at 340 nM every four seconds for a total of 240 seconds at room temperature. The reductive assays were performed in 100 mM MOPS (adjusted to pH 7.5 with KOH), 0.4 mM NADH, and from 1 to 50 μl of cell extract. The reaction is started by adding the following reagents: 100 μl of 100 mM acetaldehyde or butyraldehyde for ADH, or 100 μl of 1 mM acetyl-CoA or butyryl-CoA for ALD. The Spectrophotometer is quickly blanked and then the kinetic read is started. The resulting slope of the reduction in absorbance at 340 nM per minute, along with the molar extinction coefficient of NAD(P)H at 340 nM (6000) and the protein concentration of the extract, can be used to determine the specific activity.

The enzyme activity of PTB is measured in the direction of butyryl-CoA to butyryl-phosphate as described in Cary et al. J. Bacteriol. 170:4613-4618 (1988). It provides inorganic phosphate for the conversion, and follows the increase in free CoA with the reagent 5,5′-dithiobis-(2-nitrobenzoic acid), or DTNB. DTNB rapidly reacts with thiol groups such as free CoA to release the yellow-colored 2-nitro-5-mercaptobenzoic acid (TNB), which absorbs at 412 nm with a molar extinction coefficient of 14,140 M cm⁻¹. The assay buffer contained 150 mM potassium phosphate at pH 7.4, 0.1 mM DTNB, and 0.2 mM butyryl-CoA, and the reaction was started by addition of 2 to 50 μL cell extract. The enzyme activity of BK is measured in the direction of butyrate to butyryl-phosphate formation at the expense of ATP The procedure is similar to the assay for acetate kinase previously described Rose et al., J. Biol. Chem. 211:737-756 (1954). However it has been found that another acetate kinase enzyme assay protocol provided by Sigma to be more useful and sensitive. This assay links conversion of ATP to ADP by acetate kinase to the linked conversion of ADP and phosphoenolpyruvate (PEP) to ATP and pyruvate by pyruvate kinase, followed by the conversion of pyruvate and NADH to lactate and NAD+ by lactate dehydrogenase. Substituting butyrate for acetate is the only major modification to allow the assay to follow BK enzyme activity. The assay mixture contained 80 mM triethanolamine buffer at pH 7.6, 200 mM sodium butyrate, 10 mM MgCl2, 0.1 mM NADH, 6.6 mM ATP, 1.8 mM phosphoenolpyruvate. Pyruvate kinase, lactate dehydrogenase, and myokinase were added according to the manufacturer's instructions. The reaction was started by adding 2 to 50 μL cell extract, and the reaction was monitored based on the decrease in absorbance at 340 nm indicating NADH oxidation.

Analysis of CoA Derivatives by HPLC. An HPLC based assay was developed to monitor enzymatic reactions involving coenzyme A (CoA) transfer. The developed method allowed enzyme activity characterization by quantitative determination of CoA, acetyl CoA (AcCoA), butyryl CoA (BuCoA) and 4-hydroxybutyrate CoA (4-HBCoA) present in in-vitro reaction mixtures. Sensitivity down to low μM was achieved, as well as excellent resolution of all the CoA derivatives of interest.

Chemical and sample preparation was performed as follows. Briefly, CoA, AcCoA, BuCoA and all other chemicals, were obtained from Sigma-Aldrich. The solvents, methanol and acetonitrile, were of HPLC grade. Standard calibration curves exhibited excellent linearity in the 0.01-1 mg/nL concentration range. Enzymatic reaction mixtures contained 100 mM Tris HCl buffer (pH 7), aliquots were taken at different time points, quenched with formic acid (0.04% final concentration) and directly analyzed by HPLC.

HPLC analysis was performed using an Agilent 1100 HPLC system equipped with a binary pump, degasser, thermostated autosampler and column compartment, and diode array detector (DAD), was used for the analysis. A reversed phase column, Kromasil 100 5 um C18, 4.6×150 mm (Peeke Scientific), was employed. 25 mM potassium phosphate (pH 7) and methanol or acetonitrile, were used as aqueous and organic solvents at 1 mL/min flow rate. Two methods were developed: a short one with a faster gradient for the analysis of well-resolved CoA, AcCoA and BuCoA, and a longer method for distinguishing between closely eluting AcCoA and 4-HBCoA. Short method employed acetonitrile gradient (0 min-5%, 6 min-30%, 6.5 min-5%, 10 min-5%) and resulted in the retention times 2.7, 4.1 and 5.5 min for CoA, AcCoA and BuCoA, respectively. In the long method methanol was used with the following linear gradient: 0 min-5%, 20 min-35%, 20.5 min-5%, 25 min-5%. The retention times for CoA, AcCoA, 4-HBCoA and BuCoA were 5.8, 8.4, 9.2 and 16.0 min, respectively. The injection volume was 5 μL, column temperature 30° C., and UV absorbance was monitored at 260 nm.

The results demonstrated activity of each of the four pathway steps (Table 7), though activity is clearly dependent on the gene source, position of the gene in the vector, and the context of other genes with which it is expressed. For example, gene 0035 encodes a succinic semialdehyde dehydrogenase that is more active than that encoded by 0008, and 0036 and 0010n are more active 4-HB dehydrogenase genes than 0009. There also seems to be better 4-HB dehydrogenase activity when there is another gene preceding it on the same operon.

TABLE 7
In vitro enzyme activities in cell extracts from MG1655 lacI^{Q} containing the plasmids expressing genes
in the 4-HB-CoA pathway. Activities are reported in Units/mg protein, where a unit of activity is defined
as the amount of enzyme required to convert 1 μmol of substrate in 1 min. at room temperature.

| Sample # | pZE13 (a) | pZA33 (b) | OD600 | Cell Prot (c) | Cat1 | SucD | 4HBd | Cat2 |
|---|---|---|---|---|---|---|---|---|
| 1 | cat1 (0004) | | 2.71 | 6.43 | 1.232 | 0.00 | | |
| 2 | cat1 (0004)-sucD (0035) | | 2.03 | 5.00 | 0.761 | 2.57 | | |
| 3 | cat1 (0004)-sucD (0008) | | 1.04 | 3.01 | 0.783 | 0.01 | | |
| 4 | sucD (0035) | | 2.31 | 6.94 | | 2.32 | | |
| 5 | sucD (0008) | | 1.10 | 4.16 | | 0.05 | | |
| 6 | | 4hbd (0009) | 2.81 | 7.94 | 0.003 | | 0.25 | |
| 7 | | 4hbd (0036) | 2.63 | 7.84 | | | 3.31 | |
| 8 | | 4hbd (0010n) | 2.00 | 5.08 | | | 2.57 | |
| 9 | cat1 (0004)-sucD (0035) | 4hbd (0009) | 2.07 | 5.04 | 0.600 | 1.85 | 0.01 | |
| 10 | cat1 (0004)-sucD (0035) | 4hbd (0036) | 2.08 | 5.40 | 0.694 | 1.73 | 0.41 | |
| 11 | cat1 (0004)-sucD (0035) | 4hbd (0010n) | 2.44 | 4.73 | 0.679 | 2.28 | 0.37 | |
| 12 | cat1 (0004)-sucD (0008) | 4hbd (0009) | 1.08 | 3.99 | 0.572 | −0.01 | 0.02 | |
| 13 | cat1 (0004)-sucD (0008) | 4hbd (0036) | 0.77 | 2.60 | 0.898 | −0.01 | 0.04 | |
| 14 | cat1 (0004)-sucD (0008) | 4hbd (0010n) | 0.63 | 2.47 | 0.776 | 0.00 | 0.00 | |
| 15 | | cat2 (0034) | 2.56 | 7.86 | | | | 1.283 |
| 16 | | cat2(0034)-4hbd(0036) | 3.13 | 8.04 | | | 24.86 | 0.993 |
| 17 | | cat2(0034)-4hbd(0010n) | 2.38 | 7.03 | | | 7.45 | 0.675 |
| 18 | | 4hbd(0036)-cat2(0034) | 2.69 | 8.26 | | | 2.15 | 7.490 |
| 19 | | 4hbd(0010n)-cat2(0034) | 2.44 | 6.59 | | | 0.59 | 4.101 |

Genes expressed from Plac on pZE13, a high-copy plasmid with colE1 origin and ampicillin resistance.
Gene identification numbers are as given in Table 6
Genes expressed from Plac on pZA33, a medium-copy plasmid with pACYC origin and chloramphenicol resistance.
(c) Cell protein given as mg protein per mL extract.

Recombinant strains containing genes in the 4-HB pathway were then evaluated for the ability to produce 4-HB in vivo from central metabolic intermediates. Cells were grown anaerobically in LB medium to OD600 of approximately 0.4, then induced with 1 mM IPTG. One hour later, sodium succinate was added to 10 mM and samples taken for analysis following an additional 24 and 48 hours. 4-HB in the culture broth was analyzed by GC-MS as described below. The results indicate that the recombinant strain can produce over 2 mM 4-HB after 24 hours, compared to essentially zero in the control strain (Table 8).

TABLE 8
Production of 4-HB from succinate in E. coli strains harboring plasmids expressing various combinations of 4-HB pathway genes.

| | | | | 24 Hours | | | 48 Hours | | |
|---|---|---|---|---|---|---|---|---|---|
| | | | | | 4HB, | 4HB | | 4HB, | 4HB |
| Sample # | Host Strain | pZE13 | pZA33 | OD600 | μM | norm. (a) | OD600 | μM | norm. (a) |
| 1 | MG1655 lacIq | cat1 (0004)-sucD (0035) | 4hbd (0009) | 0.47 | 487 | 1036 | 1.04 | 1780 | 1711 |
| 2 | MG1655 lacIq | cat1 (0004)-sucD (0035) | 4hbd (0027) | 0.41 | 111 | 270 | 0.99 | 214 | 217 |
| 3 | MG1655 lacIq | cat1 (0004)-sucD (0035) | 4hbd (0036) | 0.47 | 863 | 1835 | 0.48 | 2152 | 4484 |
| 4 | MG1655 lacIq | cat1 (0004)-sucD (0035) | 4hbd (0010n) | 0.46 | 956 | 2078 | 0.49 | 2221 | 4533 |
| 5 | MG1655 lacIq | cat1 (0004)-sucD (0008) | 4hbd (0009) | 0.38 | 493 | 1296 | 0.37 | 1338 | 3616 |
| 6 | MG1655 lacIq | cat1 (0004)-sucD (0008) | 4hbd (0027) | 0.32 | 26 | 81 | 0.27 | 87 | 323 |
| 7 | MG1655 lacIq | cat1 (0004)-sucD (0008) | 4hbd (0036) | 0.24 | 506 | 2108 | 0.31 | 1448 | 4672 |
| 8 | MG1655 lacIq | cat1 (0004)-sucD (0008) | 4hbd (0010n) | 0.24 | 78 | 324 | 0.56 | 233 | 416 |
| 9 | MG1655 lacIq gabD | cat1 (0004)-sucD (0035) | 4hbd (0009) | 0.53 | 656 | 1237 | 1.03 | 1643 | 1595 |
| 10 | MG1655 lacIq gabD | cat1 (0004)-sucD (0035) | 4hbd (0027) | 0.44 | 92 | 209 | 0.98 | 214 | 218 |
| 11 | MG1655 lacIq gabD | cat1 (0004)-sucD (0035) | 4hbd (0036) | 0.51 | 1072 | 2102 | 0.97 | 2358 | 2431 |
| 12 | MG1655 lacIq gabD | cat1 (0004)-sucD (0035) | 4hbd (0010n) | 0.51 | 981 | 1924 | 0.97 | 2121 | 2186 |
| 13 | MG1655 lacIq gabD | cat1 (0004)-sucD (0008) | 4hbd (0009) | 0.35 | 407 | 1162 | 0.77 | 1178 | 1530 |
| 14 | MG1655 lacIq gabD | cat1 (0004)-sucD (0008) | 4hbd (0027) | 0.51 | 19 | 36 | 1.07 | 50 | 47 |
| 15 | MG1655 lacIq gabD | cat1 (0004)-sucD (0008) | 4hbd (0036) | 0.35 | 584 | 1669 | 0.78 | 1350 | 1731 |
| 16 | MG1655 lacIq gabD | cat1 (0004)-sucD (0008) | 4hbd (0010n) | 0.32 | 74 | 232 | 0.82 | 232 | 283 |
| 17 | MG1655 lacIq | vector only | vector only | 0.8 | 1 | 2 | 1.44 | 3 | 2 |
| 18 | MG1655 lacIq gabD | vector only | vector only | 0.89 | 1 | 2 | 1.41 | 7 | 5 |

(a) Normalized 4-HB concentration, μM/OD600 units

An alternate to using a CoA transferase (cat1) to produce succinyl-CoA from succinate is to use the native E. coli sucCD genes, encoding succinyl-CoA synthetase. This gene cluster was cloned onto pZE13 along with candidate genes for the remaining steps to 4-HB to create pZE13-0038-0035-0036.

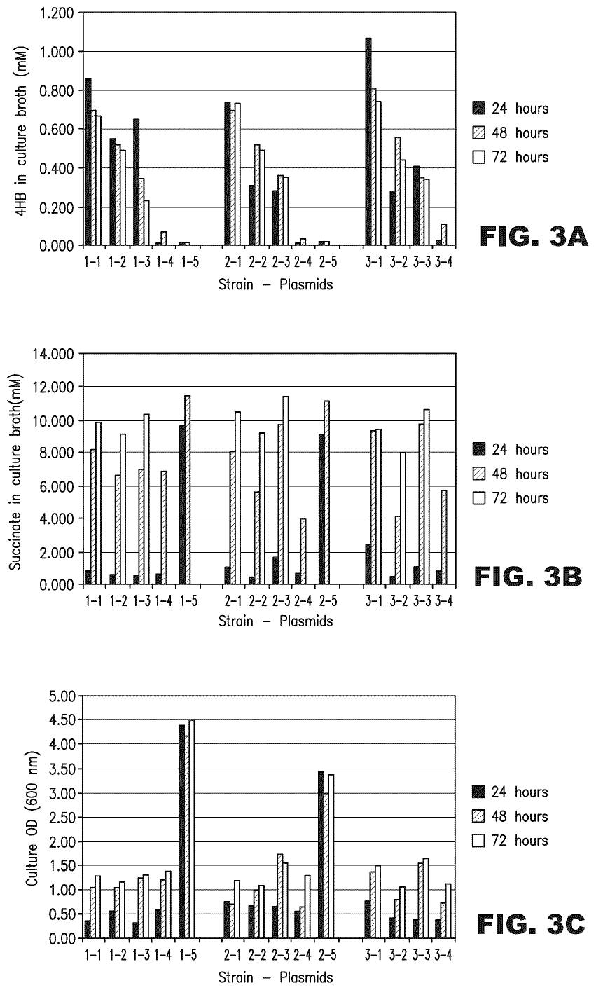

Production of 4-HB from Glucose. Although the above experiments demonstrate a functional pathway to 4-HB from a central metabolic intermediate (succinate), an industrial process would require the production of chemicals from low-cost carbohydrate feedstocks such as glucose or sucrose. Thus, the next set of experiments was aimed to determine whether endogenous succinate produced by the cells during growth on glucose could fuel the 4-HB pathway. Cells were grown anaerobically in M9 minimal medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂) supplemented with 20 g/L glucose, 100 mM 3-(N-morpholino)propanesulfonic acid (MOPS) to improve the buffering capacity, 10 μg/mL thiamine, and the appropriate antibiotics. 0.25 mM IPTG was added when OD600 reached approximately 0.2, and samples taken for 4-HB analysis every 24 hours following induction. In all cases 4-HB plateaued after 24 hours, with a maximum of about 1 mM in the best strains (FIG. 3a), while the succinate concentration continued to rise (FIG. 3b). This indicates that the supply of succinate to the pathway is likely not limiting, and that the bottleneck may be in the activity of the enzymes themselves or in NADH availability. 0035 and 0036 are clearly the best gene candidates for CoA-dependent succinic semialdehyde dehydrogenase and 4-HB dehydrogenase, respectively. The elimination of one or both of the genes encoding known (gabD) or putative (aldA)native succinic semialdehyde dehydrogenases had little effect on performance. Finally, it should be noted that the cells grew to a much lower OD in the 4-HB-producing strains than in the controls (FIG. 3c).

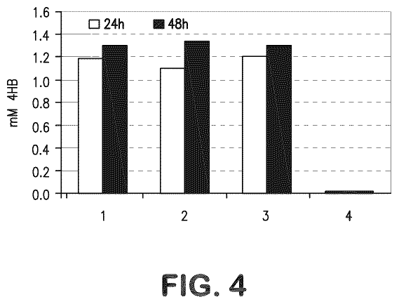

An alternate pathway for the production of 4-HB from glucose is via α-ketoglutarate. We explored the use of an α-ketoglutarate decarboxylase from Mycobacterium tuberculosis Tian et al., Proc. Natl. Acad. Sci. USA 102:10670-10675 (2005) to produce succinic semialdehyde directly from α-ketoglutarate (step 8 in FIG. 1). To demonstrate that this gene (0032) was functional in vivo, we expressed it on pZE13 in the same host as 4-HB dehydrogenase (gene 0036) on pZA33. This strain was capable of producing over 1.0 mM 4-HB within 24 hours following induction with 1 mM IPTG (FIG. 4). Since this strain does not express a CoA-dependent succinic semialdehyde dehydrogenase, the possibility of succinic semialdehyde production via succinyl-CoA is eliminated. It is also possible that the native genes responsible for producing succinic semialdehyde could function in this pathway (steps 4 and 5 in FIG. 1); however, the amount of 4-HB produced when the pZE13-0032 plasmid was left out of the host is the negligible.

Production of BDO from 4-HB. The production of BDO from 4-HB required two reduction steps, catalyzed by dehydrogenases. Alcohol and aldehyde dehydrogenases (ADH and ALD, respectively) are NAD+/H and/or NADP+/H-dependent enzymes that together can reduce a carboxylic acid group on a molecule to an alcohol group, or in reverse, can perform the oxidation of an alcohol to a carboxylic acid. This biotransformation has been demonstrated in wild-type Clostridium acetobutylicum (Jewell et al., Current Microbiology, 13:215-19 (1986)), but neither the enzymes responsible nor the genes responsible were identified. In addition, it is not known whether activation to 4-HB-CoA is first required (step 9 in FIG. 1), or if the aldehyde dehydrogenase (step 12) can act directly on 4-HB. We developed a list of candidate enzymes from C. acetobutylicum and related organisms based on known activity with the non-hydroxylated analogues to 4-HB and pathway intermediates, or by similarity to these characterized genes (Table 6). Since some of the candidates are multifunctional dehydrogenases, they could potentially catalyze both the NAD(P)H-dependent reduction of the acid (or CoA-derivative) to the aldehyde, and of the aldehyde to the alcohol. Before beginning work with these genes in E. coli, we first validated the result referenced above using C. acetobutylicum ATCC 824. Cells were grown in Schaedler broth (Accumedia, Lansing, MI) supplemented with 10 mM 4-HB, in an anaerobic atmosphere of 10% CO₂, 10% H₂, and 800% N₂ at 30° C. Periodic culture samples were taken, centrifuged, and the broth analyzed for BDO by GC-MS as described below. BDO concentrations of 0.1 mM, 0.9 mM, and 1.5 mM were detected after 1 day, 2 days, and 7 days incubation, respectively. No BDO was detected in culture grown without 4-HB addition. To demonstrate that the BDO produced was derived from glucose, we grew the best BDO producing strain MG1655 lacI^{Q} pZE13-0004-0035-0002 pZA33-0034-0036 in M9 minimal medium supplemented with 4 g/L uniformly labeled ¹³C-glucose. Cells were induced at OD of 0.67 with 1 mM IPTG, and a sample taken after 24 hours. Analysis of the culture supernatant was performed by mass spectrometry.

Gene candidates for the 4-HB to BDO conversion pathway were next tested for activity when expressed in the E. coli host MG1655 lac^{Q}. Recombinant strains containing each gene candidate expressed on pZA33 were grown in the presence of 0.25 mM IPTG for four hours at 37° C. to fully induce expression of the enzyme. Four hours after induction, cells were harvested and assayed for ADH and ALD activity as described above. Since 4-HB-CoA and 4-hydroxybutyraldehyde are not available commercially, assays were performed using the non-hydroxylated substrates (Table 9). The ratio in activity between 4-carbon and 2-carbon substrates for C. acetobutylicum adhE2 (0002) and E. coli adhE (0011) were similar to those previously reported in the literature a Atsumi et al., Biochim. Biophys. Acta. 1207:1-11(1994).

TABLE 9
In vitro enzyme activities in cell extracts from MG1655
lacI^{Q} containing pZA33 expressing gene candidates for
aldehyde and alcohol dehydrogenases. Activities are expressed in
μmol min⁻¹ mg cell protein⁻¹.

| | | Aldehyde | | | |
|---|---|---|---|---|---|
| | | dehydrogenase | | Alcohol | |
| | | Butyryl- | Acetyl- | dehydrogenase | |
| Gene | Substrate | CoA | CoA | Butyraldehyde | Acetaldehyde |

| | | | | |
|---|---|---|---|---|
| 0002 | 0.0076 | 0.0046 | 0.0264 | 0.0247 |
| 0003n | 0.0060 | 0.0072 | 0.0080 | 0.0075 |
| 0011 | 0.0069 | 0.0095 | 0.0265 | 0.0093 |
| 0013 | N.D. | N.D. | 0.0130 | 0.0142 |
| 0023 | 0.0089 | 0.0137 | 0.0178 | 0.0235 |
| 0025 | 0 | 0.0001 | N.D. | N.D. |
| 0026 | 0 | 0.0005 | 0.0024 | 0.0008 |

N.D., not determined.

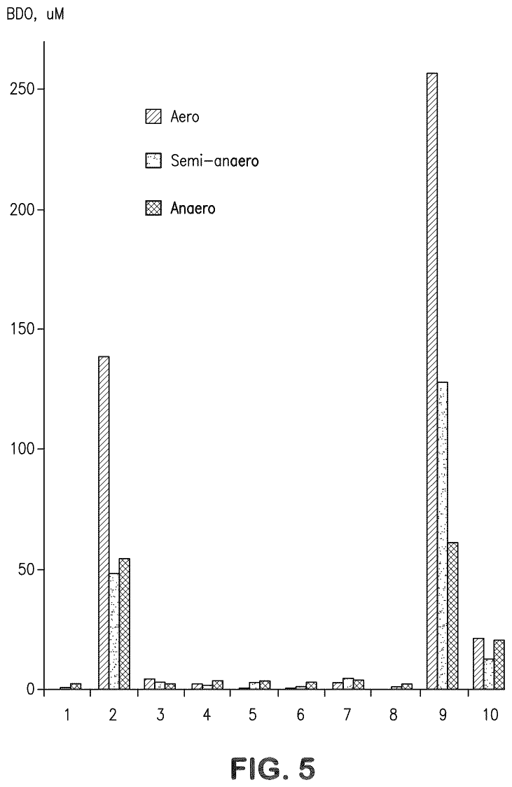

For the BDO production experiments, cat2 from Porphyromonas gingivalis W83 (gene 0034) was included on pZA33 for the conversion of 4-HB to 4-HB-CoA, while the candidate dehydrogenase genes were expressed on pZE13. The host strain was MG1655 lac. Along with the alcohol and aldehyde dehydrogenase candidates, we also tested the ability of CoA-dependent succinic semialdehyde dehydrogenases (sucD) to function in this step, due to the similarity of the substrates. Cells were grown to an OD of about 0.5 in LB medium supplemented with 10 mM 4-HB, induced with 1 mM IPTG, and culture broth samples taken after 24 hours and analyzed for BDO as described below. The best BDO production occurred using adhE2 from C. acetobutylicum, sucD from C. kluyveri, or sucD from P. gingivalis (FIG. 5). Interestingly, the absolute amount of BDO produced was higher under aerobic conditions; however, this is primarily due to the lower cell density achieved in anaerobic cultures. When normalized to cell OD, the BDO production per unit biomass is higher in anaerobic conditions (Table 10).

TABLE 10
Absolute and normalized BDO concentrations from cultures of cells
expressing adhE2 from C. acetobutylicum, sucD from C. kluyveri,
or sucD from P. gingivalis (data from experiments 2, 9, and 10
in FIG. 3), as well as the negative control (experiment 1).

| Gene | | BDO | OD | |
|---|---|---|---|---|
| expressed | Conditions | (μM) | (600 nm) | BDO/OD |
| none | Aerobic | 0 | 13.4 | 0 |
| none | Microaerobic | 0.5 | 6.7 | 0.09 |
| none | Anaerobic | 2.2 | 1.26 | 1.75 |
| 0002 | Aerobic | 138.3 | 9.12 | 15.2 |
| 0002 | Microaerobic | 48.2 | 5.52 | 8.73 |
| 0002 | Anaerobic | 54.7 | 1.35 | 40.5 |
| 0008n | Aerobic | 255.8 | 5.37 | 47.6 |
| 0008n | Microaerobic | 127.9 | 3.05 | 41.9 |
| 0008n | Anaerobic | 60.8 | 0.62 | 98.1 |
| 0035 | Aerobic | 21.3 | 14.0 | 1.52 |
| 0035 | Microaerobic | 13.1 | 4.14 | 3.16 |
| 0035 | Anaerobic | 21.3 | 1.06 | 20.1 |

As discussed above, it may be advantageous to use a route for converting 4-HB to 4-HB-CoA that does not generate acetate as a byproduct. To this aim, we tested the use of phosphotransbutyrylase (ptb) and butyrate kinase (bk) from C. acetobutylicum to cany out this conversion via steps 10 and 11 in FIG. 1. The native ptb/bk operon from C. acetobutylicum (genes 0020 and 0021) was cloned and expressed in pZA33. Extracts from cells containing the resulting construct were taken and assayed for the two enzyme activities as described herein. The specific activity of BK was approximately 65 U/mg, while the specific activity of PTB was approximately 5 U/mg. One unit (U) of activity is defined as conversion of 1 μM substrate in 1 minute at room temperature. Finally, the construct was tested for participation in the conversion of 4-HB to BDO. Host strains were transformed with the pZA33-0020-0021 construct described and pZE13-0002, and compared to use of cat2 in BDO production using the aerobic procedure used above in FIG. 5. The BK/PTB strain produced 1 mM BDO, compared to 2 mM when using cat2 (Table 11). Interestingly, the results were dependent on whether the host strain contained a deletion in the native adhE gene.

TABLE 11
Absolute and normalized BDO concentrations from cultures
of cells expressing adhE2 from C. acetobutylicum in pZE13
along with either cat2 from P. gingivalis (0034) or the PTB/
BK genes from C. acetobutylicum on pZA33. Host strains were
either MG1655 lacI^{Q} or MG1655 ΔadhE lacI^{Q}.

| | | BDO | OD | |
|---|---|---|---|---|
| Genes | Host Strain | (μM) | (600 nm) | BDO/OD |
| 0034 | MG1655 lacI^{Q} | 0.827 | 19.9 | 0.042 |
| 0020 + 0021 | MG1655 lacI^{Q} | 0.007 | 9.8 | 0.0007 |
| 0034 | MG1655 ΔadhE | 2.084 | 12.5 | 0.166 |
| | lacI^{Q} | | | |
| 0020 + 0021 | MG1655 ΔadhE | 0.975 | 18.8 | 0.052 |
| | lacI^{Q} | | | |

Production of BDO from Glucose. The final step of pathway corroboration is to express both the 4-HB and BDO segments of the pathway in E. coli and demonstrate production of BDO in glucose minimal medium. New plasmids were constructed so that all the required genes fit on two plasmids. In general, cat1, adhE, and sucD genes were expressed from pZE13, and cat2 and 4-HBd were expressed from pZA33. Various combinations of gene source and gene order were tested in the MG1655 lacI^{Q} background. Cells were grown anaerobically in M9 minimal medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂) supplemented with 20 g/L glucose, 100 mM 3-(N-morpholino)propanesulfonic acid (MOPS) to improve the buffering capacity, 10 μg/mL thiamine, and the appropriate antibiotics. 0.25 mM IPTG was added approximately 15 hours following inoculation, and culture supernatant samples taken for BDO, 4-HB, and succinate analysis 24 and 48 hours following induction. The production of BDO appeared to show a dependency on gene order (Table 12). The highest BDO production, over 0.5 mM, was obtained with cat2 expressed first, followed by 4-Bd on pZA33, and cat1 followed by P. gingivalis sucD on pZE13. The addition of C. acetobutylicum adhE2 in the last position on pZE13 resulted in slight improvement. 4-HB and succinate were also produced at higher concentrations.

TABLE 12
Production of BDO, 4-HB, and succinate in recombinant E. coli strains expressing combinations of BDO
pathway genes, grown in minimal medium supplemented with 20 g/L glucose. Concentrations are given in mM.

| | | | Induc- | 24 Hours | | | | 48 Hours | | | |
|---|---|---|---|---|---|---|---|---|---|---|---|
| Sam- | | | tion | OD600 | | | | OD600 | | | |
| ple | pZE13 | pZA33 | OD | nm | Su | 4HB | BDO | nm | Su | 4HB | BDO |
| 1 | cat1(0004)-sucD(0035) | 4hbd (0036)-cat2(0034) | 0.92 | 1.29 | 5.44 | 1.37 | 0.240 | 1.24 | 6.42 | 1.49 | 0.280 |
| 2 | cat1(0004)-sucD(0008N) | 4hbd (0036)-cat2(0034) | 0.36 | 1.11 | 6.90 | 1.24 | 0.011 | 1.06 | 7.63 | 1.33 | 0.011 |
| 3 | adhE(0002)-cat1(0004)-sucD(0035) | 4hbd (0036)-cat2(0034) | 0.20 | 0.44 | 0.34 | 1.84 | 0.050 | 0.60 | 1.93 | 2.67 | 0.119 |
| 4 | cat1(0004)-sucD(0035)-adhE(0002) | 4hbd (0036)-cat2(0034) | 1.31 | 1.90 | 9.02 | 0.73 | 0.073 | 1.95 | 9.73 | 0.82 | 0.077 |
| 5 | adhE(0002)-cat1(0004)-sucD(0008N) | 4hbd (0036)-cat2(0034) | 0.17 | 0.45 | 1.04 | 1.04 | 0.008 | 0.94 | 7.13 | 1.02 | 0.017 |
| 6 | cat1(0004)-sucD(0008N)-adhE(0002) | 4hbd (0036)-cat2(0034) | 1.30 | 1.77 | 10.47 | 0.25 | 0.004 | 1.80 | 11.49 | 0.28 | 0.003 |
| 7 | cat1(0004)-sucD(0035) | cat2(0034)-4hbd(0036) | 1.09 | 1.29 | 5.63 | 2.15 | 0.461 | 1.38 | 6.66 | 2.30 | 0.520 |
| 8 | cat1(0004)-sucD(0008N) | cat2(0034)-4hbd(0036) | 1.81 | 2.01 | 11.28 | 0.02 | 0.000 | 2.24 | 11.13 | 0.02 | 0.000 |
| 9 | adhE(0002)-cat1(0004)-sucD(0035) | cat2(0034)-4hbd(0036) | 0.24 | 1.99 | 2.02 | 2.32 | 0.106 | 0.89 | 4.85 | 2.41 | 0.186 |
| 10 | cat1(0004)-sucD(0035)-adhE(0002) | cat2(0034)-4hbd(0036) | 0.98 | 1.17 | 5.30 | 2.08 | 0.569 | 1.33 | 6.15 | 2.14 | 0.640 |
| 11 | adhE(0002)-cat1(0004)-sucD(0008N) | cat2(0034)-4hbd(0036) | 0.20 | 0.53 | 1.38 | 2.30 | 0.019 | 0.91 | 8.10 | 1.49 | 0.034 |
| 12 | cat1(0004)-sucD(0008N)-adhE(0002) | cat2(0034)-4hbd(0036) | 2.14 | 2.73 | 12.07 | 0.16 | 0.000 | 3.10 | 11.79 | 0.17 | 0.002 |
| 13 | vector only | vector only | 2.11 | 2.62 | 9.03 | 0.01 | 0.000 | 3.00 | 12.05 | 0.01 | 0.000 |

Analysis of BDO, 4-HB and succinate by GCMS. BDO, 4-HB and succinate in fermentation and cell culture samples were derivatized by silylation and quantitatively analyzed by GCMS using methods adapted from literature reports ((Simonov et al., J. Anal. Chem. 59:965-971 (2004)). The developed method demonstrated good sensitivity down to 1 μM, linearity up to at least 25 mM, as well as excellent selectivity and reproducibility.

Sample preparation was performed as follows: 100 μL filtered (0.2 μm or 0.45 μm syringe filters) samples, e.g. fermentation broth, cell culture or standard solutions, were dried down in a Speed Vac Concentrator (Savant SVC-100H) for approximately 1 hour at ambient temperature, followed by the addition of 20 μL 10 mM cyclohexanol solution, as an internal standard, in dimethylformamide. The mixtures were vortexed and sonicated in a water bath (Branson 3510) for 15 min to ensure homogeneity. 100 μL silylation derivatization reagent, N,O-bis(trimethylsilyl)triflouro-acetimide (BSTFA) with 1% trimethylchlorosilane, was added, and the mixture was incubated at 70° C. for 30 min. The derivatized samples were centrifuged for 5 min, and the clear solutions were directly injected into GCMS. All the chemicals and reagents were from Sigma-Aldrich, with the exception of BDO which was purchased from J.T. Baker.

GCMS was performed on an Agilent gas chromatograph 6890N, interfaced to a mass-selective detector (MSD) 5973N operated in electron impact ionization (EI) mode has been used for the analysis. ADB-5MS capillary column (J&W Scientific, Agilent Technologies), 30 m×0.25 mm i.d.×0.25 μm film thickness, was used. The GC was operated in a split injection mode introducing 1 μL of sample at 20:1 split ratio. The injection port temperature was 250° C. Helium was used as a carrier gas, and the flow rate was maintained at 1.0 mL/min. A temperature gradient program was optimized to ensure good resolution of the analytes of interest and minimum matrix interference. The oven was initially held at 80° C. for 1 min, then ramped to 120° C. at 2C/min, followed by fast ramping to 320° C. at 100° C./min and final hold for 6 min at 320° C. The MS interface transfer line was maintained at 280° C. The data were acquired using ‘lowmass’ MS tune settings and 30-400 m/z mass-range scan. The total analysis time was 29 min including 3 min solvent delay. The retention times corresponded to 5.2, 10.5, 14.0 and 18.2 min for BSTFA-derivatized cyclohexanol, BDO, 4-HB and succinate, respectively. For quantitative analysis, the following specific mass fragments were selected (extracted ion chromatograms): m/z 157 for internal standard cyclohexanol, 116 for BDO, and 147 for both 4-HB and succinate. Standard calibration curves were constructed using analyte solutions in the corresponding cell culture or fermentation medium to match sample matrix as close as possible. GCMS data were processed using Environmental Data Analysis ChemStation software (Agilent Technologies).

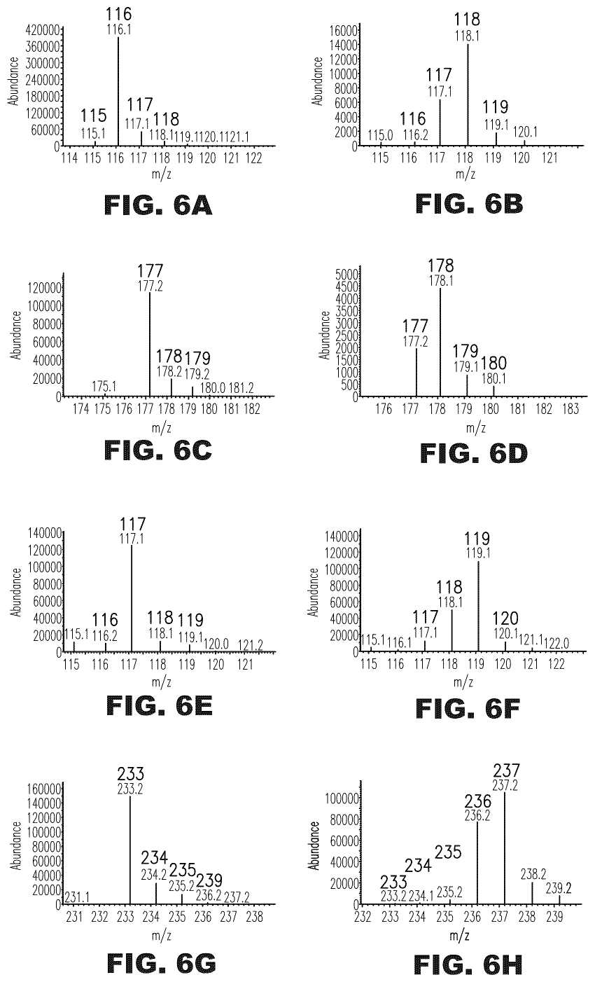

The results indicated that most of the 4-HB and BDO produced were labeled with ¹³C (FIG. 6, right-hand sides). Mass spectra from a parallel culture grown in unlabeled glucose are shown for comparison (FIG. 6, left-hand sides). Note that the peaks seen are for fragments of the derivatized molecule containing different numbers of carbon atoms from the metabolite. The derivatization reagent also contributes some carbon and silicon atoms that naturally-occurring label distribution, so the results are not strictly quantitative.

Production of BDO from 4-HB using alternate pathways. The various alternate pathways were also tested for BDO production. This includes use of the native E. coli SucCD enzyme to convert succinate to succinyl-CoA (Table 13, rows 2-3), use of α-ketoglutarate decarboxylase in the α-ketoglutarate pathway (Table 13, row 4), and use of PTB/BK as an alternate means to generate the CoA-derivative of 4HB (Table 13, row 1). Strains were constructed containing plasmids expressing the genes indicated in Table 13, which encompass these variants. The results show that in all cases, production of 4-HB and BDO occurred (Table 13).

TABLE 13
Production of BDO, 4-HB, and succinate in recombinant E. coli
strains genes for different BDO pathway variants, grown anaerobically
in minimal medium supplemented with 20 g/L glucose, and harvested
24 hours after induction with 0.1 mM IPTG. Concentrations are given
in mM.

| Genes on pZE13 | Genes on pZA33 | Succinate | 4-HB | BDO |
|---|---|---|---|---|
| 0002 + 0004 + 0035 | 0020n-0021n-0036 | 0.336 | 2.91 | 0.230 |
| 0038 + 0035 | 0034-0036 | 0.814 | 2.81 | 0.126 |
| 0038 + 0035 | 0036-0034 | 0.741 | 2.57 | 0.114 |
| 0035 + 0032 | 0034-0036 | 5.01 | 0.538 | 0.154 |

### Example III

### Biosynthesis of 4-Hydroxybutanoic Acid, γ-Butyrolactone and 1,4-Butanediol

This Example describes the biosynthetic production of 4-hydroxybutanoic acid, γ-butyrolactone and 1,4-butanediol using fermentation and other bioprocesses. See corresponding Example in WO2013/184602.

### Example IV

### Exemplary BDO Pathways

This example describes exemplary enzymes and corresponding genes for 1,4-butandiol (BDO) synthetic pathways.

Exemplary BDO synthetic pathways are shown in FIGS. 8-13. The pathways depicted in FIGS. 8-13 are from common central metabolic intermediates to 1,4-butanediol. All transformations depicted in FIGS. 8-13 fall into the 18 general categories of transformations shown in Table 14. Below is described a number of biochemically characterized candidate genes in each category, as described in this and further Examples provided below. Thus, it is understood that a gene for carrying out a specified reaction can be utilized based on any of the disclosures herein, including various Examples, for the same or similar type of reaction. Specifically listed are genes that can be applied to catalyze the appropriate transformations in FIGS. 9-13 when cloned and expressed in a host organism. The top three exemplary genes for each of the key steps in FIGS. 9-13 are described in Tables 15 and 16 herein and in Tables 17-23 in WO2013/184602. Exemplary genes were provided for the pathways depicted in FIG. 8 are described herein.

TABLE 14
Enzyme types required to convert common central metabolic
intermediates into 1,4-butanediol. The first three digits of each
label correspond to the first three Enzyme Commission number
digits which denote the general type of transformation independent
of substrate specificity.

| Label | Function |
|---|---|
| 1.1.1.a | Oxidoreductase (ketone to hydroxyl or aldehyde to alcohol) |
| 1.1.1.c | Oxidoreductase (2 step, acyl-CoA to alcohol) |
| 1.2.1.b | Oxidoreductase (acyl-CoA to aldehyde) |
| 1.2.1.c | Oxidoreductase (2-oxo acid to acyl-CoA, decarboxylation) |
| 1.2.1.d | Oxidoreductase (phosphorylating/dephosphorylating) |
| 1.3.1.a | Oxidoreductase operating on CH—CH donors |
| 1.4.1.a | Oxidoreductase operating on amino acids |
| 2.3.1.a | Acyltransferase (transferring phosphate group) |
| 2.6.1.a | Aminotransferase |
| 2.7.2.a | Phosphotransferase, carboxyl group acceptor |
| 2.8.3.a | Coenzyme-A transferase |
| 3.1.2.a | Thiolester hydrolase (CoA specific) |
| 4.1.1.a | Carboxy-lyase |
| 4.2.1.a | Hydro-lyase |
| 4.3.1.a | Ammonia-lyase |
| 5.3.3.a | Isomerase |
| 5.4.3.a | Aminomutase |
| 6.2.1.a | Acid-thiol ligase |

1.1.1.a—Oxidoreductase (Aldehyde to Alcohol or Ketone to Hydroxyl)

Aldehyde to alcohol. Exemplary genes encoding enzymes that catalyze the conversion of an aldehyde to alcohol, that is, alcohol dehydrogenase or equivalently aldehyde reductase, include alrA encoding a medium-chain alcohol dehydrogenase for C2-C14 (Tani et al. Appl. Environ. Microbiol. 66:5231-5235 (2000)), ADH2 from Saccharomyces cerevisiae (Atsumi et al. Nature 451:86-89 (2008)), yqhD from E. coli which has preference for molecules longer than C(3)(Sulzenbacher et al. Journal of Molecular Biology 342:489-502 (2004)), and bdh I and bdh II from C. acetobutylicum which converts butyraldehyde into butanol (Walter et al. Journal of Bacteriology 174:7149-7158 (1992)). The protein sequences for each of these exemplary gene products, if available, can be found using the following GenBank accession numbers:

| Gene | Accession No. | GI No. | Organism |
|---|---|---|---|
| alrA | BAB12273.1 | 9967138 | Acinetobacter sp. Strain M-1 |
| ADH2 | NP_014032.1 | 6323961 | Saccharymyces cerevisiae |
| yqhD | NP_417484.1 | 16130909 | Escherichia coli |
| bdh I | NP_349892.1 | 15896543 | Clostridium acetobutylicum |
| bdh II | NP_349891.1 | 15896542 | Clostridium acetobutylicum |

Enzymes exhibiting 4-hydroxybutyrate dehydrogenase activity (EC 1.1.1.61) also fall into this category. Such enzymes have been characterized in Ralstonia eutropha (Bravo et al. J. Forensic Sci. 49:379-387 (2004), Clostridium kluyveri (Wolff et al. Protein Expr:Purif. 6:206-212 (1995)) and Arabidopsis thaliana (Breitkreuz et al. J. Biol. Chem. 278:41552-41556 (2003)).

| Gene | Accession No. | GI No. | Organism |
|---|---|---|---|
| 4hbd | YP_726053.1 | 113867564 | Ralstonia eutropha H16 |
| 4hbd | L21902.1 | 146348486 | Clostridium kluyveri DSM 555 |
| 4hbd | Q94B07 | 75249805 | Arabidopsis thaliana |

Another exemplary enzyme is 3-hydroxyisobutyrate dehydrogenase which catalyzes the reversible oxidation of 3-hydroxyisobutyrate to methylmalonate semialdehyde. This enzyme participates in valine, leucine and isoleucine degradation and has been identified in bacteria, eukaryotes, and mammals. The enzyme encoded by P84067 from Thermus thermophilus HB8 has been structurally characterized (Lokanath et al. J Mol Biol 352:905-17 (2005)). The reversibility of the human 3-hydroxyisobutyrate dehydrogenase was demonstrated using isotopically-labeled substrate (Manning et al. Biochem J 231:481-484 (1985)). Additional genes encoding this enzyme include 3hidh in Homo sapiens (Hawes et al. Methods Enzymol. 324:218-228 (2000)) and Oryctolagus cuniculus (Chowdhury et al. Biosci. Biotechnol Biochem. 60:2043-2047 (1996); Hawes et al. Methods Enzymol. 324:218-228 (2000)), mmsb in Pseudomonas aeruginosa, and dhat in Pseudomonas putida (Aberhart et al. J Chem. Soc. [Perkin 1] 6:1404-1406 (1979); Chowdhury et al. Biosci. Biotechnol Biochem. 67:438-441 (2003); Chowdhury et al. Biosci. Biotechnol Biochem. 60:2043-2047 (1996)).

| Gene | Accession No. | GI No. | Organism |
|---|---|---|---|
| P84067 | P84067 | 75345323 | Thermus thermophilus |
| mmsb | P28811.1 | 127211 | Pseudomonas aeruginosa |
| dhat | Q59477.1 | 2842618 | Pseudomonas putida |
| 3hidh | P31937.2 | 12643395 | Homo sapiens |
| 3hidh | P32185.1 | 416872 | Oryctolagus cuniculus |

Several 3-hydroxyisobutyrate dehydrogenase enzymes have also been shown to convert malonic semialdehyde to 3-hydroxypropionic acid (3-HP). Three gene candidates exhibiting this activity are mmsB from Pseudomonas aeruginosa PAO1(62), mmsB from Pseudomonas putida KT2440 (Liao et al., US Publication 2005/0221466) and mmsB from Pseudomonas putida E23 (Chowdhury et al., Biosci. Biotechnol. Biochem. 60:2043-2047 (1996)). An enzyme with 3-hydroxybutyrate dehydrogenase activity in Alcaligenes faecalis M3A has also been identified (Gokam et al., U.S. Pat. No. 7,393,676; Liao et al., US Publication No. 2005/0221466). Additional gene candidates from other organisms including Rhodobacter spaeroides can be inferred by sequence similarity.

| Gene | Accession No. | GI No. | Organism |
|---|---|---|---|
| mmsB | AAA25892.1 | 151363 | Pseudomonas aeruginosa |
| mmsB | NP_252259.1 | 15598765 | Pseudomonas aeruginosa PAO1 |
| mmsB | NP_746775.1 | 26991350 | Pseudomonas putida KT2440 |
| mmsB | JC7926 | 60729613 | Pseudomonas putida E23 |
| orfB1 | AAL26884 | 16588720 | Rhodobacter spaeroides |

The conversion of malonic semialdehyde to 3-HP can also be accomplished by two other enzymes: NADH-dependent 3-hydroxypropionate dehydrogenase and NADPH-dependent malonate semialdehyde reductase. An NADH-dependent 3-hydroxypropionate dehydrogenase is thought to participate in beta-alanine biosynthesis pathways from propionate in bacteria and plants (Rathinasabapathi, B. Journal of Plant Pathology 159:671-674 (2002); Stadtman, E. R. J. Am. Chem. Soc. 77:5765-5766 (1955)). This enzyme has not been associated with a gene in any organism to date. NADPH-dependent malonate semialdehyde reductase catalyzes the reverse reaction in autotrophic CO₂-fixing bacteria. Although the enzyme activity has been detected in Metallosphaera sedula, the identity of the gene is not known (Alber et al. J. Bacteriol. 188:8551-8559 (2006)).

Ketone to hydroxyl. There exist several exemplary alcohol dehydrogenases that convert a ketone to a hydroxyl functional group. Two such enzymes from E. coli are encoded by malate dehydrogenase (mdh) and lactate dehydrogenase (ldhA). In addition, lactate dehydrogenase from Ralstonia eutropha has been shown to demonstrate high activities on substrates of various chain lengths such as lactate, 2-oxobutyrate, 2-oxopentanoate and 2-oxoglutarate (Steinbuchel and. Schlegel, Eur. J. Biochem. 130:329-334 (1983)). Conversion of alpha-ketoadipate into alpha-hydroxyadipate can be catalyzed by 2-ketoadipate reductase, an enzyme reported to be found in rat and in human placenta (Suda et al. Arch. Biochem. Biophys. 176:610-620 (1976); Suda et al. Biochem. Biophys. Res. Commun. 77:586-591 (1977)). An additional candidate for this step is the mitochondrial 3-hydroxybutyrate dehydrogenase (bch) from the human heart which has been cloned and characterized (Marks et al. J. Biol. Chem. 267:15459-15463 (1992)). This enzyme is a dehydrogenase that operates on a 3-hydroxyacid. Another exemplary alcohol dehydrogenase converts acetone to isopropanol as was shown in C. beijerinckii (Ismaiel et al. J. Bacteriol. 175:5097-5105 (1993)) and T. brockii (Lamed et al. Biochem. J 195:183-190 (1981); Peretz and Burstein Biochemistry 28:6549-6555 (1989)).

| Gene | Accession No. | GI No. | Organism |
|---|---|---|---|
| mdh | AAC76268.1 | 1789632 | Escherichia coli |
| ldhA | NP_415898.1 | 16129341 | Escherichia coli |
| ldh | YP_725182.1 | 113866693 | Ralstonia eutropha |
| bdh | AAA58352.1 | 177198 | Homo sapiens |
| adh | AAA23199.2 | 60592974 | Clostridium beijerinckii NRRL |
| | | | B593 |
| adh | P14941.1 | 113443 | Thermoanaerobacter brockii |
| | | | HTD4 |

Exemplary 3-hydroxyacyl dehydrogenases which convert acetoacetyl-CoA to 3-hydroxybutyryl-CoA include hbd from C. acetobutylicum (Boynton et al. Journal of Bacteriology 178:3015-3024 (1996)), hbd from C. beijerinckii (Colby et al. Appl Environ. Microbiol 58:3297-3302(1992)), and a number of similar enzymes from Metallosphaera sedula (Berg et al. Archaea. Science 318:1782-1786 (2007)).

| Gene | Accession No. | GI No. | Organism |
|---|---|---|---|
| hbd | NP_349314.1 | 15895965 | Clostridium |
| | | | acetobutylicum |
| hbd | AAM14586.1 | 20162442 | Clostridium beijerinckii |
| Msed_1423 | YP_001191505 | 146304189 | Metallosphaera sedula |
| Msed_0399 | YP_001190500 | 146303184 | Metallosphaera sedula |
| Msed_0389 | YP_001190490 | 146303174 | Metallosphaera sedula |
| Msed_1993 | YP_001192057 | 146304741 | Metallosphaera sedula |

1.1.1.c—Oxidoredutase (2 step, acyl-CoA to Alcohol)

Exemplary 2-step oxidoreductases that convert an acyl-CoA to alcohol include those that transform substrates such as acetyl-CoA to ethanol (for example, adhE from E. coli (Kessler et al. FEBS. Lett. 281:59-63 (1991)) and butyryl-CoA to butanol (for example, adhE2 from C. acetobutylicum (Fontaine et al. J. Bacteriol. 184:821-830 (2002)). In addition to reducing acetyl-CoA to ethanol, the enzyme encoded by adhE in Leuconostoc mesenteroides has been shown to oxide the branched chain compound isobutyraldehyde to isobutyryl-CoA (Kazahaya et al. J. Gen. Appl. Microbiol. 18:43-55 (1972); Koo et al. Biotechnol Lett. 27:505-510 (2005)).

| Gene | Accession No. | GI No. | Organism |
|---|---|---|---|
| adhE | NP_415757.1 | 16129202 | Escherichia coli |
| adhE2 | AAK09379.1 | 12958626 | Clostridium acetobutylicum |
| adhE | AAV66076.1 | 55818563 | Leuconostoc mesenteroides |

Another exemplary enzyme can convert malonyl-CoA to 3-HP. An NADPH-dependent enzyme with this activity has characterized in Chloroflexus aurantiacus where it participates in the 3-hydroxypropionate cycle (Hugler et al., J. Bacteriol. 184:2404-2410 (2002); Strauss and Fuchs, Eur. J. Biochem. 215:633-643 (1993)). This enzyme, with a mass of 300 kDa, is highly substrate-specific and shows little sequence similarity to other known oxidoreductases (Hugler et al., J. Bacteriol. 184:2404-2410 (2002)). No enzymes in other organisms have been shown to catalyze this specific reaction; however there is bioinformatic evidence that other organisms may have similar pathways (Klatt et al., Environ. Microbiol. 9:2067-2078 (2007)). Enzyme candidates in other organisms including Roseiflexus castenholzii, Erythrobacter sp. NAP1 and marine gamma proteobacterium HTCC2080 can be inferred by sequence similarity.

| Gene | Accession No. | GI No. | Organism |
|---|---|---|---|
| mcr | AAS20429.1 | 42561982 | Chloroflexus |
| | | | aurantiacus |
| Rcas_2929 | YP_001433009.1 | 156742880 | Roseiflexus |
| | | | castenholzii |
| NAP1_02720 | ZP_01039179.1 | 85708113 | Erythrobacter |
| | | | sp. NAP1 |
| MGP2080_00535 | ZP_01626393.1 | 119504313 | marine gamma |
| | | | proteobacterium |
| | | | HTCC2080 |

Longer chain acyl-CoA molecules can be reduced by enzymes such as the jojoba (Simmondsia chinensis) FAR which encodes an alcohol-forming fatty acyl-CoA reductase. Its overexpression in E. coli resulted in FAR activity and the accumulation of fatty alcohol (Metz et al. Plant Physiology 122:635-644) 2000)).

| Gene | Accession No. | GI No. | Organism |
|---|---|---|---|
| FAR | AAD38039.1 | 5020215 | Simmondsia chinensis |

1.2.1.b—Oxidoreductase (Acyl-CoA to Aldehyde)

Several acyl-CoA dehydrogenases are capable of reducing an acyl-CoA to its corresponding aldehyde. Exemplary genes that encode such enzymes include the Acinetobacter calcoaceticus acr1 encoding a fatty acyl-CoA reductase (Reiser and Somerville, J. Bacteriology 179:2969-2975 (1997)), the Acinetobacter sp. M-1 fatty acyl-CoA reductase (Ishige et al. Appl. Environ. Microbiol. 68:1192-1195 (2002)), and a CoA- and NADP-dependent succinate semialdehyde dehydrogenase encoded by the sucD gene in Clostridium kluyveri (Sohling and Gottschalk J Bacteriol 178:871-80 (1996); Sohling and Gottschalk J Bacteriol. 178:871-880 (1996)). SucD of P. gingivalis is another succinate semialdehyde dehydrogenase (Takahashi et al. J. Bacteriol. 182:47044710 (2000)). The enzyme acylating acetaldehyde dehydrogenase in Pseudomonas sp, encoded by bphG, is yet another as it has been demonstrated to oxidize and acylate acetaldehyde, propionaldehyde, butyraldehyde, isobutyraldehyde and formaldehyde (Powlowski et al. J Bacteriol. 175:377-385 (1993

| Gene | Accession No. | GI No. | Organism |
|---|---|---|---|
| acr 1 | YP_047869.1 | 50086359 | Acinetobacter calcoaceticus |
| acr 1 | AAC45217 | 1684886 | Acinetobacter baylyi |
| acr 1 | BAB85476.1 | 18857901 | Acinetobacter sp. Strain M-1 |
| sucD | P38947.1 | 730847 | Clostridium kluyveri |
| sucD | NP_904963.1 | 34540484 | Porphyromonas gingivalis |
| bphG | BAA03892.1 | 425213 | Pseudomonas sp |

An additional enzyme type that converts an acyl-CoA to its corresponding aldehyde is malonyl-CoA reductase which transforms malonyl-CoA to malonic semialdehyde. Malonyl-CoA reductase is a key enzyme in autotrophic carbon fixation via the 3-hydroxypropionate cycle in thermoacidophilic archael bacteria (Berg et al. Science 318:1782-1786 (2007); Thauer, R. K. Science 318:1732-1733 (2007)). The enzyme utilizes NADPH as a cofactor and has been characterized in Metallosphaera and Sulfolobus spp (Alber et al. J. Bacteriol. 188:8551-8559 (2006); Hugler et al. J. Bacteriol. 184:2404-2410 (2002)). The enzyme is encoded by Msed_0709 in Metallosphaera sedula (Alber et al. J. Bacteriol. 188:8551-8559 (2006); Berg et al. Science 318:1782-1786(2007)). Agene encoding a malonyl-CoA reductase from Sulfolobus tokodaii was cloned and heterologously expressed in E. coli (Alber et al. J. Bacteriol. 188:8551-8559 (2006)). Although the aldehyde dehydrogenase functionality of these enzymes is similar to the bifunctional dehydrogenase from Chloroflexus aurantiacus, there is little sequence similarity. Both malonyl-CoA reductase enzyme candidates have high sequence similarity to aspartate-semialdehyde dehydrogenase, an enzyme catalyzing the reduction and concurrent dephosphorylation of aspartyl-4-phosphate to aspartate semialdehyde. Additional gene candidates can be found by sequence homology to proteins in other organisms including Sulfolobus solfataricus and Sulfolobus acidocaldarius.

| Gene | Accession No. | GI No. | Organism |
|---|---|---|---|
| Msed_0709 | YP_001190808.1 | 146303492 | Metallosphaera sedula |
| mcr | NP_378167.1 | 15922498 | Sulfolobus tokodaii |
| asd-2 | NP_343563.1 | 15898958 | Sulfolobus solfataricus |
| Saci_2370 | YP_256941.1 | 70608071 | Sulfolobus |
| | | | acidocaldarius |

1.2.1.c—Oxidoreductase (2-Oxo Acid to Acyl-CoA, Decarboxylation)

Enzymes in this family include 1) branched-chain 2-keto-acid dehydrogenase, 2) alpha-ketoglutarate dehydrogenase, and 3) the pyruvate dehydrogenase multienzyme complex (PDHC). These enzymes are multi-enzyme complexes that catalyze a series of partial reactions which result in acylating oxidative decarboxylation of 2-keto-acids. Each of the 2-keto-acid dehydrogenase complexes occupies key positions in intermediary metabolism, and enzyme activity is typically tightly regulated (Fries et al. Biochemistry 42:6996-7002 (2003)). The enzymes share a complex but common structure composed of multiple copies of three catalytic components: alpha-ketoacid decarboxylase (E1), dihydrolipoamide acyltransferase (E2) and dihydrolipoamide dehydrogenase (E3). The E3 component is shared among all 2-keto-acid dehydrogenase complexes in an organism, while the E1 and E2 components are encoded by different genes. The enzyme components are present in numerous copies in the complex and utilize multiple cofactors to catalyze a directed sequence of reactions via substrate channeling. The overall size of these dehydrogenase complexes is very large, with molecular masses between 4 and 10 million Da (that is, larger than a ribosome).

Activity of enzymes in the 2-keto-acid dehydrogenase family is normally low or limited under anaerobic conditions in E. coli. Increased production of NADH (or NADPH) could lead to a redox-imbalance, and NADH itself serves as an inhibitor to enzyme function. Engineering efforts have increased the anaerobic activity of the E. coli pyruvate dehydrogenase complex (Kim et al. Appl. Environ. Microbiol. 73:1766-1771 (2007); Kim et al. J. Bacteriol. 190:3851-3858)2008); Zhou et al. Biotechnol. Lett. 30:335-342(2008)). For example, the inhibitory effect of NADH can be overcome by engineering an H322Y mutation in the E3 component (Kim et al. J. Bacteriol. 190:3851-3858 (2008)). Structural studies of individual components and how they work together in complex provide insight into the catalytic mechanisms and architecture of enzymes in this family (Aevarsson et al. Nat. Struct. Biol. 6:785-792 (1999); Zhou et al. Proc. Natl. Acad. Sci. U.S.A. 98:14802-14807 (2001)). The substrate specificity of the dehydrogenase complexes varies in different organisms, but generally branched-chain keto-acid dehydrogenases have the broadest substrate range.

Alpha-ketoglutarate dehydrogenase (AKGD) converts alpha-ketoglutarate to succinyl-CoA and is the primary site of control of metabolic flux through the TCA cycle (Hansford, R. G. Curr. Top. Bioenerg. 10:217-278 (1980)). Encoded by genes sucA, sucB and Ipd in E. coli, AKGD gene expression is downregulated under anaerobic conditions and during growth on glucose (Park et al. Mol. Microbiol. 15:473-482(1995)). Although the substrate range of AKGD is narrow, structural studies of the catalytic core of the E2 component pinpoint specific residues responsible for substrate specificity (Knapp et al. J. Mol. Biol. 280:655-668 (1998)). The Bacillus subtilis AKGD, encoded by odhAB (E1 and E2) and pdhD (E3, shared domain), is regulated at the transcriptional level and is dependent on the carbon source and growth phase of the organism (Resnekov et al. Mol. Gen. Genet. 234:285-296 (1992)). In yeast, the LPD1 gene encoding the E3 component is regulated at the transcriptional level by glucose (Roy and Dawes J. Gen. Microbiol. 133:925-933 (1987)). The E1 component, encoded by KGD1, is also regulated by glucose and activated by the products of HAP2 and HAP3 (Repetto and Tzagoloff Mol. Cell Biol. 9:2695-2705 (1989)). The AKGD enzyme complex, inhibited by products NADH and succinyl-CoA, is well-studied in mammalian systems, as impaired function of has been linked to several neurological diseases (Tretter and dam-Vizi Philos. Trans. R. Soc. Lond B Biol. Sci. 360:2335-2345 (2005)).

| Gene | Accession No. | GI No. | Organism |
|---|---|---|---|
| sucA | NP_415254.1 | 16128701 | Escherichia coli str. K12 substr. |
| | | | MG1655 |
| sucB | NP_415255.1 | 16128702 | Escherichia coli str. K12 substr. |
| | | | MG1655 |
| lpd | NP_414658.1 | 16128109 | Escherichia coli str. K12 substr. |
| | | | MG1655 |
| odhA | P23129.2 | 51704265 | Bacillus subtilis |
| odhB | P16263.1 | 129041 | Bacillus subtilis |
| pdhD | P21880.1 | 118672 | Bacillus subtilis |
| KGD1 | NP_012141.1 | 6322066 | Saccharomyces cerevisiae |
| KGD2 | NP_010432.1 | 6320352 | Saccharomyces cerevisiae |
| LPD1 | NP_116635.1 | 14318501 | Saccharomyces cerevisiae |

Branched-chain 2-keto-acid dehydrogenase complex (BCKAD), also known as 2-oxoisovalerate dehydrogenase, participates in branched-chain amino acid degradation pathways, converting 2-keto acids derivatives of valine, leucine and isoleucine to their acyl-CoA derivatives and CO₂. The complex has been studied in many organisms including Bacillus subtilis (Wang et al. Eur. J. Biochem. 213:1091-1099 (1993)), Rattus norvegicus (Namba et al. J. Biol. Chem. 244:4437-4447 (1969)) and Pseudomonas putida (Sokatch J. Bacteriol. 148:647-652 (1981)). In Bacillus subtilis the enzyme is encoded by genes pdhD (E3 component), bfmBB (E2 component), bfmBAA and bfmBAB (E1 component)(Wang et al. Eur. J. Biochem. 213:1091-1099 (1993)). In mammals, the complex is regulated by phosphorylation by specific phosphatases and protein kinases. The complex has been studied in rat hepatocites (Chicco et al. J. Biol. Chem. 269:19427-19434 (1994)) and is encoded by genes Bckdha (E1 alpha), Bckdhb (E1 beta), Dbt (E2), and Dld (E3). The E1 and E3 components of the Pseudomonas putida BCKAD complex have been crystallized (Aevarsson et al. Nat. Struct. Biol. 6:785-792 (1999); Mattevi Science 255:1544-1550 (1992)) and the enzyme complex has been studied (Sokatch et al. J. Bacteriol. 148:647-652(1981)). Transcription of the P. putida BCKAD genesis activated by the gene product of bkdR (Hester et al. Eur. J. Biochem. 233:828-836 (1995)). In some organisms including Rattus norvegicus (Paxton et al. Biochem. J. 234:295-303 (1986)) and Saccharomyces cerevisiae (Sinclair et al. Biochem. Mol. Biol. Int. 31:911-922(1993)), this complex has been shown to have a broad substrate range that includes linear oxo-acids such as 2-oxobutanoate and alpha-ketoglutarate, in addition to the branched-chain amino acid precursors. The active site of the bovine BCKAD was engineered to favor alternate substrate acetyl-CoA (Meng and Chuang, Biochemistry 33:12879-12885 (1994)).

| Gene | Accession No. | GI No. | Organism |
|---|---|---|---|
| bfmBB | NP_390283.1 | 16079459 | Bacillus subtilis |
| bfmBAA | NP_390285.1 | 16079461 | Bacillus subtilis |
| bfmBAB | NP_390284.1 | 16079460 | Bacillus subtilis |
| pdhD | P21880.1 | 118672 | Bacillus subtilis |
| lpdV | P09063.1 | 118677 | Pseudomonas putida |
| bkdB | P09062.1 | 129044 | Pseudomonas putida |
| bkdA1 | NP_746515.1 | 26991090 | Pseudomonas putida |
| bkdA2 | NP_746516.1 | 26991091 | Pseudomonas putida |
| Bckdha | NP_036914.1 | 77736548 | Rattus norvegicus |
| Bckdhb | NP_062140.1 | 158749538 | Rattus norvegicus |
| Dbt | NP_445764.1 | 158749632 | Rattus norvegicus |
| Dld | NP_955417.1 | 40786469 | Rattus norvegicus |

The pyruvate dehydrogenase complex, catalyzing the conversion of pyruvate to acetyl-CoA, has also been extensively studied. In the E. coli enzyme, specific residues in the E1 component are responsible for substrate specificity (Bisswanger, H. J Biol Chem. 256:815-822(1981); Bremer, J. Eur. J Biochem. 8:535-540 (1969); Gong et al. J Biol Chem. 275:13645-13653 (2000)). As mentioned previously, enzyme engineering efforts have improved the E. coli PDH enzyme activity under anaerobic conditions (Kim et al. Appl. Environ. Microbiol. 73:1766-1771(2007); Kim J. Bacteriol. 190:3851-3858 (2008); Zhou et al. Biotechnol. Lett. 30:335-342 (2008)). In contrast to the E. coli PDH, the B. subtilis complex is active and required for growth under anaerobic conditions (Nakano J. Bacteriol. 179:6749-6755 (1997)). The Klebsiella pneumoniae PDH, characterized during growth on glycerol, is also active under anaerobic conditions (Menzel et al. J. Biotechnol. 56:135-142 (1997)). Crystal structures of the enzyme complex from bovine kidney (Zhou et al. Proc. Natl. Acad. Sci. U.S.A. 98:14802-14807 (2001)) and the E2 catalytic domain from Azotobacter vinelandii are available (Mattevi et al. Science 255:1544-1550 (1992)). Some mammalian PDH enzymes complexes can react on alternate substrates such as 2-oxobutanoate, although comparative kinetics of Rattus norvegicus PDH and BCKAD indicate that BCKAD has higher activity on 2-oxobutanoate as a substrate (Paxton et al. Biochem. J. 234:295-303 (1986)).

| Gene | Accession No. | GI No. | Organism |
|---|---|---|---|
| aceE | NP_414656.1 | 16128107 | Escherichia coli str. K12 |
| | | | substr. MG1655 |
| aceF | NP_414657.1 | 16128108 | Escherichia coli str. K12 |
| | | | substr. MG1655 |
| lpd | NP_414658.1 | 16128109 | Escherichia coli str. K12 |
| | | | substr. MG1655 |
| pdhA | P21881.1 | 3123238 | Bacillus subtilis |
| pdhB | P21882.1 | 129068 | Bacillus subtilis |
| pdhC | P21883.2 | 129054 | Bacillus subtilis |
| pdhD | P21880.1 | 118672 | Bacillus subtilis |
| aceE | YP_001333808.1 | 152968699 | Klebsiella pneumonia |
| | | | MGH78578 |
| aceF | YP_001333809.1 | 152968700 | Klebsiella pneumonia |
| | | | MGH78578 |
| lpdA | YP_001333810.1 | 152968701 | Klebsiella pneumonia |
| | | | MGH78578 |
| Pdha1 | NP_001004072.2 | 124430510 | Rattus norvegicus |
| Pdha2 | NP_446446.1 | 16758900 | Rattus norvegicus |
| Dlat | NP_112287.1 | 78365255 | Rattus norvegicus |
| Dld | NP_955417.1 | 40786469 | Rattus norvegicus |

As an alternative to the large multienzyme 2-keto-acid dehydrogenase complexes described above, some anaerobic organisms utilize enzymes in the 2-ketoacid oxidoreductase family (OFOR) to catalyze acylating oxidative decarboxylation of 2-keto-acids. Unlike the dehydrogenase complexes, these enzymes contain iron-sulfur clusters, utilize different cofactors, and use ferredoxin or flavodixin as electron acceptors in lieu of NAD(P)H. While most enzymes in this family are specific to pyruvate as a substrate (POR) some 2-keto-acid:ferredoxin oxidoreductases have been shown to accept a broad range of 2-ketoacids as substrates including alpha-ketoglutarate and 2-oxobutanoate (Fukuda and Wakagi Biochim. Biophys. Acta 1597:74-80 (2002); Zhang et al. J. Biochem. 120:587-599 (1996)). One such enzyme is the OFOR from the thermoacidophilic archaeon Sulfolobus tokodaii 7, which contains an alpha and beta subunit encoded by gene ST2300 (Fukuda and Wakagi Biochim. Biophys. Acta 1597:74-80 (2002); Zhang et al. J. Biochem. 120:587-599 (1996)). A plasmid-based expression system has been developed for efficiently expressing this protein in E. coli (Fukuda et al. Eur. J. Biochem. 268:5639-5646 (2001)) and residues involved in substrate specificity were determined (Fukuda and Wakagi Biochim. Biophys. Acta 1597:74-80 (2002)). Two OFORs from Aeropyrum pernix str. K1 have also been recently cloned into E. coli, characterized, and found to react with a broad range of 2-oxoacids (Nishizawa et al. FEBS Lett. 579:2319-2322 (2005)). The gene sequences of these OFOR candidates are available, although they do not have GenBank identifiers assigned to date. There is bioinformatic evidence that similar enzymes are present in all archaea, some anaerobic bacteria and a mitochondrial eukarya (Fukuda and Wakagi Biochim. Biophys. Acta 1597:74-80 (2005)). This class of enzyme is also interesting from an energetic standpoint, as reduced ferredoxin could be used to generate NADH by ferredoxin-NAD reductase (Petitdemange et al. Biochim. Biophys. Acta 421:334-337 (1976)). Also, since most of the enzymes are designed to operate under anaerobic conditions, less enzyme engineering may be required relative to enzymes in the 2-keto-acid dehydrogenase complex family for activity in an anaerobic environment.

| Gene | Accession No. | GI No. | Organism |
|---|---|---|---|
| ST2300 | NP_378302.1 | 15922633 | Sulfolobus tokodaii 7 |

1.2.1.d—Oxidoreductase (Phosphorylating/Dephosphorylating)

Exemplary enzymes in this class include glyceraldehyde 3-phosphate dehydrogenase which converts glyceraldehyde-3-phosphate into D-glycerate 1,3-bisphosphate (for example, E. coli gapA (Branlant and Branlant Eur. J. Biochem. 150:61-66(1985)), aspartate-semialdehyde dehydrogenase which converts L-aspartate-4-semialdehyde into L-4-aspartyl-phosphate (for example, E. coli asd (Biellmann et al. Eur. J. Biochem. 104:53-58 (1980)), N-acetyl-gamma-glutamyl-phosphate reductase which converts N-acetyl-L-glutamate-5-semialdehyde into N-acetyl-L-glutamyl-5-phosphate (for example, E. coli argC (Parsot et al. Gene 68:275-283 (1988)), and glutamate-5-semialdehyde dehydrogenase which converts L-glutamate-5-semialdehyde into L-glutamyl-5-phospate (for example, E. coli proA (Smith et al. J. Bacteriol. 157:545-551 (1984)).

| Gene | Accession No. | GI No. | Organism |
|---|---|---|---|
| gapA | P0A9B2.2 | 71159358 | Escherichia coli |
| asd | NP_417891.1 | 16131307 | Escherichia coli |
| argC | NP_418393.1 | 16131796 | Escherichia coli |
| proA | NP_414778.1 | 16128229 | Escherichia coli |

1.3.1.a—Oxidoreductase Operating on CH—CH Donors

An exemplary enoyl-CoA reductase is the gene product of bcd from C. acetobutylicum (Atsumi et al. Metab Eng (2007); Boynton et al. Journal of Bacteriology 178:3015-3024 (1996), which naturally catalyzes the reduction of crotonyl-CoA to butyryl-CoA. Activity of this enzyme can be enhanced by expressing bcd in conjunction with expression of the C. acetobutylicum etfAB genes, which encode an electron transfer flavoprotein. An additional candidate for the enoyl-CoA reductase step is the mitochondrial enoyl-CoA reductase from E. gracilis (Hoffmeister et al. Journal of Biological Chemistry 280:4329-4338 (2005)). A construct derived from this sequence following the removal of its mitochondrial targeting leader sequence was cloned in E. coli resulting in an active enzyme (Hoffmeister et al., supra, (2005)). This approach is well known to those skilled in the art of expressing eukaryotic genes, particularly those with leader sequences that may target the gene product to a specific intracellular compartment, in prokaryotic organisms. A close homolog of this gene, TDE0597, from the prokaryote Treponema denticola represents a third enoyl-CoA reductase which has been cloned and expressed in E. coli (Tucci and Martin FEBS Letters 581:1561-1566 (2007)).

| Gene | Accession No. | GI No. | Organism |
|---|---|---|---|
| bcd | NP_349317.1 | 15895968 | Clostridium acetobutylicum |
| etfA | NP_349315.1 | 15895966 | Clostridium acetobutylicum |
| etfB | NP_349316.1 | 15895967 | Clostridium acetobutylicum |
| TER | Q5EU90.1 | 62287512 | Euglena gracilis |
| TDE0597 | NP_971211.1 | 42526113 | Treponema denticola |

Exemplary 2-enoate reductase (EC 1.3.1.31) enzymes are known to catalyze the NADH-dependent reduction of a wide variety of α, β-unsaturated carboxylic acids and aldehydes (Rohdich et al. J. Biol. Chem. 276:5779-5787 (2001)). 2-Enoate reductase is encoded by enr in several species of Clostridia (Giesel and Simon Arch Microbiol. 135(1): p. 51-57 (2001) including C. tyrobutyricum, and C. thermoaceticum (now called Moorella thermoaceticum)(Rohdich et al., supra, (2001)). In the recently published genome sequence of C. kluyveri, 9 coding sequences for enoate reductases have been reported, out of which one has been characterized (Seedorf et al. Proc Natl Acad Sci U.S.A 105(6):2128-33 (2008)). The enr genes from both C. tyrobutyricum and C. thermoaceticum have been cloned and sequenced and show 59% identity to each other. The former gene is also found to have approximately 75% similarity to the characterized gene in C. kluyveri (Giesel and Simon Arch Microbiol 135(1):51-57 (1983)). It has been reported based on these sequence results that enr is very similar to the dienoyl CoA reductase in E. coli (fadH)(163 Rohdich et al., supra (2001)). The C. thermoaceticum enr gene has also been expressed in an enzymatically active form in E. coli(163 Rohdich et al., supra (2001)).

| Gene | Accession No. | GI No. | Organism |
|---|---|---|---|
| fadH | NP_417552.1 | 16130976 | Escherichia coli |
| enr | ACA54153.1 | 169405742 | Clostridium botulinum A3 str |
| enr | CAA71086.1 | 2765041 | Clostridium tyrobutyricum |
| enr | CAA76083.1 | 3402834 | Clostridium kluyveri |
| enr | YP_430895.1 | 83590886 | Moorella thermoacetica |

1.4.1.a—Oxidoreductase Operating on Amino Acids

Most oxidoreductases operating on amino acids catalyze the oxidative deamination of alpha-amino acids with NAD+ or NADP+ as acceptor. Exemplary oxidoreductases operating on amino acids include glutamate dehydrogenase (deaminating), encoded by gdA, leucine dehydrogenase (deaminating), encoded by ldh, and aspartate dehydrogenase (deaminating), encoded by nadX The gdhA gene product from Escherichia coli (Korber et al. J. Mol. Biol. 234:1270-1273 (1993); McPherson and Wootton Nucleic. Acids Res. 11:5257-5266 (1983)), gdh from Thermotoga maritima (Kort et al. Extremophiles 1:52-60 (1997); Lebbink, et al. J. Mol. Biol. 280:287-296 (1998)); Lebbink et al. J. Mol. Biol. 289:357-369 (1999)), and gdhA1 from Halobacterium salinarum (Ingoldsby et al. Gene 349:237-244 (2005)) catalyze the reversible interconversion of glutamate to 2-oxoglutarate and ammonia, while favoring NADP(H), NAD(H), or both, respectively. The ldh gene of Bacillus cereus encodes the LeuDH protein that has a wide of range of substrates including leucine, isoleucine, valine, and 2-aminobutanoate (Ansorge and Kula Biotechnol Bioeng. 68:557-562 (2000); Stoyan et al. J. Biotechnol 54:77-80 (1997)). The nadX gene from Thermotoga maritime encoding for the aspartate dehydrogenase is involved in the biosynthesis of NAD (Yang et al. J. Biol. Chem. 278:8804-8808 (2003)).

| Gene | Accession No. | GI No. | Organism |
|---|---|---|---|
| gdhA | P00370 | 118547 | Escherichia coli |
| gdh | P96110.4 | 6226595 | Thermotoga maritima |
| gdhA1 | NP_279651.1 | 15789827 | Halobacterium salinarum |
| ldh | P0A393 | 61222614 | Bacillus cereus |
| nadX | NP_229443.1 | 15644391 | Thermotoga maritima |

The lysine 6-dehydrogenase (deaminating), encoded by lysDH gene, catalyze the oxidative deamination of the ε-amino group of L-lysine to form 2-aminoadipate-6-semialdehyde, which in turn nonenzymatically cyclizes to form Δ1-piperidine-6-carboxylate (Misono and Nagasaki J. Bacteriol. 150:398-401 (1982)). The lysDH gene from Geobacillus stearothermophilus encodes a thermophilic NAD-dependent lysine 6-dehydrogenase (Heydari et al. Appl Environ. Microbiol 70:937-942 (2004)). In addition, the lysDH gene from Aeropyrum pernix K1 is identified through homology from genome projects.

| Gene | Accession No. | GI No. | Organism |
|---|---|---|---|
| lysDH | AB052732 | 13429872 | Geobacillus stearothermophilus |
| lysDH | NP_147035.1 | 14602185 | Aeropyrum pernix K1 |
| ldh | P0A393 | 61222614 | Bacillus cereus |

2.3.1.a—Acyltransferase (Transferring Phosphate Group)

Exemplary phosphate transferring acyltransferases include phosphotransacetylase, encoded by pta, and phosphotransbutyrylase, encoded by ptb. The pta gene from E. coli encodes an enzyme that can convert acetyl-CoA into acetyl-phosphate, and vice versa (Suzuki, T. Biochim. Biophys. Acta 191:559-569 (1969)). This enzyme can also utilize propionyl-CoA instead of acetyl-CoA forming propionate in the process (Hesslinger et al. Mol. Microbiol 27:477492 (1998)). Similarly, the ptb gene from C. acetobutylicum encodes an enzyme that can convert butyryl-CoA into butyryl-phosphate (Walter et al. Gene 134(1): p. 107-11 (1993)); Huang et al. J Mol Microbiol Biotechnol 2(1): p. 33-38 (2000). Additional ptb genes can be found in butyrate-producing bacterium L2-50 (Louis et al. J. Bacteriol. 186:2099-2106 (2004)) and Bacillus megaterium (Vazquez et al. Curr. Microbiol 42:345-349 (2001)).

| Gene | Accession No. | GI No. | Organism |
|---|---|---|---|
| pta | NP_416800.1 | 16130232 | Escherichia coli |
| ptb | NP_349676 | 15896327 | Clostridium acetobutylicum |
| ptb | AAR19757.1 | 38425288 | butyrate-producing bacterium L2-50 |
| ptb | CAC07932.1 | 10046659 | Bacillus megaterium |

2.6.1.a—Aminotransferase

Aspartate aminotransferase transfers an amino group from aspartate to alpha-ketoglutarate, forming glutamate and oxaloacetate. This conversion is catalyzed by, for example, the gene products of aspC from Escherichia coli (Yagi et al. FEBS Lett. 100:81-84 (1979); Yagi et al. Methods Enzymol. 113:83-89 (1985)), AAT2 from Saccharomyces cerevisiae (Yagi et al. J Biochem. 92:3543 (1982)) and ASP5 from Arabidopsis thaliana (48, 108, 225 48. de la et al. Plant J 46:414425 (2006); Kwok and Hanson J Exp. Bot. 55:595-604 (2004); Wilkie and Warren Protein Expr. Purif. 12:381-389(1998)). Valine aminotransferase catalyzes the conversion of valine and pyruvate to 2-ketoisovalerate and alanine. The E. coli gene, avtA, encodes one such enzyme (Whalen and Berg J. Bacteriol. 150:739-746 (1982)). This gene product also catalyzes the amination of α-ketobutyrate to generate α-aminobutyrate, although the amine donor in this reaction has not been identified (Whalen and Berg J. Bacteriol. 158:571-574 (1984)). The gene product of the E. coli serC catalyzes two reactions, phosphoserine aminotransferase and phosphohydroxythreonine aminotransferase (Lam and Winkler J. Bacteriol. 172:6518-6528 (1990)), and activity on non-phosphorylated substrates could not be detected (Drewke et al. FEBS. Lett. 390:179-182 (1996)).

| Gene | Accession No. | GI No. | Organism |
|---|---|---|---|
| aspC | NP_415448.1 | 16128895 | Escherichia coli |
| AAT2 | P23542.3 | 1703040 | Saccharomyces cerevisiae |
| ASP5 | P46248.2 | 20532373 | Arabidopsis thaliana |
| avtA | YP_026231.1 | 49176374 | Escherichia coli |
| serC | NP_415427.1 | 16128874 | Escherichia coli |

Cargill has developed a beta-alanine/alpha-ketoglutarate aminotransferase for producing 3-HP from beta-alanine via malonyl-semialdehyde (PCT/US2007/076252 (Jessen et al)). The gene product of SkPYD4 in Saccharomyces kluyveri was also shown to preferentially use beta-alanine as the amino group donor (Andersen et al. FEBS. J. 274:1804-1817 (2007)). SkUGA1 encodes a homologue of Saccharomyces cerevisiae GABA aminotransferase, UGA1 (Ramos et al. Eur. J. Biochem. 149:401-404 (1985)), whereas SkPYD4 encodes an enzyme involved in both β-alanine and GABA transamination (Andersen et al. FEBS. J. 274:1804-1817 (2007)). 3-Amino-2-methylpropionate transaminase catalyzes the transformation from methylmalonate semialdehyde to 3-amino-2-methylpropionate. The enzyme has been characterized in Rattus norvegicus and Sus scrofa and is encoded by Abat (Kakimoto et al. Biochim. Biophys. Acta 156:374-380 (1968); Tamaki et al. Methods Enzymol. 324:376-389 (2000)). Enzyme candidates in other organisms with high sequence homology to 3-amino-2-methylpropionate transaminase include Gta-1 in C. elegans and gabT in Bacillus subtilus. Additionally, one of the native GABA aminotransferases in E. coli, encoded by gene gabT, has been shown to have broad substrate specificity (Liu et al. Biochemistry 43:10896-10905 (2004); Schulz et al. Appl Environ Microbiol 56:1-6 (1990)). The gene product of puuE catalyzes the other 4-aminobutyrate transaminase in E. coli (Kurihara et al. J. Biol. Chem. 280:46024608 (2005)).

| Gene | Accession No. | GI No. | Organism |
|---|---|---|---|
| SkyPYD4 | ABF58893.1 | 98626772 | Saccharomyces kluyveri |
| SkUGA1 | ABF58894.1 | 98626792 | Saccharomyces kluyveri |
| UGA1 | NP_011533.1 | 6321456 | Saccharomyces cerevisiae |
| Abat | P50554.3 | 122065191 | Rattus norvegicus |
| Abat | P80147.2 | 120968 | Sus scrofa |
| Gta-1 | Q21217.1 | 6016091 | Caenorhabditis elegans |
| gabT | P94427.1 | 6016090 | Bacillus subtilus |
| gabT | P22256.1 | 120779 | Escherichia coli K12 |
| puuE | NP_415818.1 | 16129263 | Escherichia coli K12 |

The X-ray crystal structures of E. coli 4-aminobutyrate transaminase unbound and bound to the inhibitor were reported (Liu et al. Biochemistry 43:10896-10905 (2004)). The substrates binding and substrate specificities were studied and suggested. The roles of active site residues were studied by site-directed mutagenesis and X-ray crystallography (Liu et al. Biochemistry 44:2982-2992(2005)). Based on the structural information, attempt was made to engineer E. coli 4-aminobutyrate transaminase with novel enzymatic activity. These studies provide a base for evolving transaminase activity for BDO pathways.

2.7.2.a—Phosphotransferase, Carboxyl Group Acceptor

Exemplary kinases include the E. coli acetate kinase, encoded by ackA (Skarstedt and Silverstein J. Biol. Chem. 251:6775-6783 (1976)), the C. acetobutylicum butyrate kinases, encoded by buk1 and buk2 (Walter et al. Gene 134(1):107-111 (1993) (Huang et al. J Mol Microbiol Biotechnol 2(1):33-38 (2000)], and the E. coli gamma-glutamyl kinase, encoded by proB (Smith et al. J. Bacteriol. 157:545-551 (1984)). These enzymes phosphorylate acetate, butyrate, and glutamate, respectively. The ackA gene product from E. coli also phosphorylates propionate (Hesslinger et al. Mol. Microbiol 27:477-492(1998)).

| Gene | Accession No. | GI No. | Organism |
|---|---|---|---|
| ackA | NP_416799.1 | 16130231 | Escherichia coli |
| buk1 | NP_349675 | 15896326 | Clostridium acetobutylicum |
| buk2 | Q97II1 | 20137415 | Clostridium acetobutylicum |
| proB | NP_414777.1 | 16128228 | Escherichia coli |

2.8.3.a—Coenzyme-A Transferase

In the CoA-transferase family, E. coli enzyme acyl-CoA:acetate-CoA transferase, also known as acetate-CoA transferase (EC 2.8.3.8), has been shown to transfer the CoA moiety to acetate from a variety of branched and linear acyl-CoA substrates, including isobutyrate (Matthies and Schink Appl Environ Microbiol 58:1435-1439 (1992)), valerate (Vanderwinkel et al. Biochem. Biophys. Res Commun. 33:902-908 (1968)) and butanoate (Vanderwinkel, supra (1968)). This enzyme is encoded by atoA (alpha subunit) and atoD (beta subunit) in E. coli sp. K12 (Korolev et al. Acta Crystallogr. D Biol Crystallogr. 58:2116-2121 (2002); Vanderwinkel, supra (1968)) and actA and cg0592 in Corynebacterium glutamicum ATCC13032 (Duncan et al. Appl Environ Microbiol 68:5186-5190 (2002)). Additional genes found by sequence homology include atoD and atoA in Escherichia coli UT189.

| Gene | Accession No. | GI No. | Organism |
|---|---|---|---|
| atoA | P76459.1 | 2492994 | Escherichia coli K12 |
| atoD | P76458.1 | 2492990 | Escherichia coli K12 |
| actA | YP_226809.1 | 62391407 | Corynebacterium glutamicum |
| | | | ATCC 13032 |
| cg0592 | YP_224801.1 | 62389399 | Corynebacterium glutamicum |
| | | | ATCC 13032 |
| atoA | ABE07971.1 | 91073090 | Escherichia coli UT189 |
| atoD | ABE07970.1 | 91073089 | Escherichia coli UT189 |

Similar transformations are catalyzed by the gene products of cat1, cat2, and cat3 of Clostridium kluyveri which have been shown to exhibit succinyl-CoA, 4-hydroxybutyryl-CoA, and butyryl-CoA acetyltransferase activity, respectively (Seedorf et al. Proc Natl Acad Sci U.S.A. 105(6):2128-2133 (2008); Sohling and Gottschalk J Bacteriol 178(3):871-880 (1996)].

| Gene | Accession No. | GI No. | Organism |
|---|---|---|---|
| cat1 | P38946.1 | 729048 | Clostridium kluyveri |
| cat2 | P38942.2 | 1705614 | Clostridium kluyveri |
| cat3 | EDK35586.1 | 146349050 | Clostridium kluyveri |

The glutaconate-CoA-transferase (EC 2.8.3.12) enzyme from anaerobic bacterium Acidaminococcus fermentans reacts with diacid glutaconyl-CoA and 3-butenoyl-CoA (Mack and Buckel FEBS Lett. 405:209-212(1997)). The genes encoding this enzyme are gctA and gctB. This enzyme has reduced but detectable activity with other CoA derivatives including glutaryl-CoA, 2-hydroxyglutaryl-CoA, adipyl-CoA and acrylyl-CoA (Buckel et al. Eur. J. Biochem. 118:315-321(1981)). The enzyme has been cloned and expressed in E. coli (Mac et al. Eur. J. Biochem. 226:41-51 (1994)).

| Gene | Accession No. | GI No. | Organism |
|---|---|---|---|
| gctA | CAA57199.1 | 559392 | Acidaminococcus fermentans |
| gctB | CAA57200.1 | 559393 | Acidaminococcus fermentans |

3.1.2.a—Thiolester Hydrolase (CoA Specific)

In the CoA hydrolase family, the enzyme 3-hydroxyisobutyryl-CoA hydrolase is specific for 3-HIBCoA and has been described to efficiently catalyze the desired transformation during valine degradation (Shimomura et al. J Biol Chem 269:14248-14253 (1994)). Genes encoding this enzyme include hibch of Rattus norvegicus (Shimomura et al., supra (1994); Shimomura et al. Methods Enzymol. 324:229-240 (2000) and Homo sapiens (Shimomura et al., supra, 2000). Candidate genes by sequence homology include hibch of Saccharomyces cerevisiae and BC 2292 of Bacillus cereus.

| Gene | Accession No. | GI No. | Organism |
|---|---|---|---|
| hibch | Q5XIE6.2 | 146324906 | Rattus norvegicus |
| hibch | Q6NVY1.2 | 146324905 | Homo sapiens |
| hibch | P28817.2 | 2506374 | Saccharomyces cerevisiae |
| BC_2292 | Q81DR3 | 81434808 | Bacillus cereus |

The conversion of adipyl-CoA to adipate can be carried out by an acyl-CoA hydrolase or equivalently a thioesterase. The top E. coli gene candidate is tesB (Naggert et al. J Biol Chem. 266(17):11044-11050 (1991)] which shows high similarity to the human acot8 which is a dicarboxylic acid acetyltransferase with activity on adipyl-CoA (Westin et al. J Biol Chem 280(46): 38125-38132(2005). This activity has also been characterized in the rat liver (Deana, Biochem Int. 26(4): p. 767-773 (1992)).

| Gene | Accession No. | GI No. | Organism |
|---|---|---|---|
| tesB | NP_414986 | 16128437 | Escherichia coli |
| acot8 | CAA15502 | 3191970 | Homo sapiens |
| acot8 | NP_570112 | 51036669 | Rattus norvegicus |

Other potential E. coli thiolester hydrolases include the gene products of tesA (Bonner and Bloch, J Biol Chem. 247(10):3123-3133 (1972)), ybgC (Kuznetsova et al., FEMS Microbiol Rev. 29(2):263-279 (2005); Zhuang et al., FEBS Lett. 516(1-3):161-163 (2002)) paaI (Song et al., J Biol Chem. 281(16):11028-11038 (2006)), and ybdB (Leduc et al., J Bacteriol. 189(19):7112-7126 (2007)).

| Gene | Accession No. | GI No. | Organism |
|---|---|---|---|
| tesA | NP_415027 | 16128478 | Escherichia coli |
| ybgC | NP_415264 | 16128711 | Escherichia coli |
| paaI | NP_415914 | 16129357 | Escherichia coli |
| ybdB | NP_415129 | 16128580 | Escherichia coli |

Several eukaryotic acetyl-CoA hydrolases (EC 3.1.2.1) have broad substrate specificity. The enzyme from Rattus norvegicus brain (Robinson et al. Biochem. Biophys. Res. Commun. 71:959-965 (1976)) can react with butyryl-CoA, hexanoyl-CoA and malonyl-CoA.

| Gene | Accession No. | GI No. | Organism |
|---|---|---|---|
| acot12 | NP_570103.1 | 18543355 | Rattus norvegicus |

4.1.1.a—Carboxy-Lyase

An exemplary carboxy-lyase is acetolactate decarboxylase which participates in citrate catabolism and branched-chain amino acid biosynthesis, converting 2-acetolactate to acetoin. In Lactococcus lactis the enzyme is composed of six subunits, encoded by gene aldB, and is activated by valine, leucine and isoleucine (Goupil et al. Appl. Environ. Microbiol. 62:2636-2640 (1996); Goupil-Feuillerat et al. J. Bacteriol. 182:5399-5408 (2000)). This enzyme has been overexpressed and characterized in E. coli (Phalip et al. FEBS Lett. 351:95-99(1994)). In other organisms the enzyme is a dimer, encoded by aldC in Streptococcus thermophilus (Monnet et al. Lett. Appl. Microbiol. 36:399405 (2003)), aldB in Bacillus brevis (Diderichsen et al. J. Bacteriol. 172:4315-4321 (1990); Najmudin et al. Acta Crystallogr. D. Biol. Crystallogr. 59:1073-1075 (2003)) and budA from Enterobacter aerogenes (Diderichsen et al. J. Bacteriol. 172:4315-4321 (1990)). The enzyme from Bacillus brevis was cloned and overexpressed in Bacillus subtilis and characterized crystallographically (Najmudin et al. Acta Crystallogr. D. Biol. Crystallogr. 59:1073-1075 (2003)). Additionally, the enzyme from Leuconostoc lactis has been purified and characterized but the gene has not been isolated (O'Sullivan et al. FEMS Microbiol. Lett. 194:245-249 (2001)).

| Gene | Accession No. | GI No. | Organism |
|---|---|---|---|
| aldB | NP_267384.1 | 15673210 | Lactococcus lactis |
| aldC | Q8L208 | 75401480 | Streptococcus thermophilus |
| aldB | P23616.1 | 113592 | Bacillus brevis |
| budA | P05361.1 | 113593 | Enterobacter aerogenes |

Aconitate decarboxylase catalyzes the final step in itaconate biosynthesis in a strain of Candida and also in the filamentous fungus Aspergillus terreus (Bonnarme et al. J Bacteriol. 177:3573-3578 (1995); Willke and Vorlop Appl Microbiol Biotechnol 56:289-295 (2001)). Although itaconate is a compound of biotechnological interest, the aconitate decarboxylase gene or protein sequence has not been reported to date.

4-oxalocronate decarboxylase has been isolated from numerous organisms and characterized. Genes encoding this enzyme include dmpH and dmpE in Pseudomonas sp. (strain 600)(Shingler et al. J Bacteriol. 174:711-724 (1992)), xylII and xylIII from Pseudomonas putida (Kato and Asano Arch. Microbiol 168:457-463 (1997); Lian and Whitman J. Am. Chem. Soc. 116:10403-10411 (1994); Stanley et al. Biochemistry 39:3514 (2000)) and Reut_B5691 and Reut_B5692 from Ralstonia eutropha JMP134 (Hughes et al. J Bacteriol. 158:79-83 (1984)). The genes encoding the enzyme from Pseudomonas sp. (strain 600) have been cloned and expressed in E. coli (Shingler et al. J Bacteriol. 174:711-724 (1992)).

| Gene | Accession No. | GI No. | Organism |
|---|---|---|---|
| dmpH | CAA43228.1 | 45685 | Pseudomonas sp. CF600 |
| dmpE | CAA43225.1 | 45682 | Pseudomonas sp. CF600 |
| xylII | YP_709328.1 | 111116444 | Pseudomonas putida |
| xylIII | YP_709353.1 | 111116469 | Pseudomonas putida |
| Reut_B5691 | YP_299880.1 | 73539513 | Ralstonia eutropha JMP134 |
| Reut_B5692 | YP_299881.1 | 73539514 | Ralstonia eutropha JMP134 |

An additional class of decarboxylases has been characterized that catalyze the conversion of cinnamate (phenylacrylate) and substituted cinnamate derivatives to the corresponding styrene derivatives. These enzymes are common in a variety of organisms and specific genes encoding these enzymes that have been cloned and expressed in E. coli are: pad 1 from Saccharomyces cerevisae (Clausen et al. Gene 142:107-112(1994)), pdc from Lactobacillus plantarum (Barthelmebs et al. Appl Environ Microbiol 67:1063-1069 (2001); Qi et al. Metab Eng 9:268-276 (2007); Rodriguez et al. J. Agric. Food Chem. 56:3068-3072 (2008)), pofK (pad) from Klebsiella oxytoca (Hashidoko et al. Biosci. Biotech. Biochem. 58:217-218 (1994); Uchiyama et al. Biosci. Biotechnol. Biochem. 72:116-123 (2008)), Pedicoccus pentosaceus (Barthelmebs et al. Appl Environ Microbiol 67:1063-1069 (2001)), and padC from Bacillus subtilis and Bacillus pumilus (Lingen et al. Protein Eng 15:585-593 (2002)). A ferulic acid decarboxylase from Pseudomonas fluorescens also has been purified and characterized (Huang et al. J. Bacteriol. 176:5912-5918 (1994)). Importantly, this class of enzymes have been shown to be stable and do not require either exogenous or internally bound co-factors, thus making these enzymes ideally suitable for biotransformations (Sariaslani, Annu. Rev. Microbiol. 61:51-69 (2007)).

| Gene | Accession No. | GI No. | Organism |
|---|---|---|---|
| pad1 | AB368798 | 188496948 | Saccharomyces |
| | BAG32372.1 | 188496949 | cerevisae |
| pdc | U63827 | 1762615, 1762616 | Lactobacillus |
| | AAC45282.1 | | plantarum |
| pofK (pad) | AB330293, | 149941607, 149941608 | Klebsiella |
| | BAF65031.1 | | oxytoca |
| padC | AF017117 | 2394281, 2394282 | Bacillus |
| | AAC46254.1 | | subtilis |
| pad | AJ276891 | 11322456, 11322458 | Pedicoccus |
| | CAC16794.1 | | pentosaceus |
| pad | AJ278683 | 11691809, 11691810 | Bacillus |
| | CAC18719.1 | | pumilus |

Additional decarboxylase enzymes can form succinic semialdehyde from alpha-ketoglutarate. These include the alpha-ketoglutarate decarboxylase enzymes from Euglena gracilis (Shigeoka et al. Biochem. J. 282(Pt2):319-323 (1992); Shigeoka and Nakano Arch. Biochem. Biophys. 288:22-28 (1991); Shigeoka and Nakano Biochem. J. 292 (Pt 2):463-467 (1993)), whose corresponding gene sequence has yet to be determined, and from Mycobacterium tuberculosis (Tian et al. Proc Natl Acad Sci U.S.A. 102:10670-10675 (2005)). In addition, glutamate decarboxylase enzymes can convert glutamate into 4-aminobutyrate such as the products of the E. coli gadA and gadB genes (De Biase et al. Protein. Expr. Purif. 8:430-438 (1993)).

| Gene | Accession No. | GI No. | Organism |
|---|---|---|---|
| kgd | O50463.4 | 160395583 | Mycobacterium tuberculosis |
| gadA | NP_417974 | 16131389 | Escherichia coli |
| gadB | NP_416010 | 16129452 | Escherichia coli |

### Keto-Acid Decarboxylases

Pyruvate decarboxylase (PDC, EC 4.1.1.1), also termed keto-acid decarboxylase, is a key enzyme in alcoholic fermentation, catalyzing the decarboxylation of pyruvate to acetaldehyde. This enzyme has a broad substrate range for aliphatic 2-keto acids including 2-ketobutyrate, 2-ketovalerate, 3-hydroxypyruvate and 2-phenylpyruvate (Berg et al. Science 318:1782-1786 (2007)). The PDC from Zymomonas mobilus, encoded by pdc, has been a subject of directed engineering studies that altered the affinity for different substrates (Siegert et al. Protein Eng Des Sel 18:345-357 (2005)). The PDC from Saccharomyces cerevisiae has also been extensively studied, engineered for altered activity, and functionally expressed in E. coli(Killenberg-Jabs et al. Eur. J. Biochem. 268:1698-1704 (2001); Li and Jordan Biochemistry 38:10004-10012 (1999); ter Schure et al. Appl. Environ. Microbiol. 64:1303-1307 (1998)). The crystal structure of this enzyme is available (Killenberg-Jabs Eur. J. Biochem. 268:1698-1704 (2001)). Other well-characterized PDC candidates include the enzymes from Acetobacter pasteurians (Chandra et al. Arch. Microbiol. 176:443-451 (2001)) and Kluyveromyces lactis (Krieger et al. Eur. J. Biochem. 269:3256-3263 (2002)).

| Gene | Accession No. | GI No. | Organism |
|---|---|---|---|
| pdc | P06672.1 | 118391 | Zymomonas mobilus |
| pdc1 | P06169 | 30923172 | Saccharomyces cerevisiae |
| pdc | Q8L388 | 75401616 | Acetobacter pasteurians |
| pdc1 | Q12629 | 52788279 | Kluyveromyces lactis |

Like PDC, benzoylformate decarboxylase (EC 4.1.1.7) has a broad substrate range and has been the target of enzyme engineering studies. The enzyme from Pseudomonas putida has been extensively studied and crystal structures of this enzyme are available (Hasson et al. Biochemistry 37:9918-9930 (1998); Polovnikova et al. Biochemistry 42:1820-1830 (2003)). Site-directed mutagenesis of two residues in the active site of the Pseudomonas putida enzyme altered the affinity (Km) of naturally and non-naturally occurring substrates (Siegert Protein Eng Des Sel 18:345-357 (2005)). The properties of this enzyme have been further modified by directed engineering (Lingen et al. Protein Eng 15:585-593 (2002)); Lingen Chembiochem 4:721-726(2003)). The enzyme from Pseudomonas aeruginosa, encoded by mdlC, has also been characterized experimentally (Barrowman et al. FEMS Microbiology Letters 34:57-60 (1986)). Additional gene candidates from Pseudomonas stutzeri, Pseudomonas fluorescens and other organisms can be inferred by sequence homology or identified using a growth selection system developed in Pseudomonas putida (Henning et al. Appl. Environ. Microbiol. 72:7510-7517_(2006)).

| Gene | Accession No. | GI No. | Organism |
|---|---|---|---|
| mdlC | P20906.2 | 3915757 | Pseudomonas putida |
| mdlC | Q9HUR2.1 | 81539678 | Pseudomonas aeruginosa |
| dpgB | ABN80423.1 | 126202187 | Pseudomonas stutzeri |
| ilvB-1 | YP_260581.1 | 70730840 | Pseudomonas fluorescens |

4.2.1.a—Hydro-Lyase

The 2-(hydroxymethyl)glutarate dehydratase of Eubacterium barkeri is an exemplary hydro-lyase. This enzyme has been studied in the context of nicotinate catabolism and is encoded by hmd (Alhapel et al. Proc Natl Acad Sci USA 103:12341-12346 (2006)). Similar enzymes with high sequence homology are found in Bacteroides capillosus, Anaerotruncus colihominis, and Natranaerobius thermophilius.

| Gene | Accession No. | GI No. | Organism |
|---|---|---|---|
| hmd | ABC88407.1 | 86278275 | Eubacterium |
| | | | barkeri |
| BACCAP_02294 | ZP_02036683.1 | 154498305 | Bacteroides |
| | | | capillosus |
| | | | ATCC 29799 |
| ANACOL_02527 | ZP_02443222.1 | 167771169 | Anaerotruncus |
| | | | colihominis |
| | | | DSM 17241 |
| NtherDRAFT_2368 | ZP_02852366.1 | 169192667 | Natranaerobius |
| | | | thermophilus |
| | | | JW/NM-WN-LF |

A second exemplary hydro-lyase is fumarate hydratase, an enzyme catalyzing the dehydration of malate to fumarate. A wealth of structural information is available for this enzyme and researchers have successfully engineered the enzyme to alter activity, inhibition and localization (Weaver, T. Acta Crystallogr. D Biol Crystallogr. 61:1395-1401(2005)). Additional fumarate hydratases include those encoded by fumC from Escherichia coli (Estevez et al. Protein Sci. 11:1552-1557 (2002); Hong and Lee Biotechnol. Bioprocess Eng. 9:252-255 (2004); Rose and Weaver Proc Natl Acad Sci USA 101:3393-3397 (2004)), Campylobacter jejuni (Smith et al. Int. J Biochem. Cell Biol 31:961-975 (1999)) and Thermus thermophilus (Mizobata et al. Arch. Biochem. Biophys. 355:49-55 (1998)), and fumH from Rattus norvegicus (Kobayashi et al. J Biochem. 89:1923-1931(1981)). Similar enzymes with high sequence homology include fum1 from Arabidopsis thaliana and fumC from Corynebacterium glutamicum.

| Gene | Accession No. | GI No. | Organism |
|---|---|---|---|
| fumC | P05042.1 | 120601 | Escherichia coli K12 |
| fumC | O69294.1 | 9789756 | Campylobacter jejuni |
| fumC | P84127 | 75427690 | Thermus thermophilus |
| fumH | P14408.1 | 120605 | Rattus norvegicus |
| fum1 | P93033.2 | 39931311 | Arabidopsis thaliana |
| fumC | Q8NRN8.1 | 39931596 | Corynebacterium glutamicum |

Citramalate hydrolyase, also called 2-methylmalate dehydratase, converts 2-methylmalate to mesaconate. 2-Methylmalate dehydratase activity was detected in Clostridium tetanomorphum, Morganella morganii, Citrobacter amalonaticus in the context of the glutamate degradation VI pathway (Kato and Asano Arch. Microbiol 168:457463 (1997)); however the genes encoding this enzyme have not been sequenced to date.

The gene product of crt from C. acetobutylicum catalyzes the dehydration of 3-hydroxybutyryl-CoA to crotonyl-CoA (Atsumi et al. Metab Eng.; 29 (2007)); Boynton et al. Journal of Bacteriology 178:3015-3024(1996)). The enoyl-CoA hydratases, phaA and phaB, of P. putida are believed to carry out the hydroxylation of double bonds during phenylacetate catabolism; (Olivera et al. Proc Natl Acad Sci USA 95(11):6419-6424 (1998)). The paaA and paaB from P. fluorescens catalyze analogous transformations (14 Olivera et al., supra, 1998). Lastly, a number of Escherichia coli genes have been shown to demonstrate enoyl-CoA hydratase functionality including maoC (Park and Lee J Bacteriol 185(18):5391-5397 (2003)), paaF (Park and Lee Biotechnol Bioeng. 86(6):681-686 (2004a)); Park and Lee Appl Biochem Biotechnol. 113-116: 335-346 (2004b)); Ismail et al. Eur J Biochem 270(14):p. 3047-3054 (2003), and paaG (Park and Lee, supra, 2004; Park and Lee supra, 2004b; Ismail et al., supra, 2003).

| Gene | Accession No. | GI No. | Organism |
|---|---|---|---|
| maoC | NP_415905.1 | 16129348 | Escherichia coli |
| paaF | NP_415911.1 | 16129354 | Escherichia coli |
| paaG | NP_415912.1 | 16129355 | Escherichia coli |
| crt | NP_349318.1 | 15895969 | Clostridium acetobutylicum |
| paaA | NP_745427.1 | 26990002 | Pseudomonas putida |
| paaB | NP_745426.1 | 26990001 | Pseudomonas putida |
| phaA | ABF82233.1 | 106636093 | Pseudomonas fluorescens |
| phaB | ABF82234.1 | 106636094 | Pseudomonas fluorescens |

The E. coli genes fadA and fadB encode a multienzyme complex that exhibits ketoacyl-CoA thiolase, 3-hydroxyacyl-CoA dehydrogenase, and enoyl-CoA hydratase activities (Yang et al. Biochemistry 30(27): p. 6788-6795 (1991); Yang et al. J Biol Chem 265(18): p. 10424-10429 (1990); Yang et al. J Biol Chem 266(24): p. 16255 (1991); Nakahigashi and Inokuchi Nucleic Acids Res 18(16): p. 4937 (1990)). The fadI and fadJ genes encode similar functions and are naturally expressed only anaerobically (Campbell et al. Mol Microbiol 47(3): p. 793-805 (2003). A method for producing poly[(R)-3-hydroxybutyrate] in E. coli that involves activating fadB (by knocking out a negative regulator, fadR) and co-expressing a non-native ketothiolase (phaA from Ralstonia eutropha) has been described previously (Sato et al. J Biosci Bioeng 103(1): 38-44(2007)). This work clearly demonstrates that a β-oxidation enzyme, in particular the gene product of fadB which encodes both 3-hydroxyacyl-CoA dehydrogenase and enoyl-CoA hydratase activities, can function as part of a pathway to produce longer chain molecules from acetyl-CoA precursors.

| Gene | Accession No. | GI No. | Organism |
|---|---|---|---|
| fadA | YP_026272.1 | 49176430 | Escherichia coli |
| fadB | NP_418288.1 | 16131692 | Escherichia coli |
| fadI | NP_416844.1 | 16130275 | Escherichia coli |
| fadJ | NP_416843.1 | 16130274 | Escherichia coli |
| fadR | NP_415705.1 | 16129150 | Escherichia coli |

4.3.1.a—Ammonia-Lyase

Aspartase (EC 4.3.1.1), catalyzing the deamination of aspartate to fumarate, is a widespread enzyme in microorganisms, and has been characterized extensively (Viola, R. E. Adv. Enzymol. Relat Areas Mol. Biol 74:295-341 (2000)). The crystal structure of the E col aspartase, encoded by aspA, has been solved (Shi et al. Biochemistry 36:9136-9144 (1997)). The E col enzyme has also been shown to react with alternate substrates aspartatephenylmethylester, asparagine, benzyl-aspartate and malate (Ma et al. Ann N.Y. Acad Sci 672:60-65 (1992)). In a separate study, directed evolution was been employed on this enzyme to alter substrate specificity (Asano et al. Biomol. Eng 22:95-101 (2005)). Enzymes with aspartase functionality have also been characterized in Haemophilus influenzae (Sjostrom et al. Biochim. Biophys. Acta 1324:182-190 (1997)), Pseudomonas fluorescens (Takagi et al. J. Biochem. 96:545-552 (1984)), Bacillus subtilus (Sjostrom et al. Biochim. Biophys. Acta 1324:182-190 (1997)) and Serratia marcescens (Takagi and Kisumi J Bacteriol. 161:1-6 (1985)).

| Gene | Accession No. | GI No. | Organism |
|---|---|---|---|
| aspA | NP_418562 | 90111690 | Escherichia coli |
| | | | K12 subsp. MG 1655 |
| aspA | P44324.1 | 1168534 | Haemophilus influenzae |
| aspA | P07346.1 | 114273 | Pseudomonas fluorescens |
| ansB | P26899.1 | 114271 | Bacillus subtilus |
| aspA | P33109.1 | 416661 | Serratia marcescens |

3-methylaspartase (EC 4.3.1.2), also known as beta-methylaspartase or 3-methylaspartate ammonia-lyase, catalyzes the deamination of threo-3-methylasparatate to mesaconate. The 3-methylaspartase from Clostridium tetanomorphum has been cloned, functionally expressed in E. coli, and crystallized (Asuncion et al. Acta Crystallogr. D Biol Crystallogr. 57:731-733 (2001); Asuncion et al. J Biol Chem. 277:8306-8311 (2002); Botting et al. Biochemistry 27:2953-2955 (1988); Goda et al. Biochemistry 31:10747-10756(1992). In Citrobacter amalonaticus, this enzyme is encoded by BAA28709 (Kato and Asano Arch. Microbiol 168:457463 (1997)). 3-Methylaspartase has also been crystallized from E. coli YG1002 (Asano and Kato FEMS Microbiol Lett. 118:255-258 (1994)) although the protein sequence is not listed in public databases such as GenBank. Sequence homology can be used to identify additional candidate genes, including CTC_02563 in C. tetani and ECs0761 in Escherichia coli O157:H7.

| Gene | Accession No. | GI No. | Organism |
|---|---|---|---|
| MAL | AAB24070.1 | 259429 | Clostridium tetanomorphum |
| BAA28709 | BAA28709.1 | 3184397 | Citrobacter amalonaticus |
| CTC_02563 | NP_783085.1 | 28212141 | Clostridium tetani |
| ECs0761 | BAB34184.1 | 13360220 | Escherichia coli |
| | | | O157:H7 str. Sakai |

Ammonia-lyase enzyme candidates that form enoyl-CoA products include beta-alanyl-CoA ammonia-lyase (EC 4.3.1.6), which deaminates beta-alanyl-CoA, and 3-aminobutyryl-CoA ammonia-lyase (EC 4.3.1.14). Two beta-alanyl-CoA ammonia lyases have been identified and characterized in Clostridium propionicum (Herrmann et al. FEBS J. 272:813-821 (2005)). No other beta-alanyl-CoA ammonia lyases have been studied to date, but gene candidates can be identified by sequence similarity. One such candidate is MXAN_4385 in Myxococcus xanthus.

| Gene | Accession No. | GI No. | Organism |
|---|---|---|---|
| acl2 | CAG29275.1 | 47496504 | Clostridium propionicum |
| acl1 | CAG29274.1 | 47496502 | Clostridium propionicum |
| MXAN_4385 | YP_632558.1 | 108756898 | Myxococcus xanthus |

5.3.3.a—Isomerase

The 4-hydroxybutyryl-CoA dehydratases from both Clostridium aminobutyricum and C. kluyveri catalyze the reversible conversion of 4-hydroxybutyryl-CoA to crotonyl-CoA and posses an intrinsic vinylacetyl-CoA Δ-isomerase activity (Scherf and Buckel Eur. J Biochem. 215:421-429 (1993); Scherf et al. Arch. Microbiol 161:239-245 (1994)). Both native enzymes were purified and characterized, including the N-terminal amino acid sequences (Scherf and Buckel, supra, 1993; Scherf et al., supra, 1994). The abfD genes from C. aminobutyricum and C. kluyveri match exactly with these N-terminal amino acid sequences, thus are encoding the 4-hydroxybutyryl-CoA dehydratases/vinylacetyl-CoA Δ-isomerase. In addition, the abfD gene from Porphyromonas gingivalis ATCC 33277 is identified through homology from genome projects.

| Gene | Accession No. | GI No. | Organism |
|---|---|---|---|
| abfD | YP_001396399.1 | 153955634 | Clostridium kluyveri DSM 555 |
| abfD | P55792 | 84028213 | Clostridium aminobutyricum |
| abfD | YP_001928843 | 188994591 | Porphyromonas gingivalis |
| | | | ATCC 33277 |

5.4.3.a—Aminomutase

Lysine 2,3-aminomutase (EC 5.4.3.2) is an exemplary aminomutase that converts lysine to (3S)-3,6-diaminohexanoate, shifting an amine group from the 2- to the 3-position. The enzyme is found in bacteria that ferment lysine to acetate and butyrate, including as Fusobacterium nuleatum (kanA)(Barker et al. J. Bacteriol. 152:201-207 (1982)) and Clostridium subterminale (kamA)(Chirpich et al. J. Biol. Chem. 245:1778-1789 (1970)). The enzyme from Clostridium subterminale has been crystallized (Lepore et al. Proc. Natl. Acad. Sci. U.S.A 102:13819-13824 (2005)). An enzyme encoding this function is also encoded by yodO in Bacillus subtilus (Chen et al. Biochem. J. 348 Pt 3:539-549 (2000)). The enzyme utilizes pyridoxal 5′-phosphate as a cofactor, requires activation by S-Adenosylmethionine, and is stereoselective, reacting with the only with L-lysine. The enzyme has not been shown to react with alternate substrates.

| Gene | Accession No. | GI No. | Organism |
|---|---|---|---|
| yodO | O34676.1 | 4033499 | Bacillus subtilus |
| kamA | Q9XBQ8.1 | 75423266 | Clostridium subterminale |
| kamA | Q8RHX4 | 81485301 | Fusobacterium nuleatum |
| | | | subsp. nuleatum |

A second aminomutase, beta-lysine 5,6-aminomutase (EC 5.4.3.3), catalyzes the next step of lysine fermentation to acetate and butyrate, which transforms (3S)-3,6-diaminohexanoate to (3S,5S)-3,5-diaminohexanoate, shifting a terminal amine group from the 6- to the 5-position. This enzyme also catalyzes the conversion of lysine to 2,5-diaminohexanoate and is also called lysine-5,6-aminomutase (EC 5.4.3.4). The enzyme has been crystallized in Clostridium sticklandii(kamD, kamE)(Berkovitch et al. Proc. Natl. Acad. Sci. USA 101:15870-15875 (2004)). The enzyme from Porphyromonas gingivalis has also been characterized (Tang et al. Biochemistry 41:8767-8776 (2002)).

| Gene | Accession No. | GI No. | Organism |
|---|---|---|---|
| kamD | AAC79717.1 | 3928904 | Clostridium sticklandii |
| kamE | AAC79718.1 | 3928905 | Clostridium sticklandii |
| kamD | NC_002950.2 | 34539880, | Porphyromonas gingivalis |
| | | 34540809 | W83 |
| kamE | NC_002950.2 | 34539880, | Porphyromonas gingivalis |
| | | 34540810 | W83 |

Omithine 4,5-aminomutase (EC 5.4.3.5) converts D-omithine to 2,4-diaminopentanoate, also shifting a terminal amine to the adjacent carbon. The enzyme from Clostridium sticklandi is encoded by two genes, oraE and oraS, and has been cloned, sequenced and expressed in E. coli (Chen et al. J. Biol. Chem. 276:44744-44750 (2001)). This enzyme has not been characterized in other organisms to date.

| Gene | Accession No. | GI No. | Organism |
|---|---|---|---|
| oraE | AAK72502 | 17223685 | Clostridium sticklandii |
| oraS | AAK72501 | 17223684 | Clostridium sticklandii |

Tyrosine 2,3-aminomutase (EC 5.4.3.6) participates in tyrosine biosynthesis, reversibly converting tyrosine to 3-amino-3-(4-hydroxyphenyl)propanoate by shifting an amine from the 2- to the 3-position. In Streptomyces globisporus the enzyme has also been shown to react with tyrosine derivatives (Christenson et al. Biochemistry 42:12708-12718 (2003)). Sequence information is not available.

Leucine 2,3-aminomutase (EC 5.4.3.7) converts L-leucine to beta-leucine during leucine degradation and biosynthesis. An assay for leucine 2,3-aminomutase detected activity in many organisms (Poston, J. M. Methods Enzymol. 166:130-135 (1988)) but genes encoding the enzyme have not been identified to date.

Cargill has developed a novel 2,3-aminomutase enzyme to convert L-alanine to β-alanine, thus creating a pathway from pyruvate to 3-HP in four biochemical steps (Liao et al., U.S. Publication No. 2005-0221466).

6.2.1.a—Acid-Thiol Ligase

An exemplary acid-thiol ligase is the gene products of sucCD of E. coli which together catalyze the formation of succinyl-CoA from succinate with the contaminant consumption of one ATP, a reaction which is reversible in vivo (Buck et al. Biochemistry 24(22): p. 6245-6252 (1985)). Additional exemplary CoA-ligases include the rat dicarboxylate-CoA ligase for which the sequence is yet uncharacterized (Vamecq et al. Biochem J. 230(3): p. 683-693 (1985)), either of the two characterized phenylacetate-CoA ligases from P. chrysogenum (Lamas-Maceiras et al. Biochem J 395(1):147-155 (2006); Wang et al. Biochem Biophys Res Commun, 360(2):453-458 (2007)), the phenylacetate-CoA ligase from Pseudomonas putida (Martinez-Blanco et al. J Biol Chem. 265(12):7084-7090 (1990)), and the 6-carboxyhexanoate-CoA ligase from Bacillus subtilis (Bower et al. J Bacteriol 178(14):4122-4130 (1996)).

| Gene | Accession No. | GI No. | Organism |
|---|---|---|---|
| sucC | NP_415256.1 | 16128703 | Escherichia coli |
| sucD | AAC73823.1 | 1786949 | Escherichia coli |
| phl | CAJ15517.1 | 77019264 | Penicillium chrysogenum |
| phlB | ABS19624.1 | 152002983 | Penicillium chrysogenum |
| paaF | AAC24333.2 | 22711873 | Pseudomonas putida |
| bioW | NP_390902.2 | 50812281 | Bacillus subtilis |

### Example V

### Exemplary BDO Pathway from Succinyl-CoA

This example describes exemplary BDO pathways from succinyl-CoA.

BDO pathways from succinyl-CoA are described herein and have been described previously (see U.S. application Ser. No. 12/049,256, filed Mar. 14, 2008, and PCT application serial No. US08/57168, filed Mar. 14, 2008, each of which is incorporated herein by reference). Additional pathways are shown in FIG. 8A. Enzymes of such exemplary BDO pathways are listed in Table 15, along with exemplary genes encoding these enzymes.

Briefly, succinyl-CoA can be converted to succinic semialdehyde by succinyl-CoA reductase (or succinate semialdehyde dehydrogenase)(EC 1.2.1.b). Succinate semialdehyde can be converted to 4-hydroxybutyrate by 4-hydroxybutyrate dehydrogenase (EC 1.1.1.a), as previously described. Alternatively, succinyl-CoA can be converted to 4-hydroxybutyrate by succinyl-CoA reductase (alcohol forming)(EC 1.1.1.c). 4-Hydroxybutyrate can be converted to 4-hydroxybutyryl-CoA by 4-hydroxybutyryl-CoA transferase (EC 2.8.3.a), as previously described, or by 4-hydroxybutyryl-CoA hydrolase (EC 3.1.2.a) or 4-hydroxybutyryl-CoA ligase (or 4-hydroxybutyryl-CoA synthetase)(EC 6.2.1.a). Alternatively, 4-hydroxybutyrate can be converted to 4-hydroxybutyryl-phosphate by 4-hydroxybutyrate kinase (EC 2.7.2.a), as previously described. 4-Hydroxybutyryl-phosphate can be converted to 4-hydroxybutyryl-CoA by phosphotrans-4-hydroxybutyrylase (EC 2.3.1.a), as previously described. Alternatively, 4-hydroxybutyryl-phosphate can be converted to 4-hydroxybutanal by 4-hydroxybutanal dehydrogenase (phosphorylating)(EC 1.2.1.d). 4-Hydroxybutyryl-CoA can be converted to 4-hydroxybutanal by 4-hydroxybutyryl-CoA reductase (or 4-hydroxybutanal dehydrogenase)(EC 1.2.1.b). Alternatively, 4-hydroxybutyryl-CoA can be converted to 1,4-butanediol by 4-hydroxybutyryl-CoA reductase (alcohol forming)(EC 1.1.1.c). 4-Hydroxybutanal can be converted to 1,4-butanediol by 1,4-butanediol dehydrogenase (EC 1.1.1.a), as previously described.

TABLE 15
BDO pathway from succinyl-CoA.

| | EC | Desired | Desired | | Gene | GenBank ID | | Known |
|---|---|---|---|---|---|---|---|---|
| FIG. | class | substrate | product | Enzyme name | name | (if available) | Organism | Substrates |
| 8A | 1.2.1.b | succinyl-CoA | succinic | succinyl-CoA | sucD | P38947.1 | Clostridium | succinyl-CoA |
| | | | semialdehyde | reductase | | | kluyveri | |
| | | | | (or succinate | | | | |
| | | | | semialdehyde | | | | |
| | | | | dehydrogenase) | | | | |
| | | | | | sucD | NP_904963.1 | Porphyromonas | succinyl-CoA |
| | | | | | | | gingivalis | |
| | | | | | Msed_0709 | YP_001190808.1 | Metallosphaera | malonyl-CoA |
| | | | | | | | sedula | |
| 8A | 1.1.1.a | succinate | 4-hydroxy- | 4-hydroxybutyrate | 4hbd | YP_726053.1 | Ralstonia | 4-hydroxy- |
| | | semialdehyde | butyrate | dehydrogenase | | | eutropha H16 | butyrate |
| | | | | | 4hbd | L21902.1 | Clostridium | 4-hydroxy- |
| | | | | | | | kluyveri DSM 555 | butyrate |
| | | | | | 4hbd | Q94B07 | Arabidopsis | 4-hydroxy- |
| | | | | | | | thaliana | butyrate |
| 8A | 1.1.1.c | succinyl-CoA | 4-hydroxy- | succinyl-CoA | adhE2 | AAK09379.1 | Clostridium | butanoyl-CoA |
| | | | butyrate | reductase | | | acetobutylicum | |
| | | | | (alcohol forming) | | | | |
| | | | | | mcr | AAS20429.1 | Chloroflexus | malonyl-CoA |
| | | | | | | | aurantiacus | |
| | | | | | FAR | AAD38039.1 | Simmondsia | long chain |
| | | | | | | | chinensis | acyl-CoA |
| 8A | 2.8.3.a | 4-hydroxy- | 4-hydroxy- | 4-hydroxy- | cat1, cat2, | P38946.1, | Clostridium | succinate, 4- |
| | | butyrate | butyryl-CoA | butyryl-CoA | cat3 | P38942.2, | kluyveri | hydroxybutyrate, |
| | | | | transferase | | EDK35586.1 | | butyrate |
| | | | | | gctA, gctB | CAA57199.1, | Acidaminococcus | glutarate |
| | | | | | | CAA57200.1 | fermentans | |
| | | | | | atoA, atoD | P76459.1, | Escherichia | butanoate |
| | | | | | | P76458.1 | coli | |
| 8A | 3.1.2.a | 4-hydroxy- | 4-hydroxy- | 4-hydroxy- | tesB | NP_414986 | Escherichia | adipyl-CoA |
| | | butyrate | butyryl-CoA | butyryl-CoA | | | coli | |
| | | | | hydrolase | | | | |
| | | | | | acot12 | NP_570103.1 | Rattus | butyryl-CoA |
| | | | | | | | norvegicus | |
| | | | | | hibch | Q6NVY1.2 | Homo sapiens | 3-hydroxy- |
| | | | | | | | | propanoyl-CoA |
| 8A | 6.2.1.a | 4-hydroxy- | 4-hydroxy- | 4-hydroxy- | sucCD | NP_415256.1, | Escherichia | succinate |
| | | butyrate | butyryl-CoA | butyryl-CoA | | AAC73823.1 | coli | |
| | | | | ligase (or 4- | | | | |
| | | | | hydroxybutyryl- | | | | |
| | | | | CoA synthetase) | | | | |
| | | | | | phl | CAJ15517.1 | Penicillium | phenylacetate |
| | | | | | | | chrysogenum | |
| | | | | | bioW | NP_390902.2 | Bacillus | 6-carboxy- |
| | | | | | | | subtilis | hexanoate |
| 8A | 2.7.2.a | 4-hydroxy- | 4-hydroxy- | 4-hydroxy- | ackA | NP_416799.1 | Escherichia | acetate, |
| | | butyrate | butyryl- | butyrate | | | coli | propionate |
| | | | phosphate | kinase | | | | |
| | | | | | buk1 | NP_349675 | Clostridium | butyrate |
| | | | | | | | acetobutylicum | |
| | | | | | buk2 | Q97II1 | Clostridium | butyrate |
| | | | | | | | acetobutylicum | |
| 8A | 2.3.1.a | 4-hydroxy- | 4-hydroxy- | phosphotrans-4- | ptb | NP_349676 | Clostridium | butyryl- |
| | | butyryl- | butyryl-CoA | hydroxybutyrylase | | | acetobutylicum | phosphate |
| | | phosphate | | | | | | |
| | | | | | ptb | AAR19757.1 | butyrate- | butyryl- |
| | | | | | | | producing | phosphate |
| | | | | | | | bacterium L2-50 | |
| | | | | | ptb | CAC07932.1 | Bacillus | butyryl- |
| | | | | | | | megaterium | phosphate |
| 8A | 1.2.1.d | 4-hydroxy- | 4-hydroxy- | 4-hydroxybutanal | asd | NP_417891.1 | Escherichia | L-4-aspartyl- |
| | | butyryl- | butanal | dehydrogenase | | | coli | phosphate |
| | | phosphate | | (phosphorylating) | | | | |
| | | | | | proA | NP_414778.1 | Escherichia | L-glutamyl- |
| | | | | | | | coli | 5-phospate |
| | | | | | gapA | P0A9B2.2 | Escherichia | Glyceraldehyde- |
| | | | | | | | coli | 3-phosphate |
| 8A | 1.2.1.b | 4-hydroxy- | 4-hydroxy- | 4-hydroxy- | sucD | P38947.1 | Clostridium | succinyl-CoA |
| | | butyryl-CoA | butanal | butyryl-CoA | | | kluyveri | |
| | | | | reductase (or 4- | | | | |
| | | | | hydroxybutanal | | | | |
| | | | | dehydrogenase) | | | | |
| | | | | | sucD | NP_904963.1 | Porphyromonas | succinyl-CoA |
| | | | | | | | gingivalis | |
| | | | | | Msed_0709 | YP_001190808.1 | Metallosphaera | malonyl-CoA |
| | | | | | | | sedula | |
| 8A | 1.1.1.c | 4-hydroxy- | 1,4- | 4-hydroxy- | adhE2 | AAK09379.1 | Clostridium | butanoyl-CoA |
| | | butyryl-CoA | butanediol | butyryl-CoA | | | acetobutylicum | |
| | | | | reductase | | | | |
| | | | | (alcohol forming) | | | | |
| | | | | | mcr | AAS20429.1 | Chloroflexus | malonyl-CoA |
| | | | | | | | aurantiacus | |
| | | | | | FAR | AAD38039.1 | Simmondsia | long chain |
| | | | | | | | chinensis | acyl-CoA |
| 8A | 1.1.1.a | 4-hydroxy- | 1,4- | 1,4-butanediol | ADH2 | NP_014032.1 | Saccharymyces | general |
| | | butanal | butanediol | dehydrogenase | | | cerevisiae | |
| | | | | | yqhD | NP_417484.1 | Escherichia | >C3 |
| | | | | | | | coli | |
| | | | | | 4hbd | L21902.1 | Clostridium | Succinate |
| | | | | | | | kluyveri DSM 555 | semialdehyde |

### Example VI

### Additional Exemplary BDO Pathways from Alpha-Ketoglutarate

This example describes exemplary BDO pathways from alpha-ketoglutarate.

BDO pathways from succinyl-CoA are described herein and have been described previously (see U.S. application Ser. No. 12/049,256, filed Mar. 14, 2008 (US publication 20090075351) and PCT application serial No. US08/57168, filed Mar. 14, 2008 (WO/2008/115840), each of which is incorporated herein by reference). Additional pathways are shown in FIG. 8B. Enzymes of such exemplary BDO pathways are listed in Table 16, along with exemplary genes encoding these enzymes.

Briefly, alpha-ketoglutarate can be converted to succinic semialdehyde by alpha-ketoglutarate decarboxylase (EC 4.1.1.a), as previously described. Alternatively, alpha-ketoglutarate can be converted to glutamate by glutamate dehydrogenase (EC 1.4.1.a). 4-Aminobutyrate can be converted to succinic semialdehyde by 4-aminobutyrate oxidoreductase (deaminating)(EC 1.4.1.a) or 4-aminobutyrate transaminase (EC 2.6.1.a). Glutamate can be converted to 4-aminobutyrate by glutamate decarboxylase (EC 4.1.1.a). Succinate semialdehyde can be converted to 4-hydroxybutyrate by 4-hydroxybutyrate dehydrogenase (EC 1.1.1.a), as previously described. 4-Hydroxybutyrate can be converted to 4-hydroxybutyryl-CoA by 4-hydroxybutyryl-CoA transferase (EC 2.8.3.a), as previously described, or by 4-hydroxybutyryl-CoA hydrolase (EC 3.1.2.a), or 4-hydroxybutyryl-CoA ligase (or 4-hydroxybutyryl-CoA synthetase) (EC 6.2.1.a). 4-Hydroxybutyrate can be converted to 4-hydroxybutyryl-phosphate by 4-hydroxybutyrate kinase (EC 2.7.2.a). 4-Hydroxybutyryl-phosphate can be converted to 4-hydroxybutyryl-CoA by phosphotrans-4-hydroxybutyrylase (EC 2.3.1.a), as previously described. Alternatively, 4-hydroxybutyryl-phosphate can be converted to 4-hydroxybutanal by 4-hydroxybutanal dehydrogenase (phosphorylating)(EC 1.2.1.d). 4-Hydroxybutyryl-CoA can be converted to 4-hydroxybutanal by 4-hydroxybutyryl-CoA reductase (or 4-hydroxybutanal dehydrogenase)(EC 1.2.1.b), as previously described. 4-Hydroxybutyryl-CoA can be converted to 1,4-butanediol by 4-hydroxybutyryl-CoA reductase (alcohol forming)(EC 1.1.1.c). 4-Hydroxybutanal can be converted to 1,4-butanediol by 1,4-butanediol dehydrogenase (EC 1.1.1.a), as previously described.

TABLE 16
BDO pathway from alpha-ketoglutarate.

| | EC | Desired | Desired | | Gene | GenBank ID | | Known |
|---|---|---|---|---|---|---|---|---|
| FIG. | class | substrate | product | Enzyme name | name | (if available) | Organism | Substrates |
| 8B | 4.1.1.a | alpha-keto- | succinic | alpha-keto- | kgd | O50463.4 | Mycobacterium | alpha-keto- |
| | | glutarate | semialdehyde | glutarate | | | tuberculosis | glutarate |
| | | | | decarboxylase | | | | |
| | | | | | gadA | NP_417974 | Escherichia | glutamate |
| | | | | | | | coli | |
| | | | | | gadB | NP_416010 | Escherichia | glutamate |
| | | | | | | | coli | |
| 8B | 1.4.1.a | alpha-keto- | glutamate | glutamate | gdhA | P00370 | Escherichia | glutamate |
| | | glutarate | | dehydrogenase | | | coli | |
| | | | | | gdh | P96110.4 | Thermotoga | glutamate |
| | | | | | | | maritima | |
| | | | | | gdhA1 | NP_279651.1 | Halobacterium | glutamate |
| | | | | | | | salinarum | |
| 8B | 1.4.1.a | 4-amino- | succinic | 4-aminobutyrate | lysDH | AB052732 | Geobacillus | lysine |
| | | butyrate | semialdehyde | oxidoreductase | | | stearothermophilus | |
| | | | | (deaminating) | | | | |
| | | | | | lysDH | NP_147035.1 | Aeropyrum | lysine |
| | | | | | | | pernix K1 | |
| | | | | | ldh | P0A393 | Bacillus | leucine, |
| | | | | | | | cereus | isoleucine, |
| | | | | | | | | valine, 2- |
| | | | | | | | | aminobutanoate |
| 8B | 2.6.1.a | 4-amino- | succinic | 4-aminobutyrate | gabT | P22256.1 | Escherichia | 4-amino- |
| | | butyrate | semialdehyde | transaminase | | | coli | butyryate |
| | | | | | puuE | NP_415818.1 | Escherichia | 4-amino- |
| | | | | | | | coli | butyryate |
| | | | | | UGA1 | NP_011533.1 | Saccharomyces | 4-amino- |
| | | | | | | | cerevisiae | butyryate |
| 8B | 4.1.1.a | glutamate | 4-amino- | glutamate | gadA | NP_417974 | Escherichia | glutamate |
| | | | butyrate | decarboxylase | | | coli | |
| | | | | | gadB | NP_416010 | Escherichia | glutamate |
| | | | | | | | coli | |
| | | | | | kgd | O50463.4 | Mycobacterium | alpha-keto- |
| | | | | | | | tuberculosis | glutarate |
| 8B | 1.1.1.a | succinate | 4-hydroxy- | 4-hydroxybutyrate | 4hbd | YP_726053.1 | Ralstonia | 4-hydroxy- |
| | | semialdehyde | butyrate | dehydrogenase | | | eutropha H16 | butyrate |
| | | | | | 4hbd | L21902.1 | Clostridium | 4-hydroxy- |
| | | | | | | | kluyveri DSM 555 | butyrate |
| | | | | | 4hbd | Q94B07 | Arabidopsis | 4-hydroxy- |
| | | | | | | | thaliana | butyrate |
| 8B | 2.8.3.a | 4-hydroxy- | 4-hydroxy- | 4-hydroxybutyryl- | cat1, cat2, | P38946.1, | Clostridium | succinate, 4- |
| | | butyrate | butyryl-CoA | CoA transferase | cat3 | P38942.2, | kluyveri | hydroxybutyrate, |
| | | | | | | EDK35586.1 | | butyrate |
| | | | | | gctA, gctB | CAA57199.1, | Acidaminococcus | glutarate |
| | | | | | | CAA57200.1 | fermentans | |
| | | | | | atoA, atoD | P76459.1, | Escherichia | butanoate |
| | | | | | | P76458.1 | coli | |
| 8B | 3.1.2.a | 4-hydroxy- | 4-hydroxy- | 4-hydroxybutyryl- | tesB | NP_414986 | Escherichia | adipyl-CoA |
| | | butyrate | butyryl-CoA | CoA hydrolase | | | coli | |
| | | | | | acot12 | NP_570103.1 | Rattus | butyryl-CoA |
| | | | | | | | norvegicus | |
| | | | | | hibch | Q6NVY1.2 | Homo sapiens | 3-hydroxy- |
| | | | | | | | | propanoyl-CoA |
| 8B | 6.2.1.a | 4-hydroxy- | 4-hydroxy- | 4-hydroxybutyryl- | sucCD | NP_415256.1, | Escherichia | succinate |
| | | butyrate | butyryl-CoA | CoA ligase (or 4- | | AAC73823.1 | coli | |
| | | | | hydroxybutyryl- | | | | |
| | | | | CoA synthetase) | | | | |
| | | | | | phl | CAJ15517.1 | Penicillium | phenylacetate |
| | | | | | | | chrysogenum | |
| | | | | | bioW | NP_390902.2 | Bacillus | 6-carboxy- |
| | | | | | | | subtilis | hexanoate |
| 8B | 2.7.2.a | 4-hydroxy- | 4-hydroxy- | 4-hydroxybutyrate | ackA | NP_416799.1 | Escherichia | acetate, |
| | | butyrate | butyryl- | kinase | | | coli | propionate |
| | | | phosphate | | | | | |
| | | | | | buk1 | NP_349675 | Clostridium | butyrate |
| | | | | | | | acetobutylicum | |
| | | | | | buk2 | Q97II1 | Clostridium | butyrate |
| | | | | | | | acetobutylicum | |
| 8B | 2.3.1.a | 4-hydroxy- | 4-hydroxy- | phosphotrans-4- | ptb | NP_349676 | Clostridium | butyryl- |
| | | butyryl- | butyryl-CoA | hydroxybutyrylase | | | acetobutylicum | phosphate |
| | | phosphate | | | | | | |
| | | | | | ptb | AAR19757.1 | butyrate- | butyryl- |
| | | | | | | | producing | phosphate |
| | | | | | | | bacterium L2-50 | |
| | | | | | ptb | CAC07932.1 | Bacillus | butyryl- |
| | | | | | | | megaterium | phosphate |
| 8B | 1.2.1.d | 4-hydroxy- | 4-hydroxy- | 4-hydroxybutanal | asd | NP_417891.1 | Escherichia | L-4-aspartyl- |
| | | butyryl- | butanal | dehydrogenase | | | coli | phosphate |
| | | phosphate | | (phosphorylating) | | | | |
| | | | | | proA | NP_414778.1 | Escherichia | L-glutamyl-5- |
| | | | | | | | coli | phospate |
| | | | | | gapA | P0A9B2.2 | Escherichia | Glyceraldehyde- |
| | | | | | | | coli | 3-phosphate |
| 8B | 1.2.1.b | 4-hydroxy- | 4-hydroxy- | 4-hydroxybutyryl- | sucD | P38947.1 | Clostridium | succinyl-CoA |
| | | butyryl-CoA | butanal | CoA reductase (or | | | kluyveri | |
| | | | | 4-hydroxybutanal | | | | |
| | | | | dehydrogenase) | | | | |
| | | | | | sucD | NP_904963.1 | Porphyromonas | succinyl-CoA |
| | | | | | | | gingivalis | |
| | | | | | Msed_0709 | YP_001190808.1 | Metallosphaera | malonyl-CoA |
| | | | | | | | sedula | |
| 8B | 1.1.1.c | 4-hydroxy- | 1,4- | 4-hydroxybutyryl- | adhE2 | AAK09379.1 | Clostridium | butanoyl-CoA |
| | | butyryl-CoA | butanediol | CoA reductase | | | acetobutylicum | |
| | | | | (alcohol forming) | | | | |
| | | | | | mcr | AAS20429.1 | Chloroflexus | malonyl-CoA |
| | | | | | | | aurantiacus | |
| | | | | | FAR | AAD38039.1 | Simmondsia | long chain |
| | | | | | | | chinensis | acyl-CoA |
| 8B | 1.1.1.a | 4-hydroxy- | 1,4- | 1,4-butanediol | ADH2 | NP_014032.1 | Saccharymyces | general |
| | | butanal | butanediol | dehydrogenase | | | cerevisiae | |
| | | | | | yqhD | NP_417484.1 | Escherichia | >C3 |
| | | | | | | | coli | |
| | | | | | 4hbd | L21902.1 | Clostridium | Succinate |
| | | | | | | | kluyveri DSM 555 | semialdehyde |

### Example VII

### BDO Pathways from 4-Aminobutyrate

This example describes exemplary BDO pathway from 4-aminobutyrate. See corresponding Example in WO2013/184602 and FIGS. 9A and 9B. FIG. 9C shows an exemplary pathway through acetoacetate.

### Example VIII

### Exemplary BDO Pathways from Alpha-Ketoglutarate

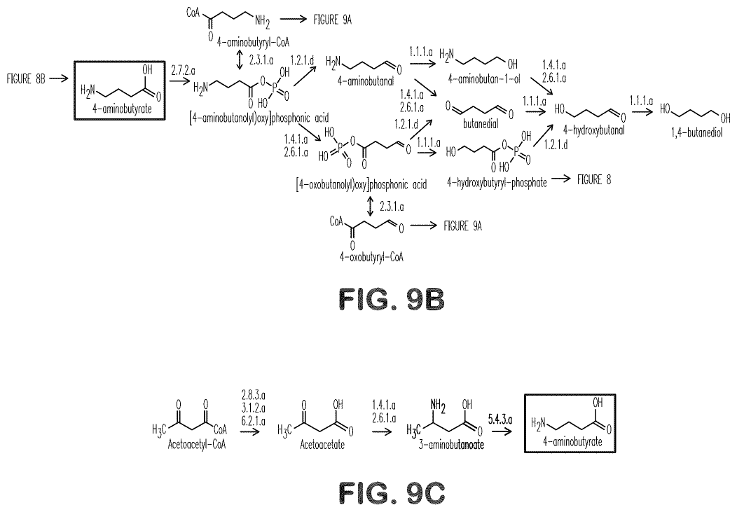

This example describes exemplary BDO pathways from alpha-ketoglutarate. See corresponding example in WO2013/186402 and FIG. 10.

### Example IX

### Exemplary BDO Pathways from Glutamate

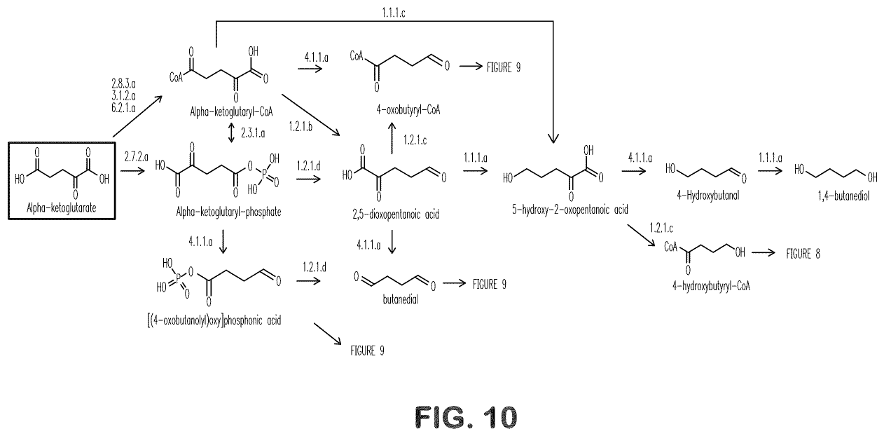

This example describes exemplary BDO pathways from glutamate. See corresponding example in WO2013/186402 and FIG. 11.

### Example X

### Exemplary BDO from Acetoacetyl-CoA

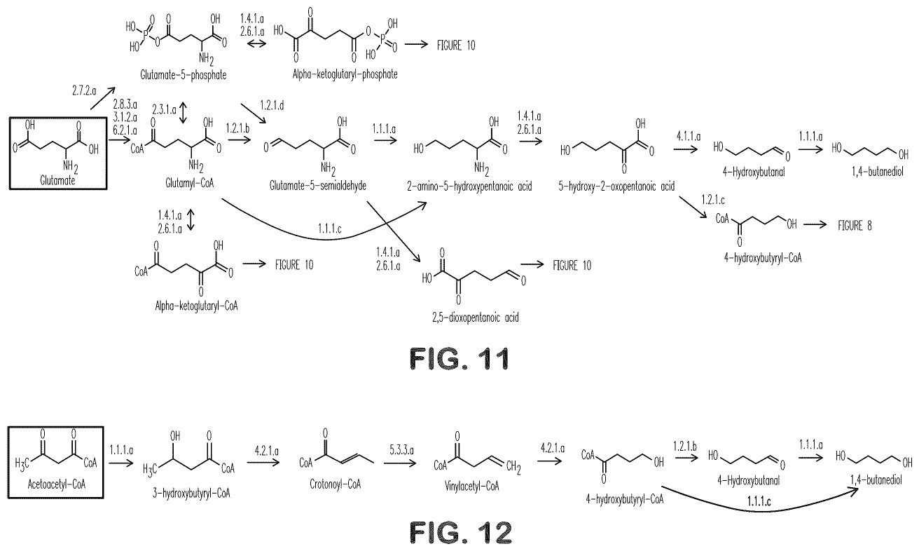

This example describes an exemplary BDO pathway from acetoacetyl-CoA. See corresponding example in WO2013/186402 and FIG. 12.

### Example XI

### Exemplary BDO Pathway from Homoserine

This example describes an exemplary BDO pathway from homoserine. See corresponding example in WO2013/186402 and FIG. 13.

### Example XII

### BDO Producing Strains Expressing Succinyl-CoA Synthetase

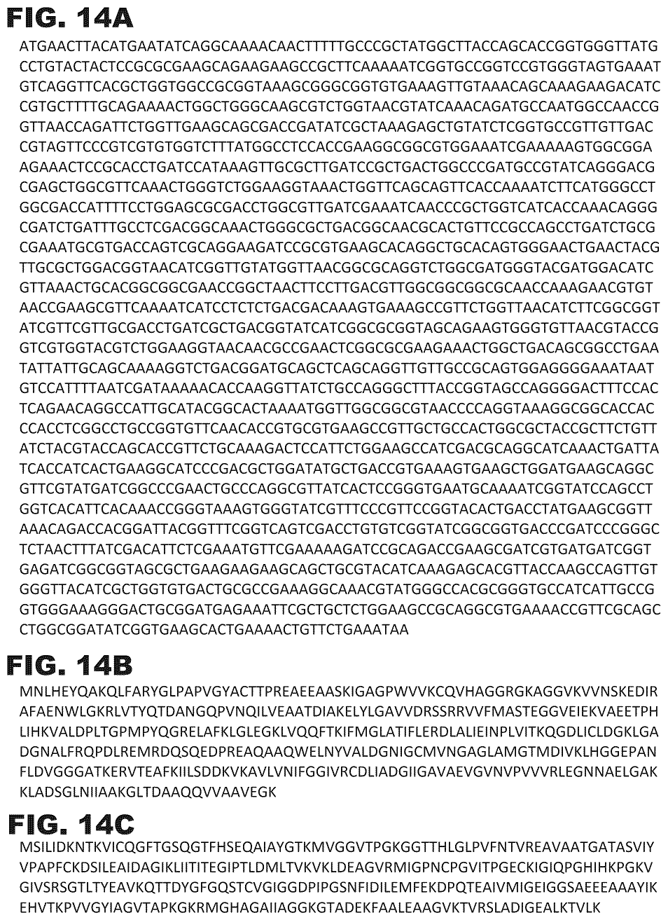

This example describes increased production of BDO in BDO producing strains expressing succinyl-CoA synthetase. See corresponding example in WO2013/186402. The nucleotide sequence of the E. coli sucCD operon is shown in FIG. 14A, and the amino acid sequences for the encoded succinyl-CoA synthetase subunits are shown in FIGS. 14B and 14C.

The E. coli sucCD genes, which encode the succinyl-CoA synthetase, were overexpressed. The results showed that introducing into the strains sucCD to express succinyl-CoA synthetase improved BDO production in various strains compared to either native levels of expression or expression of cat1, which is a succinyl-CoA/acetyl-CoA transferase. Thus, BDO production was improved by overexpressing the native E. coli sucCD genes encoding succinyl-CoA synthetase.

### Example XIII

### Expression of Heterologous Genes Encoding BDO Pathway Enzymes

This example describes the expression of various non-native pathway enzymes to provide improved production of BDO. See corresponding Example in WO2013/184602.

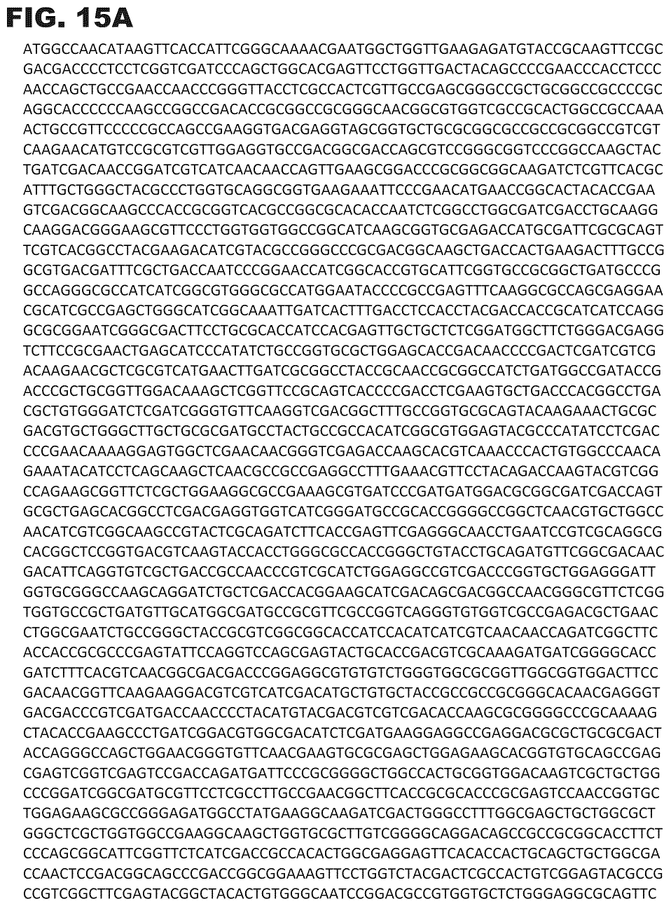

Alpha-ketoglutarate decarboxylase. The Mycobacterium bovis sucA gene encoding alpha-ketoglutarate decarboxylase was expressed in host strains. Overexpression of M. bovis sucA improved BDO production (see also WO2008/115840, WO 2009/023493, U.S. publication 2009/0047719, U.S. publication 2009/0075351). The nucleotide and amino acid sequences of M. bovis sucA and the encoded alpha-ketoglutarate decarboxylase are shown in FIG. 15.

To construct the M. bovis sucA expressing strains, fragments of the sucA gene encoding the alpha-ketoglutarate decarboxylase were amplified from the genomic DNA of Mycobacterium bovis BCG (ATCC 19015; American Type Culture Collection, Manassas VA) using primers shown below. The full-length gene was assembled by ligation reaction of the four amplified DNA fragments, and cloned into expression vectors pZS*13 and pZE23 behind the P_{A11acO-1} promoter (Lutz and Bujard, Nucleic Acids Res. 25:1203-1210 (1997)). The nucleotide sequence of the assembled gene was verified by DNA sequencing.

Primers for fragment 1:
(SEQ ID NO: 3)
5′-ATGTACCGCAAGTTCCGC-3′;
(SEQ ID NO: 4)
5′-CAATTTGCCGATGCCCAG-3′
Primers for fragment 2:
(SEQ ID NO: 5)
5′-GCTGACCACTGAAGACTTTG-3′;
(SEQ ID NO: 6)
5′-GATCAGGGCTTCGGTGTAG-3′
Primers for fragment 3:
(SEQ ID NO: 7)
5′-TTGGTGCGGGCCAAGCAGGATCTGCTC-3′;
(SEQ ID NO: 8)
5′-TCAGCCGAACGCCTCGTCGAGGATCTCCTG-3′
Primers for fragment 4:
(SEQ ID NO: 9)
5′-TGGCCAACATAAGTTCACCATTCGGGCAAAAC-3′;
(SEQ ID NO: 10)
5′-TCTCTTCAACCAGCCATTCGTTTTGCCCG-3′

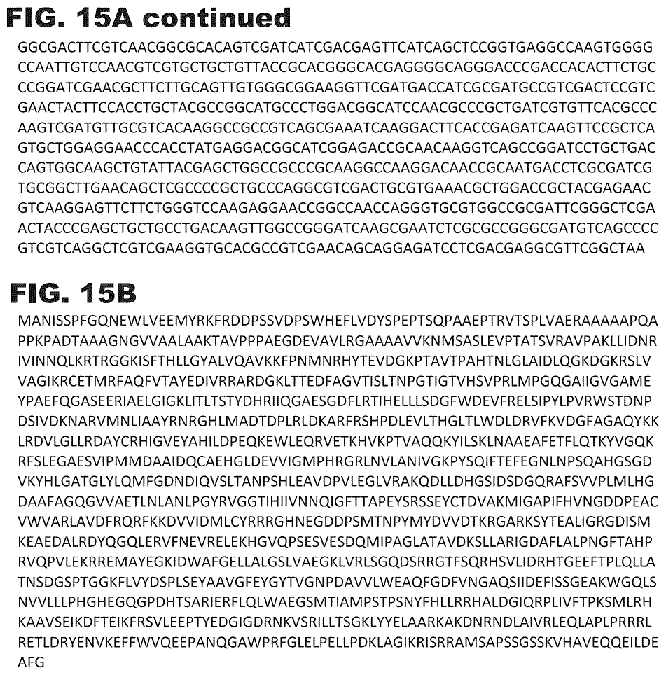

Functional expression of the alpha-ketoglutarate decarboxylase was demonstrated using both in vitro and in vivo assays. The SucA enzyme activity was measured by following a previously reported method (Tian et al., Proc. Natl. Acad. Sci. USA 102:10670-10675 (2005)). The reaction mixture contained 50 mM potassium phosphate buffer, pH 7.0, 0.2 mM thiamine pyrophosphate, 1 mM MgCl₂, 0.8 mM ferricyanide, 1 mM alpha-ketoglutarate and cell crude lysate. The enzyme activity was monitored by the reduction of ferricyanide at 430 nm. The in vivo function of the SucA enzyme was verified using E. coli whole-cell culture. Single colonies of E. coli MG1655 lacI^{q} transformed with plasmids encoding the SucA enzyme and the 4-hydroxybutyrate dehydrogenase (4Hbd) was inoculated into 5 mL of LB medium containing appropriate antibiotics. The cells were cultured at 37° C. overnight aerobically. A200 uL of this overnight culture was introduced into 8 mL of M9 minimal medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂) supplemented with 20 g/L glucose, 100 mM 3-(N-morpholino)propanesulfonic acid (MOPS) to improve the buffering capacity, 10 μg/mL thiamine, and the appropriate antibiotics. Microaerobic conditions were established by initially flushing capped anaerobic bottles with nitrogen for 5 minutes, then piercing the septum with a 23G needle following inoculation. The needle was kept in the bottle during growth to allow a small amount of air to enter the bottles. The protein expression was induced with 0.2 mM isopropyl β-D-1-thiogalactopyranoside (IPTG) when the culture reached mid-log growth phase. As controls, E. coli MG1655 lacI^{q} strains transformed with only the plasmid encoding the 4-hydroxybutyrate dehydrogenase and only the empty vectors were cultured under the same condition (see Table 17). The accumulation of 4-hydroxybutyrate (4HB) in the culture medium was monitored using LCMS method. Only the E. coli strain expressing the Mycobacterium alpha-ketoglutarate decarboxylase produced significant amount of 4HB (see FIG. 16).

TABLE 17
Three strains containing various plasmid controls
and encoding sucA and 4-hydroxybutyrate
dehydrogenase.

| | Host | pZE13 | pZA33 |
|---|---|---|---|
| 1 | MG1655 laclq | vector | vector |
| 2 | MG1655 laclq | vector | 4hbd |
| 3 | MG1655 laclq | sucA | 4hbd |

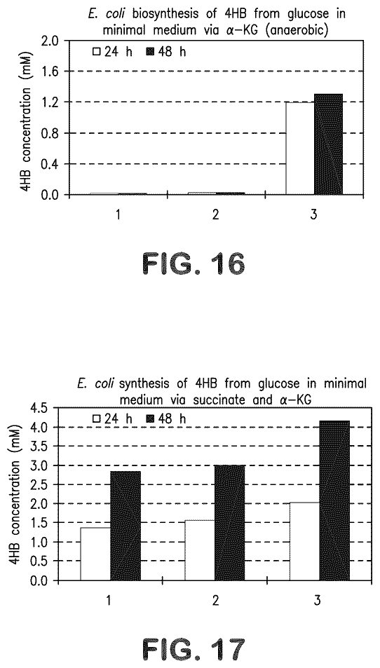

A separate experiment demonstrated that the alpha-ketoglutarate decarboxylase pathway functions independently of the reductive TCA cycle. E. coli strain ECKh-401 (ΔadhE ΔldhA ΔpflB ΔlpdA::K.p.lpdA322 Δmdh ΔarcA) was used as the host strain. All the three constructs contained the gene encoding 4HB dehydrogenase (4Hbd). Construct 1 also contained the gene encoding the alpha-ketoglutarate decarboxylase (sucA). Construct 2 contained the genes encoding the succinyl-CoA synthetase (sucCD) and the CoA-dependent succinate semialdehyde dehydrogenase (sucD), which are required for the synthesis of 4HB via the reductive TCA cycle. Construct 3 contains all the genes from 1 and 2. The three E. coli strains were cultured under the same conditions as described above except the second culture was under the micro-aerobic condition. By expressing the SucA enzyme, construct 3 produced more 4HB than construct 2, which relies on the reductive TCA cycle for 4HB synthesis (see FIG. 17).

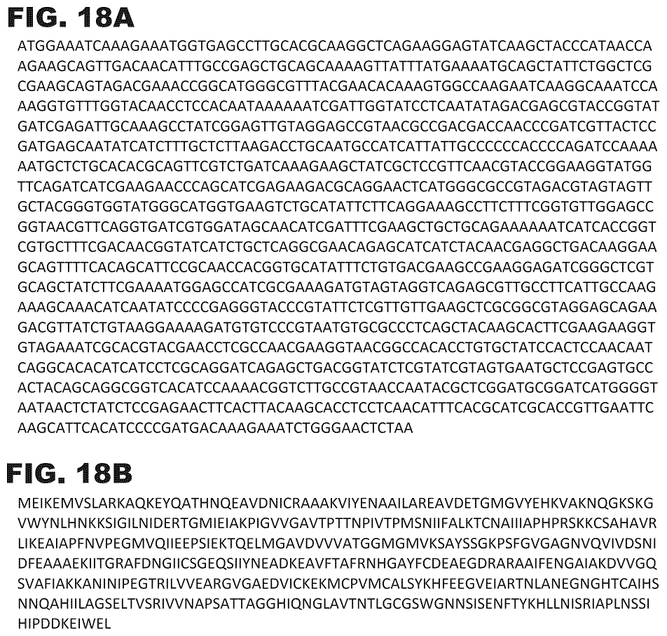

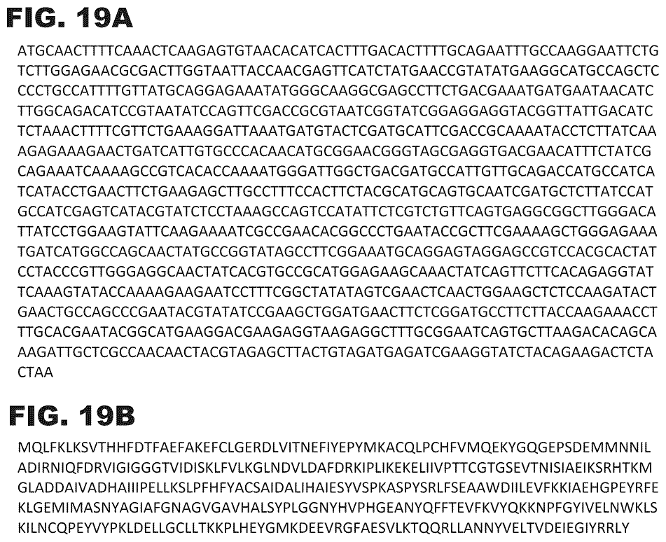

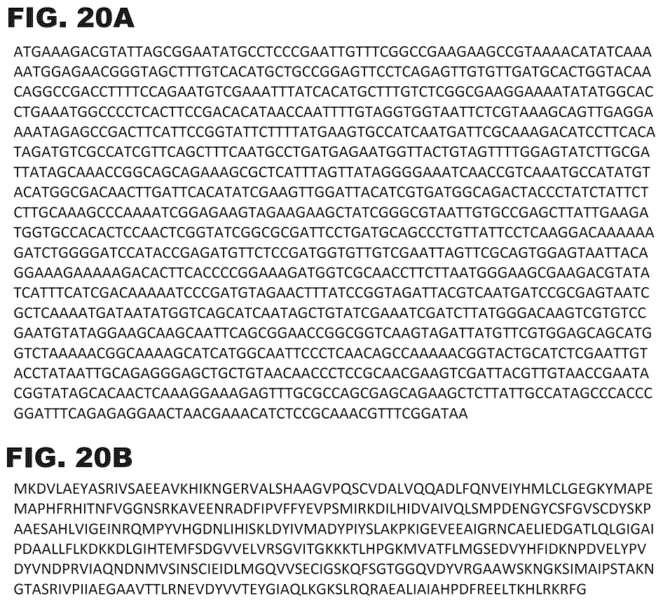

Succinate semialdehyde dehydrogenase (CoA-dependent), 4-hydroxybutyrate dehydrogenase, and 4-hydroxybutyrl-CoA/acetyl-CoA transferase. The genes from Porphyromonas gingivalis W83 can be effective components of the pathway for 1,4-butanediol production (see also WO2008/115840, WO 2009/023493, U.S. publication 2009/0047719, U.S. publication 2009/0075351). The nucleotide sequence of CoA-dependent succinate semialdehyde dehydrogenase (sucD) from Porphyromonas gingivalis is shown in FIG. 18A, and the encoded amino acid sequence is shown in FIG. 18B. The nucleotide sequence of 4-hydroxybutyrate dehydrogenase (4hbd) from Porphyromonas gingivalis is shown in FIG. 19A, and the encoded amino acid sequence is shown in FIG. 19B. The nucleotide sequence of 4-hydroxybutyrate CoA transferase (cat2) from Porphyromonas gingivalis is shown in FIG. 20A, and the encoded amino acid sequence is shown in FIG. 20B.

Briefly, the genes from Porphyromonas gingivalis W83 encoding succinate semialdehyde dehydrogenase (CoA-dependent) and 4-hydroxybutyrate dehydrogenase, and in some cases additionally 4-hydroxybutyryl-CoA/acetyl-CoA, were cloned by PCR from P. gingivalis chromosomal DNA and introduced into multicopy plasmids pZS*13, pZA13, and pZE33 behind the PAllacO-1 promoter (Lutz and Bujard, Nucleic Acids Res. 25:1203-1210 (1997)) using standard molecular biology procedures. These plasmids were then introduced into host strains.

The Porphyromonas gingivalis W83 genes were introduced into production strains as described above. Some strains included only succinate semialdehyde dehydrogenase (CoA-dependant) and 4-hydroxybutyrate dehydrogenase without 4-hydroxybutyryl-CoA/acetyl-CoA transferase.

Butyrate kinase and phosphotransbutyrylase. Butyrate kinase (BK) and phosphotransbutyrylase (PTB) enzymes can be utilized to produce 4-hydroxybutyryl-CoA (see also WO2008/115840, WO 2009/023493, U.S. publication 2009/0047719, U.S. publication 2009/0075351). In particular, the Clostridium acetobutylicum genes, buk1 and ptb, can be utilized as part of a functional BDO pathway.

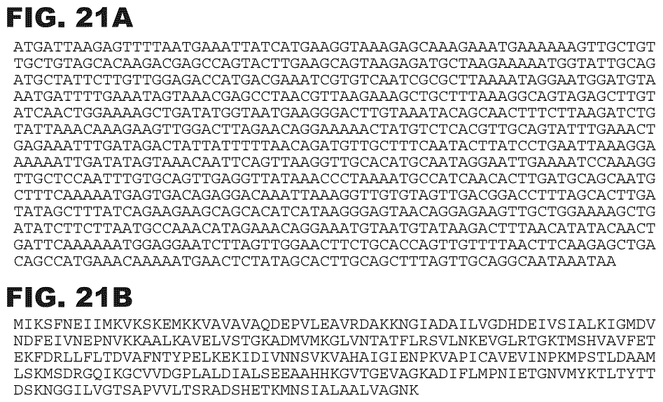

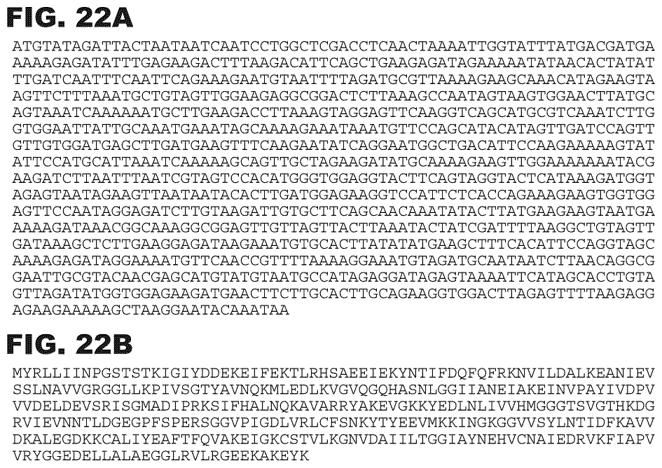

Initial experiments involved the cloning and expression of the native C. acetobutylicum PTB (020) and BK (021) genes in E. coli. Where required, the start codon and stop codon for each gene were modified to “ATG” and “TAA,” respectively, for more optimal expression in E. coli. The C. acetobutylicum gene sequences (020N and 021N) and their corresponding translated peptide sequences are shown in FIGS. 21 and 22.

The PTB and BK genes exist in C. acetobutylicum as an operon, with the PTB (020) gene expressed first. The two genes are connected by the sequence “atta aagttaagtg gaggaatgtt aac” (SEQ ID NO:11) that includes a re-initiation ribosomal binding site for the downstream BK (021) gene. The two genes in this context were fused to lac-controlled promoters in expression vectors for expression in E. coli (Lutz and Bujard, Nucleic Acids Res. 25:1203-1210 (1997)).

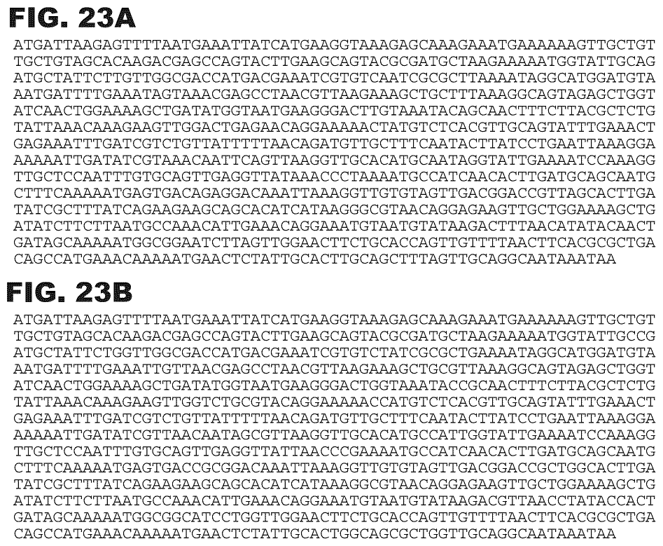

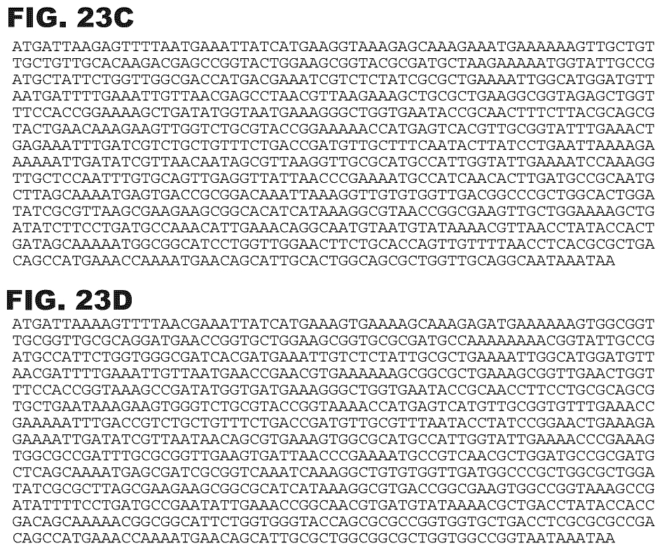

Expression of the two proteins from these vector constructs was found to be low in comparison with other exogenously expressed genes due to the high incidence of codons in the C. acetobutylicum genes that occur only rarely in E. coli. Therefore new 020 and 021 genes were predicted that changed rare codons for alternates that are more highly represented in E. coli gene sequences. This method of codon optimization followed algorithms described previously (Sivaraman et al., Nucleic Acids Res. 36:e16(2008)). This method predicts codon replacements in context with their frequency of occurrence when flanked by certain codons on either side. Alternative gene sequences for 020 (FIG. 23) and 021 (FIG. 24) were determined in which increasing numbers of rare codons were replaced by more prevalent codons (A<B<C<D) based on their incidence in the neighboring codon context. No changes in actual peptide sequence compared to the native 020 and 021 peptide sequences were introduced in these predicted sequences.

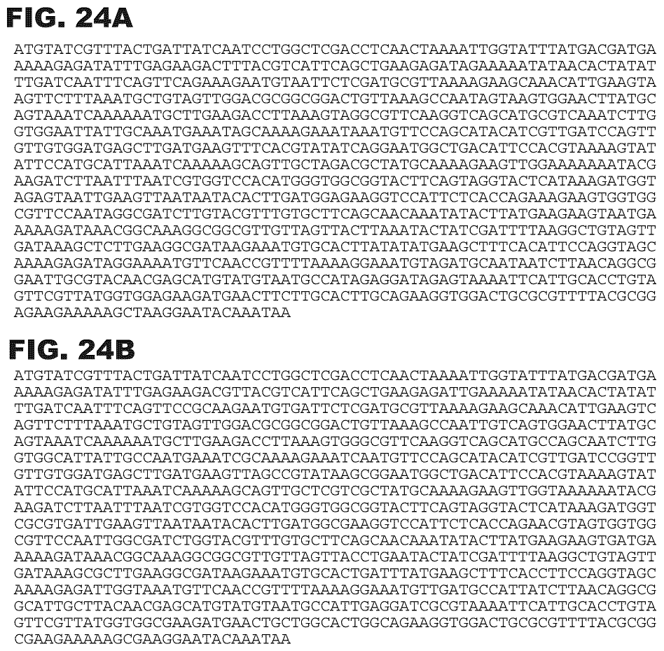

The improvement in expression of the BK and PTB proteins resulting from codon optimization is shown in FIG. 25A. Expression of the native gene sequences is shown in lane 2, while expression of the 020B-021B and 020C-021C is shown in lanes 3 and 4, respectively. Higher levels of protein expression in the codon-optimized operons 020B-021B (2021B) and 020C-021C (2021C) also resulted in increased activity compared to the native operon (2021n) in equivalently-expressed E. coli crude extracts (FIG. 25B).

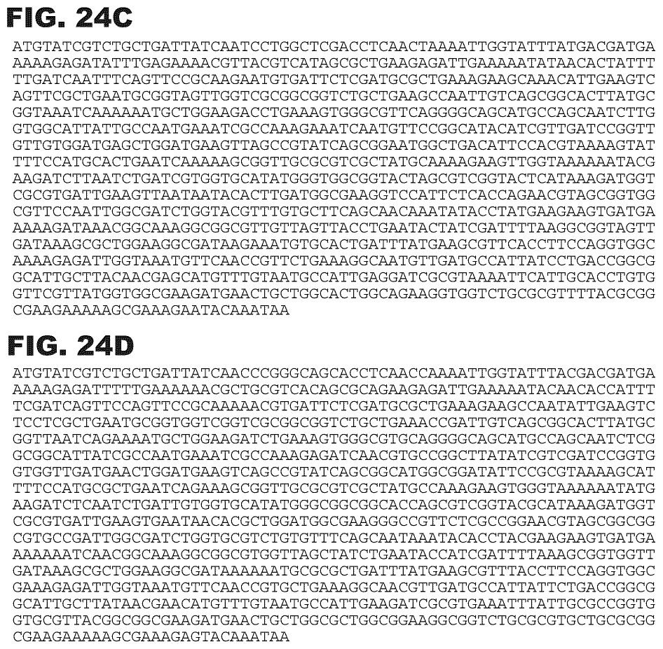

The codon optimized operons were expressed on a plasmid in strain ECKh-432 (ΔadhE ΔldhA ΔpflB ΔlpdA::K.p.lpdA322 Δmdh ΔarcA gltAR163L fimD:: E. coli sucCD, P. gingivalis sucD, P. gingivalis 4hbd fimD:: M. bovis sucA, C. kluyveri 4hbd) along with the C. acetobutylicum aldehyde dehydrogenase to provide a complete BDO pathway. Cells were cultured in M9 minimal medium containing 20 g/L glucose, using a 23G needle to maintain microaerobic conditions as described above. The resulting conversion of glucose to the final product BDO was measured. Also measured was the accumulation of gamma-butyrylactone (GBL), which is a spontaneously rearranged molecule derived from 4Hb-CoA, the immediate product of the PTB-BK enzyme pair. FIG. 26 shows that expression of the native 2021n operon resulted in comparable BDO levels to an alternative enzyme function, Cat2 (034), that is capable of converting 4HB and free CoA to 4HB-CoA. GBL levels of 034 were significantly higher than 2021n, suggesting that the former enzyme has more activity than PTB-BK expressed from the native genes. However levels of both BDO and GBL were higher than either 034 or 2021n when the codon-optimized variants 2021B and 2021C were expressed, indicating that codon optimization of the genes for PTB and BK significantly increases their contributions to BDO synthesis in E. coli.

These results demonstrate that butyrate kinase (BK) and phosphotransbutyrylase (PTB) enzymes can be employed to convert 4-hydroxybutyrate to 4-hydroxybutyryl-CoA. This eliminates the need for a transferase enzyme such as 4-hydroxybutyryl-CoA/Acetyl-CoA transferase, which would generate one mole of acetate per mol of 4-hydroxybutyryl-CoA produced. The enzymes from Clostridium acetobutylicum are present in a number of engineered strains for BDO production.

4-hydroxybutyryl-CoA reductase. The Clostridium beijerinckii ald gene can be utilized as part of a functional BDO pathway (see also WO2008/115840, WO 2009/023493, U.S. publication 2009/0047719, U.S. publication 2009/0075351). The Clostridium beijerinckii ald can also be utilized to lower ethanol production in BDO producing strains. Additionally, a specific codon-optimized ald variant (GNM0025B) was found to improve BDO production.

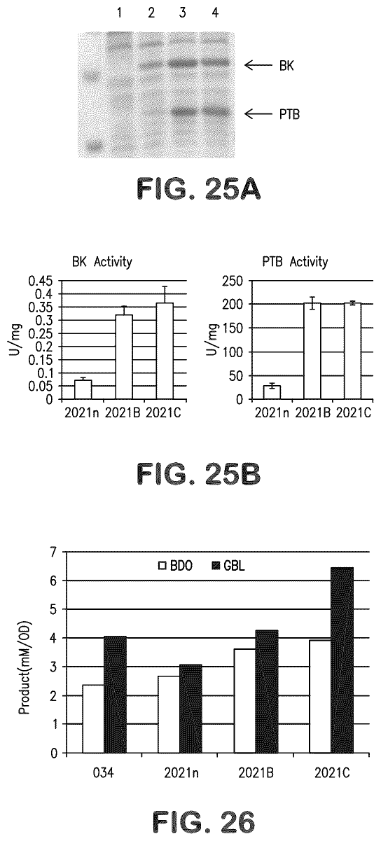

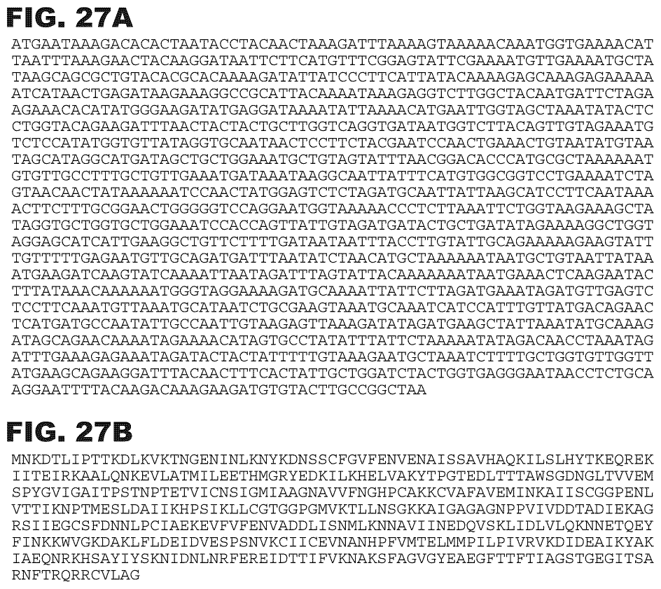

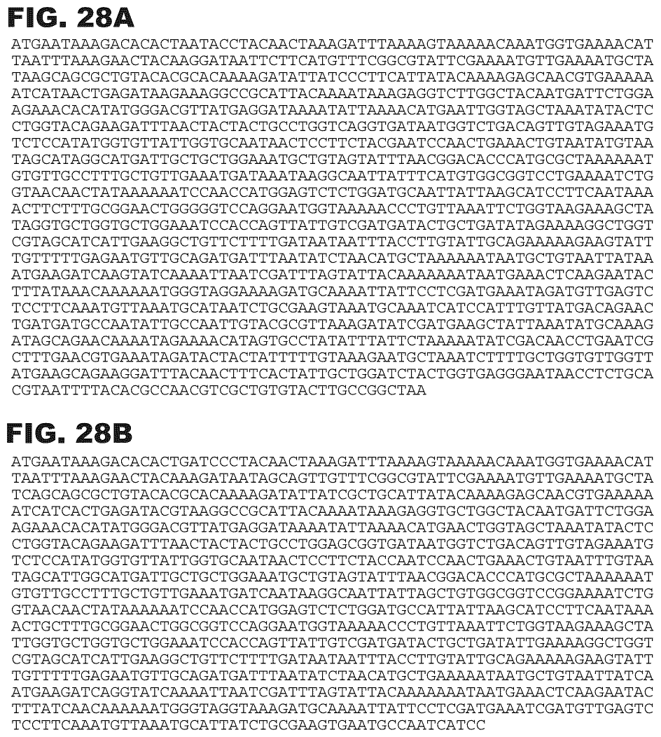

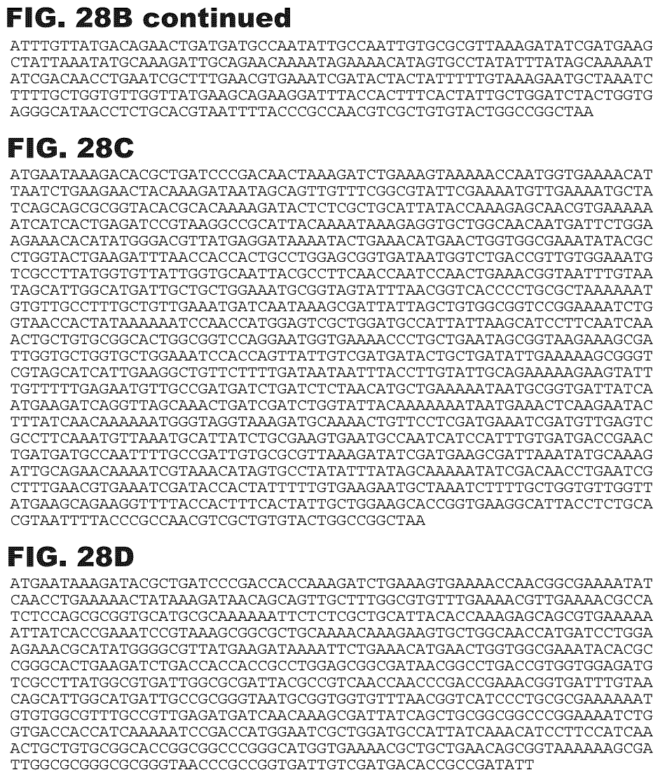

The native C. beijerinckii ald gene (025n) and the predicted protein sequence of the enzyme are shown in FIG. 27. As was seen for the Clostridium acetobutylicum PTB and BK genes, expression of the native C. beijerinckii ald gene was very low in E. coli. Therefore, four codon-optimized variants for this gene were predicted. FIGS. 28A-28D show alternative gene sequences for 025, in which increasing numbers of rare codons are replaced by more prevalent codons (A<B<C<D) based on their incidence in the neighboring codon context (25A, P=0.05; 25B, P=0.1; 25C, P=0.15; 25D, P=1). No changes in actual peptide sequence compared to the native 025 peptide sequence were introduced in these predictions. Codon optimization significantly increased expression of the C. beijerinckii ald (see FIG. 29), which resulted in significantly higher conversion of glucose to BDO in cells expressing the entire BDO pathway (FIG. 30A).

The native and codon-optimized genes were expressed on a plasmid along with P. gingivalis Cat2, in the host strain ECKh-432 (ΔadhE ΔldhA ΔpflB ΔlpdA::K.p.lpdA322 Δmdh ΔarcA gltAR163L ΔackA fimD:: E. coli sucCD, P. gingivalis sucD, P. gingivalis 4hbd fimD:: M. bovis sucA, C. kluyveri 4hbd), thus containing a complete BDO pathway. Cells were cultured microaerobically in M9 minimal medium containing 20 g/L glucose as described above. The relative production of BDO and ethanol by the C. beijerinckii Ald enzyme (expressed from codon-optimized variant gene 025B) was compared with the C. acetobutylicum AdhE2 enzyme (see FIG. 30B). The C. acetobutylicum AdhE2 enzyme (002C) produced nearly 4 times more ethanol than BDO. In comparison, the C. beijerinckii Ald (025B)(in conjunction with an endogenous ADH activity) produced equivalent amounts of BDO, yet the ratio of BDO to ethanol production was reversed for this enzyme compared to 002C. This suggests that the C. beijerinckii Ald is more specific for 4HB-CoA over acetyl-coA than the C. acetobutylicum AdhE2, and therefore the former is the preferred enzyme for inclusion in the BDO pathway.

The Clostridium beijerinckii ald gene (Toth et al., Appl. Environ. Microbiol. 65:4973-4980 (1999)) was tested as a candidate for catalyzing the conversion of 4-hydroxybutyryl-CoA to 4-hydroxybutanal. Over fifty aldehyde dehydrogenases were screened for their ability to catalyze the conversion of 4-hydroxybutyryl-CoA to 4-hydroxybutyraldehyde. The C. beijerinckii ald gene was chosen for implementation into BDO-producing strains due to the preference of this enzyme for 4-hydroxybutyryl-CoA as a substrate as opposed to acetyl-CoA. This is important because most other enzymes with aldehyde dehydrogenase functionality (for example, adhE2 from C. acetobutylicum (Fontaine et al., J Bacteriol. 184:821-830 (2002)) preferentially convert acetyl-CoA to acetaldehyde, which in turn is converted to ethanol. Utilization of the C. beijerinckii gene lowers the amount of ethanol produced as a byproduct in BDO-producing organisms. Also, a codon-optimized version of this gene expresses very well in E. coli (Sivaraman et al., Nucleic Acids Res. 36:e16 (2008)).

4-hydroxybutanal reductase. 4-hydroxybutanal reductase activity of adh1 from Geobacillus thermoglucosidasius (M10EXG) was utilized. This led to improved BDO production by increasing 4-hydroxybutanal reductase activity over endogenous levels.

Multiple alcohol dehydrogenases were screened for their ability to catalyze the reduction of 4-hydroxybutanal to BDO. Most alcohol dehydrogenases with high activity on butyraldehyde exhibited far lower activity on 4-hydroxybutyraldehyde. One notable exception is the adh1 gene from Geobacillus thermoglucosidasius M10EXG (Jeon et al., J. Biotechnol. 135:127-133 (2008))(GNM0084), which exhibits high activity on both 4-hydroxybutanal and butanal.

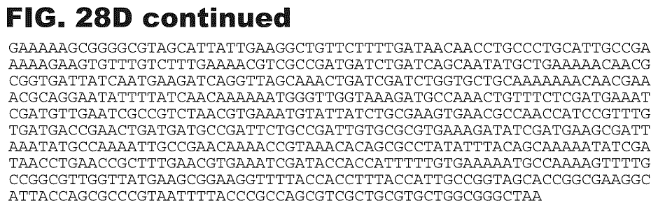

The native gene sequence and encoded protein sequence if the adh1 gene from Geobacillus thermoglucosidasius are shown in FIG. 31. The G. thermoglucosidasius ald1 gene was expressed in E. coli.

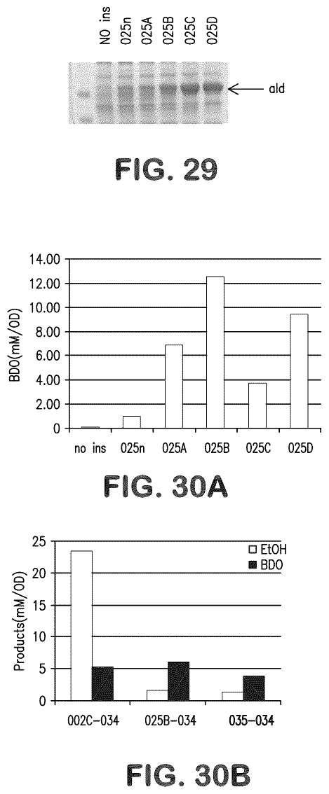

The Adh1 enzyme (084) expressed very well from its native gene in E. coli (see FIG. 32A). In ADH enzyme assays, the E. coli expressed enzyme showed very high reductive activity when butyraldehyde or 4HB-aldehyde were used as the substrates (see FIG. 32B). The Km values determined for these substrates were 1.2 mM and 4.0 mM, respectively. These activity values showed that the Adh1 enzyme was the most active on reduction of 4HB-aldehyde of all the candidates tested.

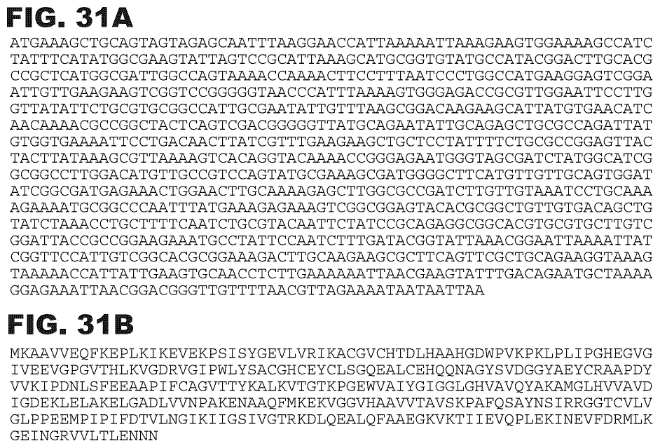

The 084 enzyme was tested for its ability to boost BDO production when coupled with the C. beijerinckii aid. The 084 gene was inserted behind the C. beijerinckii ald variant 025B gene to create a synthetic operon that results in coupled expression of both genes. Similar constructs linked 025B with other ADH candidate genes, and the effect of including each ADH with 025B on BDO production was tested. The host strain used was ECKh-459 (ΔadhE ldhA ΔpflB ΔlpdA::fnr-pflB6-K.p.lpdA322 Δmdh ΔarcA gltAR163L fimD:: E. coli sucCD, P. gingivalis sucD, P. gingivalis 4hbd fimD:: M. bovis sucA, C. kluyveri 4hbd fimD:: C. acetobutylicum buk1, C. acetobutylicumptb), which contains the remainder of the BDO pathway on the chromosome. The 084 ADH expressed in conjunction with 025B showed the highest amount of BDO (right arrow in FIG. 33) when compared with 025B only (left arrow in FIG. 33) and in conjunction with endogenous ADH functions. It also produced more BDO than did other ADH enzymes when paired with 025B, indicated as follows: 026A-C, codon-optimized variants of Clostridium acetobutylicum butanol dehydrogenase; 050, Zymomonas mobilis alcohol dehydrogenase; 052, Citrobacter freundii 1,3-propanediol dehydrogenase; 053, Lactobacillus brevis 1,3-propanediol dehydrogenase; 057, Bacteroides fragilis lactaldehyde reductase; 058, E. coli 1,3-propanediol dehydrogenase; 071, Bacillus subtilis 168 alpha-ketoglutarate semialdehyde dehydrogenase. The constructs labeled “PT5lacO” are those in which the genes are driven by the PT5lacO promoter. In all other cases, the PAllacO-1 promoter was used. This shows that inclusion of the 084ADH in the BDO pathway increased BDO production.

### Example XIV

### BDO Producing Strains Expressing Pyruvate Dehydrogenase

This example describes the utilization of pyruvate dehydrogenase (PDH) to enhance BDO production. Heterologous expression of the Klebsiella pneumonia lpdA gene was used to enhance BDO production. See corresponding example in WO2013/186402.

PDH is one of the most complicated enzymes of central metabolism and is comprised of 24 copies of pyruvate decarboxylase (E1) and 12 molecules of dihydrolipoyl dehydrogenase (E3), which bind to the outside of the dihydrolipoyl transacetylase (E2) core. PDH is inhibited by high NADH/NAD, ATP/ADP, and Acetyl-CoA/CoA ratios. The enzyme naturally exhibits very low activity under oxygen-limited or anaerobic conditions in organisms such as E. coli due in large part to the NADH sensitivity of E3, encoded by lpdA. To this end, an NADH-insensitive version of the lpdA gene from Klebsiella pneumonia was cloned and expressed to increase the activity of PDH under conditions where the NADH/NAD ratio is expected to be high.

Replacement of the native lpdA. The pyruvate dehydrogenase operon of Klebsiella pneumoniae is between 78 and 95% identical at the nucleotide level to the equivalent operon of E. coli. It was shown previously that K. pneumoniae has the ability to grow anaerobically in presence of glycerol (Menzel et al., J. Biotechnol. 56:135-142 (1997); Menzel et al., Biotechnol. Bioeng. 60:617-626 (1998)). It has also been shown that two mutations in the lpdA gene of the operon of E. coli would increase its ability to grow anaerobically (Kim et al., Appl. Environ. Microbiol. 73:1766-1771(2007); Kim et al., J. Bacteriol. 190:3851-3858 (2008)). The lpdA gene of K. pneumonia was amplified by PCR using genomic DNA (ATCC700721D) as template and the primers KP-lpdA-Bam (5′-acacgcggatccaacgtcccgg-3′)(SEQ ID NO:12) and KP-lpdA-Nhe (5′-agcggctccgctagccgcttatg-3′)(SEQ ID NO:13). The resulting fragment was cloned into the vector pCR-BluntII-TOPO (Invitrogen; Carlsbad CA), leading to plasmid pCR-KP-lpdA.

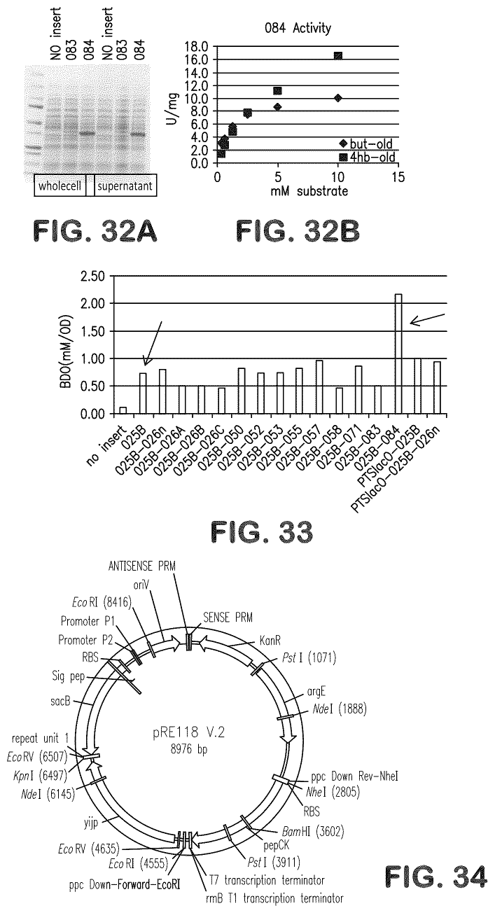

The chromosomal gene replacement was performed using a non-replicative plasmid and the sacB gene from Bacillus subtilis as a means of counterselection (Gay et al., J. Bacteriol. 153:1424-1431(1983)). The vector used is pRE118 (ATCC87693) deleted of the oriT and IS sequences, which is 3.6 kb in size and carrying the kanamycin resistance gene. The sequence was confirmed, and the vector was called pRE118-V2 (see FIG. 34).

The E. coli fragments flanking the lpdA gene were amplified by PCR using the combination of primers: EC-aceF-Pst (5′-aagccgttgctgcagctcttgagc-3′)(SEQ ID NO:14)+EC-aceF-Bam2(5′-atctccggcggtcggatccgtcg-3′)(SEQ ID NO:15) and EC-yacH-Nhe (5′-aaagcggctagccacgccgc-3′)(SEQ ID NO:16)+EC-yacH-Kpn (5′-attacacgaggtacccaacg-3′)(SEQ ID NO:17). A BamHI-XbaI fragment containing the lpdA gene of K. pneumonia was isolated from plasmid pCR-KP-lpdA and was then ligated to the above E. coli fragments digested with PstI+BamHI and NheI-KpnI respectively, and the pRE118-V2 plasmid digested with KpnI and PstI. The resulting plasmid (called pRE118-M2.11 lpdA yac) was subjected to Site Directed Mutagenesis (SDM) using the combination of primers KP-lpdA-HisTyr-F (5′-atgctggcgtacaaaggtgtcc-3′)(SEQ ID NO:18) and (5′-ggacacctttgtacgccagcat-3′)(SEQ ID NO:19) for the mutation of the His 322 residue to a Tyr residue or primers KP-lpdA-GluLys-F (5′-atcgcctacactaaaccagaagtgg-3′)(SEQ ID NO:20) and KP-lpdA-GluLys-R (5′-ccacttctggtttagtgtaggcgat-3′)(SEQ ID NO:21) for the mutation of the residue au 354 to Lys residue. PCR was performed with the Polymerase Pfu Turbo (Stratagene; San Diego CA). The sequence of the entire fragment as well as the presence of only the desired mutations was verified. The resulting plasmid was introduced into electro competent cells of E. coli ΔadhE::Frt-ΔldhA::Frt by transformation. The first integration event in the chromosome was selected on LB agar plates containing Kanamycin (25 or 50 mg/L). Correct insertions were verified by PCR using 2 primers, one located outside the region of insertion and one in the kanamycin gene (5′-aggcagttccataggatggc-3′)(SEQ ID NO:22). Clones with the correct insertion were selected for resolution. They were sub-cultured twice in plain liquid LB at the desired temperature and serial dilutions were plated on LB-no salt-sucrose 10% plates. Clones that grew on sucrose containing plates were screened for the loss of the kanamycin resistance gene on LB-low salt agar medium and the lpdA gene replacement was verified by PCR and sequencing of the encompassing region. Sequence of the insertion region was verified, and is as described below. One clone (named 4-4-P1) with mutation Glu354Lys was selected. This clone was then transduced with P1 lysate of E. coli ΔPflB::Frt leading to strain ECKh-138 (ΔadhE ΔldhA ΔpflB ΔlpdA::K.p.lpdA322).

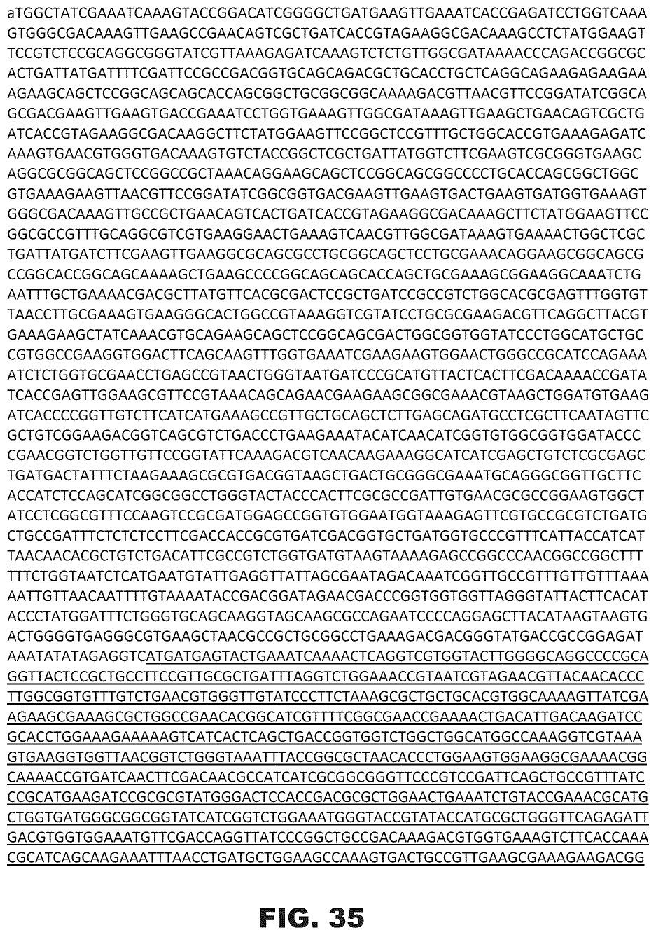

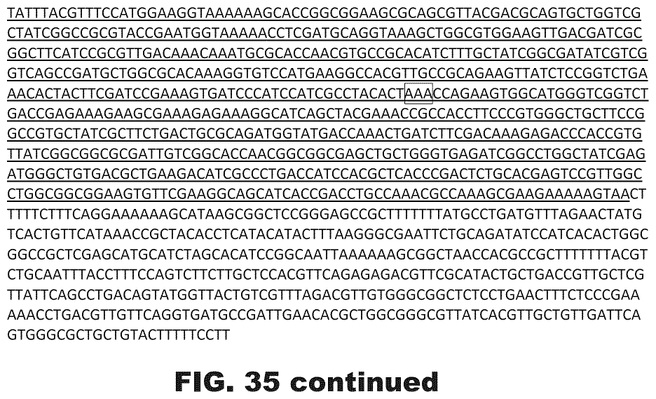

The sequence of the ECKh-138 region encompassing the aceF and lpdA genesis shown in FIG. 35. The K. pneumonia lpdA gene is underlined, and the codon changed in the Glu354Lys mutant shaded. The protein sequence comparison of the native E. coli lpdA and the mutant K. pneumonia lpdA is shown in FIG. 36.

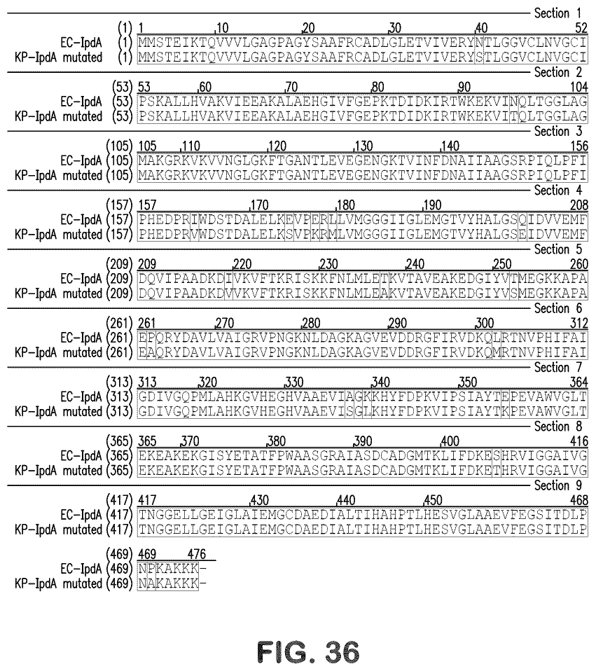

To evaluate the benefit of using K. pneumoniae lpdA in a BDO production strain, the host strains AB3 and ECKh-138 were transformed with plasmids expressing the entire BDO pathway from strong, inducible promoters. Specifically, E. coli sucCD, P. gingivalis sucD, P. gingivalis 4hbd were expressed on the medium copy plasmid pZA33, and P. gingivalis Cat2 and C. acetobutylicum AdhE2 were expressed on the high copy plasmid pZE13. These plasmids have been described in the literature (Lutz and H. Bujard, Nucleic Acids Res 25:1203-1210 (1997)), and their use for BDO pathway expression is described in Example XIII and WO2008/115840. BDO and 4HB production in ECKh-138 was significantly higher after 48 hours than in AB3 or the host used in previous work, MG1655 ΔldhA (FIG. 37).

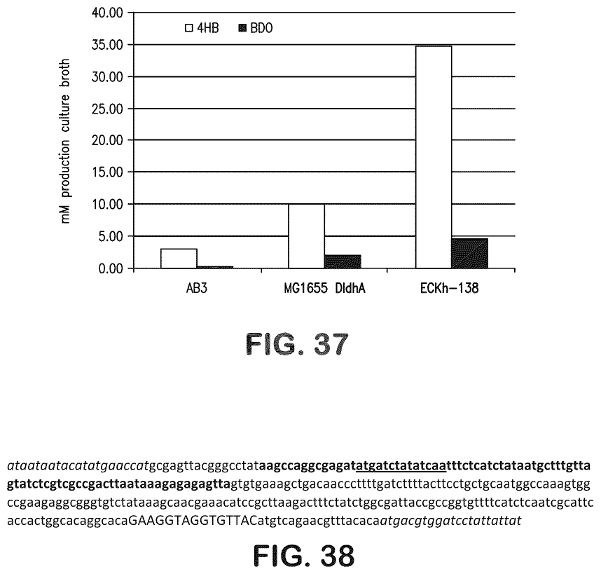

PDH promoter replacement. It was previously shown that the replacement of the pdhR repressor by a transcriptional fusion containing the Fnr binding site, one of the pflB promoters, and its ribosome binding site (RBS), thus leading to expression of the aceEF-lpd operon by an anaerobic promoter, should increase pdh activity anaerobically (Zhou et al., Biotechnol. Lett. 30:335-342(2008)). A fusion containing the Fnr binding site, the pflB-p6 promoter and an RBS binding site were constructed by overlapping PCR. Two fragments were amplified, one using the primers aceE-upstream-RC (5′-tgacatgtaacacctaccttctgtgcctgtgccagtggttgctgtgatatagaag-3′)(SEQ ID NO:23) and pflBp6-Up-Nde (5′-ataataatacatatgaaccatgcgagttacgggcctataagccaggcg-3′)(SEQ ID NO:24) and the other using primers aceE-EcoRV-EC (5′-agtttttcgatatctgcatcagacaccggcacattgaaacgg-3′)(SEQ ID NO:25) and aceE-upstream (5′-ctggcacaggcacagaaggtaggtgttacatgtcagaacgtttacacaatgacgtggatc-3′)(SEQ ID NO:26). The tw fragments were assembled by overlapping PCR, and the final DNA fragment was digested with the restriction enzymes NdeI and BamHI. This fragment was subsequently introduced upstream of the aceE gene of the E. coli operon using pRE118-V2 as described above. The replacement was done in strains ECKh-138 and ECKh-422. The nucleotide sequence encompassing the 5′ region of the aceE gene was verified and is shown in FIG. 38. FIG. 38 shows the nucleotide sequence of 5′ end of the aceE gene fused to the pflB-p6 promoter and ribosome binding site (RBS). The 5′ italicized sequence shows the start of the aroP gene, which is transcribed in the opposite direction from the pdh operon. The 3′ italicized sequence shows the start of the aceE gene. In upper case: pflB RBS. Underlined: FNR binding site. In bold: pflB-p6 promoter sequence.

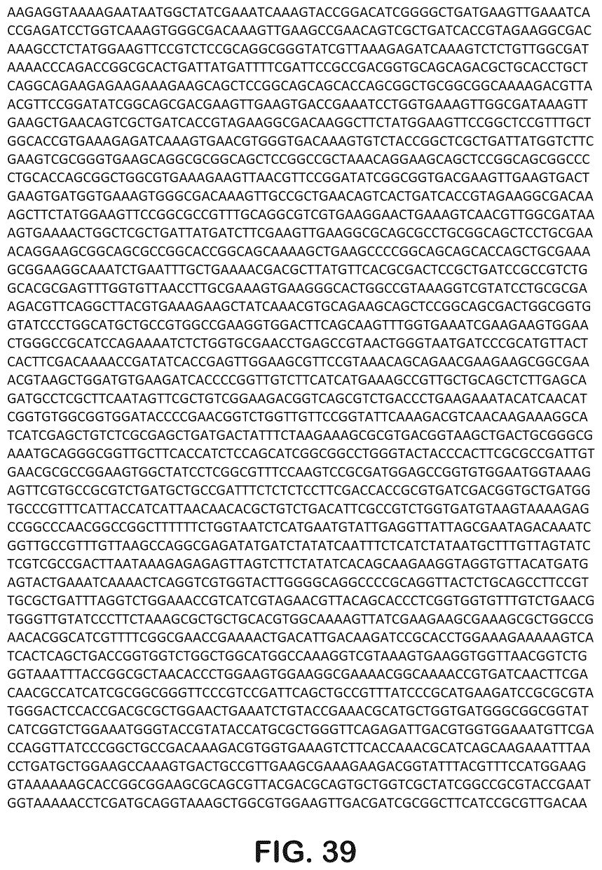

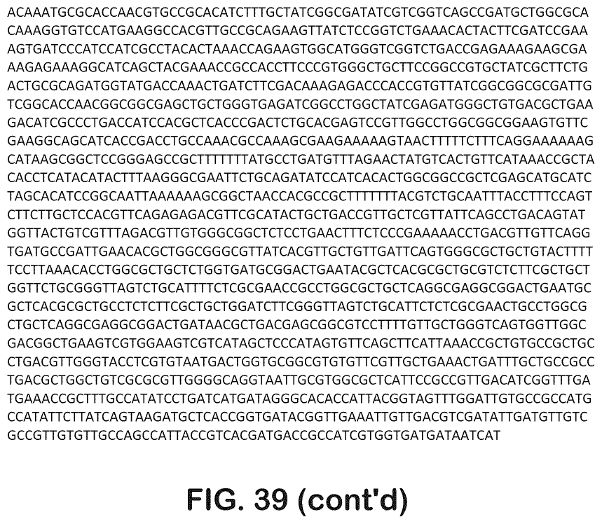

lpdA promoter replacement. The promoter region containing the fnr binding site, the pflB-p6 promoter and the RBS of the pflB gene was amplified by PCR using chromosomal DNA template and primers aceF-pflBp6-fwd (5′-agacaaatcggttgccgtttgttaagccaggcgagatatgatctatatc-3′)(SEQ ID NO:27) and lpdA-RBS-B-rev (5′-gagttttgatttcagtactcatcatgtaacacctaccttcttgctgtgatatag-3′)(SEQ ID NO:28). Plasmid 2-4a was amplified by PCR using primers B-RBS-lpdAfwd (5′-ctatatcacagcaagaaggtaggtgttacatgatgagtactgaaatcaaaatc-3′)(SEQ ID NO:29) and pflBp6-aceF-rev (5′-gatatagatcatatctcgcctggcttaacaaacggcaaccgatttgtct-3′)(SEQ ID NO:30). The two resulting fragments were assembled using the BPS cloning kit (BPS Bioscience; San Diego CA). The resulting construct was sequenced verified and introduced into strain ECKh-439 using the pRE118-V2 method described above. The nucleotide sequence encompassing the aceF-lpdA region in the resulting strain ECKh-456 is shown in FIG. 39. 48 hours after induction with 0.2 mM IPTG, the concentrations of BDO, 4HB, and pyruvate were as shown in FIG. 40.

These results demonstrated that expression of pyruvate dehydrogenase increased production of BDO in BDO producing strains.

### Example XV

### BDO Producing Strains Expressing Citrate Synthase and Aconitase

This example describes increasing activity of citrate synthase and aconitase to increase production of BDO. An R163L mutation into gltA was found to improve BDO production. Additionally, an arcA knockout was used to improve BDO production. See corresponding example in WO2013/186402.

The following oligonucleotides were used to PCR amplify the chloramphenicol resistance gene (CAT) flanked by FRT sites from pKD3:

S-mdh-Kan
(SEQ ID NO: 31)
5′-TAT TGT GCA TAC AGA TGA ATT TTT ATG CAA ACA GTC
AGC CCT GAA GAA GGG TGT AGG CTG GAG CTG CTT C-3′
AS-mdh-Kan
(SEQ ID NO: 32)
5′-CAA AAA ACC GGA GTC TGT GCT CCG GTT TTT TAT TAT
CCG CTA ATC AAT TAC ATA TGA ATA TCC TCC TTA G-3′.

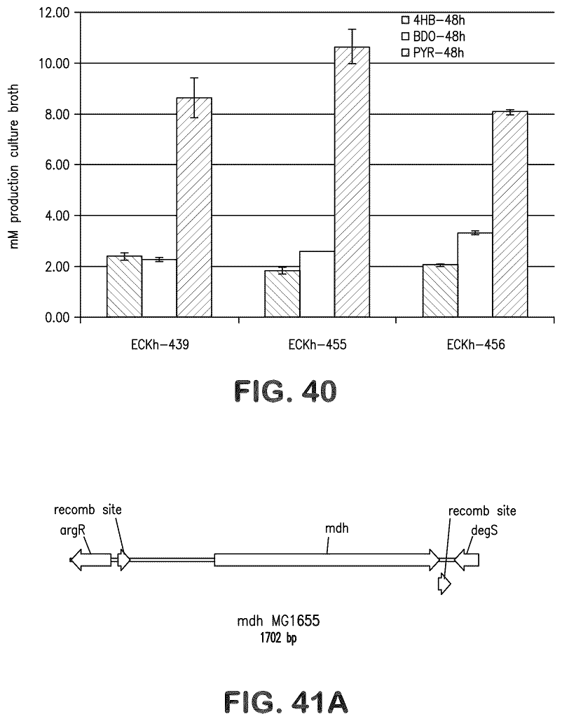

The PCR product was designed so that it integrated into the ECKh-138 genome at a region upstream of the mdh gene, as shown in FIG. 41.

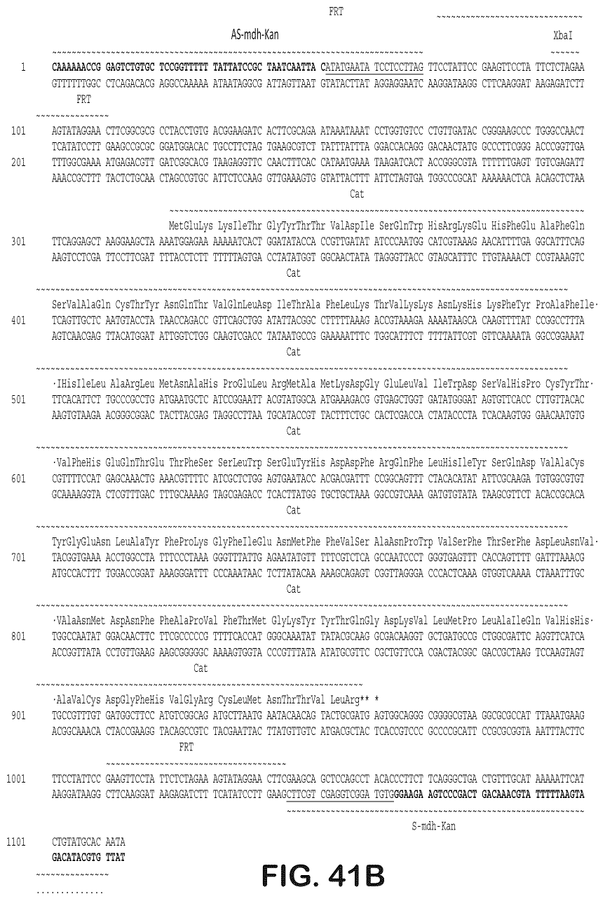

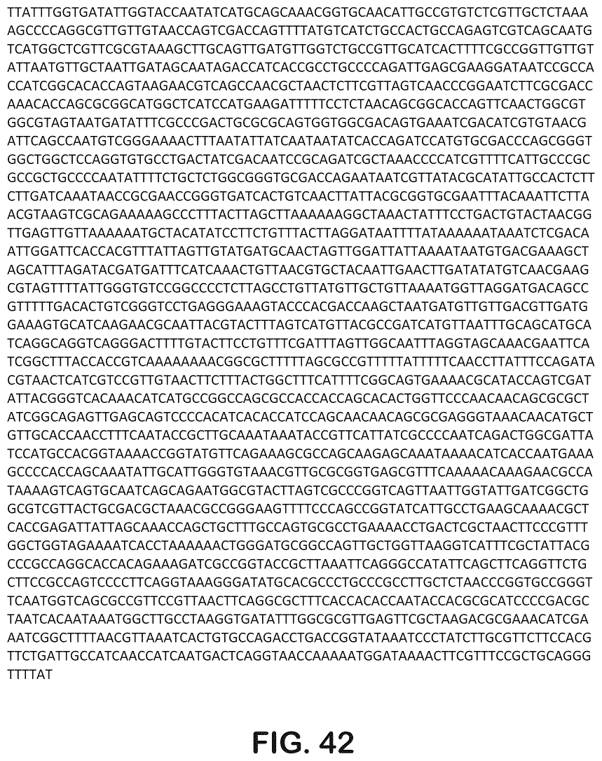

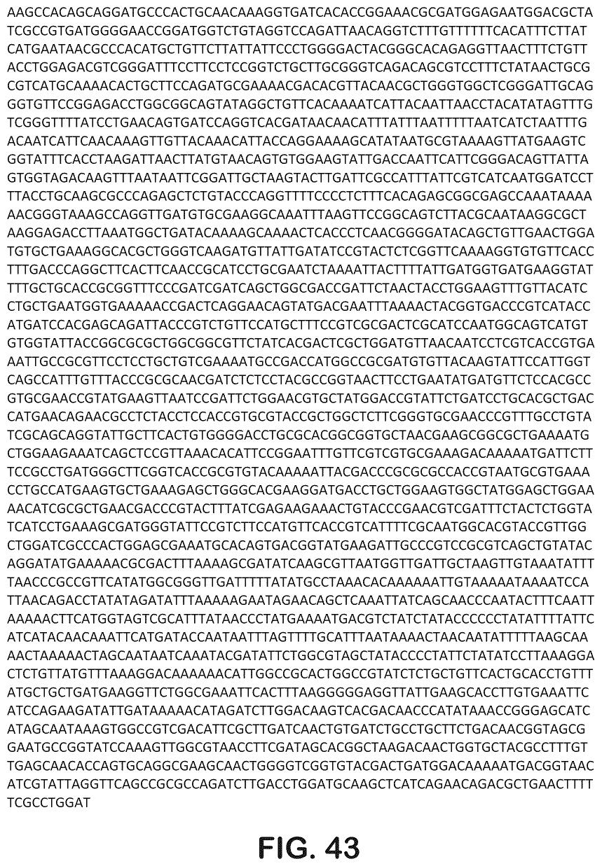

The upstream and downstream regions of the arcA gene of E. coli MG1655 were amplified by PCR using primers ArcA-up-EcoRI (5′-ataataatagaattcgtttgctacctaaattgccaactaaatcgaaacagg-3′)(SEQ ID NO:33) with ArcA-up-KpnI (5′-tattattatggtaccaatatcatgcagcaaacggtgcaacattgccg-3′)(SEQ ID NO:34) and ArcA-down-EcoRI (5′-tgatctggaagaattcatcggctttaccaccgtcaaaaaaaacggcg-3′)(SEQ ID NO:35) with ArcA-down-PstI (5′-ataaaaccctgcagcggaaacgaagttttatccatttttggttacctg-3′)(SEQ ID NO:36), respectively. After integration and resolution on LB-no salt-sucrose plates as described above, the deletion of the arcA gene in the chromosome of the resulting strain ECKh-401 was verified by sequencing and is shown in FIG. 42. The gltA gene of E. coli MG1655 was amplified by PCR using primers gltA-up (5′-ggaagagaggctggtacccagaagccacagcagga-3′)(SEQ ID NO:37) and gltA-PstI (5′-gtaatcactgcgtaagcgccatgccccggcgttaattc-3′)(SEQ ID NO:38). The amplified fragment was cloned into pRE118-V2 after digestion with KpnI and PstI. The resulting plasmid was called pRE118-gltA. This plasmid was then subjected to site directed mutagenesis (SDM) using primers R163L-f(5′-attgccgcgttcctcctgctgtcga-3′)(SEQ ID NO:39) and R163L-r (5′-cgacagcaggaggaacgcggcaat-3′)(SEQ ID NO:40) to change the residue Arg 163 to a Lys residue. The sequence of the entire fragment was verified by sequencing The resulting strain was called ECKh-422, and has the genotype ΔadhE ΔldhA ΔpflB ΔlpdA::K.p.lpdA322 Δmdh ΔarcA gltAR163L. The region encompassing the mutated gltA gene of strain ECKh-422 was verified by sequencing, as shown in FIG. 43. FIG. 44 shows the citrate synthase activity of wild type gltA gene product and the R163L mutant. The assay was performed in the absence or presence of 0.4 mM NADH.

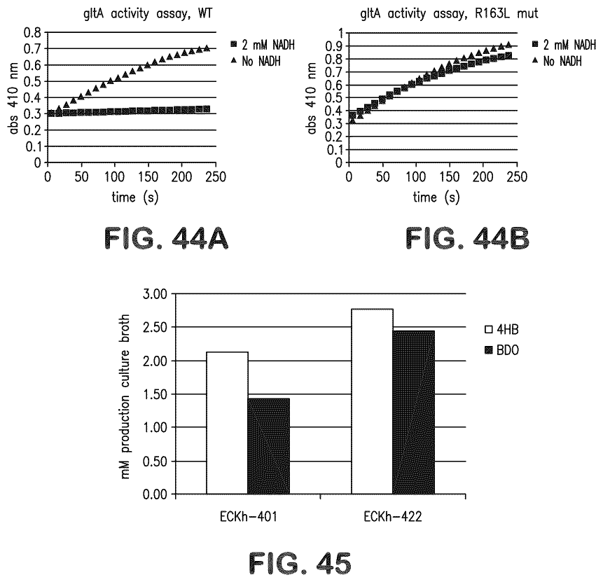

Strains ECKh-401 and ECKh-422 were transformed with plasmids expressing the entire BDO pathway. E. coli sucCD, P. gingivalis sucD, P. gingivalis 4hbd, and M. bovis sucA were expressed on the low copy plasmid pZS*13, and P. gingivalis Cat2 and C. acetobutylicum AdhE2 were expressed on the medium copy plasmid pZE23. Cultures of these strains were grown microaerobically in M9 minimal medium supplemented with 20 g/L glucose and the appropriate antibiotics as described above. The 4HB and BDO concentrations at 48 hours post-induction averaged from duplicate cultures are shown in FIG. 45. Both are higher in ECKh-422 than in ECKh-401, demonstrating that the enhanced citrate synthase activity due to the gltA mutation results in increased flux to the BDO pathway.

The host strain modifications described in this section were intended to redirect carbon flux through the oxidative TCA cycle, which is consistent with the OptKnock strain design described in WO 2009/023493 and U.S. publication 2009/0047719. To demonstrate that flux was indeed routed through this pathway, ¹³C flux analysis was performed using the strain ECKh-432, which is a version of ECKh-422 in which the upstream pathway is integrated into the chromosome (as described in Example XVII). To complete the BDO pathway, P. gingivalis Cat2 and C. beijerinckii Ald were expressed from pZS*13. Four parallel cultures were grown in M9 minimal medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂) containing 4 g/L total glucose of four different labeling ratios (¹⁻¹³C, only the first carbon atom in the glucose molecule is labeled with ¹³C; uniform-¹³C, all carbon atoms are ¹³C)
1. 80 mol % unlabeled, 20 mol % uniform-¹³C
2. 10 mol % unlabeled, 90 mol % uniform-¹³C
3. 90 mol % ¹⁻¹³C, 10 mol % uniform-¹³C
4. 40 mol % ¹⁻¹³C, 60 mol % uniform-¹³C

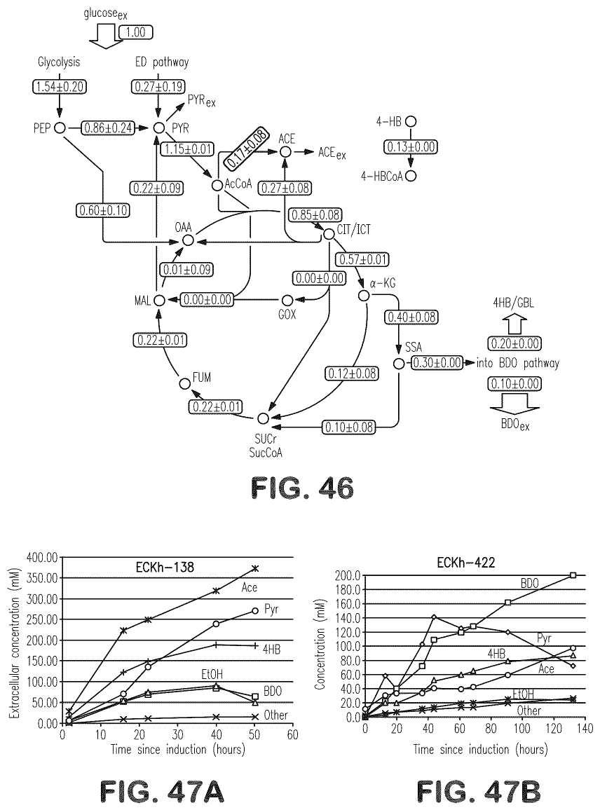

Parallel unlabeled cultures were grown in duplicate, from which frequent samples were taken to evaluate growth rate, glucose uptake rate, and product formation rates. In late exponential phase, the labeled cultures were harvested, the protein isolated and hydrolyzed to amino acids, and the label distribution of the amino acids analyzed by gas chromatography-mass spectrometry (GCMS) as described previously (Fischer and Sauer, Eur. J. Biochem. 270:880-891 (2003)). In addition, the label distribution of the secreted 4HB and BDO in the broth from the labeled cultures was analyzed by GCMS as described in WO2008115840. This data was collectively used to calculate the intracellular flux distribution using established methods (Suthers et al., Metab. Eng. 9:387-405 (2007)). The resulting central metabolic fluxes and associated 95% confidence intervals are shown in FIG. 46. Values are molar fluxes normalized to a glucose uptake rate of 1 mmol/hr. The result indicates that carbon flux is routed through citrate synthase in the oxidative direction, and that most of the carbon enters the BDO pathway rather than completing the TCA cycle. Furthermore, it confirms there is essentially no flux between malate and oxaloacetate due to the md deletion in this strain.

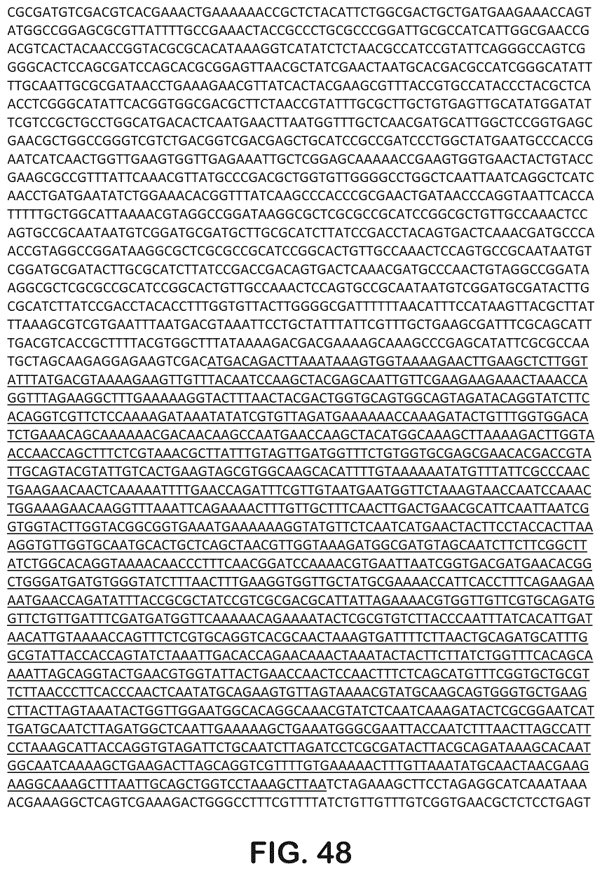

The advantage of using a knockout strain such as strains designed using OptKnock for BDO production (see WO 2009/023493 and U.S. publication 2009/0047719) can be observed by comparing typical fermentation profiles of ECKh-422 with that of the original strain ECKh-138, in which BDO is produced from succinate via the reductive TCA cycle (see FIG. 47). Fermentations were performed with 1 L initial culture volume in 2 L Biostat B+ bioreactors (Sartorius; Cedex France) using M9 minimal medium supplemented with 20 g/L glucose. The temperature was controlled at 37° C., and the pH was controlled at 7.0 using 2 M NH₄OH or Na₂CO₃. Cells were grown aerobically to an OD600 of approximately 10, at which time the cultures were induced with 0.2 mM IPTG. One hour following induction, the air flow rate was reduced to 0.02 standard liters per minute for microaerobic conditions. The agitation rate was set at 700 rpm. Concentrated glucose was fed to maintain glucose concentration in the vessel between 0.5 and 10 g/L. Both strains were transformed with plasmids bearing the entire BDO pathway, as in the examples above. In ECKh-138, acetate, pyruvate, and 4HB dominate the fermentation, while with ECKh-422 BDO is the major product.

### Example XVI

### BDO Strains Expression Phosphoenolpyruvate Carboxykinase

This example describes the utilization of phosphoenolpyruvate carboxykinase (PEPCK) to enhance BDO production. The Haemophilus influenza PEPCK gene was used for heterologous expression. See corresponding example in WO2013/186402.

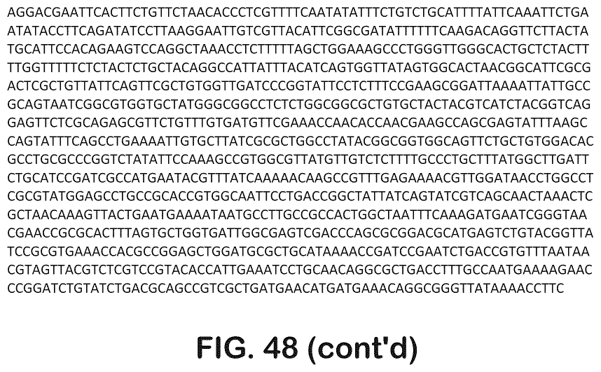

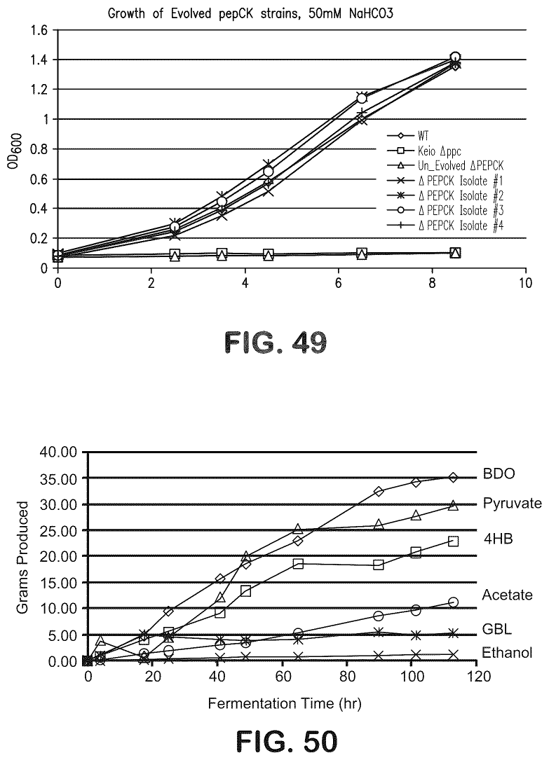

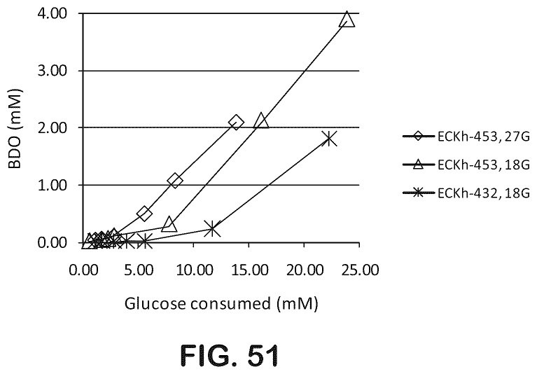

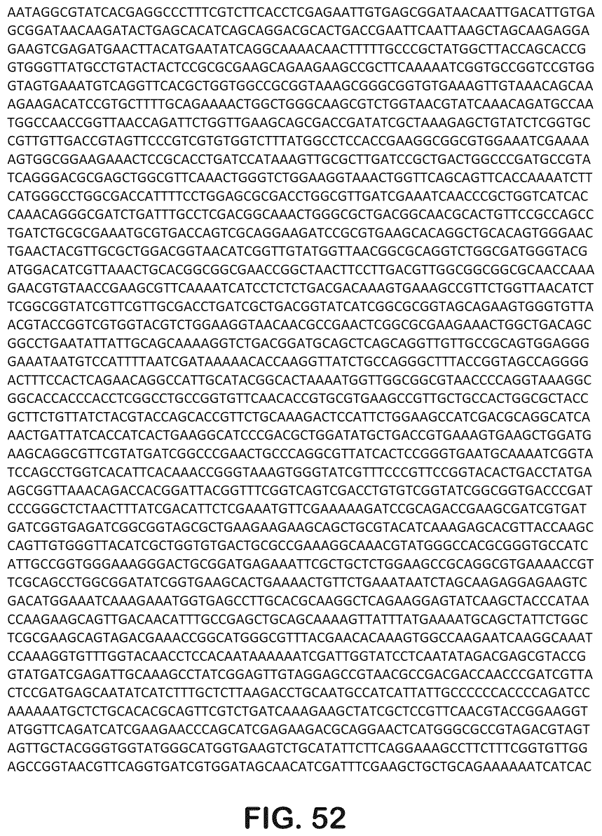

Briefly, three non native PEPCK enzymes were tested for their ability to complement growth of a PPC mutant strain of E. coli in glucose minimal media: Haemophilus influenza (NC 000907.1); Actinobacillus succinogenes (YP_001343536.1); and Mannheimia succiniciproducens (YP_089485.1). Haemophilus influenza PEPCK was integrated into the PPC locus of wild-type E. coli (MG1655)(sequence of this region shown in FIG. 48 (pepck coding region underlined). Techniques for adaptive evolution were applied to improve the growth rate of the E. coli mutant (Δppc::H. inf pepCK). Following evolution, individual colonies were isolated, and growth in anaerobic bottles was compared to that of the initial mutant and wild-type strain (see FIG. 49). The ppc/pepck gene replacement procedure described above was then repeated, this time using the BDO-producing strains ECKh-432 (ΔadhE ΔldhA ΔpflB ΔlpdA::K.p.lpdA322 Δmdh ΔarcA gltAR163L ΔackA fimD:: E. coli sucCD, P. gingivalis sucD, P. gingivalis 4hbd fimD:: M. bovis sucA, C. kluyveri 4hbd) and ECKh-439 as the hosts. The Δppc::H. inf pepCK derivative of ECKh-439, called ECKh-453, was run in a fermentation. The downstream BDO pathway was supplied by pZS*13 containing P. gingivalis Cat2 and C. beijerinckii Ald. The product profile is shown in FIG. 50. The growth coupling potential of ECKh-432 and ECKh-453 was evaluated by growth in microaerobic bottles with frequent sampling during the exponential phase. As shown in FIG. 51, ECKh-432 does not begin producing BDO until 5 g/L glucose has been consumed, corresponding to the onset of stationary phase. ECKh-453 produces BDO more evenly throughout the experiment.

### Example XVII

### Integration of BDO Pathway Encoding Genes at Specific Integration Sites

This example describes integration of various BDO pathway genes into the fimD locus to provide more efficient expression and stability. See corresponding example in WO2013/186402.

The entire upstream BDO pathway, leading to 4HB, has been integrated into the E. coli chromosome at the fimD locus. A polycistronic DNA fragment containing a promoter, the sucCD gene, the sucD gene and the 4hbd gene and a terminator sequence was inserted into the AflIII site of the pKD3 plasmid. The following primers were used to amplify the operon together with the chloramphenicol marker from the plasmid. The underlined sequences are homologeous to the target insertion site.

(SEQ ID NO: 41)
5′-GTTTGCACGCTATAGCTGAGGTTGTTGTCTTCCAGCAA
CGTACCGTATACAATAGGCGTATCACGAGGCCCTTTC-3′;
(SEQ ID NO: 42)
5′-GCTACAGCATGTCACACGATCTCAACGGTCGGATGAC
CAATCTGGCTGGTATGGGAATTAGCCATGGTCC-3′.

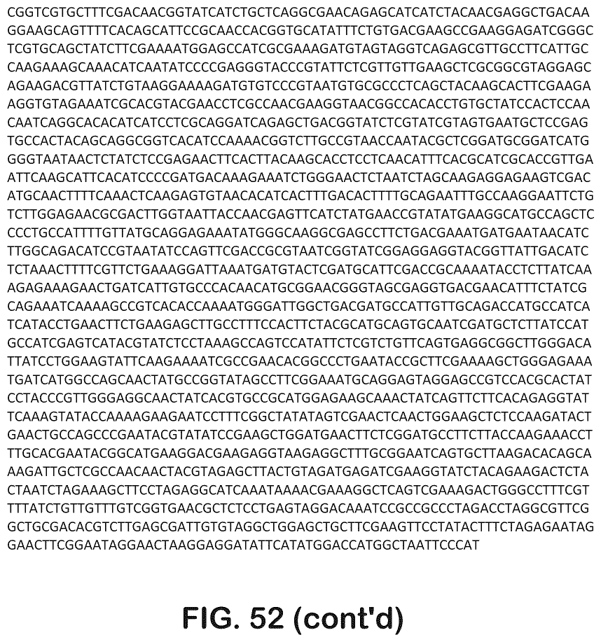

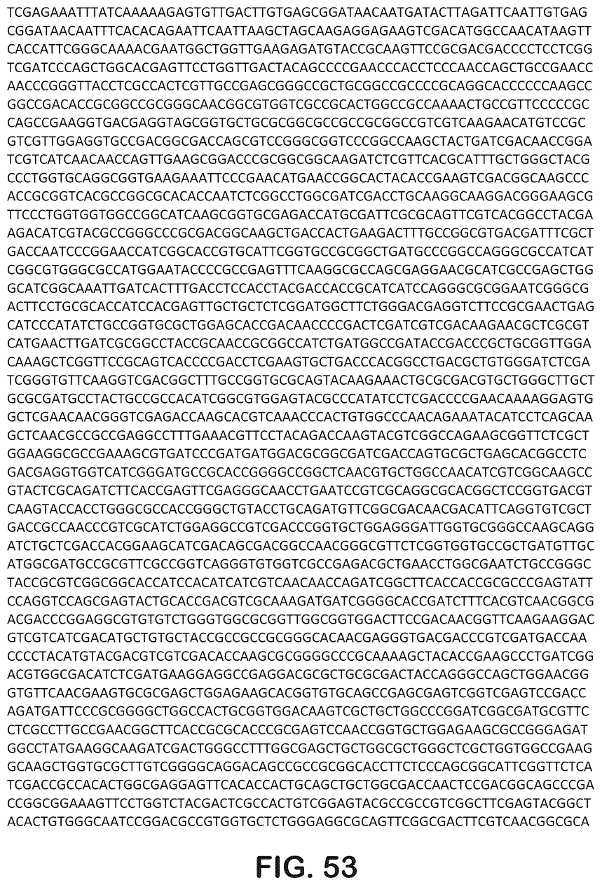

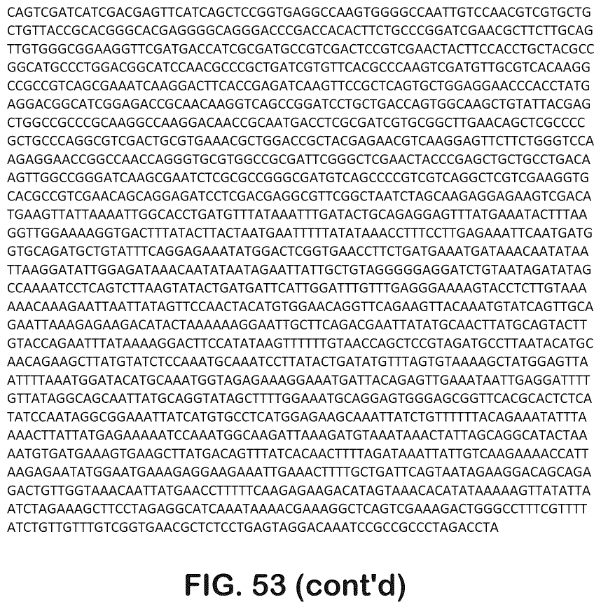

The nucleotide sequence of the region after insertion and marker removal is shown in FIG. 52. The alpha-ketoglutarate branch of the upstream pathway was integrated into the chromosome by homologeous recombination. The integration plasmid also contained a polycistronic sequence with a promoter, the sucA gene, the C. kluyveri 4hbd gene, and a terminator being inserted between two 1.5-kb DNA fragments that are homologeous to the flanking regions of the target insertion site. The nucleotide sequence of the chromosomal region after insertion and marker removal is shown in FIG. 53. The resulting upstream pathway integration strain ECKh-432 was transformed with a plasmid harboring the downstream pathway genes. The construct was able to produce BDO from glucose in minimal medium (see FIG. 54).

### Example XVIII

### Use of a Non-Phosphotransferase Sucrose Uptake System to Reduce Pyruvate Byproduct Formation

This example describes the utilization of a non-phosphotransferase (PTS) sucrose uptake system to reduce pyruvate as a byproduct in the conversion of sucrose to BDO. See corresponding example in WO2013/186402.

Strains engineered for the utilization of sucrose via a phosphotransferase (PTS) system produce significant amounts of pyruvate as a byproduct. Therefore, the use of a non-PTS sucrose system can be used to decrease pyruvate formation because the import of sucrose would not be accompanied by the conversion of phosphoenolpyruvate (PEP) to pyruvate. This will increase the PEP pool and the flux to oxaloacetate through PPC or PEPCK.

Insertion of a non-PTS sucrose operon into the rrnC region was performed. To generate a PCR product containing the non-PTS sucrose genes flanked by regions of homology to the rrnC region, two oligos were used to PCR amplify the csc genes from Mach1™ (Invitrogen, Carlsbad, CA). This strain is a descendent of W strain which is an E. coli strain known to be able to catabolize sucrose (Orencio-Trejo et al., Biotechnology Biofuels 1:8 (2008)). The sequence was derived from E. coli W strain KO11 (accession AY314757)(Shukla et al., Biotechnol. Lett. 26:689-693 (2004)) and includes genes encoding a sucrose permease (cscB), D-fructokinase (cscK), sucrose hydrolase (cscA), and a LacI-related sucrose-specific repressor (cscR)(see also CAA57217.1 for cscB, CAA57219.1 for cscA, and CAA57218.1 for cscK). The first 53 amino acids of cscR was effectively removed by the placement of the AS primer. The sequences of the oligos were:

rrnC 23S del S-CSC
(SEQ ID NO: 43)
5′-TGT GAG TGA AAG TCA CCT GCC TTA ATA TCT CAA AAC
TCA TCT TCG GGT GAC GAA ATA TGG CGT GAC TCG ATA
C-3′
and
rrnC 23S del AS-CSC
(SEQ ID NO: 44)
5′-TCT GTA TCA GGC TGA AAA TCT TCT CTC ATC CGC CAA
AAC AGC TTC GGC GTT AAG ATG CGC GCT CAA GGA C-3′.

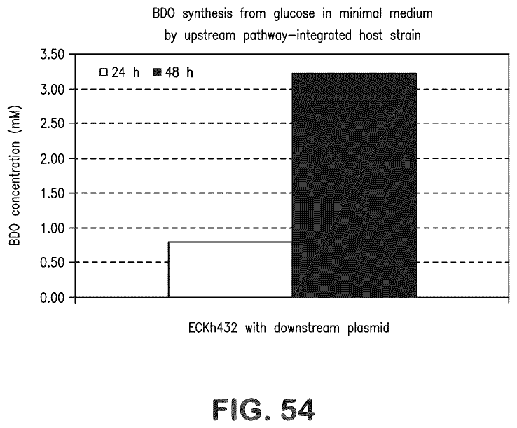

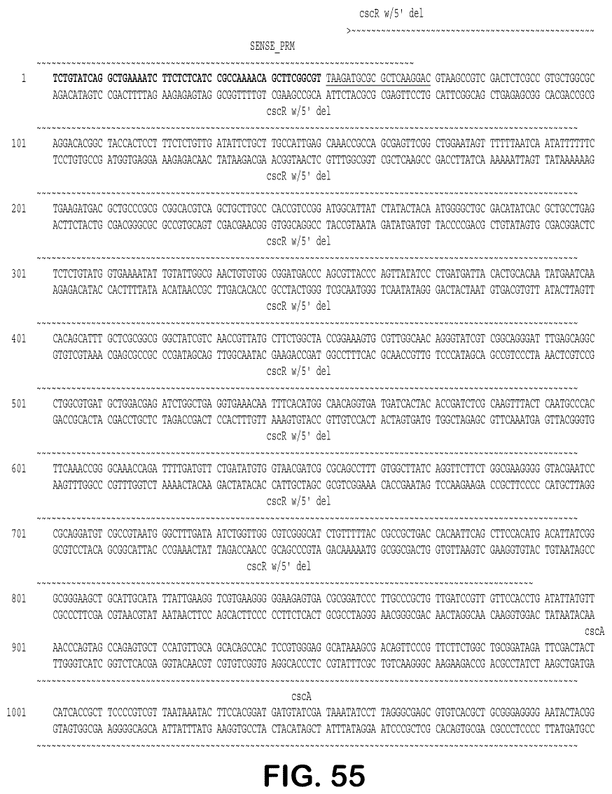

Underlined regions indicate homology to the csc operon, and bold sequence refers to sequence homology upstream and downstream of the rrnC region. The sequence of the entire PCR product is shown in FIG. 55. The PCR product was designed so that it integrated into genome into the rrnC region of the chromosome, effectively deleting 191 nucleotides upstream of rrlC (23S rRNA), all of the rrlC rRNA gene and 3 nucleotides downstream of rrlC and replacing it with the sucrose operon, as shown in FIG. 56.

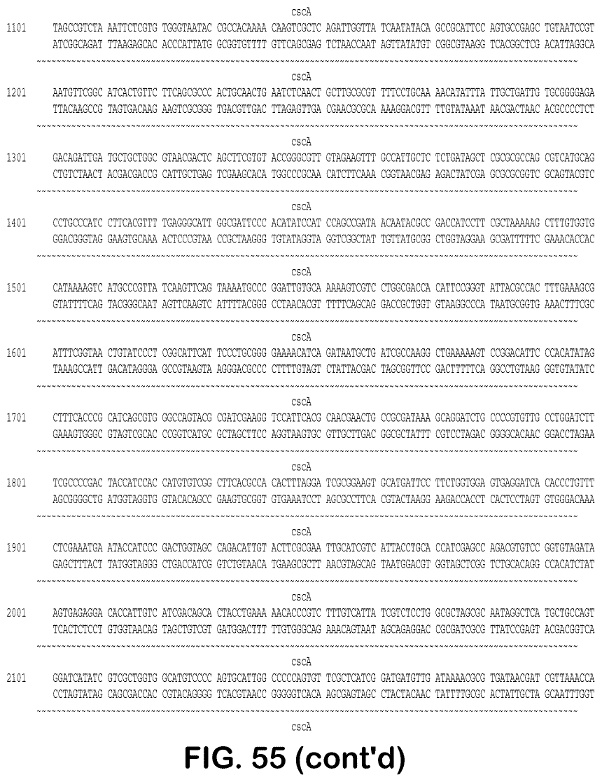

ECKh-463 was transformed with pZS*13 containing P. gingivalis Cat2 and C. beijerinckii Ald to provide a complete BDO pathway. Cells were cultured in M9 minimal medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂)) supplemented with 10 g/L sucrose. 0.2 mM IPTG was present in the culture from the start. Anaerobic conditions were maintained using a bottle with 23G needle. Asa control, ECKh-432 containing the same plasmid was cultured on the same medium, except with 10 g/L glucose instead of sucrose. FIG. 57 shows average product concentration, normalized to culture OD600, after 48 hours of growth. The data is for 6 replicate cultures of each strain. This demonstrates that BDO production from ECKh-463 on sucrose is similar to that of the parent strain on sucrose.

### Example XIX

### Summary of BDO Producing Strains

This example describes various BDO producing strains.

Table 18 summarizes various BDO producing strains disclosed above in Examples XII-XVIII.

TABLE 18
Summary of various BDO production strains.

| | Host | | | |
|---|---|---|---|---|
| Strain | Strain | | | |
| # | # | Host chromosome | Host Description | Plasmid-based |
| 1 | | ΔldhA | Single deletion | E. coli sucCD, P. gingivalis |
| | | | derivative of E. | sucD, P. gingivalis 4hbd, P. |
| | | | coli MG1655 | gingivalis Cat2, C. |
| | | | | acetobutylicum AdhE2 |
| 2 | AB3 | ΔadhE ΔldhA Δpf1B | Succinate | E. coli sucCD, P. gingivalis |
| | | | producing strain; | sucD, P. gingivalis 4hbd, P. |
| | | | derivative of E. | gingivalis Cat2, C. |
| | | | coli MG1655 | acetobutylicum AdhE2 |
| 3 | ECKh- | ΔadhE ΔldhA Δpf1B | Improvement of | E. coli sucCD, P. gingivalis |
| | 138 | ΔlpdA::K.p.lpdA322 | lpdA to increase | sucD, P. gingivalis 4hbd, P. |
| | | | pyruvate | gingivalis Cat2, C. |
| | | | dehydrogenase | acetobutylicum AdhE2 |
| | | | flux | |
| 4 | ECKh- | ΔadhE ΔldhA Δpf1B | | E. coli sucCD, P. gingivalis |
| | 138 | ΔlpdA::K.p.lpdA322 | | sucD, P. gingivalis 4hbd, C. |
| | | | | acetobutylicum buk1, C. |
| | | | | acetobutylicum ptb, C. |
| | | | | acetobutylicum AdhE2 |
| 5 | ECKh- | ΔadhE ΔldhA Δpf1B | Deletions in mdh | E. coli sucCD, P. gingivalis |
| | 401 | ΔlpdA::K.p.lpdA322 Δmdh | and arcA to direct | sucD, P. gingivalis 4hbd, P. |
| | | ΔarcA | flux through | gingivalis Cat2, C. |
| | | | oxidative TCA | acetobutylicum AdhE2 |
| | | | cycle | |
| 6 | ECKh- | ΔadhE ΔldhA ΔpflB | | M. bovis sucA, E. coli sucCD, |
| | 401 | ΔlpdA::K.p.lpdA322 Δmdh | | P. gingivalis sucD, P. |
| | | ΔarcA | | gingivalis 4hbd, P. gingivalis |
| | | | | Cat2, C. acetobutylicum |
| | | | | AdhE2 |
| 7 | ECKh- | ΔadhE ΔldhA Δpf1B | Mutation in citrate | E. coli sucCD, P. gingivalis |
| | 422 | ΔlpdA::K.p.lpdA322 Δmdh | synthase to | sucD, P. gingivalis 4hbd, P. |
| | | ΔarcA gltAR163L | improve anaerobic | gingivalis Cat2, C. |
| | | | activity | acetobutylicum AdhE2 |
| 8 | ECKh- | ΔadhE ΔldhA Δpf1B | | M. bovis sucA, E. coli sucCD, |
| | 422 | ΔlpdA::K.p.lpdA322 Δmdh | | P. gingivalis sucD, P. |
| | | ΔarcA gltAR163L | | gingivalis 4hbd, P. gingivalis |
| | | | | Cat2, C. acetobutylicum |
| | | | | AdhE2 |
| 9 | ECKh- | ΔadhE ΔldhA Δpf1B | | M. bovis sucA, E. coli sucCD, |
| | 422 | ΔlpdA::K.p.lpdA322 Δmdh | | P. gingivalis sucD, P. |
| | | ΔarcA gltAR163L | | gingivalis 4hbd, P. gingivalis |
| | | | | Cat2, C. beijerinckii Ald |
| 10 | ECKh- | ΔadhE ΔldhA Δpf1B | Succinate branch | P. gingivalis Cat2, C. |
| | 426 | ΔlpdA::K.p.lpdA322 Δmdh | of upstream | beijerinckii Ald |
| | | ΔarcA gltAR163L fimD:: E. coli | pathway integrated | |
| | | sucCD, P. gingivalis sucD, P. | into ECKh-422 | |
| | | gingivalis 4hbd | | |
| 11 | ECKh- | ΔadhE ΔldhA Δpf1B | Succinate and | P. gingivalis Cat2, C. |
| | 432 | ΔlpdA::K.p.lpdA322 Δmdh | alpha-ketoglutarate | beijerinckii Ald |
| | | ΔarcA gltAR163L fimD:: E. coli | upstream pathway | |
| | | sucCD, P. gingivalis sucD, P. | branches | |
| | | gingivalis 4hbd fimD:: M. bovis | integrated into | |
| | | sucA, C. kluyveri 4hbd | ECKh-422 | |
| 12 | ECKh- | ΔadhE ΔldhA Δpf1B | | C. acetobutylicum buk1, C. |
| | 432 | ΔlpdA::K.p.lpdA322 Δmdh | | acetobutylicum ptb, C. |
| | | ΔarcA gltAR163L fimD:: E. coli | | beijerinckii Ald |
| | | sucCD, P. gingivalis sucD, P. | | |
| | | gingivalis 4hbd fimD:: M. bovis | | |
| | | sucA, C. kluyveri 4hbd | | |
| 13 | ECKh- | ΔadhE ΔldhA Δpf1B | Acetate kinase | P. gingivalis Cat2, C. |
| | 439 | ΔlpdA::K.p.lpdA322 Δmdh | deletion of ECKh- | beijerinckii Ald |
| | | ΔarcA gltAR163L ΔackA fimD:: | 432 | |
| | | E. coli sucCD, P. gingivalis | | |
| | | sucD, P. gingivalis 4hbd fimD:: | | |
| | | M. bovis sucA, C. kluyveri 4hbd | | |
| 14 | ECKh- | ΔadhE ΔldhA Δpf1B | Acetate kinase | P. gingivalis Cat2, C. |
| | 453 | ΔlpdA::K.p.lpdA322 Δmdh | deletion and | beijerinckii Ald |
| | | ΔarcA gltAR163L ΔackA | PPC/PEPCK | |
| | | Δppc::H.i.ppck fimD:: E. coli | replacement of | |
| | | sucCD, P. gingivalis sucD, P. | ECKh-432 | |
| | | gingivalis 4hbd fimD:: M. bovis | | |
| | | sucA, C. kluyveri 4hbd | | |
| 15 | ECKh- | ΔadhE ΔldhA Δpf1B ΔlpdA:fnr- | Replacement of | P. gingivalis Cat2, C. |
| | 456 | pf1B6-K.p.lpdA322 Δmdh ΔarcA | lpdA promoter | beijerinckii Ald |
| | | gltAR163L fimD:: E. coli | with anaerobic | |
| | | sucCD, P. gingivalis sucD, P. | promoter in | |
| | | gingivalis 4hbd fimD:: M. bovis | ECKh-432 | |
| | | sucA, C. kluyveri 4hbd | | |
| 16 | ECKh- | ΔadhE ΔldhA Δpf1B ΔlpdA:: | Replacement of | P. gingivalis Cat2, C. |
| | 455 | K.p.lpdA322 ΔpdhR:: fnr-pf1B6 | pdhR and aceEF | beijerinckii Ald |
| | | Δmdh ΔarcA gltAR163L fimD:: | promoter with | |
| | | E. coli sucCD, P. gingivalis | anaerobic | |
| | | sucD, P. gingivalis 4hbd fimD:: | promoter in | |
| | | M. bovis sucA, C. kluyveri 4hbd | ECKh-432 | |
| 17 | ECKh- | ΔadhE ΔldhA ΔpflB ΔlpdA:: | Integration of | C. beijerinckii Ald |
| | 459 | K.p.lpdA322 Δmdh ΔarcA | BK/PTB into | |
| | | gltAR163L fimD:: E. coli | ECKh-432 | |
| | | sucCD, P. gingivalis sucD, P. | | |
| | | gingivalis 4hbd fimD:: M. bovis | | |
| | | sucA, C. kluyveri 4hbd fimD:: | | |
| | | C. acetobutylicum buk1, | | |
| | | C. acetobutylicum ptb | | |
| 18 | ECKh- | ΔadhE ΔldhA Δpf1B ΔlpdA:: | | C. beijerinckii Ald, G. |
| | 459 | K.p.lpdA322 Δmdh ΔarcA | | thermoglucosidasius adh1 |
| | | gltAR163L fimD:: E. coli | | |
| | | sucCD, P. gingivalis sucD, P. | | |
| | | gingivalis 4hbd fimD:: M. bovis | | |
| | | sucA, C. kluyveri 4hbd fimD:: | | |
| | | C. acetobutylicum bukl, | | |
| | | C. acetobutylicum ptb | | |
| 19 | ECKh- | ΔadhE ΔldhA Δpf1B | Non-PTS sucrose | P. gingivalis Cat2, C. |
| | 463 | ΔlpdA::K.p.lpdA322 Δmdh | genes inserted into | beijerinckii Ald |
| | | ΔarcA gltAR163L fimD:: E. coli | ECKh-432 | |
| | | sucCD, P. gingivalis sucD, P. | | |
| | | gingivalis 4hbd fimD:: M. bovis | | |
| | | sucA, C. kluyveri 4hbd | | |
| | | rrnC::cscAKB | | |
| 20 | ECKh- | ΔadhE ΔldhA Δpf1B | | C. acetobutylicum buk1, C. |
| | 463 | ΔlpdA::K.p.lpdA322 Δmdh | | acetobutylicum ptb, C. |
| | | ΔarcA gltAR163L fimD:: E. coli | | beijerinckii Ald |
| | | sucCD, P. gingivalis sucD, P. | | |
| | | gingivalis 4hbd fimD:: M. bovis | | |
| | | sucA, C. kluyveri 4hbd | | |
| | | rrnC::cscAKB | | |

The strains summarized in Table 18 are as follows. Strain 1: Single deletion derivative of E. coli MG1655, with deletion of endogenous I/AA; plasmid expression of E. coli sucCD, P. gingivalis sucD, P. gingivalis 4hbd P. gingivalis Cat2, C. acetobutylicum AdhE2. Strain 2: Host strain AB3, a succinate producing strain, derivative of E. coli MG1655, with deletions of endogenous adhE ldhA pflB; plasmid expression of E. coli sucCD, P. gingivalis sucD, P. gingivalis 4hbd P. gingivalis Cat2, C. acetobutylicum AdhE2.

Strain 3: Host strain ECKh-138, deletion of endogenous adhE, IKA, pflB, deletion of endogenous lpdA and chromosomal insertion of Klebsiella pneumoniae lpdA with a Glu354Lys mutation at the lpdA locus; plasmid expression of E. coli sucCD, P. gingivalis sucD, P. gingivalis 4hbd P. gingivalis Cat2, C. acetobutylicum AdhE2; strain provides improvement of lpdA to increase pyruvate dehydrogenase flux. Strain 4: Host strain ECKh-138, deletion of endogenous adhE, ldhA, pflB, and lpdA, chromosomal insertion of Klebsiella pneumoniae lpdA with a Glu354Lys mutation; plasmid expression E. coli sucCD, P. gingivalis sucD, P. gingivalis 4hbd C. acetobutylicum buk1, C. acetobutylicum ptb, C. acetobutylicum AdhE2.

Strain 5: Host strain ECKh-401, deletion of endogenous adhE, ldhA, pflB, deletion of endogenous lpdA and chromosomal insertion of Klebsiella pneumoniae lpdA with a Glu354Lys mutation at the lpdA locus, deletion of endogenous md and arcA; plasmid expression of E. coli sucCD, P. gingivalis sucD, P. gingivalis 4hbd P. gingivalis Cat2, C. acetobutylicum AdhE2; strain has deletions in md and arcA to direct flux through oxidative TCA cycle. Strain 6: host strain ECKh-401, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdh and arcA; plasmid expression of M. bovis sucA, E. coli sucCD, P. gingivalis sucD, P. gingivalis 4hbd P. gingivalis Cat2, C. acetobutylicum AdhE2.

Strain 7: Host strain ECKh-422, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdh and arcA, chromosomal replacement of gltA with gltA Arg163Leu mutant; plasmid expression of E. coli sucCD, P. gingivalis sucD, P. gingivalis 4hbd P. gingivalis Cat2, C. acetobutylicum AdhE2; strain has mutation in citrate synthase to improve anaerobic activity. Strain 8: strain ECKh-422, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdh and arcA, chromosomal replacement of gltA with gltA Arg163Leu mutant; plasmid expression of M. bovis sucA, E. coli sucCD, P. gingivalis sucD, P. gingivalis 4hbd P. gingivalis Cat2, C. acetobutylicum AdhE2. Strain 9: host strain ECKh-422, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdh and arcA, chromosomal replacement of gltA with gltA Arg163Leu mutant; plasmid expression of M. bovis sucA, E. coli sucCD, P. gingivalis sucD, P. gingivalis 4hbd, P. gingivalis Cat2, C. beijerinckii Ald.

Strain 10: host strain ECKh-426, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdi and arcA, chromosomal replacement of gltA with gltA Arg163Leu mutant, chromosomal insertion at the fimD locus of E. coli sucCD, P. gingivalis sucD, P. gingivalis 4hbd, plasmid expression of P. gingivalis Cat2, C. beijerinckii Ald; strain has succinate branch of upstream pathway integrated into strain ECKh-422 at the fimD locus. Strain 11: host strain ECKh-432, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdh and arcA, chromosomal replacement of gltA with gltA Arg163Leu mutant, chromosomal insertion at the fimD locus of E. coli sucCD, P. gingivalis sucD, P. gingivalis 4hbd, chromosomal insertion at the fimD locus of M. bovis sucA, C. kluyveri 4hbd, plasmid expression of P. gingivalis Cat2, C. beijerinckii Ald; strain has succinate and alpha-ketoglutarate upstream pathway branches integrated into ECKh-422. Strain 12: host strain ECKh-432, deletion of endogenous adhE, ldhA, pflB, deletion of endogenous lpdA and chromosomal insertion of Klebsiella pneumoniae lpdA with a Glu354Lys mutation at the lpdA locus, deletion of endogenous Mdh and arcA, chromosomal replacement of gltA with gltA Arg163Leu mutant, chromosomal insertion at the fimD locus of E. coli sucCD, P. gingivalis sucD, P. gingivalis 4hbd, chromosomal insertion at the fimD locus of M. bovis sucA, C. kluyveri 4hbd, plasmid expression of C. acetobutylicum buk1, C. acetobutylicum ptb, C. beijerinckii Ald.

Strain 13: host strain ECKh-439, deletion of endogenous aheE, ldhA, pflB, deletion of endogenous lpdA and chromosomal insertion of Klebsiella pneumoniae lpdA with a Glu354Lys mutation at the lpdA locus, deletion of endogenous mdh and arcA, chromosomal replacement of gltA with gltA Arg163Leu mutant, deletion of endogenous ackA, chromosomal insertion at the fimD locus of E. coli sucCD, P. gingivalis sucD, P. gingivalis 4hbd, chromosomal insertion at the fimD locus of M. bovis sucA, C. kluyveri 4hbd, plasmid expression of P. gingivalis Cat2, C. beijerinckii Ald; strain has acetate kinase deletion in strain ECKh-432. Strain 14: host strain ECKh-453, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdh and arcA, chromosomal replacement of gltA with gltA Arg163Leu mutant, deletion of endogenous ackA, deletion of endogenous ppc and insertion of Haemophilus influenza ppck at the ppc locus, chromosomal insertion at the fimD locus of E. coli sucCD, P. gingivalis sucD, P. gingivalis 4hbd, chromosomal insertion at the fimD locus of M. bovis sucA, C. kluyveri 4hbd, plasmid expression of P. gingivalis Cat2, C. beijerinckii Ald; strain has acetate kinase deletion and PPC/PEPCK replacement in strain ECKh-432.

Strain 15: host strain ECKh-456, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdi and arcA, chromosomal replacement of gltA with gltA Arg163Leu mutant, chromosomal insertion at the fimD locus of E. coli sucCD, P. gingivalis sucD, P. gingivalis 4hbd, chromosomal insertion at the fimD locus of M. bovis sucA, C. kluyveri 4hbd replacement of lpdA promoter with fnr binding site, pflB-p6 promoter and RBS of pflB; plasmid expression of P. gingivalis Cat2, C. beijerinckii Ald; strain has replacement of lpdA promoter with anaerobic promoter in strain ECKh-432. Strain 16: host strain ECKh-455, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdh and arcA, chromosomal replacement of gltA with gltA Arg163Leu mutant, chromosomal insertion at the fimD locus of E. coli sucCD, P. gingivalis sucD, P. gingivalis 4hbd, chromosomal insertion at the fimD locus of M. bovis sucA, C. kluyveri 4hbdl, replacement of pdhR and aceEF promoter with fnr binding site, pflB-p6 promoter and RBS of pflB; plasmid expression of P. gingivalis Cat2, C. beijerinckii Ald; strain has replacement of pdhR and aceEF promoter with anaerobic promoter in ECKh-432.

Strain 17: host strain ECKh-459, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdh and arcA, chromosomal replacement of gltA with gltA Arg163Leu mutant, chromosomal insertion at the fimD locus of E. coli sucCD, P. gingivalis sucD, P. gingivalis 4hbd, chromosomal insertion at the fimD locus of M. bovis sucA, C. kluyveri 4hbd, chromosomal insertion at the fimD locus of C. acetobutylicum buk1, C. acetobutylicum ptb; plasmid expression of C. beijerinckii Ald; strain has integration of BK/PTB into strain ECKh-432. Strain 18: host strain ECKh-459, deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdh and arcA, chromosomal replacement of gltA with gltA Arg163Leu mutant, chromosomal insertion at the fimD locus of E. coli sucCD, P. gingivalis sucD, P. gingivalis 4hbd, chromosomal insertion at the fimD locus of M. bovis sucA, C. kluyveri 4hbd, chromosomal insertion at the fimD locus of C. acetobutylicum buk1, C. acetobutylicum ptb; plasmid expression of C. beijerinckii Ald, G. thermoglucosidasius adh1.

Strain 19: host strain ECKh-463, deletion of endogenous aheE, ldhA, pflB, deletion of endogenous lpdA and chromosomal insertion of Klebsiella pneumoniae lpdA with a Glu354Lys mutation at the lpdA locus, deletion of endogenous mdi and arcA, chromosomal replacement of gltA with gltA Arg163Leu mutant, chromosomal insertion at the fimD locus of E. coli sucCD, P. gingivalis sucD, P. gingivalis 4hbd, chromosomal insertion at the fimD locus of M. bovis sucA, C. kluyveri 4hbd, insertion at the rrnC locus of non-PTS sucrose operon genes sucrose permease (cscB), D-fructokinase (cscK), sucrose hydrolase (cscA), and a LacI-related sucrose-specific repressor (cscR); plasmid expression of P. gingivalis Cat2, C. beijerinckii Ald; strain has non-PTS sucrose genes inserted into strain ECKh-432. Strain 20: host strain ECKh-463 deletion of endogenous adhE, ldhA, pflB, deletion of endogenous lpdA and chromosomal insertion of Klebsiella pneumoniae lpdA with a Glu354Lys mutation at the lpdA locus, deletion of endogenous mdi and arcA, chromosomal replacement of gltA with gltA Arg163Leu mutant, chromosomal insertion at the fimD locus of E. coli sucCD, P. gingivalis sucD, P. gingivalis 4hbd, chromosomal insertion at the fimD locus of M. bovis sucA, C. kluyveri 4hbd, insertion at the rrnC locus of non-PTS sucrose operon; plasmid expression of C. acetobutylicum buk1, C. acetobutylicum ptb, C. beijerinckii Ald.

In addition to the BDO producing strains disclosed herein, including those disclosed in Table 18, it is understood that additional modifications can be incorporated that further increase production of BDO and/or decrease undesirable byproducts. For example, a BDO producing strain, or a strain of Table 18, can incorporate additional knockouts to further increase the production of BDO or decrease an undesirable byproduct. Exemplary knockouts have been described previously (see U.S. publication 2009/0047719). Such knockout strains include, but are not limited to, one ore more genes selected from ADHEr, NADH6; ADHEr, PPCK; ADHEr, SUCD4; ADHEr, ATPS4r; ADHEr, FUM; ADHEr, MDH; ADHEr, PFLi, PPCK; ADHEr, PFLi, SUCD4; ADHEr, ACKr, NADH6; ADHEr, NADH6, PFLi; ADHEr, ASPT, MDH; ADHEr, NADH6, PPCK; ADHEr, PPCK, THD2; ADHEr, ATPS4r, PPCK; ADHEr, MDH, THD2; ADHEr, FUM, PFLi; ADHEr, PPCK, SUCD4; ADHEr, GLCpts, PPCK; ADHEr, GLUDy, MDH; ADHEr, GLUDy, PPCK; ADHEr, FUM, PPCK; ADHEr, MDI, PPCK; ADHEr, FUM, GLUDy; ADHEr, FUM, HEX1; ADHEr, HEX1, PFLi; ADHEr, HEX1, THD2; ADHEr, FRD2, LDH_D, MDH; ADHEr, FRD2, LDH_D, ME2; ADHEr, MDI, PGL, THD2; ADHEr, G6PDHy, MDH, THD2; ADHEr, PFLi, PPCK, THD2; ADHEr, ACKr, AKGD, ATPS4r; ADHEr, GLCpts, PFLi, PPCK; ADHEr, ACKr, ATPS4r, SUCOAS; ADHEr, GLUDy, PFLi, PPCK; ADHEr, ME2, PFLi, SUCD4; ADHEr, GLUDy, PFLi, SUCD4; ADHEr, ATPS4r, LDH_D, SUCD4; ADHEr, FUM, HEX1, PFLi; ADHEr, MDI, NADH6, THD2; ADHEr, ATPS4r, MDH, NADH6; ADHEr, ATPS4r, FUM, NADH6; ADHEr, ASPT, MDH, NADH6; ADHEr, ASPT, MDH, THD2; ADHEr, ATPS4r, GLCpts, SUCD4; ADHEr, ATPS4r, GLUDy, MDH ADHEr, ATPS4r, MDH, PPCK; ADHEr, ATPS4r, FUM, PPCK; ADHEr, ASPT, GLCpts, MDH; ADHEr, ASPT, GLUDy, MDH; ADHEr, ME2, SUCD4, THD2; ADHEr, FUM, PPCK, THD2; ADHEr, MDH, PPCK, THD2; ADHEr, GLUDy, MDH, THD2; ADHEr, HEX1, PFLi, THD2; ADHEr, ATPS4r, G6PDHy, MDH; ADHEr, ATPS4r, MDH, PGL; ADHEr, ACKr, FRD2, LDH_D; ADHEr, ACKr, LDH_D, SUCD4; ADHEr, ATPS4r, FUM, GLUDy; ADHEr, ATPS4r, FUM, HEX1; ADHEr, ATPS4r, MDH, THD2; ADHEr, ATPS4r, FRD2, LDH_D; ADHEr, ATPS4r, MDH, PGDH; ADHEr, GLCpts, PPCK, THD2; ADHEr, GLUDy, PPCK, THD2; ADHEr, FUM, HEX1, THD2; ADHEr, ATPS4r, ME2, THD2; ADHEr, FUM, ME2, THD2; ADHEr, GLCpts, GLUDy, PPCK; ADHEr, ME2, PGL, THD2; ADHEr, G6PDHy, ME2, THD2; ADHEr, ATPS4r, FRD2, LDH_D, ME2; ADHEr, ATPS4r, FRD2, LDH_D, MDH; ADHEr, ASPT, LDH_D, MDH, PFLi; ADHEr, ATPS4r, GLCpts, NADH6, PFLi; ADHEr, ATPS4r, MDI, NADH6, PGL; ADHEr, ATPS4r, G6PDHy, MDH, NADH6; ADHEr, ACKr, FUM, GLUDy, LDH_D; ADHEr, ACKr, GLUDy, LDH_D, SUCD4; ADHEr, ATPS4r, G6PDHy, MDH, THD2; ADHEr, ATPS4r, MDH, PGL, THD2; ADHEr, ASPT, G6PDHy, MDH, PYK; ADHEr, ASPT, MDH, PGL, PYK; ADHEr, ASPT, LDH_D, MDH, SUCOAS; ADHEr, ASPT, FUM, LDH_D, MDH; ADHEr, ASPT, LDH_D, MALS, MDH; ADHEr, ASPT, ICL, LDH_D, MDH ADHEr, FRD2, GLUDy, LDH_D, PPCK; ADHEr FRD2, LDH_D, PPCK, THD2; ADHEr ACKr, ATPS4r, LDH_D, SUCD4; ADHEr, ACKr, ACS, PPC, PPCK; ADHEr, GLUDy, LDH_D, PPC, PPCK; ADHEr, LDH_D, PPC, PPCK, THD2; ADHEr, ASPT, ATPS4r, GLCpts, MDH; ADHEr, G6PDHy, MDH, NADH6, THD2; ADHEr, MDH, NADH6, PGL, THD2; ADHEr, ATPS4r, G6PDHy, GLCpts, MDH; ADHEr, ATPS4r, GLCpts, MDH, PGL; ADHEr, ACKr, LDH_D, MDH, SUCD4.

Table 19 shows the reactions of corresponding genes to be knocked out of a host organism such as E. coli. The corresponding metabolite corresponding to abbreviations in Table 19 are shown in Table 20.

TABLE 19
Corresponding genes to be knocked out to prevent a particular reaction from occurring in E. coli.

| Reaction | | Genes Encoding the Enzyme(s) |
|---|---|---|
| Abbreviation | Reaction Stoichiometry* | Catalyzing Each Reaction & |
| ACKr | [c]: ac + atp <==> actp + adp | (b3115 or b2296 or b1849) |
| ACS | [c]: ac + atp + coa --> accoa + amp + ppi | b4069 |
| ACt6 | ac[p] + h[p] <==>ac[c] + h[c] | Non-gene associated |
| ADHEr | [c]: etoh + nad <==> acald + h + nadh | (b0356 or b1478 or b1241) |
| | [c]: acald + coa + nad <==> accoa + h + nadh | (b1241 or b0351) |
| AKGD | [c]: akg + coa + nad --> co2 + nadh + succoa | (b0116 and b0726 and b0727) |
| ASNS2 | [c]: asp-L + atp + nh4 --> amp + asn-L + h + ppi | b3744 |
| ASPT | [c]: asp-L --> fum + nh4 | b4139 |
| ATPS4r | adp[c] + (4) h[p] + pi[c] <==> atp[c] + (3) h[c] + | (((b3736 and b3737 and |
| | h2o[c] | b3738) and (b3731 and b3732 |
| | | and b3733 and b3734 and |
| | | b3735)) or ((b3736 and |
| | | b3737 and b3738) and (b3731 |
| | | and b3732 and b3733 and |
| | | b3734 and b3735) and b3739)) |
| CBMK2 | [c]: atp + co2 + nh4 <==> adp + cbp + (2) h | (b0521 or b0323 or b2874) |
| EDA | [c]: 2ddg6p --> g3p + pyr | b1850 |
| ENO | [c]: 2pg <==> h2o + pep | b2779 |
| FBA | [c]: fdp <==> dhap + g3p | (b2097 or b2925 or b1773) |
| FBP | [c]: fdp + h2o --> f6p + pi | (b4232 or b3925) |
| FDH2 | for[p] + (2) h[c] + q8[c] --> c02[c] + h[p] + q8h2[c] | ((b3892 and b3893 and b3894) |
| | for[p] + (2) h[c] + mqn8[c] --> co2[c] + h[p] + | or (b1474 and b1475 and |
| | mql8[c] | b1476)) |
| FRD2 | [c]: fum + mql8 --> mqn8 + succ | (b4151 and b4152 and b4153 |
| | [c]: 2dmmql8 + fum --> 2dmmq8 + succ | and b4154) |
| FTHFD | [c]: 10fthf + h2o --> for + h + thf | b1232 |
| FUM | [c]: fum + h2o <==> mal-L | (b1612 or b4122 or b1611) |
| G5SD | [c]: glu5p + h + nadph --> glu5sa + nadp + pi | b0243 |
| G6PDHy | [c]: g6p + nadp <==> 6pgl + h + nadph | b1852 |
| GLCpts | glc-D[p] + pep[c] --> g6p[c] + pyr[c] | ((b2417 and b1101 and b2415 |
| | | and b2416) or (b1817 and |
| | | b1818 and b1819 and b2415 |
| | | and b2416) or (b2417 and |
| | | b1621 and b2415 and b2416)) |
| GLU5K | [c]: atp + glu-L --> adp + glu5p | b0242 |
| GLUDy | [c]: glu-L + h2o + nadp <==> akg + h + nadph + nh4 | b1761 |
| GLYCL | [c]: gly + nad + thf --> co2 + mlthf + nadh + nh4 | (b2904 and b2903 and b2905 |
| | | and b0116) |
| HEX1 | [c]: atp + glc-D --> adp + g6p + h | b2388 |
| ICL | [c]: icit --> glx + succ | b4015 |
| LDH_D | [c]: lac-D + nad <==> h + nadh + pyr | (b2133 or b1380) |
| MALS | [c]: accoa + glx + h2o --> coa + h + mal-L | (b4014 or b2976) |
| MPH | [c]: mal-L + nad <==> h + nadh + oaa | b3236 |
| ME2 | [c]: mal-L + nadp --> co2 + nadph + pyr | b2463 |
| MTHFC | [c]: h2o + methf <==> 10fthf + h | b0529 |
| NADH12 | [c]: h + mqn8 + nadh --> mql8 + nad | b1109 |
| | [c]: h + nadh + q8 --> nad + q8h2 | |
| | [c]: 2dmmq8 + h + nadh --> 2dmmql8 + nad | |
| NADH6 | (4) h[c] + nadh[c] + q8[c] --> (3) h[p] + nad[c] + | (b2276 and b2277 and b2278 |
| | q8h2[c] | and b2279 and b2280 and |
| | (4) h[c] + mqn8[c] + nadh[c] --> (3) h[p] + mql8[c] + | b2281 and b2282 and b2283 |
| | nad[c] | and b2284 and b2285 and |
| | 2dmmq8[c] + (4) h[c] + nadh[c] --> 2dmmql8[c] + (3) | b2286 and b2287 and b2288) |
| | h[p] +nad[c] | |
| PFK | [c]: atp + f6p --> adp + fdp + h | (b3916 or b1723) |
| PFLi | [c]: coa + pyr --> accoa + for | (((b0902 and b0903) and |
| | | b2579) or (b0902 and b0903) |
| | | or (b0902 and b3114) or |
| | | (b3951 and b3952)) |
| PGDH | [c]: 6pgc + nadp --> co2 + nadph + ru5p-D | b2029 |
| PGI | [c]: g6p <==> f6p | b4025 |
| PGL | [c]: 6pgl + h2o --> 6pgc + h | b0767 |
| PGM | [c]: 2pg <==> 3pg | (b3612 or b4395 or b0755) |
| PPC | [c]: co2 + h2o + pep --> h + oaa + pi | b3956 |
| PPCK | [c]: atp + oaa --> adp + co2 + pep | b3403 |
| PRO1z | [c]: fad + pro-L --> 1pyr5c + fadh2 + h | b1014 |
| PYK | [c]: adp + h + pep --> atp + pyr | b1854 or b1676) |
| PYRt2 | h[p] + pyr[p] <==> h[c] + pyr[c] | Non-gene associated |
| RPE | [c]: ru5p-D <==> xu5p-D | (b4301 or b3386) |
| SO4t2 | so4[e] <==> so4[p] | (b0241 or b0929 or b1377 or |
| | | b2215) |
| SUCD4 | [c]: q8 + succ --> fum + q8h2 | (b0721 and b0722 and b0723 |
| | | and b0724) |
| SUCOAS | [c]: atp + coa + succ <==> adp + pi + succoa | (b0728 and b0729) |
| SULabc | atp[c] + h2o[c] + so4[p] --> adp[c] + h[c] + pi[c] + | ((b2422 and b2425 and b2424 |
| | so4[c] | and b2423) or (b0763 and |
| | | b0764 and b0765) or (b2422 |
| | | and b2424 and b2423 and |
| | | b3917)) |
| TAL | [c]: g3p + s7p <==> e4p + f6p | (b2464 or b0008) |
| THD2 | (2) h[p] + nadh[c] + nadp[c] --> (2) h[c] + nad[c] + | (b1602 and b1603) |
| | nadph[c] | |
| THD5 | [c]: nad + nadph --> nadh + nadp | (b3962 or (b1602 and b1603)) |
| TPI | [c]: dhap <==> g3p | b3919 |

TABLE 20
Metabolite names corresponding to abbreviations used in Table 19.

| Metabolite | |
|---|---|
| Abbreviation | Metabolite Name |
| 10fthf | 10-Formyltetrahy drofolate |
| 1pyr5c | 1-Pyrroline-5-carboxylate |
| 2ddg6p | 2-Dehydro-3-deoxy-D-gluconate 6-phosphate |
| 2dmmq8 | 2-Demethylmenaquinone 8 |
| 2dmmql8 | 2-Demethylmenaquinol 8 |
| 2pg | D-Glycerate 2-phosphate |
| 3pg | 3-Phospho-D-glycerate |
| 6pgc | 6-Phospho-D-gluconate |
| 6pgl | 6-phospho-D-glucono-1,5-lactone |
| ac | Acetate |
| acald | Acetaldehyde |
| accoa | Acetyl-CoA |
| actp | Acetyl phosphate |
| adp | ADP |
| akg | 2-Oxoglutarate |
| amp | AMP |
| asn-L | L-Asparagine |
| asp-L | L-Aspartate |
| atp | ATP |
| cbp | Carbamoyl phosphate |
| co2 | CO2 |
| coa | Coenzyme A |
| dhap | Dihydroxyacetone phosphate |
| e4p | D-Erythrose 4-phosphate |
| etoh | Ethanol |
| f6p | D-Fructose 6-phosphate |
| fad | Flavin adenine dinucleotide oxidized |
| fadh2 | Flavin adenine dinucleotide reduced |
| fdp | D-Fructose 1,6-bisphosphate |
| for | Formate |
| fum | Fumarate |
| g3p | Glyceraldehyde 3-phosphate |
| g6p | D-Glucose 6-phosphate |
| glc-D | D-Glucose |
| glu5p | L-Glutamate 5-phosphate |
| glu5sa | L-Glutamate 5-semialdehyde |
| glu-L | L-Glutamate |
| glx | Glyoxylate |
| gly | Glycine |
| h | H+ |
| h2o | H2O |
| icit | Isocitrate |
| lac-D | D-Lactate |
| mal-L | L-Malate |
| methf | 5,10-Methenyltetrahydrofolate |
| mlthf | 5,10-Methylenetetrahydrofolate |
| mql8 | Menaquinol 8 |
| mqn8 | Menaquinone 8 |
| nad | Nicotinamide adenine dinucleotide |
| nadh | Nicotinamide adenine dinucleotide-reduced |
| nadp | Nicotinamide adenine dinucleotide phosphate |
| nadph | Nicotinamide adenine dinucleotide phosphate-reduced |
| nh4 | Ammonium |
| oaa | Oxaloacetate |
| pep | Phosphoenolpyruvate |
| pi | Phosphate |
| ppi | Diphosphate |
| pro-L | L-Proline |
| pyr | Pyruvate |
| q8 | Ubiquinone-8 |
| q8h2 | Ubiquinol-8 |
| ru5p-D | D-Ribulose 5-phosphate |
| s7p | Sedoheptulose 7-phosphate |
| so4 | Sulfate |
| succ | Succinate |
| succoa | Succinyl-CoA |
| thf | 5,6,7,8-Tetrahydrofolate |
| xu5p-D | D-Xylulose 5-phosphate |

### Example XX

### Exemplary Pathways for Producing BDO

This example describes exemplary pathways to produce 4-hydroxybutanal (4-HBal) and/or BDO using a carboxylic acid reductase as a BDO pathway enzyme. See corresponding example in WO2013/186402. 4-Hydroxybutyrate can be derived from the tricarboxylic acid cycle intermediates succinyl-CoA and/or alpha-ketoglutarate as shown in FIG. 58.

### Example XXI

### Biosynthesis of 1,4-Butanediol Using A Carboxylic Acid Reductase Enzyme

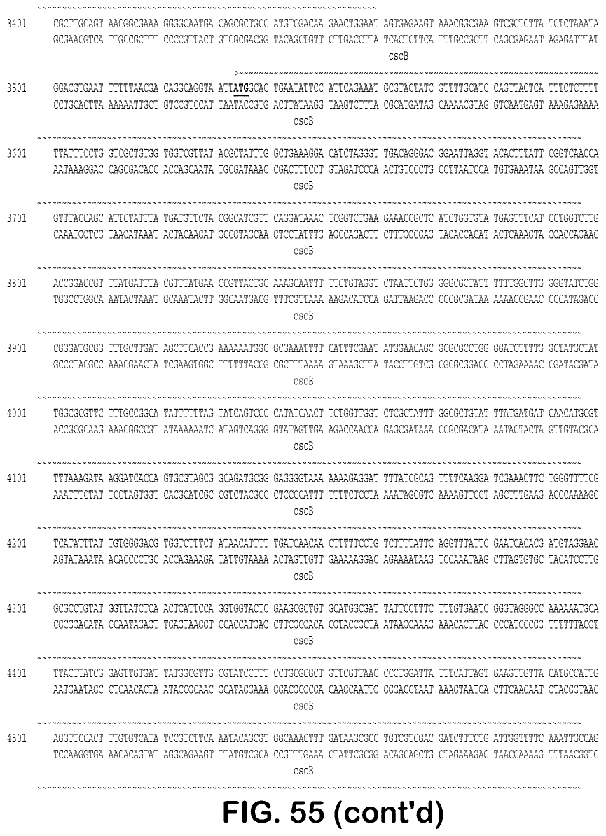

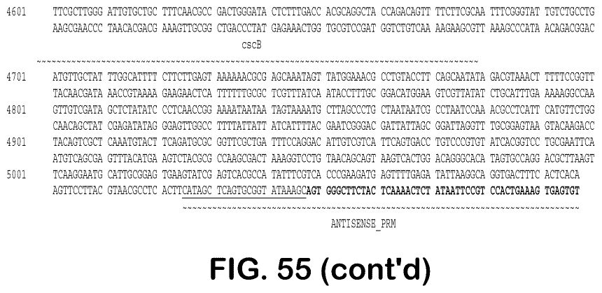

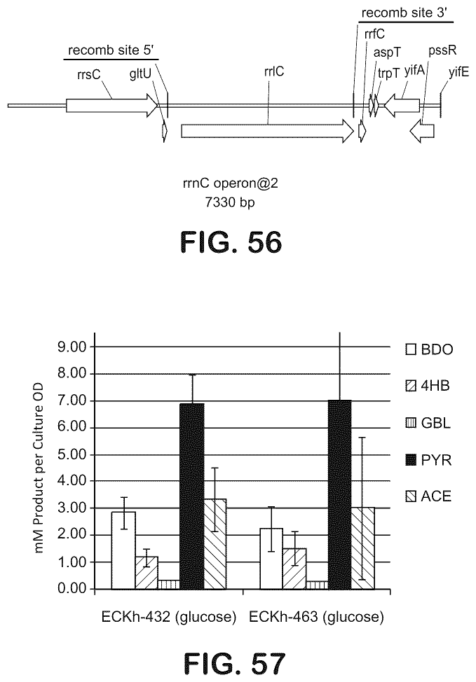

This example describes the generation of amicrobial organism that produces 1,4-butanediol using a carboxylic acid reductase enzyme. See corresponding example in WO2013/186402. The car gene (GNM_720) was cloned by PCR from Nocardia genomic DNA. Its nucleic acid and protein sequences are shown in FIGS. 59A and 59B, respectively. A codon-optimized version of the npt gene (GNM_721) was synthesized. Its nucleic acid and protein sequences are shown in FIGS. 60A and 60B, respectively. The resulting vector from cloning GNM_720 and GNM_721 into pZS*13 is shown in FIG. 61. Additional pathways to BDO employing carboxylic acid reductase (CAR) are disclosed in FIG. 62.

### Example XXII

### Pathways to Putrescine that Employ Carboxylic Acid Reductase

This example describes exemplary putrescine pathways utilizing carboxylic acid reductase. See corresponding example in WO2013/186402. FIG. 63 describes a number of additional biosynthetic pathways leading to the production of putrescine from succinate, succinyl-CoA, or alpha-ketoglutarate and employing a carboxylic acid reductase.

### Example XXIII

### Exemplary Enzymes for Production of C4 Compounds

This example describes exemplary enzymes for production of C4 compounds such as 1,4-butanediol, 4-hydroxybutanal and putrescine. See corresponding example in WO2013/186402.

Enzyme classes. All transformations depicted in FIGS. 58, 62 and 63 fall into the general categories of transformations shown in Table 21. This example describes a number of biochemically characterized genes in each category. Specifically listed are genes that can be applied to catalyze the appropriate transformations in FIGS. 58, 62 and 63 when cloned and expressed. The first three digits of each label correspond to the first three Enzyme Commission number digits which denote the general type of transformation independent of substrate specificity.

TABLE 21
Classes of Enzyme Transformations Depicted in
FIGS. 58, 62 and 63.

| LABEL | FUNCTION |
|---|---|
| 1.1.1.a | Oxidoreductase (oxo to alcohol) |
| 1.1.1.c | Oxidoreductase (2 step, acyl-CoA to alcohol) |
| 1.2.1.b | Oxidoreductase (acyl-CoA to aldehyde) |
| 1.2.1.c | Oxidoreductase (2-oxo acid to acyl-CoA, |
| | decarboxylation) |
| 1.2.1.d | Oxidoreductase (phosphonate reductase) |
| 1.2.1.e | Acid reductase |
| 1.4.1.a | Oxidoreductase (aminating) |
| 2.3.1.a | Acyltransferase (transferring phosphate group to CoA) |
| 2.6.1.a | Aminotransferase |
| 2.7.2.a | Phosphotransferase (carboxy acceptor) |
| 2.8.3.a | CoA transferase |
| 3.1.2.a | CoA hydrolase |
| 4.1.1.a | Carboxy-lyase |
| 6.2.1.a | CoA synthetase |

### 1.1.1.a Oxidoreductase (Oxo to Alcohol)

Aldehyde to alcohol. Three transformations described in FIGS. 58, 62 and 63 involve the conversion of an aldehyde to alcohol. These are 4-hydroxybutyrate dehydrogenase (step C, FIGS. 58 and 62), 1,4-butanediol dehydrogenase (step E, FIGS. 58 and 62), and 5-hydroxy-2-pentanoic acid (step Y, FIG. 62). Exemplary genes encoding enzymes that catalyze the conversion of an aldehyde to alcohol, that is, alcohol dehydrogenase or equivalently aldehyde reductase, include those recited above in EXAMPLE IV Exemplary BDO Pathways.

Enzymes exhibiting 4-hydroxybutyrate dehydrogenase activity (EC 1.1.1.61) also fall into this category. The description and enzymes for this example can be found in the above EXAMPLE IV Exemplary BDO Pathways.

The adh1 gene from Geobacillus thermoglucosidasius M10EXG (Jeon et al., J. Biotechnol. 135:127-133 (2008)) was shown to exhibit high activity on both 4-hydroxybutanal and butanal (see above). Thus this enzyme exhibits 1,4-butanediol dehydrogenase activity.

| Gene | Accession No. | GI No. | Organism |
|---|---|---|---|
| adh1 | AAR91477.1 | 40795502 | Geobacillus thermoglucosidasius |
| | | | M10EXG |

Another exemplary enzyme is 3-hydroxyisobutyrate dehydrogenase, which catalyzes the reversible oxidation of 3-hydroxyisobutyrate to methylmalonate semialdehyde. The description and enzymes for this example can be found in the above EXAMPLE IV Exemplary BDO Pathways. Several 3-hydroxyisobutyrate dehydrogenase enzymes have also been shown to convert malonic semialdehyde to 3-hydroxypropionic acid (3-HP). The description and enzymes for this example can be found in the above EXAMPLE IV Exemplary BDO Pathways.

The conversion of malonic semialdehyde to 3-HP can also be accomplished by two other enzymes, NADH-dependent 3-hydroxypropionate dehydrogenase and NADPH-dependent malonate semialdehyde reductase. The description and enzymes for this example can be found in the above EXAMPLE IV Exemplary BDO Pathways.

### 1.1.1.c Oxidoreductase (2 Step, Acyl-CoA to Alcohol).

Steps S and W of FIG. 62 depict bifunctional reductase enzymes that can form 4-hydroxybutyrate and 1,4-butanediol, respectively. Exemplary 2-step oxidoreductases that convert an acyl-CoA to alcohol include those that transform substrates such as acetyl-CoA to ethanol (for example, adhE from E. coli (Kessler et al., FEBS. Lett. 281:59-63 (1991)) and butyryl-CoA to butanol (for example, ad2E2 from C. acetobutylicum (Fontaine et al., J. Bacteriol. 184:821-830 (2002)). The C. acetobutylicum adE2 gene was shown to convert 4-hydroxybutyryl-CoA to 1,4-butanediol (see above). In addition to reducing acetyl-CoA to ethanol, the enzyme encoded by adhE in Leuconostoc mesenteroides has been shown to oxidize the branched chain compound isobutyraldehyde to isobutyryl-CoA (Kazahaya et al. J., Gen. Appl. Microbiol. 18:43-55 (1972); Koo et al., Biotechnol. Lett. 27:505-510 (2005)).

| Gene | Accession No. | GI No. | Organism |
|---|---|---|---|
| adhE | NP_415757.1 | 16129202 | Escherichia coli |
| adhE2 | AAK09379.1 | 12958626 | Clostridium acetobutylicum |
| adhE | AAV66076.1 | 55818563 | Leuconostoc mesenteroides |

Another exemplary enzyme can convert malonyl-CoA to 3-HP The description and enzymes for this example can be found in the above Example IV Exemplary BDO Pathways.

Longer chain acyl-CoA molecules can be reduced by enzymes such as the jojoba (Simmondsia chinensis) FAR, which encodes an alcohol-forming fatty acyl-CoA reductase. Its overexpression in E. coli resulted in FAR activity and the accumulation of fatty alcohol (Metz et al., Plant Physiol. 122:635-644 2000)).

| Gene | Accession No. | GI No. | Organism |
|---|---|---|---|
| FAR | AAD38039.1 | 5020215 | Simmondsia chinensis |

### 1.2.1.b Oxidoreductase (Acyl-CoA to Aldehyde).

Step A of FIGS. 58, 62 and 63 involves the conversion of succinyl-CoA to succinate semialdehyde by an aldehyde forming succinyl-CoA reductase. Step Q of FIG. 62 depicts the conversion of 4-hydroxybutyryl-CoA to 4-hydroxybutanal by an aldehyde-forming 4-hydroxybutyryl-CoA reductase. A description and enzymes of this class can be found in above Example IV Exemplary BDO Pathways. In addition to reducing acetyl-CoA to ethanol, the enzyme encoded by ad2E in Leuconostoc mesenteroides has been shown to oxidize the branched chain compound isobutyraldehyde to isobutyryl-CoA (Koo et al., Biotechnol. Lett. 27:505-510 (2005)). Butyraldehyde dehydrogenase catalyzes a similar reaction, conversion of butyryl-CoA to butyraldehyde, in solventogenic organisms such as Clostridium saccharoperbutylacetonicum (Kosaka et al., Biosci. Biotechnol. Biochem. 71:58-68 (2007)).

| Gene | Accession No. | GI No. | Organism |
|---|---|---|---|
| acr1 | YP_047869.1 | 50086359 | Acinetobacter calcoaceticus |
| acr1 | AAC45217 | 1684886 | Acinetobacter baylyi |
| acr1 | BAB85476.1 | 18857901 | Acinetobacter sp. Strain M-1 |
| sucD | P38947.1 | 730847 | Clostridium kluyveri |
| sucD | NP_904963.1 | 34540484 | Porphyromonas gingivalis |
| bphG | BAA03892.1 | 425213 | Pseudomonas sp |
| adhE | AAV66076.1 | 55818563 | Leuconostoc mesenteroides |
| bld | AAP42563.1 | 31075383 | Clostridium |
| | | | saccharoperbutylacetonicum |

An additional enzyme type that converts an acyl-CoA to its corresponding aldehyde is malonyl-CoA reductase, which transforms malonyl-CoA to malonic semialdehyde. A description and enzymes of this class can be found in above Example IV Exemplary BDO Pathways. Yet another candidate for CoA-acylating aldehyde dehydrogenase is the ald gene from Clostridium beijerinckii (Toth et al., Appl. Environ. Microbiol. 65:4973-4980 (1999)). This enzyme has been reported to reduce acetyl-CoA and butyryl-CoA to their corresponding aldehydes. This gene is very similar to eutE that encodes acetaldehyde dehydrogenase of Salmonella typhimurium and E. coli (Toth et al., Appl. Environ. Microbiol. 65:4973-4980 (1999)).

| Gene | Accession No. | GI No. | Organism |
|---|---|---|---|
| Msed_0709 | YP_001190808.1 | 146303492 | Metallosphaera sedula |
| mcr | NP_378167.1 | 15922498 | Sulfolobus tokodaii |
| asd-2 | NP_343563.1 | 15898958 | Sulfolobus |
| | | | solfataricus |
| Saci_2370 | YP_256941.1 | 70608071 | Sulfolobus |
| | | | acidocaldarius |
| Ald | AAT66436 | 49473535 | Clostridium |
| | | | beijerinckii |
| eutE | AAA80209 | 687645 | Salmonella |
| | | | typhimurium |
| eutE | P77445 | 2498347 | Escherichia coli |

### 1.2.1.c Oxidoreductase (2-Oxo Acid to Acyl-CoA, Decarboxylation).

Step AA in FIG. 62 depicts the conversion of 5-hydroxy-2-oxopentanoic acid to 4-hydroxybutyryl-CoA. Candidate enzymes for this transformation include 1) branched-chain 2-keto-acid dehydrogenase, 2) alpha-ketoglutarate dehydrogenase, and 3) the pyruvate dehydrogenase multienzyme complex (PDHC). A description and enzymes of this class can be found in above Example IV Exemplary BDO Pathways.

Alpha-ketoglutarate dehydrogenase (AKGD) converts alpha-ketoglutarate to succinyl-CoA and is the primary site of control of metabolic flux through the TCA cycle (Hansford, R. G. Curr. Top. Bioenerg. 10:217-278 (1980)). A description and enzymes of this class can be found in above Example IV Exemplary BDO Pathways.

Branched-chain 2-keto-acid dehydrogenase complex (BCKAD), also known as 2-oxoisovalerate dehydrogenase, participates in branched-chain amino acid degradation pathways, converting 2-keto acids derivatives of valine, leucine and isoleucine to their acyl-CoA derivatives and CO2. A description and enzymes of this class can be found in above Example IV Exemplary BDO Pathways.

The pyruvate dehydrogenase complex, catalyzing the conversion of pyruvate to acetyl-CoA, has also been extensively studied. In the E. coli enzyme, specific residues in the E1 component are responsible for substrate specificity (Bisswanger, H. J Biol Chem. 256:815-822(1981); Bremer, J. Eur. J. Biochem. 8:535-540 (1969); Gong et al. J Biol Chem. 275:13645-13653 (2000)). A description and enzymes of this class can be found in above Example IV Exemplary BDO Pathways.

As an alternative to the large multienzyme 2-keto-acid dehydrogenase complexes described above, some anaerobic organisms utilize enzymes in the 2-ketoacid oxidoreductase family (OFOR) to catalyze acylating oxidative decarboxylation of 2-keto-acids. A description and enzymes of this class can be found in above Example IV Exemplary BDO Pathways.

### 1.2.1.d Oxidoreductase (Phosphonate Reductase).

The conversion of 4-hydroxybutyryl-phosphate to 4-hydroxybutanal can be catalyzed by an oxidoreductase in the EC class 1.2.1. Aspartate semialdehyde dehydrogenase (ASD, EC 1.2.1.11) catalyzes the NADPH-dependent reduction of 4-aspartyl phosphate to aspartate-4-semialdehyde. ASD participates in amino acid biosynthesis and recently has been studied as an antimicrobial target (Hadfield et al., Biochemistry 40:14475-14483 (2001). The E. coli ASD structure has been solved (Hadfield et al., J. Mol. Biol. 289:991-1002 (1999)) and the enzyme has been shown to accept the alternate substrate beta-3-methylaspartyl phosphate (Shames et al., J. Biol. Chem. 259:15331-15339 (1984)). The Haemophilus influenzae enzyme has been the subject of enzyme engineering studies to alter substrate binding affinities at the active site (Blanco et al., Acta Crystallogr. D. Biol. Crystallogr. 60:1388-1395 (2004); Blanco et al., Acta Crystallogr. D. Biol. Crystallogr. 60:1808-1815 (2004)). Other ASD candidates are found in Mycobacterium tuberculosis (Shafiani et al., J. Appl. Microbiol. 98:832-838 (2005), Methanococcus jannaschii (Faehnle et al., J. Mol. Biol. 353:1055-1068 (2005)), and the infectious microorganisms Vibrio cholera and Heliobacter pylori (Moore et al., Protein Expr. Purif. 25:189-194 (2002)). A related enzyme candidate is acetylglutamylphosphate reductase (EC 1.2.1.38), an enzyme that naturally reduces acetylglutamylphosphate to acetylglutamate-5-semialdehyde, found in S. cerevisiae (Pauwels et al., Eur. J. Biochem. 270:1014-1024 (2003), B. subtilis (O'Reilly and Devine, Microbiology 140 (Pt 5):1023-1025 (1994)). and other organisms.

| Gene | Accession No. | GI No. | Organism |
|---|---|---|---|
| asd | NP_417891.1 | 16131307 | Escherichia coli |
| asd | YP_248335.1 | 68249223 | Haemophilus influenzae |
| asd | AAB49996 | 1899206 | Mycobacterium |
| | | | tuberculosis |
| VC2036 | NP_231670 | 15642038 | Vibrio cholera |
| asd | YP_002301787.1 | 210135348 | Heliobacter pylori |
| ARG5,6 | NP_010992.1 | 6320913 | Saccharomyces cerevisiae |
| argC | NP_389001.1 | 16078184 | Bacillus subtilis |

Other exemplary enzymes in this class include glyceraldehyde 3-phosphate dehydrogenase which converts glyceraldehyde-3-phosphate into D-glycerate 1,3-bisphosphate (for example, E. coli gapA (Branlant and Branlant,. Eur. J. Biochem. 150:61-66 (1985)), N-acetyl-gamma-glutamyl-phosphate reductase which converts N-acetyl-L-glutamate-5-semialdehyde into N-acetyl-L-glutamyl-5-phosphate (for example, E. coli argC (Parsot et al., Gene 68:275-283 (1988)), and glutamate-5-semialdehyde dehydrogenase, which converts L-glutamate-5-semialdehyde into L-glutamyl-5-phospate (for example, E. coli proA (Smith et al., J. Bacteriol. 157:545-551 (1984)). Genes encoding glutamate-5-semialdehyde dehydrogenase enzymes from Salmonella typhimurium (Mahan and Csonka, J. Bacteriol. 156:1249-1262(1983)) and Campylobacter jejuni (Louie and Chan, Mol. Gen. Genet. 240:29-35 (1993)) were cloned and expressed in E. coli.

| Gene | Accession No. | GI No. | Organism |
|---|---|---|---|
| gapA | P0A9B2.2 | 71159358 | Escherichia coli |
| argC | NP_418393.1 | 16131796 | Escherichia coli |
| proA | NP_414778.1 | 16128229 | Escherichia coli |
| proA | NP_459319.1 | 16763704 | Salmonella typhimurium |
| proA | P53000.2 | 9087222 | Campylobacter jejuni |

### 1.2.1.e Acid Reductase.

Several steps in FIGS. 58, 62 and 63 depict the conversion of unactivated acids to aldehydes by an acid reductase. These include the conversion of 4-hydroxybutyrate, succinate, alpha-ketoglutarate, and 4-aminobutyrate to 4-hydroxybutanal, succinate semialdehyde, 2,5-dioxopentanoate, and 4-aminobutanal, respectively. One notable carboxylic acid reductase can be found in Nocardia iowensis which catalyzes the magnesium, ATP and NADPH-dependent reduction of carboxylic acids to their corresponding aldehydes (Venkitasubramanian et al., J. Biol. Chem. 282:478-485 (2007)). This enzyme is encoded by the car gene and was cloned and functionally expressed in E. coli (Venkitasubramanian et al., J. Biol. Chem. 282:478485 (2007)). Expression of the npt gene product improved activity of the enzyme via post-transcriptional modification. The npt gene encodes a specific phosphopantetheine transferase (PPTase) that converts the inactive apo-enzyme to the active holo-enzyme. The natural substrate of this enzyme is vanillic acid, and the enzyme exhibits broad acceptance of aromatic and aliphatic substrates (Venkitasubramanian et al., in Biocatalysis in the Pharmaceutical and Biotechnology Industries, ed. R. N. Patel, Chapter 15, pp. 425-440, CRC Press LLC, Boca Raton, FL. (2006)).

| Gene | Accession No. | GI No. | Organism |
|---|---|---|---|
| car | AAR91681.1 | 40796035 | Nocardia iowensis |
| | | | (sp. NRRL 5646) |
| npt | ABI83656.1 | 114848891 | Nocardia iowensis |
| | | | (sp. NRRL 5646) |

Additional car and npt genes can be identified based on sequence homology.

TABLE 23

| Gene | Accession No. | GI No. | Organism |
|---|---|---|---|
| fadD9 | YP_978699.1 | 121638475 | Mycobacterium bovis |
| | | | BCG |
| BCG_2812c | YP_978898.1 | 121638674 | Mycobacterium bovis |
| | | | BCG |
| nfa20150 | YP_118225.1 | 54023983 | Nocardia farcinica |
| | | | IFM 10152 |
| nfa40540 | YP_120266.1 | 54026024 | Nocardia farcinica |
| | | | IFM 10152 |
| SGR_6790 | YP_001828302.1 | 182440583 | Streptomyces griseus |
| | | | subsp. griseus |
| | | | NBRC 13350 |
| SGR_665 | YP_001822177.1 | 182434458 | Streptomyces griseus |
| | | | subsp. griseus |
| | | | NBRC 13350 |

An additional enzyme candidate found in Streptomyces griseus is encoded by the griC and griD genes. This enzyme is believed to convert 3-amino-4-hydroxybenzoic acid to 3-amino-4-hydroxybenzaldehyde as deletion of either griC or griD led to accumulation of extracellular 3-acetylamino-4-hydroxybenzoic acid, a shunt product of 3-amino-4-hydroxybenzoic acid metabolism (Suzuki, et al., J. Antibiot. 60(6):380-387 (2007)). Co-expression of griC and griD with SGR_665, an enzyme similar in sequence to the Nocardia iowensis npt, can be beneficial.

| Gene | Accession No. | GI No. | Organism |
|---|---|---|---|
| griC | 182438036 | YP_001825755.1 | Streptomyces griseus subsp. |
| | | | griseus NBRC 13350 |
| griD | 182438037 | YP_001825756.1 | Streptomyces griseus subsp. |
| | | | griseus NBRC 13350 |
| MSMEG_2956 | YP_887275.1 | YP_887275.1 | Mycobacterium smegmatis MC2 155 |
| MSMEG_5739 | YP_889972.1 | 118469671 | Mycobacterium smegmatis MC2 155 |
| MSMEG_2648 | YP_886985.1 | 118471293 | Mycobacterium smegmatis MC2 155 |
| MAP1040c | NP_959974.1 | 41407138 | Mycobacterium avium subsp. |
| | | | paratuberculosis K-10 |
| MAP2899c | NP_961833.1 | 41408997 | Mycobacterium avium subsp. |
| | | | paratuberculosis K-10 |
| MMAR_2117 | YP_001850422.1 | 183982131 | Mycobacterium marinum M |
| MMAR_2936 | YP_001851230.1 | 183982939 | Mycobacterium marinum M |
| MMAR_1916 | YP_001850220.1 | 183981929 | Mycobacterium marinum M |
| TpauDRAFT_33060 | ZP_04027864.1 | 227980601 | Tsukamurella paurometabola DSM 20162 |
| TpauDRAFT_20920 | ZP_04026660.1 | 227979396 | Tsukamurella paurometabola DSM 20162 |
| CPCC7001_1320 | ZP_05045132.1 | 254431429 | Cyanobium PCC7001 |
| DDBDRAFT_0187729 | XP_636931.1 | 66806417 | Dictyostelium discoideum AX4 |

An enzyme with similar characteristics, alpha-aminoadipate reductase (AAR, EC 1.2.1.31), participates in lysine biosynthesis pathways in some fungal species. This enzyme naturally reduces alpha-amino adipate to alpha-aminoadipate semialdehyde. The carboxyl group is first activated through the ATP-dependent formation of an adenylate that is then reduced by NAD(P)H to yield the aldehyde and AMP Like CAR, this enzyme utilizes magnesium and requires activation by a PPTase. Enzyme candidates for AAR and its corresponding PPTase are found in Saccharomyces cerevisiae (Morris et al., Gene 98:141-145 (1991)), Candida albicans (Guo et al., Mol. Genet. Genomics 269:271-279(2003)), and Schizosaccharomyces pombe (Ford et al., Curr. Genet. 28:131-137 (1995)). The AAR from S. pombe exhibited significant activity when expressed in E. coli(Guo et al., Yeast 321:1279-1288(2004)). The AAR from Penicillium chrysogenum accepts S-carboxymethyl-L-cysteine as an alternate substrate, but did not react with adipate, L-glutamate or diaminopimelate (Hijarrubia et al., J. Biol. Chem. 278:8250-8256 (2003)). The gene encoding the P. chrysogenum PPTase has not been identified to date.

| Gene | Accession No. | GI No. | Organism |
|---|---|---|---|
| LYS2 | AAA34747.1 | 171867 | Saccharomyces cerevisiae |
| LYS5 | P50113.1 | 1708896 | Saccharomyces cerevisiae |
| LYS2 | AAC02241.1 | 2853226 | Candida albicans |
| LYS5 | AAO26020.1 | 28136195 | Candida albicans |
| Lys1p | P40976.3 | 13124791 | Schizosaccharomyces pombe |
| Lys7p | Q10474.1 | 1723561 | Schizosaccharomyces pombe |
| Lys2 | CAA74300.1 | 3282044 | Penicillium chrysogenum |

### 1.4.1.a Oxidoreductase (Aminating).

Glutamate dehydrogenase (Step J, FIGS. 62 and 63), 4-aminobutyrate dehydrogenase (Step M, FIGS. 62 and 63), putrescine dehydrogenase (Step D, FIG. 63), 5-amino-2-oxopentanoate dehydrogenase (Step P, FIG. 63), and omithine dehydrogenase (Step S, FIG. 63) can be catalyzed by aminating oxidoreductases. Enzymes in this EC class catalyze the oxidative deamination of alpha-amino acids with NAD+ or NADP+ as acceptor, and the reactions are typically reversible. Exemplary oxidoreductases operating on amino acids include glutamate dehydrogenase (deaminating), encoded by gdhA, leucine dehydrogenase (deaminating), encoded by ldh, and aspartate dehydrogenase (deaminating), encoded by nadX Description and exemplary enzymes can be found above in Example IV Exemplary BDO Pathways, section on EC 1.4.1.a—Oxidoreductase operating on amino acids.

Additional glutamate dehydrogenase gene candidates are found in Bacillus subtilis (Khan et al., Biosci. Biotechnol. Biochem. 69:1861-1870 (2005)), Nicotiana tabacum (Purnell et al., Planta 222:167-180 (2005)), Oryza sativa (Abiko et al., Plant Cell Physiol. 46:1724-1734 (2005)), Haloferax mediterranei (Diaz et al., Extremophiles 10:105-115 (2006)) and Halobacterium salinarum (Hayden et al., FEMS Microbiol. Lett. 211:3741 (2002)). The Nicotiana tabacum enzyme is composed of alpha and beta subunits encoded by gdh1 and gdh2 (Purnell et al., Planta 222:167-180 (2005)). Overexpression of the NADH-dependent glutamate dehydrogenase was found to improve ethanol production in engineered strains of S. cerevisiae (Roca et al., Appl. Environ. Microbiol. 69:4732-4736 (2003)).

| Gene | Accession No. | GI No. | Organism |
|---|---|---|---|
| rocG | NP_391659.1 | 16080831 | Bacillus subtilis |
| gdh1 | AAR11534.1 | 38146335 | Nicotiana tabacum |
| gdh2 | AAR11535.1 | 38146337 | Nicotiana tabacum |
| GDH | Q852M0 | 75243660 | Oryza sativa |
| GDH | Q977U6 | 74499858 | Haloferax mediterranei |
| GDH | P29051 | 118549 | Halobactreium salinarum |
| GDH2 | NP_010066.1 | 6319986 | Saccharomyces cerevisiae |

An exemplary enzyme for catalyzing the conversion of aldehydes to their corresponding primary amines is lysine 6-dehydrogenase (EC 1.4.1.18), encoded by the lysDH genes. The lysine 6-dehydrogenase (deaminating), encoded by lysDH gene, catalyze the oxidative deamination of the ε-amino group of L-lysine to form 2-aminoadipate-6-semialdehyde, which in turn nonenzymatically cyclizes to form Δ1-piperidine-6-carboxylate (Misono and Nagasaki, J. Bacteriol. 150:398401 (1982)). The lysDH gene from Geobacillus stearothermophilus encodes a thermophilic NAD-dependent lysine 6-dehydrogenase (Heydari et al., Appl. Environ. Microbiol 70:937-942 (2004)). The lysDH gene from Aeropyrum pernix K1 is identified through homology from genome projects. Additional enzymes can be found in Agrobacterium tumefaciens (Hashimoto et al., J. Biochem. 106:76-80 (1989); Misono and Nagasaki, J. Bacteriol. 150:398-401 (1982)) and Achromobacter denitrificans (Ruldeekulthamrong et al., BMB. Rep. 41:790-795 (2008)).

| Gene | Accession No. | GI No. | Organism |
|---|---|---|---|
| lysDH | BAB39707 | 13429872 | Geobacillus stearothermophilus |
| lysDH | NP_147035.1 | 14602185 | Aeropyrum pernix K1 |
| lysDH | NP_353966 | 15888285 | Agrobacterium tumefaciens |
| lysDH | AAZ94428 | 74026644 | Achromobacter denitrificans |

An enzyme that converts 3-oxoacids to 3-amino acids is 3,5-diaminohexanoate dehydrogenase (EC 1.4.1.11), an enzyme found in organisms that ferment lysine. The gene encoding this enzyme, kdd, was recently identified in Fusobacterium nucleatum (Kreimeyer et al., J. Biol. Chem. 282:7191-7197 (2007)). The enzyme has been purified and characterized in other organisms (Baker et al., J. Biol. Chem. 247:7724-7734 (1972); Baker and van der Drift, Biochemistry 13:292-299 (1974)), but the genes associated with these enzymes are not known. Candidates in Myxococcus xanthus, Porphyromonas gingivalis W83 and other sequenced organisms can be inferred by sequence homology.

| Gene | Accession No. | GI No. | Organism |
|---|---|---|---|
| kdd | AAL93966.1 | 19713113 | Fusobacterium nucleatum |
| mxan_4391 | ABF87267.1 | 108462082 | Myxococcus xanthus |
| pg_1069 | AAQ66183.1 | 34397119 | Porphyromonas gingivalis |

### 2.3.1.a Acyltransferase (Transferring Phosphate Group to CoA).

Step P of FIG. 62 depicts the transformation of 4-hydroxybutyryl-CoA to 4-hydroxybutyryl-Pi. Exemplary phosphate transferring acyltransferases include phosphotransacetylase, encoded by pta, and phosphotransbutyrylase, encoded by ptb. Description and exemplary enzymes can be found above in Example IV Exemplary BDO Pathways, section on EC 2.3.1.a—Acyltransferase.

### 2.6.1. Aminotransferase.

Aminotransferases reversibly convert an aldehyde or ketone to an amino group. Common amino donor/acceptor combinations include glutamate/alpha-ketoglutarate, alanine/pyruvate, and aspartate/oxaloacetate. Several enzymes have been shown to convert aldehydes to primary amines, and vice versa, such as 4-aminobutyrate, putrescine, and 5-amino-2-oxopentanoate. These enzymes are particularly well suited to carry out the following transformations: Step N in FIGS. 62 and 63, Steps E and Q in FIG. 63. Lysine-6-aminotransferase (EC 2.6.1.36) is one exemplary enzyme capable of forming a primary amine. This enzyme function, converting lysine to alpha-aminoadipate semialdehyde, has been demonstrated in yeast and bacteria. Candidates from Candida utilis (Hammer and Bode, J. Basic Microbiol. 32:21-27 (1992)), Flavobacterium lutescens (Fujii et al., J. Biochem. 128:391-397 (2000)) and Streptomyces clavuligenus (Romero et al., J. Ind. Microbiol. Biotechnol. 18:241-246 (1997)) have been characterized. A recombinant lysine-6-aminotransferase from S. clavuligenus was functionally expressed in E. coli (Tobin et al., J. Bacteriol. 173:6223-6229 (1991)). The F. lutescens enzyme is specific to alpha-ketoglutarate as the amino acceptor (Soda and Misono, Biochemistry 7:4110-4119 (1968)). Other enzymes which convert aldehydes to terminal amines include the dat gene product in Acinetobacter baumanii encoding 2,4-diaminobutanoate:2-ketoglutarate 4-transaminase (Ikai and Yamamoto, J. Bacteriol. 179:5118-5125 (1997)). In addition to its natural substrate, 2,4-diaminobutyrate, DAT transaminates the terminal amines of lysine, 4-aminobutyrate and ornithine.

| Gene | Accession No. | GI No. | Organism |
|---|---|---|---|
| lat | BAB13756.1 | 10336502 | Flavobacterium lutescens |
| lat | AAA26777.1 | 153343 | Streptomyces clavuligenus |
| dat | P56744.1 | 6685373 | Acinetobacter baumanii |

The conversion of an aldehyde to a terminal amine can also be catalyzed by gamma-aminobutyrate transaminase (GABA transaminase or 4-aminobutyrate transaminase). This enzyme naturally interconverts succinic semialdehyde and glutamate to 4-aminobutyrate and alpha-ketoglutarate and is known to have a broad substrate range (Liu et al., Biochemistry 43:10896-10905 2004); Schulz et al., Appl. Environ. Microbiol. 56:1-6 (1990)). The two GABA transaminases in E. coli are encoded by gabT (Bartsch et al., J. Bacteriol. 172:7035-7042(1990)) and puuE (Kurihara et al., J. Biol. Chem. 280:4602-4608. (2005)). GABA transaminases in Mus musculus, Pseudomonas fluorescens, and Sus scrofa have been shown to react with a range of alternate substrates including 6-aminocaproic acid (Cooper, Methods Enzymol. 113:80-82(1985); Scott and Jakoby, J. Biol. Chem. 234:932-936 (1959)).

| Gene | Accession No. | GI No. | Organism |
|---|---|---|---|
| gabT | NP_417148.1 | 16130576 | Escherichia coli |
| puuE | NP_415818.1 | 16129263 | Escherichia coli |
| abat | NP_766549.2 | 37202121 | Mus musculus |
| gabT | YP_257332.1 | 70733692 | Pseudomonas fluorescens |
| abat | NP_999428.1 | 47523600 | Sus scrofa |

Additional enzyme candidates for interconverting aldehydes and primary amines are putrescine transminases or other diamine aminotransferases. The E col putrescine aminotransferase is encoded by the ygjG gene, and the purified enzyme also was able to transaminate cadaverine and spermidine (Samsonova et al., BMC Microbiol. 3:2 (2003)). In addition, activity of this enzyme on 1,7-diaminoheptane and with amino acceptors other than 2-oxoglutarate (for example, pyruvate, 2-oxobutanoate) has been reported (Kim, J. Biol. Chem. 239:783-786 (1964); Samsonova et al., BMC Microbiol. 3:2 (2003)). A putrescine aminotransferase with higher activity with pyruvate as the amino acceptor than alpha-ketoglutarate is the spuC gene of Pseudomonas aeruginosa (Lu et al., J. Bacteriol. 184:3765-3773 (2002)).

| Gene | Accession No. | GI No. | Organism |
|---|---|---|---|
| ygjG | NP_417544 | 145698310 | Escherichia coli |
| spuC | AAG03688 | 9946143 | Pseudomonas aeruginosa |

Enzymes that transaminate 3-oxoacids include GABA amino transferase (described above), beta-alanine/alpha-ketoglutarate aminotransferase and 3-amino-2-methylpropionate aminotransferase. Beta-alanine/alpha-ketoglutarate aminotransferase (WO08027742) reacts with beta-alanine to form malonic semialdehyde, a 3-oxoacid. The gene product of SkPYD4 in Saccharomyces kluyveri was shown to preferentially use beta-alanine as the amino group donor (Andersen and Hansen, Gene 124:105-109 (1993)). SkUGA1 encodes a homologue of Saccharomyces cerevisiae GABA aminotransferase, UGA1 (Ramos et al., Eur. J. Biochem. 149:401404 (1985)), whereas SkPYD4 encodes an enzyme involved in both beta-alanine and GABA transamination (Andersen and Hansen, Gene 124:105-109 (1993)). 3-Amino-2-methylpropionate transaminase catalyzes the transformation from methylmalonate semialdehyde to 3-amino-2-methylpropionate. The enzyme has been characterized in Rattus norvegicus and Sus scrofa and is encoded by Abat (Kakimoto et al., Biochim. Biophys. Acta 156:374-380 (1968); Tamaki et al., Methods Enzymol. 324:376-389 (2000)).

| Gene | Accession No. | GI No. | Organism |
|---|---|---|---|
| SkyPYD4 | ABF58893.1 | 98626772 | Lachancea kluyveri |
| SkUGA1 | ABF58894.1 | 98626792 | Lachancea kluyveri |
| UGA1 | NP_011533.1 | 6321456 | Saccharomyces cerevisiae |
| Abat | P50554.3 | 122065191 | Rattus norvegicus |
| Abat | P80147.2 | 120968 | Sus scrofa |

Several aminotransferases transaminate the amino groups of amino acids to form 2-oxoacids. Aspartate aminotransferase is an enzyme that naturally transfers an oxo group from oxaloacetate to glutamate, forming alpha-ketoglutarate and aspartate. Aspartate is similar in structure to OHED and 2-AHD. Aspartate aminotransferase activity is catalyzed by, for example, the gene products of aspC from Escherichia coli(Yagi et al., FEBS Lett. 100:81-84 (1979); Yagi et al., Methods Enzymol. 113:83-89 (1985)), AAT2 from Saccharomyces cerevisiae (Yagi et al., J. Biochem. 92:35-43 (1982)) and ASP5 from Arabidopsis thaliana (de la Torre et al., Plant J. 46:414425 (2006); Kwok and Hanson. J. Exp. Bot. 55:595-604 (2004); Wilkie and Warren, Protein Expr. Purif. 12:381-389(1998)). The enzyme from Rattus norvegicus has been shown to transaminate alternate substrates such as 2-aminohexanedioic acid and 2,4-diaminobutyric acid (Recasens et al., Biochemistry 19:45834589 (1980)). Aminotransferases that work on other amino-acid substrates can also be able to catalyze this transformation. Valine aminotransferase catalyzes the conversion of valine and pyruvate to 2-ketoisovalerate and alanine. The E. coli gene, avtA, encodes one such enzyme (Whalen and Berg, J. Bacteriol. 150:739-746 (1982)). This gene product also catalyzes the transamination of α-ketobutyrate to generate α-aminobutyrate, although the amine donor in this reaction has not been identified (Whalen and Berg, J. Bacteriol. 158:571-574 1984)). The gene product of the E. coli serC catalyzes two reactions, phosphoserine aminotransferase and phosphohydroxythreonine aminotransferase (Lam and Winkler, J. Bacteriol. 172:6518-6528 (1990)), and activity on non-phosphorylated substrates could not be detected (Drewke et al., FEBS Lett. 390:179-182(1996)).

| Gene | Accession No. | GI No. | Organism |
|---|---|---|---|
| aspC | NP_415448.1 | 16128895 | Escherichia coli |
| AAT2 | P23542.3 | 1703040 | Saccharomyces cerevisiae |
| ASP5 | P46248.2 | 20532373 | Arabidopsis thaliana |
| Got2 | P00507 | 112987 | Rattus norvegicus |
| avtA | YP_026231.1 | 49176374 | Escherichia coli |
| serC | NP_415427.1 | 16128874 | Escherichia coli |

Another enzyme candidate is alpha-aminoadipate aminotransferase (EC 2.6.1.39), an enzyme that participates in lysine biosynthesis and degradation in some organisms. This enzyme interconverts 2-aminoadipate and 2-oxoadipate, using alpha-ketoglutarate as the amino acceptor. Gene candidates are found in Homo sapiens (Okuno et al., Enzyme Protein 47:136-148 (1993)) and Thermus thermophilus (Miyazaki et al., Microbiology 150:2327-2334 (2004)). The Thermus thermophilus enzyme, encoded by lysN, is active with several alternate substrates including oxaloacetate, 2-oxoisocaproate, 2-oxoisovalerate, and 2-oxo-3-methylvalerate.

| Gene | Accession No. | GI No. | Organism |
|---|---|---|---|
| lysN | BAC76939.1 | 31096548 | Thermus thermophilus |
| AadAT-II | Q8N5Z0.2 | 46395904 | Homo sapiens |

### 2.7.2.a Phosphotransferase (Carboxy Acceptor).

Phosphotransferase enzymes in the EC class 2.7.2 transform carboxylic acids to phosphonic acids with concurrent hydrolysis of one ATP. Step O of FIG. 62 involves the conversion of 4-hydroxybutyrate to 4-hydroxybutyryl-phosphate by such an enzyme. Butyrate kinase (EC 2.7.2.7) carries out the reversible conversion of butyryl-phosphate to butyrate during acidogenesis in C. acetobutylicum (Cary et al., Appl. Environ. Microbiol. 56:1576-1583 (1990)). This enzyme is encoded by either of the two buk gene products (Huang et al., J. Mol. Microbiol. Biotechnol. 2:33-38 (2000)). Other butyrate kinase enzymes are found in C. butyricum and C. tetanomorphum (Twarog and Wolfe, J. Bacteriol. 86:112-117 (1963)). Related enzyme isobutyrate kinase from Thermotoga maritima has also been expressed in E. coli and crystallized (Diao et al., Acta Crystallogr. D. Biol. Crystallogr. 59:1100-1102(2003); Diao and Hasson, J. Bacteriol. 191:2521-2529 (2009)). Aspartokinase catalyzes the ATP-dependent phosphorylation of aspartate and participates in the synthesis of several amino acids. The aspartokinase III enzyme in E. coli, encoded by lysC, has a broad substrate range, and the catalytic residues involved in substrate specificity have been elucidated (Keng and Viola, Arch. Biochem. Biophys. 335:73-81 (1996)). Two additional kinases in E. coli are also good candidates: acetate kinase and gamma-glutamyl kinase. The E. coli acetate kinase, encoded by ackA (Skarstedt and Silverstein, J. Biol. Chem. 251:6775-6783 (1976)), phosphorylates propionate in addition to acetate (Hesslinger et al., Mol. Microbiol. 27:477-492 (1998)). The E. coli gamma-glutamyl kinase, encoded by proB (Smith et al., J. Bacteriol. 157:545-551(1984)), phosphorylates the gamma carbonic acid group of glutamate.

| Gene | Accession No. | GI No. | Organism |
|---|---|---|---|
| buk1 | NP_349675 | 15896326 | Clostridium acetobutylicum |
| buk2 | Q97II1 | 20137415 | Clostridium acetobutylicum |
| buk2 | Q9X278.1 | 6685256 | Thermotoga maritima |
| lysC | NP_418448.1 | 16131850 | Escherichia coli |
| ackA | NP_416799.1 | 16130231 | Escherichia coli |
| proB | NP_414777.1 | 16128228 | Escherichia coli |

Acetylglutamate kinase phosphorylates acetylated glutamate during arginine biosynthesis. This enzyme is not known to accept alternate substrates; however, several residues of the E. coli enzyme involved in substrate binding and phosphorylation have been elucidated by site-directed mutagenesis (Marco-Marin et al., J. Mol. Biol. 334:459-476 (2003); Ramon-Maiques et al., Structure 10:329-342 (2002)). The enzyme is encoded by argB in Bacillus subtilis and E. coli (Parsot et al., Gene 68:275-283 (1988)), and ARG5,6 in S. cerevisiae (Pauwels et al., Eur. J. Biochem. 270:1014-1024 (2003)). The ARG5,6 gene of S. cerevisiae encodes a polyprotein precursor that is matured in the mitochondrial matrix to become acetylglutamate kinase and acetylglutamylphosphate reductase.

| Gene | Accession No. | GI No. | Organism |
|---|---|---|---|
| argB | NP_418394.3 | 145698337 | Escherichia coli |
| argB | NP_389003.1 | 16078186 | Bacillus subtilis |
| ARG5,6 | NP_010992.1 | 6320913 | Saccharomyces cerevisiae |

### 2.8.3.a Coa Transferase.

The gene products of cat1, cat2, and cat3 of Clostridium kluyveri have been shown to exhibit succinyl-CoA (Step G, FIGS. 62 and 63), 4-hydroxybutyryl-CoA (Step T, FIG. 62), and butyryl-CoA acetyltransferase activity, respectively (Seedorf et al., Proc. Natl. Acad. Sci. USA 105:2128-2133 (2008); Sohling and Gottschalk, J Bacteriol 178:871-880 (1996)). Similar CoA transferase activities are also present in Trichomonas vaginalis (van Grinsven et al., J. Biol. Chem. 283:1411-1418 (2008)) and Trypanosoma brucei (Riviere et al., J. Biol. Chem. 279:45337-45346 (2004)).

| Gene | Accession No. | GI No. | Organism |
|---|---|---|---|
| cat1 | P38946.1 | 729048 | Clostridium kluyveri |
| cat2 | P38942.2 | 1705614 | Clostridium kluyveri |
| cat3 | EDK35586.1 | 146349050 | Clostridium kluyveri |
| TVAG_395550 | XP_001330176 | 123975034 | Trichomonas |
| | | | vaginalis G3 |
| Tb11.02.0290 | XP_828352 | 71754875 | Trypanosoma brucei |

An additionally useful enzyme for this type of transformation is acyl-CoA:acetate-CoA transferase, also known as acetate-CoA transferase (EC 2.8.3.8), which has been shown to transfer the CoA moiety to acetate from a variety of branched and linear acyl-CoA substrates, including isobutyrate (Matthies and Schink, Appl. Environ. Microbiol. 58:1435-1439 (1992)), valerate (Vanderwinkel et al., Biochem. Biophys. Res. Commun. 33:902-908 (1968)) and butanoate (Vanderwinkel, supra (1968)). This enzyme is encoded by atoA (alpha subunit) and atoD (beta subunit) in E. coli sp. K12 (Korolev et al., Acta Crystallogr. D Biol. Crystallogr. 58:2116-2121 (2002); Vanderwinkel, supra (1968)). Similar enzymes exist in Corynebacterium glutamicum ATCC 13032 (Duncan et al., Appl. Environ. Microbiol. 68:5186-5190 (2002)), Clostridium acetobutylicum (Cary et al., Appl. Environ. Microbiol. 56:1576-1583 (1990); Wiesenborn et al., Appl. Environ. Microbiol. 55:323-329 (1989)), and Clostridium saccharoperbutylacetonicum (Kosaka et al., Biosci. Biotechnol. Biochem. 71:58-68 (2007)).

| Gene | Accession No. | GI No. | Organism |
|---|---|---|---|
| atoA | P76459.1 | 2492994 | Escherichia coli K12 |
| atoD | P76458.1 | 2492990 | Escherichia coli K12 |
| actA | YP_226809.1 | 62391407 | Corynebacterium glutamicum |
| cg0592 | YP_224801.1 | 62389399 | Corynebacterium glutamicum |
| ctfA | NP_149326.1 | 15004866 | Clostridium acetobutylicum |
| ctfB | NP_149327.1 | 15004867 | Clostridium acetobutylicum |
| ctfA | AAP42564.1 | 31075384 | Clostridium |
| | | | saccharoperbutylacetonicum |
| ctfB | AAP42565.1 | 31075385 | Clostridium |
| | | | saccharoperbutylacetonicum |

The glutaconate-CoA-transferase (EC 2.8.3.12) enzyme from anaerobic bacterium Acidaminococcus fermentans reacts with diacid glutaconyl-CoA and 3-butenoyl-CoA (Mack and Buckel, FEBS Lett. 405:209-212(1997)). The genes encoding this enzyme are gctA and gctB. This enzyme has reduced but detectable activity with other CoA derivatives including glutaryl-CoA, 2-hydroxyglutaryl-CoA, adipyl-CoA and acrylyl-CoA (Buckel et al., Eur. J. Biochem. 118:315-321(1981)). The enzyme has been cloned and expressed in E. coli(Mac et al., Eur. J. Biochem. 226:41-51 (1994)).

| Gene | Accession No. | GI No. | Organism |
|---|---|---|---|
| gctA | CAA57199.1 | 559392 | Acidaminococcus fermentans |
| gctB | CAA57200.1 | 559393 | Acidaminococcus fermentans |

### 3.1.2.a Coa Hydrolase.

Enzymes in the 3.1.2 family hydrolyze acyl-CoA molecules to their corresponding acids. However, such enzymes can be modified to empart CoA-ligase or synthetase functionality if coupled to an energy source such as a proton pump or direct ATP hydrolysis. Several eukaryotic acetyl-CoA hydrolases (EC 3.1.2.1) have broad substrate specificity. For example, the enzyme from Rattus norvegicus brain (Robinson et al., Biochem. Biophys. Res. Commun. 71:959-965 (1976)) can react with butyryl-CoA, hexanoyl-CoA and malonyl-CoA. Though its sequence has not been reported, the enzyme from the mitochondrion of the pea leaf also has a broad substrate specificity, with demonstrated activity on acetyl-CoA, propionyl-CoA, butyryl-CoA, palmitoyl-CoA, oleoyl-CoA, succinyl-CoA, and crotonyl-CoA (Zeiher and Randall, Plant. Physiol. 94:20-27 (1990)). The acetyl-CoA hydrolase, ACH1, from S. cerevisiae represents another candidate hydrolase (Buu et al., J. Biol. Chem. 278:17203-17209 (2003)).

| Gene | Accession No. | GI No. | Organism |
|---|---|---|---|
| acot12 | NP_570103.1 | 18543355 | Rattus norvegicus |
| ACH1 | NP_009538 | 6319456 | Saccharomyces cerevisiae |

Another candidate hydrolase is the human dicarboxylic acid thioesterase, acot8, which exhibits activity on glutaryl-CoA, adipyl-CoA, suberyl-CoA, sebacyl-CoA, and dodecanedioyl-CoA (Westin et al., J. Biol. Chem. 280:38125-38132 (2005)) and the closest E. coli homolog, tesB, which can also hydrolyze a broad range of CoA thioesters (Naggertet al., J. Biol. Chem. 266:11044-11050(1991)). A similar enzyme has also been characterized in the rat liver (Deana, Biochem. Int. 26:767-773 (1992)). Other potential E. coli thioester hydrolases include the gene products of tesA (Bonner and Bloch, J. Biol. Chem. 247:3123-3133 (1972)), ybgC (Kuznetsova et al., FEMS Microbiol. Rev. 29:263-279 (2005); Zhuang et al., FEBS Lett. 516:161-163 (2002)), paaI (Song et al., J. Biol. Chem. 281:11028-11038 (2006)), and ybdB (Leduc et al., J. Bacteriol. 189:7112-7126 (2007)).

| Gene | Accession No. | GI No. | Organism |
|---|---|---|---|
| acot8 | CAA15502 | 3191970 | Homo sapiens |
| tesB | NP_414986 | 16128437 | Escherichia coli |
| acot8 | NP_570112 | 51036669 | Rattus norvegicus |
| tesA | NP_415027 | 16128478 | Escherichia coli |
| ybgC | NP_415264 | 16128711 | Escherichia coli |
| paaI | NP_415914 | 16129357 | Escherichia coli |
| ybdB | NP_415129 | 16128580 | Escherichia coli |

Yet another candidate hydrolase is the glutaconate CoA-transferase from Acidaminococcus fermentans. This enzyme was transformed by site-directed mutagenesis into an acyl-CoA hydrolase with activity on glutaryl-CoA, acetyl-CoA and 3-butenoyl-CoA (Mack and Buckel, FEBS Lett. 405:209-212 (1997)). This indicates that the enzymes encoding succinyl-CoA:3-ketoacid-CoA transferases and acetoacetyl-CoA:acetyl-CoA transferases can also serve as candidates for this reaction step but would likely require certain mutations to change their function.

| Gene | Accession No. | GI No. | Organism |
|---|---|---|---|
| gctA | CAA57199.1 | 559392 | Acidaminococcus fermentans |
| gctB | CAA57200.1 | 559393 | Acidaminococcus fermentans |

Additional hydrolase enzymes include 3-hydroxyisobutyryl-CoA hydrolase which has been described to efficiently catalyze the conversion of 3-hydroxyisobutyryl-CoA to 3-hydroxyisobutyrate during valine degradation (Shimomura et al., J. Biol. Chem. 269:14248-14253 (1994)). Genes encoding this enzyme include hibch of Rattus norvegicus (Shimomura et al., supra (1994); Shimomura et al., Methods Enzymol. 324:229-240 (2000)) and Homo sapiens (Shimomura et al., supra (1994). Candidate genes by sequence homology include hibch of Saccharomyces cerevisiae and BC 2292 of Bacillus cereus.

| Gene | Accession No. | GI No. | Organism |
|---|---|---|---|
| hibch | Q5XIE6.2 | 146324906 | Rattus norvegicus |
| hibch | Q6NVY1.2 | 146324905 | Homo sapiens |
| hibch | P28817.2 | 2506374 | Saccharomyces cerevisiae |
| BC_2292 | AP09256 | 29895975 | Bacillus cereus |

### 4.1.1.a Carboxy-lyase.

Decarboxylation of Alpha-Keto Acids. Alpha-ketoglutarate decarboxylase (Step B, FIGS. 58, 62 and 63), 5-hydroxy-2-oxopentanoic acid decarboxylase (Step Z, FIG. 62), and 5-amino-2-oxopentanoate decarboxylase (Step R, FIG. 63) all involve the decarboxylation of an alpha-ketoacid. The decarboxylation of keto-acids is catalyzed by a variety of enzymes with varied substrate specificities, including pyruvate decarboxylase (EC 4.1.1.1), benzoylformate decarboxylase (EC 4.1.1.7), alpha-ketoglutarate decarboxylase and branched-chain alpha-ketoacid decarboxylase.

Pyruvate decarboxylase (PDC), also termed keto-acid decarboxylase, is a key enzyme in alcoholic fermentation, catalyzing the decarboxylation of pyruvate to acetaldehyde. The enzyme from Saccharomyces cerevisiae has a broad substrate range for aliphatic 2-keto acids including 2-ketobutyrate, 2-ketovalerate, 3-hydroxypyruvate and 2-phenylpyruvate (Davie et al., J. Biol. Chem. 267:16601-16606 (1992)). This enzyme has been extensively studied, engineered for altered activity, and functionally expressed in E. coli (Killenberg-Jabs et al., Eur. J. Biochem. 268:1698-1704 (2001); Li and Jordan, Biochemistry 38:10004-10012 (1999); ter Schure et al., Appl. Environ. Microbiol. 64:1303-1307 (1998)). The PDC from Zymomonas mobilus, encoded by pdc, also has abroad substrate range and has been a subject of directed engineering studies to alter the affinity for different substrates (Siegert et al., Protein Eng. Des. Sel. 18:345-357 (2005)). The crystal structure of this enzyme is available (Killenberg-Jabs et al., Eur. J. Biochem. 268:1698-1704 (2001)). Other well-characterized PDC candidates include the enzymes from Acetobacter pasteurians (Chandra et al., Arch. Microbiol. 176:443-451(2001)) and Khuyveromyces lactis (Krieger et al., Eur. J. Biochem. 269:3256-3263 (2002)).

| Gene | Accession No. | GI No. | Organism |
|---|---|---|---|
| pdc | P06672.1 | 118391 | Zymomonas mobilus |
| pdc1 | P06169 | 30923172 | Saccharomyces cerevisiae |
| pdc | AM21208 | 20385191 | Acetobacter pasteurians |
| pdc1 | Q12629 | 52788279 | Kluyveromyces lactis |

Like PDC, benzoylformate decarboxylase (EC 4.1.1.7) has a broad substrate range and has been the target of enzyme engineering studies. The enzyme from Pseudomonas putida has been extensively studied and crystal structures of this enzyme are available (Hasson et al., Biochemistry 37:9918-9930 (1998); Polovnikova et al., Biochemistry 42:1820-1830 (2003). Site-directed mutagenesis of two residues in the active site of the Pseudomonas putida enzyme altered the affinity (Km) of naturally and non-naturally occurring substrates (Siegert et al., Protein Eng. Des. Sel. 18:345-357 (2005)). The properties of this enzyme have been further modified by directed engineering (Lingen et al., Protein Eng. 15:585-593 (2002); Lingen et al., Chembiochem. 4:721-726 (2003)). The enzyme from Pseudomonas aeruginosa, encoded by mdlC, has also been characterized experimentally (Barrowman et al., FEMS Microbiol. Lett. 34:57-60 (1986)). Additional gene candidates from Pseudomonas stutzeri, Pseudomonas fluorescens and other organisms can be inferred by sequence homology or identified using a growth selection system developed in Pseudomonas putida (Henning et al., Appl. Environ. Microbiol. 72:7510-7517(2006)).

| Gene | Accession No. | GI No. | Organism |
|---|---|---|---|
| mdlC | P20906.2 | 3915757 | Pseudomonas putida |
| mdlC | Q9HUR2.1 | 81539678 | Pseudomonas aeruginosa |
| dpgB | ABN80423.1 | 126202187 | Pseudomonas stutzeri |
| ilvB-1 | YP_260581.1 | 70730840 | Pseudomonas fluorescens |

A third enzyme capable of decarboxylating 2-oxoacids is alpha-ketoglutarate decarboxylase (KGD). The substrate range of this class of enzymes has not been studied to date. The KDC from Mycobacterium tuberculosis (Tian et al., Proc. Natl. Acad. Sci. USA 102:10670-10675 (2005)) has been cloned and functionally expressed. However, it is not an ideal candidate for strain engineering because it is large (˜130 kD) and GC-rich. KDC enzyme activity has been detected in several species of rhizobia including Bradyrhizobium japonicum and Mesorhizobium loti (Green et al., J. Bacteriol. 182:2838-2844 (2000). Although the KDC-encoding gene(s) have not been isolated in these organisms, the genome sequences are available, and several genes in each genome are annotated as putative KDCs. A KDC from Euglena gracilis has also been characterized, but the gene associated with this activity has not been identified to date (Shigeoka and Nakano, Arch. Biochem. Biophys. 288:22-28 (1991)). The first twenty amino acids starting from the N-terminus were sequenced (MTYKAPVKDVKFLLDKVFKV; SEQ ID NO:45)(Shigeoka and Nakano, Arch. Biochem. Biophys. 288:22-28 (1991)). The gene can be identified by testing candidate genes containing this N-terminal sequence for KDC activity.

| Gene | Accession No. | GI No. | Organism |
|---|---|---|---|
| kgd | O50463.4 | 160395583 | Mycobacterium tuberculosis |
| kgd | NP_767092.1 | 27375563 | Bradyrhizobium japonicum |
| kgd | NP_105204.1 | 13473636 | Mesorhizobium loti |

A fourth candidate enzyme for catalyzing this reaction is branched chain alpha-ketoacid decarboxylase (BCKA). This class of enzyme has been shown to act on a variety of compounds varying in chain length from 3 to 6 carbons (Oku and Kaneda, J. Biol. Chem. 263:18386-18396 (1988); Smit et al., B. A., J. E. Hylckama Vlieg, W. J. Engels, L. Meijer, J. T. Wouters, and G. Smit. Identification, cloning, and characterization of a Lactococcus lactis branched-chain alpha-keto acid decarboxylase involved in flavor formation. Appl. Environ. Microbiol. 71:303-311(2005)). The enzyme in Lactococcus lactis has been characterized on a variety of branched and linear substrates including 2-oxobutanoate, 2-oxohexanoate, 2-oxopentanoate, 3-methyl-2-oxobutanoate, 4-methyl-2-oxobutanoate and isocaproate (Smit et al., Appl. Environ. Microbiol. 71:303-311(2005)). The enzyme has been structurally characterized (Berg et al., Science 318:1782-1786(2007)). Sequence alignments between the Lactococcus lactis enzyme and the pyruvate decarboxylase of Zymomonas mobilus indicate that the catalytic and substrate recognition residues are nearly identical (Siegert et al., Protein Eng. Des. Sel. 18:345-357 (2005)), so this enzyme would be a promising candidate for directed engineering. Decarboxylation of alpha-ketoglutarate by a BCKA was detected in Bacillus subtilis; however, this activity was low (5%) relative to activity on other branched-chain substrates (Oku and Kaneda. Biosynthesis of branched-chain fatty acids in Bacillus subtilis. A decarboxylase is essential for branched-chain fatty acid synthetase. J. Biol. Chem. 263:18386-18396 (1988)), and the gene encoding this enzyme has not been identified to date. Additional BCKA gene candidates can be identified by homology to the Lactococcus lactis protein sequence. Many of the high-scoring BLASTp hits to this enzyme are annotated as indolepyruvate decarboxylases (EC 4.1.1.74). Indolepyruvate decarboxylase (IPDA) is an enzyme that catalyzes the decarboxylation of indolepyruvate to indoleacetaldehyde in plants and plant bacteria.

| Gene | Accession No. | GI No. | Organism |
|---|---|---|---|
| kdcA | AAS49166.1 | 44921617 | Lactococcus lactis |

Recombinant branched chain alpha-keto acid decarboxylase enzymes derived from the E1 subunits of the mitochondrial branched-chain keto acid dehydrogenase complex from Homo sapiens and Bos taurus have been cloned and functionally expressed in E. coli (Davie et al., J. Biol. Chem. 267:16601-16606 1992); Wynn et al., J. Biol. Chem. 267:1881-1887 (1992); Wynn et al., J. Biol. Chem. 267:12400-12403 (1992)). In these studies, the authors found that co-expression of chaperonins GroEL and GroES enhanced the specific activity of the decarboxylase by 500-fold (Wynn et al., J. Biol. Chem. 267:12400-12403 (1992)). These enzymes are composed of two alpha and two beta subunits.

| Gene | Accession No. | GI No. | Organism |
|---|---|---|---|
| BCKDHB | NP_898871.1 | 34101272 | Homo sapiens |
| BCKDHA | NP_000700.1 | 11386135 | Homo sapiens |
| BCKDHB | P21839 | 115502434 | Bos taurus |
| BCKDHA | P11178 | 129030 | Bos taurus |

Decarboxylation of Alpha-Keto Acids. Several omithine decarboxylase (Step U, FIG. 63) enzymes also exhibit activity on lysine and other similar compounds. Such enzymes are found in Nicotiana glutinosa (Lee and Cho, Biochem. J. 360:657-665 (2001)), Lactobacillus sp. 30a (Guirard and Snell, J. Biol. Chem. 255:5960-5964 (1980)) and Vibrio vulnificus (Lee et al., J. Biol. Chem. 282:27115-27125 (2007)). The enzymes from Lactobacillus sp. 30a (Momany et al., J. Mol. Biol. 252:643-655 (1995)) and vulnificus have been crystallized. The V. vulnificus enzyme efficiently catalyzes lysine decarboxylation, and the residues involved in substrate specificity have been elucidated (Lee et al., J. Biol. Chem. 282:27115-27125 (2007)). A similar enzyme has been characterized in Trichomonas vaginalis, but the gene encoding this enzyme is not known (Yarlett et al., Biochem. J. 293 (Pt 2):487-493 (1993)).

| Gene | Accession No. | GI No. | Organism |
|---|---|---|---|
| AF323910.1:1 . . . 1299 | AAG45222.1 | 12007488 | Nicotiana |
| | | | glutinosa |
| odc1 | P43099.2 | 1169251 | Lactobacillus |
| | | | sp. 30a |
| VV2_1235 | NP_763142.1 | 27367615 | Vibrio vulnificus |

Glutamate decarboxylase enzymes (Step L, FIGS. 62 and 63) are also well-characterized. Exemplary glutamate decarboxylases can be found in E. coli (De Biase et al., Protein Expr. Purif. 8:430438 (1996)), S. cerevisiae (Coleman et al., J. Biol. Chem. 276:244-250 (2001)), and Homo sapiens (Bu et al., Proc. Natl. Acad. Sci. USA 89:2115-2119 (1992); Bu and Tobin, Genomics 21:222-228 (1994)).

| Gene | Accession No. | GI No. | Organism |
|---|---|---|---|
| GAD1 | NP_000808 | 58331246 | Homo sapiens |
| GAD2 | NP_001127838 | 197276620 | Homo sapiens |
| gadA | NP_417974 | 16131389 | Escherichia coli |
| gadB | NP_416010 | 16129452 | Escherichia coli |
| GAD1 | NP_013976 | 6323905 | Saccharomyces cerevisiae |

Lysine decarboxylase (EC 4.1.1.18) catalyzes the decarboxylation of lysine to cadaverine. Two isozymes of this enzyme are encoded in the E. coli genome by genes cadA and ldcC. CadA is involved in acid resistance and is subject to positive regulation by the cadC gene product (Lemonnier and Lane, Microbiology 144 (Pt 3):751-760 (1998)). CadC accepts hydroxylysine and S-aminoethylcysteine as alternate substrates, and 2-Aminopimelate and 6-ACA act as competitive inhibitors to this enzyme (Sabo et al., Biochemistry 13:662-670 (1974)). Directed evolution or other enzyme engineering methods can be utilized to increase the activity for this enzyme to decarboxylate 2-aminopimelate. The constitutively expressed ldc gene product is less active than CadA (Lemonnier and Lane, Microbiology 144 (Pt 3):751-760 (1998)). A lysine decarboxylase analogous to CadA was recently identified in Vibrio parahaemolyticus (Tanaka et al., J. Appl. Microbiol. 104:1283-1293 (2008)). The lysine decarboxylase from Selenomonas ruminantium, encoded by ldc, bears sequence similarity to eukaryotic ornithine decarboxylases, and accepts both L-lysine and L-omithine as substrates (Takatsuka et al., Biosci. Biotechnol. Biochem. 63:1843-1846 (1999)). Active site residues were identified and engineered to alter the substrate specificity of the enzyme (Takatsuka et al., J. Bacteriol. 182:6732-6741(2000)).

| Gene | Accession No. | GI No. | Organism |
|---|---|---|---|
| cadA | AAA23536.1 | 145458 | Escherichia coli |
| ldcC | AAC73297.1 | 1786384 | Escherichia coli |
| ldc | O50657.1 | 13124043 | Selenomonas ruminantium |
| cadA | AB124819.1 | 44886078 | Vibrio parahaemolyticus |

### 6.2.1.a Coa Synthetase.

CoA synthetase or ligase reactions are required by Step I of FIGS. 62 and 63, and Step V of FIG. 62. Succinate or 4-hydroxybutyrate are the required substrates. Exemplary genes encoding enzymes likely to cany out these transformations include the sucCD genes of E. coli, which naturally form a succinyl-CoA synthetase complex. This enzyme complex naturally catalyzes the formation of succinyl-CoA from succinate with the concomitant consumption of one ATP, a reaction which is reversible in vivo (Buck et al., Biochem. 24:6245-6252(1985)).

| Gene | Accession No. | GI No. | Organism |
|---|---|---|---|
| sucC | NP_415256.1 | 16128703 | Escherichia coli |
| sucD | AAC73823.1 | 1786949 | Escherichia coli |

Additional exemplary CoA-ligases include the rat dicarboxylate-CoA ligase for which the sequence is yet uncharacterized (Vamecq et al., Biochemical J. 230:683-693 (1985)), either of the two characterized phenylacetate-CoA ligases from P. chrysogenum (Lamas-Maceiras et al., Biochem. J. 395:147-155 (2005); Wang et al., Biochem Biophy Res Commun 360(2):453-458 (2007)), the phenylacetate-CoA ligase from Pseudomonas putida (Martinez-Blanco et al., J. Biol. Chem. 265:7084-7090 (1990)), and the 6-carboxyhexanoate-CoA ligase from Bacillus subtilis (Boweret al., J. Bacteriol. 178(14):4122-4130 (1996)). Additional candidate enzymes are acetoacetyl-CoA synthetases from Mus musculus (Hasegawa et al., Biochim. Biophys. Acta 1779:414-419 (2008)) and Homo sapiens (Ohgami et al., Biochem. Pharmacol. 65:989-994 (2003)), which naturally catalyze the ATP-dependant conversion of acetoacetate into acetoacetyl-CoA. 4-Hydroxybutyryl-CoA synthetase activity has been demonstrated in Metallosphaera sedula (Berg et al., Science 318:1782-1786 (2007)). This function has been tentatively assigned to the Msed_1422 gene.

| Gene | Accession No. | GI No. | Organism |
|---|---|---|---|
| phl | CAJ15517.1 | 77019264 | Penicillium chrysogenum |
| phlB | ABS19624.1 | 152002983 | Penicillium chrysogenum |
| paaF | AAC24333.2 | 22711873 | Pseudomonas putida |
| bioW | NP_390902.2 | 50812281 | Bacillus subtilis |
| AACS | NP_084486.1 | 21313520 | Mus musculus |
| AACS | NP_076417.2 | 31982927 | Homo sapiens |
| Msed_1422 | YP_001191504 | 146304188 | Metallosphaera sedula |

ADP-forming acetyl-CoA synthetase (ACD, EC 6.2.1.13) is another candidate enzyme that couples the conversion of acyl-CoA esters to their corresponding acids with the concurrent synthesis of ATP. Several enzymes with broad substrate specificities have been described in the literature. ACD I from Archaeoglobus fulgidus, encoded by AF1211, was shown to operate on a variety of linear and branched-chain substrates including acetyl-CoA, propionyl-CoA, butyryl-CoA, acetate, propionate, butyrate, isobutyrate, isovalerate, succinate, fumarate, phenylacetate, indoleacetate (Musfeldt et al., J. Bacteriol. 184:636-644 (2002)). The enzyme from Haloarcula marismortui (annotated as a succinyl-CoA synthetase) accepts propionate, butyrate, and branched-chain acids (isovalerate and isobutyrate) as substrates, and was shown to operate in the forward and reverse directions (Brasen et al., Arch. Microbiol. 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen et al., supra (2004)). The enzymes from A. fulgidus, H. marismortui and P. aerophilum have all been cloned, functionally expressed, and characterized in E. coli (Musfeldt et al., supra; Brasen et al., supra (2004)).

| Gene | Accession No. | GI No. | Organism |
|---|---|---|---|
| AF1211 | NP_070039.1 | 11498810 | Archaeoglobus fulgidus |
| | | | DSM 4304 |
| scs | YP_135572.1 | 55377722 | Haloarcula marismortui |
| | | | ATCC 43049 |
| PAE3250 | NP_560604.1 | 18313937 | Pyrobaculum aerophilum |
| | | | str. IM2 |

Production of BDO Utilizing Carboxylic Acid Reductase. Described below is the generation of a microbial organism that produces 1,4-butanediol using carboxylic acid reductase enzymes. See corresponding example in WO2013/186402.

These results show that various carboxylic acid reductases can function in a BDO pathway to produce BDO.

### Example XXIV

### Metabolic Modifications to Improve Engineered Microorganism Characteristics

This example describes additional metabolic modifications to improve characteristics of an engineered microorganism. Such improved characteristics include, but are not limited to, increased product yield, decreased production of by-products, improved growth characteristics of the engineered microorganisms, including improved characteristics for scaleup production, and the like. See corresponding example in WO2013/186402.

Cultivation conditions for bottles. Twenty-milliliter bottle cultivations for metabolite production or bioconversion were performed in M9 minimal salts medium (6.78 g/L Na₂HPO₄, 3.0 g/L KH₂PO₄, 0.5 g/L NaCl, 1.0 g/L NH₄Cl, 1 mM MgSO₄, 0.1 mM CaCl₂) supplemented with 10 mM NaHCO₃, 20 g/L D-glucose and 100 mM MOPS to improve the buffering capacity, 10 μg/ml thiamine and the appropriate antibiotics for plasmid maintenance. E. coli strain MG1655 lacIQ+ was grown anaerobically, and anaerobic conditions were obtained by flushing capped anaerobic bottles with nitrogen for at least 5 min. Microaerobic conditions were used for all other strains, which were established by initially flushing capped anaerobic bottles with nitrogen for 5 min, then piercing the septum with a 23G needle (Becton-Dickenson) after inoculation. The needle was kept in the bottle during growth to allow a small amount of air to enter. Protein expression was induced with 0.2 mM IPTG when the culture reached mid-log growth phase, unless otherwise indicated in the text.

Cultivation conditions for 96 well-plates. All the cultures in 96 well-plates were grown in 1.2 ml of M9 medium (composition provided above) with MOPS and appropriate antibiotics. Carbon source in the form of 5% glucose was also added. Microaerobic conditions were obtained by covering the plates with two gas-permeable adhesive seals. The edges of the seal were taped to minimize evaporation. All the cultures were grown at 37° C.

Bacterial strains and plasmids. Genomic DNA from bacterial strains was isolated with the PureLink Genomic DNA Mini Kit (Invitrogen) according to the manufacturer's instructions. Recombinant DNA manipulations were conducted as described (Sambrook et al., T. Molecular Cloning: A Laboratory Manual, Cold Spring Harbor Laboratory, Cold Spring Harbor, New York, (1989). Genes and open reading frames in the study were amplified from appropriate genomic DNA templates using the high-fidelity KOD DNA polymerase enzyme (EMD Chemicals; Billerica MA). Analytical PCR experiments for genotyping and sequencing were conducted according to standard molecular biology protocols with Taq polymerase (Promega). DNA sequencing was provided by Genewiz (South Plainfield NJ).

Procedure for genetic manipulations. Development of expression vectors for BDO pathway. Vector backbones were obtained from Dr. Rolf Lutz of Expressys (expressys.de). The vectors and strains are based on the pZ Expression System described previously (Lutz and Bujard, Nucleic Acids Res. 25:1203-1210 (1997).). Vectors obtained were pZE13luc, pZA33luc, pZS*13luc and pZE22luc and contained the luciferase gene as a stuffer fragment. To replace the luciferase stuffer fragment with a lacZ-alpha fragment flanked by appropriate restriction enzyme sites, the luciferase stuffer fragment was first removed from each vector by digestion with EcoRI and XbaI. The lacZ-alpha fragment was PCR amplified from pUC19 with the following primers:

lacZalpha-RI
(SEQ ID NO: 1)
5′GACGAATTCGCTAGCAAGAGGAGAAGTCGACATGTCCAATTCACTGG
CCGTCGTTTTAC3′;
lacZalpha 3′BB
(SEQ ID NO: 2)
5′-GACCCTAGGAAGCTTTCTAGAGTCGACCTATGCGGCATCAGAGCA
GA-3′.

This generated a fragment with a 5′ end of EcoRI site, NheI site, a Ribosomal Binding Site, a SalI site and the start codon. The 3′ end of the fragment contained the stop codon, XbaI, HindIII, and AvrII sites. The PCR product was digested with EcoRI and AvrII and ligated into the base vectors digested with EcoRI and XbaI (XbaI and AvrII have compatible ends and generate a non-site). Because NheI and XbaI restriction enzyme sites generate compatible ends that can be ligated together (but generate a site after ligation that is not digested by either enzyme), the genes cloned into the vectors could be “Biobricked” together (openwetware.org/wiki/Synthetic_Biology:BioBricks). Briefly, this method allows joining an unlimited number of genes into the vector using the same 2 restriction sites, as long as the sites do not appear internal to the genes, because the sites between the genes are destroyed after each addition. Initially, expression was low from these vectors, and they were subsequently modified using the Phusion® Site-Directed Mutagenesis Kit (NEB, Ipswich, MA) to insert the spacer sequence AATTAA between the EcoRI and NheI sites. This eliminated a putative stem loop structure in the RNA that bound the RBS and start codon.

All vectors have the pZ designation followed by letters and numbers indicating the origin of replication, antibiotic resistance marker and promoter/regulatory unit. The origin of replication is the second letter and is denoted by E for ColE1, A for p15A and S for pSC101 (as well as a lower copy number version of pSC101 designated S*)—based origins. The first number represents the antibiotic resistance marker (1 for Ampicillin, 2 for Kanamycin, 3 for Chloramphenicol). The final number defines the promoter that regulated the gene of interest (1 for PLtetO-1, 2 for PLlacO-1 and 3 for PAllacO-1) and each of these promoters became activated by its corresponding inducer molecule (pLtetO can be induced by tetracycline; pLlacO-1 and pAllacO-1 can be induced by IPTG). Three base vectors, pZS*13S, pZA33S and pZE13S, were then designed and constructed to serve as “inducible” plasmid vectors.

In addition to the “inducible” promoters mentioned above, a set of“constitutive” promoters were sampled from the Registry (partsregistry.org). Each of these “constitutive” promoters was then introduced into the pZS*13S vector backbone to replace the pAllacO-1 inducible promoter via Sequence and Ligation Independent Cloning (SLIC) method described by Li & Eledge (Nature Methods 2007, 4:251-256). Of these sampled “constitutive” promoters (p100, p104, p105, p107, p108, p111, p115 & p119), experiments were carried out to establish an order of promoter strength that was verified by protein expression levels. For these experiments, both “inducible” and “constitutive” plasmid vectors were employed, modified for the biobricks and SLIC insertions as discussed above. To further fine-tune protein expression levels of some overly expressed proteins, ribosomal binding site (RBS) in between promoter and gene coding sequence was modified accordingly using the RBS calculator (salis.psu.edu/software/).

The SLIC primers used for inserting a list of genes into the pZS*13S vector backbone are listed below. The lower case marks the sequences annealing to a vector backbone while the upper case marks the sequences annealing to the coding region of a gene.

1. ppc 525
Forward SLIC primer:
(SEQ ID NO: 100)
gaggagaagtcgacATGAACGAACAATATTCCGCATTGCG
Reverse SLIC primer:
(SEQ ID NO: 101)
ggaagctttctagaTTAGCCGGTATTACGCATACCTGCC
2. sucAB-lpdA
Forward SLIC primer:
(SEQ ID NO: 102)
taagctagcaagaggagaagtcgacATGCAGAACAGCGCTTTGAAAG
Reverse SLIC primer:
(SEQ ID NO: 103)
gcctctaggaagctttctagaTTACTTTTTCTTCGCTTTGGCG
3. galP 1120
Forward SLIC primer:
(SEQ ID NO: 104)
ctagcaagaggagaagtcgacATGCCTGACGCTAAAAAACAGGGGCGGT
Reverse SLIC primer:
(SEQ ID NO: 105)
ctaggaagctttctagagtcgTTAATCGTGAGCGCCTATTTCGCGCAG
4. glk 1123
Forward SLIC primer:
(SEQ ID NO: 106)
ctagcaagaggagaagtcgacATGACAAAGTATGCATTAGTCGGTGATG
TG
Reverse SLIC primer:
(SEQ ID NO: 107)
ctaggaagctttctagagtcgTTACAGAATGTGACCTAAGGTCTGGCGT
AAATG
5. glf 1786
Forward SLIC primer:
(SEQ ID NO: 108)
ctagcaagaggagaagtcgacATGAGTTCTGAAAGTAGTCAGGGTCTAG
TC
Reverse SLIC primers:
(SEQ ID NO: 109)
ctaggaagctttctagagtcgTTACTTCTGGGAGCGCCACATCTC

For the work discussed below, three base vectors, pZS*13S, pZA33S and pZE13S, modified for the biobricks insertions as discussed above, were employed.

The following technique describes the construction of the incW plasmids, pPSX13 and pPSX23R. The original incW plasmid backbone (pPSX) was obtained from Dr. John M. Pemberton (Sarovich and Pemberton, Plasmid 57:306-313 (2007)). The pPSX plasmid was modified to insert either an ampicillin marker (bla gene), designated as pPSX13, into a BamHI restriction enzyme site or an kanamycin marker and a R6k replication origin, designated as pPSX23R, in between BamHI and SacI restriction enzyme sites. Two similar incW plasmids were also constructed to include the ald-adh gene cassette amplified from pZS*13S-ald-adh. For pPSX13-ald-adh ampicillin-resistant plasmid, the bla-ald-adh gene cassette was inserted into a BamHI restriction enzyme site of pPSX13. To construct pPSX23R-ald-adh kanamycin resistant plasmid, the ampicillin marker (bla gene) was replaced with the “kanamycine marker-R6k replication origine” cassette in between XhoI and SacI restriction enzyme sites of pPSX23R.

The genes 033B, 1210, and 956 were inserted in the mini-F-plasmid backbone pKLJ33S as follows: PCR primers were designed to amplify the genes 033B (033BFOR and 033BREV), 1210C (1210CFOR and 1210CREV), and 956 (956FOR and 956REV) from appropriate templates. These primers included the immediate 5′ (FOR) and 3′(REV) ends of each gene as denoted in upper case. Each primer also included a tail that was directly homologous to the promoter region (FOR) and terminator region (REV) of plasmid pZA33S. A linear fragment of pZA33S with the promoter region and terminator regions at either extreme end was amplified using Primers pZ-5′ and pZ-3′ and circular pZA33S as a template. The pZA33S linear fragment was combined with each of the 033B, 1210C, and 956 gene fragments and joined using sequence- and ligation-independent cloning to form the corresponding plasmids pZA33S-033B, pZA33S-1210C, and pZA33S-956.

| Primer | | SEQ ID |
|---|---|---|
| Designation | Primer Sequence | NO |
| 033BFOR | tagcaagaggagaagtcgacATGGACTGGAAGAAGATCTATGAAG | 110 |
| 033BREV | cctctaggaagatttctagaTTAGAATGCCGCGTTGAAG | 111 |
| 1210CFOR | tagcaagaggagaagtcgacATGAGCTGGCAAGAACTGTATC | 112 |
| 1210CREV | cctctaggaagctttctagaTTAATATTTCTCTTTAAAGCGCTTTTC | 113 |
| 956FOR | tagcaagaggagaagtcgacATGGCACGTTTTACTTTACCAAG | 114 |
| 956REV | cctctaggaagctttctagaTTACAAATTAACTTTAGTTCCATAGTATGTGC | 115 |
| 034Cmfor | accggtaaacgactcagcagcctgacgcactggcgatattattttgccttct | 116 |
| | cctcaccacagaatgttctgccacctgaCGATATCAAATTACGCCCCG | |
| 034Cmrev | tgcttatccacaacattttgcgcacggttatgtggacaaaatacctggttac | 117 |
| | ccaggccgtgccggcacgttaaccgggcCCTAGGTCTAGGGCGGCGGATTTG | |

Primers 034Cm for and 034Cmrev were used to amplify the 033B, 1210C, and 956 genes from the appropriate pZA33S plasmid background. These primers were designed to amplify the chloramphenicol resistance gene, pA1 promoter, particular gene of interest, and terminator elements from plasmid pZA33S (with the sequences denoted in upper case) while tail sequences (denoted in lower case) corresponded to regions flanking but not including the ampicillin resistance gene, arabinose responsive promoter and terminator sequences of the mini-F-plasmid pKLJ12 (Jones and Keasling, Biotechnol. Bioengineer 59:659-665 (1998)).

An E. coli DP10B strain containing plasmid pKLJ12 and temperature-sensitive Red/ET helper plasmid pREDET (Tet)(GeneBridges GmbH, Heidelberg Germany) was inoculated into LB medium containing tetracycline at 10 μg/ml and ampicillin at 100 μg/ml final concentrations and incubated for 3 hours at 30° C. with shaking until cells were turbid. Arabinose to induce expression of the Red/ET recombination functions was added to a final concentration of 0.3%, the temperature was shifted to 37° C. and the cells were allowed to incubate for an additional hour with shaking. After being made electrocompetent by repeated centrifugation and resuspension in ice-cold sterile double-distilled water, these cells were mixed with 034Cmfor/034Cmrev PCR amplifications from the pZA33S-033B, pZA33S-1210C, or pZA33S-956 plasmids in a 1 mm cuvette and then subjected to electroporation at 1800 kV, 25 μF, 200Ω. Electroporated cells were then added to SOC growth medium, incubated at 37° C. without shaking, and then spread on LB solid medium containing chloramphenicol at 10 μg/ml final concentration and incubated at 37° C. for 18 to 24 hours. Resulting chloramphenicol-resistant single colonies were then scored for growth on solid LB media containing either tetracycline at 10 μg/ml or the ampicillin at 100 μg/ml. Colonies that were shown to be chloramphenicol resistant, tetracycline sensitive, and ampicillin sensitive were verified by PCR and DNA sequencing to contain new mini-F-plasmids that had completely exchanged the ampicillin resistance gene/arabinose responsive promoter/terminator of pKLJ12 with the chloramphenicol resistance gene/PA1 promoter/gene-of-interest/terminator of pZA33S by RED/ET-mediated homologous recombination, resulting in the new mini-F-plasmids pKLJ33S-033B, pKLJ335-1210C, pKLJ33S-956.

Chromosomal replacement of genes using the sacB gene. The primary method used for the insertion or deletion of genes in the chromosome of E. coli was based on the utilization of the sacB gene from Bacillus subtilis (Gay et al., J. Bacteriol. 153:1424-1431(1983)). The vector used is pRE118 (ATCC87693) deleted of the oriT and IS sequences. The resulting vector (3.6 kb in size and carrying the kanamycin resistance gene) was sequenced and called pRE118-V2. All cloning of fragments was done into the restriction sites KpnI and PstI of pRE118-V2, unless notified. All PCR amplification used genomic DNA from E. coli MG1655 (ATCC47076) as DNA template. The first integration event in the chromosome was selected on LB agar plates containing Kanamycin (25 or 50 mg/L). Correct insertions were verified by PCR using 2 primers: one located outside the region of insertion and one in the kanamycin gene (5′-aggcagttccataggatggc-3′)(SEQ ID NO:118). Clones with the correct insertion were selected for resolution. They were sub-cultured twice in plain liquid LB at the desired temperature and serial dilutions were plated on LB-no salt-sucrose 10% plates. Clones that grew on sucrose containing plates were screened for the loss of the kanamycin resistance gene on LB-low salt agar medium and the deletion/insertion of the fragment of interest was verified by PCR and sequencing of the encompassing region.

This method was used for deleting appCB using the following primers:

| | | SEQ |
|---|---|---|
| Primer | | ID |
| Designation | Primer Sequence | NO |
| appCB Kpn fw | Ataataataggtaccggcggcgctggcgcagc | 119 |
| | ttgctgcg | |
| appCB BamHI rv | TATTATTATGGATCCAACCCGATAATGGTAGA | 120 |
| | TCTCCCTCT | |
| appCB BamHI fw | Ataataataggatccggagcagaaacaatgtg | 121 |
| | gtatttact | |
| appCB Pst rv | TATTATTATCTGCAGATGCTCTTTTTTATGCA | 122 |
| | TTACAAACTGC | |

As a result, the following nucleotides were deleted: 1,036,963->1,039,655

tesB and ybgC deletions were also made with aforementioned pRE-sacB-Kan vector. The primers used for making the deletion were: (5′-3′)

| Primer | | SEQ |
|---|---|---|
| Designation | Primer Sequence | ID NO |
| tesB-kpn-frw | taataataaggtaccaactgggcttgcttcactgg | 123 |
| tesB-bgl-rev | taataataaAGATCTGCCTCGACCGTTCAGGAAGG | 124 |
| tesB-bgl-frw | taataataaagatcttaactctccagtaacaaagctgc | 125 |
| tesB-Pst-rev | taataataaCTGCAGGCTATGTCACCACTTACGG | 126 |
| ybgC-Pst-frw | Tattattattcctgcaggcgtctttgttcttccgtcc | 127 |
| ybgC-Kpn-rev | tattattatCTTCGGTACCTTTAGCATCTGCTTCGGCC | 128 |
| ybgC-Bam-rev | tattattatGGATCCCGGTAATGCAACAAAAGTTAGAGC | 129 |
| ybgC-Bam-frw | tattattatggatccaaagtgactgacatgaatatccttgatttgttc | 130 |

Nucleotides deleted as a consequence were: tesB: 473,525<-474,385; ybgC: 773,975->774,379

An ndh deletion was also made with the standard pRE-sacB-Kan vector. The primers used for making the deletion were: (5′-3′)

| | | SEQ |
|---|---|---|
| Primer | | ID |
| Designation | Primer Sequence | NO |
| ndh-Bam-frw | taataataaggatcctacactggcggatgtggcat | 131 |
| | aaac | |
| ndh-Kpn-rev | taataatttggtacccATTCACAGTCACCAGGTAC | 132 |
| | AACG | |
| ndh-Bam-rev | taataataaGGATCCGAGAATAACATGAATGGTGC | 133 |
| | ATTG | |
| ndh-Pst-frw | taataatatctgcagatccacaaaaagccctggca | 134 |
| | attg | |

Nucleotides deleted as a result of the deletion were: 1,165,308->1,166,612

Deletions of yjgB, yqhD, yahK and adhP were introduced with the standard pRE-sacB-Kan vector. The primers used for making the deletion were: (5′-3′)

| Primer | | |
|---|---|---|
| Designation | Primer Sequence | SEQ ID NO |
| ygjB-Pst-frw | gataatcccccctgcagggagcggtaaatag | 135 |
| ygjB-Bam-rev | tattatTTTGGATCCTTCTCTGGTGTTGTTTGGG | 136 |
| yjgB-Bam-frw | tattattatggatccgcgtggtgttgaaagccg | 137 |
| yjgBKpn-rev | tattatAAAGGTACCGCCTCTTCCAGGTCAGTGAAGGG | 138 |
| yqhD-Kpn-frw | taataataaggtacccagttttggctatgccttaag | 139 |
| yqhD-Hind-rev | taataaTCATTAAGCTTGCTCCCTTTGCTGGGCC | 140 |
| yqhD-Hind-frw | taataataataagatttttacgcctcaaactttcg | 141 |
| yqhD-Pst-rev | taataataaATTGCCCTGCAGCGTAAGATTGTCGTTCAGGG | 142 |
| yahK-Kpn-frw | tattattattgtttggtacctctgtgccgct | 143 |
| yahK-Pst-rev | AATGTTCCACTCTGCAGGGATGATAATAAGGGG | 144 |
| yahK-Bam-frw | atcggatccaatcgcacactaacagactg | 145 |
| yahK-Bam-rev | ACAGCTTTGGATCCTCATTGTGTTTACTCCTGATTAGC | 146 |

| | | SEQ |
|---|---|---|
| Primer | | ID |
| Designation | Primer Sequence | NO |
| adhP XbaI fw | ataataatatctagagcagcaagccgcgcgg | 147 |
| | caggtggtcag | |
| adhP PstI rv | TATTATTATCTGCAGACGCCATCCTGATCCA | 148 |
| | TATGTATATGG | |
| adhP Bam up | TATTATTATGGATCCAGTTCCTCCTTTTCGG | 149 |
| rv | ATGATGTTCTG | |
| adhP Bam | ataataataggatccgaggcctttgctgcga | 150 |
| down fw | ctgccatgttc | |

Nucleotides deleted as a result of each of these deletions were: ygjB: 4,493,213<-4,494,232, yqhD: 3,153,377->3,154,540, yahK: 342,108->343,157, adhP: 1,550,852<-1,551,862.

Gene deletions using lambda-red mediated combination: Native genes cyoABCD were deleted from the chromosome by Red-mediated recombination (ncbi.nlm.nih.gov/pubmed/10829079).

For cyoABCD deletion, the following primers were used to amplify a chloramphenicol or kanamycin resistance gene from pKD3 or pKD4 (ncbi.nlm.nih.gov/pubmed/10829079), respectively, and were flanked by FRT sites and homologies to cyoABCD:

LK-cyo-F1
(SEQ ID NO: 151)
CGCCACAACCAGTGACACCC
LK-cyo-R1
(SEQ ID NO: 152)
GGGGTTTTAGTCGCCCTTTCTGGC
LK-cyo-IO-down
(SEQ ID NO: 153)
CGGGATCTGGTGGCGTTTAAAGTG
LK-cyo-IO-up
(SEQ ID NO: 154)
TGACGGCGTTTGTCTTCACCG

E. coli K-12MG1655 carrying the Red helper plasmid pKD46 was grown in 100 ml LB medium with ampicillin and 1 mM L-arabinose at 30° C. to an OD600 of 0.3, and electroporation-competent cells were prepared as described elsewhere (Sambrook et al., Molecular Cloning: A Laboratory Manual, Cold Spring Harbor Laboratory, Cold Spring Harbor, N, (1989)). A measure of 50 μl of competent cells was mixed with 400 ng of the PCR fragment in an ice-cold 0.1 cm cuvette (Bio-Rad Inc., Hercules, CA, USA). Cells were electroporated at 1.8 kV with 25 mF and 200, immediately followed by the addition of 1 ml of SOC medium (2% Bacto Tryptone (Difco), 0.5% yeast extract (Difco), 10 mM NaCl, 2.5 mM KCl, 10 mM MgCl₂, 10 mM MgSO₄, 20 mM glucose) with 1 mM L-arabinose. After incubation for 2 h at 37° C., one-tenth portion was spread onto agar plate to select chloramphenicolR or kanamycinR transformants at 37° C. The individual deletions were then transduced into new host strains using P1vir and selecting for the appropriate antibiotic (Silhavy et al., Experiments with gene fusions, Cold Spring Harbor Laboratory, Cold Spring Harbor, NY (1984)). The chloramphenicol or kanamycin resistant colonies were then transformed with the temperature sensitive plasmid pCP20, and plated on ampicillin (100 μg/ml) plates at 30° C. pCP20 contains the yeast Flp recombinase gene, chloramphenicol and ampicillin resistant genes, and temperature sensitive replication. Colonies were grown in LB medium without antibiotics at 42° C. overnight and streak plated on prewarmed LB Agar plates and grown at 42° C. Individual clones were tested for sensitivity to ampicillin and chloramphenicol or kanamycin to isolate clones which had lost the pCP20 plasmid and had the chloramphenicol or kanamycin resistance gene lost via the Flp recombinase. Positive clones with deletions in cyoABCD contain a single FRT “scar” and were verified by PCR using primers which flanked their respective genes.

The nucleotides deleted because of the cyoABCD deletion were: 449,887->447,270

The esterase from Yersinia intermedia was integrated in the chromosome with the standard pRed technique described above. The primers used for making the insertion were: (5′-3′)

| | | SEQ |
|---|---|---|
| Primer | | ID |
| Designation | Primer Sequence | NO |
| JE310 | ACGCTATGGAACTGCAGGATGAGAGCATGAAA | 155 |
| | TGGAACGGATGCGCGCCTTAT | |
| JE311 | ACGCTATGGAACTGCAGGATGAGAGCATCTGC | 156 |
| | CCCAGATCGTTGGCTTTTTGC | |
| JE312 | TTTGAAAACAGGATGTAGCGATGACCGATATT | 157 |
| | CGCTTGTA | |
| JE312-rv | TACAAGCGAATATCGGTCATCGCTACATCCTG | 158 |
| | TTTTCAAA | |
| JE313 | CGCCTGAATACTACGATTGACTGGAGAATGCG | 159 |
| | AATGAACA | |
| JE313-rv | TGTTCATTCGCATTCTCCAGTCAATCGTAGT | 160 |
| | ATTCAGGCG | |
| JE314 | TTTGAAAACAGGATGTAGCGGCCCGTAGTCT | 161 |
| | GCAAATCC | |
| JE314-rv | GGATTTGCAGACTACGGGCCGCTACATCCTG | 162 |
| | TTTTCAAA | |
| JE315 | GCATTACGCTGACTTGACGGCTGGAGAATGC | 163 |
| | GAATGAACA | |
| JE315-rv | TGTTCATTCGCATTCTCCAGCCGTCAAGTCA | 164 |
| | GCGTAATGC | |

The sequence that was inserted at the gabD locus (ORF swap) was:

(SEQ ID NO: 165)
ACTGGAGAATGCGAATGAACAGCAATAAAGAGTTAATGCAGCGCCGCAG
TCAGGCGATTCCCCGTGGCGTTGGGCAAATTCACCCGATTTTCGCTGAC
CGCGCGGAAAACTGCCGGGTGTGGGACGTTGAAGGCCGTGAGTATCTTG
ATTTCGCGGGCGGGATTGCGGTGCTCAATACCGGGCACCTGCATCCGAA
GGTG.

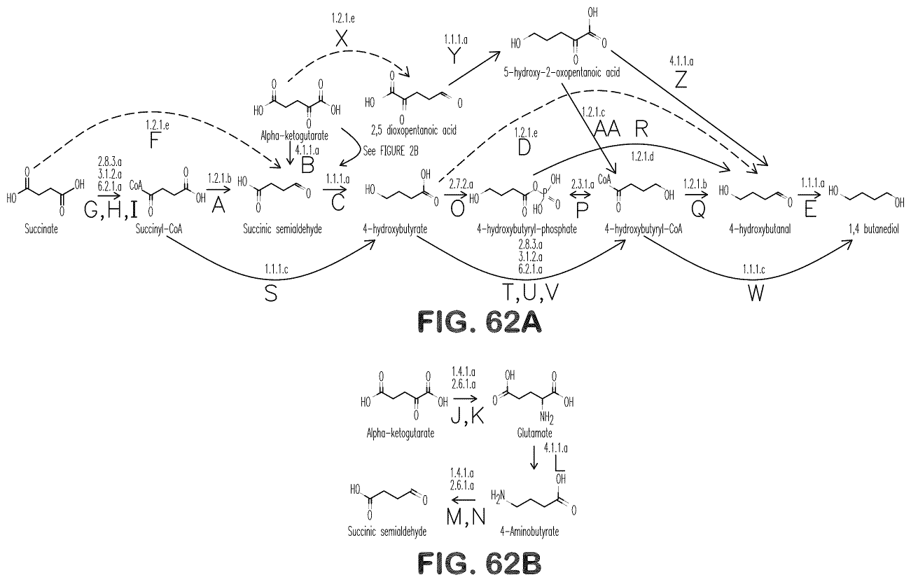

Markerless deletion of sucC and sucD. Deletion of the native Escherichia coli sucC (gene 2) and sucD (gene 3) from the chromosome (FIG. 67) was achieved using a two-step double-crossover homologous recombination method. Recombination was catalyzed via expression of the Lambda phage Red genes from pRED-Amp (Gene Bridges, Heidelberg, Germany). Genes encoding levansucrase (gene 6) and kanamycin resistance (gene 7) were integrated into the chromosome, replacing the majority of sucC (gene 2) and sucD (gene 3). Kanamycin was used to select for successful integrants. In a second double-crossover homologous recombination step, the integrated sequence containing the levansucrase gene (gene 6) and kanamycin resistance gene (gene 7) was replaced with an appropriate DNA sequence lacking the majority of sucC (gene 2) and sucD (gene 3), and sucrose resistant clones were selected for.

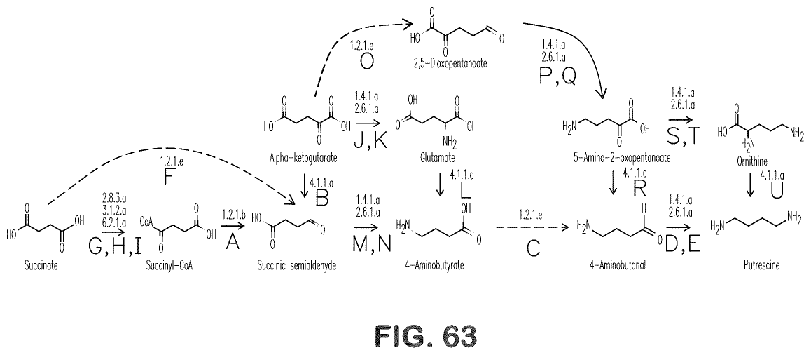

Linear double-stranded DNA sequences used in the two homologous recombination steps outlined above were constructed via standard molecular biological techniques. To make the sequence used in the initial integration step, a 1100 bp sequence from within sucB (gene 1) through the 69th base pair of sucC (gene 2) was amplified by PCR from Escherichia coli MG1655 genomic DNA using oligonucleotide 1 and oligonucleotide 3 (FIG. 68a). The genes encoding levansucrase (gene 6) and kanamycin resistance (gene 7) were amplified by PCR from plasmid pRE-118 DNA using oligonucleotide 5 and oligonucleotide 6 (FIG. 68b). Another 1100 bp sequence from the 811th bp of sucD (gene 3), spanning mngR (gene 4) and part of mngA (gene 5) was amplified by PCR from Escherichia coli MG1655 genomic DNA using oligonucleotide 4 and oligonucleotide 2 (FIG. 68a). 20 bp of oligonucleotide 3 overlaps with oligonucleotide 5 and 20 bp of oligonucleotide 6 overlaps with oligonucleotide 4, such that the three overlapping PCR products can be combined and amplified using standard procedures (FIG. 68c). Likewise, the linear double-stranded DNA sequences used in the second recombination for deletion of sucCD was made by combining and amplifying two overlapping PCR products. A 1100 bp sequence from within sucB (gene 1) through the 69th base pair of sucC (gene 2) was amplified by PCR from Escherichia coli MG1655 genomic DNA using oligonucleotide 1 and oligonucleotide 8 (FIG. 68d). Another 1100 bp sequence from the 811th bp of sucD (gene 3), spanning mngR (gene 4) and part of mngA (gene 5) was amplified by PCR from Escherichia coli MG1655 genomic DNA using oligonucleotide 7 and oligonucleotide 2 (FIG. 68d). Oligonucleotide 7 overlaps with oligonucleotide 8, such that the two overlapping PCR products can be combined and amplified using standard procedures (FIG. 68e). The nucleotides deleted because of the sucCD deletion are from: 762,306->

| | | SEQ |
|---|---|---|
| Oligo- | | ID |
| nucleotide | | NO |
| 1 | GGCGAAAGAGTCTGCTCCGGC | 166 |
| 2 | CATTCAGACACAACGCATCGCGG | 167 |
| 3 | GGCTTACCAGCACCGGTGGGTTATGCCCGTAG | 168 |
| | TCTGCAAATCC | |
| 4 | GCATTACGCTGACTTGACGGGTGAAAACCGTT | 169 |
| | CGCAGCCTGG | |
| 5 | GGCTTACCAGCACCGGTGGGTTATGCCCGTAG | 170 |
| | TCTGCAAATCC | |
| 6 | CCAGGCTGCGAACGGTTTTCACCCGTCAAGTC | 171 |
| | AGCGTAATGC | |
| 7 | GGCTTACCAGCACCGGTGGGTTATGTGAAAAC | 172 |
| | CGTTCGCAGCCTGG | |
| 8 | CCAGGCTGCGAACGGTTTTCACATAACCCACC | 173 |
| | GGTGCTGGTAAGCC | |

Analytical Procedures: HPLC, LCMS/MS and GCMS were employed for analysis of cell culture and fermentation samples. Good correlation between the two techniques (variation within 10%) provided method cross-validation and ensured data accuracy.

Organic acids, acetate, pyruvate, lactate and succinate, as well as BDO, were measured by HPLC (Agilent 1100) equipped with diode array and refractive index (RI) detectors, using an ion exclusion Carbomix H-NP5 column (Sepax) with 5 mM sulfuric acid as the mobile phase at 0.6 mL/min flow rate, and 55° C. column temperature. Organic acids were monitored in both UV absorption at 210 nm and refractive index detector, while BDO, ethanol and simple sugars were measured using RI only. All chemicals and reagents were from Sigma-Aldrich (St. Louis, MO, USA).

LCMS/MS analysis was performed on API3200 triple quadrupole system (AB Sciex, Life Technologies, Carlsbad, CA), interfaced with Agilent 1200 HPLC, utilizing electrospray ionization and MRM based acquisition methods. BDO, 4HB, GBL, Glu, Ala and GABA and internal standards (¹³C isotopically labeled analogs were used for each analyte, respectively, to compensate for the matrix effects and ensure linearity) were monitored in positive ionization mode. Negative ionization was used, when desired, for quantitation of acidic analytes, for example, pyruvate, lactate, succinate and 4HB. Chromatographic separation was conducted on Zorbax Eclipse XDB C18 4.6×30 mm, 1.8 μm particle size, maintaining column at 45° C., flow rate 0.7 mL/min. Injection volume was 5 uL. Eluents consisted of water with 0.1% formic acid and methanol with 0.1% formic acid, analytes of interest eluted under isocratic conditions of 5% methanol followed by the step gradient to 95% methanol, resulting in 2 min long LCMS method. Filtered samples were diluted in water, containing internal standards, dilution factor varied from 200 to 10,000 depending on concentrations of metabolites of interest. Dilution in water of 100-fold of greater resulted in no ion suppression effects observed for media composition and conditions used in 1,4-Butanediol production in E. coli (Yim et al., Nature Chemical Biology 7, 445-452, (2011)). Quantitation dynamic ranges were from 0.05-200 uM for BDO and other analytes. Quantitation was performed using Analyst software. LCMS/MS approach was a preferred method for monitoring BDO, 4HB, GBL, Glu, Ala and GABA due to its fast turnaround time allowing high throughput operation.

In GCMS approach, BDO, 4HB, lactate and succinate in culture broth samples were derivatized by trimethylsilylation and quantitatively analyzed by GCMS, using a procedure adapted from the literature with some modifications. The developed method demonstrated sensitivity down to low M level, linearity in 0.05-15 mM range, as well as good reproducibility. 100 μL filtered samples were dried down in a speedvac concentrator for approximately 1 hour at ambient temperature, followed by the addition of 20 μL 10 mM cyclohexanol solution, as an internal standard, in dimethylformamide. 100 μL N,O-bis(trimethylsilyl)triflouro-acetimide (BSTFA) with 1% trimethylchlorosilane was added, and the mixture was incubated at 70° C. for 30 min. The derivatized samples were centrifuged for 5 min and directly injected into GCMS (Agilent 6890N/5973N). An Rtx-5SIL MS capillary column (Restek, 30 m×0.25 mm ID×0.25 μm film thickness) was used. The GC was operated in a split injection mode introducing 1 μL of sample at 20:1 split ratio. The injection port temperature was 250° C. Helium was used as a carrier gas, and the flow rate was maintained at 1.0 mL/min. A temperature gradient program was optimized to ensure good resolution of the analytes of interest and minimum matrix interference. The oven was initially held at 80° C. for 1.5 min, then ramped to 140° C. at 10° C./min and 3 min hold, followed by fast ramping to 300° C. at 100° C./min and final hold for 5 min. The MS interface transfer line was maintained at 280° C. The total analysis time was 17 min including 4 min solvent delay. Standard calibration curves were constructed using analyte solutions in the corresponding cell culture or fermentation medium to match sample matrix as close as possible. Ethanol was assayed by GCMS as well, following sample dilution in methanol, using an HP-InnoWax capillary column (30 m, 0.25 mm ID, 0.25 um film thickness). GCMS data were processed using ChemStation software (Agilent).

Residual glucose in the fermentation samples was measured using either a YSI instrument or HPLC-UV-RI. Ethanol was measured by either YSI or GCMS.

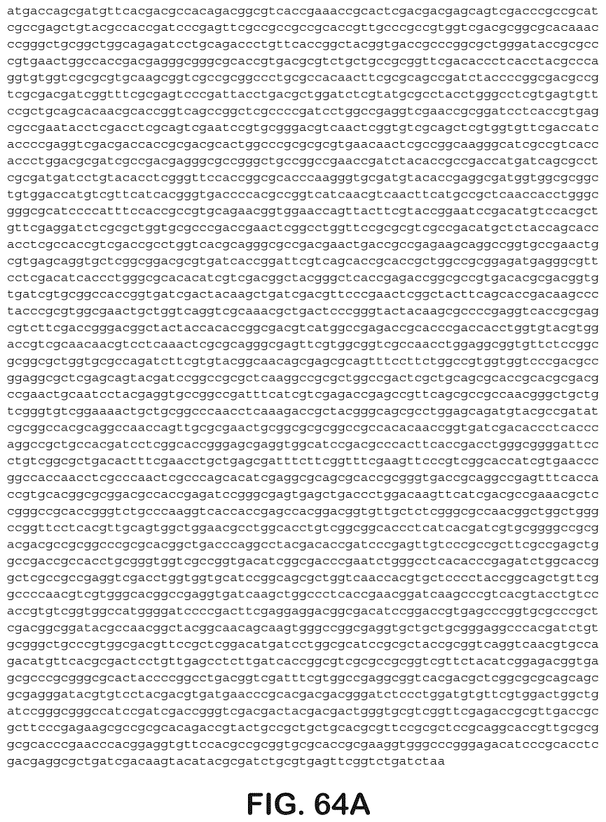

The metabolic engineering strategies described herein were applied to increase 1,4-butanediol production in Escherichia coli or otherwise improve characteristics of the engineered microorganisms. The employed 1,4-butanediol pathway for these studies is depicted in FIG. 69. The genes tested for each of the six steps shown in FIG. 69 are described in Tables 22, 26, 28, 30, 34 and 37.

### Example XXV

### Production of 4-Hydroxybutyrate and 1,4-Butanediol Upon Expression of Genes Encoding Alpha-Ketoglutarate Decarboxylase Enzymes

This example describes production of 4-hydroxybutyrate (4HB or 4hb) and 1,4-butanediol (BDO) in cells engineered to express genes encoding alpha-ketoglutarate decarboxylase enzymes. See corresponding example in WO2013/186402.

Several heterologous alpha-ketoglutarate decarboxylase genes (Table 22) were expressed in E. coli from the pZS*-13S plasmid to allow a functional pathway from alpha-ketoglutarate to 4-hydroxybutyrate and 1,4-butanediol. Host strains include 3125, 2869, and 3812. These host strains are based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). For this study, the M. bovis sucA and P. gingivalis sucD genes were deleted in strains 3125, 2869, and 3812. Additionally, strains 3125,2869, and 3812 each contain a chromosomally integrated copy of 4-hydroxybutyryl-CoA transferase expressed from the pA promoter at the attB locus. Strains 2869 and 3812 were tested with 4-hydroxybutyryl-CoA reductase and 4-hydroxybutyraldehyde reductase expressed from the pPSX23R plasmid. Tables 23, 24, and 25 show that expression of heterologous alpha-ketoglutarate decarboxylase genes enhances 4hb and/or BDO production in strains 3125, 2869, and 3812, respectively, over the controls denoted by “pZS*13 S”. Cells were cultured using the 96 well-plate protocol described previously.

TABLE 22
Genes encoding functional alpha-ketoglutarate
decarboxylase enzymes.

| Gene | | | |
|---|---|---|---|
| Number | Organism | Accession | GI |
| 42 | Mycobacterium tuberculosis | ZP_06504371.1 | 289744993 |
| 1717 | Mycobacterium smegmatis | YP_889299.1 | 118472055 |
| 1720 | Saccharopolyspora erythraea | YP_001108480.1 | 134102819 |
| 1727 | Jonesia denitrificans | YP_003161752.1 | 256833025 |
| 1715 | Mycobacterium avium subsp. | NP_961470.1 | 41408634 |
| | Paratuberculosis | | |
| 1718 | Nocardia farcinica | YP_120910.2 | 161598437 |
| 1726 | Streptomyces avermitilis | NP_824148.1 | 29829514 |
| 1727 | Jonesia denitrificans | YP_003161752.1 | 256833025 |
| 1913 | Zymomonas mobilis | YP_162422.1 | 56551583 |
| 1914 | Pseudomonas putida | NP_743318.1 | 26987893 |
| 1915 | Mycobacterium avium subsp. | EGO39007.1 | 336460099 |
| | Paratuberculosis | | |
| 1916 | Streptomyces griseus | YP_001827718.1 | 182439999 |
| 1917 | Methanosarcina acetivorans | NP_616881.1 | 20090806 |
| 1918 | Clostridium ljungdahlii | YP_003782019.1 | 300857035 |
| 1920 | Synechocystis sp. PCC 6803 | NP_441304.1 | 16330576 |
| 1921 | Synechococcus sp. PCC 7335 | ZP_05039761.1 | 254426044 |
| 1922 | Clostridium acetobutylicum | NP_350234.1 | 15896885 |
| 1923 | Clostridium carboxidivorans | ZP_05391417.1 | 255524461 |
| | P7 | | |

TABLE 23
BDO and 4-hydroxybutyrate (4HB) production of E. coli host
strain 3125 containing several alternative alpha-ketoglutarate
decarboxylase plasmid constructs. The plasmid construct denoted
by only “pZS*13S” contains no alpha-ketoglutarate decarboxylase
gene. Plasmid constructs denoted by pZS*13S followed by a number
express the alpha-ketoglutarate decarboxylase gene denoted by
such number. BDO and 4HB concentrations are in mM.

| Host | | | LCMS-19 h | LCMS-19 h |
|---|---|---|---|---|
| strain | Plasmid | OD 19 h | 4HB | BDO |
| 3125 | pZS*13S | 3.61 | 0.208 | 0.327 |
| 3125 | pZS*13S | 2.01 | 0.259 | 0.082 |
| 3125 | pZS*13S | 3.39 | 0.208 | 0.359 |
| 3125 | pZS*13S | 3.38 | 0.178 | 0.352 |
| 3125 | pZS*13S | 3.84 | 0.277 | 0.478 |
| 3125 | pZS*13S | 4.01 | 0.253 | 0.479 |
| 3125 | pZS*13S | 3.88 | 0.283 | 0.422 |
| 3125 | pZS*13S | 3.54 | 0.192 | 0.499 |
| 3125 | pZS*13S-1717 | 4.30 | 17.300 | 1.520 |
| 3125 | pZS*13S-1717 | 3.52 | 17.400 | 1.280 |
| 3125 | pZS*13S-1717 | 3.79 | 17.700 | 1.540 |
| 3125 | pZS*13S-1717 | 3.53 | 17.800 | 1.550 |
| 3125 | pZS*13S-1720 | 2.92 | 3.940 | 0.196 |
| 3125 | pZS*13S-1720 | 1.58 | 2.140 | 0.037 |
| 3125 | pZS*13S-1720 | 1.97 | 2.600 | 0.072 |
| 3125 | pZS*13S-1720 | 3.11 | 3.870 | 0.261 |
| 3125 | pZS*13S-1727 | 12.05 | 2.330 | 0.694 |
| 3125 | pZS*13S-1727 | 3.23 | 3.040 | 1.320 |
| 3125 | pZS*13S-1727 | 3.19 | 3.490 | 1.560 |
| 3125 | pZS*13S-1727 | 4.86 | 2.500 | 0.657 |
| 3125 | pZS*13S-1715 | 3.89 | 14.900 | 1.880 |
| 3125 | pZS*13S-1715 | 4.14 | 15.600 | 1.950 |
| 3125 | pZS*13S-1715 | 4.07 | 15.700 | 1.910 |
| 3125 | pZS*13S-1715 | 3.03 | 14.300 | 1.600 |
| 3125 | pZS*13S-1718 | 2.36 | 4.020 | 0.277 |
| 3125 | pZS*13S-1718 | 1.82 | 4.630 | 0.406 |
| 3125 | pZS*13S-1718 | 2.37 | 3.690 | 0.265 |
| 3125 | pZS*13S-1718 | 2.26 | 3.940 | 0.273 |
| 3125 | pZS*13S-1718 | 2.04 | 4.890 | 0.389 |
| 3125 | pZS*13S-1718 | 2.42 | 4.720 | 0.338 |
| 3125 | pZS*13S-1718 | 2.16 | 4.530 | 0.377 |
| 3125 | pZS*13S-1718 | 2.43 | 5.190 | 0.517 |

TABLE 24
BDO and 4-hydroxybutyrate (4HB) production of E. coli host
strain 2869 containing several alternative alpha-ketoglutarate
decarboxylase plasmid constructs. The plasmid construct denoted
by only “pZS*13S” contains no alpha-ketoglutarate decarboxylase
gene. Plasmid constructs denoted by pZS*13S followed by a number
express the alpha-ketoglutarate decarboxylase gene denoted by
such number. BDO and 4HB concentrations are in mM.

| Host | | | LCMS-16 h | LCMS-16 h |
|---|---|---|---|---|
| strain | Plasmid | OD 16 h | 4HB | BDO |
| 2869 | pZS*13S | 3.39 | 0.04 | 0.42 |
| 2869 | pZS*13S | 3.41 | 0.04 | 0.42 |
| 2869 | pZS*13S | 3.43 | 0.04 | 0.35 |
| 2869 | pZS*13S | 3.51 | 0.04 | 0.39 |
| 2869 | pZS*13S | 3.51 | 0.03 | 0.39 |
| 2869 | pZS*13S | 3.50 | 0.04 | 0.40 |
| 2869 | pZS*13S | 3.55 | 0.03 | 0.36 |
| 2869 | pZS*13S | 3.50 | 0.02 | 0.35 |
| 2869 | pZS*13S-042 | 3.50 | 0.33 | 2.81 |
| 2869 | pZS*13S-042 | 3.52 | 0.32 | 2.69 |
| 2869 | pZS*13S-042 | 3.58 | 0.32 | 3.05 |
| 2869 | pZS*13S-042 | 3.49 | 0.28 | 2.61 |
| 2869 | pZS*13S-042 | 3.39 | 0.30 | 2.87 |
| 2869 | pZS*13S-042 | 3.41 | 0.28 | 3.06 |
| 2869 | pZS*13S-042 | 3.44 | 0.27 | 2.64 |
| 2869 | pZS*13S-042 | 3.28 | 0.33 | 2.90 |
| 2869 | pZS*13S-1726 | 2.74 | 0.34 | 2.27 |
| 2869 | pZS*13S-1726 | 2.78 | 0.23 | 2.75 |
| 2869 | pZS*13S-1726 | 2.96 | 0.43 | 2.80 |
| 2869 | pZS*13S-1726 | 2.84 | 0.34 | 2.33 |
| 2869 | pZS*13S-1727 | 3.43 | 0.06 | 2.39 |
| 2869 | pZS*13S-1727 | 2.79 | 0.07 | 2.18 |
| 2869 | pZS*13S-1727 | 3.09 | 0.07 | 2.12 |
| 2869 | pZS*13S-1727 | 3.03 | 0.07 | 2.38 |

TABLE 25
BDO and 4-hydroxybutyrate (4HB) production of E. coli host
strain 3812 containing several alternative alpha-ketoglutarate
decarboxylase plasmid constructs. The plasmid construct denoted
by only “pZS*13S” contains no alpha-ketoglutarate decarboxylase
gene. Plasmid constructs denoted by pZS*13S followed by a number
express the alpha-ketoglutarate decarboxylase gene denoted by
such number. BDO and 4HB concentrations are in mM.

| Host | | | LCMS-24 h | LCMS-24 h |
|---|---|---|---|---|
| strain | Plasmid | OD 24 h | 4HB | BDO |
| 3812 | pZS*13S | 3.75 | 0.07 | 1.71 |
| 3812 | pZS*13S | 3.74 | 0.05 | 1.46 |
| 3812 | pZS*13S | 3.49 | 0.04 | 2.08 |
| 3812 | pZS*13S | 3.59 | 0.05 | 2.31 |
| 3812 | pZS*13S-1913 | 3.40 | 6.51 | 9.25 |
| 3812 | pZS*13S-1913 | 3.28 | 6.46 | 8.69 |
| 3812 | pZS*13S-1913 | 3.41 | 6.22 | 8.53 |
| 3812 | pZS*13S-1913 | 3.25 | 5.94 | 7.89 |
| 3812 | pZS*13S-1914 | 3.83 | 23.10 | 85.00 |
| 3812 | pZS*13S-1914 | 3.41 | 29.70 | 93.10 |
| 3812 | pZS*13S-1914 | 3.68 | 19.90 | 63.10 |
| 3812 | pZS*13S-1914 | 3.45 | 27.20 | 101.00 |
| 3812 | pZS*13S-1915 | 4.17 | 0.30 | 3.28 |
| 3812 | pZS*13S-1915 | 3.78 | 0.18 | 3.11 |
| 3812 | pZS*13S-1915 | 3.95 | 0.14 | 3.08 |
| 3812 | pZS*13S-1915 | 3.97 | 0.11 | 2.80 |
| 3812 | pZS*13S-1916 | 4.09 | 0.07 | 4.82 |
| 3812 | pZS*13S-1916 | 3.69 | 0.12 | 4.54 |
| 3812 | pZS*13S-1916 | 3.86 | 0.09 | 4.73 |
| 3812 | pZS*13S-1916 | 3.82 | 0.08 | 4.89 |
| 3812 | pZS*13S-1917 | 3.61 | 0.08 | 2.70 |
| 3812 | pZS*13S-1917 | 3.99 | 0.06 | 2.92 |
| 3812 | pZS*13S-1917 | 3.59 | 0.12 | 2.74 |
| 3812 | pZS*13S-1917 | 3.57 | 0.11 | 2.64 |
| 3812 | pZS*13S-1918 | 3.10 | 0.25 | 6.43 |
| 3812 | pZS*13S-1918 | 2.93 | 0.34 | 5.94 |
| 3812 | pZS*13S-1918 | 2.98 | 0.23 | 6.48 |
| 3812 | pZS*13S-1918 | 3.03 | 0.23 | 6.53 |
| 3812 | pZS*13S-1920 | 3.30 | 11.20 | 44.10 |
| 3812 | pZS*13S-1920 | 3.57 | 16.60 | 64.20 |
| 3812 | pZS*13S-1920 | 3.71 | 15.00 | 59.10 |
| 3812 | pZS*13S-1920 | 3.55 | 16.30 | 62.40 |
| 3812 | pZS*13S-1921 | 3.99 | 0.23 | 9.07 |
| 3812 | pZS*13S-1921 | 3.70 | 0.24 | 8.76 |
| 3812 | pZS*13S-1921 | 4.01 | 0.24 | 9.79 |
| 3812 | pZS*13S-1921 | 3.88 | 0.27 | 9.35 |
| 3812 | pZS*13S-1922 | 2.46 | 0.06 | 4.00 |
| 3812 | pZS*13S-1922 | 2.79 | 0.23 | 5.55 |
| 3812 | pZS*13S-1922 | 2.61 | 0.23 | 5.25 |
| 3812 | pZS*13S-1922 | 2.93 | 0.18 | 5.53 |
| 3812 | pZS*13S-1923 | 2.82 | 0.25 | 6.46 |
| 3812 | pZS*13S-1923 | 2.48 | 0.22 | 6.43 |
| 3812 | pZS*13S-1923 | 2.69 | 0.17 | 4.42 |
| 3812 | pZS*13S-1923 | 3.18 | 0.18 | 10.50 |

### EXAMPLE XXVI

### Production of 4HB and BDO Upon Expression of Genes Encoding CoA-Dependant Succinate Semialdehyde Dehydrogenase Enzymes

This example describes production of 4 TB and BDO upon expression of genes encoding CoA-dependant succinate semialdehyde dehydrogenase enzymes. See corresponding example in WO2013/186402.

Several heterologous CoA-dependant succinate semialdehyde dehydrogenase genes (Table 26) were expressed in E. coli from the pZS*-13S plasmid to allow afunctional pathway from succinyl-CoA to 4-hydroxybutyrate and 1,4-butanediol. Host strain 3744 is based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). For this study, the M. bovis sucA and P. gingivalis sucD genes were deleted in strain 3744. Additionally, strain 3744 contains a chromosomally integrated copy of 4-hydroxybutyryl-CoA transferase expressed from the pA promoter at the attB locus. It also contains a pPSX23R plasmid expressing 4-hydroxybutyryl-CoA reductase and 4-hydroxybutyraldehyde reductase. Table 27 shows that expression of heterologous CoA-dependant succinate semialdehyde dehydrogenase genes enhances 4hb and/or BDO production in strain 3744 over the controls denoted by “pZS*13 S”. Cells were cultured using the 96 well-plate protocol described previously.

TABLE 26
Genes Encoding Functional CoA-dependant Succinate
Semialdehyde Dehydrogenase Enzymes

| Gene | | | |
|---|---|---|---|
| Number | Organism | Accession | GI |
| 35 | Porphyromonas gingivalis | NP_904963.1 | 34540484 |
| | W83 | | |
| 49 | Clostridium difficile 630 | YP_001088857.1 | 126699960 |
| 1870 | Metallosphaera sedula | YP_001190808.1 | 146303492 |
| 1871 | Porphyromonas endodontalis | ZP_04389701.1 | 229495977 |
| | ATCC 35406 | | |
| 1872 | Clostridium sporogenes | ZP_02995751.1 | 187779278 |
| 1931 | Porphyromonas | ZP_07821123.1 | 313887434 |
| | asaccharolytica DSM 20707 | | |
| 1932 | Odoribacter splanchnicus | YP_004253242.1 | 325280700 |
| | DSM 20712 | | |

TABLE 27
BDO and 4-hydroxybutyrate (4HB) production of E. coli host
strain 3744 containing several alternative CoA-dependant succinate
semialdehyde dehydrogenase plasmid constructs. The plasmid construct
denoted by only “pZS*13S” contains no CoA-dependant
succinate semialdehyde dehydrogenase gene. Plasmid constructs
denoted by pZS*13S followed by a number express the CoA-dependant
succinate semialdehyde dehydrogenase gene denoted by such number.
BDO and 4HB concentrations are in mM.

| Host | | | LCMS-16 h | LCMS-16 h |
|---|---|---|---|---|
| strain | Plasmid | OD-16 h | 4HB | BDO |
| 3744 | pZS*13S | 3.10 | 0.47 | 3.08 |
| 3744 | pZS*13S | 2.97 | 0.54 | 2.69 |
| 3744 | pZS*13S | 3.18 | 0.89 | 4.09 |
| 3744 | pZS*13S | 1.26 | 0.44 | 2.41 |
| 3744 | pZS*13S-035 | 3.63 | 7.29 | 30.80 |
| 3744 | pZS*13S-035 | 2.89 | 6.86 | 28.30 |
| 3744 | pZS*13S-035 | 3.22 | 6.64 | 28.40 |
| 3744 | pZS*13S-035 | 3.73 | 7.55 | 28.80 |
| 3744 | pZS*13S-049 | 1.87 | 10.50 | 23.30 |
| 3744 | pZS*13S-049 | 1.65 | 10.10 | 23.70 |
| 3744 | pZS*13S-049 | 1.87 | 10.80 | 23.60 |
| 3744 | pZS*13S-049 | 1.75 | 11.40 | 23.70 |
| 3744 | pZS*13S-1870 | 3.58 | 0.43 | 7.18 |
| 3744 | pZS*13S-1870 | 3.51 | 0.57 | 7.28 |
| 3744 | pZS*13S-1870 | 3.15 | 0.50 | 5.59 |
| 3744 | pZS*13S-1870 | 2.98 | 0.44 | 6.78 |
| 3744 | pZS*13S-1871 | 3.33 | 6.73 | 37.20 |
| 3744 | pZS*13S-1871 | 3.28 | 6.42 | 31.90 |
| 3744 | pZS*13S-1871 | 3.20 | 7.18 | 32.40 |
| 3744 | pZS*13S-1871 | 3.44 | 7.03 | 33.90 |
| 3744 | pZS*13S-1872 | 3.52 | 0.44 | 4.52 |
| 3744 | pZS*13S-1872 | 3.54 | 0.49 | 4.71 |
| 3744 | pZS*13S-1872 | 3.56 | 0.38 | 4.51 |
| 3744 | pZS*13S-1872 | 3.37 | 0.37 | 4.49 |
| 3744 | pZS*13S-1872 | 3.47 | 0.50 | 4.32 |
| 3744 | pZS*13S-1872 | 3.58 | 0.56 | 4.68 |
| 3744 | pZS*13S-1872 | 3.47 | 0.49 | 4.27 |
| 3744 | pZS*13S-1872 | 3.90 | 0.43 | 4.51 |
| | | OD-24 hr | LCMS-24 h | LCMS-24 h |
| 3744 | pZS*13S | 3.89 | 0.12 | 8.85 |
| 3744 | pZS*13S | 3.79 | 0.17 | 8.57 |
| 3744 | pZS*13S | 3.85 | 0.17 | 8.87 |
| 3744 | pZS*13S | 3.84 | 0.12 | 9.05 |
| 3744 | pZS*13S-1931 | 3.78 | 3.18 | 45.10 |
| 3744 | pZS*13S-1931 | 3.64 | 3.28 | 46.50 |
| 3744 | pZS*13S-1931 | 2.91 | 4.25 | 34.00 |
| 3744 | pZS*13S-1931 | 3.73 | 3.22 | 45.40 |
| 3744 | pZS*13S-1932 | 3.71 | 3.10 | 84.20 |
| 3744 | pZS*13S-1932 | 3.63 | 3.24 | 79.50 |
| 3744 | pZS*13S-1932 | 3.61 | 3.18 | 82.60 |
| 3744 | pZS*13S-1932 | 3.61 | 3.24 | 83.90 |

### Example XXVII

### Production of 41113 and BDO Upon Expression of Genes Encoding 4-Hydroxybutyrate Dehydrogenase Enzymes

This example describes production of 4 TB and BDO upon expression of genes encoding 4-hydroxybutyrate dehydrogenase enzymes.

Several heterologous 4-hydroxybutyrate dehydrogenase genes (Table 28) were expressed in E. coli from the pZS*-13S plasmid to allow afunctional pathway from succinyl-CoA and alpha-ketoglutarate to 4-hydroxybutyrate and 1,4-butanediol. Host strain 3891 is based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). For this study, the P. gingivalis and C. Kluyveri 4hbd genes were deleted in strain 3891. Additionally, strain 3891 contains a chromosomally integrated copy of 4-hydroxybutyryl-CoA transferase expressed from the pA promoter at the attB locus. It also contains a pPSX23R plasmid expressing 4-hydroxybutyryl-CoA reductase and 4-hydroxybutyraldehyde reductase. Table 29 shows that expression of heterologous 4-hydroxybutyrate dehydrogenase genes enhances 4hb and/or BDO production in strain 3891 over the controls denoted by “pZS*13 S”. Cells were cultured using the 96 well-plate protocol described previously.

TABLE 28
Genes encoding functional 4-hydroxybutyrate
dehydrogenase enzymes.

| Gene | | | |
|---|---|---|---|
| Number | Organism | Accession | GI |
| 36 | Porphyromonas gingivalis | NP_904964.1 | 34540485 |
| | W83 | | |
| 150 | Clostridium acetobutylicum | NP_348201.1 | 15894852 |
| | ATCC 824 | | |
| 1879 | Porphyromonas endodontalis | ZP_04389726.1 | 229496002 |
| | ATCC 35406 | | |
| 1880 | Porphyromonas | YP_004441566.1 | 332299645 |
| | asaccharolytica DSM 20707 | | |
| 1881 | Odoribacter splanchnicus | YP_004253243.1 | 325280701 |
| | DSM 20712 | | |
| 1882 | Eubacterium saphenum | ZP_05427218.1 | 255994083 |
| | ATCC 49989 | | |
| 1883 | Clostridium ljungdahlii DSM | YP_003782020.1 | 300857036 |
| | 13528 | | |
| 1884 | Clostridium perfringens | YP_694972.1 | 110800333 |
| | ATCC 13124 | | |
| 1888 | Clostridium difficile 630 | YP_001088853.1 | 126699956 |
| 1889 | Geobacter sulferreducens | NP_952425.1 | 39996474 |
| 1894 | Metallosphaera sedula DSM | YP_001191506.1 | 146304190 |
| | 5348 (ATCC 51363D-5) | | |

TABLE 29
BDO and 4-hydroxybutyrate (4HB) production of E. coli host strain 3891 containing several
alternative 4-hydroxybutyrate dehydrogenase plasmid constructs. The plasmid construct denoted by only
“pZS*13S” contains no 4-hydroxybutyrate dehydrogenase gene. Plasmid constructs denoted by pZS*13S
followed by a number express the 4-hydroxybutyrate dehydrogenase gene denoted by such number. BDO
and 4HB concentrations are in mM.

| | | | LCMS-16 h | LCMS-16 h |
|---|---|---|---|---|
| Host | Plasmid | OD-16 h | 4HB | BDO |
| 3891 | pZS*13S | 3.26 | 0.07 | 1.33 |
| 3891 | pZS*13S | 3.06 | 0.04 | 1.38 |
| 3891 | pZS*13S | 3.17 | 0.04 | 1.80 |
| 3891 | pZS*13S | 3.22 | 0.04 | 1.25 |
| 3891 | pZS*13S-036 | 3.11 | 7.19 | 15.90 |
| 3891 | pZS*13S-036 | 2.95 | 7.31 | 17.40 |
| 3891 | pZS*13S-036 | 3.17 | 7.00 | 16.40 |
| 3891 | pZS*13S-036 | 2.80 | 6.84 | 16.60 |
| 3891 | pZS*13S-150 | 2.99 | 2.58 | 31.20 |
| 3891 | pZS*13S-150 | 3.41 | 2.49 | 33.30 |
| 3891 | pZS*13S-150 | 3.11 | 2.88 | 32.40 |
| 3891 | pZS*13S-150 | 2.91 | 2.71 | 33.30 |
| 3891 | pZS*13S-1879 | 3.35 | 1.99 | 9.78 |
| 3891 | pZS*13S-1879 | 3.12 | 2.26 | 10.80 |
| 3891 | pZS*13S-1879 | 3.42 | 2.06 | 9.58 |
| 3891 | pZS*13S-1879 | 3.16 | 2.21 | 10.20 |
| 3891 | pZS*13S-1880 | 2.01 | 3.99 | 13.30 |
| 3891 | pZS*13S-1880 | 2.85 | 4.49 | 14.00 |
| 3891 | pZS*13S-1880 | 2.99 | 4.52 | 12.90 |
| 3891 | pZS*13S-1880 | 2.95 | 4.04 | 12.70 |
| 3891 | pZS*13S-1881 | 3.18 | 3.73 | 7.00 |
| 3891 | pZS*13S-1881 | 2.95 | 3.56 | 7.64 |
| 3891 | pZS*13S-1881 | 3.03 | 3.40 | 6.98 |
| 3891 | pZS*13S-1881 | 2.96 | 3.68 | 7.50 |
| 3891 | pZS*13S-1882 | 3.20 | 3.62 | 22.70 |
| 3891 | pZS*13S-1882 | 3.13 | 3.85 | 24.10 |
| 3891 | pZS*13S-1882 | 3.33 | 3.34 | 22.20 |
| 3891 | pZS*13S-1882 | 2.60 | 3.67 | 19.80 |
| 3891 | pZS*13S-1883 | 3.53 | 2.47 | 24.00 |
| 3891 | pZS*13S-1883 | 3.39 | 1.79 | 24.60 |
| 3891 | pZS*13S-1883 | 3.41 | 2.20 | 27.10 |
| 3891 | pZS*13S-1883 | 3.33 | 2.13 | 22.00 |
| 3891 | pZS*13S-1884 | 3.09 | 1.13 | 30.50 |
| 3891 | pZS*13S-1884 | 3.02 | 1.20 | 33.80 |
| 3891 | pZS*13S-1884 | 3.76 | 1.34 | 34.40 |
| 3891 | pZS*13S-1884 | 3.15 | 1.40 | 36.80 |
| 3891 | pZS*13S-1888 | 3.11 | 0.28 | 31.40 |
| 3891 | pZS*13S-1888 | 3.04 | 0.22 | 30.70 |
| 3891 | pZS*13S-1888 | 3.09 | 0.21 | 29.90 |
| 3891 | pZS*13S-1888 | 3.33 | 0.30 | 31.50 |
| 3891 | pZS*13S-1889 | 3.17 | 1.03 | 19.20 |
| 3891 | pZS*13S-1889 | 3.04 | 1.12 | 18.70 |
| 3891 | pZS*13S-1889 | 3.14 | 1.05 | 17.40 |
| 3891 | pZS*13S-1889 | 3.01 | 1.25 | 18.10 |
| 3891 | pZS*13S-1894 | 3.03 | 0.85 | 13.70 |
| 3891 | pZS*13S-1894 | 2.91 | 0.67 | 13.00 |
| 3891 | pZS*13S-1894 | 3.00 | 1.04 | 12.80 |
| 3891 | pZS*13S-1894 | 3.03 | 0.86 | 12.90 |

### Example XXVIII

### Production of 4111B and BDO Upon Expression of Genes Encoding 4-Hydroxybutyrate Transferase Enzymes

This example describes production of 4hb and BDO upon expression of genes encoding 4-hydroxybutyrate transferase enzymes.

Several heterologous 4-hydroxybutyrate transferase genes (Table 30) were expressed in E. coli from either the pZS*-13S or F′ plasmid to allow a functional pathway from alpha-ketoglutarate and succinyl-CoA to 1,4-butanediol. Host strains include 1269, 2731 Δcat2, and 1654. These host strains are based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). Strains 1269, 2731 Δcat2, and 1654 were all tested with 4-hydroxybutyryl-CoA reductase and 4-hydroxybutyraldehyde reductase expressed from the pZS*-13S plasmid. Tables 31, 32, and 33 show that expression of heterologous 4-hydroxybutyrate transferase genes allows BDO production in strains 1269, 2731 Δcat2, and 1654, respectively. Cells were cultured using the 96 well-plate protocol described previously.

TABLE 30
Genes encoding functional 4-hydroxybutyrate transferase enzymes.

| Gene | | | |
|---|---|---|---|
| Number | Organism | Accession | GI |
| 34 | Porphyromonas gingivalis W83 | NP_904965 | 34540486 |
| 1210 | Clostridium difficile 630 | YP_001088854 | 126699957 |
| 1769 | Eubacterium saphenum ATCC 49989 | ZP_05427217 | 255994082 |
| 1772 | Porphyromonas endodontalis | ZP_04389695 | 229495971 |
| | ATCC 35406 | | |
| 1774 | Anaerostipes caccae DSM 14662 | ZP_02417601 | 167745474 |
| 33B | Clostridium aminobutyricum | CAB60036 | 188032706 |
| 1801 | Fusobacterium nucleatum subsp. | ZP_00144049 | 34763077 |
| | vincentii ATCC 49256 | | |
| 1803 | Fusobacterium ulcerans ATCC 49185 | ZP_07928926 | 317064441 |
| 1804 | Fusobacterium varium ATCC 27725 | ZP_08693772 | 340757169 |
| 1805 | Odoribacter splanchnicus DSM 20712 | YP_004253244 | 325280702 |
| 1807 | Porphyromonas gingivalis | YP_001930004 | 188995752 |
| 1808 | Fusobacterium gonidiaformans | ZP_07914775 | 315918535 |
| | ATCC 25563 | | |
| 1809 | Acetonema longum DSM 6540 | ZP_08623452 | 338811223 |

The nucleotide sequences for 1210C and 033B are provided below as these were codon-optimized for expression in E. coli.

33B:
(SEQ ID NO: 174)
atggactggaagaagatctatgaagacagaacctgcactgcagatgaagcagtaaagagcattaagtcaggtgaccgcgtgctgtttgcgc
actgtgttgctgaaccgccagttctggtagaagcaatggttgcgaatgcagctgcatacaagaatgtcacggtttcacacatggttaccct
tggtaagggtgaatactcaaaaccagaatataaagaaaactttacttttgaaggttggtttaccagcccttcaacacgtggatccattgca
gaaggacacggacagtttgtccctgtattcttccacgaggtaccatctttaatccgtaaagacattttccatgttgatgtattcatggtaa
tggtatccccgccagatcataacggtttctgctgtgtgggtgtatcttctgactataccatgcaggctatcaaatcagcaaaaattgtact
ggctgaagtgaatgatcaggtacctgtagtttatggcgatacctttgttcacgttagtgaaatcgacaagttcgttgaaacttcacatcca
ctgccagaaatcggtctgccgaagatcggtgaagtagaagctgctattggtaagcactgcgcttcgctgatcgaagatggttccacattac
agcttggtatcggcgctattccggatgctgtactttcacagcttaaggacaagaaacaccttggtatccactctgaaatgatttccgacgg
tgttgttgatctttacgaagcaggcgttattgactgcagccaaaagtctatcgacaaaggcaaaatggcaataacattcttaatgggaacg
aagcgtctttatgatttcgctgcaaacaatccaaaggttgaattaaagccggttgactacatcaatcatccatctgtagttgcacagtgca
gcaaaatggtttgcatcaatgcttgcttgcaagttgattttatgggtcagattgtctccgatagtattggcacaaagcagttctccggcgt
aggcggtcaggttgacttcgtacgcggtgcatccatgtctattgacggcaaaggtaaagcgatcatcgcgatgccttccgttgcaaagaag
aaagatggcagtatgatttcgaagatcgttccattcatcgatcacggtgcagctgtaactacatcccgtaacgatgcggactatgtcgtaa
cggaatatggtattgctgaaatgaagggtaagtcgttacaggaccgcgcacgcgcgttaatcaatattgcccaccctgatttcaaagatga
attaaaggctgaatttgaaaagcgcttcaacgcggcattctaa.
1210C:
(SEQ ID NO: 175)
atgagctggcaagaactgtatcaaagtaaattatgttcagccacagaagcggtaaaacagattaaaaacggtgataccgtggtatttgccc
attgtgtaggtgaaccgcctgcactggtggaggcgatgattgaaaatgctgaacaatataaagatgttgagattaaacatatggttagcct
gggtagtggtggttatactgcgaaagggatggaagcgcattttcgcgtaaatccaatgtttgtcagcggcaatgtacgtaaggcgattgaa
aatggcgatggtgattttacacctgcattcttccatgaagtaccaaagttgctgcgtgaaaaacgtctgaaatgtgatgttgttctggcac
aggtaacgccaccagatgaacatggttattgttcgctgggaacaagcgttgattatacctatgaagccattaaaaccgcgcgcaccgtaat
tgttcaggtgaatgaccagtttcctcgcacctatggtgaggtggtgcatgtcagcgagtttgactatatcgttgaaaaatcacaaccgctg
tttgaactgcaacctgcaaagattggcgaagttgaagaagcgattggtaaaaattgtgcctcgctgattgaagatggtagcacgttacagc
tggggattggtgggattccggatgcggtgatgttatttctgactgataaaaaagatttagggattcatagcgaaatgattagcgatggcac
gctggcgctttatgaaaaaggtgttattaatggtaaatataaaaattttgataaagaaaaaatgacggttaccttcctgatgggtactaaa
aaactgtatgactttgccaataataacccggcagtagaggtaaaaccggtagactatgtgaatcatccggcaattatcatgaaacaacata
agatggtttctattaatagcgccattcaggttgatttaatggggcaggtggttgcagaggcgatgggactgcgccaattttccggtgttgg
cggtcaggttgactttattcgtggcgtgtcgatgggtgaagatggcaaggcgattatcgcgatgccttcaatcactacaaaaaaagatggt
acggtaattagcaaaatcgtctctattgtcgatgaaggtgcaccgattaccacctcacgtaatgatgttgattatattgtcacagaatacg
gtattgcagaattaaaaggcaaatcgctgcgtgaacgcgcacgtaatctgattaatattgctcatccatcggtacgtgaatcgctggcagt
agaatttgaaaagcgctttaaagagaaatattaa.

TABLE 31
BDO, GBL, and 4-hydroxybutyrate (4HB) production of E. coli host strain 1269 containing two
alternative 4-hydroxybutyrate transferase plasmid constructs. The plasmid construct denoted by only
“pZA33S (empty)” contains no 4-hydroxybutyrate transferase gene. Plasmid constructs denoted by F′
followed by a number express the 4-hydroxybutyrate transferase gene denoted by such number. BDO and
4HB concentrations are in mM.

| | | | LCMS-24 h | LCMS-24 h | LCMS-24 h |
|---|---|---|---|---|---|
| Host Strain | Plasmid | OD 24 h | 4HB | BDO | GBL |
| 1269 | pZA33S (empty) | 2.92 | 27.60 | 0.00 | 0.00 |
| 1269 | pZA33S (empty) | 2.12 | 23.80 | 0.59 | 0.07 |
| 1269 | pZA33S (empty) | 2.72 | 28.80 | 2.13 | 0.03 |
| 1269 | pZA33S (empty) | 2.32 | 30.50 | 0.14 | 0.00 |
| 1269 | F′34 | 1.76 | 2.42 | 22.20 | 0.87 |
| 1269 | F′34 | 1.70 | 2.34 | 21.00 | 0.88 |
| 1269 | F′34 | 2.09 | 3.23 | 23.50 | 1.21 |
| 1269 | F′34 | 3.83 | 2.77 | 51.30 | 5.01 |
| 1269 | F′1210C | 1.99 | 2.84 | 28.20 | 1.73 |
| 1269 | F′1210C | 1.64 | 2.73 | 27.30 | 1.61 |
| 1269 | F′1210C | 1.89 | 4.28 | 32.30 | 2.59 |
| 1269 | F′1210C | 3.47 | 3.76 | 56.38 | 7.22 |

TABLE 32
BDO, GBL, and 4-hydroxybutyrate (4HB) production of E. coli host strain 2731 Δcat2 containing
alternative 4-hydroxybutyrate transferase plasmid constructs. Plasmid constructs denoted by pZS*13S-
followed by a number express the 4-hydroxybutyrate transferase gene denoted by such number. The
pZS*13S-backbone also expresses functional 4-hydroxybutyryl-CoA reductase and 4-hydroxybutyraldehyde
dehydrogenase genes. BDO and 4HB concentrations are in mM.

| | | | LCMS-19 h | LCMS-19 h | LCMS-19 h |
|---|---|---|---|---|---|
| Host Strain | Plasmid | OD 19 h | 4HB | BDO | GBL |
| 2731 Δcat2 | pZS*13S-1769 | 3.75 | 1.1 | 24.9 | 0.1 |
| 2731 Δcat2 | pZS*13S-1769 | 3.84 | 1.3 | 23.9 | 0.1 |
| 2731 Δcat2 | pZS*13S-1769 | 4.10 | 1.3 | 23.0 | 0.1 |
| 2731 Δcat2 | pZS*13S-1769 | 4.14 | 1.1 | 27.3 | 0.1 |
| 2731 Δcat2 | pZS*13S-1772 | 4.33 | 2.9 | 36.7 | 0.2 |
| 2731 Δcat2 | pZS*13S-1772 | 4.37 | 2.3 | 37.9 | 0.3 |
| 2731 Δcat2 | pZS*13S-1772 | 4.02 | 2.7 | 40.4 | 0.3 |
| 2731 Δcat2 | pZS*13S-1772 | 3.59 | 3.4 | 27.4 | 0.2 |
| 2731 Δcat2 | pZS*13S-1774 | 4.10 | 2.2 | 29.9 | 0.2 |
| 2731 Δcat2 | pZS*13S-1774 | 3.93 | 1.9 | 29.2 | 0.3 |
| 2731 Δcat2 | pZS*13S-1774 | 4.26 | 2.5 | 31.5 | 0.3 |
| 2731 Δcat2 | pZS*13S-1774 | 4.76 | 1.6 | 29.8 | 0.2 |

TABLE 33
BDO, GBL, and 4-hydroxybutyrate (4HB) production of E. coli host strain 1654 containing
alternative 4-hydroxybutyrate transferase plasmid constructs. Plasmid constructs denoted by pZS*13S-
followed by a number express the 4-hydroxybutyrate transferase gene denoted by such number. The
pZS*13S-backbone also expresses functional 4-hydroxybutyryl-CoA reductase and 4-hydroxybutyraldehyde
dehydrogenase genes. BDO and 4HB concentrations are in mM.

| | | | LCMS-19 h | LCMS-19 h | LCMS-19 h |
|---|---|---|---|---|---|
| Host Strain | Plasmid | OD 19 h | 4HB | BDO | GBL |
| 1654 | pZS*-13S-033B | 3.96 | 1.47 | 26.00 | 1.69 |
| 1654 | pZS*-13S-033B | 4.17 | 1.32 | 23.90 | 1.54 |
| 1654 | pZS*-13S-033B | 4.11 | 0.99 | 25.80 | 1.62 |
| 1654 | pZS*-13S-033B | 3.80 | 1.04 | 25.40 | 1.61 |
| 1654 | pZS*-13S-1801 | 3.68 | 1.88 | 21.80 | 1.61 |
| 1654 | pZS*-13S-1801 | 3.90 | 1.35 | 21.70 | 1.67 |
| 1654 | pZS*-13S-1801 | 3.76 | 1.76 | 20.90 | 1.55 |
| 1654 | pZS*-13S-1801 | 3.66 | 1.92 | 22.00 | 1.77 |
| 1654 | pZS*-13S-1803 | 3.69 | 1.71 | 27.60 | 1.80 |
| 1654 | pZS*-13S-1803 | 3.79 | 1.45 | 23.60 | 1.63 |
| 1654 | pZS*-13S-1803 | 3.64 | 1.09 | 24.80 | 1.72 |
| 1654 | pZS*-13S-1803 | 3.64 | 1.30 | 23.60 | 1.67 |
| 1654 | pZS*-13S-1804 | 3.74 | 1.27 | 26.00 | 1.83 |
| 1654 | pZS*-13S-1804 | 3.87 | 1.90 | 25.40 | 1.95 |
| 1654 | pZS*-13S-1804 | 4.04 | 1.48 | 22.80 | 1.65 |
| 1654 | pZS*-13S-1804 | 3.75 | 1.87 | 26.00 | 1.87 |
| 1654 | pZS*-13S-1805 | 3.84 | 1.47 | 25.20 | 1.47 |
| 1654 | pZS*-13S-1805 | 3.75 | 1.06 | 27.50 | 1.54 |
| 1654 | pZS*-13S-1805 | 3.08 | 1.67 | 22.00 | 1.26 |
| 1654 | pZS*-13S-1805 | 3.57 | 1.22 | 23.80 | 1.36 |
| 1654 | pZS*-13S-1807 | 3.24 | 0.00 | 4.80 | 0.40 |
| 1654 | pZS*-13S-1807 | 3.30 | 0.29 | 5.10 | 0.48 |
| 1654 | pZS*-13S-1807 | 3.36 | 0.17 | 4.68 | 0.39 |
| 1654 | pZS*-13S-1807 | 3.31 | 0.21 | 5.16 | 0.38 |
| 1654 | pZS*-13S-1808 | 3.38 | 2.39 | 24.50 | 1.20 |
| 1654 | pZS*-13S-1808 | 3.39 | 2.56 | 22.20 | 1.07 |
| 1654 | pZS*-13S-1808 | 3.45 | 2.16 | 22.40 | 1.10 |
| 1654 | pZS*-13S-1808 | 3.42 | 2.26 | 23.10 | 1.27 |
| 1654 | pZS*-13S-1809 | 4.26 | 1.52 | 18.10 | 0.93 |
| 1654 | pZS*-13S-1809 | 4.37 | 1.86 | 21.50 | 1.07 |
| 1654 | pZS*-13S-1809 | 3.51 | 1.67 | 16.00 | 0.81 |
| 1654 | pZS*-13S-1809 | 4.49 | 0.97 | 18.60 | 0.97 |

### EXAMPLE XXIX

### Production of 41113 and BDO Upon Expression of Genes Encoding 4-Hydroxybutyryl-CoA Reductase Enzymes

This example describes production of 4hb and BDO upon expression of genes encoding 4-hydroxybutyryl-CoA reductase enzymes.

Several heterologous 4-hydroxybutyryl-CoA reductase genes (Table 34) were expressed in E. coli from the pZS*-13S plasmid to allow afunctional pathway from succinyl-CoA and alpha-ketoglutarate to 1,4-butanediol. Host strain 1889 is based on strain ECKh432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis(encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). Strain 1889 contains deletions in the native alcohol dehydrogenase genes adhP, yqhD, yahK, and yjgB. Additionally, strain 1889 contains a chromosomally integrated copy of 4-hydroxybutyryl-CoA transferase expressed from the pA promoter at the attB locus. Strain 1889 contains the F′ plasmid expressing functional 4-hydroxybutyraldehyde reductase gene. Strain 4269 contains a chromosomally integrated copy of 4-hydroxybutyraldehyde reductase gene at the hemN locus under control of the p119 promoter. Tables 35 and 36 show that expression of heterologous 4-hydroxybutyryl-CoA reductase genes enhances BDO production in strains 1889 and 4269, respectively, over the controls denoted by “pZS*13 S”. Cells were cultured using the 96 well-plate protocol described previously.

TABLE 34
Genes encoding functional 4-hydroxybutyryl-CoA reductase enzymes.

| Gene | | | |
|---|---|---|---|
| Number | Organism | Accession | GI |
| 025B | Clostridium beijerinckii NCIMB | YP_001310903.1 | 150018649 |
| | 8052 | | |
| 733 | Clostridium hylemonae DSM 15053 | ZP_03778292.1 | 225569267 |
| 744 | Clostridium methylpentosum DSM | ZP_03705305.1 | 225016072 |
| | 5476 | | |
| 787 | Eubacterium hallii DSM 3353 | ZP_03715465.1 | 225026273 |
| 865 | Ruminococcus obeum ATCC 29174 | ZP_01962381.1 | 153809713 |
| 778 | Bacillus selenitireducens MLS10 | YP_003701164.1 | 297585384 |
| 714 | Clostridium | AAP42563.1 | 31075383 |
| | saccharoperbutylacetonicum | | |
| | N1-4 | | |
| 707 | Lactobacillus brevis ATCC 367 | YP_795711.1 | 116334184 |
| 141 | Desulfatibacillum alkenivorans | YP_002434126.1 | 218782808 |
| | AK-01 | | |
| 133 | Clostridium phytofermentans ISDg | YP_001558295.1 | 160879327 |
| 715 | Clostridium bolteae ATCC | ZP_02089671.1 | 160942363 |
| | BAA-613 | | |
| 779 | Photobacterium profundum 3TCK | ZP_01222600.1 | 90414628 |
| 145 | Citrobacter koseri ATCC BAA-895 | YP_001452373.1 | 157145054 |
| 704 | Salmonella enterica typhimurium | NP_460996.1 | 16765381 |
| 777 | Sebaldella termitidis ATCC 33386 | YP_003307836.1 | 269119659 |
| 795 | Fusobacterium nucleatum subsp. | ZP_04969437.1 | 254302079 |
| | polymorphum ATCC 10953 | | |
| 706 | Tolumonas auensis DSM 9187 | YP_002892893.1 | 237808453 |
| 137 | Rhodospirillum rubrum ATCC | YP_426002.1 | 83592250 |
| | 11170 | | |
| 1993 | Metalibrary sp | N/A | N/A |

TABLE 35
BDO and 4-hydroxybutyrate (4HB) production of E. coli host strain 1889 containing several
alternative 4-hydroxybutyryl-CoA reductase plasmid constructs. The plasmid construct denoted by only
“pZS*13S” contains no 4-hydroxybutyryl-CoA reductase gene. Plasmid constructs denoted by pZS*13S
followed by a number express the 4-hydroxybutyryl-CoA reductase gene denoted by such number. BDO and
4HB concentrations are in mM.

| | | | LCMS-24 hrs | LCMS-24 hrs |
|---|---|---|---|---|
| Host | Plasmid | OD 24 h | 4HB | BDO |
| 1889 | pZS*13S | 3.12 | 3.93 | 0 |
| 1889 | pZS*13S | 3.68 | 10.4 | 0.5 |
| 1889 | pZS*13S | 2.81 | 2.74 | 0 |
| 1889 | pZS*13S | 2.61 | 1.16 | 0 |
| 1889 | pZS*13S-025B | 3.25 | 0.87 | 8.42 |
| 1889 | pZS*13S-025B | 2.11 | 0.8 | 7.09 |
| 1889 | pZS*13S-025B | 3.01 | 0.65 | 7.38 |
| 1889 | pZS*13S-025B | 3.04 | 0.72 | 6.81 |
| 1889 | pZS*13S-733 | 1.67 | 1.07 | 1.64 |
| 1889 | pZS*13S-733 | 1.63 | 1.22 | 1.87 |
| 1889 | pZS*13S-733 | 2.16 | 1.16 | 1.89 |
| 1889 | pZS*13S-733 | 2.34 | 1.04 | 1.88 |
| 1889 | pZS*13S-744 | 2.75 | 0.63 | 2.17 |
| 1889 | pZS*13S-744 | 3.00 | 0.65 | 1.75 |
| 1889 | pZS*13S-744 | 2.75 | 0.5 | 1.72 |
| 1889 | pZS*13S-744 | 1.98 | 0.39 | 0.56 |
| 1889 | pZS*13S-787 | 2.63 | 0.88 | 1.82 |
| 1889 | pZS*13S-787 | 2.40 | 1 | 1.75 |
| 1889 | pZS*13S-787 | 2.88 | 1.06 | 2.41 |
| 1889 | pZS*13S-787 | 3.26 | 1.03 | 1.86 |
| 1889 | pZS*13S-865 | 2.60 | 0.95 | 1.71 |
| 1889 | pZS*13S-865 | 2.70 | 1.11 | 2.09 |
| 1889 | pZS*13S-865 | 2.69 | 0.98 | 1.4 |
| 1889 | pZS*13S-865 | 2.70 | 1.06 | 1.74 |
| 1889 | pZS*13S-778 | 2.76 | 1.15 | 5.79 |
| 1889 | pZS*13S-778 | 3.12 | 1.06 | 6.48 |
| 1889 | pZS*13S-778 | 2.83 | 1.08 | 4.98 |
| 1889 | pZS*13S-778 | 2.79 | 1.1 | 5.19 |
| 1889 | pZS*13S-714 | 2.60 | 0.63 | 6.55 |
| 1889 | pZS*13S-714 | 2.77 | 0.86 | 6.88 |
| 1889 | pZS*13S-714 | 2.67 | 0.77 | 6.51 |
| 1889 | pZS*13S-714 | 2.73 | 0.75 | 8.12 |
| 1889 | pZS*13S-707 | 1.39 | 0.3 | 1.56 |
| 1889 | pZS*13S-707 | 1.05 | 0.24 | 1.57 |
| 1889 | pZS*13S-707 | 1.99 | 0.26 | 1.62 |
| 1889 | pZS*13S-707 | 2.20 | 0.28 | 1.76 |
| 1889 | pZS*13S-141 | 2.54 | 1.08 | 2.13 |
| 1889 | pZS*13S-141 | 2.72 | 1 | 2.01 |
| 1889 | pZS*13S-141 | 2.54 | 1.04 | 1.9 |
| 1889 | pZS*13S-141 | 2.34 | 0.97 | 1.77 |
| 1889 | pZS*13S-133 | 2.63 | 0.7 | 2.69 |
| 1889 | pZS*13S-133 | 1.79 | 0.73 | 2.5 |
| 1889 | pZS*13S-133 | 3.04 | 0.69 | 2.26 |
| 1889 | pZS*13S-133 | 3.21 | 0.65 | 2.84 |
| 1889 | pZS*13S-715 | 2.79 | 1.03 | 2.31 |
| 1889 | pZS*13S-715 | 2.67 | 1.13 | 1.55 |
| 1889 | pZS*13S-715 | 2.90 | 1.02 | 1.6 |
| 1889 | pZS*13S-715 | 3.10 | 1.17 | 2.26 |
| 1889 | pZS*13S-779 | 3.21 | 8.88 | 0.46 |
| 1889 | pZS*13S-779 | 2.84 | 10.5 | 0.63 |
| 1889 | pZS*13S-779 | 3.98 | 11.9 | 0.84 |
| 1889 | pZS*13S-779 | 4.55 | 13.5 | 1.15 |
| 1889 | pZS*13S-145 | 2.08 | 0.53 | 0.76 |
| 1889 | pZS*13S-145 | 2.65 | 0.66 | 0.72 |
| 1889 | pZS*13S-145 | 2.74 | 0.55 | 0.63 |
| 1889 | pZS*13S-145 | 3.08 | 0.7 | 0.68 |
| 1889 | pZS*13S-704 | 2.49 | 0.93 | 1.65 |
| 1889 | pZS*13S-704 | 2.08 | 0.89 | 1.81 |
| 1889 | pZS*13S-704 | 2.97 | 0.76 | 1.58 |
| 1889 | pZS*13S-704 | 3.35 | 0.9 | 2.32 |
| 1889 | pZS*13S-777 | 2.65 | 1.45 | 0.94 |
| 1889 | pZS*13S-777 | 3.03 | 1.89 | 1.5 |
| 1889 | pZS*13S-777 | 3.36 | 2.16 | 1.79 |
| 1889 | pZS*13S-777 | 3.10 | 1.93 | 1.6 |
| 1889 | pZS*13S-795 | 2.60 | 3.22 | 1.55 |
| 1889 | pZS*13S-795 | 3.43 | 4.54 | 1.96 |
| 1889 | pZS*13S-795 | 3.33 | 4.24 | 1.93 |
| 1889 | pZS*13S-795 | 2.87 | 2.15 | 0.97 |
| 1889 | pZS*13S-706 | 2.67 | 0.96 | 1.36 |
| 1889 | pZS*13S-706 | 2.41 | 1.52 | 2.64 |
| 1889 | pZS*13S-706 | 2.94 | 1.4 | 2.56 |
| 1889 | pZS*13S-706 | 2.71 | 0.85 | 0.89 |
| 1889 | pZS*13S-137 | 2.85 | 1.76 | 1.4 |
| 1889 | pZS*13S-137 | 3.04 | 2.31 | 2.21 |
| 1889 | pZS*13S-137 | 3.00 | 1.89 | 1.56 |
| 1889 | pZS*13S-137 | 2.45 | 0.55 | 0.17 |

TABLE 36
BDO and 4-hydroxybutyrate (4HB) production of E. coli host strain 4269 containing
either no heterologous 4-hydroxybutyryl-CoA reductase gene (i.e., pZS*13S) or
expressing the 4-hydroxybutyryl-CoA reductase gene, 1993, from the pZS* plasmid
under control of the p100 promoter. The gene sequence for 1993 is provided in the
bottom column of the table. BDO and 4HB concentrations are in mM.

| | | | LCMS-24 hrs | LCMS-24 hrs |
|---|---|---|---|---|
| Host | Plasmid | OD 24 h | 4HB | BDO |
| 4269 | pZS*13S | 3.12 | 49.3 | 1.9 |
| 4269 | pZS*13S | 3.08 | 50.7 | 1.7 |
| 4269 | pZS*13S | 3.31 | 48.2 | 3.0 |
| 4269 | pZS*13S | 3.18 | 55.7 | 3.2 |
| 4269 | pZS*13S-p100-1993 | 3.88 | 13.1 | 37.2 |
| 4269 | pZS*13S-p100-1993 | 3.58 | 18.3 | 44.3 |
| 4269 | pZS*13S-p100-1993 | 3.40 | 17.3 | 46.9 |
| 4269 | pZS*13S-p100-1993 | 3.40 | 16.0 | 46.5 |

Gene sequence for 1993:
atggatttgaagctgaccgatgcggacgttgaggcaatcgtagcgcaagtcatggctaacattgagcgccgtctgggtagcgcggaagcg
ggcagcgcagcatccgctgcgtcgccggcaccggctgcgccggttcgtaccgctccgggtggcagcccggcagccagcccgcgtccg
gagtacggtgtttttgatcgtgcggaggatgcagtcgccgctgccgccgaagctcaggaagccttcctgcgccagtgtcgtctgcaagacc
gtgagcgcattctgcgtgccatccgtgaagagactctggcacgcaaggaagaattggcacgcctgatttgggaggaaacgaagctgggtc
gcttggaacacaaaattgcaaagctggaattgacggcgctgaaaaccccgggtacggaggatctgcgcaccgaggcatttagcggcgac
aacggcctgaccatcgtcgaacatgcgccgtacggtgtgattggcgcggttaccccggttacgaaccctgcggagactattatcaacaacg
cgatcggcatgctggcaagcggtaatgcagtggtgttcaacgtgcaccctagcgctaaacgttgctccgcgtataccgtccagatgatcaat
aaagcagtgatggcagcgggtggtccgccgaatctggttacgatggttcgcgagccgaccatggaaaccctgaatgcgatcatccgcagc
ccgagcgtgaagctgctggtgggcaccggcggtccgggtctggttagcaccctgctgcgctctggtaagaaagcgattggtgcgggtgc
gggcaatccgccggtcgttgtggatgataccgccgacctggagcatgcggcaaaggaaatcatcaaaggcgcgtcctttgacaacaatatt
ctgtgtattgcggagaaagaggtttttgtggttgataaagcggccgacggtctgatctatcacatgctggataacggcgcatacatgctgggt
cgtgacgagctggagaaggtgatgcaattcgcgctgaccgcggacgaaagccagggcggtgcgggttgctctattgatccgcgtcgtgc
gtggcatgtgactaaggagtggggggcaaagatgcgcgcttgttcctggagaagattggtgtcaagactgaccgtccggttgacttgctgt
tgtgcgaggttgactttgaccacccgttcgtgcagctggaacaaatgatgccagtactgccgattgttcgtgtccgtgacctggatgaagcca
tcggtatggccgtccgtgcggagcacggcaatcgtcacaccgcaattatgcatagccgtaacgtggacaatctgacccgttttgcccgtgcc
attgccacgaccatcttcgtcaaaaacgcaagcagcttggcgggtgttggttatggcggtgaaggttttaccaccatgacgategccggtcc
aacgggcgagggtctgacgtcggctcgtacgttcacgcgcaaagtccgctgtgtcctggcggacggcggtttccgtatcgttggttaa
(SEQ ID NO: 176)

### Example XXX

### Production of 4HB and BDO Upon Expression of Genes Encoding 4-Hydroxybutyraldehyde Reductase Enzymes

This example describes production of 4hb and BDO upon expression of genes encoding 4-hydroxybutyraldehyde reductase enzymes.

Several heterologous 4-hydroxybutyraldehyde reductase genes (Table 37) were expressed in E. coli from the pZS*-13S plasmid to allow a functional pathway from succinyl-CoA and alpha-ketoglutarate to 1,4-butanediol. Host strain 1889 is based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). Strain 1889 contains deletions in the native alcohol dehydrogenase genes adhP, yqhD, yahK, and yjgB. Additionally, strain 1889 contains a chromosomally integrated copy of 4-hydroxybutyryl-CoA transferase expressed from the pA promoter at the attB locus. Table 38 shows that expression of heterologous 4-hydroxybutyraldehyde reductase genes enhances BDO production in strain 1889 over the controls denoted by “pZS*13S”. Cells were cultured using the 96 well-plate protocol described previously.

TABLE 37
Genes encoding functional 4-hydroxybutyraldehyde reductase enzymes.

| Gene Number | Organism | Accession | GI |
|---|---|---|---|
| 12 | Escherichia coli | AAC76047.1 | 1789386 |
| | K-12 MG1655 | | |
| 956 | Clostridium beijerinckii | YP_001309304.1 | 150017050 |
| | NCIMB 8052 | | |
| 1247 | Clostridium | P13604.1 | 113352 |
| | saccharobutylicum | | |
| 1248 | Clostridium asparagiforme | ZP_03760651.1 | 225405462 |
| | DSM 15981 | | |
| 1250 | Clostridium bolteae | ZP_02083621.1 | 160936248 |
| | ATCC BAA-613 | | |
| 1251 | Clostridium | YP_003845251.1 | 302876618 |
| | cellulovorans 743B | | |
| 1256 | Clostridium hiranonis | ZP_03294286.1 | 210624270 |
| | DSM 13275 | | |
| 1259 | Clostridium | ZP_03705769.1 | 225016577 |
| | methylpentosum | | |
| | DSM 5476 | | |
| 1364 | Atopobium parvulum | YP_003179160.1 | 257783943 |
| | DSM 20469 | | |
| 1365 | Tolumonas auensis | YP_002893476.1 | 237809036 |
| | DSM 9187 | | |
| 1366 | Clostridium | ZP_05394983.1 | 255528157 |
| | carboxidivorans P7 | | |

TABLE 38
BDO and 4-hydroxybutyrate (4HB) production of E. coli host
strain 1889 containing several alternative 4-hydroxybutyraldehyde
reductase plasmid constructs. The plasmid construct
denoted by only “pZS*13S-” contains no 4-hydroxybutyraldehyde
reductase gene but does express a 4-hydroxybutyryl-CoA
reductase gene. Plasmid constructs denoted by pZS*13S-
followed by a number express both 4-hydroxybutyryl-CoA
reductase and the 4-hydroxybutyraldehyde reductase
gene denoted by such number. BDO and 4HB concentrations
are in mM.

| | | | LCMS-24 h | LCMS-24 h |
|---|---|---|---|---|
| Host | Plasmid | OD 24 h | 4HB | BDO |
| 1889 | pZS*13S | 4.68 | 21.5 | 0.4 |
| 1889 | pZS*13S | 4.33 | 15.9 | 0.1 |
| 1889 | pZS*13S | 4.83 | 18.9 | 0.3 |
| 1889 | pZS*13S | 4.25 | 19.8 | 0.6 |
| 1889 | pZS*13S-956 | 4.45 | 2.4 | 52.0 |
| 1889 | pZS*13S-956 | 4.92 | 3.5 | 52.1 |
| 1889 | pZS*13S-956 | 4.65 | 4.2 | 42.9 |
| 1889 | pZS*13S-956 | 5.37 | 4.7 | 54.2 |
| 1889 | pZS*13S-1247 | 4.99 | 2.4 | 46.6 |
| 1889 | pZS*13S-1247 | 5.38 | 2.3 | 42.5 |
| 1889 | pZS*13S-1247 | 5.68 | 2.1 | 38.6 |
| 1889 | pZS*13S-1247 | 5.18 | 2.0 | 40.4 |
| 1889 | pZS*13S-1248 | 4.25 | 2.8 | 17.6 |
| 1889 | pZS*13S-1248 | 4.4 | 1.9 | 17.8 |
| 1889 | pZS*13S-1248 | 3.06 | 2.1 | 17.3 |
| 1889 | pZS*13S-1248 | 4.22 | 2.0 | 19.5 |
| 1889 | pZS*13S-1250 | 4.76 | 0.9 | 30.4 |
| 1889 | pZS*13S-1250 | 5.05 | 1.0 | 30.9 |
| 1889 | pZS*13S-1250 | 4.67 | 1.1 | 28.5 |
| 1889 | pZS*13S-1250 | 5.57 | 1.1 | 33.5 |
| 1889 | pZS*13S-1251 | 4.68 | 2.0 | 49.9 |
| 1889 | pZS*13S-1251 | 4.65 | 2.0 | 45.3 |
| 1889 | pZS*13S-1251 | 4.86 | 1.7 | 42.6 |
| 1889 | pZS*13S-1251 | 4.76 | 1.9 | 42.7 |
| 1889 | pZS*13S-1256 | 4.63 | 15.7 | 11.9 |
| 1889 | pZS*13S-1256 | 4.48 | 16.2 | 12.6 |
| 1889 | pZS*13S-1256 | 5.1 | 15.0 | 12.8 |
| 1889 | pZS*13S-1256 | 5.51 | 15.3 | 13.4 |
| 1889 | pZS*13S-1259 | 5.01 | 1.0 | 33.7 |
| 1889 | pZS*13S-1259 | 5.29 | 1.1 | 28.9 |
| 1889 | pZS*13S-1259 | 5.49 | 1.3 | 29.5 |
| 1889 | pZS*13S-1259 | 5.25 | 1.2 | 31.5 |
| 1889 | pZS*13S-1364 | 5.52 | 5.0 | 48.8 |
| 1889 | pZS*13S-1364 | 5.12 | 8.3 | 47.7 |
| 1889 | pZS*13S-1364 | 5.47 | 7.1 | 42.2 |
| 1889 | pZS*13S-1364 | 5.48 | 7.8 | 46.0 |
| 1889 | pZS*13S-1365 | 5.49 | 1.1 | 33.1 |
| 1889 | pZS*13S-1365 | 5.65 | 0.7 | 37.7 |
| 1889 | pZS*13S-1365 | 6.08 | 1.1 | 34.8 |
| 1889 | pZS*13S-1365 | 5.5 | 0.9 | 28.6 |
| 1889 | pZS*13S-1366 | 5.13 | 2.2 | 52.6 |
| 1889 | pZS*13S-1366 | 4.76 | 2.4 | 56.1 |
| 1889 | pZS*13S-1366 | 4.93 | 2.2 | 56.9 |
| 1889 | pZS*13S-1366 | blank | 2.5 | 57.9 |

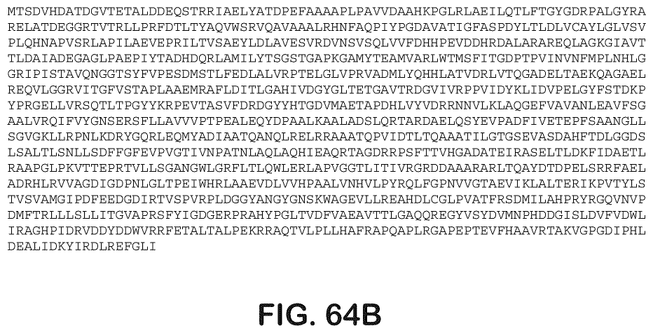

In the examples below, additional genetic manipulations are described that were made in the host strain to improve the yield of BDO on glucose. The key genes for deletion and overexpression are shown in FIG. 70.

Starting from the TCA cycle intermediate, alpha-ketoglutarate (AKG), there are two ways to channel flux into the BDO pathway. The first one comprises of an α-ketoacid decarboxylase that decarboxylates AKG into succinate semialdehyde and subsequently reduces it to 4-hydroxybutyrate (4HB). An alternative way to get to 4HB is via AKG dehydrogenase which transforms AKG into succinyl-CoA. This is then reduced to succinate semialdehyde via succinate semialdehyde dehydrogenase and then to 4HB. 4HB can then be activated to 4-hydroxybutyryl-CoA (4-HBCoA) via a transferase that transfers the CoA from acetyl-CoA. 4-HBCoA is reduced to 4-hydroxybutyraldehyde via an aldehyde dehydrogenase (ALD). The aldehyde is finally reduced to 1,4-butanediol (BDO) using an alcohol dehydrogenase (ADH).

### Example XXXI

### Overexpression of Phosphoenolpyruvate Carboxylase

This example describes overexpression of phosphoenolpyruvate (PEP) carboxylase. See corresponding example in WO2013/186402.

ppc refers to a gene that encodes for phosphoenolpyruvate (PEP) carboxylase activity. The net reaction involves the conversion of PEP and bicarbonate into oxaloacetate and phosphate. The overexpression of PEP carboxylase leads to conversion of more phosphoenolpyruvate (PEP) into OAA, thus reducing the flux from PEP into pyruvate and subsequently into acetyl-CoA. This leads to increased flux into the TCA cycle and thus into the pathway. Further, this overexpression also decreases the intracellular acetyl-CoA pools available for the ethanol-forming enzymes to work with, thus reducing the formation of ethanol and acetate. The increased flux towards oxaloacetate also helps reduce pyruvate and alanine secretion. Additionally, increased availability of oxaloacetate can reduce the cellular needs for respiration and also the amount of carbon lost as CO₂. The gene ppc was cloned on the plasmid pZS*-13S as described above.

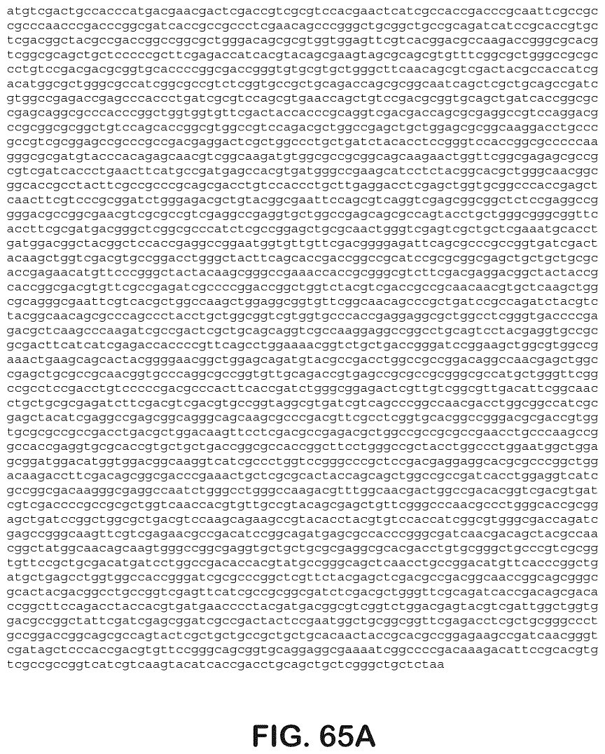

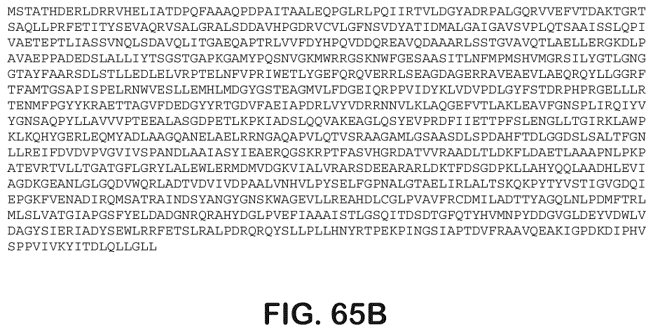

Table 39 shows the BDO production in 96-well plates from the strain 3162 from 4 replicates. The gene ppc (#525) was overexpressed on pZS* with a pA promoter as described before and an RBK variant 5K was created. Host strain 3162 is based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). Strain 3162 contains deletions in the native alcohol dehydrogenase genes adhP, yqhD, yahK, and yjgB. Additionally, strain 3162 contains a chromosomally integrated copy of 4-hydroxybutyryl-CoA transferase expressed from the pA promoter at the attB locus. It also has deletions of cytochrome oxidases, cyoABCD and appBC. The aldehyde dehydrogenase and alcohol dehydrogenase genes for converting 4-hydroxybutyryl-CoA into 4-hydroxybutyraldehyde and subsequently to 1,4-butanediol (BDO) are expressed on the pPSX23R plasmid. The gene candidates for these enzyme steps have already been described. FIG. 71 shows the average BDO and 4HB for the culture and compares it to 4 replicates from the control strain 3162. The same comparison is made in FIG. 72 for the ethanol numbers. Higher BDO and 4HB were observed in cells with ppc overexpressed. As expected, lower ethanol numbers were seen.

TABLE 39
BDO, 4-hydroxybutyrate (4HB) and ethanol data for four 4HB-producing
replicates that had the gene ppc overexpressed on pZS* after
24 hours of culture time in 96-well plates. The top 4 rows show the
replicate data for the control cultures. All the concentrations are in mM.

| Host | Plasmid #1 | OD | 4HB | BDO | EtOH |
|---|---|---|---|---|---|
| 3162 | pZS*135 | 3.08 | 5.16 | 23.3 | 2.11 |
| 3162 | pZS*135 | 2.96 | 5.23 | 24.5 | 2.27 |
| 3162 | pZS*135 | 2.76 | 4.98 | 25.2 | 2.23 |
| 3162 | pZS*135 | 3.05 | 6.02 | 26 | 2.3 |
| 3162 | pZS*135-(5k)525 | 3.02 | 9.18 | 29.9 | 1.59 |
| 3162 | pZS*135-(5k)525 | 2.50 | 9.32 | 28.8 | 1.47 |
| 3162 | pZS*135-(5k)525 | 3.24 | 9.3 | 28 | 1.49 |
| 3162 | pZS*135-(5k)525 | 3.38 | 24.5 | 0.72 | 0.69 |

### Example XXXII

### Overexpression of Alpha-ketoglutarate Dehydrogenase

This example describes overexpression of alpha-ketoglutarate dehydrogenase. See corresponding example in WO2013/186402.

This enzyme complex is formed by sucA, sucB and lpdA in Escherichia coli. It converts alpha-ketoglutarate into succinyl-CoA that is subsequently channeled into the BDO pathway. A limitation in this pathway, including any limitation in the capacity of alpha-ketoglutarate dehydrogenase, can lead to the formation of glutamate via, for example, glutamate dehydrogenase (gdhA). This leads to a carbon loss and reduction in yield. Expressing an extra copy of sucAB and lpdA in the BDO-producing strain helped reduce glutamate by significant levels. It can also pull flux through the TCA cycle and help reduce other C2 (ethanol and acetate) and C3 byproducts (alanine, pyruvate). sucAB and lpdA were cloned on pZS* plasmid under the pA promoter as described above. ALD and ADH required for BDO production were expressed on pPSX23R as described earlier.

Table 40 shows the 4HB and BDO production in 96-well plates from 4 replicates of strain 3933 after 24 hours of culture time. Host strain 3933 is based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). It contains deletions in the native alcohol dehydrogenase genes adhP, yqhD, yahK, and yjgB. The strain contains a chromosomally integrated copy of 4-hydroxybutyryl-CoA transferase expressed from the pA promoter at the attB locus. It also has deletions of cytochrome oxidases, cyoABCD and appBC, and adh is integrated on the chromosome under the promoter p119 at the hemN locus.

TABLE 40
BDO, 4HB and ethanol data for four 4HB producing
replicates of strain 3933 that had the genes
sucAB-lpdA overexpressed on a plasmid. The bottom 4 rows
show the replicate data for the control cultures of
the host strain 3933. All the concentrations are in mM and
were measured after 24 hours of culture time in 96-
well plates. 1438 refers to the native sucA gene
(b0726), 1439 refers to the native wild type sucB gene
(b0727), the last gene in the biobrick on pZS* is mutant
version of lpdA (b0116) described previously in
Example XIV This gene is a subunit of the pyruvate
dehydrogenase complex in Escherichia coli and has been
modified such that pyruvate dehydrogenase is not inhibited
by NADH. The mutations were made as described
in Vim et al., Nature Chemical Biology 7:445-452 (2011)).

| Host | plasmid_1 | OD | 4HB | BDO | Glutamate |
|---|---|---|---|---|---|
| 3933 | pZS*13S_1438-1439- | 2.91 | 6.27 | 35.00 | 0.03 |
| | 160D345K-lpdA-KP | | | | |
| 3933 | pZS*13S_1438-1439- | 2.89 | 6.37 | 33.50 | 0.03 |
| | 160D345K-lpdA-KP | | | | |
| 3933 | pZS*13S_1438-1439- | 2.94 | 6.52 | 32.40 | 0.04 |
| | 160D345K-lpdA-KP | | | | |
| 3933 | pZS*13S_1438-1439- | 2.96 | 5.76 | 31.10 | 0.04 |
| | 160D345K-lpdA-KP | | | | |
| 3933 | pZS*13S | 3.06 | 6.44 | 38.30 | 2.68 |
| 3933 | pZS*13S | 3.05 | 5.42 | 36.40 | 2.20 |
| 3933 | pZS*13S | 3.22 | 5.36 | 36.40 | 2.21 |
| 3933 | pZS*13S | 3.12 | 4.95 | 35.00 | 2.08 |

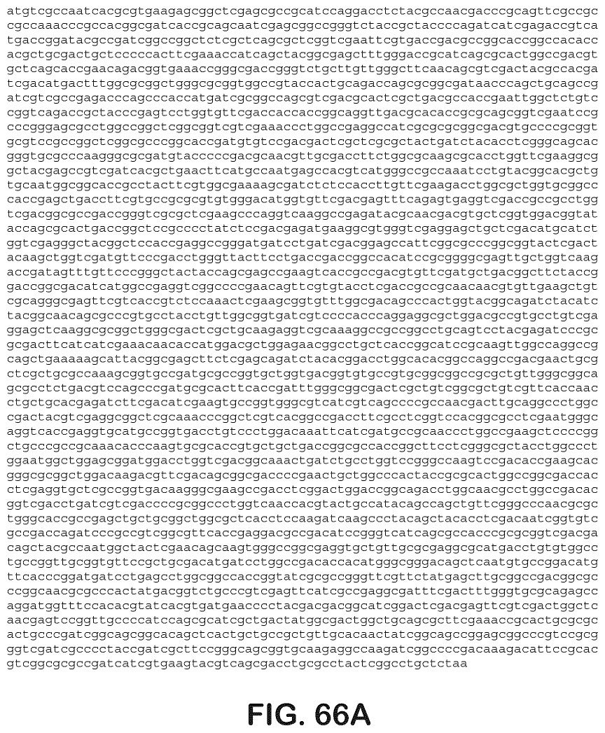

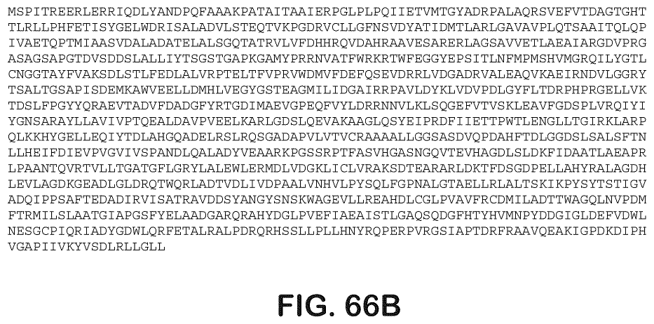

FIG. 73 shows the average BDO and 4HB numbers from four replicates of a 4HB producing host strain that had the genes sucAB and mutant lpdA overexpressed on pZS* compared with the corresponding averages from four replicates of the control strain. ALD and ADH were expressed on the plasmid pPZSX23R. FIG. 74 shows the reduction in the average glutamate numbers from the four replicates of the strain that had the genes sucAB and mutant lpdA overexpressed on pZS* and compared with those from the control. These results show that expression of sucAB resulted in a dramatic reduction in the amount of glutamate produced and did not adversely affect the production of BDO or 4HB.

### Example XXXIII

### Overexpression of a Non-Phosphotransferase Sugar Uptake System

This example describes overexpression of glucokinase, galP and or glf as representatives of a non-phosphotransferase (PTS) sugar uptake system. See corresponding example in WO2013/186402.

The primary mode of glucose uptake in the host strains is the PTS system. Every molecule of glucose converted into glucose-6-phosphate (G6P) via this system is accompanied by the conversion of one molecule of PEP into pyruvate. Note that PEP is converted into oxaloacetate via PEP carboxylase in the strains. Additionally, PEP is also converted into pyruvate (by the PTS system as well as pyruvate kinase) and subsequently into acetyl-CoA by pyruvate dehydrogenase in the strains. The BDO pathway requires equimolar amounts of oxaloacetate and acetyl-CoA for forming each mole of BDO. The fixed stoichiometry associated with the PTS system disturbs this balance, leading to higher ratios of acetyl-CoA relative to oxaloacetate. This then leads to production of ethanol and acetate in the cells. The overexpression of non-PTS mode of sugar uptake helps alleviate this issue and balance the amount of oxaloacetate available in comparison to acetyl-CoA, thus increasing flux through the BDO pathway, reducing the C2 byproducts, acetate and ethanol, and reducing the C3 byproducts, pyruvate and alanine.

The primary mechanism of non-PTS glucose uptake is via a permease such as galP to import the sugar and then its ATP-dependent phosphorylation via the kinase encoded by glk. An alternate way to uptake glucose is via the glucose facilitator, glf. While E. coli does not have this facilitator, one was cloned from Zymomonas mobilis and introduced into E. coli (Parker et al. Mol Microbiol. 15(5):795-802 (1995)).

### Example XXXIV

### Expression of Gamma-butyrolactone Esterase

This example describes expression of a gamma-butyrolactone esterase. See corresponding example in WO2013/186402.

Gamma-butyrolactone (GBL) is a byproduct formed during the fermentation of sugars to 1,4-butanediol (BDO). It is formed from the unstable pathway intermediate 4-hydroxybutyryl-CoA. To a small extent, it can also formed by spontaneous lactonization of 4-hydroxybutyrate.

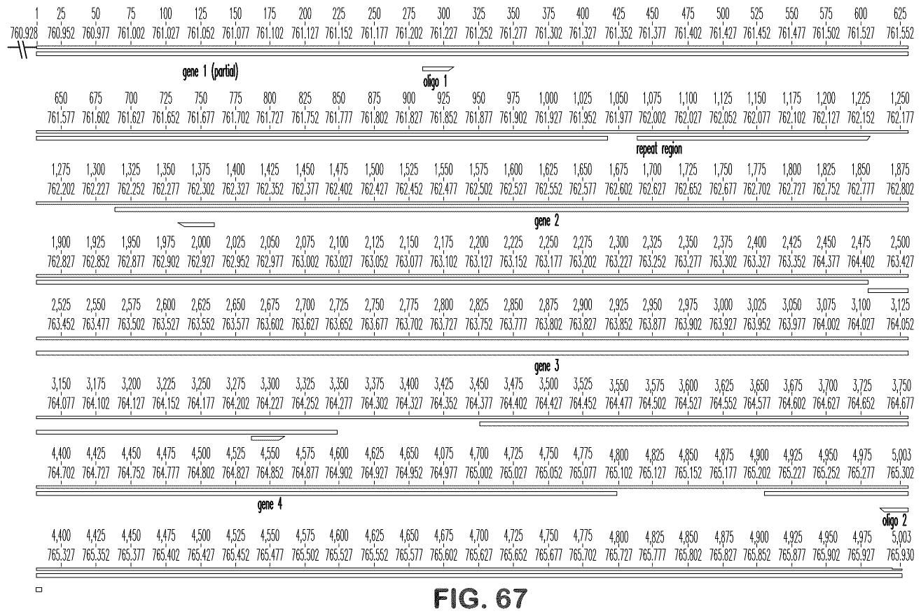

Hydroxyacids and their corresponding lactones exist in pH-dependent equilibrium with each other. Lactonization is acid-catalyzed and favorable at low pH. Under alkaline conditions the equilibrium is driven to the open-chain hydroxycarboxylate anion. At the cytoplasmic pH, typically 7.4, hydrolysis of the GBL lactone is favored (FIG. 75). The hydrolysis of GBL to 4-HB can be accelerated in the presence of an enzyme with GBL esterase activity, thus improving the yield and eliminating a byproduct that can complicate downstream separations of the product.

Esterases from Yersinia intermedia 29909 and Agrobacterium tumefaciens str. C58 (Carlier et al., Mol. Plant Microbe Interact. 17(9):951-957 (2004)) were identified and selected as exemplary esterase genes. The nucleotide sequence of the Yersinia gene (locus yinte0001_13710) is shown in FIG. 89 (see also GenBank ZP_04636075.1; GI:238792441). The nucleotide sequence of the gene from Agrobacterium tumefaciens is shown in FIG. 90. The gene can also be designated ahlK. Additionally exemplary esterases include AttM of Escherichia coli KTE10 (GenBank ZP_19612688.1, GI:432369596) and attM of Photorhabdus asymbiotica (GenBank YP_003039319.1 GI:253987963).

Table 41 shows BDO, 4HB and GBL data from 96 well-plates of four replicates of a strain that had the esterase integrated (Strain 2387) and compared with the control with no esterase (strain 2237). Host strain 2237 is based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). Strain 2237 contains deletions in the native alcohol dehydrogenase genes adhP, yqhD, yahK, and yjgB. It also contains a chromosomally integrated copy of 4-hydroxybutyryl-CoA transferase expressed from the pA promoter at the attB locus. An additional deletion in poxB was also introduced.

TABLE 41
BDO, 4HB and GBL data from 96 well-plates of four replicates of a
strain that had the esterase integrated (Strain 2387) and compared with
the control with no esterase (strain 2237). All concentrations are in
mM and were measured after 19 hours of culture time.

| Host | OD | 4HB | BDO | GBL |
|---|---|---|---|---|
| 2237 | 3.56 | 2.50 | 27.70 | 4.54 |
| 2237 | 3.43 | 2.54 | 32.20 | 5.00 |
| 2237 | 3.37 | 2.44 | 27.10 | 4.34 |
| 2237 | 3.54 | 2.19 | 22.60 | 3.26 |
| 2387 | 3.80 | 0.00 | 23.60 | 0.00 |
| 2387 | 3.87 | 2.59 | 24.70 | 0.65 |
| 2387 | 4.00 | 2.62 | 27.70 | 0.59 |
| 2387 | 4.30 | 3.20 | 29.00 | 0.68 |

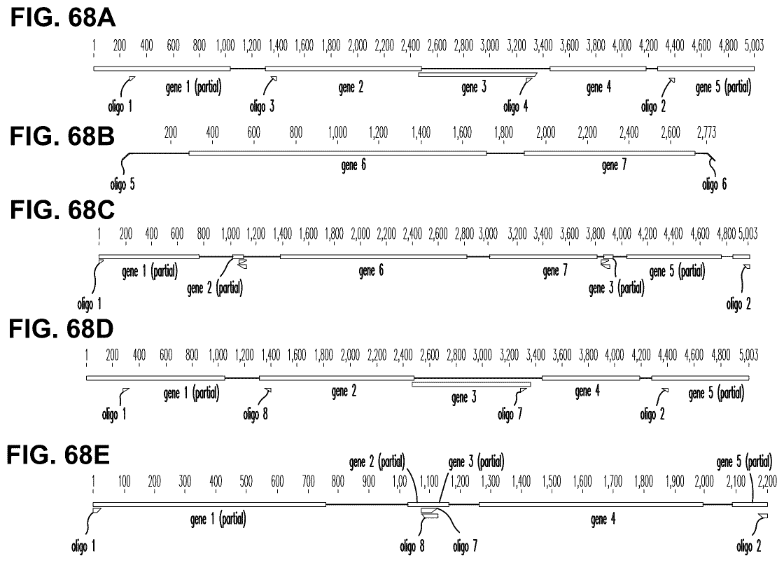

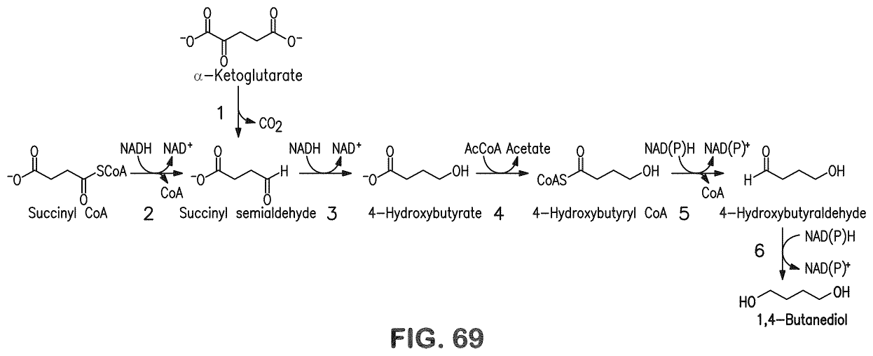

FIG. 76 shows the average BDO and 4HB numbers from four replicates of a host strain capable of producing 4-hydrobutyryl-CoA that had the esterase integrated (2387) compared with the corresponding averages from four replicates of the control strain (2237). FIG. 77 shows the average GBL numbers from four replicates of a host strain that had the esterase integrated (2387) compared with the corresponding averages from four replicates of the control strain (2237). Lower GBL was observed with the strains that had the esterase integrated. These results demonstrate expression of an enzyme having gamma-butyrolactone esterase activity without adversely affecting production of BDO or 4HB.

### Example XXXV

### Deletion of Succinyl-CoA Synthetase

This example describes deletion of succinyl-CoA synthetase. See corresponding example in WO2013/186402.

Succinyl-CoA synthetase is encoded by sucCD in Escherichia coli. The deletion of arcA highly upregulates the expression of these genes (and the entire sdh-suc operon) in the cells. Repeated rounds of flux through the TCA cycle leads to carbon loss as CO₂. The deletion of sucCD blocked the TCA cycle downstream of succinyl-CoA, reducing the CO₂ losses by ˜70% and leading to a corresponding increase in BDO titers and yields.

The deletion of sucCD was introduced in host strain 4070. This strain is based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). Strain 4070 contains deletions in the native alcohol dehydrogenase genes adhP, yqhD, yahK, and yjgB. Additionally, this strain contains a chromosomally integrated copy of 4-hydroxybutyryl-CoA transferase expressed from the p119 promoter at the attB locus. It also has deletions of cytochrome oxidases, cyoABCD and appBC. The adh was integrated on the chromosome under the promoter p119 at the hemN locus.

TABLE 42
BDO and 4HB data from 96 well-plates of four replicates of a 4-HB
producing strain that had the sucCD genes deleted (Strain 4269)
compared with the no deletion control (strain 4070). Higher 4HB was
observed with strains that had the sucCD deleted. All concentrations
are in mM and were measured after 24 hours of culture time.

| Host | OD | 4HB | BDO | |
|---|---|---|---|---|
| 4070 | 3.62 | 0.289 | 37.2 | |
| 4070 | 3.58 | 0.777 | 41.4 | |
| 4070 | 3.33 | 0.377 | 28.5 | |
| 4070 | 3.75 | 0.792 | 54.4 | |
| 4269 | 2.95 | 8.93 | 50.5 | |
| 4269 | 2.68 | 8.07 | 50.5 | |
| 4269 | 3.34 | 8.65 | 50.1 | |
| 4269 | 2.94 | 8.55 | 51.8 | |
| Host | plasmid_1 | OD | 4HB | BDO |
| 4070 | pZS*13S-p100-ALD | 3.62 | 0.289 | 37.2 |
| 4070 | pZS*3S-p100-ALD | 3.58 | 0.777 | 41.4 |
| 4070 | pZS*13S-p100-ALD | 3.33 | 0.377 | 28.5 |
| 4070 | pZS*13S-p100-ALD | 3.75 | 0.792 | 54.4 |
| 4269 | pZS*13S-p100-ALD | 2.95 | 8.93 | 50.5 |
| 4269 | pZS*13S-p100-ALD | 2.68 | 8.07 | 50.5 |
| 4269 | pZS*13S-p100-ALD | 3.34 | 8.65 | 50.1 |
| 4269 | pZS*13S-p100-ALD | 2.94 | 8.55 | 51.8 |

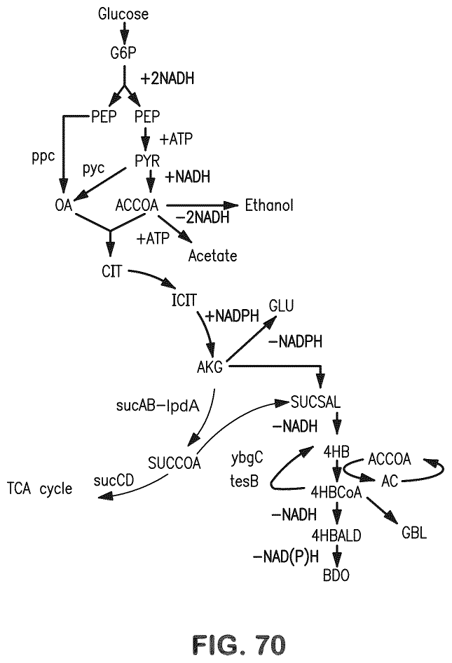

FIG. 78 shows the average BDO and 4HB numbers from four replicates of a host strain that had the genes sucCD deleted (4269) compared with the corresponding averages from four replicates of the control strain (4070). These results demonstrate increased yield of BDO and 4HB with the deletion of sucCD.

### Example XXXVI

### Deletion of Acyl CoenzymeA Thioesterases

This example describes deletion of acyl coenzymeA thioesterases ybgC and tesB. See corresponding example in WO2013/186402.

One of the intermediates in the BDO pathway is 4-hydroxybutyryl-CoA. This compound can spontaneously or enzymatically get cyclized to form GBL (gamma-butyryl-lactone). E. coli possesses some reversible CoA thioesterases reported to have broad substrate specificity on CoA substrates of C4 to C6 groups. The deletion of these gene candidates eliminated approximately 50% of the GBL formation via the pathway. Such activities can be found analogously in other organisms.

The deletions of tesB and ybgC were made in host strain 1136. This strain is based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). Strain 1136 has additional deletions in ndh (NADH dehydrogenase II) and succinate semialdehyde dehydrogenase encoding genes, sad and gabD.

TABLE 43
OD, BDO, 4HB and GBL numbers of four replicates of a strain capable
of producing 4-hydroxybutyryl-CoA that had both tesB and ybgC
deleted (1197) and compared with the control with neither
of these deletions (Host 1136). All concentrations are in mM
and were measured after 40 hours of culture time
in 20 mL bottles.

| Host | OD | BDO | 4HB | GBL |
|---|---|---|---|---|
| 1136 | 2.428 | 26.2 | 3.1 | 1.8 |
| | 2.344 | 27.4 | 2.7 | 1.7 |
| | 2.392 | 26.6 | 3.3 | 1.8 |
| 1197 | 3.324 | 29.9 | 2.8 | 1.0 |
| | 2.712 | 29.1 | 2.6 | 0.9 |
| | 2.924 | 32.0 | 2.9 | 0.9 |

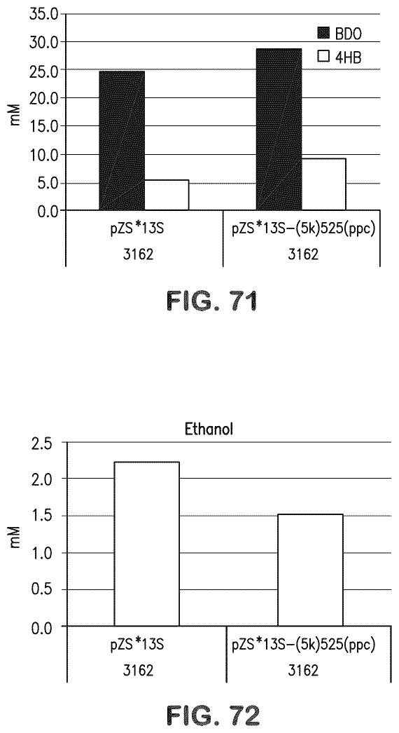

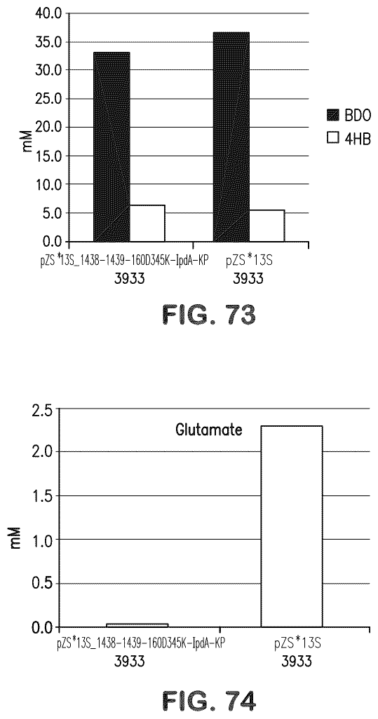

FIG. 79 shows the average BDO and 4HB numbers from three replicates of a host strain that had the genes ybgC and tesB deleted (1197) compared with the corresponding averages from three replicates of the control strain (1136). FIG. 80 shows the average GBL numbers from three replicates of a host strain that had the genes ybgC and tesB deleted (1197) compared with the corresponding averages from three replicates of the control strain (1136). These results demonstrate that deletion of one or more acyl coenzyme A thioesterases decreased production of gamma-butyrolactone, increased production of BDO and had no adverse affect on production of 44HB.

### Example XXXVII

### Deletions to Prevent or Reduce Backflux into the Pathway

This example describes deletions to prevent backflux into the pathway. See corresponding example in WO2013/186402.

At high titers of 1,4-butanediol (BDO) in the culture, it starts to get reimported into the cells. Once inside the cells, it is metabolized through the TCA cycle. To prevent this backflux of BDO into the cells, alcohol dehydrogenases that were identified as candidates for the reverse flux were deleted. The candidates were identified by two approaches, first, all the alcohol dehydrogenases were listed and prioritized based on their substrate specificity. Microarrays were conducted to determine how high each one of them was expressed. Based on this, a short list of 5 candidates was generated from a list of approximately 35 adh candidates. Secondly, protein fractions with endogenous backflux activity (NADP+ dependent BDO conversion to 4-hydroxybutyraldehyde) were isolated from strain 1427 (alcohol dehydrogenase encoded by yqhD removed and no downstream pathway present, cells accumulate 4HB but do not make BDO) grown microaerobically in MM9 media. The sample was purified while following the backflux activity. The strategy employed is outlined below.

Based on early experiments, the endogenous ADH activity was found to be primarily NADPH dependent and salted out in high (>35%) ammonium sulfate. Afresh sample of 1427 cells from a 2 L LB preparation grown aerobically overnight was lysed using the microfluidizer at 15,000 psi. The sample was then fractionated via ammonium sulfate. The 35% pellet contained little ADH activity and was not processed further, while the 60% pellet contained active ADH with significant backflux activity and was moved onto further purification.

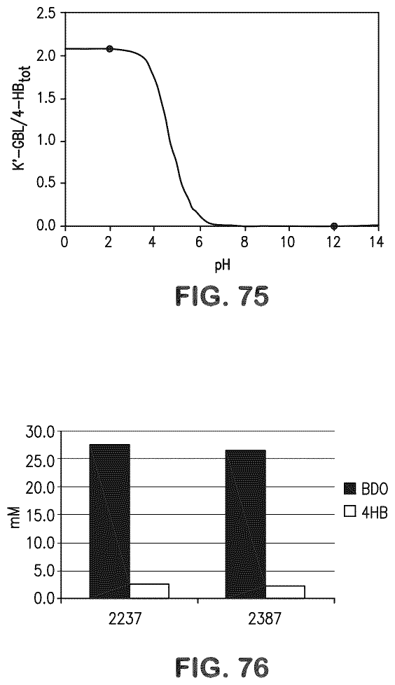

Anion Exchange Purification. The 60% ammonium sulfate pellet was resuspended in a low salt buffer and dialyzed overnight to remove the residual ammonium sulfate. The sample was loaded onto a 5 ml Q-sepharose column (strong anion exchange) and eluted via a slow increasing salt gradient. The profile for the anion exchange elution, backflux activity, as well as the SDS page for the fractions is shown below in FIG. 81.

The first large peak shown on the left is off-scale and corresponds to proteins that did not bind the column. The peak had low backflux activity. The next peak shown in the middle of the chromatogram had significant backflux activity. An SDS-PAGE of this peak was run and indicates that further purification is needed in order to isolate the protein of interest.

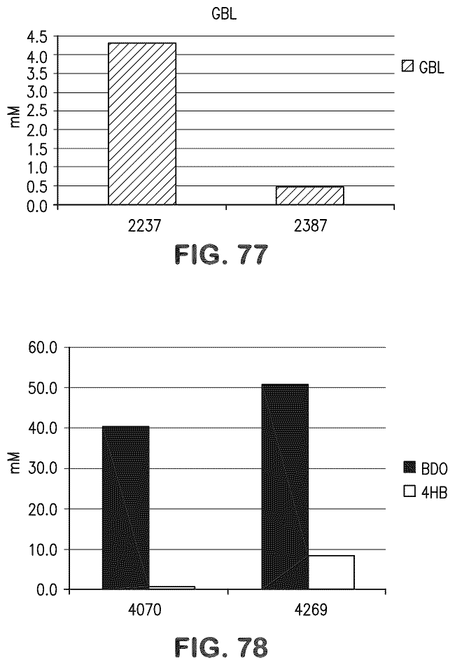

Size Exclusion Chromatography. The peak backflux activity from anion exchange was pooled and concentrated to allow for application to a S200 sephacryl size exclusion column. The column successfully removed impurities from the sample and the results are shown below (FIG. 82).

The results of the sizing column indicated that the protein of interest behaved as a mid-range MW protein, ˜50 kDa. However, it was not clear what the identity of the protein was, but it narrowed down to approximately 20 bands on the gel. Additional chromatography was performed to attempt to clean up the sample prior to mass spec analysis.

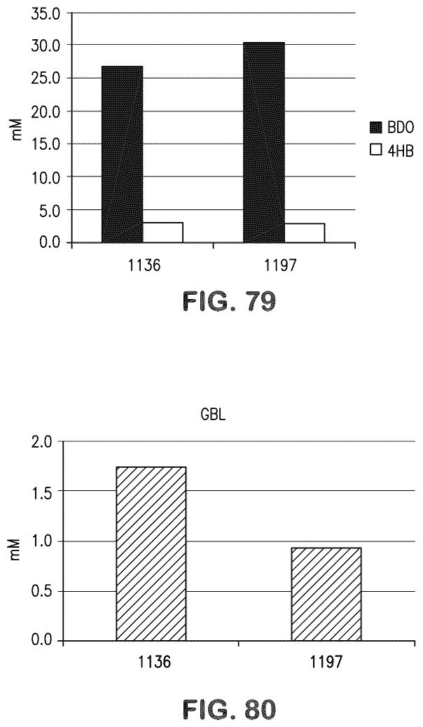

Hydrophobic Interaction Chromatography. The peak with the highest backflux activity from the size exclusion column was pooled and loaded on a phenyl sepharose column (hydrophobic interaction column). The ionic strength of the sample was increased to facilitate binding to the resin. The results are shown below (FIG. 83).

The sample shown on the SDS-PAGE was analyzed by mass spectrometry in order to identify the protein(s) that is responsible for the backflux. From the gel there are 5 prominent bands alongside a small handful of background proteins (˜5).

The purified sample was analyzed by mass spectrometry analysis, and a list of 30 proteins was obtained. From this list the gene selected was:

| Accession | Name | Gene |
|---|---|---|
| gi\|170083708 | Zn-dependent/NAD(P)-binding | yjgB |
| | alcohol dehydrogenase | |

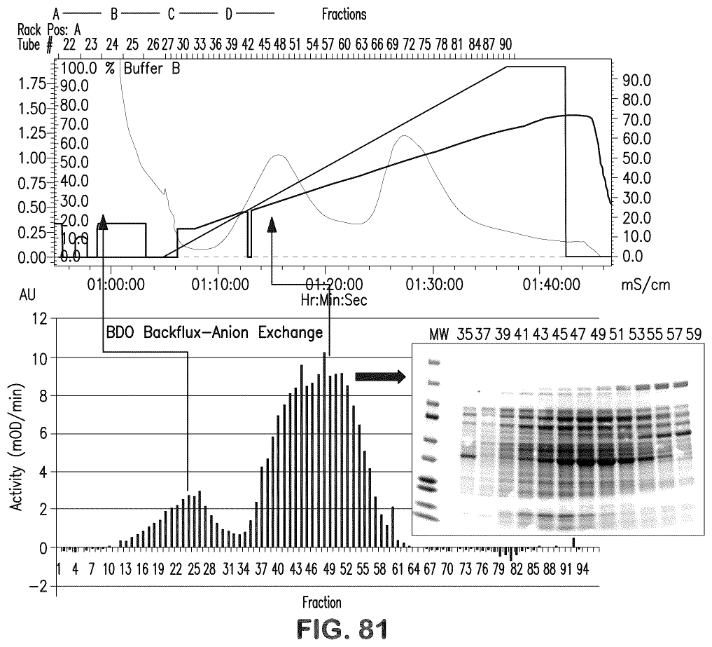

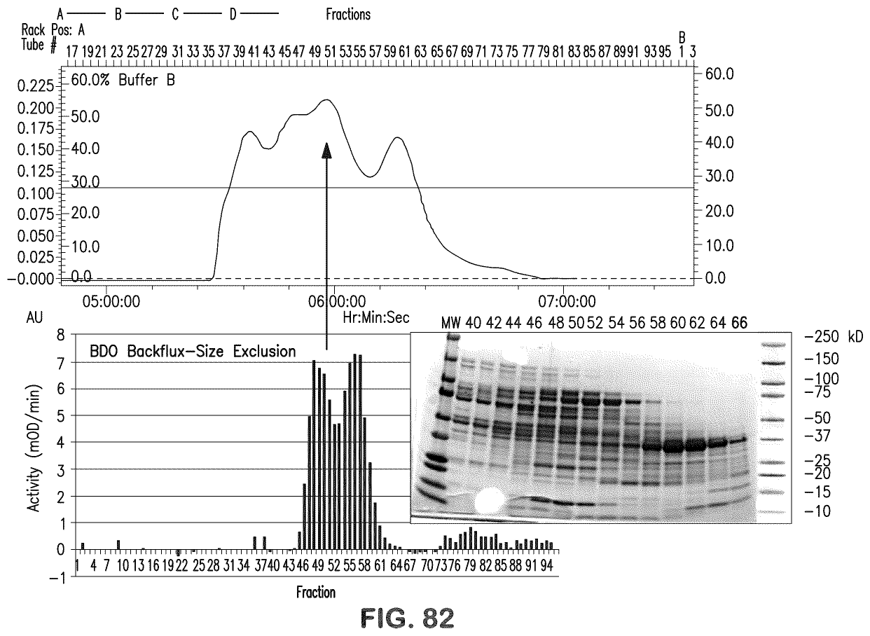

The yjgB gene was cloned with a streptavidin tag, expressed and purified in order to characterize its properties. The results of the purification, as analyzed by SDS-PAGE, are shown in FIG. 84. The protein expressed well, and acceptable yields of purified protein were obtained. Initial characterization with BDO as a substrate was performed in the presence of NADP+ and robust activity was confirmed (see FIG. 85).

On the basis of the protein fractionation and the microarray results, four genes were selected and deleted on top of each other to obtain strain 1889 from 1872. The genes were yqhD (b3011; GenBank NP_417484.1, GI:16130909), yjgB (b4269; GenBank NP_418690.4, GI:90111716), yahK (b0325; GenBank NP_414859.1, GI:16128310) and adhP (b1478; GenBank NP_415995.4, GI:90111280).

The deletions of these four alcohol dehydrogenases were introduced in strain 1872. This strain is based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). Strain 1872 contains a chromosomally integrated copy of 4-hydroxybutyryl-CoA transferase expressed from the pA promoter at the attB locus.

TABLE 44
OD, BDO, 4HB and GBL numbers of four replicates of a strain that
had all four alcohol dehydrogenases identified for carrying backflux
deleted (1889) compared with the control with neither of these
deletions (Host 1872). The gene cat2 for 4HB conversion into
4-hydroxybutyryl-CoA was expressed on F′ whereas ALD and ADH
were expressed on pZS *-13S as described already.
All concentrations are in mM and were measured
after 24 hours of culture time in 96 well-plates.

| Host | OD | 4HB | BDO | GBL |
|---|---|---|---|---|
| 1872 | 4.61 | 3.3 | 60.8 | 12.5 |
| 1872 | 6.09 | 3.6 | 90.2 | 15.3 |
| 1872 | 5.88 | 3.1 | 84.4 | 17.3 |
| 1872 | 5.84 | 2.9 | 96.3 | 15.9 |
| 1889 | 5.94 | 3.6 | 92.6 | 14.0 |
| 1889 | 6.59 | 3.3 | 88.9 | 13.1 |
| 1889 | 6.59 | 3.3 | 82.4 | 13.4 |
| 1889 | 6.58 | 3.6 | 89.4 | 14.4 |

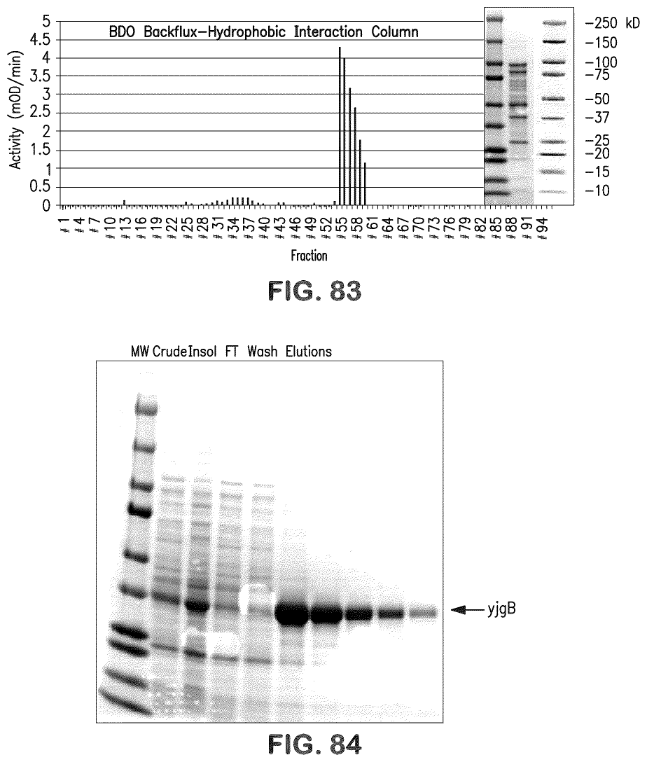

FIG. 86 shows the average BDO, 4HB and GBL numbers for the strain 1872 versus strain 1889. These results demonstrate that metabolic modifications that decrease backflux result in an increased yield of BDO and no adverse affect on production of 4HB.

### Example XXXVII

### Deletions to Improve the Energetic Efficiency of Oxidative Phosphorylation

This example describes deletions to improve the energetic efficiency of oxidative phosphorylation. See corresponding example in WO2013/186402.

The electron transport chain of Escherichia coli has multiple NADH dehydrogenases and cytochrome oxidases, with varying ability to translocate protons. For example, NADH dehydrogenase II in E. coli is an NADH consuming system that is not linked with proton translocation (H+/2e-=0) whereas NADH dehydrogenase I encoded by nuo is reported to translocate 4 protons per pair of electrons. The major role of Ndh-II is to oxidize NADH and to feed electrons into the respiratory chain (Yun et al., J. Appl. Microbiol. 99:1404-1412(2005)). The affinity of NdhII for NADH is relatively low (Hayashi et al., Biochim. Biophys. Acta 977:62-69 (1989)). It has been suggested that NdhII may operate to regulate the NADH pool independently of energy generation and is likely to be important when the capacity of bacteria to generate energy exceeds demand. The ndh gene has been shown to be repressed by the fnr gene product in such a way that the expression is optimal under conditions of high oxygen concentrations. The deletion of ndh would thus help in improving the energy efficiency of the cell. Similarly, there are several other NADH dehydrogenases that are not known to translocate any protons and thus do not help in ATP production, for example, wrbA, yieF, and kefF in E. coli. Homologues of these can be found in other organisms and eliminated to improve the ATP production for every unit of oxygen consumed.

Host strain 879 (the parent strain for ndh deletion) was based on strain ECKh-432 whose construction is described above and in US20110045575. Notable modifications include deletions in adhE, ldhA, pflB, mdh, and arcA. Strain ECKh-432 also contains chromosomally integrated copies of sucD from Porphyromonas gingivalis (encoding CoA-dependant succinate semialdehyde dehydrogenase), sucA from Mycobacterium bovis (encoding alpha-ketoglutarate decarboxylase), 4hbd from P. gingivalis (encoding 4-hydroxybutyrate dehydrogenase), and 4hbd from Clostridium kluyveri (encoding 4-hydroxybutyrate dehydrogenase). Strain 879 also has deletions of the succinate semialdehyde dehydrogenase genes, sad and gabD.

TABLE 45
The BDO, 4HB and GBL production in three replicates of the strain
that had ndh deleted compared with the replicates of the host strain (879)
with ndh intact. The experiments were conducted in 20 mL bottles
as described in the protocol. The ALD and ADH were present
on the plasmid pZS* to allow the conversion of 4-HB CoA to
4-HB aldehyde and subsequently to BDO. All concentrations
are in mM and were measured after 24 hours of culture time.

| Host | OD | BDO | GBL | 4HB |
|---|---|---|---|---|
| 879 | 1.61 | 3.27 | 0.83 | 1.28 |
| 879 | 2.45 | 4.15 | 0.99 | 1.14 |
| 956 | 2.31 | 4.48 | 0.63 | 1.01 |
| 956 | 2.01 | 4.31 | 0.59 | 0.96 |
| 956 | 2.54 | 4.92 | 0.59 | 1.00 |

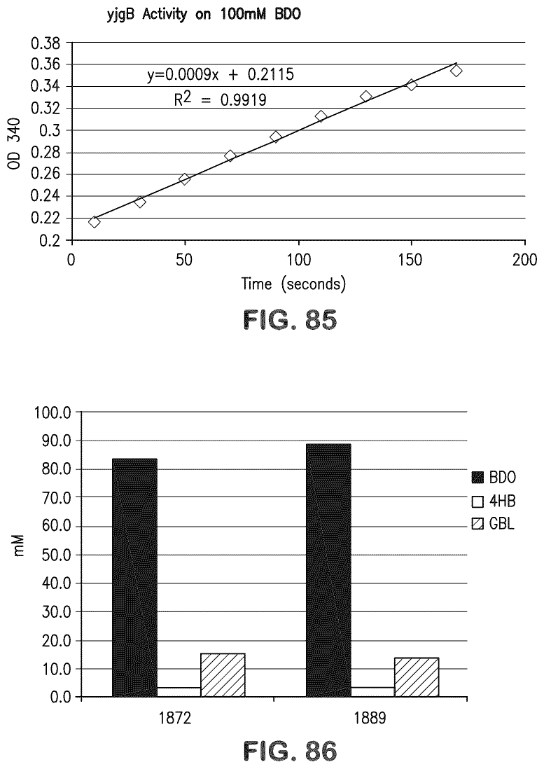

FIG. 87 shows the average BDO, 4HB and GBL numbers for the strain 956 versus strain 879. These results demonstrate that deletions that improve the energy efficiency of oxidative phosphorylation increase the yield of BDO and have no adverse affect on 4HB production.

### Example XXXIX

### Deletion of Cytochrome Oxidases

This example describes deletion of cytochrome oxidases. See corresponding example in WO2013/186402.

On the electron output side of the electron transport chain, multiple cytochrome oxidases are present that have different energy-conserving efficiencies. The cytochrome bo complex, encoded by the cyo operon, actively pumps electrons over the membrane and results in an H+/2e-stoichiometry of 4. The cytochrome bd-I complex does not actively pump protons, but due to the oxidation of the quinol on the periplasmic side of the membrane and subsequent uptake of protons from the cytoplasmic side of the membrane, which are used in the formation of water, the net electron transfer results in a H+/2e-stoichiometry of 2. This is encoded by the cyd operon. Until recently, the proton translocation stoichiometry of cytochrome bd-II oxidase, encoded by appBC, was not known, but it has now been established that this oxidase is electrogenic (Borisov et. al., Proc. Natl. Acad. Sci. USA 108:17320-17324 (2011)). These genes are normally induced upon entry into stationary phase or under conditions of carbon and phosphate starvation (Atlung et al., J. Bacteriol. 179:2141-2146 (1997)).

Properties and abundance of the cytochrome oxidases: The cyd operon is regulated in an oxygen-dependent manner by fnr and arcA and the expression is optimal under microaerophilic conditions (Calhoun et al., J. Bacteriol. 175:3020-3025 (1993)). The bo-type oxidase predominates when E. coli is grown at high oxygen tension. The cyo operon is also regulated by both arcA and fnr. A literature study indicates that in aerobically grown cells the cytochrome o oxidase is present at a level of approximately 304 molecules per cell and the cytochrome d oxidase at 204 molecules per cell (Minohara et al., J. Biosci. Bioengineer 93(5):464-469 (2002)). Under anaerobic conditions, cytochrome d oxidase is predominant and is present at about 606 molecules per cell in contrast to only 2 molecules per cell of cytochrome o oxidase. The Km for O₂ of cytochrome bd oxidase is much lower than that of the cytochrome bo oxidase (Minohara et al, supra, 2002). The former has a Km of 10 nM, and cytochrome bo oxidase has a Km of ˜1 μM.

TABLE 46
Enzymatic characteristics of the cytochrome-bd oxidases from E. coli
(from Bekker et al., J. Bacteriol. 191:5510-5517 (2009)) ).

| | | | Kₘ | | In vivo |
|---|---|---|---|---|---|
| | Vₘₐₓ (mol | Kₘ | (UQ-H₂) | Cellular | sp act (mol |
| | O/mol | (O₂) at | at | content | O₂/mol |
| | cytochrome | pH 7 | pH 7 | (nmol/g | cytochrome |
| Cytochrome | bd/s) | (μM) | (μM) | protein) | bd/s) |
| Cytochrome bd-I | 218 ± 20 | 0.3 | 85 ± 5 | 90 ± 29 | 58 ± 11 |
| Cytochrome bd-II | 818 ± 75 | 2.0 ± 0.3 | 250 ± 45 | 91 ± 32 | 70 ± 12 |
| Cytochrome bo | 225 | 6.0 | 47 | ND | ND |

In a study attempting to determine the P/O ratios in aerobically-grown E. coli (Noguchi et al., J. Biochem. 136:509-515 (2004)), it was determined that NDH-1 formed the predominant NADH dehydrogenase under both standard feed and limiting feed of glucose. However, cytochrome bd oxidase was found to be present in higher concentrations than cytochrome bo oxidase. The following table illustrates their findings on various NADH dehydrogenases and cytochrome oxidases.

TABLE 47
Concentrations of NADH dehydrogenases and cytochrome oxidase under aerobic growth
conditions with different glucose feeding strategies (from Noguchi et al., supra, 2004).

| | NDH-1 | NDH-2 | bo-type | bd-type | NDH-1/Total | bo-type/Total |
|---|---|---|---|---|---|---|
| Conditions (Phase) | (μmol/s/mg of protein) | | | | (%) | (%) |
| Standard glucose | | | | | | |
| (exponential: 3 h) | 1.58 ± 0.07 | 1.11 ± 0.06 | 1.38 ± 0.12 | 1.01 ± 0.10 | 58.8 ± 2.4 | 57.9 ± 4.5 |
| (exponential: 5 h) | 1.35 ± 0.03 | 0.86 ± 0.03 | 0.70 ± 0.05 | 1.07 ± 0.04 | 61.0 ± 1.4 | 39.6 ± 2.6 |
| (early stationary: 8 h) | 1.17 ± 0.01 | 0.88 ± 0.01 | 0.46 ± 0.03 | 0.98 ± 0.02 | 57.1 ± 0.5 | 32.0 ± 1.9 |
| Limited glucose | | | | | | |
| (early stationary) | 0.97 ± 0.05 | 0.70 ± 0.03 | 0.42 ± 0.04 | 0.75 ± 0.05 | 58.1 ± 2.29 | 36.0 ± 3.7 |

Effects of deleting cytochrome oxidases: A chemostat study has been reported for understanding the ratios of specific oxygen consumption rates for strains lacking different components of the electron transport chain between dilution rates of 0.1 and 0.7/hr (Calhoun et al., supra, 1993). Strains that were constrained to use the bd-type oxidase had increased oxygen consumption rates as compared to those that used only the cytochrome bo oxidase by a factor of 1.45-1.13. The ratio of the oxygen consumption rates for a strain that had both ndh and cyo deleted as compared to the strain with only ndh deleted was between 1.46 and 1.39. The authors of Calhoun et al., supra, 1993, concluded that the bd-type oxidase is less efficient than the bo-type oxidase but is still coupled.

In yet another study, it has been reported that E. coli wild-type cells containing both cytochrome bo and bd type terminal oxidases, pumped protons with a H+/O ratio of 4.5-4.9, but mutants with cytochrome bo oxidase deleted, showed ratios of 3.5-4.1 and the mutants with cytochrome bd oxidase showed a ratio of 4.8-5.6 (Minohara et al., supra, 2002). Mutants which lacked both cyo and cyd operon could not grow under aerobic conditions, but those that overexpressed cytochrome bo oxidase and those that overexpressed cytochrome bd oxidase grew as well as the parental W3110 strains. The cell yield of each of these strains was in proportion to their H+/O ratios. Interestingly, the strain which had cydAB deleted showed very poor cell yield initially. The cell yields were, however, improved dramatically once the cytochrome bo oxidase was overexpressed. The authors Minohara et al., supra, 2002) had hypothesized that the lower cell yields in the cyd mutant strain may be caused by the high H+ permeability of the membrane in the cyd mutant. They believed that the amounts of cytochrome bo in the mutant cells lacking the high-affinity oxygen-reducing cytochrome bd may not be sufficient to reduce the oxygen molecules at a low concentration, and thus the cell membranes were injured by reactive oxygen species and were made partially permeable. This cell stress could be relieved by overexpressing cytochrome bo oxidase (Minohara et al., supra, 2002).

TABLE 48
Properties of various E. coli mutants grown in a glucose-limited chemostat culture at a dilution rate
of 0.15/hr. The cytochrome shown in parentheses is the active cytochrome oxidase. Each of these
strains also had nuo deleted (from Bekker et al., supra, 2009).

| | qₒ₂ {mmol | q[_{glucose} {mmol | Y_{glucose} | q_{acetate} {mmol | q_{ATP} {mmol | |
|---|---|---|---|---|---|---|
| | (g [dry wt])⁻¹ | (g [dry wt])⁻¹ | (g [dry wt]) | (g [dry wt])⁻¹ | (g [dry wt])⁻¹ | |
| Mutant | h⁻¹} | h⁻¹} | (g Glc)⁻¹ | h⁻¹} | h⁻¹} | H⁺/2e⁻ |
| MB30 (cytochrome bd-I) | 8.8 ± 1.3 | 3.2 | 0.26 | 1.5 ± 0.5 | 16.3 ± 2.2 | 2 |
| MB37 (cytochrome bd-II) | 11.1 ± 0.6 | 6.0 | 0.14 | 5.7 ± 0.7 | 16.6 | 0.2 ± 0.1 |
| MB34 (cytochrome bo) | 6.4 ± 0.4 | 2.3 | 0.36 | 0.4 ± 0.4 | 17.0 ± 0.4 | 4 |

A recent paper (Bekker et al., J. Bacteriol. 191:5510-5517 (2009)) investigated the effects of deleting cytochrome bd-II oxidase. The phenotype of the mutant strain lacking the cytochrome bd-II oxidase included a smaller ubiquinone pool when grown under glucose-excess conditions. This strain also had a decreased oxygen flux in glucose-limited chemostat conditions. These results suggested that cytochrome bd-II oxidase contributed significantly to the overall respiratory electron flux. Wild type E. coli MG1655 cells had a specific respiration rate that was approximately 46% higher than that of the mutant strain that lacked appBC (8.7 vs. 5.9 mmol/gDCW.hr). These were measured in glucose-limited chemostats at a dilution rate of 0.2/hr. In order to improve the energetic efficiency of the cells, the appBC was knocked out in the strains (Host strain 2424), and additionally cyoABCD was knocked out (Strain 2471). The table below shows the data for production of the C4 metabolites in 96 well plates with each of these strains. An additional advantage of deleting the two cytochrome oxidases (encoded by appBC and cyoABCD) and ndh is that it makes the cells relatively robust to a range of oxygen transfer rates in bigger fermenters. Since cydAB has a fixed stoichiometry for proton translocation and so does nuo (NADH dehydrogenase I), the P/O ratio is rather constant and so is the biomass rate in different parts of a fermenter.

TABLE 49
The BDO, 4HB and GBL production in three replicates
of the strains 1889 and 2471. The ALD and
ADH were present on the plasmid pZS* to allow the
conversion of 4-HB CoA to 4-HB aldehyde and
subsequently to BDO. All concentrations are in mM and
were measured after 24 hours of culture time in 96-well plates.

| Host | OD | 4HB | BDO | GBL |
|---|---|---|---|---|
| 1889 | 3.63 | 5.71 | 57.20 | 4.48 |
| 1889 | 3.05 | 5.65 | 50.60 | 3.49 |
| 1889 | 3.40 | 4.14 | 51.90 | 3.90 |
| 1889 | 3.19 | 4.23 | 53.10 | 3.69 |
| 2471 | 3.56 | 5.62 | 64.20 | 4.80 |
| 2471 | 2.96 | 5.73 | 53.30 | 2.75 |
| 2471 | 3.07 | 5.39 | 42.80 | 2.67 |
| 2471 | 3.11 | 4.01 | 45.90 | 2.66 |

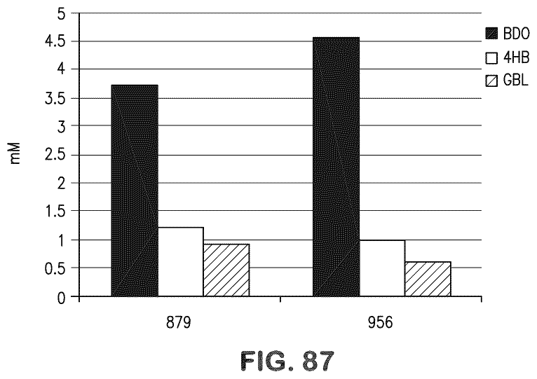

FIG. 88 shows the average BDO, 4HB and GBL numbers of the strains, 1889 (see above) and 2471. These results demonstrate that deletion of cytochrome oxidases results in deletion of appBC resulted in a decrease in BDO and 4HB production, although both BDO and 4HB are still produced in good quantities, and these cells are expected to exhibit improved energy efficiency that may be advantageous in scaled up fermentation conditions.

The results disclosed herein demonstrate high yield of desired products such as 4-HB, 4-HBal, 4-HBCoA, BDO or putrescine, in particular BDO. Such modifications have been demonstrated to increase product yield of BDO. Such beneficial modifications, including both increasing and decreasing expression of enzymes, resulted in decreased CO₂ loss, decreased by-product formation such as ethanol, acetate, pyruvate, alanine, glutamate and GBL. Additional modifications decreased backflux from desired products such as BDO into pathway intermediates or precursors such as those in the TCA cycle. Additional improvements have been made by identifying enzymes that more efficiently cany out a desired pathway reaction and optionally subjecting the enzyme to evolution to generate variants having improved characteristics such as increased substrate to product conversion, stability, and the like. Such enzymes have included cat2, aldehyde dehydrogenase and aldehyde dehydrogenase, as disclosed herein. In addition, various promoters have been tested to identify promoter variants that provide more efficient transcription of a gene. Additionally, as disclosed herein, various backflux and by-product reactions can decrease the efficiency of product formation in a desired pathway. In some cases, product yield can be increased by decreasing expression of a pathway enzyme if higher expression of the pathway enzyme results in an increase in by-product formation. For example, such a result has been observed with aldehyde dehydrogenase, in which lower expression reduces ethanol production without lowering BDO production.

### Example XXXX

### Overexpression of Acetate Kinase and Phosphotransacetylase

This example describes overexpression of acetate kinase and phosphotransacetylase which improves the production of 4HB and BDO.

ackA refers to a gene that encodes for acetate kinase activity. pta refers to a gene that encodes for phosphotransacetylase activity. The net reaction catalyzed by both enzymes involves the conversion of acetate, ATP, and CoA into acetyl-CoA, ADP and phosphate. The overexpression of acetate kinase and phosphotransacetylase leads to increased conversion of acetate to acetyl-CoA, thus reducing the secretion of acetate and increasing the production of 4hb and BDO. The E. coli ackA (NP_416799.1, GI:16130231) and pta (NP_416800.1, GI:16130232) genes were cloned on the plasmid pZS*13S under control of the weak constitutive promoter, p107. Expression of ackA and pta in such strains is thus increased over native levels.

Table 50 shows BDO, 4HB, and acetate production in a 96-well plate from the strain 4539 containing either an empty plasmid, pZS*13S, or containing a plasmid, pZS*13S-p107-301-302, that expresses ackA (301) and pta (302). The host strain 4539 is based on strain 3162 whose construction is described above. Strain 4539 contains chromosomally integrated copies of 4-hydroxybutyryl-CoA reductase (ALD), 4-hydroxybutyraldehyde reductase (ADH) and 4-hydroxybutyryl-CoA transferase expressed from constitutive promoters. The genes used for these enzyme steps are described herein. The ADH was integrated on the chromosome under the promoter p119 at the hemN locus. The ALD was integrated on the chromosome under the promoter p119 at the aspA locus. The 4-hydroxybutyryl-CoA transferase was integrated on the chromosome at the attB locus under the p119 promoter. Strain 4539 additionally contains deletions in succinyl-CoA synthetase (sucCD) and a prophage integration site (ycil). In addition, the native promoter of ppc was replaced with a stronger constitutive promoter. Strain 4539 also contains an inactivated arcA gene. Table 50 shows that expression of acetate kinase and phosphotransacetylase enhances 4HB and/or BDO production while reducing acetate production in strain 4539 versus the controls denoted by “pZS*13S”. Cells were cultured using the 96 well-plate protocol described previously above.

TABLE 50
BDO, 4HB, and acetate production of E. coli
host strain 4539 containing either the plasmid
construct denoted by “pZS*13S” which is empty or the
plasmid construct denoted by “pZS*13S-p107-301-
302” which expresses both ackA (301) and pta (302).
BDO, 4HB, and acetate concentrations are in mM.

| Host | Plasmid #1 | OD | 4HB | BDO | Acetate |
|---|---|---|---|---|---|
| 4539 | pZS*13S-p107-301-302 | 3.04 | 10.90 | 63.70 | 0.21 |
| 4539 | pZS*13S-p107-301-302 | 2.69 | 11.30 | 62.00 | Not measured |
| 4539 | pZS*13S-p107-301-302 | 2.56 | 10.40 | 61.60 | Not measured |
| 4539 | pZS*13S-p107-301-302 | 2.78 | 9.74 | 62.00 | Not measured |
| 4539 | pZS*13S-p107-301-302 | 2.99 | 12.20 | 61.50 | 1.00 |
| 4539 | pZS*13S-p107-301-302 | 2.85 | 11.90 | 62.10 | Not measured |
| 4539 | pZS*13S-p107-301-302 | 2.81 | 10.70 | 62.20 | Not measured |
| 4539 | pZS*13S-p107-301-302 | 2.95 | 12.60 | 64.10 | Not measured |
| 4539 | pZS*13S | 2.33 | 11.50 | 31.50 | 1.98 |
| 4539 | pZS*13S | 1.18 | 6.86 | 20.00 | Not measured |
| 4539 | pZS*13S | 1.41 | 7.17 | 21.60 | Not measured |
| 4539 | pZS*13S | 1.92 | 11.50 | 30.10 | Not measured |

### Example XXXXI

### Inactivation of arcA

This example describes the inactivation of the global regulator, arcA, which improves the production of 4HB and BDO.

An arcA mutation was reported to abolish function (luchi et al., Proc. Natl. Acad. Sci. USA 85:1888-1892 (1988)). It was later characterized (Silverman et al., J Bacteriol. 173(18):5648-5652 (1991)) and found to have an 8 amino acid insertion near the N-terminus of the protein (see sequence below). To test the effect of this arcA mutation, we restored the mutant and wild-type versions of arcA in strain 3162, described above and which contains a deletion in arcA, to create strains 3353 and 3511, respectively. In both strains, the ALD and ADH were expressed in E. coli from the pZS*13S plasmid to allow a functional pathway from 4-hydroxybutyryl-CoA to 1,4-butanediol. Table 51 shows that 4HB and BDO production are higher in strain 3353, which contains the inactivated arcA gene, compared to strain 3511, which contains the wild-type arcA gene. Cells were cultured using the 96 well-plate protocol described previously.

TABLE 51
BDO, 4HB, and glutamate production of E. coli
host strains 3353 (which contains an inactivated
arcA) and 3511 (which contains wild-type arcA).
BDO, 4HB, and acetate concentrations are in mM.

| Host | Plasmid | OD | 4HB | BDO | Glu |
|---|---|---|---|---|---|
| 3353 | pZS*13S-ALD-ADH | 3.8 | 0.77 | 57.3 | 0.00 |
| 3353 | pZS*13S-ALD-ADH | 3.8 | 0.77 | 54.6 | 0.00 |
| 3353 | pZS*13S-ALD-ADH | 3.9 | 0.78 | 60.0 | 0.00 |
| 3353 | pZS*13S-ALD-ADH | 3.7 | 0.89 | 63.1 | 0.00 |
| 3511 | pZS*13S-ALD-ADH | 3.1 | 0.28 | 17.1 | 0.45 |
| 3511 | pZS*13S-ALD-ADH | 3.0 | 0.35 | 17.5 | 0.43 |
| 3511 | pZS*13S-ALD-ADH | 2.7 | 0.20 | 10.8 | 0.44 |
| 3511 | pZS*13S-ALD-ADH | 3.1 | 0.03 | 11.7 | 0.46 |

The method for restoration of the mutant arcA gene is described below. All cloning for the insertion or deletion of genes in the chromosome of E. coli were based on the utilization of the sacB gene from Bacillus subtilis (ncbi.nlm.nih.gov/pubmed?term=6402497). The vector used is pRE118 (ATCC87693) deleted of the oriT and IS sequences. The resulting vector (3.6 kb in size and carrying the kanamycin resistance gene) was sequenced and called pRE118-V2. All clonings of fragments were done into the restriction sites KpnI and PstI of pRE118-V2, unless otherwise indicated. All PCR amplification used genomic DNA from E. coli MG1655 (ATCC47076) as DNA template. The first integration event in the chromosome was selected on LB agar plates containing Kanamycin (25 or 50 mg/L). Clones with the correct insertion were selected for resolution. They were sub-cultured twice in plain liquid LB at the desired temperature and serial dilutions were plated on LB-no salt-sucrose 10% plates. Clones that grew on sucrose containing plates were screened for the loss of the kanamycin resistance gene on LB-low salt agar medium and the deletion/insertion of the fragment of interest was verified by PCR and sequencing of the encompassing region.

The E. coli arcA gene was amplified by PCR using genomic DNA form E. coli MG1655 and primers primers ArcA-up-KpnI (5′-tattattatggtaccaatatcatgcagcaaacggtgcaacattgccg-3′; SEQ ID NO:34) and ArcA-down-PstI (5′-ataaaaccctgcagcggaaacgaagttttatccatttttggttacctg-3′; SEQ ID NO:36). The fragment was subsequently digested with the restriction enzymes KpnI and PstI, then ligated into the pRE118-V2 plasmid digested with PstI and KpnI, leading to plasmid pRE118-arcA. The nucleotide sequence of the arcA fragment was verified. An 8 amino acid insertion EGYDVFEA (SEQ ID NO:181) was created by site-directed mutagenesis using primers arcA-8aa-mut-frw 5′-ATGATGTTTTCGAAGCGGAAGGCTATGATGTTTTCGAAGCGACAGATGGCGCGGAAATGCA TCAGATCC-3′ (SEQ ID NO:182) and arcA-8aa-mut-rev 5′-TTTCCGCGCCATCTGTCGCTTCGAAAACATCATAGCCTTCCGCTTCGAAAACATCATAGCCT TCCGCTTCG-3′ (SEQ ID NO:183). This 8 AA insertion was located as described in Silverman et al., J. Bact. (173) 18:5648-5652(1991)).

pRE118-arcA* was introduced into electro-competent cells of E. coli. After integration and resolution on LB-no salt-sucrose plates as described above, the sequence of the arcA region in the chromosome was verified by sequencing using primers arcA-diag-up2 5′-taatgatatttcgcccgactgcgc-3′ (SEQ ID NO:184) and arcA-diag2-down 5′-aatgaacggtatttatttgcaagcgg-3′ (SEQ ID NO:185) and is as follows (the nucleotide sequence of the wild-type arcA gene is underlined, and the added fragment shown in bold):

(SEQ ID NO: 186)
TAATGATATTTCGCCCGACTGCGCGCAGTGGTGGCGACAGTGAAATCGAC
ATCGTGTAACGATTCAGCCAATGTCGGGAAAACTTTAATATTATCAATAA
TATCACCAGATCCATGTGCGACCCAGCGGGTGGCTGGCTCCAGGTGTGCC
TGACTATCGACAATCCGCAGATCGCTAAACCCCATCGTTTTCATTGCCCG
CGCCGCTGCCCCAATATTTTCTGCTCTGGCGGGTGCGACCAGAATAATCG
TTATACGCATATTGCCACTCTTCTTGATCAAATAACCGCGAACCGGGTGA
TCACTGTCAACTTATTACGCGGTGCGAATTTACAAATTCTTAACGTAAGT
CGCAGAAAAAGCCCTTTACTTAGCTTAAAAAAGGCTAAACTATTTCCTGA
CTGTACTAACGGTTGAGTTGTTAAAAAATGCTACATATCCTTCTGTTTAC
TTAGGATAATTTTATAAAAAATAAATCTCGACAATTGGATTCACCACGTT
TATTAGTTGTATGATGCAACTAGTTGGATTATTAAAATAATGTGACGAAA
GCTAGCATTTAGATACGATGATTTCATCAAACTGTTAACGTGCTACAATT
GAACTTGATATATGTCAACGAAGCGTAGTTTTATTGGGTGTCCGGCCCCT
CTTAGCCTGTTATGTTGCTGTTAAAATGGTTAGGATGACAGCCGTTTTTG
ACACTGTCGGGTCCTGAGGGAAAGTACCCACGACCAAGCTAATGATGTTG
TTGACGTTGATGGAAAGTGCATCAAGAACGCAATTACGTACTTTAGTCAT
GTTACGCCGATCATGTTAATTTGCAGCATGCATCAGCAGGTCAGGGACTT
TTGTACTTCCTGTTTCGATTTAGTTGGCAATTTAGGTAGCAAACATGCAG
ACCCCGCACATTCTTATCGTTGAAGACGAGTTGGTAACACGCAACACGTT
GAAAAGTATTTTCGAAGCGGAAGGCTATGATGTTTTCGAAGCGGAAGGCT
ATGATGTTTTCGAAGCGACAGATGGCGCGGAAATGCATCAGATCCTCTCT
GAATATGACATCAACCTGGTGATCATGGATATCAATCTGCCGGGTAAGAA
CGGTCTTCTGTTAGCGCGTGAACTGCGCGAGCAGGCGAATGTTGCGTTGA
TGTTCCTGACTGGCCGTGACAACGAAGTCGATAAAATTCTCGGCCTCGAA
ATCGGTGCAGATGACTACATCACCAAACCGTTCAACCCGCGTGAACTGAC
GATTCGTGCACGCAACCTACTGTCCCGTACCATGAATCTGGGTACTGTCA
GCGAAGAACGTCGTAGCGTTGAAAGCTACAAGTTCAATGGTTGGGAACTG
GACATCAACAGCCGTTCGTTGATCGGCCCTGATGGCGAGCAGTACAAGCT
GCCGCGCAGCGAGTTCCGCGCCATGCTTCACTTCTGTGAAAACCCAGGCA
AAATTCAGTCCCGTGCTGAACTGCTGAAGAAAATGACCGGCCGTGAGCTG
AAACCGCACGACCGTACTGTAGACGTGACGATCCGCCGTATTCGTAAACA
TTTCGAATCTACGCCGGATACGCCGGAAATCATCGCCACCATTCACGGTG
AAGGTTATCGCTTCTGCGGTGATCTGGAAGATTAATCGGCTTTACCACCG
TCAAAAAAAACGGCGCTTTTTAGCGCCGTTTTTATTTTTCAACCTTATTT
CCAGATACGTAACTCATCGTCCGTTGTAACTTCTTTACTGGCTTTCATTT
TCGGCAGTGAAAACGCATACCAGTCGATATTACGGGTCACAAACATCATG
CCGGCCAGCGCCACCACCAGCACACTGGTTCCCAACAACAGCGCGCTATC
GGCAGAGTTGAGCAGTCCCCACATCACACCATCCAGCAACAACAGCGCGA
GGGTAAACAACATGCTGTTGCACCAACCTTTCAATACCGCTTGCAAATAA
ATACCGTTCATTA.

### Example XXXXII

### Truncation of crr

This example describes improved 4-HB and BDO production using a truncated variant of the glucose PTS permease HA domain.

To achieve higher BDO titer and yield, the rate of glucose uptake and glycolytic flux can be balanced with the rate of BDO production. One mechanism for balancing these fluxes is to alter one or more components of a glucose transporter. In this example, improved BDO production was demonstrated using a truncated variant of the glucose PTS permease component HA (EIIA[Glc]), encoded by crr. The glucose PTS system transports and simultaneously phosphorylates glucose, releasing glucose-6-phosphate into the cytoplasm and concomitantly converting phosphoenolpyruvate to pyruvate. The IIA[Glc] component mediates the transfer of the phosphoryl group from histidine protein Hpr (ptsH) to the II[Glc] (ptsG) component. A truncated variant of the crr gene was introduced into BDO producing strains. The resulting gene is called 2311A (crr*). The DNA sequence of 2311A is shown in Table 52. Strains with the crr variant exhibit reduced production of the byproducts acetate, pyruvate and glutamate, and an increase in BDO titers and yields. Without being bound by a particular theory, the truncated variant is expected to slow down glucose uptake, thereby balancing flux through glycolysis with BDO production.

To test the performance of truncated err relative to the wild type protein, a wild-type err variant was restored in the strain ECKh-5491, resulting in the strain ECKh-5717. The host strain ECKh-5491 is based on strain ECKh-432 whose construction is described above and in US20110045575. Notable modifications include in deletions in adhE, ldhA, pflB and mdh. Strain ECKh-5491 also contains chromosomally integrated copies of sucD from Porphyromonas gingivalis (encoding CoA-dependant succinate semialdehyde dehydrogenase) and 4hbd from P. gingivalis (encoding 4-hydroxybutyrate dehydrogenase). Additionally, strain 5491 contains chromosomally integrated copies of 4-hydroxybutyryl-CoA reductase (ALD), 4-hydroxybutyraldehyde reductase (ADH) and 4-hydroxybutyryl-CoA transferase. The genes for these enzyme steps are described herein. The ADH was integrated on the chromosome under the promoter p119 at the hemN locus. The ALD was integrated on the chromosome under the promoter p119 at the aspA locus. The 4-hydroxybutyryl-CoA transferase was integrated on the chromosome at the attB locus under the p119 promoter. Strain 5491 contains deletions in the native alcohol dehydrogenase genes adhP, yqhD, yahK, and yjgB. This strain also has deletions of cytochrome oxidases, cyoABCD and appBC, succinyl-CoA synthetase (sucCD), a prophage integration site (ycil) and flagellar biosynthesis genes (flhCD, motA), the predicted transporter (yebQ), aspartate-ammonia lyase (aspA), the ferrichrome/phage/antibiotic outer membrane porin (fhuA), glutamate synthase (gltBD), a low specificity threonine aldolase (ItaE), PEP carboxykinase (pckA), the glycine cleavage system component (gcvT), the maltose outer membrane porin/phage lambda receptor protein (lamB), and the fimbriae genes (fmABCDEFGHI). In addition, the native promoter of ppc was replaced with a stronger constitutive promoter. Strain 5491 also contains an inactivated arcA gene. Several native genes are overexpressed on a low-copy plasmid in both 5491 and 5717: ackA, pta, sucA, sucB and lpdA.

Table 52 shows OD and LCMS data for BDO, 4HB, acetate and pyruvate levels of the strain with the crr-variant ECKh-5491(2311A) with respect to the control, 5717 (crr-wt). Four replicates of each strain were cultured in 24-well plates. All concentrations are in mM and were measured after 24 hours of culture time. Higher BDO and 4HB were observed in cells with the truncated crr variant 2311A. Additionally, the crr variant produced lower levels of the byproducts ethanol and pyruvate. Gene sequence for 2311A (crr*) SEQ ID NO:187.

TABLE 52
BDO, 4HB, acetate and pyruvate levels of the
strain with the crr-variant ECKh-5491(2311A)
with respect to the control, 5717 (crr-wt).

| Host | OD 24 h | BDO | 4HB | Acetate | Pyruvate |
|---|---|---|---|---|---|
| 5491 | 19.82 | 216.0 | 22.8 | 4.8 | 1.1 |
| 5491 | 11.46 | 247.0 | 24.6 | 3.2 | 1.1 |
| 5491 | 12.67 | 248.0 | 27.6 | 4.3 | 1.1 |
| 5491 | 7.14 | 248.0 | 28.3 | 3.6 | 1.1 |
| 5717 | 37.81 | 199.0 | 18.1 | 64.9 | 46.3 |
| 5717 | 30.89 | 168.0 | 18.7 | 69.7 | 50.4 |
| 5717 | 22.97 | 179.0 | 18.9 | 78.3 | 52.4 |
| 5717 | 25.43 | 157.0 | 18.2 | 48.0 | 33.1 |

Gene sequence for 2311A (crr*):
atgggtttgttcgataaactgaaatctctggtttccgacgacaagaagga
taccggaactattgagatcattgctccgctctctggcgagatcgtcaata
tcgaagacgtgccggatgtcgtttttgcggaaaaaatcgttggtgatggt
attgctatcaaaccaacgggtaacaaaatggtcgcgccagtagacggcac
cattggtaaaatctttgaaaccaaccacgcattctctatcgaatctgata
gcggcgttgaactgttcgtccacttcggtatcgacaccgttgaactgaaa
ggcgaaggcttcaagcgtattgctgaagaaggtcagcgcgtgaaagttgg
cgatactgtcattgaatttgatctgccgctgctggaagagaaagccaagt
ctaccctgactccggttgttatctccaacatggacgaaatcaaagaactg
atcaaactgtccggtagcgtaaccgtgggtgaaacccataagcgctaa
(SEQ ID NO: 187)

### Example XXXXI

### Additional BDO and 4HB Producing Strains with Enzyme Variants

This examples describes enzyme variants providing production of BDO, 4HB and/or other intermediates in a 4HB or BDO pathway.

Variants of cat2 were generated using site-directed mutagenesis and/or saturation mutagenesis and/or error-prone PCR and/or directed evolution. Variants were screened for production of products and/or byproducts such as BDO or ethanol in high-throughput assays or in in vitro assays as well as for product inhibition, such as with BDO. Additional variants were generated by random combinations of mutations.

Exemplary screening methods for Cat2 activity include, for example, Cat2 in vitro activity assay and in vivo assay for BDO or BDO and ethanol production. With respect to the Cat2 in vitro activity, the respective clones carrying a plasmid with a Strep-tagged gene construct were grown in LB medium and induced with IPTG (0.2 mM) for Cat2 expression. Cells were collected by centrifugation and then lysed (either chemically or by sonication). Cell debris was removed by centrifugation and the obtained supernatant diluted in binding buffer (12.5 mM Tris/HCl pH 7.5, 1 mM EDTA, 70 mM NaCl). 200 μl of the diluted supernatant was added per well to a StrepTactin coated plate. The plate was incubated for 2 hours at 4° C. to allow adsorption of the strep-tagged protein to the plate. After the incubation, unbound lysate components were removed by washing three times with wash buffer (200 μl of 0.05% Tween 20 in phosphate buffered saline). Cat2 activity was measured by monitoring the production of acetate after addition of substrate (20 mM 4-hydroxybutyrate, 2 mM acetyl-CoA) by addition of a commercially available acetate quantification reagent. To measure inhibition of Cat2, the assay was performed in the absence and presence of BDO (up to 1M).

With respect to the in vivo assay for measuring BDO and ethanol, a high throughput in vivo assay was performed. Briefly, a BDO production strain harboring variants of the cat2 gene was grown in 96 well-plate format. After 16 hours of growth in a micro-aerobic environment, the culture supernatant was assayed for BDO or ethanol. Both detection methods utilized an enzymatic conversion of the alcohol (1,4 butanediol or ethanol) to the respective aldehyde (4-hydroxybutyraldehyde or acetaldehyde) via formation of NAD(P)H, monitored at 340 nm. The assays were conducted in 96 well polystyrene plates with 200 assay mixture. The assay mixture for BDO detection contained 50 mM Tris pH 7.4, 5 mM NADP+ and 3 μg of a BDO-specific alcohol dehydrogenase. The assay mixture for ethanol detection contained 50 mM Tris pH7.4, 5 mM NAD+ and 3 μg of an ethanol-specific alcohol dehydrogenase. The enzyme reaction was initiated with addition of 10 μl of spent media and allowed to proceed for 30 min at 42° C. The final concentration of BDO or ethanol is determined by extrapolation to a standard curve. Variant enzymes were identified that exhibited increased activity in the presence of BDO. Certain variants exhibited activity of about 1.2 fold, about 2.5-fold, or about 2.8-fold higher activity in the presence of BDO than wild type. Variants with combinations of two or more variant amino acid positions exhibited activities greater than wild type. Generating enzyme variants by combining active variant amino acid positions resulted in enzyme variants with improved properties.

Table 53 provides exemplary cat2 sequences based on homology. One skilled in the art will readily understand that such sequences can be analyzed with routine and well known methods for aligning sequences (for example BLAST, blast.ncbi.nlm.nih.gov; Altschul et al., “J. Mol. Biol. 215:403-410 (1990)). Such alignments can provide information on conserved residues that can be utilized to identify a consensus sequence for preserving enzyme activity as well as positions for generating further enzyme variants.

TABLE 53
Exemplary cat2 sequences.

| Description | Accession No. |
|---|---|
| hypothetical protein HMPREF0404_00865 | ZP_08581574.1 |
| [Fusobacterium sp. 21_1A] | GI:336400801 |
| >gb\|EGN64817.1\| hypothetical protein | (SEQ ID NO: 248) |
| HMPREF0404_00865 [Fusobacterium sp. | |
| 21_1A] | |
| acetyl-CoA hydrolase [Fusobacterium sp. | ZP_05814664.1 |
| 3_1_33]>gb\|EEW95213.1\| acetyl-CoA | GI:260494534 |
| hydrolase [Fusobacterium sp. 3_1_33] | (SEQ ID NO: 249) |
| 4-hydroxybutyrate coenzyme A | ZP_04575154.1 |
| transferase [Fusobacterium sp. 7_1] | GI:237744673 |
| >ref\|ZP_06523842.1\| 4-hydroxybutyrate | (SEQ ID NO: 250) |
| coenzyme A transferase [Fusobacterium sp. | |
| D11]>gb\|EEO42114.1\| 4-hydroxybutyrate | |
| coenzyme A transferase [Fusobacterium | |
| sp. 7_1]>gb\|EFD80031.1\| 4-hydroxybutyrate | |
| coenzyme A transferase | |
| [Fusobacterium sp. D11] | |
| 4-hydroxybutyrate CoA-transferase | ZP_08598403.1 |
| [Fusobacterium sp. 11_3_2] >gb\|EGN63628.1\| | GI:336418124 |
| 4-hydroxybutyrate CoA-transferase | (SEQ ID NO: 251) |
| [Fusobacterium sp. 11_3_2] | |
| acetyl-CoA hydrolase [Desulfomonile tiedjei | YP_006446580.1 |
| DSM 6799] >gb\|AFM24316.1\| acetyl- | GI:392409973 |
| CoA hydrolase [Desulfomonile tiedjei | (SEQ ID NO: 252 |
| DSM 6799] | |
| 4-hydroxybutyrate coenzyme A transferase | ZP_04054791.1 |
| [Porphyromonas uenonis 60-3] | GI:228469852 |
| >gb\|EEK17275.1\| 4-hydroxybutyrate | (SEQ ID NO: 253) |
| coenzyme A transferase [Porphyromonas | |
| uenonis 60-3] | |
| acetyl-CoA hydrolase/transferase | ZP_10325539.1 |
| [Pelosinus fermentans DSM 17108] | GI:392960066 |
| >gb\|EIW32362.1\| acetyl-CoA hydrolase/ | (SEQ ID NO: 254) |
| transferase [Pelosinus fermentans DSM | |
| 17108] | |
| 4-hydroxybutyrate coenzyme A transferase | ZP_08710943.1 |
| [Megasphaera sp. UPII 135-E] | GI:342218327 |
| >gb\|EGS33214.1\| 4-hydroxybutyrate | (SEQ ID NO: 255) |
| coenzyme A transferase | |
| [Megasphaera sp. UPI | |
| 135-E] | |
| acetyl-CoA hydrolase/transferase | ZP_15516677.1 |
| [Pelosinus fermentans B4] >ref\|ZP_15522476.1\| | GI:421053705 |
| acetyl-CoA hydrolase/transferase | (SEQ ID NO: 256) |
| [Pelosinus fermentans B3] >ref\|ZP_15526118.1\| | |
| acetyl-CoA hydrolase/transferase [Pelosinus | |
| fermentans A12] >ref\|ZP_15532060.1\| | |
| acetyl-CoA hydrolase/transferase | |
| [Pelosinus fermentans A11] >gb\|EIW19212.1\| | |
| acetyl-CoA hydrolase/transferase | |
| [Pelosinus fermentans B4] >gb\|EIW25056.1\| acetyl- | |
| CoA hydrolase/transferase [Pelosinus | |
| fermentans A11] >gb\|EIW34652.1\| acetyl-CoA | |
| hydrolase/transferase [Pelosinus fermentans | |
| B3] >gb\|EIW37648.1\| acetyl-CoA | |
| hydrolase/transferase [Pelosinus | |
| fermentans A12] | |
| 4-hydroxybutyrate CoA-transferase | ZP_06027623.1 |
| [Fusobacterium periodonticum ATCC 33693] | GI:262068011 |
| >gb\|EFE85797.1\| 4-hydroxybutyrate CoA- | (SEQ ID NO: 257) |
| transferase [Fusobacterium periodonticum | |
| ATCC 33693] | |
| 4-hydroxybutyrate CoA-transferase | ZP_16968033.1 |
| [Fusobacterium nucleatum subsp. animalis ATCC | GI:422945708 |
| 51191]>gb\|EGQ78661.1\| 4-hydroxybutyrate | (SEQ ID NO: 258) |
| CoA-transferase [Fusobacterium | |
| nucleatum subsp. animalis ATCC 51191] | |
| 4-hydroxybutyrate coenzyme A | ZP_04572671.1 |
| transferase [Fusobacterium sp. 4_1_13] | GI:237742190 |
| >gb\|EEO40050.1\| 4-hydroxybutyrate | (SEQ ID NO: 259) |
| coenzyme A transferase [Fusobacterium sp. | |
| 4_1_13] | |
| 4-hydroxybutyrate coenzyme A | ZP_08689706.1 |
| transferase [Fusobacterium sp. 2_1_31] | GI:340752912 |
| >ref\|ZP_16398352.1\| hypothetical | (SEQ ID NO: 260) |
| protein FPOG_01423 [Fusobacterium | |
| periodonticum D10] >gb\|EEO37750.1\| | |
| 4-hydroxybutyrate coenzyme A transferase | |
| [Fusobacterium sp. 2_1_31]>gb\|EKA92776.1\| | |
| hypothetical protein FPOG_01423 | |
| [Fusobacterium periodonticum D10] | |
| 4-hydroxybutyrate CoA-transferase | ZP_15973607.1 |
| [Fusobacterium nucleatum ChDC F128] | GI:421527002 |
| >gb\|EJU07208.1\| 4-hydroxybutyrate | (SEQ ID NO: 261) |
| CoA-transferase [Fusobacterium nucleatum | |
| ChDC F128] | |
| 4-hydroxybutyrate CoA-transferase | ZP_06747486.1 |
| [Fusobacterium sp. 1_1_41FAA] | GI:294782160 |
| >gb\|EFG28576.1\| 4-hydroxybutyrate | (SEQ ID NO: 262) |
| CoA-transferase [Fusobacterium sp. | |
| 1_1_41FAA] | |
| 4-hydroxybutyrate CoA-transferase | ZP_15604676.1 |
| [Fusobacterium nucleatum subsp. fusiforme | GI:421144770 |
| ATCC 51190]>gb\|EJG09667.1\| | (SEQ ID NO: 263) |
| 4-hydroxybutyrate CoA-transferase [Fusobacterium | |
| nucleatum subsp. fusiforme ATCC 51190] | |
| hypothetical protein HMPREF9942_00351 | ZP_17124213.1 |
| [Fusobacterium nucleatum subsp. | GI:423136570 |
| animalis F0419]>gb\|EHO79348.1\| | (SEQ ID NO: 264) |
| hypothetical protein HMPREF9942_00351 | |
| [Fusobacterium nucleatum subsp. animalis F0419] | |
| acetyl-CoA hydrolase [Fusobacterium | ZP_04971624.1 |
| nucleatum subsp. polymorphum ATCC 10953] | GI:254304266 |
| >gb\|EDK89708.1\| acetyl-CoA hydrolase | (SEQ ID NO: 265) |
| [Fusobacterium nucleatum subsp. | |
| polymorphum ATCC 10953] | |
| acetyl-CoA hydrolase/transferase [Roseiflexus | YP_001275270.1 |
| sp. RS-1] >gb\|ABQ89320.1\| acetyl- | GI:148655065 |
| CoA hydrolase/transferase [Roseiflexus sp. RS-1] | (SEQ ID NO: 266) |
| acetyl-CoA hydrolase/transferase | YP_004441567.1 |
| [Porphyromonas asaccharolytica DSM 20707] | GI:332299646 |
| >gb\|AEE12399.1\| acetyl-CoA hydrolase/ | (SEQ ID NO: 267) |
| transferase [Porphyromonas asaccharolytica | |
| DSM 20707] | |
| 4-hydroxybutyrate CoA-transferase | EMP15468.1 |
| [Fusobacterium nucleatum CC53] | GI:465143275 |
| | (SEQ ID NO: 268) |
| butyryl:4-hydroxybutyrate CoA | YP_002138233.1 |
| transferase [Geobacter bemidjiensis Bem] | GI:197117806 |
| >gb\|ACH38437.1\| butyryl:4-hydroxybutyrate | (SEQ ID NO: 269) |
| coenzyme A transferase [Geobacter | |
| bemidjiensis Bem] | |
| acetyl-CoA hydrolase [Fusobacterium | ZP_05550921.1 |
| sp. 3_1_36A2] >gb\|EEU32577.1\| acetyl-CoA | GI:256845463 |
| hydrolase [Fusobacterium sp. 3_1_36A2] | (SEQ ID NO: 270) |
| 4-hydroxybutyrate CoA-transferase | ZP_06750521.1 |
| [Fusobacterium sp. 3_1_27]>gb\|EFG34309.1\| 4- | GI:294785233 |
| hydroxybutyrate CoA-transferase | (SEQ ID NO: 271) |
| [Fusobacterium sp. 3_1_27] | |
| 4-hydroxybutyrate CoA-transferase | ZP_07455129.1 |
| [Eubacterium yurii subsp. margaretiae ATCC | GI:306821529 |
| 43715]>gb\|EFM38414.1\| 4-hydroxybutyrate | (SEQ ID NO: 272) |
| CoA-transferase [Eubacterium yurii | |
| subsp. margaretiae ATCC 43715] | |
| acetyl-CoA hydrolase/transferase | YP_001433830.1 |
| [Roseiflexus castenholzii DSM 13941] | GI:156743701 |
| >gb\|ABU59812.1\| acetyl-CoA hydrolase/ | (SEQ ID NO: 273) |
| transferase [Roseiflexus castenholzii DSM | |
| 13941] | |
| 4-hydroxybutyrate coenzyme A transferase | NP_603518.1 |
| [Fusobacterium nucleatum subsp. | GI:19703956 |
| nucleatum ATCC 25586] >ref\|ZP_06870070.1\| | (SEQ ID NO: 274) |
| 4-hydroxybutyrate CoA-transferase | |
| [Fusobacterium nucleatum subsp. | |
| nucleatum ATCC 23726] >gb\|AAL94817.1\| 4- | |
| hydroxybutyrate coenzyme A transferase | |
| [Fusobacterium nucleatum subsp. nucleatum | |
| ATCC 25586]>gb\|EFG96121.1\| | |
| 4-hydroxybutyrate CoA-transferase [Fusobacterium | |
| nucleatum subsp. nucleatum ATCC 23726] | |
| 4-hydroxybutyrate coenzyme A transferase | ZP_07821110.1 |
| [Porphyromonas asaccharolytica | GI:313887421 |
| PR426713P-I]>gb\|EFR33883.1\| | (SEQ ID NO: 275) |
| 4-hydroxybutyrate coenzyme A transferase | |
| [Porphyromonas asaccharolytica PR426713P-I] | |
| 4-hydroxybutyrate CoA-transferase | ZP_09999308.1 |
| [Imtechella halotolerans K1] >gb\|EID75748.1\| 4- | GI:384098189 |
| hydroxybutyrate CoA-transferase | (SEQ ID NO: 276) |
| [Imtechella halotolerans K1] | |
| 4-hydroxybutyrate CoA-transferase | ZP_16420483.1 |
| [Fusobacterium nucleatum subsp. polymorphum | GI:422339525 |
| F0401]>gb\|EHG18330.1\| 4-hydroxybutyrate | (SEQ ID NO: 277) |
| CoA-transferase [Fusobacterium | |
| nucleatum subsp. polymorphum F0401] | |
| 4-hydroxybutyrate coenzyme A transferase | ZP_10829831.1 |
| [Eubacterium sp. AS15] >gb\|EJP19825.1\| | GI:402310872 |
| 4-hydroxybutyrate coenzyme A | (SEQ ID NO: 278) |
| transferase [Eubacterium sp. AS15] | |
| acetyl-CoA hydrolase [Desulfosporosinus | YP_006466984.1 |
| acidiphilus SJ4] >gb\|AFM41652.1\| acetyl- | GI:392425990 |
| CoA hydrolase [Desulfosporosinus | (SEQ ID NO: 279) |
| acidiphilus SJ4] | |
| putative butyryl-CoA:acetate CoA-transferase | ZP_09385293.1 |
| [Flavonifractor plautii ATCC 29863] | GI:365844447 |
| >gb\|EHM42532.1\| putative butyryl-CoA: | (SEQ ID NO: 280) |
| acetate CoA-transferase [Flavonifractor | |
| plautii ATCC 29863] | |
| hypothetical protein CarbS_08493 | ZP_10774427.1 |
| [Clostridium arbusti SL206] | GI:399888550 |
| | (SEQ ID NO: 281) |
| acetyl-CoA hydrolase/transferase | ZP_05395303.1 |
| [Clostridium carboxidivorans P7] | GI:255528520 |
| >ref\|ZP_06857150.1\| acetyl-CoA hydrolase/ | (SEQ ID NO: 282) |
| transferase [Clostridium carboxidivorans | |
| P7] >gb\|EET84247.1\| acetyl-CoA hydrolase/ | |
| transferase [Clostridium carboxidivorans | |
| P7] >gb\|EFG86097.1\| acetyl-CoA hydrolase/ | |
| transferase [Clostridium carboxidivorans | |
| P7] | |
| 4-hydroxybutyrate CoA-transferase | YP_005014371.1 |
| [Tannerella forsythia ATCC 43037] | GI:375255204 |
| >gb\|AEW22101.1\| 4-hydroxybutyrate | (SEQ ID NO: 283) |
| coenzyme A transferase [Tannerella forsythia | |
| ATCC 43037] | |
| acetyl-CoA hydrolase/transferase | ZP_07932276.1 |
| domain-containing protein [Anaerostipes sp. | GI:317472972 |
| 3_2_56FAA]>gb\|EFV21594.1\| acetyl-CoA | (SEQ ID NO: 284) |
| hydrolase/transferase domain-containing | |
| protein [Anaerostipes sp. 3_2_56FAA] | |
| 4-hydroxybutyrate:acetyl-CoA transferase | ZP_07924174.1 |
| [Fusobacterium sp. 3_1_5R] | GI:317059689 |
| >gb\|EFS22200.1\| 4-hydroxybutyrate: | (SEQ ID NO: 285) |
| acetyl-CoA transferase [Fusobacterium sp. | |
| 3_1_5R] | |
| acetyl-CoA hydrolase/transferase [Geobacter | YP_003022652.1 |
| sp. M21]>gb\|ACT18894.1\| acetyl-CoA | GI:253701463 |
| hydrolase/transferase [Geobacter sp. M21] | (SEQ ID NO: 286) |
| acetyl-CoA hydrolase [Desulfosporosinus | YP_006466004.1 |
| acidiphilus SJ4] >gb\|AFM40672.1\| acetyl- | GI:392425010 |
| CoA hydrolase [Desulfosporosinus | (SEQ ID NO: 287) |
| acidiphilus SJ4] | |
| hypothetical protein CKL_3018 | YP_001396397.1 |
| [Clostridium kluyveri DSM 555] | GI:153955632 |
| >ref\|YP_002473131.1\| hypothetical protein | (SEQ ID NO: 288) |
| CKR_2666 [Clostridium kluyveri NBRC | |
| 12016]>sp\|P38942.3\|CAT2_CLOK5 | |
| RecName: Full = 4-hydroxybutyrate coenzyme | |
| A transferase >gb\|EDK35026.1\| Cat2 | |
| [Clostridium kluyveri DSM 555] | |
| >dbj\|BAH07717.1\| hypothetical protein | |
| [Clostridium kluyveri NBRC 12016] | |
| LOW QUALITY PROTEIN: acetyl-CoA | ZP_10305985.1 |
| hydrolase [Thermoanaerobacter | GI:392940341 |
| siderophilus SR4]>gb\|EIW00535.1\| | (SEQ ID NO: 289) |
| LOW QUALITY PROTEIN: acetyl-CoA | |
| hydrolase [Thermoanaerobacter | |
| siderophilus SR4] | |
| 4-hydroxybutyrate:acetyl-CoA transferase | ZP_07914775.1 |
| [Fusobacterium gonidiaformans ATCC | GI:315918535 |
| 25563] >ref\|ZP_07924054.1\| | (SEQ ID NO: 290) |
| 4-hydroxybutyrate:acetyl-CoA transferase | |
| [Fusobacterium sp. 3_1_5R] | |
| >gb\|EFS22080.1\| 4-hydroxybutyrate:acetyl-CoA | |
| transferase [Fusobacterium sp. 3_1_5R] | |
| >gb\|EFS29245.1\| 4-hydroxybutyrate:acetyl- | |
| CoA transferase [Fusobacterium | |
| gonidiaformans ATCC 25563] | |
| 4-hydroxybutyrate coenzyme A transferase | ZP_08693772.1 |
| [Fusobacterium varium ATCC 27725] | GI:340757169 |
| >gb\|EES63001.1\| 4-hydroxybutyrate | (SEQ ID NO: 291) |
| coenzyme A transferase [Fusobacterium varium | |
| ATCC 27725] | |
| 4-hydroxybutyrate coenzyme A transferase | ZP_14365187.1 |
| [Fusobacterium necrophorum subsp. | GI:419841825 |
| funduliforme ATCC 51357] >gb\|EIJ68663.1\| | (SEQ ID NO: 292) |
| 4-hydroxybutyrate coenzyme A | |
| transferase [Fusobacterium necrophorum | |
| subsp. funduliforme ATCC 51357] | |
| hypothetical protein ANACAC_00165 | ZP_02417601.1 |
| [Anaerostipes caccae DSM 14662] | GI: 167745474 |
| >gb\|EDR99324.1\| 4-hydroxybutyrate | (SEQ ID NO: 293) |
| coenzyme A transferase [Anaerostipes caccae | |
| DSM 14662] | |
| 4-hydroxybutyrate coenzyme A | ZP_14664473.1 |
| transferase [Clostridium sp. MSTE9] | GI:420157643 |
| >gb\|EJF39044.1\| 4-hydroxybutyrate | (SEQ ID NO: 294) |
| coenzyme A transferase [Clostridium sp. | |
| MSTE9] | |
| hypothetical protein ANASTE_01215 | ZP_02862002.1 |
| [Anaerofustis stercorihominis DSM 17244] | GI: 169334809 |
| >gb\|EDS71513.1\| 4-hydroxybutyrate | (SEQ ID NO: 295) |
| coenzyme A transferase [Anaerofustis | |
| stercorihominis DSM 17244] | |
| 4-hydroxybutyrate CoA-transferase | ABA39275.1 |
| [Anaerostipes caccae] | GI:76096774 |
| | (SEQ ID NO: 296) |
| 4-Hydroxybutyrate CoA-transferase | CAB60036 |
| [Clostridium aminobutyricum]. | GI:188032706 |
| | (SEQ ID NO: 297) |
| 4-Hydroxybutyrate CoA-transferase | CAB60036.2 |
| [Clostridium aminobutyricum] | GI:188032706 |
| | (SEQ ID NO: 298) |
| Chain A, Crystal Structure Of | 3GK7 A |
| 4-Hydroxybutyrate | GI:281500759 |
| Coa-Transferase From Clostridium | (SEQ ID NO: 299) |
| Aminobutyricum >pdb\|3GK7\|B Chain B, | |
| Crystal Structure Of 4-Hydroxybutyrate | |
| Coa-Transferase From Clostridium | |
| Aminobutyricum >pdb\|3QDQ\|A Chain A, | |
| Complex Between 4-Hydroxybutyrate | |
| Coa-Transferase From Clostridium | |
| Aminobutyricum And Coa | |
| 4-hydroxybutyrate CoA-transferase | ZP_05427217.1 |
| [Eubacterium saphenum ATCC 49989] | GI:255994082 |
| >gb\|EEU03839.1\| 4-hydroxybutyrate | (SEQ ID NO: 300) |
| CoA-transferase [Eubacterium saphenum | |
| ATCC 49989] | |
| putative butyryl-CoA:acetate CoA-transferase | ZP_17072579.1 |
| [Clostridium difficile 002-P50-2011] | GI:423084074 |
| >ref\|ZP_17075260.1\| putative butyryl- | (SEQ ID NO: 301) |
| CoA:acetate CoA-transferase [Clostridium | |
| difficile 050-P50-2011]>gb\|EHJ25154.1\| | |
| putative butyryl-CoA:acetate CoA- | |
| transferase [Clostridium difficile | |
| 002-P50-2011] >gb\|EHJ27941.1\| putative butyryl- | |
| CoA:acetate CoA-transferase | |
| [Clostridium difficile 050-P50-2011] | |
| 4-hydroxybutyrate CoA-transferase | YP_001088854.1 |
| [Clostridium difficile 630] >ref\|ZP_05272394.1\| | GI:126699957 |
| 4-hydroxybutyrate CoA transferase | (SEQ ID NO: 302) |
| [Clostridium difficile QCD-66c26] | |
| >ref\|ZP_05322787.1\| 4-hydroxybutyrate | |
| CoA transferase [Clostridium difficile CIP | |
| 107932] >ref\|ZP_05330465.1\| | |
| 4-hydroxybutyrate CoA transferase [Clostridium | |
| difficile QCD-63q42]>ref\|ZP_05356638.1\| | |
| 4-hydroxybutyrate CoA transferase | |
| [Clostridium difficile QCD-76w55] | |
| >ref\|ZP_05385401.1\| 4-hydroxybutyrate CoA | |
| transferase [Clostridium difficile | |
| QCD-97634] >ref\|ZP_05397740.1\| 4- | |
| hydroxybutyrate CoA transferase | |
| [Clostridium difficile QCD-37x79] | |
| >ref\|YP_003215203.1\| 4-hydroxybutyrate | |
| CoA transferase [Clostridium difficile | |
| CD196]>ref\|YP_003218712.1\| | |
| 4-hydroxybutyrate CoA transferase [Clostridium | |
| difficile R20291] >ref\|ZP_07407078.1\| | |
| 4-hydroxybutyrate CoA transferase | |
| [Clostridium difficile QCD-32g58] | |
| >ref\|YP_006199406.1\| 4-hydroxybutyrate CoA | |
| transferase [Clostridium difficile BI1] | |
| >ref\|ZP_17080166.1\| putative butyryl- | |
| CoA:acetate CoA-transferase [Clostridium | |
| difficile 70-100-2010] >emb\|CAJ69226.1\| | |
| 4-hydroxybutyrate CoA transferase | |
| [Clostridium difficile 630] >emb\|CBA64187.1\| 4- | |
| hydroxybutyrate CoA transferase | |
| [Clostridium difficile CD196] >emb\|CBE05384.1\| | |
| 4-hydroxybutyrate CoA transferase | |
| [Clostridium difficile R20291] >gb\|EHJ35600.1\| | |
| putative butyryl-CoA:acetate CoA- | |
| transferase [Clostridium difficile 70-100-2010] | |
| 4-hydroxybutyrate CoA transferase | ZP_05351533.1 |
| [Clostridium difficile ATCC 43255] | GI:255307362 |
| | (SEQ ID NO: 303) |
| 4-hydroxybutyrate CoA transferase | ZP_05401721.1 |
| [Clostridium difficile QCD-23m63] | GI:255656312 |
| >ref\|ZP_06892006.1\| 4-hydroxybutyrate | (SEQ ID NO: 304) |
| CoA-transferase [Clostridium difficile | |
| NAP08]>ref\|ZP_06902665.1\| | |
| 4-hydroxybutyrate CoA-transferase [Clostridium | |
| difficile NAP07] >gb\|EFH07842.1\| | |
| 4-hydroxybutyrate CoA-transferase [Clostridium | |
| difficile NAP08] >gb\|EFH16309.1\| | |
| 4-hydroxybutyrate CoA-transferase [Clostridium | |
| difficile NAP07] | |
| 4-hydroxybutyrate coenzyme A | ZP_14663205.1 |
| transferase [Clostridium sp. MSTE9] | GI:420156362 |
| >gb\|EJF40677.1\| 4-hydroxybutyrate | (SEQ ID NO: 305) |
| coenzyme A transferase [Clostridium sp. | |
| MSTE9] | |
| hypothetical protein CarbS_12961 | ZP_10775305.1 |
| [Clostridium arbusti SL206] | GI:399889428 |
| | (SEQ ID NO: 306) |
| hypothetical protein HMPREF0995_05302 | ZP_09534466.1 |
| [Lachnospiraceae bacterium | GI:373120404 |
| 7_1_58FAA]>gb\|EHO23268.1\| | (SEQ ID NO: 307) |
| hypothetical protein HMPREF0995 05302 | |
| [Lachnospiraceae bacterium 7_1_58FAA] | |
| hypothetical protein HMPREF0995_04657 | ZP_09533821.1 |
| [Lachnospiraceae bacterium | GI:373119730 |
| 7_1_58FAA]>gb\|EHO27239.1\| | (SEQ ID NO: 308) |
| hypothetical protein HMPREF0995_04657 | |
| [Lachnospiraceae bacterium 7_1_58FAA] | |
| acetyl-CoA hydrolase/transferase | YP_001309226.1 |
| [Clostridium beijerinckii NCIMB 8052] | GI:150016972 |
| >gb\|ABR34270.1\| acetyl-CoA hydrolase/ | (SEQ ID NO: 309) |
| transferase [Clostridium beijerinckii NCIMB | |
| 8052] | |
| 4-hydroxybutyrate coenzyme A | AAA92344.1 |
| transferase [Clostridium kluyveri DSM 555] | GI:1228101 |
| | (SEQ ID NO: 310) |
| acetyl-CoA hydrolase/transferase | YP_004253244.1 |
| [Odoribacter splanchnicus DSM 20712] | GI:325280702 |
| >gb\|ADY33064.1\| acetyl-CoA hydrolase/ | (SEQ ID NO: 311) |
| transferase [Odoribacter splanchnicus DSM | |
| 20712] | |
| 4-hydroxybutyrate CoA-transferase | YP_001928841.1 |
| [Porphyromonas gingivalis ATCC 33277] | GI: 188994589 |
| >dbj\|BAG33244.1\| 4-hydroxybutyrate | (SEQ ID NO: 312) |
| CoA-transferase [Porphyromonas gingivalis | |
| ATCC 33277] | |
| hypothetical protein FUAG_02467 | ZP_10973595.1 |
| [Fusobacterium ulcerans ATCC 49185] | GI:404368238 |
| >gb\|EFS26952.1\| hypothetical protein | (SEQ ID NO: 313) |
| FUAG_02467 [Fusobacterium ulcerans ATCC | |
| 49185] | |
| 4-hydroxybutyrate CoA-transferase | ZP_08598358.1 |
| [Fusobacterium sp. 11_3_2] >gb\|EGN63583.1\| 4- | GI:336418079 |
| hydroxybutyrate CoA-transferase | (SEQ ID NO: 314) |
| [Fusobacterium sp. 11_3_2] | |
| 4-hydroxybutyrate CoA-transferase | EMP16460.1 |
| [Fusobacterium nucleatum CC53] | GI:465144883 |
| | (SEQ ID NO: 315) |
| 4-hydroxybutyrate:acetyl-CoA CoA | ZP_00144049.1 |
| transferase [Fusobacterium nucleatum subsp. | GI:34763077 |
| vincentii ATCC 49256] >gb\|EAA24344.1\| | (SEQ ID NO: 316) |
| 4-hydroxybutyrate:acetyl-CoA CoA | |
| transferase [Fusobacterium nucleatum | |
| subsp. vincentii ATCC 49256] | |
| 4-hydroxybutyrate coenzyme A | ZP_08691841.1 |
| transferase [Fusobacterium sp. D12] | GI:340755139 |
| >ref\|ZP_09528784.1\| hypothetical | (SEQ ID NO: 317) |
| protein HMPREF9466_02817 [Fusobacterium | |
| necrophorum subsp. funduliforme 1_1_36S] | |
| >ref\|ZP_15946533.1\| 4-hydroxybutyrate | |
| coenzyme A transferase [Fusobacterium | |
| necrophorum subsp. funduliforme Fnf 1007] | |
| >gb\|EFS22986.1\| 4-hydroxybutyrate | |
| coenzyme A transferase [Fusobacterium sp. | |
| D12]>gb\|EHO16876.1\| hypothetical protein | |
| HMPREF9466_02817 [Fusobacterium | |
| necrophorum subsp. funduliforme 1_1_36S] | |
| >gb\|EJU18967.1\| 4-hydroxybutyrate | |
| coenzyme A transferase [Fusobacterium | |
| necrophorum subsp. funduliforme Fnf 1007] | |
| TDC60]>dbj\|BAK25964.1\| 4-hydroxybutyrate | YP_004510530.1 |
| CoA-transferase [Porphyromonas | GI:334147601 |
| gingivalis TDC60] | (SEQ ID NO: 318) |
| 4-hydroxybutyrate CoA-transferase | NP_904965.1 |
| [Porphyromonas gingivalis W83] | |
| >ref\|ZP_14485385.1\| 4-hydroxybutyrate s | GI:34540486 |
| coenzyme A transferase [Porphyromona | (SEQ ID NO: 319) |
| gingivalis W50] >gb\|AAQ65864.1\| | |
| 4-hydroxybutyrate CoA-transferase | |
| [Porphyromonas gingivalis W83] >gb\|EIW94739.1\| | |
| 4-hydroxybutyrate coenzyme A | |
| transferase [Porphyromonas gingivalis W50] | |
| hypothetical protein CKL_3595 | YP_001396957.1 |
| [Clostridium kluyveri DSM 555] | GI:153956192 |
| >ref\|YP_002473639.1\| hypothetical protein | (SEQ ID NO: 320) |
| CKR_3174 [Clostridium kluyveri NBRC | |
| 12016]>gb\|EDK35586.1\| Cat3 [Clostridium | |
| kluyveri DSM 555] >dbj\|BAH08225.1\| | |
| hypothetical protein [Clostridium kluyveri | |
| NBRC 12016] | |
| hypothetical protein HMPREF0402_00217 | ZP_09586344.1 |
| [Fusobacterium sp. 12_1B] | GI:373495791 |
| >gb\|EHO84398.1\| hypothetical protein | (SEQ ID NO: 321) |
| HMPREF0402_00217 [Fusobacterium sp. | |
| 12_1B] | |
| 4-hydroxybutyrate:acetyl-CoA transferase | ZP_04572117.1 |
| [Fusobacterium sp. 4_1_13] | GI:237741636 |
| >ref\|ZP_06751042.1\| 4-hydroxybutyrate | (SEQ ID NO: 322) |
| CoA-transferase [Fusobacterium sp. 3_1_27] | |
| >gb\|EEO39496.1\| 4-hydroxybutyrate: | |
| acetyl-CoA transferase [Fusobacterium sp. | |
| 4_1_13]>gb\|EFG34830.1\|4-hydroxybutyrate | |
| CoA-transferase [Fusobacterium sp. | |
| 3_1_27] | |
| 4-hydroxybutyrate CoA-transferase | NP_781174.1 |
| [Clostridium tetani E88] >gb\|AAO35111.1\| 4- | GI:28210230 |
| hydroxybutyrate coenzyme A | (SEQ ID NO: 323) |
| transferase [Clostridium tetani E88] | |
| 4-hydroxybutyrate:acetyl-CoA transferase | ZP_05550423.1 |
| [Fusobacterium sp. 3_1_36A2] | GI:256844965 |
| >gb\|EEU32079.1\| 4-hydroxybutyrate: | (SEQ ID NO: 324) |
| acetyl-CoA transferase [Fusobacterium sp. | |
| 3_1_36A2] | |
| 4-hydroxybutyrate CoA-transferase | YP_005055048.1 |
| [Filifactor alocis ATCC 35896]>gb\|EFE28990.1\| | GI:374308617 |
| 4-hydroxybutyrate CoA-transferase | (SEQ ID NO: 325) |
| [Filifactor alocis ATCC 35896] | |
| 4-hydroxybutyrate CoA-transferase | ZP_06983177.1 |
| [Bacteroidetes oral taxon 274 str. F0058] | GI:298373187 |
| >gb\|EFI17642.1\| 4-hydroxybutyrate CoA- | (SEQ ID NO: 326) |
| transferase [Bacteroidetes oral taxon 274 str. | |
| F0058] | |
| 4-hydroxybutyrate coenzyme A transferase | ZP_09046738.1 |
| [Clostridium sp. 7_3_54FAA] | GI:355622639 |
| >gb\|EHF07225.1\| 4-hydroxybutyrate | (SEQ ID NO: 327) |
| coenzyme A transferase [Clostridium sp. | |
| 7_3_54FAA] | |
| 4-hydroxybutyrate coenzyme A transferase | ZP_04389695.1 |
| [Porphyromonas endodontalis ATCC | GI:229495971 |
| 35406]>gb\|EEN82972.1\| 4-hydroxybutyrate | (SEQ ID NO: 328) |
| coenzyme A transferase | |
| [Porphyromonas endodontalis ATCC 35406] | |
| acetyl-CoA hydrolase/transferase | YP_004765391.1 |
| [Megasphaera elsdenii DSM 20460] | GI:348025587 |
| >emb\|CCC72564.1\| acetyl-CoA hydrolase/ | (SEQ ID NO: 329) |
| transferase [Megasphaera elsdenii DSM | |
| 20460] | |
| acetyl-CoA hydrolase/transferase | YP_002430388.1 |
| [Desulfatibacillum alkenivorans AK-01] | GI:218779070 |
| >gb\|ACL02920.1\| acetyl-CoA hydrolase/ | (SEQ ID NO: 330) |
| transferase [Desulfatibacillum alkenivorans | |
| AK-01] | |
| 4-hydroxybutyrate CoA-transferase | ZP_08935544.1 |
| [Peptoniphilus indolicus ATCC 29427] | GI:350566925 |
| >gb\|EGY77354.1\| 4-hydroxybutyrate | (SEQ ID NO: 331) |
| CoA-transferase [Peptoniphilus indolicus | |
| ATCC 29427] | |
| acetyl-CoA hydrolase/transferase | ZP_08976459.1 |
| [Desulfitobacterium metallireducens DSM 15288] | GI:354557200 |
| >gb\|EHC20214.1\| acetyl-CoA hydrolase/ | (SEQ ID NO: 332) |
| transferase [Desulfitobacterium | |
| metallireducens DSM 15288] | |
| acetyl-CoA hydrolase/transferase | ZP_08623452.1 |
| [Acetonema longum DSM 6540] >gb\|EGO65184.1\| | GI:338811223 |
| acetyl-CoA hydrolase/transferase | (SEQ ID NO: 333) |
| [Acetonema longum DSM 6540] | |
| Chain A, The Structure Of A Putative | 3EH7 A |
| 4-Hydroxybutyrate Coa-Transferase From | GI:217035437 |
| Porphyromonas Gingivalis W83 | (SEQ ID NO: 334) |
| acetyl-CoA hydrolase/transferase | ZP_08089122.1 |
| [Clostridium symbiosum WAL-14163] | GI:323483742 |
| >gb\|EGA95251.1\| acetyl-CoA hydrolase/ | (SEQ ID NO: 335) |
| transferase [Clostridium symbiosum WAL- | |
| 14163] | |
| acetyl-CoA hydrolase/transferase | ZP_06559383.1 |
| [Megasphaera genomosp. type_1 str. 28L] | GI:290967830 |
| >ref\|ZP_08542966.1\| putative butyryl-CoA: | (SEQ ID NO: 336) |
| acetate CoA-transferase [Megasphaera sp. | |
| UPII 199-6] >gb\|EFD94764.1\| acetyl-CoA | |
| hydrolase/transferase [Megasphaera | |
| genomosp. type_1 str. 28L]>gb\|EGL39418.1\| | |
| putative butyryl-CoA:acetate CoA- | |
| transferase [Megasphaera sp. UPII 199-6] | |
| acetyl-CoA hydrolase/transferase | YP_003702013.1 |
| [Syntrophothermus lipocalidus DSM 12680] | GI:297616854 |
| >gb\|ADI01448.1\| acetyl-CoA hydrolase/ | (SEQ ID NO: 337) |
| transferase [Syntrophothermus lipocalidus | |
| DSM 12680] | |
| acetyl-CoA hydrolase/transferase | ZP_08106938.1 |
| [Clostridium symbiosum WAL-14673] | GI:323692709 |
| >gb\|EGB19096.1\| acetyl-CoA hydrolase/ | (SEQ ID NO: 338) |
| transferase [Clostridium symbiosum WAL- | |
| 14673] | |

Table 54 provides exemplary cat2 enzyme variants. Table 54 shows exemplary enzyme variant positions with the corresponding amino acid for a particular position that were identified as providing a desirable activity, including cat2 activity, BDO production, and the like. It is understood that the individual cat2 variants such as those described in Table 54 can be used alone, or can be combined with any other variant amino acid position, as disclosed herein, including 2, 3, 4, 5, 6, 7, 8, 9, 10, greater than 10 and up to all variant amino acid positions to generate additional variants having desirable activities.

TABLE 54
Exemplary cat2 enzyme variants.

| | | Corresponding Positions | Exemplary Enzyme Sequence Indicating |
|---|---|---|---|
| Wild-Type Designation | Wild-Type Sequence | and Amino Acid Changes | Corresponding Positions |
| 4-hydroxybutyrate CoA- | 1 MQWQELYRQR VCSADEAVVD SLKPGTKVVF | 30L, 32G, 34A, 58Q, 108Y, | 1 MQWQELYRQR VCSADEAVVD SLKPGTKVVL |
| transferase | GHAAAAPVRF SQAMYRQREK LENITVFHML | 232R, 312S, 314V, 332Y | GGAAAAPVRF SQAMYRQREK LENITVFQML |
| [Porphyromonas | 61 YFGDAPHLAP EMRSHVHPTL NFLEGNSRPA | | 61 YFGDAPHLAP EMRSHVHPTL |
| gingivalis W83] | SRDRRVDFIP CHFHEVPELF RQGFFPLDVA | | NFLEGNSRPA SRDRRVDFIP CHFHEVPYLF |
| VERSION: NP_906037.1 | 121 VVQVSTPNEE GYCSFGVSCD YTKAAAECAP | | RQGFFPLDVA |
| GI:34541558 | VVVAEVNKQM PFIGGENLIH ISKLTHIIEV | | 121 VVQVSTPNEE GYCSFGVSCD |
| | 181 DEPIAEVLPP AISDLELRIG QNCASLIKDG | | YTKAAAECAP VVVAEVNKQM PFIGGENLIH |
| | DTLQLGIGGI PDAVLRALEG HKDLGIHTEM | | ISKLTHIIEV |
| | 241 FTDGVMRMIR KGIINGKKKT LHPEKVVTSL | | 181 DEPIAEVLPP AISDLELRIG QNCASLIKDG |
| | IFGSKELYDF VNNNPVIECY PVDYINNPDV | | DTLQLGIGGI PDAVLRALEG HRDLGIHTEM |
| | 301 IGKNDRMVSI NSCLEMDLMG QAASESIGYE | | 241 FTDGVMRMIR KGIINGKKKT |
| | QFSGSGGQVD FLRGAKRSKG GISIMAFPST | | LHPEKVVTSL IFGSKELYDF VNNNPVIECY |
| | 361 AKKGTESRIV PILKEGACVT TGRNEVDYVV | | PVDYINNPDV |
| | TEYGVARLRG ATLRQRAEAL TAIAHPDFRP | | 301 IGKNDRMVSI NSCVEMDLMG |
| | 421 ALEEEIRRRF E (SEQ ID NO: 188) | | QAASESIGYE QYSGSGGQVD FLRGAKRSKG |
| | | | GISIMAFPST |
| | | | 361 AKKGTESRIV PILKEGACVT |
| | | | TGRNEVDYVV TEYGVARLRG ATLRQRAEAL |
| | | | TAIAHPDFRP |
| | | | 421 ALEEEIRRRF E (SEQ ID NO: 199) |
| acetyl-CoA | 1 MPRSLTAGRR EMTAEEAVEL IESNHRVYIG | 29L, 31G, 33A, 57Q, 107Y, | 1 MPRSLTAGRR EMTAEEAVEL |
| hydrolase/transferase | GGCGVPIPLL DALVARAPEL RNVEIIHMLT | 231R, 311S, 313V, 331Y | IESNHRVYLG HGAGVPIPLL DALVARAPEL |
| [Roseiflexus castenholzii | 61 AGEDPTTAPE LAASFRHNAL FIGHNTRRAV | | RNVEIIQMLT |
| DSM 13941] | NEGRADFTPI FLGEIPKLFR QGILPLDVAM | | 61 AGEDPTTAPE LAASFRHNAL |
| Sequence ID: | 121 IQVSPPDRHG FCSLGVEVGC TLPAARTAKI | | FIGHNTRRAV NEGRADFTPI FLGEIPYLFR |
| ref\|YP_001433830.1\| | VIAEVNARMP RTLGDSFIHI SRLTALVESD | | QGILPLDVAM |
| Length: 431 | 181 RPLLELPQGE TNSVAQAIGR HIAELIPDGA | | 121 IQVSPPDRHG FCSLGVEVGC |
| | TLQLGIGAIP DAVLSNLHGK RHLGIHTELF | | TLPAARTAKI VIAEVNARMP RTLGDSFIHI |
| | 241 SDGVIDLVEA GVIDGELKTI HQGKVVAGFI | | SRLTALVESD |
| | LGSQRCFDWA HNNAMVEMHP TDYVNDPFVI | | 181 RPLLELPQGE TNSVAQAIGR |
| | 301 AQHKNMVAVN SALQVDLTGQ VCADSIGTRL | | HIAELIPDGA TLQLGIGAIP DAVLSNLHGK |
| | YSGVGGQVDF IRGASRSEGG IPIIAISSTA | | RHLGIHTELF |
| | 361 RDGTISRIVP TLDVGAGVVT SRYDVHFVVT | | 241 SDGVIDLVEA GVIDGELKTI |
| | EYGVADLYGR TLAQRARALI NIAHPAFRDQ | | HQGKVVAGFI LGSQRCFDWA HNNAMVEMHP |
| | 421 LTEAAKKLHY I (SEQ ID NO: 189) | | TDYVNDPFVI |
| | | | 301 AQHKNMVAVN SAVQVDLTGQ |
| | | | VCADSIGTRL YSGVGGQVDF IRGASRSEGG |
| | | | IPIIAISSTA |
| | | | 361 RDGTISRIVP TLDVGAGVVT |
| | | | SRYDVHFVVT EYGVADLYGR TLAQRARALI |
| | | | NIAHPAFRDQ |
| | | | 421 LTEAAKKLHY I (SEQ ID NO: 200) |
| hypothetical protein | 1 MNWQEEYKRR LCSAEEAVQH IHSGDTVFLG | 29L, 31G, 33A, 57Q, 107Y, | 1 MNWQEEYKRR LCSAEEAVQH |
| HMPREF0995_05302 | HAVAESSLLV DAMVQNATAY QDVRIIHMTN | 230R, 310S, 312V, 330Y | IHSGDTVFLG GAAAESSLLV DAMVQNATAY |
| [Lachnospiraceae | 61 LGSGAYTQPG MESHFHVSPF FLGATVRAAV | | QDVRIIQMTN |
| bacterium 7_1_58FAA] | AEGRGDFIPC FFHEIPQLIR ERRVPCDVLM | | 61 LGSGAYTQPG MESHFHVSPF |
| Sequence ID: | 121 TQVSPSDEKG YCSLGVSADY TYQALKSART | | FLGATVRAAV AEGRGDFIPC FFHEIPYLIR |
| ref\|ZP_09534466.1\| | VIAHVNDQYP YTFGTKVHVT ELDYIVEQSA | | ERRVPCDVLM |
| Length: 436 | 181 PLYESKPPKI GEVEEKIGRC CASLIRDGAT | | 121 TQVSPSDEKG YCSLGVSADY |
| | LQLGIGAIPD AVMMFLGDKH DLGIHSEMIS | | TYQALKSART VIAHVNDQYP YTFGTKVHVT |
| | 241 DGTMELYQKG VITNQRKNLD RGKMTITFAM | | ELDYIVEQSA |
| | GTKKLYQFLD KNPDVLVKPV DYVNHPVTIM | | 181 PLYESKPPKI GEVEEKIGRC CASLIRDGAT |
| | 301 KEHDMISINS AIQVDLQGQV DAEAIGLRQF | | LQLGIGAIPD AVMMFLGDKR DLGIHSEMIS |
| | SGVGGQVDFI RGAAMGGGTS IIAMPSTTQK | | 241 DGTMELYQKG VITNQRKNLD |
| | 361 KDGTLISKIV PFLDQGAPVT TSRNDVDYII | | RGKMTITFAM GTKKLYQFLD KNPDVLVKPV |
| | TEYGIAHLKG ETLRDRAWAL ISISHPSVRD | | DYVNHPVTIM |
| | 421 SLAEEFERRF HQNYRA (SEQ ID NO: 190) | | 301 KEHDMISINS AVQVDLQGQV |
| | | | DAEAIGLRQY SGVGGQVDFI RGAAMGGGTS |
| | | | IIAMPSTTQK |
| | | | 361 KDGTLISKIV PFLDQGAPVT TSRNDVDYII |
| | | | TEYGIAHLKG ETLRDRAWAL ISISHPSVRD |
| | | | 421 SLAEEFERRF HQNYRA (SEQ ID NO: 201) |
| 4-hydroxybutyrate | 1 MSKNWREVYK DKIVTADEAV KQIKSGDRLI | 31L, 33G, 35A, 59Q, 109Y, | 1 MSKNWREVYK DKIVTADEAV |
| coenzyme A transferase | FSHACGEAQE ITDALLRNKE SYEKVEIIHL | 234R 314S, 316V, 334Y | KQIKSGDRLI LSGAAGEAQE ITDALLRNKE |
| [Fusobacterium | 61 VPMGKGEYAQ EENQKYFYHN SFFGGGSTRK | | SYEKVEIIQL |
| necrophorum subsp. | AINGTYGDFT PSFFFEIPNL FRKNGKLPLD | | 61 VPMGKGEYAQ EENQKYFYHN |
| funduliforme ATCC | 121 VAIIQVSAPD EHGYCSYGIS CDYTKGAAEN | | SFFGGGSTRK AINGTYGDFT PSFFFEIPYL |
| 51357] | AKIVIAQVNK YMPRTLGENF THLSAIDSIV | | FRKNGKLPLD |
| Sequence ID: | 181 EYDQPILQLN PPKIGEVEKK IGEYCAGLIQ | | 121 VAIIQVSAPD EHGYCSYGIS |
| ref\|ZP_14365187.1\| | DGDTLQLGIG AIPDAVLTFL KEKKHLGIHS | | CDYTKGAAEN AKIVIAQVNK YMPRTLGENF |
| Length: 436 | 241 EMISDGVVDL ILAGVIDNSQ KTIHKNKCIV | | THLSAIDSIV |
| | SFLMGSQKLY DYVHNNPGVE LYPVDYVNHP | | 181 EYDQPILQLN PPKIGEVEKK |
| | 301 FVIAQNDNMV SINSALQVDL | | IGEYCAGLIQ DGDTLQLGIG AIPDAVLTFL |
| | MGQVNAESMG AKQFSGTGGQ VDFVRGAAMS | | KEKRHLGIHS |
| | KGGRSIIAMP | | 241 EMISDGVVDL ILAGVIDNSQ |
| | 361 STAAKGTISK IVMNLDVGAT VTTSRNDVDY | | KTIHKNKCIV SFLMGSQKLY DYVHNNPGVE |
| | IITEYGIAEL KGKTLRERAK ALIAIAHPDF | | LYPVDYVNHP |
| | 421 REQLTKQALE KFQRLE (SEQ ID NO: 191) | | 301 FVIAQNDNMV SINSAVQVDL |
| | | | MGQVNAESMG AKQYSGTGGQ VDFVRGAAMS |
| | | | KGGRSIIAMP |
| | | | 361 STAAKGTISK IVMNLDVGAT |
| | | | VTTSRNDVDY IITEYGIAEL KGKTLRERAK |
| | | | ALIAIAHPDF |
| | | | 421 REQLTKQALE KFQRLE (SEQ ID NO: 202) |
| 4-hydroxybutyrate | 1 MEWEEIYKEK LVTAEKAVSK IENHSRVVFA | 29L, 31G, 33A, 57Q, 107Y, | 1 MEWEEIYKEK LVTAEKAVSK IENHSRVVLA |
| coenzyme A transferase | HAVGEPVDLV NALVKNKDNY IGLEIVHMVA | 231R, 311S, 313V, 331Y | GAAGEPVDLV NALVKNKDNY IGLEIVQMVA |
| [Clostridium kluyveri | 61 MGKGVYTKEG MQRHFRHNAL FVGGSTRDAV | | 61 MGKGVYTKEG MQRHFRHNAL |
| DSM 555] | NSGRAVYTPC FFYEVPSLFK EKRLPVDVAL | | FVGGSTRDAV NSGRAVYTPC FFYEVPYLFK |
| Sequence ID: | 121 IQVSEPDKYG YCSFGVSNDY TKPAAESAKL | | EKRLPVDVAL |
| gb\|AAA92344.1\|Length: | VIAEVNKNMP RTLGDSFIHV SDIDYIVEAS | | 121 IQVSEPDKYG YCSFGVSNDY |
| 429 | 181 HPLLELQPPK LGDVEKAIGE NCASLIEDGA | | TKPAAESAKL VIAEVNKNMP RTLGDSFIHV |
| | TLQLGIGAIP DAVLLFLKNK KNLGIHSEMI | | SDIDYIVEAS |
| | 241 SDGVMELVKA GVINNKKKTL HPGKIVVTFL | | 181 HPLLELQPPK LGDVEKAIGE |
| | MGTKKLYDFV NNNPMVETYS VDYVNNPLVI | | NCASLIEDGA TLQLGIGAIP DAVLLFLKNK |
| | 301 MKNDNMVSIN SCVQVDLMGQ VCSESIGLKQ | | RNLGIHSEMI |
| | ISGVGGQVDF IRGANLSKGG KAIIAIPSTA | | 241 SDGVMELVKA GVINNKKKTL |
| | 361 GKGKVSRITP LLDTGAAVTT SRNEVDYVVT | | HPGKIVVTFL MGTKKLYDFV NNNPMVETYS |
| | EYGVAHLKGK TLRNRARALI NIAHPKFRES | | VDYVNNPLVI |
| | 421 LMNEFKKRF (SEQ ID NO: 192) | | 301 MKNDNMVSIN SCVQVDLMGQ |
| | | | VCSESIGLKQ YSGVGGQVDF IRGANLSKGG |
| | | | KAIIAIPSTA |
| | | | 361 GKGKVSRITP LLDTGAAVTT |
| | | | SRNEVDYVVT EYGVAHLKGK TLRNRARALI |
| | | | NIAHPKFRES |
| | | | 421 LMNEFKKRF (SEQ ID NO: 203) |
| 4-Hydroxybutyrate CoA- | 1 MDWKKIYEDR TCTADEAVKS IKSGDRVLFA | 29L, 31G, 33A, 57Q, S107Y, | 1 MDWKKIYEDR TCTADEAVKS |
| transferase [Clostridium | HCVAEPPVLV EAMVANAAAY KNVTVSQMVT | 231R, 311S, 313V, 331Y | IKSGDRVLLA GCAAEPPVLV EAMVANAAAY |
| acetobutyricum] | 61 LGKGEYSKPE YKENFTFEGW FTSPSTRGSI | | KNVTVSQMVT |
| | AEGHGQFVPV FFHEVPSLIR KDIFHVDVFM | | 61 LGKGEYSKPE YKENFTFEGW |
| | 121 MVSPPDHNGF CCVGVSSDYT MQAIKSAKIV | | FTSPSTRGSI AEGHGQFVPV FFHEVPYLIR |
| | LAEVNDQVPV VYGDTFVHVS EIDKFVETSH | | KDIFHVDVFM |
| | 181 PLPEIGLPKI GEVEAAIGKH CASLIEDGST | | 121 VMVSPPDHNG FCCVGVSSDY |
| | LQLGIGAIPD AVLSQLKDKR HLGIHSEMIS | | TMQAIKSAKI VLAEVNDQVP VVYGDTFVHV |
| | 241 DGVVDLYEAG VIDCSQKSID KGKMAITFLM | | SEIDKFVETS |
| | GTKRLYDFAA NNPKVELKPV DYINHPSVVA | | 181 HPLPEIGLPK IGEVEAAIGK HCASLIEDGS |
| | 301 QCSKMVCINS CLQVDFMGQI VSDSIGTKQY | | TLQLGIGAIP DAVLSQLKDK RHLGIHSEMI |
| | SGVGGQVDFV RGASMSIDGK GKAIIAMPSV | | 241 SDGVVDLYEA GVIDCSQKSI |
| | 361 AKKKDGSMIS KIVPFIDHGA AVTTSRNDAD | | DKGKMAITFL MGTKRLYDFA ANNPKVELKP |
| | YVVTEYGIA MKGKSLQDRA RALINIAHPD | | VDYINHPSVV |
| | 421 FKDELKAEFE KRFNAAF (SEQ ID NO: 193) | | 301 AQCSKMVCIN SCVQVDFMGQ |
| | | | IVSDSIGTKQ YSGVGGQVDF VRGASMSIDG |
| | | | KGKAIIAMPS |
| | | | 361 VAKKKDGSMI SKIVPFIDHG |
| | | | AAVTTSRNDA DYVVTEYGIA EMKGKSLQDR |
| | | | ARALINIAHP |
| | | | 421 DFKDELKAEF EKRFNAAF (SEQ ID |
| | | | NO: 204) |
| acetyl-CoA | 1 MSKISWKDLY KSKVVTADEA VRKIKSNDRV | 32L, 34G, 36A, 57Q, 110Y, | 1 MSKISWKDLY KSKVVTADEA |
| hydrolase/transferase | VTGHACGEPK EIIDAMVRNK DLYENVEIVH | 231R, 314S, 316V, 334Y | VRKIKSNDRV VLGGAAGEPK EIIDAMVRNK |
| [Clostridium beijerinckii | 61 MVSMGKSEYC KPEMAVNFRH NSIFAGGTTR | | DLYENVEIVQ |
| NCIMB 8052] | EAIFDGRADF TPCFFSEVPK MFREGTLPVD | | 61 MVSMGKSEYC KPEMAVNFRH |
| Sequence ID: | 121 VALVQLSVPD EHGYCSFGVS NDYTKPAAEA | | NSIFAGGTTR EAIFDGRADF TPCFFSEVPY |
| ref\|YP_001309226.1 | AKIVIAELNE KMPRTLGDSF IHVSDIDYIV | | MFREGTLPVD |
| Length: 436 | 181 ETSNDIIELK PPKIGEVEKA IGENCAKLIE | | 121 VALVQLSVPD EHGYCSFGVS |
| | DGSTLQLGIG AIPDAVLLFL KGKKDLGIHS | | NDYTKPAAEA AKIVIAELNE KMPRTLGDSF |
| | 241 EMISDGVVEL IEAGVITNKA KTLHPGKSVV | | IHVSDIDYIV |
| | TFLMGTKRLY DYVNGNPSVA MYPVDYVNNP | | 181 ETSNDIIELK PPKIGEVEKA IGENCAKLIE |
| | 301 CVIAENYKMV SINSCIQVDL MGQVAADTIG | | DGSTLQLGIG AIPDAVLLFL KGKRDLGIHS |
| | LKQFSGVGGQ VDFVRGAAMA KGGKSIIAMP | | 241 EMISDGVVEL IEAGVITNKA |
| | 361 STASKGKLSR IVPILDEGAT VTTSRNDIHY | | KTLHPGKSVV TFLMGTKRLY DYVNGNPSVA |
| | VVTEFGIAEL KGKTLKERAK ALINVAHPDF | | MYPVDYVNNP |
| | 421 RDALIKEWEK RFKVKF (SEQ ID NO: 194) | | 301 CVIAENYKMV SINSCVQVDL |
| | | | MGQVAADTIG LKQYSGVGGQ VDFVRGAAMA |
| | | | KGGKSIIAMP |
| | | | 361 STASKGKLSR IVPILDEGAT VTTSRNDIHY |
| | | | VVTEFGIAEL KGKTLKERAK ALINVAHPDF |
| | | | 421 RDALIKEWEK RFKVKF (SEQ ID NO: 205) |
| 4-hydroxybutyrate CoA- | 1 MDWKKIYEEK RCTAKEAVQS IKSNDRVVFA | 29L, 31G, 33A, 57Q, 107Y, | 1 MDWKKIYEEK RCTAKEAVQS |
| transferase [Eubacterium | HAVAEPTSLV DAMVENKEAY ENVTISHMFS | 231R, 311S, 313V, 331Y | IKSNDRVVLA GAAAEPTSLV DAMVENKEAY |
| saphenum ATCC 49989] | 61 FGKGEYSLPE NSKHFRYEGW FNSPNTRKST | | ENVTISQMFS |
| Sequence ID: | ELGHGDYVPA FFHQVPSLIR RGVLPVDVVM | | 61 FGKGEYSLPE NSKHFRYEGW |
| ref\|ZP_05427217.1\| | 121 VSLSKPDAHG YCSTGVSSDY TMQAIKSAKV | | FNSPNTRKST ELGHGDYVPA FFHQVPYLIR |
| Length: 438 | VLAEINDQVP KTFGESFVHI SEIDKLVEHS | | RGVLPVDVVM |
| | 181 HPLFELGLPK IGEVEEAIGK HCASLIEDGA | | 121 VSLSKPDAHG YCSTGVSSDY |
| | TLQLGIGAIP DAVLAQLKHK KDLGIHSEMI | | TMQAIKSAKV VLAEINDQVP KTFGESFVHI |
| | 241 SDGAVELFEA GVINNSKKSI DKGKMVVTFL | | SEIDKLVEHS |
| | MGTKRLYDFV NENPAVELRP VDYVNHPEVI | | 181 HPLFELGLPK IGEVEEAIGK HCASLIEDGA |
| | 301 AKSSNLVCIN ACLQVDFMGQ VVSDTIGTRQ | | TLQLGIGAIP DAVLAQLKHK RDLGIHSEMI |
| | FSGVGGQVDF VRGAAMAHDE KAKAIIAMPS | | 241 SDGAVELFEA GVINNSKKSI |
| | 361 VAVKKDGTKI SKIVPFIDHG AAVTTSRHDT | | DKGKMVVTFL MGTKRLYDFV NENPAVELRP |
| | DYIVTEYGIA EMVGKTMKDR ARSLINIAHP | | VDYVNHPEVI |
| | 421 DFRDELKAEF EKRFNVKF (SEQ ID NO: 195) | | 301 AKSSNLVCIN SCLQVDFMGQ |
| | | | VVSDTIGTRQ YSGVGGQVDF VRGAAMAHDE |
| | | | KAKAIIAMPS |
| | | | 361 VAVKKDGTKI SKIVPFIDHG |
| | | | AAVTTSRHDT DYIVTEYGIA EMVGKTMKDR |
| | | | ARSLINIAHP |
| | | | 421 DFRDELKAEF EKRFNVKF (SEQ ID |
| | | | NO: 206) |
| 4-hydroxybutyrate | 1 MSWQELYQQK RMTAEQAVSH IKSGDRVVVA | 29L, 31G, 33A, 57Q, 107Y, | 1 MSWQELYQQK RMTAEQAVSH |
| coenzyme A transferase | HATGEPSALI DAMVKNADAY RNVEIVHMVA | 230R, 310S, 312V, 331Y | IKSGDRVVLA GAAGEPSALI DAMVKNADAY |
| [Clostridium sp. MSTE9] | 61 MGKGEYCLPE YTENFRHNSL FLGASTRDAI | | RNVEIVQMVA |
| Sequence ID: | AKGRGDFTPV YFSQIPDLLR EELHPNVALL | | 61 MGKGEYCLPE YTENFRHNSL |
| ref\|ZP_14663205.1\| | 121 HLSPPDEHGY CSYGVSVDYT KPAAEIADLR | | FLGASTRDAI AKGRGDFTPV YFSQIPYLLR |
| Length: 433 | IAQINPNMPR TLGDSFIHVS ELDCIVEVDD | | EELHPNVALL |
| | 181 PIIELGQAKI GETECAIGEH CASLIQDGDC | | 121 HLSPPDEHGY CSYGVSVDYT |
| | LQLGIGAIPD AVLLFLKEKK DLGIHSEMFS | | KPAAEIADLR IAQINPNMPR TLGDSFIHVS |
| | 241 DGVVELVEAG VITNRRKTLH KGQCVVTFLM | | ELDCIVEVDD |
| | GSKRLYDYVN NNPSVAMYPV DYVNNPAVIA | | 181 PIIELGQAKI GETECAIGEH CASLIQDGDC |
| | 301 KNDNLVSINS CIQVDLMGQV VSDSIGPRQF | | LQLGIGAIPD AVLLFLKEKR DLGIHSEMFS |
| | SGVGGQVDFV RGANLSRGGK SIMAMPSSLV | | 241 DGVVELVEAG VITNRRKTLH |
| | 361 KKDGTRVSKI AVQIDAGAAV TTSRYDVNYI | | KGQCVVTFLM GSKRLYDYVN NNPSVAMYPV |
| | VTEFGVAKLK GKTLRDRAKA LIEIAHPDFR | | DYVNNPAVIA |
| | 421 EELMEEFHRR FGV (SEQ ID NO: 196) | | 301 KNDNLVSINS CVQVDLMGQV |
| | | | VSDSIGPRQY SGVGGQVDFV RGANLSRGGK |
| | | | SIMAMPSSLV |
| | | | 361 KKDGTRVSKI AVQIDAGAAV |
| | | | TTSRYDVNYI VTEFGVAKLK GKTLRDRAKA |
| | | | LIEIAHPDFR |
| | | | 421 EELMEEFHRR FGV (SEQ ID NO: 207) |
| 4-Hydroxybutyrate CoA- | 1 MDWKKIYEDR TCTADEAVKS IKSGDRVLFA | 29L, 31G, 33A, 57Q, 107Y, | 1 MDWKKIYEDR TCTADEAVKS |
| transferase [Clostridium | HCVAEPPVLV EAMVANAAAY KNVTVSHMVT | 231R, 311S, 313V, 331Y | IKSGDRVLLA GCAAEPPVLV EAMVANAAAY |
| aminobutyricum] | 61 LGKGEYSKPE YKENFTFEGW FTSPSTRGSI | | KNVTVSQMVT |
| GenBank: CAB60036.2 | AEGHGQFVPV FFHEVPSLIR KDIFHVDVFM | | 61 LGKGEYSKPE YKENFTFEGW |
| | 121 VMVSPPDHNG FCCVGVSSDY TMQAIKSAKI | | FTSPSTRGSI AEGHGQFVPV FFHEVPYLIR |
| | VLAEVNDQVP VVYGDTFVHV SEIDKFVETS | | KDIFHVDVFM |
| | 181 HPLPEIGLPK IGEVEAAIGK HCASLIEDGS | | 121 VMVSPPDHNG FCCVGVSSDY |
| | TLQLGIGAIP DAVLSQLKDK KHLGIHSEMI | | TMQAIKSAKI VLAEVNDQVP VVYGDTFVHV |
| | 241 SDGVVDLYEA GVIDCSQKSI DKGKMAITFL | | SEIDKFVETS |
| | MGTKRLYDFA ANNPKVELKP VDYINHPSVV | | 181 HPLPEIGLPK IGEVEAAIGK HCASLIEDGS |
| | 301 AQCSKMVCIN ACLQVDFMGQ IVSDSIGTKQ | | TLQLGIGAIP DAVLSQLKDK RHLGIHSEMI |
| | FSGVGGQVDF VRGASMSIDG KGKAIIAMPS | | 241 SDGVVDLYEA GVIDCSQKSI |
| | 361 VAKKKDGSMI SKIVPFIDHG AAVTTSRNDA | | DKGKMAITFL MGTKRLYDFA ANNPKVELKP |
| | DYVVTEYGIA EMKGKSLQDR ARALINIAHP | | VDYINHPSVV |
| | 421 DFKDELKAEF EKRFNAAF (SEQ ID NO: 197) | | 301 AQCSKMVCIN SCVQVDFMGQ |
| | | | IVSDSIGTKQ FSGVGGQVDF VRGASMSIDG |
| | | | KGKAIIAMPS |
| | | | 361 VAKKKDGSMI SKIVPFIDHG |
| | | | AAVTTSRNDA DYVVTEYGIA EMKGKSLQDR |
| | | | ARALINIAHP |
| | | | 421 DFKDELKAEF EKRFNAAF (SEQ ID |
| | | | NO: 208) |
| acetyl-CoA | 1 MDWKKLYKSK LVSAKEAVSN IKSNSRVVVS | 29L, 31G, 33A, 57Q, 107Y, | 1 MDWKKLYKSK LVSAKEAVSN |
| hydrolase/transferase | IAVAEPTELI DALVANKENY ENVEVVHMVD | 231R, 311S, 313V, 331Y | IKSNSRVVVS IAAAEPTELI DALVANKENY |
| [Clostridium | 61 MGKSEYAQEG MEKYFKYNSI FVGASTKDAV | | ENVEVVQMVD |
| carboxidivorans P7] | NSGRSDFTPC CFYELPRLFE EGYLPVDVVL | | 61 MGKSEYAQEG MEKYFKYNSI |
| Sequence ID: | 121 IQVSKPDKHG YCSFGVSNDY TKPAADCAKM | | FVGASTKDAV NSGRSDFTPC CFYELPRLFE |
| ref\|ZP_05395303.1\| | VIAEVNENMP RVLGDSFIHI SDIDYIVETS | | EGYLPVDVVL |
| Length: 433 | 181 HPIMELKQPK IGKIEEAIGE YCASLIEDGS | | 121 IQVSKPDKHG YCSFGVSNDY |
| | TLQLGIGAIP DAVLLFLKDK KDLGIHSEMI | | TKPAADCAKM VIAEVNENMP RVLGDSFIHI |
| | 241 SDGVVDLVES GVINNKEKTL NPGKIVVTFF | | SDIDYIVETS |
| | MGTKKLYDFI DDNPMVESYP VSYVNDPTVI | | 181 HPIMELKQPK IGKIEEAIGE YCASLIEDGS |
| | 301 MKNSKMISIN SCVEVDLMGQ VCSESIGMNQ | | TLQLGIGAIP DAVLLFLKDK RDLGIHSEMI |
| | ISGIGGQVDF IRGANMCKDG KAIIAIPSTA | | 241 SDGVVDLVES GVINNKEKTL |
| | 361 AKGKVSRIVP LIEKGTPITT SRTDVDYIIT | | NPGKIVVTFF MGTKKLYDFI DDNPMVESYP |
| | EYGIARLKSK SLKERARALI NIAHPDFRAW | | VSYVNDPTVI |
| | 421 LIDEYEKRFK TKF (SEQ ID NO: 198) | | 301 MKNSKMISIN SCVEVDLMGQ |
| | | | VCSESIGMNQ ISGIGGQVDF IRGANMCKDG |
| | | | KAIIAIPSTA |
| | | | 361 AKGKVSRIVP LIEKGTPITT SRTDVDYIIT |
| | | | EYGIARLKSK SLKERARALI NIAHPDFRAW |
| | | | 421 LIDEYEKRFK TKF (SEQ ID NO: 209) |

Exemplary cat2 variants include, but are not limited to, single substitutions, or a combination of one or more of the substitutions, at 29L, 31G, 33A, 57Q, 107Y, 231R, 311S, 313V, 331Y for representative sequence(s) shown in Table 54; 29L, 31G, 33A, 57Q, 107Y, 230R, 310S, 312V, 330Y for representative sequence(s) shown in Table 54; 31L, 33G, 35A, 59Q, 109Y, 234R 314S, 316V, 334Y for representative sequence(s) shown in Table 54; 32L, 34G, 36A, 57Q, 110Y, 231R, 314S, 316V, 334Y for representative sequence(s) shown in Table 54; 29L, 31G, 33A, 57Q, 107Y, 230R, 310S, 312V, 331Y for representative sequence(s) shown in Table 54. Representative combinations include combinations of the single substitutions exemplified in Table 54 as in the corresponding respective sequences, including but not limited to: 314S, 316V, 29L, 33A, 57Q; 231R, 331Y; 57Q, 231R; 57Q, 311S, 57Q, 331Y, 33A, 57Q; 231R, 311S; 311S, 331Y, 231R, 311S, 331Y, 107Y, 231R, 331Y, 57Q, 231R, 311S; 57Q, 311S, 331Y, 57Q, 231R, 311S, 331Y, 31G, 33A, 331Y; 33A, 231R, 331Y, 57Q, 231R, 331Y, 31G, 33A, 331Y; 33A, 331Y, 33A, 57Q, 107Y, 107Y, 310S, 312V, 331Y, 57Q, 231S, 311S, F331Y, 29L, 31G, 33A, 57Q, 107Y, 231R 311S, 313V, 331Y, 109Y, 234R 314S; 59Q, 109Y, 314S, 316V; 31G, 57Q, and the like. It is readily apparent that these an other combinations can be readily made, used and screened using methods well known in the art and as disclosed herein. Based on methods well known in the art for aligning homologous sequences, as described herein, the corresponding positions for the indicated variant amino acid positions of Table 54 can be readily determined for a homologous cat2 sequence.

Variants of ADH were generated using site-directed mutagenesis and/or saturation mutagenesis and/or error-prone PCR and/or directed evolution. Variants were screened for production of products and/or byproducts such as BDO or ethanol in high-throughput assays or in in vitro assays. Additional variants were generated by random combinations of mutations. An exemplary screening method for ADH activity includes, for example, the above in vivo methods for measuring BDO and/or ethanol production. For in vitro assays, purified enzymes were assayed using 96 well polystyrene plates with 100 μl assay mixture that contained 50 mM Tris, pH 7.4, 150 mM NaCl, 5 mM DTT, 10 mM histidine, 0.3 mM NADPH at varied concentrations of the substrate 4-hydroxybutyraldehyde. The reaction was initiated with addition of purified enzyme and the reduction of NADPH was monitored at 340 nm. Certain enzyme variants exhibited activity of about 1.5-fold, about 2-fold, about 2.5-fold, about 3-fold, about 3.5-fold, about 5-fold, about 6-fold, and about 9-fold higher than wild type. Variants with combinations of two or more variant amino acid positions exhibited activities greater than wild type. Generating enzyme variants by combining active variant amino acid positions resulted in enzyme variants with improved properties.

Table 55 provides exemplary ADH sequences based on homology. One skilled in the art will readily understand that such sequences can be analyzed with routine and well known methods for aligning sequences (for example BLAST, blast.ncbi.nlm.nih.gov; Altschul et al., “J. Mol. Biol. 215:403410 (1990)). Such alignments can provide information on conserved residues that can be utilized to identify a consensus sequence for preserving enzyme activity as well as positions for generating further enzyme variants.

TABLE 55
Exemplary ADH sequences.

| Description | Accession No. |
|---|---|
| NADPH-dependent butanol dehydrogenase [Clostridium novyi | YP_878939.1 |
| NT] >gb\|ABK62471.1\| NADPH-dependent butanol dehydrogenase [Clostridium | GI: 118444971 |
| novyi NT] | (SEQ ID NO: 339) |
| NADPH-dependent butanol dehydrogenase [Clostridium perfringens NCTC | ZP_02642725.2 |
| 8239] >gb\|EDT78274.1\| NADPH-dependent butanol dehydrogenase [Clostridium | GI: 182420493 |
| perfringens NCTC 8239] | (SEQ ID NO: 340) |
| hypothetical protein DORFOR_01493 [Dorea formicigenerans ATCC | ZP_02234621.1 |
| 27755] >gb\|EDR47002.1\| alcohol dehydrogenase, iron-dependent [Dorea | GI: 166031792 |
| formicigenerans ATCC 27755] | (SEQ ID NO: 341) |
| NADPH-dependent butanol dehydrogenase [Clostridium hathewayi DSM | ZP_06115415.1 |
| 13479] >gb\|EFC98086.1\| NADPH-dependent butanol dehydrogenase [Clostridium | GI: 266622480 |
| hathewayi DSM 13479] | (SEQ ID NO: 342) |
| iron-containing alcohol dehydrogenase [Clostridiales bacterium | ZP_04668388.1 |
| 1_7_47_FAA] >gb\|EEQ59453.1\| iron-containing alcohol dehydrogenase [Clostridiales | GI: 239625357 |
| bacterium 1_7_47FAA] | (SEQ ID NO: 343) |
| NADPH-dependent butanol dehydrogenase [Clostridium celatum DSM | ZP_19299687.1 |
| 1785] >gb\|EKY22101.1\| NADPH-dependent butanol dehydrogenase [Clostridium | GI: 429767490 |
| celatum DSM 1785] | (SEQ ID NO: 344) |
| RecName: Full = NADPH-dependent butanol dehydrogenase; Short = | P13604.1 |
| BDH >pir\|JU0053 NADPH-dependent butanol dehydrogenase (EC 1.1.1.-)- Clostridium | GI: 113352 |
| acetobutylicum >gb\|AAA83520.1\| NADPH-dependent butanol dehydrogenase | (SEQ ID NO: 345) |
| [Clostridium saccharobutylicum] | |
| Alcohol dehydrogenase, class IV [Ruminococcus bromii L2-63] >emb\|CBL15420.1\| | YP_007781197.1 |
| Alcohol dehydrogenase, class IV [Ruminococcus bromii L2-63] | GI: 479151021 |
| | (SEQ ID NO: 346) |
| NADH-dependent butanol dehydrogenase [Clostridium botulinum NCTC | ZP_02614964.1 |
| 2916] >gb\|EDT80777.1\| NADH-dependent butanol dehydrogenase [Clostridium | GI: 168180300 |
| botulinum NCTC 2916] | (SEQ ID NO: 347) |
| NADPH-dependent butanol dehydrogenase [Clostridium butyricum | ZP_02950013.1 |
| 5521] >ref\|ZP_04529706.1\| NADH-dependent butanol dehydrogenase [Clostridium | GI: 182418740 |
| butyricum E4 str. BoNT E BL5262] >gb\|EDT74990.1\| NADPH-dependent butanol | (SEQ ID NO: 348) |
| dehydrogenase [Clostridium butyricum 5521] >gb\|EEP52366.1\| NADH-dependent | |
| butanol dehydrogenase [Clostridium butyricum E4 str. BoNT E BL5262] | |
| NADH-dependent butanol dehydrogenase [Clostridium sp. JC122] | ZP_10929454.1 |
| | GI: 403387397 |
| | (SEQ ID NO: 349) |
| Alcohol dehydrogenase, class IV [butyrate-producing bacterium | YP_007790855.1 |
| SSC/2] >emb\|CBL39860.1\| Alcohol dehydrogenase, class IV [butyrate-producing | GI: 479161586 |
| bacterium SSC/2] | (SEQ ID NO: 350) |
| NADH-dependent butanol dehydrogenase [Clostridium botulinum A3 str. Loch | YP_001787047.1 |
| Maree] >gb\|ACA54890.1\| NADH-dependent butanol dehydrogenase [Clostridium | GI: 170759490 |
| botulinum A3 str. Loch Maree] | (SEQ ID NO: 351) |
| NADH-dependent butanol dehydrogenase [Clostridium botulinum | ZP_02619369.1 |
| Bf] >ref\|YP_002862536.1\| NADH-dependent butanol dehydrogenase [Clostridium | GI: 168184705 |
| botulinum Ba4 str. 657] >gb\|EDT84181.1\| NADH-dependent butanol dehydrogenase | (SEQ ID NO: 352) |
| [Clostridium botulinum Bf] >gb\|ACQ52130.1\| NADH-dependent butanol | |
| dehydrogenase [Clostridium botulinum Ba4 str. 657] | |
| Adh1 [Clostridium kluyveri DSM 555] >gb\|EDK32597.1\| Adh1 [Clostridium | YP_001393945.1 |
| kluyveri DSM 555] | GI: 153953180 |
| | (SEQ ID NO: 353) |
| hypothetical protein CLOLEP_02428 [Clostridium leptum DSM | ZP_02080962.1 |
| 753] >gb\|EDO60823.1\| NADPH-dependent butanol dehydrogenase [Clostridium | GI: 160933574 |
| leptum DSM 753] | (SEQ ID NO: 354) |
| NADH-dependent butanol dehydrogenase [Clostridium botulinum El str. ‘BoNTE | ZP_04822415.1 |
| Beluga’] >gb\|EES49700.1\| NADH-dependent butanol dehydrogenase [Clostridium | GI: 251779495 |
| botulinum E1 str. ‘BoNT E Beluga’] | (SEQ ID NO: 355) |
| NADPH-dependent butanol dehydrogenase [Clostridium perfringens B str. ATCC | ZP_02635244.2 |
| 3626] >gb\|EDT24389.1\| NADPH-dependent butanol dehydrogenase [Clostridium | GI: 170763924 |
| perfringens B str. ATCC 3626] | (SEQ ID NO: 356) |
| NADPH-dependent butanol dehydrogenase [Clostridium perfringens | YP_698587.1 |
| SM101] >gb\|ABG87717.1\| NADPH-dependent butanol dehydrogenase [Clostridium | GI: 110803846 |
| perfringens SM101] | (SEQ ID NO: 357) |
| putative NADPH-dependent butanol dehydrogenase [Clostridium carboxidivorans | ZP_06853946.1 |
| P7] >gb\|EFG89794.1\| putative NADPH-dependent butanol dehydrogenase [Clostridium | GI: 296185536 |
| carboxidivorans P7] | (SEQ ID NO: 358) |
| NADPH-dependent butanol dehydrogenase [Clostridium ljungdahlii DSM | YP_003780648.1 |
| 13528] >gb\|ADK15546.1\| predicted NADPH-dependent butanol dehydrogenase | GI: 300855664 |
| [Clostridium ljungdahlii DSM 13528] | (SEQ ID NO: 359) |
| NADPH-dependent butanol dehydrogenase [Clostridium citroniae WAL- | ZP_09058579.1 |
| 17108] >gb\|EHE99483.1\| NADPH-dependent butanol dehydrogenase [Clostridium | GI: 355672858 |
| citroniae WAL-17108] | (SEQ ID NO: 360) |
| NADPH-dependent butanol dehydrogenase [Lachnospiraceae bacterium | ZP_08331580.1 |
| 6_1_63FAA] >gb\|EGG80541.1\| NADPH-dependent butanol dehydrogenase | GI: 331082454 |
| [Lachnospiraceae bacterium 6_1_63FAA] | (SEQ ID NO: 361) |
| hypothetical protein CLOSPO_03272 [Clostridium sporogenes ATCC | ZP_02996149.1 |
| 15579] >gb\|EDU37103.1\| alcohol dehydrogenase, iron-dependent [Clostridium | GI: 187779676 |
| sporogenes ATCC 15579] | (SEQ ID NO: 362) |
| NADPH-dependent butanol dehydrogenase [Clostridium perfringens E str. | ZP_02631505.2 |
| JGS1987] >gb\|EDT15707.1\| NADPH-dependent butanol dehydrogenase [Clostridium | GI: 170764079 |
| perfringens E str. JGS1987] | (SEQ ID NO: 363) |
| iron-containing alcohol dehydrogenase [Clostridium saccharoperbutylacetonicum N1- | YP_007455800.1 |
| 4(HMT)] >gb\|AGF56546.1\| iron-containing alcohol dehydrogenase [Clostridium | GI: 451819599 |
| saccharoperbutylacetonicum N1-4(HMT)] | (SEQ ID NO: 364) |
| hypothetical protein HMPREF9476_00911 [Clostridium perfringens WAL- | ZP_16426838.1 |
| 14572] >gb\|EHP49458.1\| hypothetical protein HMPREF9476_00911 [Clostridium | GI: 422345924 |
| perfringens WAL-14572] | (SEQ ID NO: 365) |
| hypothetical protein RUMGNA_00563 [Ruminococcus gnavus ATCC | ZP_02039809.1 |
| 29149] >ref\|ZP_08610975.1\| NADPH-dependent butanol dehydrogenase [Lachnospiraceae | GI: 154502749 |
| bacterium 2_1_58FAA] >gb\|EDN79052.1\| NADPH-dependent butanol | (SEQ ID NO: 366) |
| dehydrogenase [Ruminococcus gnavus ATCC 29149] >gb\|EGN49760.1\| NADPH- | |
| dependent butanol dehydrogenase [Lachnospiraceae bacterium 2_1_58FAA] | |
| NADPH-dependent butanol dehydrogenase [Lachnospiraceae bacterium | ZP_08150110.1 |
| 4_1_37FAA] >gb\|EGC75414.1\| NADPH-dependent butanol dehydrogenase | GI: 325661485 |
| [Lachnospiraceae bacterium 4_1_37FAA] | (SEQ ID NO: 367) |
| iron-containing alcohol dehydrogenase [Lachnospiraceae bacterium | ZP_07957621.1 |
| 5_1_63FAA] >gb\|EFV15579.1\| iron-containing alcohol dehydrogenase [Lachnospiraceae | GI: 317499352 |
| bacterium 5_1_63FAA] | (SEQ ID NO: 368) |
| iron-containing alcohol dehydrogenase [Clostridium carboxidivorans | ZP_05393683.1 |
| P7] >ref\|ZP_06856327.1\| NADPH-dependent butanol dehydrogenase [Clostridium | GI: 255526783 |
| carboxidivorans P7] >gb\|EET85851.1\| iron-containing alcohol dehydrogenase | (SEQ ID NO: 369) |
| [Clostridium carboxidivorans P7] >gb\|EFG86503.1\| NADPH-dependent butanol | |
| dehydrogenase [Clostridium carboxidivorans P7] | |
| NADPH-dependent butanol dehydrogenase [Clostridium butyricum | ZP_02950012.1 |
| 5521] >ref\|ZP_04529712.1\| NADH-dependent butanol dehydrogenase [Clostridium | GI: 182418739 |
| butyricum E4 str. BoNTE BL5262] >gb\|EDT74989.1\| NADPH-dependent butanol | (SEQ ID NO: 370) |
| dehydrogenase [Clostridium butyricum 5521] >gb\|EEP52372.1\| NADH-dependent | |
| butanol dehydrogenase [Clostridium butyricum E4 str. BoNT E BL5262] | |
| NADPH-dependent butanol dehydrogenase [Clostridium perfringens ATCC | YP_695908.1 |
| 13124] >gb\|ABG82357.1\| NADPH-dependent butanol dehydrogenase [Clostridium | GI: 110798723 |
| perfringens ATCC 13124] | (SEQ ID NO: 371) |
| NADH-dependent butanol dehydrogenase [Clostridium botulinum A str. ATCC | YP_001254145.1 |
| 3502] >ref\|YP_001383982.1\| NADH-dependent butanol dehydrogenase [Clostridium | GI: 148379604 |
| botulinum A str. ATCC 19397] >ref\|YP_001387526.1\| NADH-dependent butanol | (SEQ ID NO: 372) |
| dehydrogenase [Clostridium botulinum A str. Hall] >ref\|YP_002804061.1\| NADH- | |
| dependent butanol dehydrogenase [Clostridium botulinum A2 str. | |
| Kyoto] >emb\|CAL83178.1\| alcohol dehydrogenase [Clostridium botulinum A str. ATCC | |
| 3502] >gb\|ABS32713.1\| NADH-dependent butanol dehydrogenase [Clostridium | |
| botulinum A str. ATCC 19397] >gb\|ABS38777.1\| NADH-dependent butanol | |
| dehydrogenase [Clostridium botulinum A str. Hall] >gb\|JACO85199.1\| NADH- | |
| dependent butanol dehydrogenase [Clostridium botulinum A2 str. Kyoto] | |
| alcohol dehydrogenase [Clostridium botulinum H04402 065] >emb\|CBZ03525.1\| | YP_005678263.1 |
| alcohol dehydrogenase [Clostridium botulinum H04402 065] | GI: 387817918 |
| | (SEQ ID NO: 373) |
| NADPH-dependent butanol dehydrogenase [Lachnospiraceae bacterium | ZP_08339921.1 |
| 2_1_46FAA] >gb\|EGG84837.1\| NADPH-dependent butanol dehydrogenase | GI: 331091079 |
| Lachnospiraceae bacterium 2_1_46FAA] | (SEQ ID NO: 374) |
| NADPH-dependent butanol dehydrogenase [Flavonifractor plautii ATCC | ZP_09382538.1 |
| 29863] >gb\|EHM54804.1\| NADPH-dependent butanol dehydrogenase [Flavonifractor | GI: 365841465 |
| plautii ATCC 29863] | (SEQ ID NO: 375) |
| hypothetical protein CLOBOL_01144 [Clostridium bolteae ATCC BAA- | ZP_02083621.1 |
| 613] >gb\|EDP18782.1\| hypothetical protein CLOBOL_01144 [Clostridium bolteae ATCC | GI: 160936248 |
| BAA-613] >gb\|ENZ38968.1\| iron-containing alcohol dehydrogenase [Clostridium | (SEQ ID NO: 376) |
| bolteae 90B8] >gb\|ENZ56854.1\| iron-containing alcohol dehydrogenase [Clostridium | |
| bolteae 90A5] >gb\|ENZ68588.1\| iron-containing alcohol dehydrogenase [Clostridium | |
| bolteae 90B7] | |
| hypothetical protein CKR_0480 [Clostridium kluyveri NBRC | YP_002470945.1 |
| 12016] >dbj\|BAH05531.1\| hypothetical protein [Clostridium kluyveri NBRC 12016] | GI: 219853823 |
| | (SEQ ID NO: 377) |
| NADPH-dependent butanol dehydrogenase [Clostridium perfringens C str. | ZP_02864967.1 |
| JGS1495] >gb\|EDS79979.1\| NADPH-dependent butanol dehydrogenase [Clostridium | GI: 169343976 |
| perfringens C str. JGS1495] | (SEQ ID NO: 378) |
| butanol dehydrogenase [Clostridium sp. G117] | AGE10401.1 |
| | GI: 444439764 |
| | (SEQ ID NO: 379) |
| NADPH-dependent butanol dehydrogenase [Clostridium colicanis 209318] | ENZ02568.1 |
| | GI: 480658532 |
| | (SEQ ID NO: 380) |
| NADH-dependent butanol dehydrogenase [Clostridium botulinum F str. | YP_001390980.1 |
| Langeland] >ref\|YP_005674617.1\| NADH-dependent butanol dehydrogenase [Clostridium | GI: 153939046 |
| botulinum F str. 230613] >gb\|ABS40440.1\| NADH-dependent butanol | (SEQ ID NO: 381) |
| dehydrogenase [Clostridium botulinum F str. Langeland] >gb\|ADF99416.1\| NADH- | |
| dependent butanol dehydrogenase [Clostridium botulinum F str. 230613] | |
| iron-containing alcohol dehydrogenase [Clostridium ljungdahlii DSM | YP_003780011.1 |
| 13528] >gb\|ADK14909.1\| predicted iron-containing alcohol dehydrogenase | GI: 300855027 |
| [Clostridium ljungdahlii DSM 13528] | (SEQ ID NO: 382) |
| iron-containing alcohol dehydrogenase [Clostridium cellulovorans | YP_003845251.1 |
| 743B] >ref\|ZP_07629738.1\| iron-containing alcohol dehydrogenase [Clostridium | GI: 302876618 |
| cellulovorans 743B] >gb\|ADL53487.1\| iron-containing alcohol dehydrogenase | (SEQ ID NO: 383) |
| [Clostridium cellulovorans 743B] | |
| NADPH-dependent butanol dehydrogenase [Lachnospiraceae bacterium | ZP_07958427.1 |
| 8_1_57FAA] >ref\|ZP_08336507.1\| NADPH-dependent butanol dehydrogenase | GI: 317500195 |
| [Lachnospiraceae bacterium 3_1_46FAA] >ref\|ZP_08617642.1\| NADPH-dependent | (SEQ ID NO: 384) |
| butanol dehydrogenase [Lachnospiraceae bacterium 1_1_57FAA] >gb\|EFV20449.1\| | |
| NADPH-dependent butanol dehydrogenase [Lachnospiraceae bacterium | |
| 8_1_57FAA] >gb\|EGG80081.1\| NADPH-dependent butanol dehydrogenase | |
| [Lachnospiraceae bacterium 3_1_46FAA] >gb\|EGN48122.1\| NADPH-dependent | |
| butanol dehydrogenase [Lachnospiraceae bacterium 1_1_57FAA] | |
| hypothetical protein C824_02214 [Clostridium sp. ASF502] | EMZ26775.1 |
| | GI: 476619921 |
| | (SEQ ID NO: 385) |
| NADH-dependent butanol dehydrogenase [Clostridium butyricum DKU-01] | EMU52057.1 |
| | GI: 475994253 |
| | (SEQ ID NO: 386) |
| NADPH-dependent butanol dehydrogenase [Clostridium botulinum B str. Eklund | YP_001887401.1 |
| 17B] >gb\|ACD23887.1\| NADH-dependent butanol dehydrogenase [Clostridium | GI: 187934513 |
| botulinum B str. Eklund 17B] | (SEQ ID NO: 387) |
| NADH-dependent butanol dehydrogenase, partial [Clostridium botulinum | ZP_16269747.1 |
| CFSAN001627] >gb\|EKN42565.1\| NADH-dependent butanol dehydrogenase, partial | GI: 421834809 |
| [Clostridium botulinum CFSAN001627] | (SEQ ID NO: 388) |
| Alcohol dehydrogenase, class IV [Ruminococcus torques L2-14] >emb\|CBL26689.1\| | YP_007787197.1 |
| Alcohol dehydrogenase, class IV [Ruminococcus torques L2-14] | GI: 479157602 |
| | (SEQ ID NO: 389) |
| iron-containing alcohol dehydrogenase [Clostridium carboxidivorans | ZP_05391085.1 |
| P7] >gb\|EET88390.1\| iron-containing alcohol dehydrogenase [Clostridium | GI: 255524125 |
| carboxidivorans P7] >gb\|ADO12118.1\| NADH-dependent butanol dehydrogenase | (SEQ ID NO: 390) |
| [Clostridium carboxidivorans P7] | |
| alcohol dehydrogenase [Clostridium beijerinckii] | AAM18709.1 |
| | GI: 20279078 |
| | (SEQ ID NO: 391) |
| iron-containing alcohol dehydrogenase [Clostridium carboxidivorans | ZP_05392917.1 |
| P7] >gb\|EET86647.1\| iron-containing alcohol dehydrogenase [Clostridium | GI: 255525992 |
| carboxidivorans P7] | (SEQ ID NO: 392) |
| Alcohol dehydrogenase [Clostridium sp. DL-VIII] >gb\|EHI97343.1\| Alcohol | ZP_09202750.1 |
| dehydrogenase [Clostridium sp. DL-VIII] | GI: 359410285 |
| | (SEQ ID NO: 393) |
| hypothetical protein RUMTOR_02695 [Ruminococcus torques ATCC | ZP_01969110.1 |
| 27756] >gb\|EDK23148.1\| NADPH-dependent butanol dehydrogenase [Ruminococcus | GI: 153816442 |
| torques ATCC 27756] | (SEQ ID NO: 394 |
| iron-containing alcohol dehydrogenase [Clostridium saccharolyticum | YP_003821060.1 |
| WM1] >gb\|ADL03437.1\| iron-containing alcohol dehydrogenase [Clostridium | GI: 302385238 |
| saccharolyticum WM1] | (SEQ ID NO: 395) |
| NADPH-dependent butanol dehydrogenase [Clostridium ljungdahlii DSM | YP_003782121.1 |
| 13528] >gb\|ADK17019.1\| predicted NADPH-dependent butanol dehydrogenase | GI: 300857137 |
| [Clostridium ljungdahlii DSM 13528] | (SEQ ID NO: 396) |
| NADPH-dependent butanol dehydrogenase [Clostridium butyricum 60E.3] | ENZ30148.1 |
| | GI: 480687146 |
| | (SEQ ID NO: 397) |
| NADPH-dependent butanol dehydrogenase [Clostridium perfringens D str. | ZP_02952006.1. |
| JGS1721] >gb\|EDT73002.1\| NADPH-dependent butanol dehydrogenase [Clostridium | GI: 182624220 |
| perfringens D str. JGS1721] | (SEQ ID NO: 398) |
| NADPH-dependent butanol dehydrogenase [Clostridium perfringens CPE str. | ZP_02638669.1 |
| F4969] >gb\|EDT27639.1\| NADPH-dependent butanol dehydrogenase [Clostridium | GI: 168213044 |
| perfringens CPE str. F4969] | (SEQ ID NO: 399) |
| iron-containing alcohol dehydrogenase [Clostridium beijerinckii NCIMB | YP_001309304.1 |
| 8052] >gb\|AAT38119.1\| alcohol dehydrogenase [Clostridium beijerinckii NCIMB | GI: 150017050 |
| 8052] >gb\|ABR34348.1\| iron-containing alcohol dehydrogenase [Clostridium | (SEQ ID NO: 400) |
| beijerinckii NCIMB 8052] | |
| NADPH-dependent butanol dehydrogenase Adh [Clostridium | YP_007453510.1 |
| saccharoperbutylacetonicum N1-4(HMT)] >gb\|AGF54256.1\| NADPH-dependent | GI: 451817309 |
| butanol dehydrogenase Adh [Clostridium saccharoperbutylacetonicum N1-4(HMT)] | (SEQ ID NO: 401) |
| hypothetical protein BACCAP_02245 [Bacteroides capillosus ATCC | ZP_02036635.1 |
| 29799] >gb\|EDM99972.1\| NADPH-dependent butanol dehydrogenase | GI: 154498257 |
| [Pseudoflavonifractor capillosus ATCC 29799] | (SEQ ID NO: 402) |
| hypothetical protein RUMLAC_02095 [Ruminococcus lactaris ATCC | ZP_03168412.1 |
| 29176] >gbEDY31932.1\| NADPH-dependent butanol dehydrogenase [Ruminococcus | GI: 197303373 |
| lactaris ATCC 29176] | (SEQ ID NO: 403) |
| NADPH-dependent butanol dehydrogenase [Clostridium clostridioforme | ZP_09116768.1 |
| 2_1_49FAA] >gb\|EHG29197.1\| NADPH-dependent butanol dehydrogenase | GI: 357055705 |
| [Clostridium clostridioforme 2_1_49FAA] >gb\|ENY86875.1\| iron-containing alcohol | (SEQ ID NO: 404) |
| dehydrogenase [Clostridium clostridioforme CM201] >gb\|ENZ01519.1\| iron- | |
| containing alcohol dehydrogenase [Clostridium clostridioforme | |
| 90B1] >gb\|ENZ13590.1\| iron-containing alcohol dehydrogenase [Clostridium | |
| clostridioforme 90A8] >gb\|ENZ21103.1\| iron-containing alcohol dehydrogenase | |
| [Clostridium clostridioforme 90A3] >gb\|ENZ23413.1\| iron-containing alcohol | |
| dehydrogenase [Clostridium clostridioforme 90A1] >gb\|ENZ59818.1\| iron-containing | |
| alcohol dehydrogenase [Clostridium clostridioforme 90A6] >gb\|ENZ65377.1\| iron- | |
| containing alcohol dehydrogenase [Clostridium clostridioforme 90A4] | |
| hypothetical protein CLONEX_01601 [Clostridium nexile DSM | ZP_03289399.1 |
| 1787] >gb\|EEA82540.1\| hypothetical protein CLONEX_01601 [Clostridium nexile DSM | GI: 210612608 |
| 1787] | (SEQ ID NO: 405) |
| NADPH-dependent butanol dehydrogenase [Clostridium tetani | NP_782951.1 |
| E88] >gb\|AAO36888.1\| NADPH-dependent butanol dehydrogenase [Clostridium tetani | GI: 28212007 |
| E88] | (SEQ ID NO: 406) |
| NADH-dependent butanol dehydrogenase [Clostridium sporogenes PA | ZP_18252471.1 |
| 3679] >gb\|EHN16013.1\| NADH-dependent butanol dehydrogenase [Clostridium | GI: 424827700 |
| sporogenes PA 3679] | (SEQ ID NO: 407) |
| iron-containing alcohol dehydrogenase [Clostridium beijerinckii NCIMB | YP_001308850.1 |
| 8052] >gb\|ABR33894.1\| iron-containing alcohol dehydrogenase [Clostridium | GI: 150016596 |
| beijerinckii NCIMB 8052] | (SEQ ID NO: 408) |
| NADPH-dependent butanol dehydrogenase [Clostridium botulinum E3 str. Alaska | YP_001922335.1 |
| E43] >gb\|ACD51034.1\| NADH-dependent butanol dehydrogenase [Clostridium | GI: 150016596 |
| botulinum E3 str. Alaska E43] | (SEQ ID NO: 409) |
| NADH-dependent butanol dehydrogenase [Clostridium botulinum B1 str. | YP_001781276.1 |
| Okra] >ref\|ZP_19210096.1\| NADH-dependent butanol dehydrogenase [Clostridium | GI: 170757418 |
| botulinum CFSAN001628] >gb\|ACA46466.1\| NADH-dependent butanol | (SEQ ID NO: 410) |
| dehydrogenase [Clostridium botulinum B1 str. Okra] >gb\|EKX78737.1\| NADH- | |
| dependent butanol dehydrogenase [Clostridium botulinum CFSAN001628] | |
| Alcohol dehydrogenase, class IV [butyrate-producing bacterium | YP_007825178.1 |
| SS3/4] >emb\|CBL42701.1\| Alcohol dehydrogenase, class IV [butyrate-producing | GI: 479195640 |
| bacterium SS3/4] | (SEQ ID NO: 411) |
| hypothetical protein ANACAC_01842 [Anaerostipes caccae DSM | ZP_02419256.1 |
| 14662] >gb\|EDR98218.1\| NADPH-dependent butanol dehydrogenase [Anaerostipes | GI: 167747129 |
| caccae DSM 14662] | (SEQ ID NO: 412) |
| NADPH-dependent butanol dehydrogenase [Clostridium sp. | ZP_06346636.2 |
| M62/1] >gb\|EFE12215.1\| NADPH-dependent butanol dehydrogenase [Clostridium sp. | GI: 291087504 |
| M62/1] | (SEQ ID NO: 413) |
| Alcohol dehydrogenase, class IV [Clostridium cf. saccharolyticum | YP_007848113.1 |
| K10] >emb\|CBK76115.1\| Alcohol dehydrogenase, class IV [Clostridium cf. | GI: 479336895 |
| saccharolyticum K10] | (SEQ ID NO: 414) |
| hypothetical protein CLOSTASPAR_04682 [Clostridium asparagiforme DSM | ZP_03760651.1 |
| 15981] >gb\|EEG53230.1\| hypothetical protein CLOSTASPAR_04682 [Clostridium | GI: 225405462 |
| asparagiforme DSM 15981] | (SEQ ID NO: 415) |
| alcohol dehydrogenase [Clostridium beijerinckii] | AAM18705.1 |
| | GI: 20279073 |
| | (SEQ ID NO: 416) |
| NADPH-dependent butanol dehydrogenase [Clostridium sp. D5] >gb\|EGB92190.1\| | ZP_08130603.1 |
| NADPH-dependent butanol dehydrogenase [Clostridium sp. D5] | GI: 325263870 |
| | (SEQ ID NO: 417) |
| iron-containing alcohol dehydrogenase [Butyrivibrio proteoclasticus | YP_003830744.1 |
| B316] >gb\|ADL34162.1\| iron-containing alcohol dehydrogenase [Butyrivibrio | GI: 302670784 |
| proteoclasticus B316] | (SEQ ID NO: 418) |
| iron-containing alcohol dehydrogenase [Clostridium hathewayi 12489931] | ENY95324.1 |
| | GI: 480651035 |
| | (SEQ ID NO: 419) |
| NADPH-dependent butanol dehydrogenase [Dorea formicigenerans | ZP_08848505.1 |
| 4_6_53AFAA] >gb\|EGX69980.1\| NADPH-dependent butanol dehydrogenase [Dorea | GI: 346306347 |
| formicigenerans 4_6_53AFAA] | (SEQ ID NO: 420) |
| NADPH-dependent butanol dehydrogenase [Clostridium sp. | ZP_10977308.1 |
| 7_2_43FAA] >gb\|EEH97058.1\| NADPH-dependent butanol dehydrogenase [Clostridium sp. | GI: 404372007 |
| 7_2_43FAA] | (SEQ ID NO: 421) |
| hypothetical protein HMPREF1006_02359 [Synergistes sp. | ZP_09360726.1 |
| 3_1_syn1] >gb\|EHL70408.1\| hypothetical protein HMPREF1006_02359 [Synergistes sp. | GI: 365167520 |
| 3_1_syn1] | (SEQ ID NO: 422) |
| NADH-dependent butanol dehydrogenase [Clostridium butyricum DKU-01] | EMU52053.1 |
| 100% | GI: 475994249 |
| | (SEQ ID NO: 423) |
| NADPH-dependent butanol dehydrogenase [Lachnospiraceae bacterium | ZP_08334039.1 |
| 9_1_43BFAA] >gb\|EGG88117.1\| NADPH-dependent butanol dehydrogenase | GI: 331084952 |
| [Lachnospiraceae bacterium 9_1_43BFAA] | (SEQ ID NO: 424) |
| NADPH-dependent butanol dehydrogenase [Clostridium butyricum 60E.3] | ENZ30152.1 |
| | GI: 480687150 |
| | (SEQ ID NO: 425) |
| NADPH-dependet butanol dehydrogenase [Clostridium saccharobutylicum] | CAQ53139.1 |
| | GI: 188027005 |
| | (SEQ ID NO: 426) |
| NADPH-dependent butanol dehydrogenase [Clostridium perfringens str. | NP_562172.1 |
| 13] >dbj\|BAB80962.1\| NADPH-dependent butanol dehydrogenase [Clostridium | GI: 18310238 |
| perfringens str. 13] | (SEQ ID NO: 427) |
| NADPH-dependent butanol dehydrogenase [Clostridium botulinum D str. | ZP_04861753.1 |
| 1873] >gb\|EES92245.1\| NADPH-dependent butanol dehydrogenase [Clostridium | GI: 253680950 |
| botulinum D str. 1873] | (SEQ ID NO: 428) |
| alcohol dehydrogenase [Clostridium lentocellum DSM 5427] >gb\|ADZ84386.1\| | YP_004309584.1 |
| Alcohol dehydrogenase [Clostridium lentocellum DSM 5427] | GI: 326791763 |
| | (SEQ ID NO: 429) |
| NADPH-dependent butanol dehydrogenase [Clostridium botulinum C str. | ZP_02620674.1 |
| Eklund] >gb\|EDS78083.1\| NADPH-dependent butanol dehydrogenase [Clostridium | GI: 168186039 |
| botulinum C str. Eklund] | (SEQ ID NO: 430) |
| NADPH-dependent butanol dehydrogenase [Clostridium perfringens | ZP_16920614.1 |
| F262] >gb\|EIA17164.1\| NADPH-dependent butanol dehydrogenase [Clostridium | GI: 422874129 |
| perfringens F262] | (SEQ ID NO: 431) |
| hypothetical protein CLOSS21_02605 [Clostridium sp. SS2/1] >gb\|EDS20969.1\| | ZP_02440114.1 |
| NADPH-dependent butanol dehydrogenase [Clostridium sp. SS2/1] | GI: 167768061 |
| | (SEQ ID NO: 432) |
| alcohol dehydrogenase, class IV [Clostridium sp. Maddingley | ZP_11366418.1 |
| MBC34-26] >gb\|EKQ51809.1\| alcohol dehydrogenase, class IV [Clostridium sp. | GI: 410728237 |
| Maddingley MBC34-26] | (SEQ ID NO: 433) |
| NADPH-dependent butanol dehydrogenase [Lachnospiraceae bacterium | ZP_08614635.1 |
| 1_4_56FAA] >gb\|EGN32435.1\| NADPH-dependent butanol dehydrogenase | GI: 336434916 |
| [Lachnospiraceae bacterium 1_4_56FAA] | (SEQ ID NO: 434) |
| iron-containing alcohol dehydrogenase [Clostridium botulinum | YP_004396877.1 |
| BKT015925] >gb\|AEB76880.1\| iron-containing alcohol dehydrogenase [Clostridium | GI: 331270385 |
| botulinum BKT015925] | (SEQ ID NO: 435) |
| iron-containing alcohol dehydrogenase [Clostridium clostridioforme | ENZ14597.1 |
| 90A7] >gb\|ENZ44225.1\| iron-containing alcohol dehydrogenase [Clostridium bolteae | GI: 480671083 |
| 90B3] >gb\|ENZ49956.1\| iron-containing alcohol dehydrogenase [Clostridium bolteae | (SEQ ID NO: 436) |
| 90A9] | |
| hypothetical protein DORLON_00585 [Dorea longicatena DSM | ZP_01994600.1 |
| 13814] >gb\|EDM63908.1\| NADPH-dependent butanol dehydrogenase [Dorea longicatena | GI: 153853191 |
| DSM 13814] | (SEQ ID NO: 437) |
| iron-containing alcohol dehydrogenase [Anaerostipes sp. | ZP_07932949.1 |
| 3_2_56FAA] >gb\|EFV20975.1\| iron-containing alcohol dehydrogenase [Anaerostipes | GI: 317473660 |
| sp. 3_2_56FAA] | (SEQ ID NO: 438) |

Table 56 provides exemplary ADH enzyme variants. Table 56 shows exemplary enzyme variant positions with the corresponding amino acid for a particular position that were identified as providing a desirable activity, including ADH activity, BDO production, and the like. It is understood that the individual ADH variants such as those described in Table 56 can be used alone, or can be combined with any other variant amino acid position, including 2, 3, 4, 5, 6, 7, 8, 9, 10, greater than 10 and up to all variant amino acid positions as disclosed herein, to generate additional variants having desirable activities.

TABLE 56
Exemplary ADH enzyme variants.

| | | Corresponding Positions | Exemplary Enzyme Sequence Indicating |
|---|---|---|---|
| Wild-Type Designation | Wild-Type Sequence | and Amino Acid Changes | Corresponding Positions |
| bifunctional acetaldehyde- | 1 MKVTNQKELK QKLNELREAQ KKFATYTQEQ | 34H, 35A, 487K | 1 MKVTNQKELK QKLNELREAQ KKFATYTQEQ |
| CoA/alcohol | VDKIFKQCAI AAAKERINLA KLAVEETGIG | (alternative; not shown), | VDKIFKQCAI AAAKERINLA KLAVEETGIG |
| dehydrogenase | 61 LVEDKIIKNH FAAEYIYNKY KNEKTCGIID | 619G, 648V, 648C | 61 LVEDKIIKNH FAAEYIYNKY KNEKTCGIID |
| [Clostridium | HDDSLGITKV AEPIGIVAAI VPTTNPTSTA | (alternative; not shown), | HDDSLGITKV AEPIGIVAAI VPTTNPTSTA |
| acetobutylicum ATCC 824] | 121 IFKSLISLKT RNAIFFSPHP RAKKSTIAAA | 653F, 659G (alternative; | 121 IFKSLISLKT RNAIFFSPHP RAKKSTIAAA |
| Sequence ID: | KLILDAAVKA GAPKNIIGWI DEPSIELSQD | not shown), 659T, 726Q, | KLILDAAVKA GAPKNIIGWI DEPSIELSQD |
| ref\|NP_149199.1\| | 181 LMSEADIILA TGGPSMVKAA YSSGKPAIGV | 726V (alternative; not | 181 LMSEADIILA TGGPSMVKAA YSSGKPAIGV |
| Length: 858 | GAGNTPAIID ESADIDMAVS SIILSKTYDN | shown), 726L | GAGNTPAIID ESADIDMAVS SIILSKTYDN |
| | 241 GVICASEQSI LVMNSIYEKV KEEFVKRGSY | (alternative; not shown), | 241 GVICASEQSI LVMNSIYEKV KEEFVKRGSY |
| | ILNQNEIAKI KETMFKNGAI NADIVGKSAY | 726S (alternative; not | ILNQNEIAKI KETMFKNGAI NADIVGKSAY |
| | 301 IIAKMAGIEV PQTTKILIGE VQSVEKSELF | shown), 729P, 277T, | 301 IIAKMAGIEV PQTTKILIGE VQSVEKSELF |
| | SHEKLSPVLA MYKVKDFDEA LKKAQRLIEL | 837S, 206R, 222H, 127H, | SHEKLSPVLA MYKVKDFDEA LKKAQRLIEL |
| | 361 GGSGHTSSLY IDSQNNKDKV KEFGLAMKTS | 592D, 334L, | 361 GGSGHTSSLY IDSQNNKDKV KEFGLAMKTS |
| | RTFINMPSSQ GASGDLYNFA IAPSFTLGCG | | RTFINMPSSQ GASGDLYNFA IAPSFTLGCG |
| | 421 TWGGNSVSQN VEPKHLLNIK SVAERRENML | | 421 TWGGNSVSQN VEPKHLLNIK |
| | WFKVPQKIYF KYGCLRFALK ELKDMNKKRA | | SVAERRENML WFKVPQKIYF KYGCLRFALK |
| | 481 FIVTDKDLFK LGYVNKITKV LDEIDIKYSI | | ELKDMNKKRA |
| | FTDIKSDPTI DSVKKGAKEM LNFEPDTIIS | | 481 FIVTDKDLFK LGYVNKITKV LDEIDIKYSI |
| | 541 IGGGSPMDAA KVMHLLYEYP EAEIENLAIN | | FTDIKSDPTI DSVKKGAKEM LNFEPDTIIS |
| | FMDIRKRICN FPKLGTKAIS VAIPTTAGTG | | 541 IGGGSPMDAA KVMHLLYEYP EAEIENLAIN |
| | 601 SEATPFAVIT NDETGMKYPL TSYELTPNMA | | FMDIRKRICN FPKLGTKAIS VDIPTTAGTG |
| | IIDTELMLNM PRKLTAATGI DALVHAIEAY | | 601 SEATPFAVIT NDETGMKYGL TSYELTPNMA |
| | 661 VSVMATDYTD ELALRAIKMI FKYLPRAYKN | | IIDTELMLNM PRKLTAAVGI DAFVHAIETY |
| | GTNDIEAREK MAHASNIAGM AFANAFLGVC | | 661 VSVMATDYTD ELALRAIKMI FKYLPRAYKN |
| | 721 HSMAHKLGAM HHVPHGIACA VLIEEVIKYN | | GTNDIEAREK MAHASNIAGM AFANAFLGVC |
| | ATDCPTKQTA FPQYKSPNAK RKYAEIAEYL | | 721 HSMAHQLGPM HHVPHGIACA |
| | 781 NLKGTSDTEK VTALIEAISK LKIDLSIPQN | | VLIEEVIKYN ATDCPTKQTA FPQYKSPNAK |
| | ISAAGINKKD FYNTLDKMSE LAFDDQCTTA | | RKYAEIAEYL |
| | 841 NPRYPLISEL KDIYIKSF (SEQ ID NO: 210) | | 781 NLKGTSDTEK VTALIEAISK LKIDLSIPQN |
| | | | ISAAGINKKD FYNTLDKMSE LAFDDQSTTA |
| | | | 841 NPRYPLISEL KDIYIKSF (SEQ ID NO: 222) |
| NADPH-dependent | 1 MKRFTIPRDI YYGKGSLEVI KSIKGNKAVI | 34H, 35A, 35K | 1 MKRFTIPRDI YYGKGSLEVI KSIKGNKAVI |
| butanol dehydrogenase | VIGGGSMKRF GFLDKVENYL KEAGIETKLI | (alternative; not shown), | VIGHASMKRF GELDKVENYL KEAGIETKLI |
| [Clostridium tetani E88] | 61 EGVEPDPSVE TVMKGAEIMR EFKPDWIISI | 161G, 190V, 190C | 61 EGVEPDPSVE TVMKGAEIMR EFKPDWIISI |
| Sequence ID: | GGGSPIDAAK AMWLFYEHPD LTFEEAAQKV | (alternative; not shown), | GGGSPIDAAK AMWLFYEHPD LTFEEAAQKV |
| ref\|NP_782951.1\| | 121 LCLPELRSKA KFMAIPSTSG TASEVTAFSV | 195F, 201G (alternative; | 121 LCLPELRHKA KFMDIPSTSG TASEVTAFSV |
| Length: 389 | ITDYKKQIKY PLADFNLTPD IAIVDPELAE | not shown), 201T, 265Q, | ITDYKKQIKY GLADFNLTPD IAIVDPELAE |
| | 181 TMPAKLTAHT GMDALTHAIE AYVATENTSF | 265V (alternative; not | 181 TMPAKLTAHV GMDALTHAIE TYVATERTSF |
| | TDPLALQAIL MVKDYLIKSY EEDKEARERM | shown), 265L | TDPLALQAIL MVHDYLIKSY EEDKEARERM |
| | 241 HEAQCLAGMA FSNALLGITH SMAHKIGAVF | (alternative; not shown), | 241 HEAQCLAGMA FSNALLGITH |
| | DITHGCANAI FLPYVIQFNS KVCEERYANI | 265S (alternative; not | SMAHQIGPVF DITHGCATAI FLPYVIQFNS |
| | 301 AKYLGLEGES DKDLVNSLIN FIRDLNKKLS | shown), 268P, 278T, | KVCEERYANI |
| | IDNSLKEYGI DENEFKEKVE YMAHNAVLDA | 361S, 207R, 223H, 128H, | 301 AKYLGLEGES DKDLVNSLIN FIRDLNKKLS |
| | 361 CTGANPRKIN EEEMKNMYTY AFNGKDIDF | 134D, 335L, | IDNSLKEYGI DENEFKEKVE YMAHNAVLDA |
| | (SEQ ID NO: 211) | | 361 CTGANPRKIN EEEMKNMYTY AFNGKDIDF |
| | | | (SEQ ID NO: 223) |
| iron-containing alcohol | 1 MSRFTLPRDI YHGKGALEAL KTLEGKRAIV | 34H, 35A, | 1 MSRFTLPRDI YHGKGALEAL KTLEGKRAIV |
| dehydrogenase | CVGGSSMKKG GFLQKVEDYL KEAGMEVELF | 35K (alternative; not | CVGHASMKKG GFLQKVEDYL KEAGMEVELF |
| [Butyrivibrio | 61 EGIEPDPSVE TVMKGAEAMQ KFQPDWIVAI | shown), 160G, 189V, | 61 EGIEPDPSVE TVMKGAEAMQ KFQPDWIVAI |
| proteoclasticus B316] | GGGSPIDAAK AMWIKYEYPE TTFEDMCKVF | 189C (alternative; not | GGGSPIDAAK AMWIKYEYPE TTFEDMCKVF |
| Sequence ID: | 121 GLPKLRTKAH FCAIPSTSGT ATEVTAFSII | shown), 194F, 200G | 121 GLPKLRHKAH FCDIPSTSGT ATEVTAFSII |
| ref\|YP_003830744.1\| | TDYQKGIKYP IADFEITPDV AIVDPELTHT | (alternative; not shown), | TDYQKGIKYG IADFEITPDV AIVDPELTHT |
| Length: 405 | 181 MPIKLVAHTG MDAMTHAIEA YVSTANCDYT | 200T, 264Q, 264V | 181 MPIKLVAHVG MDAFTHAIET YVSTARCDYT |
| | DGLAIHAIEM IQANLVKSYN GDMESRDAMH | (alternative; not shown), | DGLAIHAIEM IHANLVKSYN GDMESRDAMH |
| | 241 NAQCLAGMAF SNALLGIVHS | 264L (alternative; not | 241 NAQCLAGMAF SNALLGIVHS |
| | MAHKTGAVFA DKGAHIIHGA ANAMYLPKVI | shown), 264S | MAHQTGPVFA DKGAHIIHGA ATAMYLPKVI |
| | AFNAKDETAK | (alternative; not shown), | AFNAKDETAK |
| | 301 KRYGVIADYM HLGGSNDDEK VKLLIDYLRK | 267P, 282T, 377S, 206R, | 301 KRYGVIADYM HLGGSNDDEK VKLLIDYLRK |
| | MNDDLNIPHS INHYGADGLP ADQGFVAEDV | 222H, 127H, 133D, 334L, | MNDDLNIPHS LNHYGADGLP ADQGFVAEDV |
| | 361 FLERLHDIAA NAILDACTGS NPRQPSQEEM | | 361 FLERLHDIAA NAILDACTGS NPRQPSQEEM |
| | EKLLKCCYYD TEVDF (SEQ ID NO: 212) | | EKLLKCCYYD TEVDF (SEQ ID NO: 224) |
| hypothetical protein | 1 mgrftlprdl yfgkgsletl ktldgkkavl | 34H, 35A, | 1 MGRFTLPRDL YFGKGSLETL KTLDGKKAVL |
| ANACAC_01842 | vlgggsmkrf gfvdqvvdyl keagietkli | 35K (alternative; not | VLGHASMKRF GFVDQVVDYL KEAGIETKLI |
| [Anaerostipes caccae DSM | 61 egvepdpsve tvfkgaevmr dfepdwiiam | shown), 160G, 189V, | 61 EGVEPDPSVE TVFKGAEVMR DFEPDWIIAM |
| 14662] | gggspidaak amwifyehpe ttfddvkdpf | 189C (alternative; not | GGGSPIDAAK AMWIFYEHPE TTFDDVKDPF |
| Sequence ID: | 121 tvpplrnkak flaipstsgt atevtafavi | shown), 194F, 200G | 121 TVPPLRHKAK FLDIPSTSGT ATEVTAFAVI |
| ref\|ZP_02419256.1\| | tdyssgikyp ladfeitpdi aivdpalaet | (alternative; not shown), | TDYSSGIKYG LADFEITPDI AIVDPALAET |
| | 181 mppkltsytg mdalthaiea yvaglhspfs | 200T, 264Q, 264V | 181 MPPKLTSYVG MDAFTHAIET YVAGLRSPFS |
| | dplameaiem vfdnlkasye gdmtarenmh | (alternative; not shown), | DPLAMEAIEM VHDNLKASYE GDMTARENMH |
| | 241 yaqclagmsf snallgivhs lahktgavfd | 264L (alternative; not | 241 YAQCLAGMSF SNALLGIVHS LAHQTGPVFD |
| | tghithgcan aiylpyviqf nakvceerya | shown), 264S | TGHITHGCAT AIYLPYVIQF NAKVCEERYA |
| | 301 aiarriglpg nsdkaltlsl ieairemnkk | (alternative; not shown), | 301 AIARRIGLPG NSDKALTLSL IEAIREMNKK |
| | lnivsnlkdf gineeefnqk krmiaerava | 267P, 280T, 363S, 206R, | LNIVSNLKDF GINEEEFNQK KRMIAERAVA |
| | 361 dactgsnpre tsvddmekvl eciyygkdvt f | 222H, 127H, A133D, 337L | 361 DASTGSNPRE TSVDDMEKVL ECIYYGKDVT |
| | (SEQ ID NO: 213) | | F (SEQ ID NO: 225) |
| NADPH-dependent | 1 MARFTLPRDL YWGKGSLENL KTLQGKKAVL | 34H, 35A, 35K | 1 MARFTLPRDL YWGKGSLENL KTLQGKKAVL |
| butanol dehydrogenase | VLGGGSMKRF GFVDKAVSYL KEAGIETRLF | (alternative; not shown), | VLGHASMKRF GFVDKAVSYL KEAGIETRLF |
| [Lachnospiraceae | 61 ENVEPDPSVE TVMKGAEFMR EFEPDWIIAM | 160G, 189V, 189C | 61 ENVEPDPSVE TVMKGAEFMR |
| bacterium 6_1_63FAA] | GGGSPIDAAK AMWVFYEYPE CTFEEILTPF | (alternative; not shown), | EFEPDWIIAM GGGSPIDAAK AMWVFYEYPE |
| NCBI Reference Sequence: | 121 SFPELRKKAK FCAIPSTSGT ATEVTAFSVI | 194F, 200G (alternative; | CTFEEILTPF |
| ZP_08331580.1 | TDYAKGIKYP LADFNITPDV AIVDPELAET | not shown), 200T, 264Q, | 121 SFPELRHKAK FCDIPSTSGT ATEVTAFSVI |
| | 181 MPKHLVAYTG MDALTHAIEA YVSTLHGTFT | 264V (alternative; not | TDYAKGIKYG LADFNITPDV AIVDPELAET |
| | DPLALKAIQI VNDELTKSYD GDMASREEMH | shown), 264L | 181 MPKHLVAYVG MDALTHAIET YVSTLRGTFT |
| | 241 YGQCLAGMAF SNALLGIVHS MAHKTGAAFS | (alternative; not shown), | DPLALKAIQI VHDELTKSYD GDMASREEMH |
| | TGHITHGLAN AMYLPYVIKY NAKEAEAKKR | 264S (alternative; not | 241 YGQCLAGMAF SNALLGIVHS |
| | 301 YETIAAYLGL EASVDALCNR IVELNNYMGI | shown), 267P, 280T, | MAHQTGPAFS TGHITHGLAN AMYLPYVIKY |
| | PNTLKEFGIK EDEFKEKISE IAKNAVGDAC | 360S, 206R, 222H, 127H, | NAKEAEAKKR |
| | 361 TGSNPRSIDP ETMEKLFTYI YDGKEVDF (SEQ | 133D, 334L | 301 YETIAAYLGL EASVDALCNR IVELNNYMGI |
| | ID NO: 214) | | PNTLKEFGIK EDEFKEKISE IAKNAVGDAS |
| | | | 361 TGSNPRSIDP ETMEKLFTYI YDGKEVDF |
| | | | (SEQ ID NO: 226) |
| NADPH-dependent | 1 MARFTLPRDI YHGNGSLEVL KTLKGKKAVV | 34H, 35A, 35K | 1 MARFTLPRDI YHGNGSLEVL KTLKGKKAVV |
| butanol dehydrogenase | VVGGGSMKRF GFLEKVENYL KEAGLEVRLI | (alternative; not shown), | VVGHASMKRF GFLEKVENYL KEAGLEVRLI |
| [Clostridium celatum DSM | 61 EGVEPDPSVE TVMNGAAVMR | 160G, 189V, 189C | 61 EGVEPDPSVE TVMNGAAVMR |
| 1785] | EFEPDWIVAM GGGSPIDAAK AMWIFYEYPD | (alternative; not shown), | EFEPDWIVAM GGGSPIDAAK AMWIFYEYPD |
| Sequence ID: | FTFEEAVVPF | 194F, 200G (alternative; | FTFEEAVVPF |
| ref\|ZP_19299687.1\| | 121 GLPELRQKAK FVAIPSTSGT ATEVTAFSVI | not shown), 200T, 264Q, | 121 GLPELRHKAK FVDIPSTSGT ATEVTAFSVI |
| Length: 388 | TDYKAKIKYP LADFNITPDI AIVDPELAQT | 264V (alternative; not | TDYKAKIKYG LADFNITPDI AIVDPELAQT |
| | 181 MPAKLVAHTG MDALTHAIEA YTASLKSNFS | shown), 264L | 181 MPAKLVAHVG MDAFTHAIET YTASLRSNFS |
| | DPLALKAIEM INENLVKSFN GDKNARDLMH | (alternative; not shown), | DPLALKAIEM IHENLVKSFN GDKNARDLMH |
| | 241 EAQCLAGMAF SNALLGIVHS MAHKTGAVFH | 264S (alternative; not | 241 EAQCLAGMAF SNALLGIVHS |
| | IPHGCANAIF LPYVIQYNRS ECEERYADIA | shown), 267P, 277T, | MAHQTGPVFH IPHGCATAIF LPYVIQYNRS |
| | 301 KVLKLEGNTN AELTDSLITK INEFNKELNI | 360S, 206R, 222H, 127H, | ECEERYADIA |
| | PATMQEYGVN EEEFLANLKN VAHNAVLDAC | 133D, 334L, | 301 KVLKLEGNTN AELTDSLITK INEFNKELNI |
| | 361 TGSNPRTIDD ETMEKLLSCT FYGNKVDF | | PATLQEYGVN EEEFLANLKN VAHNAVLDAS |
| | (SEQ ID NO: 215) | | 361 TGSNPRTIDD ETMEKLLSCT FYGNKVDF |
| | | | (SEQ ID NO: 227) |
| iron-containing alcohol | 1 MSRFTLPRDI YFGENTLETL KTLKGKKAII | 34H, 35A, 35K | 1 MSRFTLPRDI YFGENTLETL KTLKGKKAII |
| dehydrogenase | VVGGGSMKKF GFLQKVEEYL KEAGMEIKLI | (alternative; not shown), | VVGHASMKKF GFLQKVEEYL KEAGMEIKLI |
| [Clostridium | 61 EGVEPDPSVE TVMKGAEIMR DFEPDWIVSI | 160G, 189V, 189C | 61 EGVEPDPSVE TVMKGAEIMR DFEPDWIVSI |
| carboxidivorans P7] | GGGSPIDAAK AMWIFYEYPE FTFEQAVVPF | (alternative; not shown), | GGGSPIDAAK AMWIFYEYPE FTFEQAVVPF |
| NCBI Reference Sequence: | 121 GIPDLRQKAK FVAIPSTSGT ATEVTAFSVI | 194F, 200G (alternative; | 121 GIPDLRHKAK FVDIPSTSGT ATEVTAFSVI |
| ZP_05391085.1 | TDYKAKIKYP LADFNLTPDV AIIDPALAQT | not shown), 200T, 264Q, | TDYKAKIKYG LADFNLTPDV AIIDPALAQT |
| | 181 MPAKLTAHTG MDALTHAIEA YVAGLRSYFS | 264V (alternative; not | 181 MPAKLTAHVG MDAFTHAIET YVAGLRSYFS |
| | DPLAMQAIVM TKDNLIKSYE GDKEARDEMH | shown), 264L | DPLAMQAIVM THDNLIKSYE GDKEARDEMH |
| | 241 IAQCLAGMAF SNALLGITHS MAHKTGAVFH | (alternative; not shown), | 241 IAQCLAGMAF SNALLGITHS |
| | IPHGCANAIF LPYVIDFNKK TCKDRYATIA | 264S (alternative; not | MAHQTGPVFH IPHGCATAIF LPYVIDENKK |
| | 301 KTLGLAGNTD DELVDALTSM IQEMNKKMDI | shown), 267P, 277T, | TCKDRYATIA |
| | PLNLKEYGVT EEDFNENLDF IAHNAVLDAC | 360S, 206R, 222H, 127H, | 301 KTLGLAGNTD DELVDALTSM |
| | 361 TGSNPRPITE EEMKKVFKCT FTGEKVNF | 133D, 334L, | IQEMNKKMDI PLNLKEYGVT EEDFNENLDF |
| | (SEQ ID NO: 216) | | IAHNAVLDAS |
| | | | 361 TGSNPRPITE EEMKKVFKCT FTGEKVNF |
| | | | (SEQ ID NO: 228) |
| NADPH-dependent | 1 MARFTLPRDI YHGEGSLEVL KTLKGKKAFV | 34H, 35A, | 1 MARFTLPRDI YHGEGSLEVL KTLKGKKAFV |
| butanol dehydrogenase | VVGGGSMKRF GFLQKVENYL KEAGMEVELF | 35K (alternative; not | VVGHASMKRF GFLQKVENYL KEAGMEVELF |
| [Clostridium butyricum | 61 EGVEPDPSVE TVMKGAEAMR | shown), 160G, 189V, | 61 EGVEPDPSVE TVMKGAEAMR |
| 60E.3]. | KFQPDWIVAM GGGSPIDAAK AMWIFYEYPD | 189C (alternative; not | KFQPDWIVAM GGGSPIDAAK AMWIFYEYPD |
| ACCESSION ENZ30152 | FTFEQAVVPF | shown), 194F, 200G | FTFEQAVVPF |
| VERSION ENZ30152.1 | 121 GLPELRQKAK FVAIPSTSGT ATEVTAFSVI | (alternative; not shown), | 121 GLPELRHKAK FVDIPSTSGT ATEVTAFSVI |
| GI:480687150 | TNYTEKIKYP LADFNITPDI AIVDPELAQT | 200T, 264Q, 264V | TNYTEKIKYG LADFNITPDI AIVDPELAQT |
| | 181 MPAKLVAHTG MDALTHAIEA YTASLRSNFS | (alternative; not shown), | 181 MPAKLVAHVG MDAFTHAIET YTASLRSNFS |
| | DPLAIKAIEM VNENLVKSFE GDKTARNLMH | 264L (alternative; not | DPLAIKAIEM VHENLVKSFE GDKTARNLMH |
| | 241 EAQCLAGMAF SNALLGIVHS | shown), 264S | 241 EAQCLAGMAF SNALLGIVHS |
| | MAHKVGAVFH IPHGCANAIF LPYVIKYNRK | (alternative; not shown), | MAHQVGPVFH IPHGCATAIF LPYVIKYNRK |
| | VCEDRYADIA | 267P, 277T, 360S, 206R, | VCEDRYADIA |
| | 301 RALKLEGNTD AELTDSLIKL INEFNDDLSI | 222H, 127H, 133D, 334L | 301 RALKLEGNTD AELTDSLIKL INEFNDDLSI |
| | PHSMKEYGVE EADFKSNVKF IAHNAILDAC | | PHSLKEYGVE EADFKSNVKF IAHNAILDAS |
| | 361 TGSSPREIDE ETMEKLFECT YYGTDVNF (SEQ | | 361 TGSSPREIDE ETMEKLFECT YYGTDVNF |
| | ID NO: 217) | | (SEQ ID NO: 229) |
| iron-containing alcohol | 1 MARFTLPRDL YHGEGALEVL KTLKGKKAFV | 34H, 35A, | 1 MARFTLPRDL YHGEGALEVL KTLKGKKAFV |
| dehydrogenase | VVGGGSMKRF GFLQKVENYL KEAGMEVELF | 35K (alternative; not | VVGHASMKRF GFLQKVENYL KEAGMEVELF |
| [Clostridium | 61 EGVEPDPSVE TVMKGAEAMR NFEPDWIVSI | shown), 160G, 189V, | 61 EGVEPDPSVE TVMKGAEAMR NFEPDWIVSI |
| saccharoperbutylacetonicum | GGGSPIDAAK AMWIFYEYPD FTFEQAVVPF | 189C (alternative; not | GGGSPIDAAK AMWIFYEYPD FTFEQAVVPF |
| N1-4(HMT)] | 121 GLPELRQKAK FVAIPSTSGT ATEVTAFSVI | shown), 194F, 200G | 121 GLPELRHKAK FVDIPSTSGT ATEVTAFSVI |
| NCBI Reference Sequence: | TNYTERIKYP LADFNITPDI AIIDPELAQT | (alternative; not shown), | TNYTERIKYG LADFNITPDI AIIDPELAQT |
| YP_007455800.1 | 181 MPKTLTAHTG MDALTHAIEA YTASLRSNFS | 200T, 264Q, 264V | 181 MPKTLTAHVG MDAFTHAIET YTASLRSNFS |
| | DPLAIKAIEM VNENLFKSYE GDKEARNLMH | (alternative; not shown), | DPLAIKAIEM VHENLFKSYE GDKEARNLMH |
| | 241 EAQCLAGMAF SNALLGIVHS | 264L (alternative; not | 241 EAQCLAGMAF SNALLGIVHS |
| | MAHKVGAVFH IPHGCANAIF LPYVIQYNRT | shown), 264S | MAHQVGPVFH IPHGCATAIF LPYVIQYNRT |
| | KCEERYADIA | (alternative; not shown), | KCEERYADIA |
| | 301 RALKLEGNND AELTDALIAR INKLNEKLSI | 267P, 277T, 360S, 206R, | 301 RALKLEGNND AELTDALIAR INKLNEKLSI |
| | PSSMKEYGVT EEDFKANLSF IAHNAVLDAC | 222H, 127H, 133D, 334L | PSSLKEYGVT EEDFKANLSF IAHNAVLDAS |
| | 361 TGSNPREIDD ATMEKLFECT FYGTKVEL (SEQ | | 361 TGSNPREIDD ATMEKLFECT FYGTKVEL |
| | ID NO: 218) | | (SEQ ID NO: 230) |
| iron-containing alcohol | 1 MARFTLPRDI YHGEGALEAL KTLKGKKAFL | 34H, 35A, 35K | 1 MARFTLPRDI YHGEGALEAL KTLKGKKAFL |
| dehydrogenase | VVGGGSMKRF GFLKQVEDYL KEAGMEVELF | (Alternative; not shown), | VVGHASMKRF GFLKQVEDYL KEAGMEVELF |
| [Clostridium beijerinckii | 61 EGVEPDPSVE TVMKGAEAMR | 160G, 189V, 189C | 61 EGVEPDPSVE TVMKGAEAMR |
| NCIMB 8052] | NFEPDWIVAM GGGSPIDAAK AMWIFYEYPD | (Alternative; not shown), | NFEPDWIVAM GGGSPIDAAK AMWIFYEYPD |
| NCBI Reference Sequence: | FTFEQAVVPF | 194F, 200G (Alternative; | FTFEQAVVPF |
| YP_001309304.1 | 121 GLPDLRQKAK FVAIPSTSGT ATEVTAFSVI | not shown), 200T, 264Q, | 121 GLPDLRHKAK FVDIPSTSGT ATEVTAFSVI |
| | TNYSEKIKYP LADFNITPDI AIVDPALAQT | 264V (Alternative; not | TNYSEKIKYG LADFNITPDI AIVDPALAQT |
| | 181 MPKTLTAHTG MDALTHAIEA YTASLQSNFS | shown), 264L | 181 MPKTLTAHVG MDAFTHAIET YTASLRSNFS |
| | DPLAIKAVEM VQENLIKSFE GDKEARNLMH | (Alternative; not shown), | DPLAIKAVEM VHENLIKSFE GDKEARNLMH |
| | 241 EAQCLAGMAF SNALLGIVHS | 264S (Alternative; not | 241 EAQCLAGMAF SNALLGIVHS |
| | MAHKVGAVFH IPHGCANAIF LPYVIEYNRT | shown), 267P, 277T, | MAHQVGPVFH IPHGCATAIF LPYVIEYNRT |
| | KCENRYGDIA | 360S, 206R, 222H, 127H, | KCENRYGDIA |
| | 301 RALKLKGNND AELTDSLIEL INGLNDKLEI | 133D, 334L | 301 RALKLKGNND AELTDSLIEL INGLNDKLEI |
| | PHSMKEYGVT EEDFKANLSF IAHNAVLDAC | | PHSLKEYGVT EEDFKANLSF IAHNAVLDAS |
| | 361 TGSNPREIDD ATMEKLFECT YYGTKVNL | | 361 TGSNPREIDD ATMEKLFECT YYGTKVNL |
| | (SEQ ID NO: 219) | | (SEQ ID NO: 231) |
| hypothetical protein | 1 MSRFTLPRDI YYGANALEAL KTLKGKKAVM | 34H, 35A, 35K | 1 MSRFTLPRDI YYGANALEAL KTLKGKKAVM |
| BACCAP_02245 | VLGGGSMKRF GFVDKAIGYL KEAGIETKLF | (alternative; not shown), | VLGHASMKRF GFVDKAIGYL KEAGIETKLF |
| [Bacteroides capillosus | 61 ENVEPDPSVE TVMKGAAMMR EFEPDWIISM | 160G, 189V, T189C | 61 ENVEPDPSVE TVMKGAAMMR |
| ATCC 29799] | GGGSPIDAAK AMWAFYEYPE TTFEDLITPF | (alternative; not shown), | EFEPDWIISM GGGSPIDAAK AMWAFYEYPE |
| Sequence ID: | 121 NFPELRQKAK FCAIPSTSGT ATEVTAFSVI | 194F, 200G (alternative; | TTFEDLITPF |
| ref\|ZP_02036635.1\| | TDYAKGIKYP LADFNITPDV AIVDPALAET | not shown), 200T, 264Q, | 121 NFPELRHKAK FCDIPSTSGT ATEVTAFSVI |
| | 181 MPPHLVAYTG MDALTHAIEA YVSTLHTVFT | 264V (alternative; not | TDYAKGIKYG LADFNITPDV AIVDPALAET |
| | DPLAIEAIHI VEKDLIKSFK GDMTCREEMH | shown), 264L | 181 MPPHLVAYVG MDAFTHAIET YVSTLRTVFT |
| | 241 YGQCLAGMAF SNALLGIVHS MAHKTGAAFS | (alternative; not shown), | DPLAIEAIHI VHKDLIKSFK GDMTCREEMH |
| | TGHITHGLAN AMYLPYVIAY NSKADGVAER | 264S (alternative; not | 241 YGQCLAGMAF SNALLGIVHS |
| | 301 YADIARTMGV VGDTTAELVE GLRAKIRSMN | shown), 267P, 280T, | MAHQTGPAFS TGHITHGLAT AMYLPYVIAY |
| | DAMGIPNTLK DFGIIEDEFK EKLEAIATNA | 360S, 206R, 222H, 127H, | NSKADGVAER |
| | 361 VGDACTGSNP RPIDPATMAK LFTCIYYGTE | 133D, 339L | 301 YADIARTMGV VGDTTAELVE GLRAKIRSMN |
| | VDF (SEQ ID NO: 220) | | DAMGIPNTLK DFGIIEDEFK EKLEAIATNA |
| | | | 361 VGDASTGSNP RPIDPATMAK LFTCIYYGTE |
| | | | VDF (SEQ ID NO: 232) |
| NADPH-dependent | 1 MGGIISMARF TLPRDIYHGK DSLEVLKSLE | 40H, 41A, 41K | 1 MGGIISMARF TLPRDIYHGK DSLEVLKSLE |
| butanol dehydrogenase | GKKAFIVIGG GSMKRFGFLD KVLSYLKEAN | (alternative; not shown), | GKKAFIVIGH |
| [Clostridium perfringens E | 61 METKVFEGVE PDPSVETVMK GAKEMEEFNP | 166G, 195V, 195C | ASMKRFGFLD KVLSYLKEAN |
| str. JGS1987] | DWIVSIGGGS PIDAAKAMWI FYEYPDFTFE | (alternative; not shown), | 61 METKVFEGVE PDPSVETVMK |
| Sequence ID: | 121 KAIVPFGLPK LRRKAKFVAI PSTSGTATEV | 200F, 206G (alternative; | GAKEMEEFNP DWIVSIGGGS PIDAAKAMWI |
| ref\|ZP_02631505.2\| | TAFSVITDYK AKIKYPLADF EITPDIAIVD | not shown), 206T, 270Q, | FYEYPDFTFE |
| Length: 394 | 181 PSLAETMPEK LVAHTGMDAL THAIEAYTAS | 270V (alternative; not | 121 KAIVPFGLPK LRHKAKFVDI PSTSGTATEV |
| | LRSNFTDPLA LKAIEMVNMH LVNSFKGDME | shown), 270L | TAFSVITDYK AKIKYGLADF EITPDIAIVD |
| | 241 ARGEMHEAQC LAGMAFSNAL | (alternative; not shown), | 181 PSLAETMPEK LVAHVGMDAF THAIETYTAS |
| | LGIVHSMAHK VGAVFHIPHG CANAIFLPYV | 270S (alternative; not | LRSNFTDPLA LKAIEMVHMH LVNSFKGDME |
| | IKYNRKACED | shown), 273P, 283T, | 241 ARGEMHEAQC LAGMAFSNAL |
| | 301 RYAQIARHIG LKGESERELT DALIDLINKF | 366S, 212R, 228H, 133H, | LGIVHSMAHQ VGPVFHIPHG CATAIFLPYV |
| | NKELNIPSSM KEYGIDENEF KTNLKFIAHN | 139D, 340L | IKYNRKACED |
| | 361 AVLDPCTGSN PREIDDETME KLYTCAYYGS | | 301 RYAQIARHIG LKGESERELT DALIDLINKF |
| | DVDF (SEQ ID NO: 221) | | NKELNIPSSM KEYGIDENEF KTNLKFIAHN |
| | | | 361 AVLDPSTGSN PREIDDETME KLYTCAYYGS |
| | | | DVDF (SEQ ID NO: 233) |

Exemplary ADH variants include, but are not limited to single substitutions, or a combination of one or more of the substitutions, at 34H, 35A, 35K, 161G, 190V, 190C, 190H, 195F, 201G, 201T, 201L, 201F, 265Q, 265V, 265L, 265S, 268P, 278T, 361S, 207R, 223H, 128H, 134D, 335L for representative sequence(s) shown in Table 56; 34H, 35A, 35K, 160G, 189V, 189C, 189A, 194F, A200G, 200T, 200S, 264Q, 264V, 264L, 264S, 267P, 282T, 377S, 206R, 222H, 127H, 133D, 334L for representative sequence(s) shown in Table 56; 34H, 35A, 35K, 160G, 189V, 189C, 194F, 200G, 200T, 264Q, 264V, 264L, 264S, 264A, 267P, 280T, 363S, 206R, 222H, 127H, 133D, 337L for representative sequence(s) shown in Table 56; 34H, 35A, 35K, 160G, 189V, 189C, 194F, 200G, 200T, 264Q, 264V, 264L, 264S, 267P, 280T, 360S, 206R, 222H, 127H, 133D, 334L for representative sequence(s) shown in Table 56; 34H, 35A, 35K, 160G, 189V, 189C, 194F, 200G, 200T, 264Q, 264V, 264L, 264S, 267P, 277T, 360S, 206R, 222H, 127H, 133D, 334L for representative sequence(s) shown in Table 56; 34H, 35A, 35K, 160G, 189V, 189C, 194F, 200G, 200T, 264Q, 264V, 264L, 264S, 267P, 277T, 360S, 206R, 222H, 127H, 133D, 334L for representative sequence(s) shown in Table 56; 34H, 35A, 35K, 160G, 189V, 189C, 194F, 200G, 200T, 264Q, 264V, 264L, 264S, 267P, 277T, 360S, 206R, 222H, 127H, 133D 334L for representative sequence(s) shown in Table 56; 34H, 35A, 35K, 160G, 189V, 189C, 194F, 200G, 200T, 264Q, 264V, 264L, 264S, 267P, 277T, 360S, 206R, 222H, 127H, 133D, 334L for representative sequence(s) shown in Table 56; 34H, 35A, 35K, 160G, 189V, 189C, 194F, 200G, 200T, 264Q, 264V, 264L, 264S, 267P, 277T, 360S, 206R, Q222H, Q127H, A133D, M334L for representative sequence(s) shown in Table 56; 34H, 35A, 35K, 160G, 189V, T189C, 194F, 200G, 200T, 264Q, 264V, 264L, 264S, 267P, 280T, 360S, 206R, 222H, 127H, 133D, 339L for representative sequence(s) shown in Table 56; 40H, 41A, 41K, 166G, 195V, 195C, 200F, 206G, 206T, 270Q, 270V, 270L, 270S, 273P, 283T, 366S, 212R, 228H, 133H, 139D, 340L for representative sequence(s) shown in Table 56. Representative combinations include combinations of the single substitutions exemplified in Table 56 as in the corresponding respective sequences, including but not limited to: 34H, 35A; 34H, 160G; 35A, 160G; 35K, 160G; 133D, 206R, 334L; 267P, 206R, 222H, 127H, 133D; Q222H, A133D, M334L; Q222H, Q127H, A133D, M334L; 200T, 267P; 160G, 189V, 200G, 267P; 160G, 189V, 200T, 267P; 160G, 189C, 200G, 267P; 160G, 189C, 200T, 267P; 160G, 200T; 160G, 267P; 360S, 206R, 222H, 127H; 160G, 189V, 200T, 267P, 360S; 160G, 189C, 200T, 267P, 360S; 160G, 200G, 267P, 277T, 360S, 206R, 222H, 127H, 133D, 334L; 160G, 200T, 267P, 277T, 360S, 206R, 222H, 127H, 133D, 334L; 160G, 200G, 267P, 277T, 360S; 160G, 200T, 267P, 277T, 360S; 160G, 200G, 267P; 160G, 200T, 267P; 360S, 222H, 133D; 360S, 127H, 133D; 127H, 133D; 127H, 133D, 200T; 133D, 200T; 200T, 267P; 194F, 200G, 264Q, 264V; 206R, 222H; 34H, 222H; 35A, 160G, 200T, 267P; 160G, 200T, 267P; 160G, 200G, 267P, 277T, 360S; 160G, 200G, 267P, C360S; 160G, 200G; 160G, 200T; 160G, 267P; 200G, 267P; 200G, 267P, 277T, 360S, 206R, 222H, 127H, 133D, 334L; 200T, 267P, 277T, 360S, 206R, 222H, 127H, 133D, 334L; 200G, 267P, 360S, 206R, 222H, 127H, 133D, 334L; 200T, 267P, 360S, 206R, 222H, 127H, 133D, 334L; 127H, 200T; 200G, 267P, 206R, 222H, 127H, 133D, 334L; 200T, 267, 206R, 222, 127H, 133D, 334L; 200G, 267P, 222H, 127H, 133D, 334L; 200T, 267P, 222H, 127H, 133D, 334L; 200G, 267P, 127H, 133D, 334L; 200T, 267P, 127H, 133D, 334L; 200G, 267P, 334L; 200T, 267P, 334L; 200G, 334L; 200T, 334L; 267P, 334L; 200T, 267P, 334L; 200G, 133D; 200T, 133D; 34H, 35A, 160G, 194F; 34H, 35K, 160G, 194F; 34H, 160G, 189V, 194F; 34H, 160G, 189C, 194F; 34H, 189V, 194F, 200G, 334L; 34H, 189V, 194F, 200T, 334L; 34H, 189C, 194F, 200G, 334L; 34H, 189C, 194F, 200T, 334L; 34H, 194F, 267P, 360S, 334L; 34H, 194F, 267P, 360S; 34H, 264Q, 277T, 360S; 34H, 264V/, 277T, 360S; 34H, 264L, 277T, 360S; 34H, 264S, 277T, 360S; 194F, 200T, 267P, 222H; 34H, 35A, 160G, 189C, 194F, 200T, 264Q, 267P, 277T, 360S; 34H, 35A, 160G, 189V, 194F, 200T, 264V, 267P, 277T, 360S; 34H, 35K, 160G, 189V, 194F, 200T, 264L, 267P, 277T, 360S; G34H, 35K, 160G, 189V, 194F, 200T, 264S, 267P, 277T, 360S; 34H, 35K, 160G, 189V, 194F, 200T, 264Q, 267P, 277T; 34H, 35K, 160G, 189V, 194F, 200T, 264Q, 267P; 35K, 160G, 189V, 194F, 200T, 267P; 160G, 189C, 194F, 200T, 267P; 160G, 189V, 200T, 267P; 34H, G35K, 160G, 189V, 200T, 264Q, 267P, 277T, 360S, 206R 160G, 189C, 194F, 200T, 264Q, 267P, 360S, 334L; 194F, 264Q; 194F, 264V; 194F, 264L; 194F, 264S; 200G, 360S; 200T, 360S; 200G, 206R; 200T, 206R; 200T, 222H; 200G, 127H, 133D, 334L; 200T, 127H, 133D, 334L; 277T, 360S, 206R, 222H, 127H, 133D, 334L; 360S, 206R, 222H, 127H, 133D, 334L; 206R, 222H, 127H, 133D, 334L; 222H, 127H, 133D, 334L; 127H, 133D, 334L; 133D, 334L; 160G, 133D, 334L; 200G, 277T, 360S, 206R, 222H, 127H, 133D; 200T, 277T, 360S, 206R, 222H, 127H, 133D; 200G, 277T, 360S, 206R, 222H, 127H; 200T, 277T, 360S, 206R, 222H, 127H; 200G, 277T, 360S, 206R, 222H; 200T, 277T, 360S, 206R, 222H; 200G, 277T, 360S, 206R 200T, 277T, 360S, 206R; 200G, 277T, 360S; 200T, 277T, 360S; 194F, 360S, 334L; 194F, 360S, 206R, 222H, 127H, 133D, 334L; 194F, 200T, 264Q, 264V; 166G, 195C, 200F; 160G, 200T, 267P; 34H, 35A, 223H, 128H; 267P, 360S, 206R 277T, 360S, 206R 34H, 35A, 200G; 40H, 195V, 206T, 270V, and the like. It is readily apparent that these an other combinations can be readily made, used and screened using methods well known in the art and as disclosed herein. Based on methods well known in the art for aligning homologous sequences, as described herein, the corresponding positions for the indicated variant amino acid positions of Table 56 can be readily determined for a homologous ADH sequence.

Variants of ALD were generated using site-directed mutagenesis and/or saturation mutagenesis and/or error-prone PCR and/or directed evolution. Variants were screened for production of products and/or byproducts such as BDO or ethanol in high-throughput assays or in in vitro assays as well. Additional variants were generated by random combinations of mutations. At least 179 variants were identified having activity for producing BDO and 4HB, and the variants exhibited production of at least about the same or higher concentrations of 4HB than BDO. At least the same or increased activities were observed in both in vitro and in vivo assays for numerous variants. Certain variants exhibited activity of about 2-fold or greater than the wild type parental enzyme, for example, about 2.5-fold, about 3-fold, about 3.5-fold, about 5-fold, and about 6-fold greater than wild type. Variants with combinations of two or more variant amino acid positions exhibited activities greater than wild type. Generating enzyme variants by combining active variant amino acid positions resulted in enzyme variants with improved properties.

Exemplary screening methods for ALD activity include, for example, the above in vivo methods for measuring BDO and/or ethanol production as well as a 4-hydroxybutyrate (4HB) consumption assay or an in vivo method for measuring BDO, 4-hydroxybutyrate (4HB) and/or gamma-butyrolactone (GBL) production.

The 4-hydroxybutyrate (4HB) consumption assay was performed by 300 μl of M9-MOPS-antibiotic medium containing IPTG (0.2 mM for induction of protein expression) and 4-hydroxybutyrate (50 mM)) in a 96-well plate were aseptically inoculated with 6 μl glycerol stock of the clone harboring the plasmid with the aldehyde dehydrogenase gene. A sealing mat was placed on top and the whole plate wrapped in a gas-tight pouch to create a micro-aerobic environment. Cells were then grown under shaking at 30° C. for 20 hours. After measuring the OD of the grown culture, cells were removed by centrifugation and the supernatant used for quantification of the remaining 4HB. To quantify 4HB, an enzymatic reaction was applied in which 4HB is oxidized with NAD+ to succinate semialdehyde using a succinic semialdehyde dehydrogenase. The concentration of remaining 4HB is determined by extrapolation to a standard curve with defined 4HB concentrations. Aldehyde dehydrogenase (Ald) variants were also evaluated for 4-hydroxybutyryl-CoA reductase activity by an enzyme-coupled assay, where the first enzyme is carboxylic acid transferase 2 (Cat2) and the second enzyme is the test Ald variant. Lysates were typically prepared by chemical lysis containing lysozyme, nuclease, 10 mM DTT, and with or without 1 mM coenzyme A. The standard screening assay solution contained 0.1 M Tris-HCl, pH 6.5, 20 mM 4-hydroxybutyric acid, 2 mM acetyl-CoA, 0.25 mM NADH, and 0.25 mM NADPH. Alternatively, the screening assay was conducted without 4HB. The reaction was initiated by the addition of an enzyme lysate that contained both Cat2 and the Ald variant. Initial rate was determined by measuring the linear decrease in cofactor fluorescence (excitation wavelength=360 nm; emission wavelength=465 nm) using a microtiter plate reader.

The in vivo assay for measuring production of BDO, 4-hydroxybutyrate (4HB) and/or gamma-butyrolactone (GBL) was performed by aseptically inoculating a 96 deep well plate with 1 ml of LB+required antibiotic(s) with the colonies of interest. Plates were covered with a gas permeable adhesive seal and incubated overnight (ON) with shaking in a humidified incubator at 37° C. The next day, 100 μl of the ON culture was transferred into a new 96 well plate, containing 1 ml of M9-MOPS-antibiotic medium per well. After a 5-6 hour incubation (as described above), the plate was centrifuged at 4000 rpm for 5 minutes and the supernatant removed. 1.2 ml fresh M9-MOPS-antibiotic was added to each well, and incubated ON as described above. After the incubation, an aliquot of each culture was used to measure OD at 600 nm. The 96 well plate was centrifuged and the supernatants vacuum-filtered with a 96 well plate AcroPrep filter to remove remaining cells. The filtrate was collected and analyzed by LCMS or HPLC.

Table 57 provides exemplary ALD sequences based on homology. One skilled in the art will readily understand that such sequences can be analyzed with routine and well known methods for aligning sequences (for example BLAST, blast.ncbi.nlm.nih.gov; Altschul et al., “J. Mol. Biol. 215:403-410 (1990)). Such alignments can provide information on conserved residues that can be utilized to identify a consensus sequence for preserving enzyme activity as well as positions for generating further enzyme variants.

TABLE 57
Exemplary ALD sequences.

| | |
|---|---|
| NAD-dependent aldehyde dehydrogenase [Clostridium saccharoperbutylacetonicum N1-4(HMT)] >gb\|AGF59413.1\| NAD- | YP_007458667.1 |
| dependent aldehyde dehydrogenase [Clostridium saccharoperbutylacetonicum N1-4(HMT)] | GI: 451822466 |
| | (SEQ ID NO: 439) |
| butyraldehyde dehydrogenase [Clostridium saccharoperbutylacetonicum N1-4(HMT)] | AAP42563.1 |
| | GI: 31075383 |
| | (SEQ ID NO: 440) |
| aldehyde dehydrogenase [Clostridium beijerinckii NCIMB 8052] >gb\|AAQ12068.1\| coenzyme A acylating aldehyde | YP_001310903.1 |
| dehydrogenase [Clostridium beijerinckii NCIMB 8052] >gb\|AAQ12072.1\| coenzyme A acylating aldehyde dehydrogenase | GI: 150018649 |
| [Clostridium beijerinckii] >gb\|AAT48939.1\| aldehyde dehydrogenase [Clostridium beijerinckii] >gb\|AAT66436.1\| coenzyme | (SEQ ID NO: 441) |
| acylating aldehyde dehydrogenase [Clostridium beijerinckii] >gb\|ABR35947.1\| aldehyde dehydrogenase [Clostridium beijerinckii | |
| NCIMB 8052] | |
| coenzyme A acylating aldehyde dehydrogenase [Clostridium beijerinckii] | AAD31841.1 |
| | GI: 4884855 |
| | (SEQ ID NO: 442) |
| Acetaldehyde dehydrogenase (acetylating) [Clostridium sp. DL-VIII] >gb\|EHJ00721.1\| Acetaldehyde dehydrogenase | ZP_09206127.1 |
| (acetylating) [Clostridium sp. DL-VIII] | GI: 359413662 |
| | (SEQ ID NO: 443) |
| coenzyme A acylating aldehyde dehydrogenase [Clostridium saccharobutylicum] | CAQ57983.1 |
| | GI: 189310620 |
| | (SEQ ID NO: 444) |
| ethanolamine utilization protein EutE [Clostridium botulinum B str. Eklund 17B] >gb\|ACD24339.1\| ethanolamine utilization | YP_001886323.1 |
| protein EutE [Clostridium botulinum B str. Eklund 17B] | GI: 187934965 |
| | (SEQ ID NO: 445) |
| Aldehyde Dehydrogenase [Caldalkalibacillus thermarum TA2.A1] >gb\|EGL82399.1\| Aldehyde Dehydrogenase | ZP_08533507.1 |
| [Caldalkalibacillus thermarum TA2.A1] | GI: 335040377 |
| | (SEQ ID NO: 446) |
| Aldehyde Dehydrogenase [Pelosinus fermentans DSM 17108] >ref\|ZP_15517111.1\| Aldehyde Dehydrogenase [Pelosinus | ZP_10327808.1 |
| fermentans B4] >ref\|ZP_15521980.1\| Aldehyde Dehydrogenase [Pelosinus fermentans B3] >ref\|ZP_15526533.1\| Aldehyde | GI: 392962372 |
| Dehydrogenase [Pelosinus fermentans A12] >ref\|ZP_15534416.1\| Aldehyde Dehydrogenase [Pelosinus fermentans | (SEQ ID NO: 447) |
| A11] >gb\|EIW18982.1\| Aldehyde Dehydrogenase [Pelosinus fermentans B4] >gb\|EIW21808.1\| Aldehyde Dehydrogenase | |
| [Pelosinus fermentans A11] >gb\|EIW29163.1\| Aldehyde Dehydrogenase [Pelosinus fermentans DSM 17108] >gb\|EIW35484.1\| | |
| Aldehyde Dehydrogenase [Pelosinus fermentans B3] >gb\|EIW36902.1\| Aldehyde Dehydrogenase [Pelosinus fermentans A12] | |
| NAD-dependent aldehyde dehydrogenase [Thermoanaerobacterium thermosaccharolyticum M0795] >gb\|AGB19701.1\| NAD- | YP_007299398.1 |
| dependent aldehyde dehydrogenase [Thermoanaerobacterium thermosaccharolyticum M0795] | GI: 433655690 |
| | (SEQ ID NO: 448) |
| Aldehyde Dehydrogenase [Pelosinus fermentans JBW45] >gb\|EIW48189.1\| Aldehyde Dehydrogenase [Pelosinus fermentans | ZP_15537951.1 |
| JBW45] | GI: 421076976 |
| | (SEQ ID NO: 449) |
| aldehyde dehydrogenase family protein [Desulfosporosinus sp. OT] >gb\|EGW35902.1\| aldehyde dehydrogenase family protein | ZP_08814704.1 |
| [Desulfosporosinus sp. OT] | GI: 345862484 |
| | (SEQ ID NO: 450) |
| hypothetical protein CLOSTMETH_00016 [Clostridium methylpentosum DSM 5476] >gb\|EEG32278.1\| hypothetical protein | ZP_03705305.1 |
| CLOSTMETH_00016 [Clostridium methylpentosum DSM 5476] | GI: 225016072 |
| | (SEQ ID NO: 451) |
| aldehyde dehydrogenase [Thermoanaerobacterium saccharolyticum JW/SL-YS485] >gb\|AFK85255.1\| Aldehyde Dehydrogenase | YP_006390854.1 |
| [Thermoanaerobacterium saccharolyticum JW/SL-YS485] | GI: 390933349 |
| | (SEQ ID NO: 452) |
| acetaldehyde dehydrogenase [Thermoanaerobacterium xylanolyticum LX-11] >gb\|AEF18105.1\| Acetaldehyde dehydrogenase | YP_004471777.1 |
| (acetylating) [Thermoanaerobacterium xylanolyticum LX-11] | GI: 333897903 |
| | (SEQ ID NO: 453) |
| aldehyde dehydrogenase EutE [Acetonema longum DSM 6540] >gb\|EGO64744.1\| aldehyde dehydrogenase EutE [Acetonema | ZP_08623980.1 |
| longum DSM 6540] | GI: 338811775 |
| | (SEQ ID NO: 454) |
| ethanolamine utilization protein eutE [Geobacillus thermoglucosidans TNO-09.020] >gb\|EID44455.1\| ethanolamine utilization | ZP_17694107.1 |
| protein eutE [Geobacillus thermoglucosidans TNO-09.020] | GI: 423719925 |
| | (SEQ ID NO: 455) |
| aldehyde dehydrogenase [Geobacillus sp. Y4.1MC1] >gb\|ADP74637.1\| Aldehyde Dehydrogenase [Geobacillus sp. Y4.1MC1] | YP_003989248.1 |
| | GI: 312110932 |
| | (SEQ ID NO: 456) |
| aldehyde dehydrogenase [Geobacillus thermoglucosidans C56-YS93] >>gb\|AEH47899.1\| Aldehyde dehydrogenase | YP_004587980.1 |
| (acetylating) [Geobacillus thermoglucosidasius C56-YS93] | GI: 336235364 |
| | (SEQ ID NO: 457) |
| aldehyde dehydrogenase EutE [Bacillus azotoformans LMG 9581] >gb\|EKN64472.1\| aldehyde dehydrogenase EutE [Bacillus | ZP_11313951.1 |
| azotoformans LMG 9581] | GI: 410460269 |
| | (SEQ ID NO: 458) |
| putative aldehyde dehydrogenase, ethanolamine utilization protein [Clostridium] sticklandii] >emb\|CBH20800.1\| putative | YP_003935705.1 |
| aldehyde dehydrogenase, ethanolamine utilization protein [[Clostridium] sticklandii] | GI: 310657984 |
| | (SEQ ID NO: 459) |
| Aldehyde Dehydrogenase [Thermincola potens JR] >gb\|ADG81503.1\| Aldehyde Dehydrogenase [Thermincola potens JR] | YP_003639404.1 |
| | GI: 296132157 |
| | (SEQ ID NO: 460) |
| CoA-dependent propionaldehyde dehydrogenase [Clostridium sp. D5] >gb\|EGB92558.1\| CoA-dependent propionaldehyde | ZP_08130302.1 |
| dehydrogenase [Clostridium sp. D5] | GI: 325263568 |
| | (SEQ ID NO: 461) |
| acetaldehyde dehydrogenase (acetylating) [Fusobacterium sp. 3_1_33] >gb\|EEW94895.1\| acetaldehyde dehydrogenase | ZP_05815063.1 |
| (acetylating) [Fusobacterium sp. 3_1_33] | GI: 260494934 |
| | (SEQ ID NO: 462) |
| ethanolamine utilization protein eutE [Fusobacterium sp. 7_1] >gb\|EEO43449.1\| ethanolamine utilization protein eutE | ZP_04573939.1 |
| [Fusobacterium sp. 7_1] | GI: 237743458 |
| | (SEQ ID NO: 463) |
| NAD-dependent aldehyde dehydrogenases [Ruminococcus sp. SR1/5] >emb\|CBL20089.1\| NAD-dependent aldehyde | YP_007783752.1 |
| dehydrogenases [Ruminococcus sp. SR1/5] | GI: 479153977 |
| | (SEQ ID NO: 464) |
| hypothetical protein HMPREF9942_01197 [Fusobacterium nucleatum subsp. animalis F0419] >gb\|EHO78009.1\| hypothetical | ZP_17125059.1 |
| protein HMPREF9942_01197 [Fusobacterium nucleatum subsp. animalis F0419] | GI: 423137416 |
| | (SEQ ID NO: 465) |
| possible aldehyde dehydrogenase [Fusobacterium nucleatum subsp. polymorphum ATCC 10953] >gb\|EDK87521.1\| possible | ZP_04969437.1 |
| aldehyde dehydrogenase [Fusobacterium nucleatum subsp. polymorphum ATCC 10953] | GI: 254302079 |
| | (SEQ ID NO: 466) |
| ethanolamine utilization protein eutE [Fusobacterium sp. D11] >gb\|EFD80567.1\| ethanolamine utilization protein eutE | ZP_06524378.1 |
| [Fusobacterium sp. D11] | GI: 289765000 |
| | (SEQ ID NO: 467) |
| aldehyde dehydrogenase EutE [Fusobacterium nucleatum ChDC F128] >gb\|EJU08233.1\| aldehyde dehydrogenase EutE | ZP_15972610.1 |
| [Fusobacterium nucleatum ChDC F128] | GI: 421526001 |
| | (SEQ ID NO: 468) |
| CoA-dependent propionaldehyde dehydrogenase [Fusobacterium nucleatum subsp. polymorphum F0401] >gb\|EHG19190.1 | ZP_16419680.1 |
| CoA-dependent propionaldehyde dehydrogenase [Fusobacterium nucleatum subsp. polymorphum F0401] | GI: 422338720 |
| | (SEQ ID NO: 469) |
| CoA-dependent propionaldehyde dehydrogenase [Fusobacterium sp. 11_3_2] >gb\|EGN65750.1\| CoA-dependent | ZP_08600044.1 |
| propionaldehyde dehydrogenase [Fusobacterium sp. 11_3_2] | GI: 336419790 |
| | (SEQ ID NO: 470) |
| hypothetical protein CLOSTASPAR_02210 [Clostridium asparagiforme DSM 15981] >gb\|EEG55710.1\| hypothetical protein | ZP_03758198.1 |
| CLOSTASPAR_02210 [Clostridium asparagiforme DSM 15981] | GI: 225388474 |
| | (SEQ ID NO: 471) |
| aldehyde dehydrogenase [Clostridium phytofermentans ISDg] >gb\|ABX41556.1\| Aldehyde Dehydrogenase_[Clostridium | YP_001558295.1 |
| phytofermentans ISDg] | GI: 160879327 |
| | (SEQ ID NO: 472) |
| CoA-dependent propionaldehyde dehydrogenase [Fusobacterium sp. 1_1_41FAA] >gb\|EFG28139.1\| CoA-dependent | ZP_06748808.1 |
| propionaldehyde dehydrogenase [Fusobacterium sp. 1_1_41FAA] | GI: 294783484 |
| | (SEQ ID NO: 473) |
| hypothetical protein HMPREF0991_01940 [Lachnospiraceae bacterium 2_1_58FAA] >gb\|EGN47419.1\| hypothetical protein | ZP_08612821.1 |
| HMPREF0991_01940 [Lachnospiraceae bacterium 2_1_58FAA] | GI: 336432991 |
| | (SEQ ID NO: 474) |
| hypothetical protein RUMGNA_01022 [Ruminococcus gnavus ATCC 29149] >gb\|EDN78612.1\| aldehyde dehydrogenase | ZP_02040258.1 |
| (NAD) family protein [Ruminococcus gnavus ATCC 29149] | GI: 154503198 |
| | (SEQ ID NO: 475) |
| NAD-dependent aldehyde dehydrogenases [Ruminococcus obeum A2-162] >emb\|CBL23217.1\| NAD-dependent aldehyde | YP_007805199.1 |
| dehydrogenases [Ruminococcus obeum A2-162] | GI: 479177598 |
| | (SEQ ID NO: 476) |
| aldehyde dehydrogenase [Clostridium saccharolyticum WM1] >gb\|ADL04402.1\| Aldehyde Dehydrogenase [Clostridium | YP_003822025.1 |
| saccharolyticum WM1] | GI: 302386203 |
| | (SEQ ID NO: 477) |
| aldehyde dehydrogenase family protein [Flavonifractor plautii ATCC 29863] >gb\|EHM40040.1\| aldehyde dehydrogenase family | ZP_09385796.1 |
| protein [Flavonifractor plautii ATCC 29863] | GI: 365844997 |
| | (SEQ ID NO: 478) |
| hypothetical protein RUMOBE_00094 [Ruminococcus obeum ATCC 29174] >gb\|EDM88971.1\| aldehyde dehydrogenase | ZP_01962381.1 |
| (NAD) family protein [Ruminococcus obeum ATCC 29174] | GI: 153809713 |
| | (SEQ ID NO: 479) |
| Aldehyde Dehydrogenase [Clostridium carboxidivorans P7] >ref\|ZP_06856832.1\| aldehyde dehydrogenase (NAD) family protein | ZP_05391061.1 |
| [Clostridium carboxidivorans P7] >gb\|EET88516.1\| Aldehyde Dehydrogenase [Clostridium carboxidivorans | GI: 255524100 |
| P7] >gb\|EFG86154.1\| aldehyde dehydrogenase (NAD) family protein [Clostridium carboxidivorans P7] >gb\|ADO12117.1\| CoA- | (SEQ ID NO: 480) |
| acylating aldehyde dehydrogenase [Clostridium carboxidivorans P7] | |
| hypothetical protein FUAG_00592 [Fusobacterium ulcerans ATCC 49185] >gb\|EFS25077.1\| hypothetical protein FUAG_00592 | ZP_10974295.1 |
| [Fusobacterium ulcerans ATCC 49185] | GI: 404368948 |
| | (SEQ ID NO: 481) |
| hypothetical protein HMPREF0402_00608 [Fusobacterium sp. 12_1B] >gb\|EHO83590.1\| hypothetical protein | ZP_09586735.1 |
| HMPREF0402_00608 [Fusobacterium sp. 12_1B] | GI: 373496187 |
| | (SEQ ID NO: 482) |
| aldehyde dehydrogenase [Roseburia inulinivorans DSM 16841] | ABC25528.1 |
| | GI: 83596371 |
| | (SEQ ID NO: 483) |
| Aldehyde Dehydrogenase [Clostridium carboxidivorans P7] >ref\|ZP_06855343.1\| aldehyde dehydrogenase (NAD) family protein | ZP_05393779.1 |
| [Clostridium carboxidivorans P7] >gb\|EET85788.1\| Aldehyde Dehydrogenase [Clostridium carboxidivorans | GI: 255526882 |
| P7] >gb\|EFG87815.1\| aldehyde dehydrogenase (NAD) family protein [Clostridium carboxidivorans P7] | (SEQ ID NO: 484) |
| NAD-dependent aldehyde dehydrogenases [Clostridium cf. saccharolyticum K10] >emb\|CBK77787.1\| NAD-dependent | YP_007849785.1 |
| aldehyde dehydrogenases [Clostridium cf. saccharolyticum K10] | GI: 479338567 |
| | (SEQ ID NO: 485) |
| ethanolamine utilization protein eutE [Fusobacterium varium ATCC 27725] >gb\|EES62817.1\| ethanolamine utilization protein | ZP_08693593.1 |
| eutE [Fusobacterium varium ATCC 27725] | GI: 340756989 |
| | (SEQ ID NO: 486) |
| aldehyde dehydrogenase family protein [Clostridium celatum DSM 1785] >gb\|EKY29259.1\| aldehyde dehydrogenase family | ZP_19296595.1 |
| protein [Clostridium celatum DSM 1785] | GI: 429764274 |
| | (SEQ ID NO: 487) |
| propionaldehyde dehydrogenase [Clostridium sp. ASF502] | EMZ20682.1 |
| | GI: 476613570 |
| | (SEQ ID NO: 488) |
| hypothetical protein HMPREF0988_02063 [Lachnospiraceae bacterium 1_4_56FAA] >gb\|EGN36620.1\| hypothetical protein | ZP_08616478.1 |
| HMPREF0988_02063 [Lachnospiraceae bacterium 1_4_56FAA] | GI: 336436768 |
| | (SEQ ID NO: 489) |
| hypothetical protein HMPREF0994_03038 [Lachnospiraceae bacterium 3_1_57FAA_CT1] >gb\|EGN40215.1\| hypothetical | ZP_08607032.1 |
| protein HMPREF0994_03038 [Lachnospiraceae bacterium 3_1_57FAA_CT1] | GI: 336427027 |
| | (SEQ ID NO: 490) |
| aldehyde dehydrogenase [Ruminococcus sp. 5_1_39B_FAA] >gb\|EES77009.1\| aldehyde dehydrogenase [Ruminococcus sp. | ZP_04856816.1 |
| 5_1_39BFAA] | GI: 253579547 |
| | (SEQ ID NO: 491) |
| CoA-dependent proprionaldehyde dehydrogenase PduP [Acetobacterium woodii DSM 1030] >gb\|AFA49334.1\| CoA-dependent | YP_005270223.1 |
| proprionaldehyde dehydrogenase PduP [Acetobacterium woodii DSM 1030] | GI: 379012411 |
| | (SEQ ID NO: 492) |
| ethanolamine utilization protein EutE [Clostridium botulinum El str. ‘BoNT E Beluga’] >gb\|EES50221.1\| ethanolamine utilization | ZP_04822936.1 |
| protein EutE [Clostridium botulinum El str. ‘BoNT E Beluga’] | GI: 251780016 |
| | (SEQ ID NO: 493) |
| ethanolamine utilization protein EutE [Clostridium botulinum B str. Eklund 17B] >gb\|ACD22415.1\| ethanolamine utilization | YP_001885942.1 |
| protein EutE [Clostridium botulinum B str. Eklund 17B] | GI: 187933041 |
| | (SEQ ID NO: 494) |
| ethanolamine utilization protein EutE [Clostridium botulinum E3 str. Alaska E43] >gb\|ACD53952.1\| ethanolamine utilization | YP_001921227.1 |
| protein EutE [Clostridium botulinum E3 str. Alaska E43] | GI: 188590535 |
| | (SEQ ID NO: 495) |
| propionaldehyde dehydrogenase [Eubacterium plexicaudatum ASF492] | EMZ27833.1 |
| | GI: 476621007 |
| | (SEQ ID NO: 496) |
| Aldehyde Dehydrogenase [Thermosediminibacter oceani DSM 16646] >gb\|ADL07333.1\| Aldehyde Dehydrogenase | YP_003824956.1 |
| [Thermosediminibacter oceani DSM 16646] | GI: 302389135 |
| | (SEQ ID NO: 497) |
| hypothetical protein HMPREF1090_01637 [Clostridium clostridioforme 90A8] | ENZ17687.1 |
| | GI: 480674262 |
| | (SEQ ID NO: 498) |
| hypothetical protein HMPREF9467_03550 [Clostridium clostridioforme 2_1_49FAA] >gb\|EHG29726.1\| hypothetical protein | ZP_09116578.1 |
| HMPREF9467_03550 [Clostridium clostridioforme 2_1 49FAA] | GI: 357055510 |
| | (SEQ ID NO: 499) |
| Aldehyde Dehydrogenase [Ilyobacter polytropus DSM 2926] >gb\|ADO84118.1\| Aldehyde Dehydrogenase [Ilyobacter | YP_003968466.1 |
| polytropus DSM 2926] | GI: 310780134 |
| | (SEQ ID NO: 500) |
| hypothetical protein GCWU000342_00651 [Shuttleworthia satelles DSM 14600] >gb\|EEP29295.1\| hypothetical protein | ZP_04454656.1 |
| GCWU000342_00651 [Shuttleworthia satelles DSM 14600] | GI: 229828587 |
| | (SEQ ID NO: 501) |
| aldehyde dehydrogenase [Clostridium beijerinckii NCIMB 8052] >gb\|ABR36155.1\| aldehyde dehydrogenase [Clostridium | YP_001311111.1 |
| beijerinckii NCIMB 8052] | GI: 150018857 |
| | (SEQ ID NO: 502) |
| propionaldehyde dehydrogenase [Clostridium clostridioforme CM201] >gb\|ENZ04399.1\| propionaldehyde dehydrogenase | ENY83847.1 |
| [Clostridium clostridioforme 90B1] >gb\|ENZ17257.1\| propionaldehyde dehydrogenase [Clostridium clostridioforme | GI: 480639338 |
| 90A8] >gb\|ENZ22132.1\| propionaldehyde dehydrogenase [Clostridium clostridioforme 90A3] >gb\|ENZ29200.1\| propionaldehyde | (SEQ ID NO: 503) |
| dehydrogenase [Clostridium clostridioforme 90A1] >gb\|ENZ64224.1\| propionaldehyde dehydrogenase [Clostridium | |
| clostridioforme 90A4] >gb\|ENZ70105.1\| propionaldehyde dehydrogenase [Clostridium clostridioforme 90A6] | |
| aldehyde dehydrogenase (NAD) domain protein [Clostridium sp. MSTE9] >gb\|EJF40077.1\| aldehyde dehydrogenase (NAD) | ZP_14663848.1 |
| domain protein [Clostridium sp. MSTE9] | GI: 420157008 |
| | (SEQ ID NO: 504) |
| hypothetical protein CLOBOL_07248 [Clostridium bolteae ATCC BAA-613] >gb\|EDP12494.1\| hypothetical protein | ZP_02089671.1 |
| CLOBOL_07248 [Clostridium bolteae ATCC BAA-613] | GI: 160942363 |
| | (SEQ ID NO: 505) |
| propionaldehyde dehydrogenase [Clostridium bolteae 90B8] >gb\|ENZ57487.1\| propionaldehyde dehydrogenase [Clostridium | ENZ31577.1 |
| bolteae 90A5] >gb\|ENZ67775.1\| propionaldehyde dehydrogenase [Clostridium bolteae 90B7] | GI: 480688660 |
| | (SEQ ID NO: 506) |
| hypothetical protein EUBHAL_00514 [Eubacterium hallii DSM 3353] >gb\|EEG37590.1\| aldehyde dehydrogenase (NAD) family | ZP_03715465.1 |
| protein [Eubacterium hallii DSM 3353] | GI: 225026273 |
| | (SEQ ID NO: 507) |
| CoA-acylating propionaldehyde dehydrogenase [Halanaerobium saccharolyticum subsp. saccharolyticum DSM 6643] | ZP_23773859.1 |
| >emb\|CCU77919.1\| CoA-acylating propionaldehyde dehydrogenase [Halanaerobium saccharolyticum subsp. saccharolyticum | GI: 470960332 |
| DSM 66431 | (SEQ ID NO: 508) |
| hypothetical protein [Eubacterium limosum KIST612] >gb\|ADO39014.1\| hypothetical protein ELI_4072 [Eubacterium limosum | YP_003961977.1 |
| KIST612] | GI: 310829620 |
| | (SEQ ID NO: 509) |
| aldehyde dehydrogenase [Thermoanaerobacter sp. X514] >ref\|ZP_07131928.1\| Aldehyde Dehydrogenase [Thermoanaerobacter | YP_001663556.1 |
| sp. X561] >ref\|YP_003903905.1\| aldehyde dehydrogenase [Thermoanaerobacter sp. X513] >ref\|ZP_08212082.1\| Aldehyde | GI: 167040571 |
| Dehydrogenase [Thermoanaerobacter ethanolicus JW 200] >gb\|ABY93220.1\| aldehyde dehydrogenase [Thermoanaerobacter sp. | (SEQ ID NO: 510) |
| X514] >gb\|EFK84693.1\| Aldehyde Dehydrogenase [Thermoanaerobacter sp. X561] >gb\|ADN54614.1\| Aldehyde | |
| Dehydrogenase [Thermoanaerobacter sp. X513] >gb\|EGD51928.1\| Aldehyde Dehydrogenase [Thermoanaerobacter ethanolicus | |
| JW 200] | |
| aldehyde dehydrogenase [Rhodospirillum rubrum ATCC 11170] >ref\|YP_006047210.1\| aldehyde dehydrogenase EutE | YP_426002.1 |
| [Rhodospirillum rubrum F11] >gb\|ABC21715.1\| Aldehyde dehydrogenase [Rhodospirillum rubrum ATCC | GI: 83592250 |
| 11170] >gb\|AEO47413.1\| aldehyde dehydrogenase EutE [Rhodospirillum rubrum F11] | (SEQ ID NO: 511) |
| CoA-dependent propionaldehyde dehydrogenase [Eubacterium yurii subsp. margaretiae ATCC 43715] >gb\|EFM39950.1\| CoA- | ZP_07453625.1 |
| dependent propionaldehyde dehydrogenase [Eubacterium yurii subsp. margaretiae ATCC 43715] | GI: 306819974 |
| | (SEQ ID NO: 512) |
| aldehyde dehydrogenase (NAD) domain protein [Eubacterium sp. AS15] >gb\|EJP26117.1\| aldehyde dehydrogenase (NAD) | ZP_10828060.1 |
| domain protein [Eubacterium sp. AS15] | GI: 402309064 |
| | (SEQ ID NO: 513) |
| aldehyde dehydrogenase EutE [Vibrio sp. EJY3] >gb\|AEX22176.1\| aldehyde dehydrogenase EutE [Vibrio sp. EJY3] | YP_005023154.1 |
| | GI: 375265711 |
| | (SEQ ID NO: 514) |
| hypothetical protein HMPREF9629_00032 [Eubacteriaceae bacterium ACC19a] >gb\|EHL16790.1\| hypothetical protein | ZP_09320518.1 |
| HMPREF9629_00032 [Eubacteriaceae bacterium ACC19a] | GI: 363893420 |
| | (SEQ ID NO: 515) |
| aldehyde-alcohol dehydrogenase domain protein [Propionibacterium propionicum F0230a] >gb\|AFN47240.1\| aldehyde-alcohol | YP_006513121.1 |
| dehydrogenase domain protein [Propionibacterium propionicum F0230a] | GI: 397671586 |
| | (SEQ ID NO: 516) |
| hypothetical protein HMPREF9628_01348 [Eubacteriaceae bacterium CM5] >gb\|EHL19659.1\| hypothetical protein | ZP_09316712.1 |
| HMPREF9628_01348 [Eubacteriaceae bacterium CM5] | GI: 363889349 |
| | (SEQ ID NO: 517) |
| aldehyde dehydrogenase (NAD) family protein [Eubacteriaceae bacterium OBRC8] >gb\|EJU23517.1\| aldehyde dehydrogenase | ZP_10886417.1 |
| (NAD) family protein [Eubacteriaceae bacterium OBRC8] | GI: 402837902 |
| | (SEQ ID NO: 518) |

Table 58 provides exemplary ALD enzyme variants. Table 58 shows exemplary enzyme variant positions with the corresponding amino acid for a particular position that were identified as providing a desirable activity, including ALD activity, BDO production, and the like. It is understood that the individual ALD variants such as those described in Table 58 can be used alone, or can be combined with any other variant amino acid position, including 2, 3, 4, 5, 6, 7, 8, 9, 10, greater than 10 and up to all variant amino acid positions as disclosed herein, to generate additional variants having desirable activities.

TABLE 58
Exemplary ALD enzyme variants.

| Wild-Type | | | Exemplary Enzyme Sequence |
|---|---|---|---|
| Designation | Wild-Type Sequence | Corresponding Positions and Amino Acid Changes | Indicating Corresponding Positions |
| aldehyde | 1 MNKDTLIPTT KDLKVKTNGE | D12L, L15P, D30N, F34K, N42D, I44L, A47T, A47V | 1 MNKDTLIPTT KLLKVKTNGE |
| dehydrogenase | NINLKNYKDN SSCFGVFENV | (ALTERNATIVE; NOT SHOWN), D12L, L15P, D30N, | NINLKNYKDN SSCKGVFENV |
| [Clostridium | ENAISSAVHA QKILSLHYTK | F34K, N42D, I44L, A47T, A47V (ALTERNATIVE; NOT | EDALSSTVHA RKILSGACGK |
| beijerinckii] | 61 EQREKIITEI RKAALQNKEV | SHOWN), Q51R, Q51K (ALTERNATIVE; NOT | 61 EQREKIFSEI AKAALQNKEV |
| ACCESSION: | LATMILEETH MGRYEDKILK | SHOWN), L56G, H57A, Y58C, Y58L (ALTERNATIVE; | LATMILEETH RGRYEDKILK |
| AAT48939 | HELVAKYTPG TEDLTTTAWS | NOT SHOWN), Y58H (ALTERNATIVE; NOT SHOWN), | HELRATVTPG TENLTTTAWS |
| VERSION: | 121 GDNGLTVVEM SPYGVIGAIT | Y58L (ALTERNATIVE; NOT SHOWN), T59G, I66F, | 121 GDNGLTINAM WAMGPAGAST |
| AAT48939.1 | PSTNPTETVI CNSIGMIAAG | I67S, I67A (ALTERNATIVE; NOT SHOWN), R71A, | PLTNPTETVI CNSIGCIVGG |
| GI:49036681 | NAVVFNGHPC AKKCVAFAVE | M91R, V104R, K106T, Y107V, D113N, V127I, | NAVAFNPHPC AGKCVAYGVE |
| | 181 MINKAIISCG GPENLVTTIK | V128N, E129A, E129G (ALTERNATIVE; NOT | 181 MINMAIKGCG GPLNLVTTIM |
| | NPTMESLDAI IKHPSIKLLC | SHOWN), E129A (ALTERNATIVE; NOT SHOWN), | NPTLESLAAI CKHPMIQLLC |
| | GTGGPGMVKT LLNSGKKAIG | E129Y (ALTERNATIVE; NOT SHOWN), E129I | GTGGPRLVAV ILQGGKKAIG |
| | 241 AGAGNPPVIV DDTADIEKAG | (ALTERNATIVE; NOT SHOWN), S131W, P132A, | 241 SSRGNPPTIV DSFADIERTS |
| | RSIIEGCSFD NNLPCIAEKE | Y133M, Y133V (ALTERNATIVE; NOT SHOWN), | RSIIISCSLD NNIPCIAETS LFVPENVLKD |
| | VFVFENVADD LISNMLKNNA | Y133M (ALTERNATIVE; NOT SHOWN), V135P, | VISVFLKLNA |
| | 301 VIINEDQVSK LIDLVLQKNN | I36A, I139S, I139T (ALTERNATIVE; NOT SHOWN), | 301 VIINEDQVSL LIDLVLAKNN |
| | ETQEYFINKK WVGKDAKLFL | S142L, M156C, A158V, A159G, V164A, G167P, | ETQEYFKNKK WVAKDAKLFL |
| | DEIDVESPSN VKCIICEVNA | K172G, F177Y, A178G, K184M, I187K, S188G, | DEIDVESPWN VKNIACEVNE |
| | 361 NHPFVMTELM MPILPIVRVK | E193L, K200M, M204L, M204R,(ALTERNATIVE; | 361 NHPFVMTELL MPILPIVRVK |
| | DIDEAIKYAK IAEQNRKHSA | NOT SHOWN), D208A, D208L (ALTERNATIVE; NOT | DIYEAIKYAK IAEQPHKFSA |
| | YIYSKNIDNL NRFEREIDTT | SHOWN), I211C, S215M, S215A (ALTERNATIVE; | MIYSKHIDNL NRFEREIDTT |
| | 421 IFVKNAKSFA GVGYEAEGFT | NOT SHOWN), K217Q, G226R, M227L, M227I | 421 IFVKNAKSFA GVGYEAEGFT |
| | TFTIAGSTGE GITSARNFTR | (ALTERNATIVE; NOT SHOWN), K229A, T230V, | TFTIAGSTGE GITSARNFTR QRRCVLAG |
| | QRRCVLAG (SEQ ID NO: 234) | T230R (ALTERNATIVE; NOT SHOWN), T230Q | (SEQ ID NO: 241) |
| | | (ALTERNATIVE; NOT SHOWN), T230V | |
| | | (ALTERNATIVE; NOT SHOWN), L231I, N233Q, | |
| | | N233V (ALTERNATIVE; NOT SHOWN), S234G, | |
| | | S234G (ALTERNATIVE; NOT SHOWN), A241S, | |
| | | G242S, G242D (ALTERNATIVE; NOT SHOWN), | |
| | | A243R, A243N (ALTERNATIVE; NOT SHOWN), | |
| | | A243E (ALTERNATIVE; NOT SHOWN), V248T, | |
| | | D252S, T253F, T253L (ALTERNATIVE; NOT | |
| | | SHOWN) T253V (ALTERNATIVE; NOT SHOWN) | |
| | | T253A (ALTERNATIVE; NOT SHOWN) K258R, | |
| | | A259T, G260S, G260T (ALTERNATIVE; NOT | |
| | | SHOWN), G260C (ALTERNATIVE; NOT SHOWN), | |
| | | G260V (ALTERNATIVE; NOT SHOWN), E265I, E265C | |
| | | (ALTERNATIVE; NOT SHOWN), E265W | |
| | | (ALTERNATIVE; NOT SHOWN), E265V | |
| | | (ALTERNATIVE; NOT SHOWN), G266S, F269L, L273I, | |
| | | L273T (ALTERNATIVE; NOT SHOWN), L273S | |
| | | (ALTERNATIVE; NOT SHOWN), L273M | |
| | | (ALTERNATIVE; NOT SHOWN), L273N | |
| | | (ALTERNATIVE; NOT SHOWN), K279T, E280S, | |
| | | V281L, F284P, A288L, D289K, L291V, N294V, | |
| | | N294D (ALTERNATIVE; NOT SHOWN), M295F, | |
| | | N298L, K310L, K310P (ALTERNATIVE; NOT | |
| | | SHOWN), Q317A, Q317T (ALTERNATIVE; NOT | |
| | | SHOWN), I327K, G333A, S349W, C353N, 1355A, | |
| | | A360E, M370L, M370H (ALTERNATIVE; NOT | |
| | | SHOWN), D383Y, N395P, R396H R396K | |
| | | (ALTERNATIVE; NOT SHOWN), R396W | |
| | | (ALTERNATIVE; NOT SHOWN), R396V | |
| | | (ALTERNATIVE; NOT SHOWN), H398F, | |
| | | Y401M, N406H | |
| ethanolamine | 1 MERNLSVLSQ TNDLKITKRT | D13L, I16P, L32K, N40D, I42L, A45T, A45V | 1 MERNLSVLSQ TNLKPTKRT |
| utilization | EGDKSNNKES YLGVFKKVEN | (alternative; not shown), Q49R, Q49K (alternative; | EGDKSNNKES YKGVFKKVED |
| protein EutE | AITKAIYAQK KLSLYYTKED | not shown), L54G, Y55A, Y56C, Y56L (alternative; | ALTKTIYARK KLSGACGKED |
| [Clostridium | 61 RERIIKSIRK ATLENKEILA | not shown), Y56H (alternative; not shown), Y56L | 61 RERFSKSIAK ATLENKEILA |
| botulinum B | KMIVDETHMG RYEDKILKHE | (alternative; not shown), T57G, I64F, I65S, I65A | KMIVDETHRG RYEDKILKHE |
| str. Eklund | LVAKYTPGTE DLITTAWSGD | (alternative; not shown), R69A, M89R, V102R, | LRATVTPGTE NLITTAWSGD |
| 17B] | 121 QGLTLVEMSP YGVIGAITPS | K104T, Y105V, D111N, V125I, V126N, E127A, | 121 QGLTINAMWA MGPAGASTPL |
| Sequence ID: | TNPTETVICN SIGMIAAGDS | E127G (alternative; not shown), E127Y | TNPTETVICN SIGCIVGGDS |
| ref\| | VVFNGHPGAK KCVAFAVDMI | (alternative; not shown), E127I (alternative; not | VAFNPHPGAG KCVAYGVDMI |
| YP_001886323.1\| | 181 NKAVIREGGP ENLVTTVENP | shown), S129W, P130A, Y131M, Y131V | 181 NMAVKSEGGP LNLVTTVMNP |
| Length: 467 | TMESLNVIMK HPYIKLLCGT | (alternative; not shown), Y131M (alternative; not | TLESLAVICK HPMIQLLCGT GGPRLIAVIL |
| | GGPGLIKTLL NSGKKAIGAG | shown), V133P, I134A, I137S, I137T (alternative; | QGKKAIGSS |
| | 241 AGNPPVIVDD SADIDKAAKN | not shown), S140L, M154C, A156V, A157G, V162A, | 241 RGNPPTIVDS FADIDRTSKN |
| | IIEGCSFDNN LPCIAEKEVF | G165P, K170G, F175Y, A176G, K182M, 1185K, | IIISCSLDNN IPCIAETSLF VPENVLNKVI |
| | VFENVANDLI QNMIKNNAVL | S186G, E191L, K198M, M202L, M202R (alternative; | QVFIKLNAVL |
| | 301 INENQVSKLL DLVLLERKDE | not shown), N206A, N206L (alternative; not | 301 INENQVSLLL DLVLLARKDE |
| | TLEYAINKKW VGKDAKLFLD | shown), M209C, Y213M, Y213A (alternative; not | TLEYAKNKKW VAKDAKLFLD |
| | KIGIKASDNV RCIICEVDAN | shown), K215Q, G224R, L225I (alternative; not | KIGIKASWNV RNIACEVDEN |
| | 361 HPFVMTELMM PILPIVRVKD | shown), K227A, T228V, T228R (alternative; not | 361 HPFVMTELLM PILPIVRVKD |
| | VDEAIECAKT AEQRKRHSAY | shown), T228Q (alternative; not shown), T228V | VYEAIECAKT AEQPKRFSAM |
| | MYSKNIDNLN RFEKEIDTTI | (alternative; not shown), L229I, N231Q, N231V | MYSKHIDNLN RFEKEIDTTI |
| | 421 FVKNAKSFAG VGFGAEGFTT | (alternative; not shown), S232G, A239S, G240S, | 421 FVKNAKSFAG VGFGAEGFTT |
| | FTIAGPTGEG ITSARNFTRQ RRCVLAG | G240D (alternative; not shown), A241R, A241N | FTIAGPTGEG ITSARNFTRQ RRCVLAG |
| | (SEQ ID NO: 235) | (alternative; not shown), A241E (alternative; not | (SEQ ID NO: 242) |
| | | shown), V246T, D250S, T251F, T251L (alternative; | |
| | | not shown), T251V (alternative; not shown), | |
| | | T251A (alternative; not shown), K256R, A257T, | |
| | | G258S, G258T (alternative; not shown), G260C | |
| | | (alternative; not shown), G260V (alternative; not | |
| | | shown), E263I, E263C (alternative; not shown), | |
| | | E263W (alternative; not shown), E263V | |
| | | (alternative; not shown), G264S, F267L, L271I, | |
| | | L271T (alternative; not shown), L271S (alternative; | |
| | | not shown), L271M (alternative; not shown), | |
| | | L271N (alternative; not shown), K277T, E278S, | |
| | | V279L, F282P, A286L, D287K, L289V, N292V, | |
| | | N292D (alternative; not shown), M293F, N296L, | |
| | | K308L, K308P (alternative; not shown), E316A, | |
| | | E316T (alternative; not shown), I326K, G332A, | |
| | | D348W, C352N, I354A, A359E, M369L, M369H | |
| | | (alternative; not shown), D382Y, R394P, K395H, | |
| | | K395K (alternative; not shown), K395W | |
| | | (alternative; not shown), K395V (alternative; not | |
| | | shown), H397F, Y400M, N405H | |
| aldehyde | 1 MNKDTLIPTT KDLKVKTNGE | D12L, L15P, D30N, F34K, N42D, I44L, A47T, A47V | 1 MNKDTLIPTT KLLKVKTNGE |
| dehydrogenase | NINLKNYKDN SSCFGVFENV | (ALTERNATIVE; NOT SHOWN), D12L, L15P, D30N, | NINLKNYKDN SSCKGVFENV |
| [Clostridium | ENAISSAVHA QKILSLHYTK | F34K, N42D, I44L, A47T, A47V (ALTERNATIVE; NOT | EDALSSTVHA RKILSGACGK |
| beijerinckii] | 61 EQREKIITEI RKAALQNKEV | SHOWN), Q51R, Q51K (ALTERNATIVE; NOT | 61 EQREKIFSEI AKAALQNKEV |
| ACCESSION: | LATMILEETH MGRYEDKILK | SHOWN), L56G, H57A, Y58C, Y58L (ALTERNATIVE; | LATMILEETH RGRYEDKILK |
| AAT48939 | HELVAKYTPG TEDLTTTAWS | NOT SHOWN), Y58H (ALTERNATIVE; NOT SHOWN), | HELRATVTPG TENLTTTAWS |
| VERSION: | 121 GDNGLTVVEM SPYGVIGAIT | Y58L (ALTERNATIVE; NOT SHOWN), T59G, I66F, | 121 GDNGLTINAM WAMGPAGAST |
| AAT48939.1 | PSTNPTETVI CNSIGMIAAG | I67S, I67A (ALTERNATIVE; NOT SHOWN), R71A, | PLTNPTETVI CNSIGCIVGG |
| GI:49036681 | NAVVFNGHPC AKKCVAFAVE | M91R, V104R, K106T, Y107V, D113N, V127I, | NAVAFNPHPC AGKCVAYGVE |
| | 181 MINKAIISCG GPENLVTTIK | V128N, E129A, E129G (ALTERNATIVE; NOT | 181 MINMAIKGCG GPLNLVTTIM |
| | NPTMESLDAI IKHPSIKLLC | SHOWN), E129A (ALTERNATIVE; NOT SHOWN), | NPTLESLAAI CKHPMIQLLC |
| | GTGGPGMVKT LLNSGKKAIG | E129Y (ALTERNATIVE; NOT SHOWN), E129I | GTGGPRLVAV ILQGGKKAIG |
| | 241 AGAGNPPVIV DDTADIEKAG | (ALTERNATIVE; NOT SHOWN), S131W, P132A, | 241 SSRGNPPTIV DSFADIERTS |
| | RSIIEGCSFD NNLPCIAEKE | Y133M, Y133V (ALTERNATIVE; NOT SHOWN), | RSIIISCSLD NNIPCIAETS LFVPENVLKD |
| | VFVFENVADD LISNMLKNNA | Y133M (ALTERNATIVE; NOT SHOWN), V135P, | VISVFLKLNA |
| | 301 VIINEDQVSK LIDLVLQKNN | I136A, I139S, I139T (ALTERNATIVE; NOT SHOWN), | 301 VIINEDQVSL LIDLVLAKNN |
| | ETQEYFINKK WVGKDAKLFL | S142L, M156C, A158V, A159G, V164A, G167P, | ETQEYFKNKK WVAKDAKLFL |
| | DEIDVESPSN VKCIICEVNA | K172G, F177Y, A178G, K184M, I187K, S188G, | DEIDVESPWN VKNIACEVNE |
| | 361 NHPFVMTELM MPILPIVRVK | E193L, K200M, M204L, M204R (ALTERNATIVE; | 361 NHPFVMTELL MPILPIVRVK |
| | DIDEAIKYAK IAEQNRKHSA | NOT SHOWN), D208A, D208L (ALTERNATIVE; NOT | DIYEAIKYAK IAEQPHKFSA |
| | YIYSKNIDNL NRFEREIDTT | SHOWN), I211C, S215M, S215A (ALTERNATIVE; | MIYSKHIDNL NRFEREIDTT |
| | 421 IFVKNAKSFA GVGYEAEGFT | NOT SHOWN), K217Q, G226R, M227L, M227I | 421 IFVKNAKSFA GVGYEAEGFT |
| | TFTIAGSTGE GITSARNFTR | (ALTERNATIVE; NOT SHOWN), K229A, T230V, | TFTIAGSTGE GITSARNFTR QRRCVLAG |
| | QRRCVLAG (SEQ ID NO: 236) | T230R (ALTERNATIVE; NOT SHOWN), T230Q | (SEQ ID NO: 243) |
| | | (ALTERNATIVE; NOT SHOWN), T230V | |
| | | (ALTERNATIVE; NOT SHOWN), L231I, N233Q, | |
| | | N233V (ALTERNATIVE; NOT SHOWN), S234G, | |
| | | S234G (ALTERNATIVE; NOT SHOWN), A241S, | |
| | | G242S, G242D (ALTERNATIVE; NOT SHOWN), | |
| | | A243R, A243N (ALTERNATIVE; NOT SHOWN), | |
| | | A243E (ALTERNATIVE; NOT SHOWN), V248T, | |
| | | D252S, T253F, T253L (ALTERNATIVE; NOT | |
| | | SHOWN), T253V (ALTERNATIVE; NOT SHOWN), | |
| | | T253A (ALTERNATIVE; NOT SHOWN), K258R, | |
| | | A259T, G260S, G260T (ALTERNATIVE; NOT | |
| | | SHOWN), G260C (ALTERNATIVE; NOT SHOWN), | |
| | | G260V (ALTERNATIVE; NOT SHOWN), E265I, E265C | |
| | | (ALTERNATIVE; NOT SHOWN), E265W | |
| | | (ALTERNATIVE; NOT SHOWN), E265V | |
| | | (ALTERNATIVE; NOT SHOWN), G266S, F269L, L273I, | |
| | | L273T (ALTERNATIVE; NOT SHOWN), L273S | |
| | | (ALTERNATIVE; NOT SHOWN), L273M | |
| | | (ALTERNATIVE; NOT SHOWN), L273N | |
| | | (ALTERNATIVE; NOT SHOWN), K279T, E280S, | |
| | | V281L, F284P, A288L, D289K, L291V, N294V, | |
| | | N294D (ALTERNATIVE; NOT SHOWN), M295F, | |
| | | N298L, K310L, K310P (ALTERNATIVE; NOT | |
| | | SHOWN), Q317A, Q317T (ALTERNATIVE; NOT | |
| | | SHOWN), I327K, G333A, S349W, C353N, I355A, | |
| | | A360E, M370L, M370H (ALTERNATIVE; NOT | |
| | | SHOWN), D383Y, N395P, R396H, R396K | |
| | | (ALTERNATIVE; NOT SHOWN), R396W | |
| | | (ALTERNATIVE; NOT SHOWN), R396V | |
| | | (ALTERNATIVE; NOT SHOWN), H398F, Y401M, | |
| | | N406H | |
| AD-dependent | 1 MIKDTLVSIT KDLKLKTNVE | D12L, L15P, D30N, F34K, N42D, I44L, A47T, A47V | 1 MIKDTLVSIT KLLKPKTNVE |
| aldehyde | NANLKNYKDD SSCFGVFENV | (alternative; not shown), Q51R, Q51K | NANLKNYKDN SSCKGVFENV |
| dehydrogenase | ENAISNAVHA QKILSLHYTK | (alternative; not shown), L56G, H57A, Y58C, | EDALSNTVHA RKILSGACGK |
| [Clostridium | 61 EQREKIITEI RKAALENKEI | Y58L (alternative; not shown), Y58H (alternative; | 61 EQREKFSTEI AKAALENKEI |
| saccharo- | LATMILEETH MGRYEDKILK | not shown), Y58L (alternative; not shown), | LATMILEETH RGRYEDKILK |
| perbutylacetonicum | HELVAKYTPG TEDLTTTAWS | T59G, I66F, I67S, I67A (alternative; not shown), | HELRATVTPG TENLTTTAWS |
| N1-4(HMT)] | 121 GDNGLTVVEM SPYGVIGAIT | R71A, M91R, V104R, K106T, Y107V, D113N, | 121 GDNGLTINAM WAMGPAGAST |
| Sequence ID: | PSTNPTETVI CNSIGMIAAG | V127I, V128N, E129A, E129G (alternative; not | PLTNPTETVI CNSIGCIVGG |
| ref\| | NTVVFNGHPG AKKCVAFAVE | shown), E129Y (alternative; not shown), E129I | NTVAFNPHPG AGKCVAYGVE |
| YP_007458667.1\| | 181 MINKAIISCG GPENLVTTIK | (alternative; not shown), S131W, P132A, | 181 MINMAIKGCG GPLNLVTTIM |
| Length: 468 | NPTMDSLDAI IKHPSIKLLC | Y133M, Y133V (alternative; not shown), Y133M | NPTLDSLAAI CKHPMIQLLC |
| | GTGGPGMVKT LLNSGKKAIG | (alternative; not shown), V135P, I136A, I139S, | GTGGPRLVAV ILQGGKKAIG |
| | 241 AGAGNPPVIV DDTADIEKAG | I139T (alternative; not shown), S142L, M156C, | 241 SSRGNPPTIV DSFADIERTG |
| | KSIIEGCSFD NNLPCIAEKE | A158V, A159G, V164A, G167P, K172G, F177Y, | KSIIISCSLD NNIPCIAETS LFVPENVLKD |
| | VFVFENVADD LISNMLKNNA | A178G, K184M, I187K, S188G, E193L, K200M, | VISVFLKLNA |
| | 301 VIINEDQVSK LIDLVLQKNN | M204L, M204R (alternative; not shown), | 301 VIINEDQVSL LIDLVLAKNN |
| | ETQEYSINKK WVGKDAKLFL | D208A, D208L (alternative; not shown), I211C, | ETQEYSKNKK WVAKDAKLFL |
| | DEIDVESPSS VKCIICEVSA | S215M, S215A (alternative; not shown), K217Q, | DEIDVESPWS VKCIACEVSE |
| | 361 SHPFVMTELM MPILPIVRVK | G226R, M227L, M227I (alternative; not shown), | 361 SHPFVMTELL MPILPIVRVK |
| | DIDEAIEYAK IAEQNRKHSA | K229A, T230V, T230R (alternative; not shown), | DIYEAIEYAK IAERPHKFSA MIYSKHIDNL |
| | YIYSKNIDNL NRFEREIDTT | T230Q (alternative; not shown), T230V | WRFERGADST |
| | 421 IFVKNAKSFA GVGYEAEGFT | (alternative; not shown), L231I, N233Q, N233V | 421 IFVKNGLSLA GVGYEAPGFT |
| | TFTIAGSTGE GITSARNFTR | (alternative; not shown), S234G, A241S, A241S | SFTIAVPTGE GMTSAANFTR |
| | QRRCVLAG (SEQ ID NO: 237) | (alternative; not shown), G242S, G242D | QRRTVNAN (SEQ ID NO: 244) |
| | | (alternative; not shown), A243R, A243N | |
| | | (alternative; not shown), A243E (alternative; | |
| | | not shown), V248T, D252S, T253F, T253V | |
| | | (alternative; not shown), T253L (alternative; not | |
| | | shown), T253A (alternative; not shown), K258R, | |
| | | A259T, G260S, G260T (alternative; not shown), | |
| | | G260C (alternative; not shown), G260V | |
| | | (alternative; not shown), E265I, E265C | |
| | | (alternative; not shown), E265W (alternative; | |
| | | not shown), E265V (alternative; not shown), | |
| | | G266S, F269L, L273I, L273T, L273S (alternative; | |
| | | not shown), L273M (alternative; not shown), | |
| | | L273T (alternative; not shown), L273N | |
| | | (alternative; not shown), K279T, E280S, V281L, | |
| | | F284P, A288L, D289K, L291V, N294V, N294D | |
| | | (alternative; not shown), M295F, N298L, K310L, | |
| | | K310P (alternative; not shown), Q317A, Q317T | |
| | | (alternative; not shown), I327K, G333A, S349W, | |
| | | C353N, I355A, A360E, M370L, M370H | |
| | | (alternative; not shown), D383Y, N395P, R396H, | |
| | | R396K (alternative; not shown), R396W | |
| | | (alternative; not shown), R396V (alternative; not | |
| | | shown), H398F, Y401M, N406H | |
| Aldehyde | 1 MSIDQALIEK ITLEILTKMQ | E40D, V42L, A45T, A45V (alternative; not shown), | 1 MSIDQALIEK ITLEILTKMQ |
| Dehydrogenase | TGAKAAPAGY GDGIFETVDE | Y49R, Y49K (alternative; not shown), T54G, L55C, | TGAKAAPAGY GDGIFETVDD |
| [Pelosinus | AVAAARKAYQ ELKTLSLEKR | L55H (alternative; not shown), L55L (alternative; | ALAATRKARQ ELKGCGLEKR |
| fermentans | 61 EVLIKAMRDV AYENATILAQ | not shown), S56G, L63F, I64S, I64A (alternative; | 61 EVFSKAMADV AYENATILAQ |
| JBW45] | MAVDESGMGR VSDKIIKNQV | not shown), R68A, M88R, A101R, L103T, K104V, | MAVDESGRGR VSDKIIKNQV |
| Sequence ID: | AALKTPGTED LTTQAWSGDN | D110N, L1241, I125N, E126A, E126G (alternative; | RATVTPGTEN LTTQAWSGDN |
| ref\| | 121 GLTLIEMGPY GVIGAITPTT | not shown), E126Y (alternative; not shown), E126I | 121 GLTINAMWAM GPAGASTPLT |
| ZP_15537951.1\| | NPTETVICNG IGMIAAGNTV | (alternative; not shown), G128W, P129A, Y130M, | NPTETVICNG IGCIVGGNTA |
| Length: 480 | FFSPHPTAKN TSMKIITLLN | Y130V (alternative; not shown), V132P, 1133A, | FFSPHPTAGN TSMYGITLLN |
| | 181 QAIVKAGGPN NLLTSVANPS | I136S, I136T (alternative; not shown), T139L, | 181 MAIKGAGGPL NLLTSVMNPS |
| | IKAANEMMKH PGINMLVATG | M153C, A155V, A156G, V160A, K169G, K174Y, | LKAAAEMCKH PMIQMLVATG |
| | GPGVVKAVLS SGKKAIGAGA | I175G, Q181M, V184K, K185G, N190L, A197M, | GPRLVAVILQ GGKKAIGSSR |
| | 241 GNPPVIVDET ADIEKAARDI | I201L, I201R (alternative; not shown), N205A, | 241 GNPPTIVSEF ADIERTSRDI |
| | VAGCSFDNNL PCIAEKEVIA | N205L (alternative; not shown), M208C, G212M, | VISCSLDNNI PCIAETSLIA PGSILKRVIT |
| | IGSIADRLIT YMQKYGAYLI | G212A (alternative; not shown), N214Q, G223R, | VFQKLGAYLI |
| | 301 SGSNIDRLLN VIMTVQEEKI | V224L, V224I (alternative; not shown), K226A, | 301 SGSNIDRLLN VIMAVQEEKI |
| | AEGCTDKPKR SYGINKDYVG | A227V, A227R (alternative; not shown), A227Q | AEGCTDKPKR SYGKNKDYVA |
| | KDAKYLLSKI GIDVPDSVRV | (alternative; not shown), A227V (alternative; not | KDAKYLLSKI GIDVPDWVRN |
| | 361 VLCETPADHP FVIEELMMPV | shown), V228I, S230Q, S230V (alternative; not | 361 VACETPEDHP FVIEELHMPV |
| | LPVVQVKDID EAIEVAVRVE | shown), S231G, S231G (alternative; not shown), | LPVVQVKYID EAIEVAVRVE |
| | HGNRHTAAMH SKNVDHLTRF | A238S, G239S, G239D (alternative; not shown), | HPHRFTAMMH SKHVDHLTRF |
| | 421 ARAVETTIFV KNAPSYAGIG | A240R, A240N (alternative; not shown), A240E | 421 ARAVETTIFV KNAPSYAGIG |
| | VGGEGFTSFT LAGPTGEGIT | (alternative; not shown), V245T, D248S, T250F, | VGGEGFTSFT LAGPTGEGIT |
| | SPRSFTRQRR CVLVDAFSIV (SEQ ID | T250L (alternative; not shown), T250V | SPRSFTRQRR CVLVDAFSIV (SEQ ID |
| | NO: 238) | (alternative; not shown), T250A (alternative; not | NO: 245) |
| | | shown), K255R, A256T, A257S, A257T (alternative; | |
| | | not shown), A257C (alternative; not shown), | |
| | | A257V (alternative; not shown), A262I, A262C | |
| | | (alternative; not shown), A262W (alternative; not | |
| | | shown), A262V (alternative; not shown), G263S, | |
| | | F266L, L270I, L270T (alternative; not shown), | |
| | | L270S (alternative; not shown), L270M | |
| | | (alternative; not shown), L270N (alternative; not | |
| | | shown), K276T, E277S, V278L, I281P, A285L, | |
| | | D286K, L288V, Y291V, Y291D (alternative; not | |
| | | shown), M292F, Y295L, R308L, R308P (alternative; | |
| | | not shown), T314A, I334K, G340A, S357W, V360N, | |
| | | L362A, A367E, M377L, M377H (alternative; not | |
| | | shown), D388Y, G402P, N403H, N403K | |
| | | (alternative; not shown), N403W (alternative; not | |
| | | shown), N403V (alternative; not shown), H405F, | |
| | | A408M, N413H | |
| coenzyme A | 1 MNKDTLIPTT KDLKLKTNVE | D12L, L15P, F34K, N42D, I44L, A47T, A47V | 1 MNKDTLIPTT KLLKPKTNVE |
| acylating | NINLKNYKDN SSCFGVFENV | (alternative; not shown), Q51R, Q51K (alternative; | NINLKNYKDN SSCKGVFENV |
| aldehyde | ENAINSAVHA QKILSLHYTK | not shown), L56G, H57A, Y58C, Y58L (alternative; | EDALNSTVHA RKILSGACGK |
| dehydrogenase | 61 EQREKIITEI RKAALENKEV | not shown), Y58H (alternative; not shown), Y58L | 61 EQREKFSTEI AKAALENKEV |
| [Clostridium | LATMILEETH MGRYEDKILK | (alternative; not shown), T59G, I66F, I67S, 167A | LATMILEETH RGRYEDKILK |
| beijerinckii] | HELVAKYTPG TEDLTTTAWS | (alternative; not shown), R71A, M91R, V104R, | HELRATVTPG TENLTTTAWS |
| Sequence ID: | 121 GDNGLTVVEM SPYGVIGAIT | K106T, Y107V, D113N, V127I, V128N, E129A, | 121 GDNGLTINAM WAMGPAGAST |
| gb\|AAD31841.1\| | PSTNPTETVI CNSIGMIAAG | E129G (alternative; not shown), E129A | PLTNPTETVI CNSIGCIVGG |
| AF132754_1 | NAVVFNGHPG AKKCVAFAIE | (alternative; not shown), E129Y (alternative; not | NAVAFNPHPG AGKCVAYGIE |
| | 181 MINKAIISCG GPENLVTTIK | shown), E129I (alternative; not shown), S131W, | 181 MINMAIKGCG GPLNLVTTIM |
| | NPTMESLDAI IKHPLIKLLC | P132A, Y133M, Y133V (alternative; not shown), | NPTLESLAAI CKHPMIQLLC |
| | GTGGPGMVKT LLNSGKKAIG | V135P, I136A I139S, I139T (alternative; not | GTGGPRLVAV ILQGGKKAIG |
| | 241 AGAGNPPVIV DDTADIEKAG | shown), S142L, M156C, A158V, A159G, V164A, | 241 SSRGNPPTIV DSFADIERTG |
| | KSIIEGCSFD NNLPCIAEKE | G167P, K172G, F177Y, A178G, K184M, 1187K, | KSIIISCSLD NNIPCIAETS LFVPENVLKD |
| | VFVFENVADD LISNMLKNNA | S188G, E193L, K200M, M204L, M204R (alternative; | VISVFLKLNA |
| | 301 VIINEDQVSK LIDLVLQKNN | not shown), D208A, D208L (alternative; not | 301 VIINEDQVSL LIDLVLAKNN |
| | ETQEYFINKK WVGKDAKLFS | shown), I211C, L215M, S215A (alternative; not | ETQEYFKNKK WVAKDAKLFS |
| | DEIDVESPSN IKCIVCEVNA | shown), K217Q, G226R, M227L, M227I | DEIDVESPWN IKCVACEVNE |
| | 361 NHPFVMTELM MPILPIVRVK | (alternative; not shown), K229A, T230V, T230R | 361 NHPFVMTELL MPILPIVRVK |
| | DIDEAVKYTK IAEQNRKHSA | (alternative; not shown), T230Q (alternative; not | DIYEAVKYTK IAERPHKFSA |
| | YIYSKNIDNL NRFEREIDTT | shown), T230V (alternative; not shown), L231I, | MIYSKHIDNL WRFERGADST |
| | 421 IFVKNAKSFA GVGYEAEGFT | N233Q, N233V (alternative; not shown), S234G, | 421 IFVKNGLSLA GVGYEAPGFT |
| | TFTIAGSTGE GITSARNFTR | A241S, G242S, G242D (alternative; not shown), | SFTIAVPTGE GMTSAANFTR |
| | QRRCVLAG (SEQ ID NO: 239) | A243R, A243N (alternative; not shown), A243E | QRRTVNAN (SEQ ID NO: 246) |
| | | (alternative; not shown), V248T, D252S, T253F, | |
| | | T253L (alternative; not shown), T253V | |
| | | (alternative; not shown), T253L (alternative; not | |
| | | shown), T253A (alternative; not shown), K258R, | |
| | | A259T, G260S, G260T (alternative; not shown), | |
| | | G260C (alternative; not shown), G260V | |
| | | (alternative; not shown), E265I, E265C (alternative; | |
| | | not shown), E265W (alternative; not shown), | |
| | | E265V (alternative; not shown), G266S, F269L, | |
| | | L273I, L273T (alternative; not shown), L273S | |
| | | (alternative; not shown), L273M (alternative; not | |
| | | shown), L273N (alternative; not shown), K279T, | |
| | | E280S, V281L, F284P, A288L, D289K, L291V, | |
| | | N294V, N294D (alternative; not shown), M295F, | |
| | | N298L, K310L, K310P (alternative; not shown), | |
| | | Q317A, Q317T (alternative; not shown), 1327K, | |
| | | G333A, S349W, C353N, V355A, A360E, M370L, | |
| | | M370H (alternative; not shown), D383Y, N395P, | |
| | | R396H, R396K (alternative; not shown), R396W | |
| | | (alternative; not shown), R396V (alternative; not | |
| | | shown), H398F, Y401M, N406H | |
| Aldehyde | 1 MNMTEKDIEK IVQSVLHNVE | I133R, A146R, L148T, K149V, D155N, V170N, | 1 MNMTEKDIEK IVQSVLHNVE |
| Dehydrogenase | SALGKSASAS PSVSAVSVAS | E171A, E171G (alternative; not shown), E171Y | SALGKSASAS PSVSAVSVAS GEGIKPVQFK |
| [Caldalkali- | GEGIKPVQFK QVPVFQQETV | (alternative; not shown), E171I (alternative; not | QVPVFQQETV |
| bacillus | 61 KSPNRNRNLG GAEEKWGVFN | shown), A173W, P174A, Y175M, Y175V | 61 KSPNRNRNLG GAEEKWGVFN |
| thermarum | HMEDAIEASY RAQMEFVKHF | (alternative; not shown), L177P, I178A, V181S, | HMEDAIEASY RAQMEFVKHF |
| TA2.A1] | QLKDREKIIT AIREAVLREK | V181T (alternative; not shown), V184L, M198C, | QLKDREKIIT AIREAVLREK |
| Sequence ID: | 121 EVLARKVYEE TKIGRYEDKV | A200V, A201G, V206A, V209P, K214G, F219Y, | 121 EVLARKVYEE TKRGRYEDKV |
| ref\| | AKHELAALKT PGTEDLKTEA | A220G, K226M, T229K, E230G, H235L, K242M, | AKHELRATVT PGTENLKTEA FSGDNGLTIN |
| ZP_08533507.1\| | FSGDNGLTIV ERAPYGLIGA | L246R, Q250A, Q250L (alternative; not shown), | ARWAMGPAGA |
| Length: 527 | 181 VTPVTNPTET IINNAIGMLA | 1253C, K257M, K257A (alternative; not shown), | 181 STPLTNPTET IINNAIGCLV |
| | AGNAVVFNVH PSSKRSCAYA | K259Q, G268R, L269I, Q271A, T272V, T272R | GGNAVAFNPH PSSGRSCAYG |
| | VQLINKAITE AGGPHHLVTM | (alternative; not shown), T272Q (alternative; not | VQLINMAIKG AGGPLHLVTM |
| | 241 VKEPTLDTLQ TLIDSPKVKL | shown), T272V (alternative; not shown), L273I, | 241 VMEPTRDTLA TLCDSPMVQL |
| | LVGTGGPGLV QTLLKSGKKA | K275Q, K275V (alternative; not shown), S276G, | LVGTGGPRIV AVILQGGKKA |
| | IGAGAGNPPV IVDDTADLEH | A283S, G284S, G284D (alternative; not shown), | IGSSRGNPPT IVDSFADLER |
| | 301 AARSIIEGAA FDNNLLCIAE | A285R, A285N (alternative; not shown), A285E | 301 TSRSIISGAA LDNNILCIAE |
| | KEVFVLESVA DDLIFHMLNH | (alternative; not shown), V290T, D294S, T295F, | TSLFVPESVL KDVIFVFLNL GAYMLGQHEV |
| | GAYMLGQHEV EQVMAFALEE | T295L (alternative; not shown), T295V | ELVMAFALAE |
| | 361 QGNEQNRGCG | (alternative; not shown), T295A (alternative; not | 361 QGNEQNRGCG FNPQRHYQKS |
| | FNPQRHYQVS KDWIGQDARL | shown), H300R, A301T, A302S, A302T (alternative; | KDWIAQDARL FLEHIGVQPP |
| | FLEHIGVQPP TEVKLLICDV | not shown), A302C (alternative; not shown), | WEVKLNICDV EEDHPFVQLE |
| | EFDHPFVQLE | A302V (alternative; not shown), E307I, E307C | 421 QMMPVLPIVR VKTLDEAIEK |
| | 421 QMMPVLPIVR VKTLDEAIEK | (alternative; not shown), E307W (alternative; not | AVMAEHGNRH TAIMHSKNVD |
| | AVMAEHGNRH TAIMHSKNVD | shown), E307V (alternative; not shown), G308S, | HLTKFARAIQ TTLFVKNASS |
| | HLTKFARAIQ TTLFVKNASS | F311L, L315I, L315T (alternative; not shown), | 481 LAGVGYGGEG HTTMTIAGPT |
| | 481 LAGVGYGGEG HTTMTIAGPT | L315S (alternative; not shown), L315M | GEGVTSAKTF TRERRCVLAE GGFRIIG |
| | GEGVTSAKTF TRERRCVLAE GGFRIIG | (alternative; not shown), L315N (alternative; not | (SEQ ID NO: 247) |
| | (SEQ ID NO: 240) | shown), K321T, E322S, V323L, L326P, A330L, | |
| | | D331K, L333V, H336V, H336D (alternative; not | |
| | | shown), M337F, H340L, Q352L, Q352P (alternative; | |
| | | not shown), E359A, E359T (alternative; not | |
| | | shown), V379K, G385A, T401W, L405N, 1407A, | |
| | | F412E | |

Exemplary ALD variants include, but are not limited to, single substitutions, or a combination of one or more of the substitutions, at 12L, 15P, 34K, 42D, 44L, 47T, 51R, 51R, 51K, 56G, 57A, 58C, 58L, 58H, 58L, 59G, 66F, 67S, 67A, 71A, 91R, 104R, 106T, 107V, 127I, 128N, 129A, 129G, 129Y, 129I, 131W, 132A, 133M, 133V, 133M, 135P, 136A, 139S, 139T, 142L, 156C, 158V, 159G, 164A, 167P, 172G, 178G, 184t, 187K, 188G, 200M, 204L, 204R, 208A, 208L, 211C, 215M, 215A, 217Q, 226R, 227L, 227I, 229A, 230V, 230R, 230R, 230Q, 230V, 231I, 233Q, 233V, 234G, 241S, 242S, 242D, 243R, 243N, 243E, 248T, 252S, 253F, 253L, 253V, 253L, 253A, 258R, 259T, 260S, 260T, 260C, 2651, 265C, 265W, 265V, 266S, 269L, 273I, 273T, 273S, 273M, 273T, 273N, 279T, 280S, 281L, 284P, 288L, 289K, 291V, 294V, 295F, 298L, 310L, 310P, 317A, 317T, 327K, 333A, 349W, 360E, 370L, 370H, 395P, 396H, 396K, 396W, 396V, 398F, 401M, 406H, 411W, 416G, 416S, 417A, 419S, 426G, 427L, 427F, 427V, 427L, 429L, 429, 437P, 439H, 441S, 446V, 446I, 447P, 456A, 464T, 464S, 466N, 468N for representative sequence(s) shown in Table 58; 12L, 15P, 34K 42D, 44L, 47T, 47V, 51R, 51R, 51K, 56G, 57A, 58C, 58L, 58H, 59G, 66F, 67S, 67A, 71A, 91R, 104R, 106T, 107V, 113N, 127I, 128N, 129A, 129G, 129Y, 129I, 131W, 132A, 133M, 133V, 133M, 135P, 136A, 139S, 139T, 142L, 156C, 158V, 159G, 164A, 167P, 172G, 177Y, 178G, 184t, 187K, 188G, 193L, 200M, 204L, 204R, 208A, 208L, 211C, 215M, 215A, 217Q, 226R, 227L, 227I, 229A, 230V, 230R, 230R, 230Q, 230V, 231I, 233Q, 233V, 234G, 234G, 241S, 242S, 242D, 243R, 243N, 243E, 248T, 252S, 253F, 253L, 253V, 253A, 258R, 259T, 260S, 260T, 260C, 260V, 2651, 265C, 265W, 265V, 266S, 269L, 273I, 273T 273S 273M 273T 273N279T 280S 281L 284P 288L 289K 291V 294V 294D 295F 298L 310L 310P 317A 317T 327K 333A 349W 353N 355A 360E 370L 370H 383Y 395P 396H 396K 396W 396V 398F 401M 406H for representative sequence(s) shown in Table 58; D13L, 116P, L32K, N40D, 142L, A45T, A45V, Q49R, Q49R, Q49K, L54G, Y55A, Y56C, Y56L, Y56H, Y56L, T57G, 164F, 165S, 165A, R69A, M89R, V102R, K104T, Y105V, D111N, V1251, V126N, E127A, E127G, E127A, E127Y, E127I, S129W, P130A, Y131M, Y131V, Y131M, V133P, I134A, I137S, I137T, S140L, M154C, A156V, A156V, A157G, V162A, G165P, K170G, F175Y, A176G, K182t, I185K, S186G, E191L, K198M, M202L, M202R, N206A, N206L, M209C, Y213M, Y213A, K215Q, G224R, L2251, K227A, T228V, T228R, T228R, T228Q, T228V, L2291, N231Q, N231V, S232G, S232G, A239S, A239S, G240S, G240D, A241R, A241N, A241E, V246T, D250S, T251F, T251L, T251V, T251L, T251A, K256R, A257T, G258S, G258T, G260C, G260V, E2631, E263C, E263W, E263V, G264S, G264S, F267L, L2711, L271T, L271S, L271M, L271T, L271N, K277T, E278S, V279L, F282P, A286L, D287K, L289V, N292V, N292D, M293F, N296L, K308L, K308P, E316A, E316T, I326K, G332A, D348W, C352N, I354A, A359E, M369L, M369H, D382Y, R394P, K395H, K395K, K395W, K395V, H397F, Y400M, N405H for representative sequence(s) shown in Table 58; 1133R, A146R, L148T, K149V, D155N, V170N, E171A, E171G, E171A, E171Y, E171I, A173W, P174A, Y175, Y175V, Y175M, L177P, I178A, V181S, V181T, V184L, M198C, A200V, A201G, V206A, V209P, K214G, F219Y, A220G, K226t, T229K, E230G, H235L, K242t, L246R, Q250A, Q250L, I253C, K257t, K257A, K259Q, G268R, L269I, Q271A, T272V, T272R, T272R, T272Q, T272V, L273I, K275Q, K275V, S276G, S276G, A283S, A283S, G284S, G284D, A285R, A285N, A285E, V290T, D294S, T295F, T295L, T295V, T295A, H300R, A301T, A302S, A302T, A302C, A302V, E3071, E307C, E307W, E307V, G308S, G308S, F311L, L315I, L315T, L315S, L315M, L315T, L315N, K321T, E322S, V323L, L326P, A330L, D331K, L333V, H336V, H336D, M337F, H340L, Q352L, Q352P, E359A, E359T, V379K, G385A, T401W, L405N, I407A, F412E for representative sequence(s) shown in Table 58; E40D, V42L, A45T, A45V, Y49R, Y49R, Y49K, T54G, L55C, L55H, L55L, S56G, L63F, 164S, 164A, R68A, M88R, A101R, L103T, K104V, D110N, L124I, I125N, E126A, E126G, E126Y, E126I, G128W, P129A, Y130M, Y130V, Y130M, V132P, I133A, I136S, I136T, T139L, M153C, A155V, A156G, V160A, K169G, K174Y, 175G, Q181M, V184K, K185G, N190L, A197t, I201L, I201R, N205A, N205L, M208C, G212t, G212A, N214Q, G223R, V224L, V2241, K226A, A227V, A227R, A227R, A227Q, A227V, V2281, S230Q, S230V, S231G, S231G, A238S, A238S, G239S, G239D, A240R, A240N, A240E, V245T, D248S, T250F, T250L, T250V, T250A, K255R, A256T, A257S, A257T, A257C, A257V, A2621, A262C, A262W, A262V, G263S, F266L, L2701, L270T, L270S, L270, L270T, L270N, K276T, E277S, V278L, 1281P, A285L, D286K, L288V, Y291V, Y291D, M292F, Y295L, R308L, R308P, T314A, 1334K, G340A, S357W, V360N, L362A, A367E, M377L, M377H, D388Y, G402P, N403H, N403K, N403W, N403V, H405F, A408M for representative sequence(s) shown in Table 58.

Representative combinations include combinations of the single substitutions exemplified in Table 58 as in the corresponding respective sequences, including but not limited to: 12A; 12V; 139S; 139T; 204R; 361S; 396H; 417A; 129Y, 129I, 131W, 132A, 133M, 215M, 217Q, 226R, 230V, 259T; 132A, 133M, 133V, 136A, 139S, 142L, 156C, 158V, 159G, 164A, 167P, 187K, 188G, 200M; 241S, 242D, 243N, 248T, 252S, 253F, 258R, 259T; 396H, 396W; 142L, 156C; 129I, 131W; 394R, 411Y, 291V, 295F, 310L; 294V, 298L, 310L; 129A, 131W, 132A, 133M; 56G, 426G; 208L, 456A; 30N, 260V; 129I, 447P; 233Q, 291V, 466N; 355A, 479R; 273T, 439H, 468N; 117T, 446V, 12L, 310L, 446I, 464S; 139S, 204R, 241S, 396H, 417A; 253V, 437P; 58L, 269L, 298L; 139S, 204R; 67S, 231I, 273S, 447P; 129A, 427L; 139S, 204R, 417A; 91R, 184t, 427L, 464T; 253F, 427V; 333A, 429L; 139T, 204R, 241S, 396H, 417A; 67A, 217Q; 129I, 280S, 429M; 12A, 139S, 204R, 396H; 295F, 298L, 310L, 310P, 327K, 353N; 211C, 242D, 427V; 164A, 427L, 456A, 466N; 12V, 139S, 204R, 396H; 139S, 204R, 396H; 12V, 139S, 204R, 361S, 396H; 66F, 241S, 273T, 310P, 406H, 446V, 464T; 294D, 383Y, 135P, 211C, 396H, 401M; 281L, 396H, 401M; 204R, 281L, 396H, 401M; 204R, 211C, 396H, 401M; 204R, 396H, 401M; 129I, 135P, 204R, 396H; 129I, 135P, 211C; 129I, 135P, 211C, 396H; 129I, 135P, 396H; 129I, 135P, 281L, 396H; 211C, 281L, 396H, 401M; 211C, 281L, 401M; 211C, 401M; 204R, 211C, 370L, 396H; 204R, 211C, 281L, 396H, 401M; 204R, 211C, 281L, 401M; 204R, 211C, 401M; 204R, 281L, 401M; 135P, 204R, 211C, 401M; 129I, 135P, 211C, 396H, 401M; 129I, 135P, 204R, 211C; 129I, 135P, 204R, 211C, 396H; 204R, 401M; 129I, 135P, 204R, 396H, 437P; 12A, 139S, 204R, 361S, 396H; 15P, 129G, 167P, 227I, 258R, 260T, 298L, 406H, 446I; 124R, 139S, 204R, 396H, 413S; 67S, 129A, 215A, 419S; 353N, 452M; 58L, 217Q, 242D, 273N; 34K, 437P; 139S, 204R, 396H, 417A; 294V, 327K; 59G, 259T, 280S, 333A; 167P, 401M; 139T, 204R, 417A; 164A, 398F, 411W; 47V, 177Y; 172G, 360E; 158V, 317T; 57A, 273N, 360E; 113N, 193L; 215A, 416S, 446I; 132A, 178G, 310L, 370H; 172G, 327K, 464T; 294V, 295F, 327K; 44L, 446I; 204R, 396H, 417A; 12L, 187K, 317T, 441S; 158V, 441S; 215M, 327K, 406, 464T, and the like. It is readily apparent that these an other combinations can be readily made, used and screened using methods well known in the art and as disclosed herein. Based on methods well known in the art for aligning homologous sequences, as described herein, the corresponding positions for the indicated variant amino acid positions of Table 58 can be readily determined for a homologous ALD sequence.

As disclosed herein, it is well known to those skilled in the art that different organisms can exhibit codon bias that can affect expression of a gene. Therefore, any of the variants described herein can optionally be codon optimized for the desired amino acid at a particular position, as discussed above. Table 59 shows the well known genetic code, and one skilled in the art can readily incorporate alternative codons, where desired, into a variant to optimize expression and production, including increased expression and production, of a desired product in a variant host strain.

TABLE 59
Genetic code for translation from nucleotide
sequence to amino acid sequence.

Second base

| | | U | C | A | G | | |
|---|---|---|---|---|---|---|---|
| First | U | Phe | Ser | Tyr | Cys | U | Third |
| base | | Phe | Ser | Tyr | Cys | C | base |
| | | Leu | Ser | End | End | A | |
| | | Leu | Ser | End | Trp | G | |
| | C | Leu | Pro | His | Arg | U | |
| | | Leu | Pro | His | Arg | C | |
| | | Leu | Pro | Gln | Arg | A | |
| | | Leu | Pro | Gln | Arg | G | |
| | A | Ile | Thr | Asn | Ser | U | |
| | | Ile | Thr | Asn | Ser | C | |
| | | Ile | Thr | Lys | Arg | A | |
| | | Met | Thr | Lys | Arg | G | |
| | G | Val | Ala | Asp | Gly | U | |
| | | Val | Ala | Asp | Gly | C | |
| | | Val | Ala | Glu | Gly | A | |
| | | Val | Ala | Glu | Gly | G | |

In addition, as additionally described herein, one skilled in the art can readily incorporate amino acid substitutions, in particular conservative amino acid substitutions, to generate additional enzyme variants, which can be screened for desired activities and/or properties using the methods disclosed herein and/or well known in the art. Further, alignments of related sequences, such as those exemplified in Tables 53-58, can be used to guide the identification of additional amino acid positions to either vary or maintain as an invariant position using routine methods well known to those skilled in the art.

Throughout this application various publications have been referenced. The disclosures of these publications in their entireties, including GenBank and GI number publications, are hereby incorporated by reference in this application in order to more fully describe the state of the art to which this invention pertains. Although the invention has been described with reference to the examples provided above, it should be understood that various modifications can be made without departing from the spirit of the invention.

1.-56. (canceled)

57. An isolated polypeptide comprising an amino acid sequence referenced in Tables 53, 55 or 57, wherein said amino acid sequence comprises one or more variant amino acid positions set forth in Tables 54, 56 or 58 or Example XXXXIII.

58. The isolated polypeptide of claim 57, wherein said amino acid sequence other than said one or more variant amino acid positions has at least 65%, 70%, 75%, 80%, 85%, 90%, 95%, 98% or 99% sequence identity, or is identical, to an amino acids sequence referenced in Tables 53, 55 or 57, with the proviso that the amino acid sequence is not the wild type parental sequence or a naturally occurring amino acid sequence.

59. The isolated polypeptide of claim 58, wherein said amino acid sequence comprises two, three, four, five, six, seven, eight, nine, ten, eleven, twelve, thirteen, fourteen, fifteen, sixteen, seventeen, eighteen, nineteen, twenty or more variant amino acid positions set forth in Tables 54, 56 or 58 or Example XXXXIII.

60. The isolated polypeptide of claim 59, wherein said amino acid sequence further comprises a conservative amino acid substitution in from 1 to 100 amino acid positions, wherein said positions are other than the variant amino acid positions set forth in Tables 54, 56 or 58 or Example XXXXIII.

61. The isolated polypeptide of claim 57, wherein said amino acid sequence comprises no modification at from 2 to 300 amino acid positions compared to the parent sequence, wherein said positions are selected from those that are identical to between 2, 3, 4, or 5 of the amino acid sequences referenced in Tables 53, 55 or 57.

62. The isolated polypeptide of claim 57, wherein said polypeptide can convert 4-hydroxybutyrate to 4-hydroxybutyryl-CoA, 4-hydroxybutyryl-CoA to 4-hydroxybutyraldehyde, or 4-hydroxybutyraldehyde to 1,4-butanediol.

63. A host cell expressing the polypeptide of claim 57.

64. Use of the polypeptide of claim 57 as a biocatalyst.

65. A composition comprising the polypeptide of claim 57 and at least one substrate for said polypeptide.

66. The composition of claim 65, wherein said polypeptide can react with said substrate under in vitro conditions.

67. A method for producing 4-hydroxybutyrate (4-HB), gamma-butyrolactone (GBL), 4-hydroxybutyraldehyde (4-HBal), 4-hydroxybutyryl-CoA (4-HBCoA), 1,4-butanediol (BDO) and/or putrescine comprising culturing said host cell of claim 63 under conditions and for a sufficient period of time to produce 4-HB, GBL, 4-HBal, 4-HBCoA, BDO and/or putrescine.

68. The method of claim 67, wherein said non-naturally occurring microbial organism is in a substantially anaerobic culture medium.

69. A method for producing 4-HB, GBL, 4-HBal, 4-HBCoA, BDO and/or putrescine, comprising culturing a non-naturally occurring microbial organism having a pathway that produces 4-HB, GBL, 4-HBal, 4-HBCoA, BDO and/or putrescine under conditions and for a sufficient period of time to produce 4-HB, GBL, 4-HBal, 4-HBCoA, BDO and/or putrescine, wherein said microbial organism comprises the polypeptide of claim 57.

70. The method of claim 69, wherein said non-naturally occurring microbial organism is in a substantially anaerobic culture medium.
